(19) United States
(12) Patent Application Publication
(10) Pub. No.: US 2007/0072178 A1
(43) Pub. Date: Mar. 29, 2007

(54) Novel genetic markers for leukemias

(76) Inventors: Torsten Haferlach, Munich (DE); Claudia Schoch, Munich (DE); Kern Wolfgang, Starnberg (DE); Alexander Kohlmann, Neumarkt (DE); Susanne Schnittger, Munich (DE); Martin Dugas, Deggendorf (DE); Roland Eils, Schriesheim (DE); Benedikt Brors, Dossenheim (DE); Susanne Mergenthaler, Basel (CH)

Correspondence Address: QUINE INTELLECTUAL PROPERTY LAW GROUP, P.C., P O BOX 458, ALAMEDA, CA, 94501, US

(21) Appl. No.: 10/494,834

(22) PCT Filed: Nov. 4, 2002
(86) PCT No.: PCT/EP02/12303

§ 371 Date: Nov. 19, 2004

(30) Foreign Application Priority Data
Nov. 5, 2001 (EP) 01126244.1
Apr. 30, 2002 (EP) 02009758.0

(51) Int. Cl. C12Q 1/68

(52) U.S. Cl. 435/6

(57) ABSTRACT

The present invention is related to methods for detecting leukemia cells by determining the expression profile of a group of markers. In particular, the type or subtype of leukemia cells in a sample is determined. Further, uses of the group of markers is discloses and compositions comprising these markers.

The present invention is related to methods for detecting leukemia cells by determining the expression profile of a group of markers. In particular, the type or subtype of leukemia cells in a sample is determined. Further, uses of the group of markers are disclosed and compositions comprising these markers.

In the present specification, a number of documents is cited. The disclosure content of these documents including manufacturers' manuals, is herewith incorporated by reference. This holds particular true for the documents such as gene accession numbers cited in Tables 43a, b, 44 and 45 providing the complete nucleotide sequence of marker genes/cDNAs. In other terms, by reciting these documents, applicant intends to incorporate the complete nucleotide/amino acid sequence of those markers where only a partial sequence has been identified in the appended Tables. It is also intended to include the (poly)peptide sequences translated from these nucleotide sequences within the disclosure content of the present specification.

Today leukemias are classified into four different groups or types: acute myeloid (AML), acute lymphatic (ALL), chronic myeloid (CML) and chronic lymphatic leukemia (CLL). Within these groups, several subcategories can be identified further using a panel of standard techniques as described below. The incidence of leukemias is increasing with age and is 5/100.000/year in AML, 1/100.000/year in ALL, 1/100.000 in CML and 6/100.000/year in CLL. Several methods for classification have to be applied at diagnosis and before treatment starts: cytomorphology and cytochemistry, multiparameter-immunophenotyping, cytogenetics including fluorescence in situ hybridization, and molecular techniques such as polymerase chain reaction (PCR). So far only a combination of these techniques allows a precise diagnosis which is necessary to apply state of the art treatment. As the exact diagnosis is mandatory for example in CML the detection of a specific cytogenetic abnormality, the translocation (9;22) or its molecular counterpart, the BCR/ABL rearrangement is required to establish the diagnosis of CML. While all patients with CML show a BCR-ABL-rearrangement and are therefore homogenous with regard to the primary genetic abnormality, in AML and

ALL at least 10-15 different subgroups have been identified on the morphological, genetical or molecular level. Also in CLL several subgroups can be clearly separated. These different subcategories in leukemias are associated with varying clinical outcome and therefore are the basis for different treatment strategies. The importance of highly specific classification may be illustrated in detail further for the AML as a very heterogeneous group of diseases.

Data from clinical trials showed that outcome of patients with AML differs in a broad range. Several parameters influencing prognosis have been identified. These can be assigned to different categories: patients' characteristics (i.e. age, comorbidity), therapy, and biology of the AML. Therefore, a lot of effort was invested to identify biological entities and to distinguish subgroups of AML which are associated with a favorable, intermediate or unfavorable prognosis, respectively. In order to allow a comparison between different studies a classification of AML was mandatory. In 1976 the FAB classification was proposed by the French-American-British co-operative group which was based on cytomorphology and cytochemistry in order to separate AML subgroups according to the morphological appearance of blasts in the blood and bone marrow. In addition, it was recognized that genetic abnormalities occurring in the leukemic blast had a major impact on the morphological picture and even more on the prognosis. So far, the karyotype of the leukemic blasts is the most important independent prognostic factor regarding response to therapy as well as survival. For clinical purposes karyotype analysis allows to discriminate between three major prognostic groups. A favorable outcome under currently used treatment regimens with cure rates from 50% up to 858 was observed in several studies in patients with a) t (8;21) (q22; q22) occurring in AML M2, b) inv (16) (p13q22) occurring in; AML M4eo and c) t(15;17) (q22; qll-12) occurring in AML M3/H3v. In contrast, chromosome aberrations with an unfavorable clinical course are −5/del(5q), −7/del(7q), inv(3)/t(3:31 and complex aberrant karyotypes with cure rates of only 10%. The remainder of AML patients are assigned to a prognostically intermediate group. This latter group is very heterogeneous because it includes patients with a normal karyotype as well as those with rare chromosome aberrations with yet unknown prognostic impact.

The sub-classification of leukemias becomes Increasingly important to guide therapy. Thus, the development of new, specific treatment approaches requires the identification of specific subtypes that may benefit from a distinct therapeutic protocol. It has already been shown in two entities that the development of specific drugs can improve outcome of distinct subsets of leukemia. One important example is the development of a new therapeutic drug (ST1571) for the treatment of chronic myeloid leukemia (ML): this designed molecule inhibits the CML specific chimeric tyrosine kinase BCR-ABL generated from the genetic defect observed in CML, the BCR-ABL rearrangement due to the translocation between chromosomes 3 and 22 (t(9;22) (q34; q11)). First data show that therapy response is dramatically higher in patients treated with this new drug as compared to all other drugs that had been used so far. Another example is the subtype of acute myeloid leukemia AML M3 and its variant M3v both with karyotype t[15;17) (q22; q11-12). The introduction of a new drug (all-trans retinoic acid—ATRA) has improved the outcome in this subgroup of patient from about 50% to 85% long-term survivors; As it is mandatory for these patients suffering from these specific leukemia subtypes to be identified as fast as possible so that the best therapy can be applied, diagnostics today must accomplish sub-classification with maximal precision. Not only for these subtypes but also for several other leukemia subtypes different treatment approaches could improve outcome. Therefore, rapid and precise identification of distinct leukemia subtypes is the future goal for diagnostics.

So far a combination of methods is necessary to obtain the most important information in leukemia diagnostics: Analysis of the morphology and cytochemistry of bone marrow blasts and peripheral blood cells is necessary to establish the diagnosis. In some cases the addition of immunophenotyping is mandatory to separate very undifferentiated AML from acute lymphoblastic leukemia and CLL. Leukemia subtypes investigated can be diagnosed by cytomorphology alone, only if an expert reviews the smears. However, a genetic analysis based on chromosome analysis, fluorescence in situ hybridization or RT-PCR and immunophenotyping is required in order to assign all cases in to the right category. The aim of these techniques besides diagnosis is mainly to determine the prognosis of the leukemia. A major disadvantage of these methods, however, is that viable cells are necessary as the cells for genetic analysis have to divide in vitro in order to obtain metaphases for the analysis. Another problem is the long time of 72 hours from receipt of the material in the laboratory to obtain the result. Furthermore, great experience in preparation of chromosomes and even more in analyzing the karyotypes is required to obtain the correct result in at least 90% of cases. These experts in their field are necessary for all other techniques mentioned above as well. Accordingly, standard diagnosis of leukemia uses a combination of complementary methods, is expensive, time-consuming, and requires experienced experts in the field. Methods that have to be combined are cytomorphology or histomorphology, multiparameter-immunophenotyping, cytogenetics, fluorescence in situ hybridization, and molecular genetics such as polymerase chain reaction based assays.

Using these techniques in combination, hematological malignancies in a first approach are separated into chronic myeloid leukemia (CML), chronic lymphoid (CLL), acute lymphoblastic (ALL), and acute myeloid leukemia (AML). Within the latter three disease entities several prognostically relevant subtypes have been established. As a second approach this further subclassification is based mainly on genetic abnormalities of the leukemic blasts and clearly is associated with different prognoses. Therefore, this subclassification is increasingly important to guide therapy. Furthermore, the development of new, specific treatment approaches requires precise identification of leukemia subtypes.

In a first study Golub et al. (Science 1999) showed that gene expression profiles can be used for class prediction and discriminated AML from ALL samples. However, for his analysis of acute leukemias the selection of the two different subgroups was performed using exclusively morphologic-phenotypical criteria. This was only descriptive and does not provide deeper insights into the pathogenesis or the underlying biology of the leukemia. The approach reproduces only very basic knowledge of cytomorphology and intends to differentiate classes. The data is not sufficient to predict prognostically relevant cytogenetic aberrations.

Thus, the technical problem underlying the present invention was to provide means for leukemia diagnostics which overcome the disadvantages of the prior art diagnostic methods.

The solution to said technical problem is achieved by providing the embodiments characterized in the claims. Accordingly, the present invention relates to a method of determining whether a patient sample contains leukemia cells or other cells comprising the steps of a) determining the expression profile of a group of markers in a patient sample and b) concluding from the expression profile whether the patient sample contains leukemia cells or other cells characterized in that the group of markers consists of markers selected independently from the markers listed in one or more of the tables 3 to 6, tables 15 to 20, tables 29, 30, 41, or 42 and whereby the number of markers in the group is between one and the total number of markers listed in the tables 3 to 6, tables 15 to 20, and tables 29, 30, 41, or 42. In a particular embodiment thereof, the present invention pertains to a method wherein leukemia type and subtype are simultaneously determined whereby a microarray for the detection of the expression level of a marker or a group of markers is used.

It is important to note that in accordance with the invention in all pertaining embodiments any possible combination of markers, said markers being disclosed in the respective table or tables is encompassed within the scope of the invention.

As used herein, the term “expression” refers to the process by which mRNA or a polypeptide is produced based on the nucleic acid sequence of a gene. The process includes both transcription and translation, i.e. “expression” shall also include the formation of mRNA upon transcription.

In accordance with the present invention, the term “determining the expression profile” preferably refers to the determination of the level of expression, namely of said group of markers.

As used herein, the term “marker” refers to a DNA, in particular cDNA, or RNA or a fragment thereof or a protein or a fragment thereof which are in the case of RNA (or cDNA) formed upon transcription of a nucleotide sequence which is capable of expression. The nucleic acid molecule fragments refer to fragments preferably of at least 8 such as ten, twelve, fifteen or eighteen nucleotides in length representing a consecutive stretch of nucleotides of a gene, cDNA or mRNA such as of 20 or nucleotides that are, for example, further specified in the appended Tables or a complementary sequence thereto. In other terms, markers include any fragment (or complementary sequence thereto) of the sequences depicted in the appended tables as long as these fragments unambiguously identify the marker. Typical fragment lengths are provided above. The determination of the expression profile of markers may be effected at the transcriptional or translational level. In other terms, the method of the invention envisages the determination at the level of mRNA or at the protein level. Protein fragments such as peptides advantageously comprise at least 6 consecutive amino acids representative of the corresponding full length protein. 6 amino acids are generally recognized as the lowest peptidic stretch giving rise to a linear epitope recognized by an antibody, fragment or derivative thereof. Alternatively, the proteins or fragments thereof may be analysed using nucleic acid molecules specifically binding to three-dimensional structures (aptamers). In principle, the investigator may determine, in accordance with the method of the invention, whether a gene is expressed at all in a leukemic or other cell. Alternatively, an investigator may determine the difference in the expression level, for example, between a leukemic and a non-leukemic cell or between two or more different types or subtypes of leukemia. If the sample comprises only other, i.e. non-leukemia cells, then the patient's suffering from a leukaemia may safely be denied. Insofar, the above main embodiment is to be understood that if the presence of other cells is determined then this determination includes an assessment to the effect that only other cells but no leukemic cells are comprised in the sample. On the other hand, the determination of leukemic cells may include the further characterization of such cells including the differentiation status of the cells as well as the distinction from other types of cancer cells or other subtypes of leukaemia cells. Particular embodiments in this regard are further outlined herein below.

In accordance with the above, the present invention also contemplates methods where simply the assessment of leukaemia cells but not necessarily of other cells is effected. This holds true for all embodiments where the determination of other cells is mentioned. It is to be understood that with the exception of the possible determination of other cells, the steps of the various methods of the invention remain unchanged. Thus, the invention also relates to a method of determining whether a patient sample contains leukemia cells comprising the steps of a) determining the expression profile of a group of markers in a patient sample and b) concluding from expression profile whether the patient sample contains leukemia cells characterized in that the group of markers consists of markers selected independently from the markers listed in one or more of the tables 3 to 6, tables 15 to 20, tables 29, 30, 41, or 42 and whereby the number of markers in the group is between one and the total number of markers listed in the tables 3 to 6, tables 15 to 20, and tables 29, 30, 41, or 42. Thus, the invention further relates to a method of determining whether a patient sample contains leukemia cells and at the same time or subsequently determining the type and subtype of leukemia cells, if leukemia cells are present, comprising the steps of a) determining the expression profile of a group of markers in a patient sample and b) concluding from the expression profile whether the patient sample contains leukemia cells and at the same time or subsequently determining the type and subtype of leukemia cells, if leukemia cells are present, characterized in that the group of markers consists of markers selected independently from the markers listed in one or more of the tables 16 to 20 or table 29 or 30 and whereby the number of markers in the group is between one and the total number of markers listed in the tables 16 to 20 or table 29 or 30, to name two important embodiments of the invention.

Determination of the expression profile/levels may be effected by a variety of methods, depending on the nature of the marker. Thus, if the marker is mRNA, cDNA may be prepared into which a detectable label, such as a fluorescent, chemiluminescent, bioluminescent, radioactive (such as ³H or ³²P) label is incorporated. Said detectably labelled cDNA, in single-stranded form, may then be hybridised, preferably under stringent or highly stringent conditions to a panel of single-stranded oligonucleotides representing different genes and affixed to a solid support such as a chip. Upon applying appropriate washing steps, those cDNAs will be detected or quantitatively detected that have a counterpart in the oligonucleotide panel. Various advantageous embodiments of this general method are feasible. For example, the mRNA or the cDNA may be amplified wherein it is, for quantitative assessments, preferable that the number of amplified copies corresponds relative to further amplified mRNAs or cDNAs to the number of, mRNAs originally present in the cell. Also, the cDNAs may be transcribed into cRNAs wherein only in the transcription step a label is incorporated into the nucleic acid and wherein the cRNA is employed for hybridisation. Alternatively, the table may be attached subsequent to the transcription step. Similarly, proteins from a cell or tissue under investigation may be contacted with a panel of aptamers or of antibodies or fragments or derivatives thereof. The antibodies etc. may be affixed to a solid support such as a chip. Binding of proteins indicative of a leukemia or a subtype of leukaemia may be verified by binding to a detectably labelled secondary antibody or aptamer. For the labelling of antibodies, it is referred to Harlow and Lane, “Antibodies, a laboratory manual”, CSH Press, 1988, Cold Spring Harbor. As regards further test assays and formats, it is referred to further embodiments of the invention as specified herein below as well as to the appended examples. In addition, a number of applicable assay formats are available in the art that can applied to the method of the invention without further ado. Specifically, a minimum set of proteins necessary for diagnosis of all leukemia types may be selected for creation of a protein array system to make diagnosis on a protein lysate of a diagnostic bone marrow sample directly. Protein Array Systems for the detection of specific protein expression profiles already are available (for example: Bio-Plex, BIORAD, München, Germany). For this application preferably antibodies against the proteins have to be produced and immobilized on a platform e.g. glasslides or microtiterplates. The immobilized antibodies can be labeled with a reactant specific for the certain target proteins as discussed above. The reactants can include enzyme substrates, DNA, receptors, antigens or antibodies to create for example a capture sandwich immunoassay.

The level of the expression of the “marker” is indicative of a leukemic condition, of a cell or an organism. The level of expression of a marker or group of markers is measured and is compared with the level of expression of the same marker or the same group of markers from other cells or samples. The comparison may be effected in an actual experiment or in silico. When the expression level also referred to as expression pattern or expression signature (expression profile) is measurably different, there is according to the invention a meaningful difference in the level of expression. Preferably the difference at least is 5%, 10% or 20%, more preferred at least 50% or may even be as high as 75% or 100%. More preferred the difference in the level of expression is at least 200%, i.e. two fold, at least 500%, i.e. five fold, or at least 1000%, i.e. 10 fold.

The present invention allows to diagnose a wide variety and at least 14 different clinically relevant leukemia subtypes. Therefore, the invention of a combination of marker genes and their specific expression level it is possible to substitute all other mandatory diagnostic approaches including the approach of Golub and colleagues (cytomorphology or histomorphology, multiparameter-immunophenotyping, cytogenetics, fluorescence in situ hybridization, and molecular genetics) in one single step with a specificity and sensitivity that had never been achieved in all other techniques used so far.

In more detail, based on biomathematical analysis of gene expression profiles a new method could be provided which forms the basis for designing and developing a novel diagnostic approach preferably based on microarray technology. Further, subsets of markers, preferably genes could be introduced which allow the determination of leukemia type and subtype. The method according to the invention abolishes today's standard procedures in diagnosis of leukemia. These standard diagnostic procedures require more and more centralized core facilities with both personal experts in the fields of cytomorphology, cytogenetics and molecular genetics and expensive lab equipment, which causes increasing costs for adequate diagnosis. The present invention provides novel cost-effective methods and diagnostic tools, which are less time consuming, easy to operate but nevertheless as accurate and safe as all standard methods combined today. The genes or sets of genes allows to assign clinical samples either as healthy or malignant simply based on their gene expression profiles. The genes, representative fragments thereof or transcription or translation products thereof form the basis for the methods of the invention or diagnostic tools, corresponding thereto. Furthermore, these genes etc. allow to predict the diagnoses based on the genetic abnormality of the expression pattern and to discriminate between different prognostic relevant entities. When comparing two groups of microarray experiments, Golub's method (Science 286 (1999), 531-537) sorts the genes with respect to the signal-to-noise ratio of gene x: Sₓ=(μ₁-μ₂)/(σ₁+σ₂), where μₖ and σₖ denote the mean expression and standard deviation of gene x in group k.

According to a specified number of “informative” genes the 20 best discriminating genes are selected. For each informative gene a decision limit is calculated as bₓ=(μ₁+/μ₂)/2. To classify a new sample of an independent test set, the gene expression levels of informative genes are taken and for each gene x and sample y a so-called vote is calculated as Vₓ=Sₓ(gₓ^{y}−bₓ), where gₓ^{y} denotes expression level of gene x in sample y. The votes of all informative genes are summed up (“weighted voting”) and depending upon the sign of this sum the new sample is classified as group 1 or group. 2. The confidence in the prediction is calculated as |ΣVₓ/Σ|Vₓ||.

To assess the significance of each gene, a permutation test is performed, which determines signal-to-noise ratios when class labels are permuted randomly.

To assess the robustness of the classifier, a leave-one-out crossvalidation is performed. Accuracy is the rate of correctly classified test samples.

The decision limit proposed by Golub does not provide optimal classification accuracy in all situations. When the standard deviation of expression levels within the two groups are very different, the decision limit is biased towards the group with the higher standard deviation.

A decision limit for a particular gene can be considered optimal, if it achieves maximum classification accuracy for a given dataset. By determining systematically classification accuracies for a set of possible decision limits, an optimal decision limit can be calculated. The underlying statistics as described in Example 3 select an optimal decision limit from the following set of decision limits Lₓ:
Lₓ={(gₓ^{y}+gₓ^{y-1})/2|1<y<=n}
where gₓ^{y} denotes expression level of gene x in sample y, n denotes the total number of samples in the training set.

Golubs method selects an arbitrary number of “informative” genes to discriminate between two classes of samples according to their signal-to-noise ratio, typically in the range of 10 to 50 genes.

Choosing too many genes like in Golub's method carries the risk of overfitting, which causes poor generalization features of the model.

Therefore the present invention applies an heuristic approach to select a minimal set of discriminative genes, which provides maximum classification accuracy in leave-one-out-crossvalidation. I.e. for a given set of genes weighted voting as described by Golub is applied and the classification accuracy is calculated by crossvalidation used in accordance with the present invention and representing a further embodiment in accordance with this invention.

The method for achieving this used in accordance with the present invention and representing a further embodiment in accordance with this invention consists of the following steps:
(a) calculating of the top 20 discriminating genes according to the signal-to-noise ratio (top 20 SNR's);
(b) calculating classification accuracy and confidence based on optimal decision limits for each of the top 20 genes;
(c) selecting the gene which provides best classification accuracy and confidence out of step 2; and
(d) testing for each of the remaining 19 genes, whether adding this gene to the model improves accuracy and confidence.

If the gene improves accuracy and confidence, it is added to the weighted voting model, otherwise it is discarded.

Preferably, the decision limit is set according to the formula recited above.

In a pilot study consisting of 103 Affymetrix Genechip microarrays with 12625 genes each as shown in the appended examples we compared the results achieved with Golub's method and with our extended method.

Table A presents an analysis of 18 samples class A versus 85 samples class non-A. Based on 20 informative genes Golub's method results in a crossvalidation accuracy of 0,87 (confidence 0,77); achieves with three genes out of the top 20 set a crossvalidation accuracy of 0,96 (confidence 0,88).

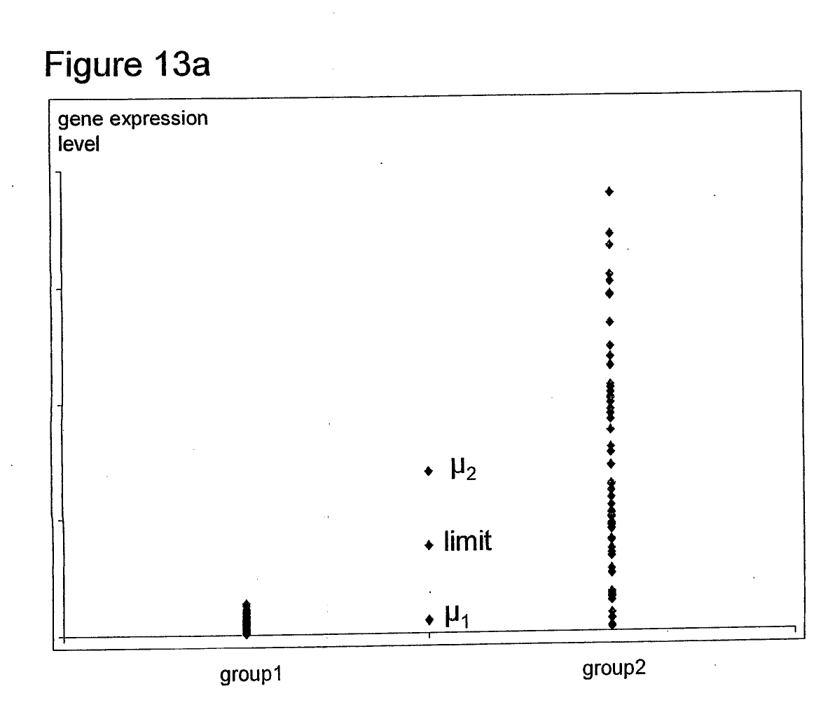

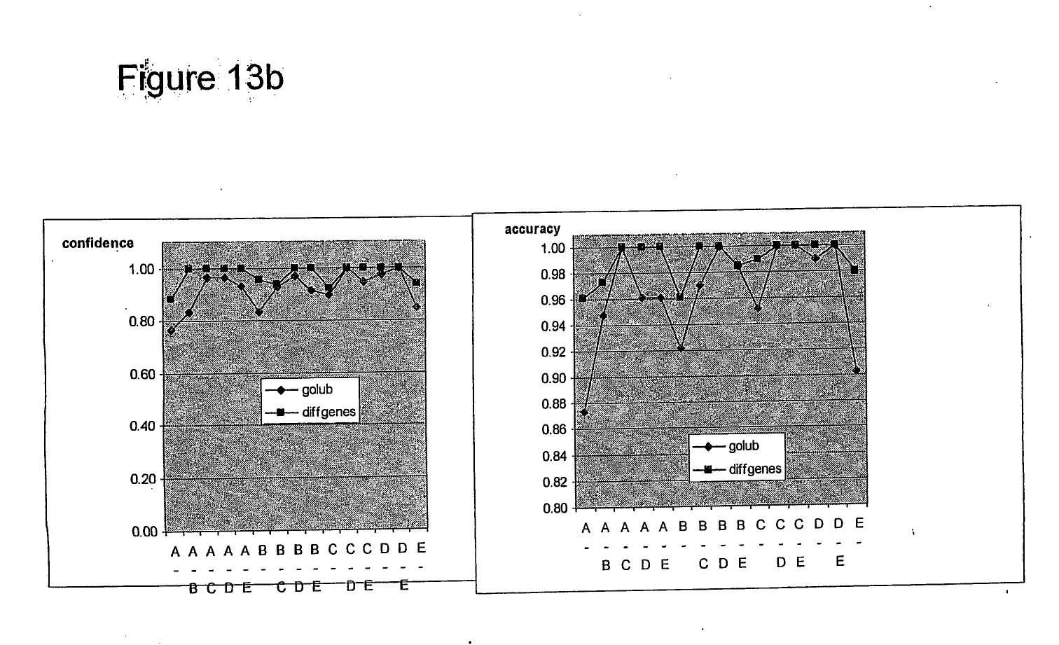

The same analysis was performed for one versus all (OVA) and all pairs (AP) comparisons in this dataset consisting of 5 different classes. FIG. 13b presents accuracy and confidence obtained by both methods: the method of the invention outperforms Golub's method clearly both in terms of accuracy and confidence of classifications.

The development of a leukemia diagnostic tool, preferably microarray based, allows for all patients which are preferably humans and specimens a reproducible, highly specific and rapid method to obtain important information for treatment strategies in leukemia. This technique can be established in every laboratory using basic methods of molecular biology, and preferably makes use of hybridization and amplification such as PCR or LCR based techniques and does not require hematologists or cytogeneticists with several years of experience in leukemia diagnostics. Material for the analysis can be sent over large distances as it is not necessary that cells arrive viable in the laboratory. Therefore, a centralization of leukemia diagnostics with very high quality is possible.

Moreover, the accumulation of an immense knowledge about gene expression profiles in leukemia types and subtypes, which are not characterized by specific genetic abnormalities, leads to a more precise classification compared to all other methods used so far. In addition, the data compiled in accordance with the invention are helpful for the understanding of the pathogenesis of leukemia and will allow to identify genes which are specifically dysregulated. They may be considered as potential targets for therapeutic interventions specifically designed for the different leukemia subtypes.

Preferably the method according to the invention is characterized in that the group of markers consists of between two, such as three, four, five, six, seven, eight, nine or ten and the total number of markers listed in one or more of the tables 3 to 6, tables 15 to 20, and tables 29, 30, 41, or 42. Most preferred, the group consists of all markers listed in one or more tables, whereby the tables are selected from the tables 3 to 6, tables 15 to 20, and tables 29, 30, 41, or 42. The invention also contemplates that all markers in all tables are analysed. This holds true for the presently discussed as well as for embodiments discussed further below.

Another embodiment of the invention relates to a method of determining whether a patient sample contains leukemia cells or other cells and at the same time or subsequently determining the type and subtype of leukemia cells, if leukemia cells are present, comprising the steps of determining the expression profile, preferably the level of expression of a group of markers in a patient sample and concluding from the (altered) expression profile i.e. the difference in the level of expression, whether the patient sample contains leukemia cells or other cells and at the same time determining the type and subtype of leukemia cells, if leukemia cells are present, characterized in that the group of markers consists of markers selected independently from the markers listed in one or more of the tables 16 to 20 or table 29 or 30 and whereby the number of markers in the group is between one, preferably two such as three, four, five, six, seven, eight, nine or ten and the total number of markers listed in one or more of the tables 16 to 20 or table 29 or 30. It is preferred that the group of markers consists of all markers listed in one or more tables, whereby the tables are selected from the tables 16 to 20 or table 29 or 30. In a preferred embodiment it is differentiated between four types of leukemia cells and the other cells in the patient sample. The other cells are preferably normal cells.

The “other cells” may be, for example, cells affected by a disease which is not a leukaemia. It is preferred, in accordance with the present invention that said other cells are normal cells, i.e. cells not affected by any disease.

This embodiment of the present invention allows for the differentiation between four different types of leukemias, i.e. AML, CLL, CML and ALL. As has been surprisingly demonstrated in accordance with the present invention, the qualitative and/or quantitative determination of an expression profile of a number of genes [symbol] the unambiguous classing with any of the above and currently established [symbol] of leukemias. In principle and more preferred, the relation of the gene [symbol] profile to the leukaemia type may take place at the same time at which [symbol] determination of the leukaemia cells in the sample takes place. Alternatively, the classification may be effected at a later time point. It was surprising that the [symbol] nction between the large number of leukemia types and subtypes, including [symbol] cytogenetically and immunophenotypically defined, as well as types characterized by complex chromosomal aberations, could be accomplished preferably by the use of a microarray for the detection of the expression level of a marker or a group of markers with such ease and accuracy. In particular, certain preferred subsets of genes are provided which can either be used to determine the leukemia type and subtype, or only determine the subtypes of a certain leukemia type or differentiates certain types or subtypes, respectively, from one another.

In another embodiment a method is disclosed which allows differentiating between two types of leukemia cells or one type of leukemia cells and normal cells or non-leukemia cells in a patient sample comprising the steps of determining the expression profile preferably the level of expression, of a group of markers in the patient sample and concluding from the (altered) expression profile, i.e. the difference in the level of expression, which type of leukemia cells the patient sample contains or whether it contains (only) normal cells or non-leukemia cells characterized in that the group of markers consists of markers selected independently from the markers listed in one or more of the tables 3 to 6 or tables 7 to 12 and whereby the number of markers in the group is between one, preferably two such as three, four, five, six, seven, eight, nine or ten and the total number of markers listed in one or more of the tables 3 to 6 or tables 7 to 12. In a preferred embodiment the group of markers consists of all markers listed in one or more of the tables 3 to 6 or tables 7 to 12.

In another embodiment of the invention a method is disclosed allowing the differentiation between the subtypes of AML cells or between the subtypes of AML cells and normal cells in a patient sample comprising the steps of determining the expression profile, preferably the level of expression of a group of markers in the patient sample and concluding from the (altered) expression profile, i.e. the difference in the level of expression, which subtypes of AML cells the patient sample contains or whether it contains normal cells characterized in that the group of markers consists of markers selected independently from the markers listed in one or more of the tables 1, 2, 13, 14, 17, 25, 27, 35 and 36 and whereby the number of markers in the group is between one, preferably two such as three, four, five, six, seven, eight, nine or ten and the total number of markers listed in one or more of the tables 1, 2, 13, 14, 17, 25, 27, 35 and 36. In a preferred embodiment the group of markers consists of all markers listed in one or more of the tables 1, 2, 13, 14, 17, 25, 27, 35 and 36. It is preferred that three, four or more subtypes of AML cells are determined.

In another embodiment of the invention a method is disclosed allowing the differentiation between and thus the determination of the subtypes of ALL cells in a patient sample comprising the steps of (a) determining the level of expression of a group of markers in the patient sample and (b) concluding from the differences in the level of expression which subtypes of ALL cells the patient sample contains whereby the group of markers consists of markers selected independently from the markers listed in one or more of the tables 18, 32 or 33 and whereby the number of markers in the group is between one, preferably two such as three, four, five, six, seven, eight, nine or ten and the total number of markers listed in one or more of the tables 18, 32 or 33. It is preferred that the group of markers consists of all markers listed in one or more of the tables 18, 32 or 33.

In another embodiment of the invention a method is disclosed allowing the differentiation between and thus the determination of the subtypes of CLL cells in a patient sample comprising the steps of determining the level of expression of a group of markers in the patient sample and concluding from the differences in the level of expression which subtypes of CLL cells the patient sample contains whereby the group of markers consists of markers selected independently from the markers listed in one or more of the tables 38 or 39 and whereby the number of markers in the group is between one, preferably two such as three, four, five, six, seven, eight, nine or ten and the total number of markers listed in one or more of the tables 38 or 39. It is preferred that the group of markers consists of all markers listed in one or more of the tables 38 or 39.

In another embodiment of the invention, a method is disclosed of assessing the efficacy of a test compound for inhibiting leukemia, the method comprising comparing the expression profile of a group of markers in a first sample obtained from the patient and maintained in the presence of the test compound and the expression profile of a group of markers in a second sample obtained from the patient and maintained in the absence of the test compound, wherein a significantly altered expression profile of the group of markers in the first sample, relative to the second sample, is an indication that the test compound is efficacious for inhibiting leukemia in the patient characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two such as 3, 4, 5, 6, 7, 8, 9 or 10 and the total number of markers listed in the tables 1 to 20, tables 25 or 27 or tables 29, 30, 32, 33, 35, 36, 38, 39, 41, 42.

In accordance with this embodiment of the present invention, it is again preferred that in the comparison of expression profiles expression levels and differences in expression levels are determined and compared. It is further preferred that the alteration determined in accordance with the method of the invention in the expression profile or expression level must be in the direction of the expression profile of normal cells or at least diseased but non-leukemic cells. More preferably the alteration should be in the direction of normal blood cells, more preferably cells of the certain type. Accordingly, it is also preferred that the comparison includes an internal standard of expression levels of analysed markers wherein the internal standard represents the expression profile of non-leukemic and preferably normal cells. The comparison may be effected by relying on actual experimental data or on in silico obtained reference data.

In another embodiment of the invention a method is disclosed of assessing the efficacy of a therapy for inhibiting leukemia in a patient, the method comprising comparing the expression profile, preferably the level of expression of a group of markers in the first sample obtained from the patient prior to providing at least a portion of the therapy to the patient and the expression profile, preferably the level of expression of a group of markers in a second sample obtained from the patient following provision of the portion of the therapy, wherein a significantly (altered) expression profile, i.e. a significantly (altered) difference in the level of expression of the group of markers in the second sample, relative to the first sample, is an indication that the therapy is efficacious for inhibiting leukemia in the patient characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two such as 3, 4, 5, 6, 7, 8, 9 or 10 and the total number of markers listed in the tables 1 to 20, tables 25 or 27 or tables 29, 30, 32, 33, 35, 36, 38, 39, 41, or 42.

As with the previous embodiment, the alteration determined in accordance with the method of the invention in the expression profile or expression level must be in the direction of the expression profile or normal cells or at least diseased but non-leukemic cells. Accordingly, it is also preferred in accordance with this embodiment that the comparison includes an internal standard of expression levels of analysed markers wherein the internal standard represents the expression profile of non-leukemic and preferably normal cells. The comparison may—again—be effected by relying on actual experimental data or on in silico obtained reference data.

Within the therapy of the patient, compounds may be administered that have at least passed phase II and preferably are within phase III of clinical trials. Advantageously, in one embodiment, a therapeutical composition or medicinal product is administered that comprises one pharmaceutically active compound. In alternative embodiments, pharmaceutical compositions or medicinal products are administered that comprise more than one pharmaceutically active compound. If the composition or product comprises more than at least one pharmaceutically active compound then one of the compounds may aim at the direct reduction of tumor load wherein at least one further compound may fulfil an accessory function such as the general stimulation of the immune system. Compounds of the latter class are also well known in the art and comprise plant derived products as well as immunostimulatory molecules selected from the group of interleukins, interferons and others.

Additionally, the invention contemplates a method of refining a compound identified by the method as described herein above, said method comprising optionally the steps of said methods and:
(1) identification of the binding sites of the compound and the target molecule by site-directed mutagenesis or chimeric protein studies;
(2) molecular modeling of both the binding site of the compound and the binding site of the target molecule; and
(3) modification of the compound to improve its binding specificity for the target.

The target may in accordance with the above be DNA, mRNA or protein. All techniques employed in the various steps of the method of the invention are conventional or can be derived by the person skilled in the art from conventional techniques without further ado. Thus, biological assays based on the herein identified nature of the proteins/(poly)peptides may be employed to assess the specificity or potency of the drugs wherein the increase of one or more activities of the proteins/(poly)peptides may be used to monitor said specificity or potency. Steps (1) and (2) can be carried out according to conventional protocols. A protocol for site directed mutagenesis is described in Ling M M, Robinson B H. (1997) Anal. Biochem. 254: 157-178. The use of homology modeling in conjunction with site-directed mutagenesis for analysis of structure-function relationships is reviewed in Szklarz and Halpert (1997) Life Sci. 61:2507-2520. Chimeric proteins are generated by ligation of the corresponding DNA fragments via a unique restriction site using the conventional cloning techniques described in Sambrook (1989), loc. cit. A fusion of two DNA fragments that results in a chimeric DNA fragment encoding a chimeric protein can also be generated using the gateway-system (Life technologies), a system that is based on DNA fusion by recombination. A prominent example of molecular modeling is the structure-based design of compounds binding to HIV reverse transcriptase that is reviewed in Mao, Sudbeck, Venkatachalam and Uckun (2000). Biochem. Pharmacol. 60: 1251-1265.

For example, identification of the binding site of said drug by site-directed mutagenesis and chimerical protein studies can be achieved by modifications in the (poly)peptide primary sequence that affect the drug affinity; this usually allows to precisely map the binding pocket for the drug.

As regards step (2), the following protocols may be envisaged: Once the effector site for drugs has been mapped, the precise residues interacting with different parts of the drug can be identified by combination of the information obtained from mutagenesis studies (step (1)) and computer simulations of the structure of the binding site provided that the precise three-dimensional structure of the drug is known (if not, it can be predicted by computational simulation). If said drug is itself a peptide, it can be also mutated to determine which residues interact with other residues in the (poly)peptide of interest.

Finally, in step (3) the drug can be modified to improve its binding affinity or ist potency and specificity. If, for instance, there are electrostatic interactions between a particular residue of the (poly)peptide of interest and some region of the drug molecule, the overall charge in that region can be modified to increase that particular interaction.

Identification of binding sites may be assisted by computer programs. Thus, appropriate computer programs can be used for the identification of interactive sites of a putative inhibitor and the (poly)peptide by computer assisted searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. Modifications of the drug can be produced, for example, by peptidomimetics and other inhibitors can also be identified by the synthesis of peptidomimetic combinatorial libraries through successive chemical modification and testing the resulting compounds. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of activators of the expression of the (poly)peptide of the invention can be used for the design of peptidomimetic activators, e.g., in combination with the (poly)peptide of the invention (Rose, Biochemistry 35 (1996), 12933-12944; Rutenber, Bioorg. Med. Chem. 4 (1996), 1545-1558).

In accordance with the above, in a preferred embodiment of the method of the invention said at least one compound is further refined by peptidomimetics.

The invention furthermore relates to a method of modifying a compound identified or refined by the method as described herein above as a lead compound to achieve (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophylic moieties, or (viii) introduction/exchange of substituents on aromates or side chains, change of substituent pattern, or (ix) modification by introduction of isosteric or bioisosteric moieties, or

(x) synthesis of homologous compounds, or (xi) introduction of branched side chains, or (xii) conversion of alkyl substituents to cyclic analogues, or (xiii) derivatisation of hydroxyl group to ketales, acetales, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiozolidinesor combinations thereof; said method optionally further comprising the steps of the above described methods.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The invention moreover relates to a method of producing a pharmaceutical composition comprising optionally the steps of the aforementioned methods and further the step of formulating the at least one compound identified, refined or modified by the method of any of the preceding embodiments with a pharmaceutically active carrier or diluent.

The pharmaceutical composition produced in accordance with the present invention may further comprise a pharmaceutically acceptable carrier and/or diluent and/or excipient. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the exact intended use of the pharmaceutical composition.

The above methods referring to downstream developments also apply to therapeutically effective compounds referred to in additional embodiments herein below.

In another embodiment of the invention a method is disclosed of selecting a composition for inhibiting leukemia in a patient, the method comprising separately maintaining aliquots of cells of a patient sample in the presence of a plurality of test compositions, comparing the expression profile, preferably the level of expression of a group of markers in each of the aliquots, and selecting one of the test compositions which induces an altered expression profile of the group of markers in the aliquot containing that test composition, relative to other test compositions characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two such as 3, 4, 5, 6, 7, 8, 9 or 10 and the total number of markers listed in the tables 1 to 20, tables 25 or 27 or tables 29, 30, 32, 33, 35, 36, 38, 39, 41, 42.

Again, as with the previously recited embodiments, the alteration determined in accordance with the method of the invention in the expression profile or expression level must be in the direction of the expression profile of normal cells or at least diseased but non-leukemic cells. Accordingly, it is also preferred in accordance with this embodiment that the comparison includes an internal standard of expression levels of analysed markers wherein the internal standard represents the expression profile of non-leukemic and preferably normal cells. The comparison may—again—be effected by relying on actual experimental data or on in silico obtained reference data.

The expression “in the direction of the expression profile of normal cells” as used herein preferably relates to cells that comprise blood cells, more preferably a single type of blood cells. Most preferably, the single type of cells corresponds to the type of the leukemic cell. For example, an AML type of leukemic cell would preferably be compared to a healthy myeloic blast cell whereas a ALL type of leukemic cell would preferably be compared to a healthy lymphatic blast cell. Myeloic blast cells and lymphatic blast cells may be isolated from healthy bone marrow using well known methods, such as cell sorting based on flow cytometry using established cell surface markers.

In this method of the invention, it is preferred that the test composition comprises only one putatively active test compound. Insofar, the correlation with the activity of the test compound and the readout is particularly convenient. If the test composition comprises more than one putatively pharmaceutically active compounds, it may be considered to separately test each compound in a composition that has tested positive in a first round of the assay. Consequently, in a second round, i.e. in a repetition of steps (a) and (b), the various compositions tested positive, if any, in the first round, may be subdivided into single compounds and these single compounds tested again for their efficacy. The goal of such an approach, of course, is to obtain a composition comprising a single active compound only.

In another embodiment a method of determining new subtypes of leukemia cells is disclosed, the method comprising determining. the expression profile, preferably the level of expression of a group of markers of leukemia cells of unknown subtype, comparing the expression profile to the level of expression, ie. the expression profile, of a group of markers of leukemia cells of known subtype, thereby concluding that a new subtype is determined when the expression profile, preferably the level of expression is different to all known subtypes characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two and the total number of markers listed in the tables 1 to 20, tables 25 or 27 or tables 29, 30, 32, 33, 35, 36, 38, 39, 41, 42.

The term “subtype of leukemia cells” in accordance with the present invention may be better understood in accordance with the following Leukemias are subdivided according to their natural clinical course into acute and chronic leukemias. Based on the cell line they are derived from they are further subdivided into myeloid and lymphatic leukemias. This results in four leukemia types, i.e. acute myeloid leukemia (AML), acute lymphoblastic leukemia (ALL), chronic myeloid leukemia (CML), and chronic lymphatic leukemia (CLL). Based on genetic, phenotypic, and biological characteristic, which are assessed by cytomorphology, cytochemistry, cytogenetics, immunophenotyping, and molecular genetics, AML, ALL, and CLL are further subdivided into subtypes. These subtypes are associated with highly differing prognoses. Treatment approaches specific for these subtypes are applied and are being further optimized. Thus, an exact diagnosis based on a reliable and reproducible method is essential for the selection of the appropriate subtype-specific treatment.

The new subtypes identified in accordance with the invention may then be subjected in the same or in further patients to the other methods/embodiments of the invention.

In another embodiment a method is disclosed for guiding the therapy of leukemia in a patient depending on the leukemia subtype and/or the risk of relapse of disease, the method comprising determining the expression profile, preferably the level of expression of a group of markers in the patient sample, and deciding about the therapy strategy depending on the leukemia subtype or the risk of relapse of disease characterized in that the group of markers consists of markers selected independently from the markers listed in one or more of the 1 to 20, tables 25 or 27 or tables 29, 30, 32, 33, 35, 36, 38, 39, 41, 42 and whereby the number of markers in the group is between one, preferably two such as 3, 4, 5, 6, 7, 8, 9 or 10 and the total number of markers listed in the tables 1 to 20, tables 25 or 27 or tables 29, 30, 32, 33, 35, 36, 38, 39, 41, 42.

This embodiment is particularly important for the quick and reliable recovery of the patient from the leukemia that effects him or her. As has been stated above, the early and reliable diagnosis of the leukaemia type or subtype is particularly important for the instigation of a useful and straightforward treatment regimen. An incorrect diagnosis may result in the application of a wrong treatment regimen which, in turn, may lead to significant health risks including premature death of the patient. In accordance with the present invention, a reliable means has been provided that, based on the inventive selection of markers provided, will overcome the prior art problems of an insecure or an inappropriate time frame demanding diagnosis. In particular, the present method of the invention provides in step (a) an unambiguous and safe basis for the decision step (b). Again, the patient may safely rely on the conclusion drawn in step (b) due to the strong inherent correlation that has been achieved between the selection of markers and the leukemia subtype. The relation of tables to leukemia subtypes has also been demonstrated elsewhere in this specification.

In another embodiment of the invention, a method for monitoring the progression of leukemia in a patient is disclosed, the method comprising determining the expression profile, preferably the level of expression of a group of markers in a patient sample at a first point in time, and repeating this step at a subsequent point in time; and comparing the expression profile, preferably the level of expression detected in the previous steps and therefrom monitoring the progression of leukemia in the patient, characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two such as 3, 4, 5, 6, 7, 8, 9 or 10 and the total number of markers listed in the tables 1 to 20, tables 25 or 27 or tables 29, 30, 32, 33, 35, 36, 38, 39, 41, 42. In a preferred embodiment, the patient has undergone chemotherapy between the first point in time and the subsequent point in time (including repetitions of step (b).

In this embodiment of the present invention, the skilled artisan may repeat step (b) one or more times in order to collect additional data from different (more) time points. The additional data obtained by such further measurements may provide an overall better overview on the progress of the disease.

In accordance with this embodiment of the disease, the term “progression of leukemia” includes the interpretation of “regression of leukemia”, i.e. includes the interpretation of a negative progression. This is of course in line with the aim of the therapy and the desire of the patient.

In the methods according to the invention it is preferred that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two and the total number of markers listed in the at least one of tables 1 to 20, tables 25 or 27 or tables 29, 30, 32, 33, 35, 36, 38, 39, 41, 42. In a preferred embodiment, the number of markers in the group is between five, more preferably between 7, 10 or 15 and the total number of markers listed in the tables 1 to 20, tables 25 or 27 or tables 29, 30, 32, 33, 35, 36, 38, 39, 41, 42. It is feasible that the group of markers not only consists of those markers but also comprises them as the data will then be still statistically significant, i.e. the preferred groups may additionally contain 10, 50 or 100 other markers and comprise the other markers according to the invention and mentioned above. It is, however, also feasible for the expert skilled in the art that only a single suitable marker is determined with the methods according to the invention.

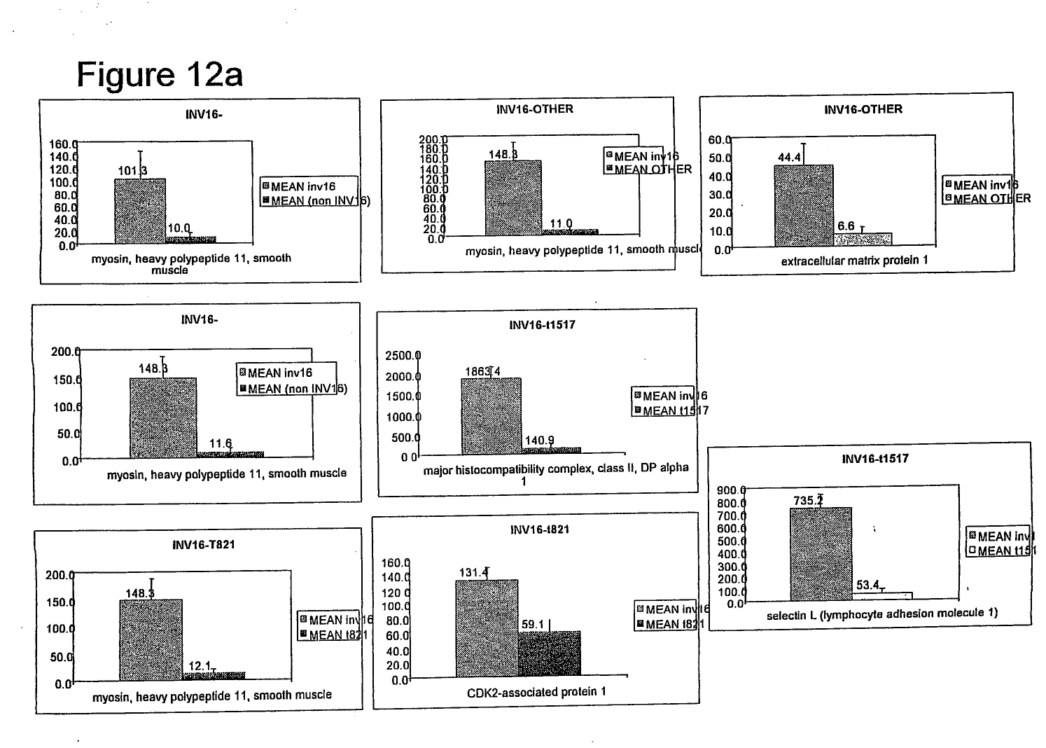

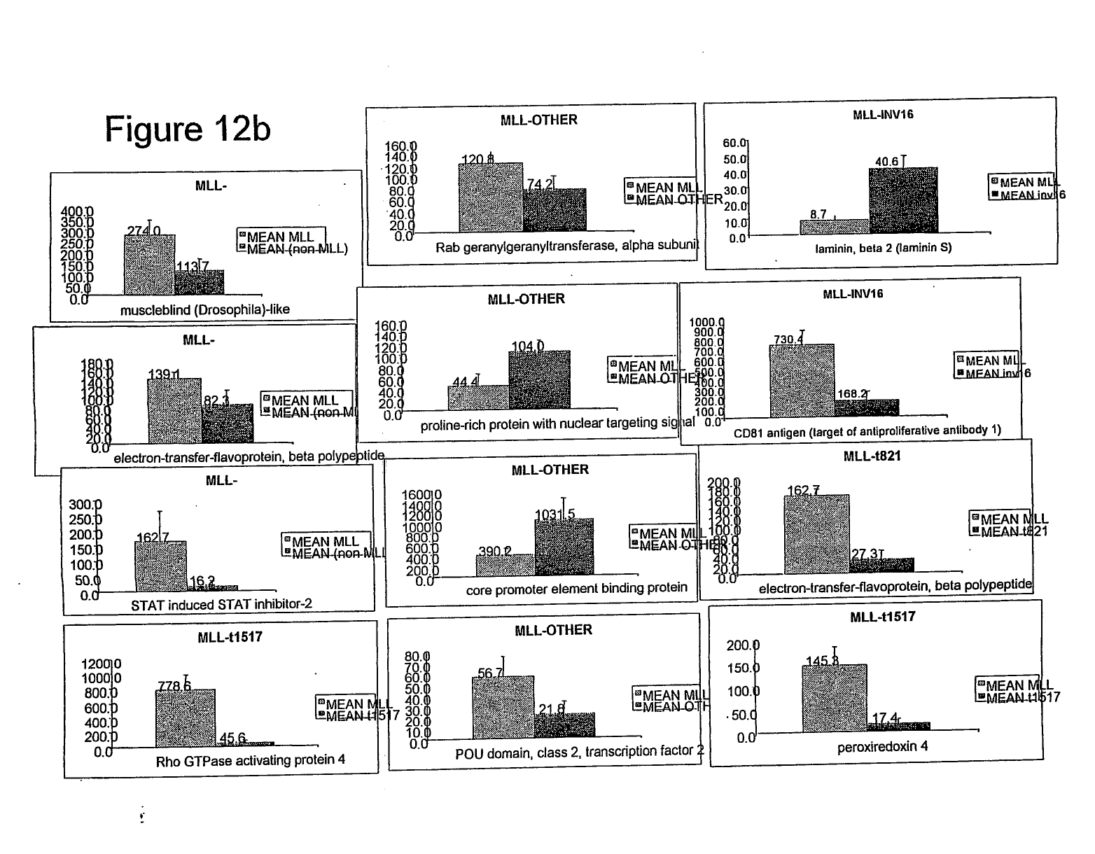

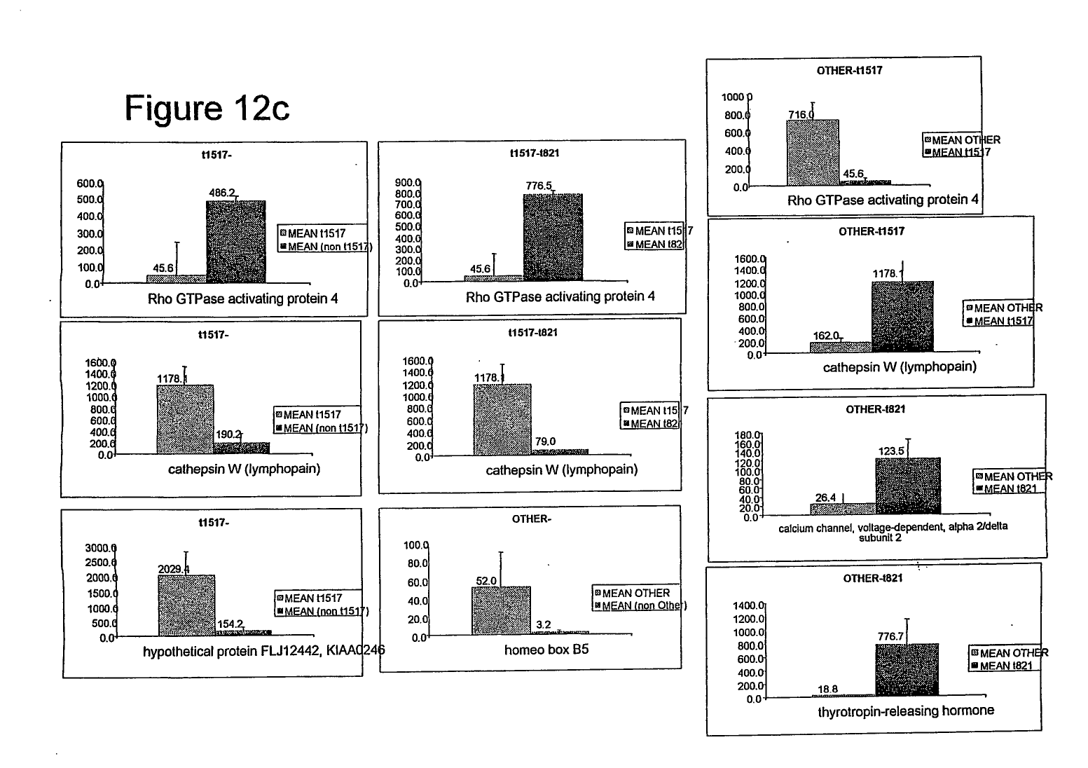

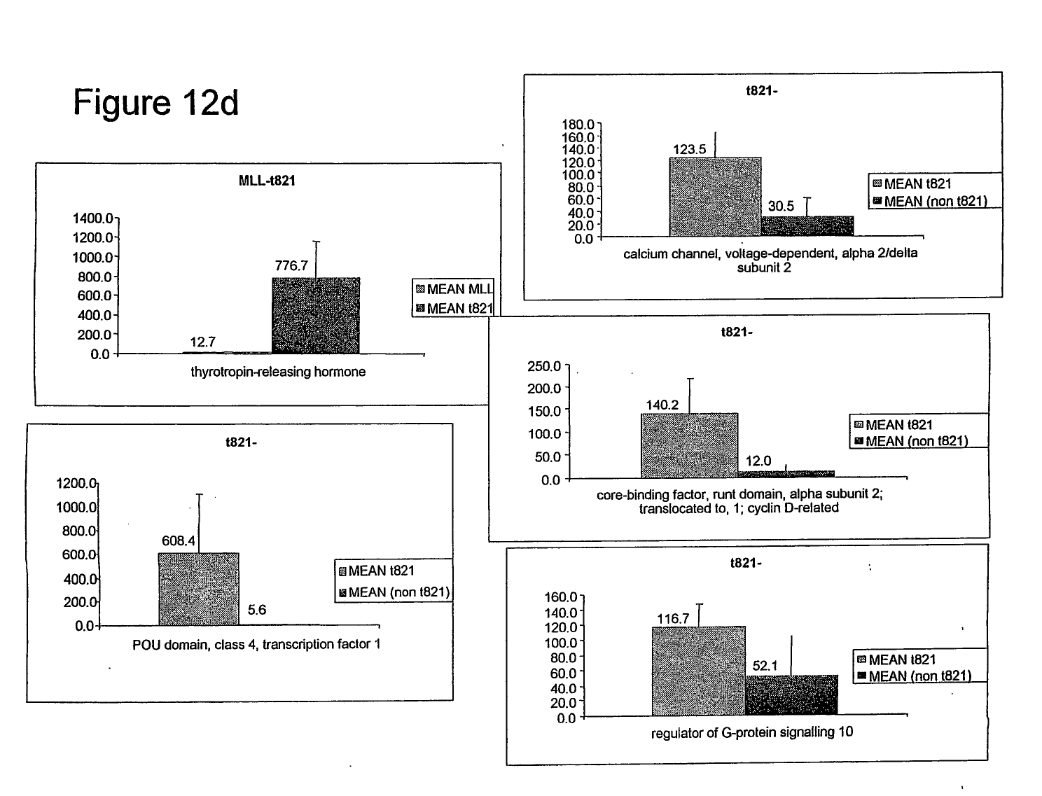

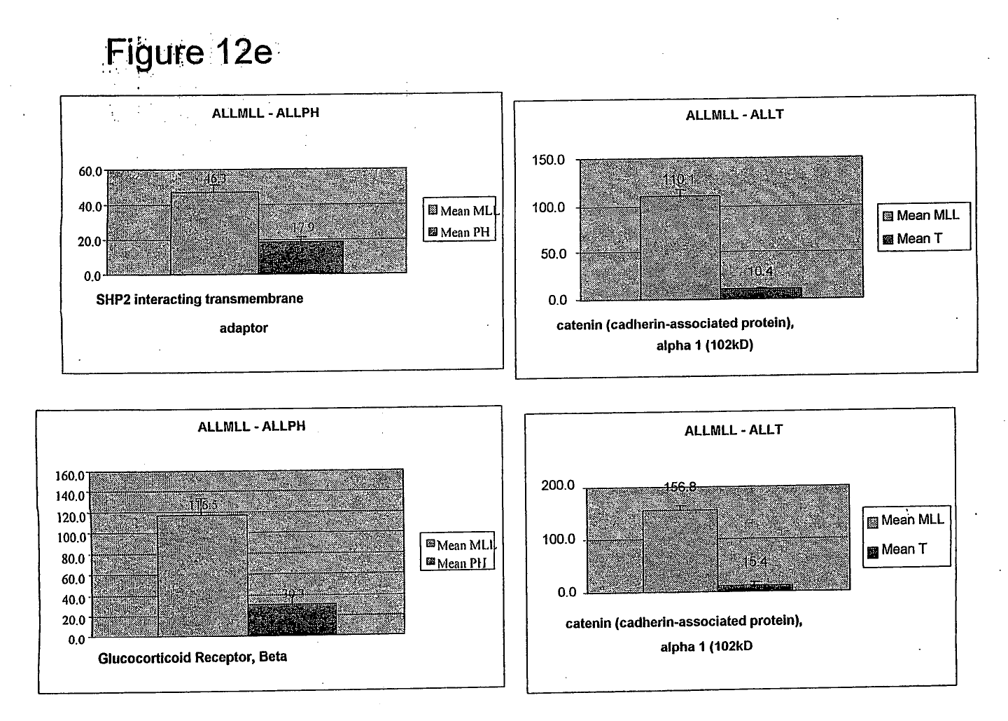

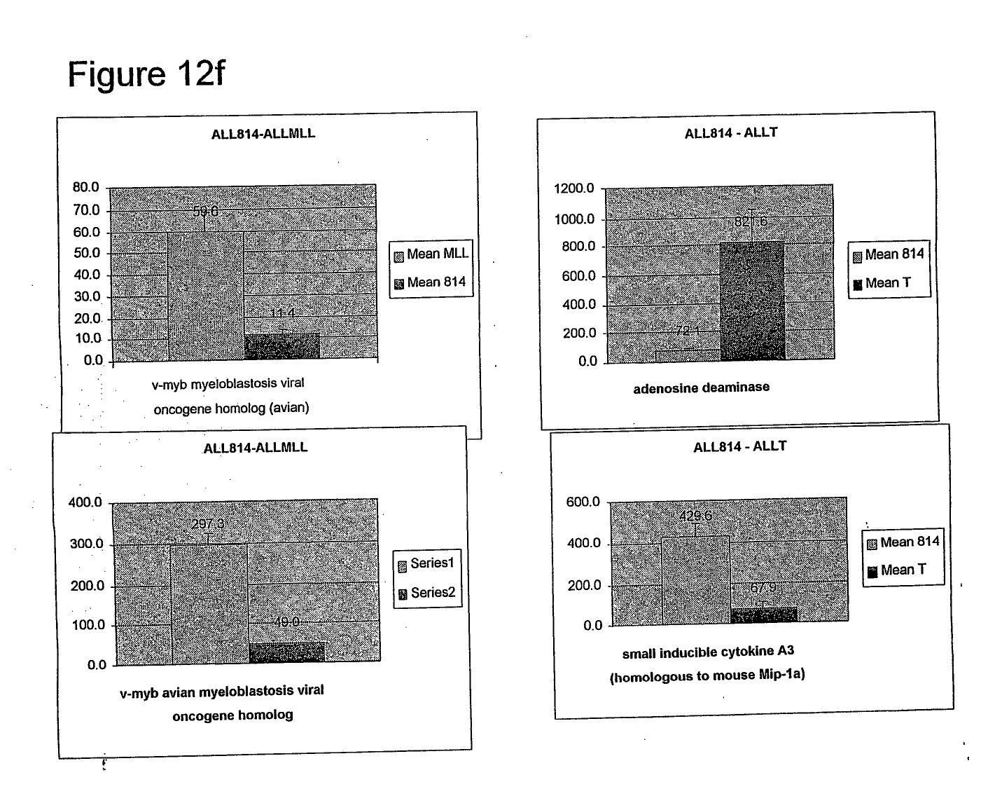

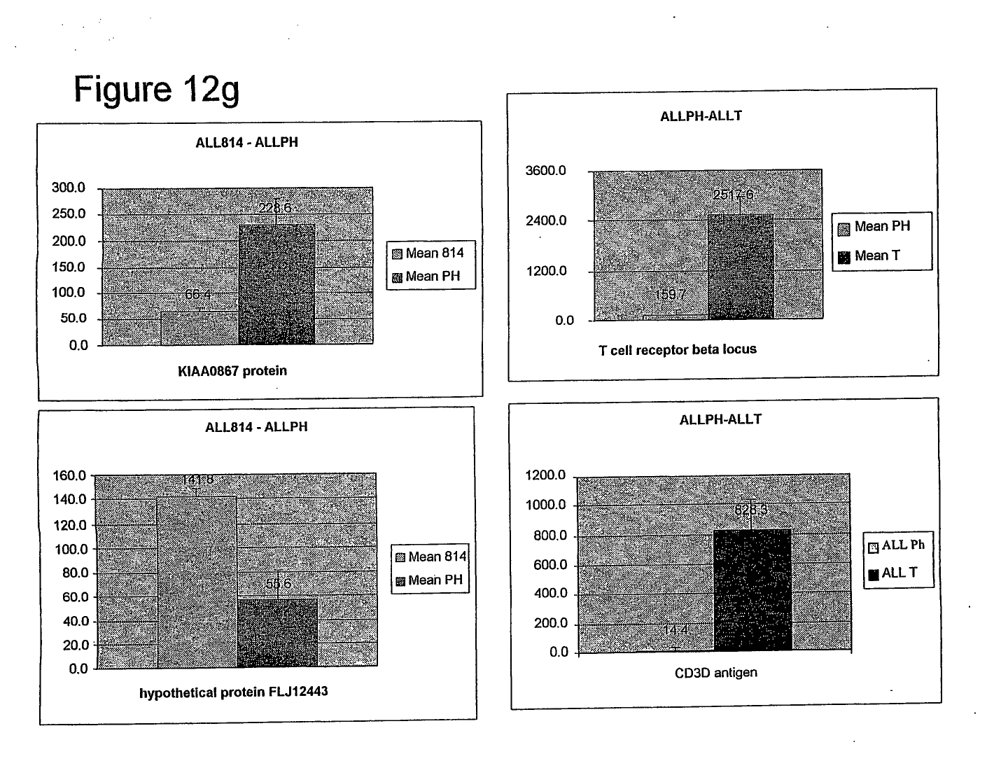

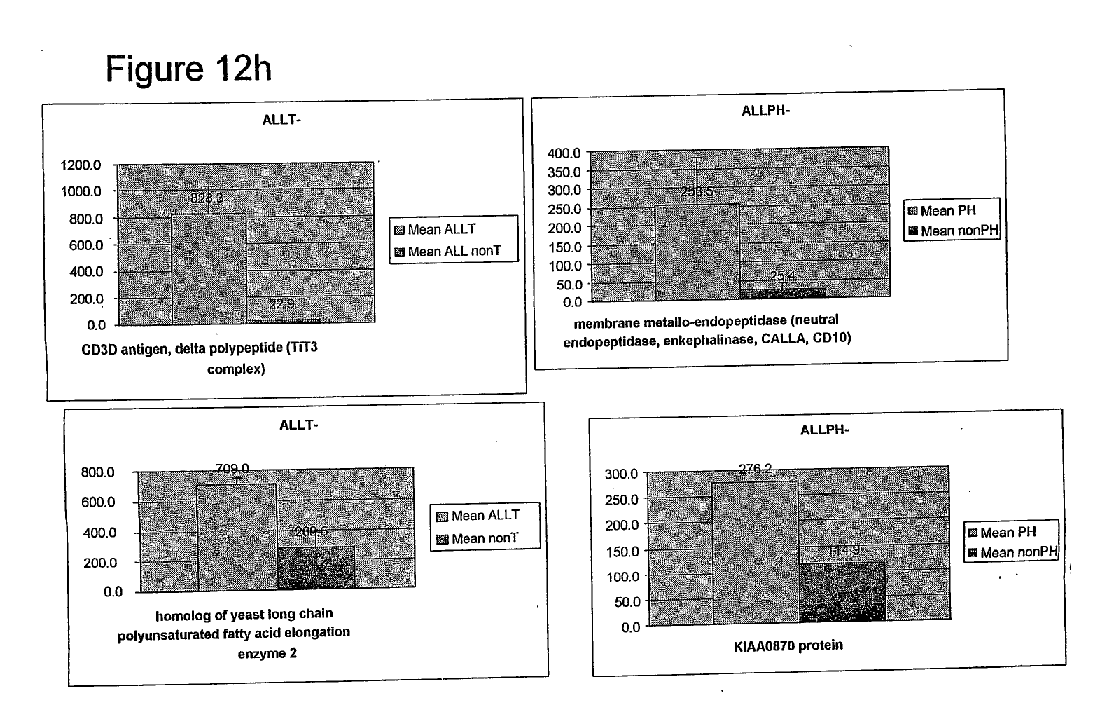

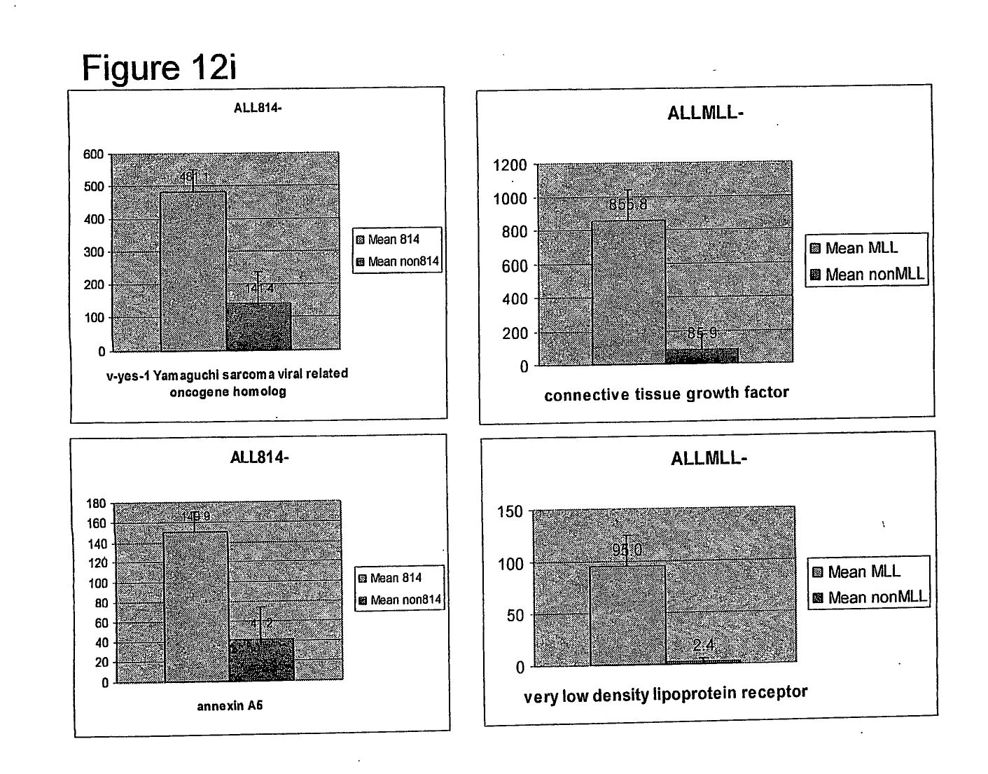

Particularly preferred markers used in a method where only one or a few as e.g. one, preferably two markers are used are described in Table 22 and Example 3, FIG. 12 or the markers marked with an asterisk in table 20 and shown in tables 16 to 19 as the preferred set of markers. In detail, example 3 mentions (see example 3 for more details) the following markers including their expression level:
ADCY3
adenosine deaminase (ADA)
ARGHGAP4
B-cell specific coactivator of octamer binding transcription factors
CAPN3 is a member of the papain superfamily and was higher expressed in CML
CBFβ-MYH11
CD24
CD27, was identified to assign samples either ALL or CLL
CD74 plays a critical role in MHC class II antigen processing
connective tissue growth factor (CTGF)
CTGF
CTSW
MYH11
glucocorticoid receptor beta
higher expression of CBFA2T1 (formerly ETO)
HLA-DMB
HOXA9
HOXB5
IRF4, an immune system-restricted interferon regulatory factor
KIAA1013
LCN2 that shown to be a modulator of inflammation
LEF-1 was absent in myeloid leukemias but highly expressed in lymphoid leukemias
MBNL
MSF translocation partner of the mixed-lineage leukemia gene (MLL) in AML
NCOA 1 expressed higher in CLL as compared to ALL
OS-9differentially expressed between AML and ALL (14)
Phospholipidscramblase 1 (PLSCR1) to be lower expressed in AML and ALL as compared to normal BM
POU2AF1
POU2F2
POU4F1
SCYA3
SGP28
SOCS-2
TRB and CD3D

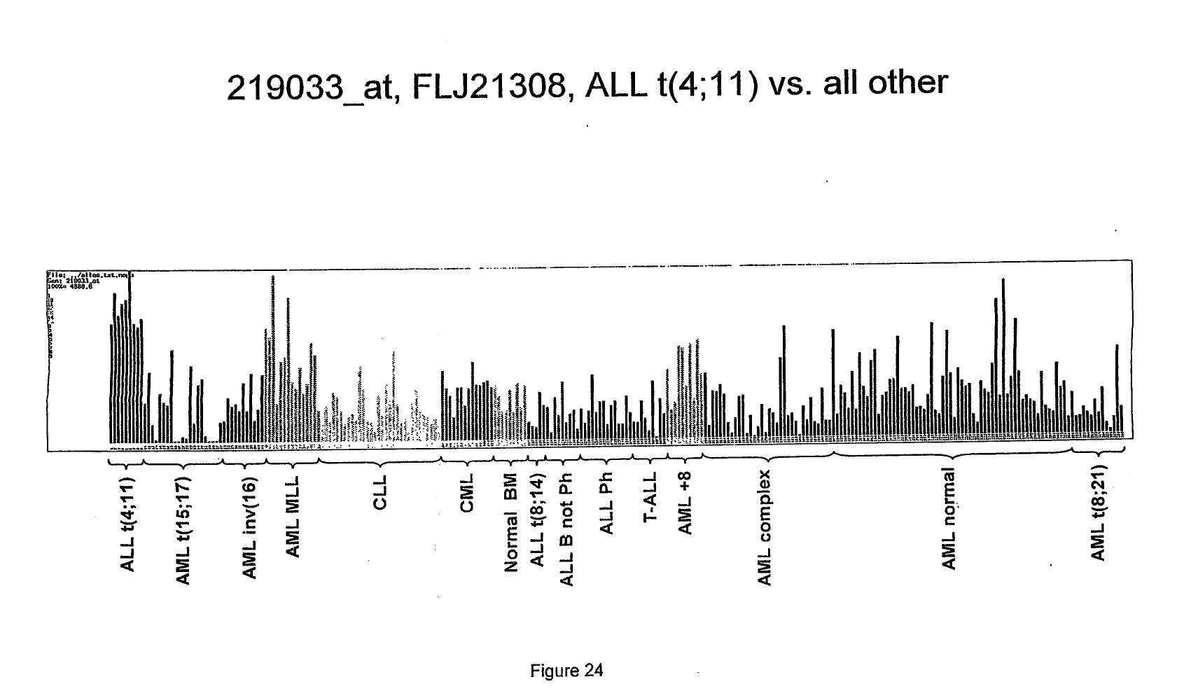

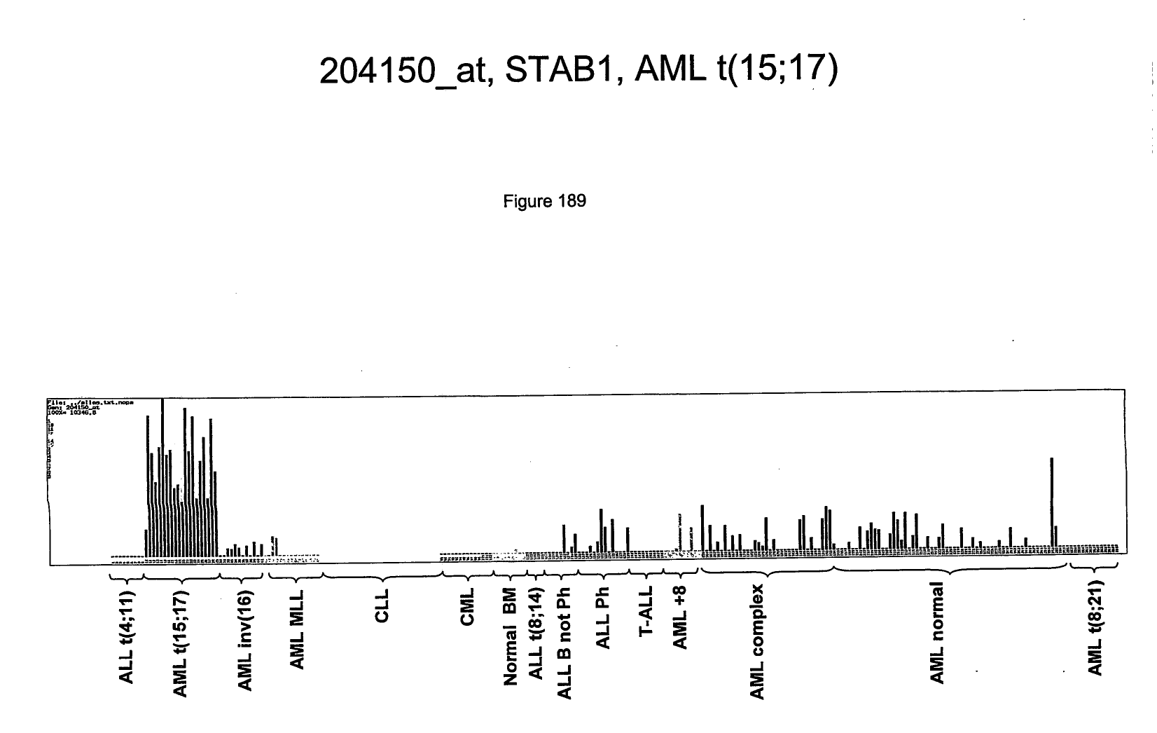

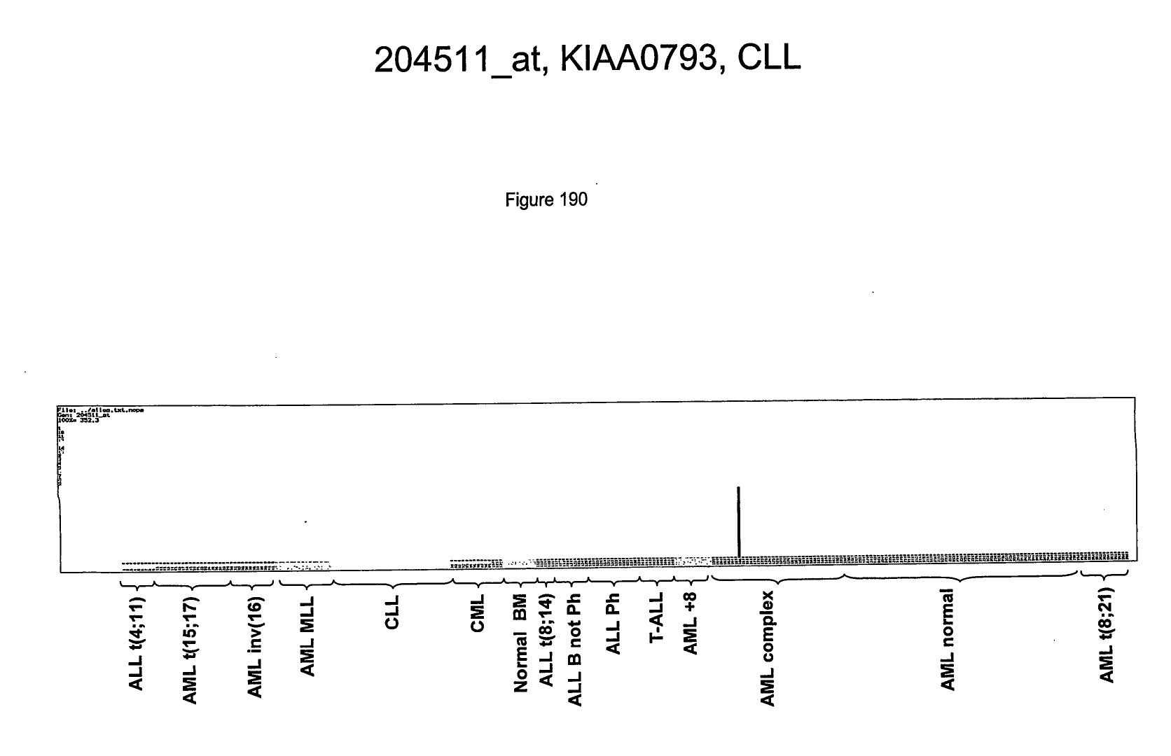

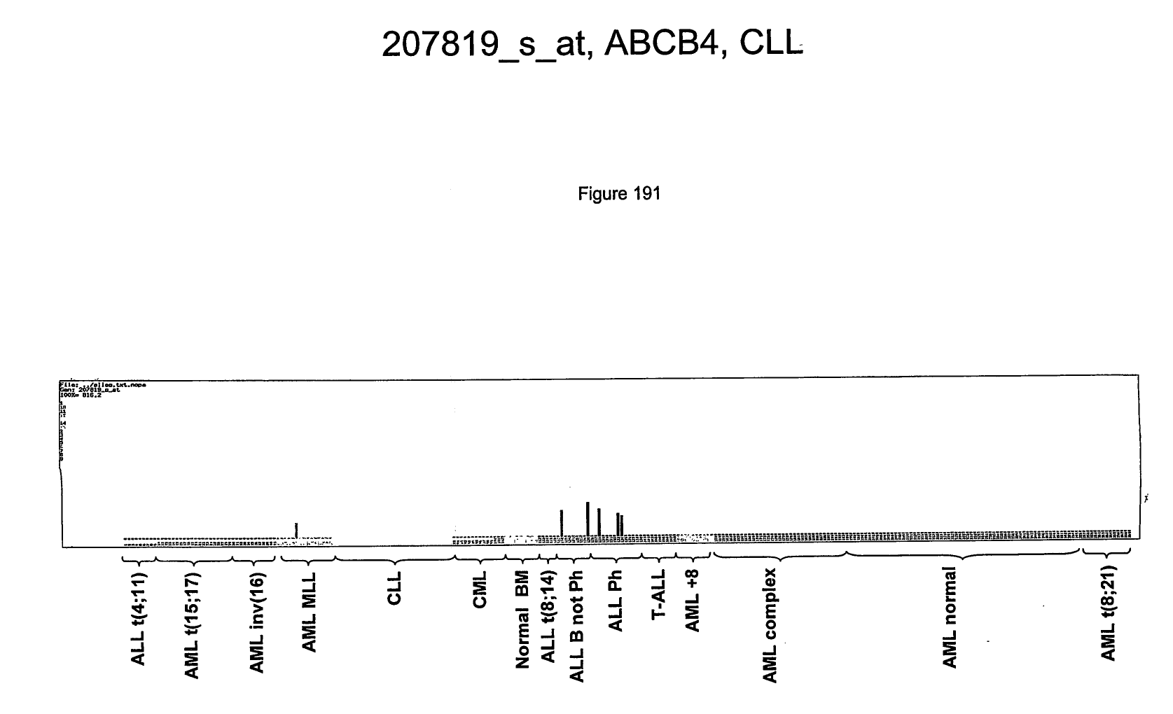

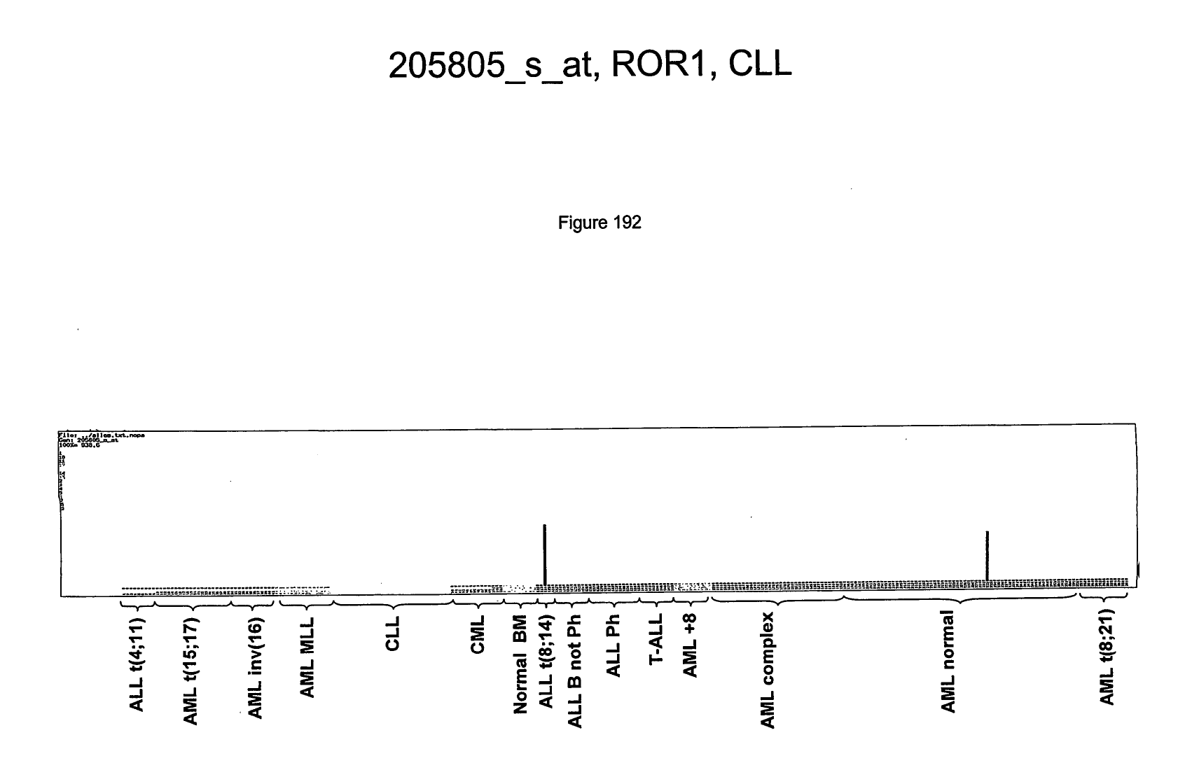

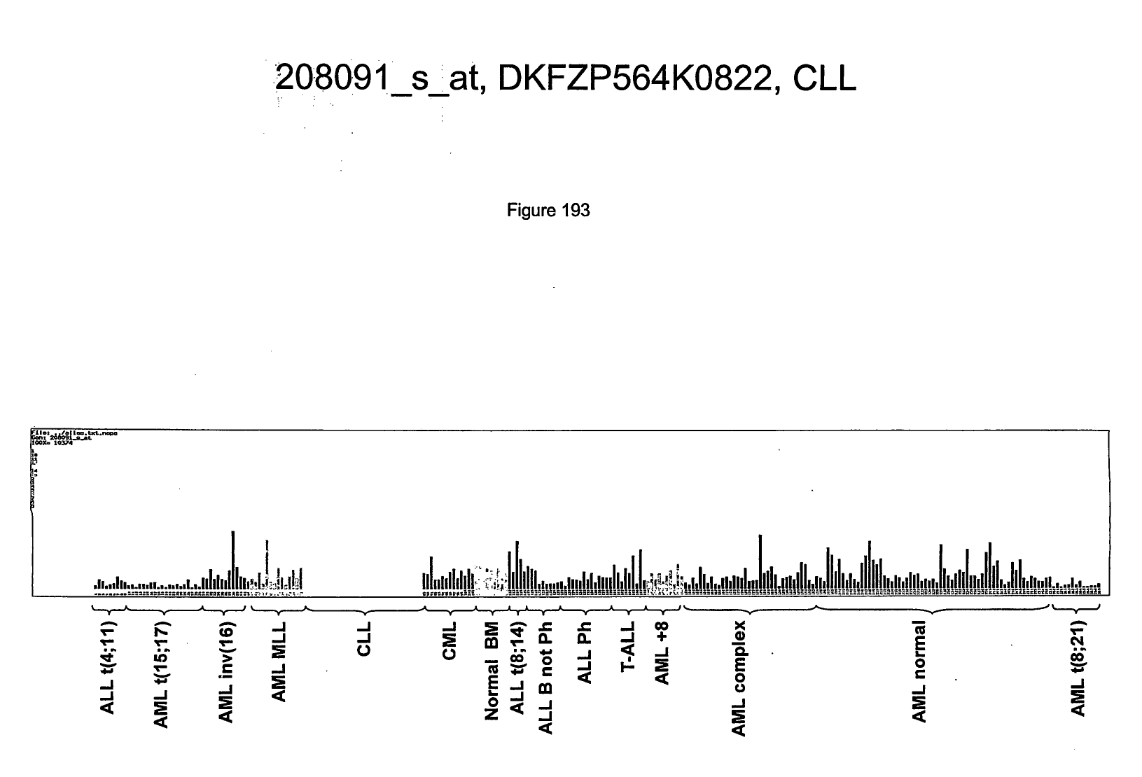

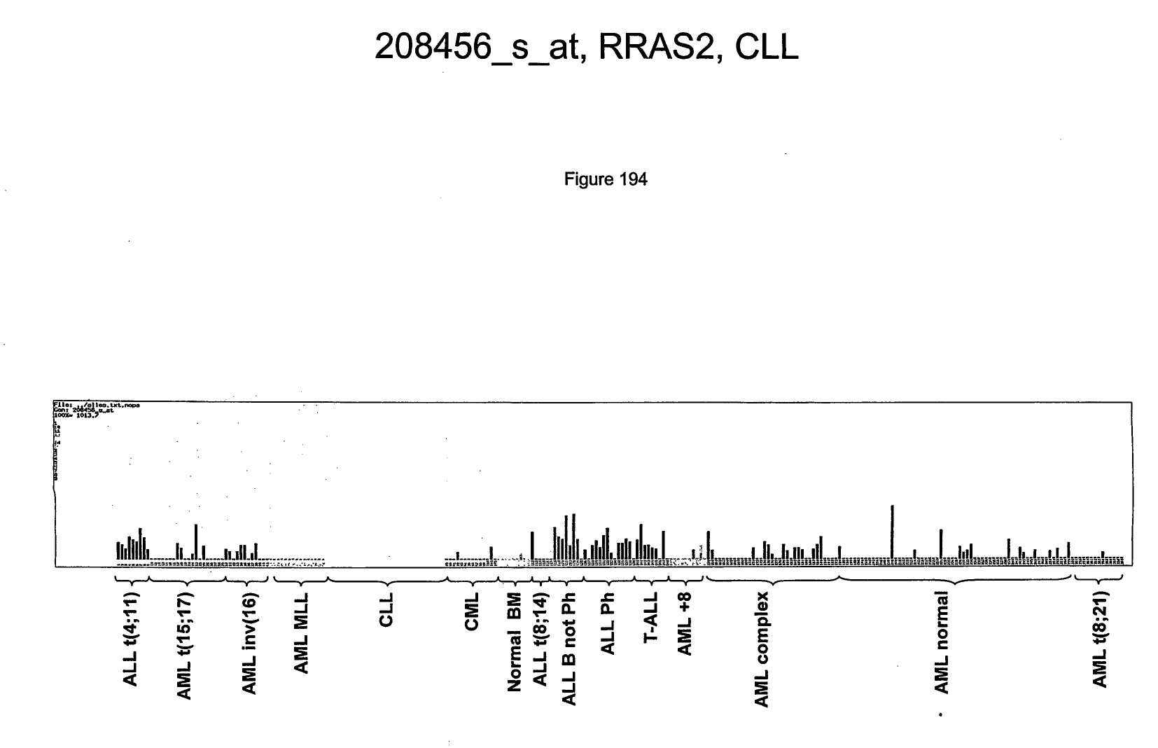

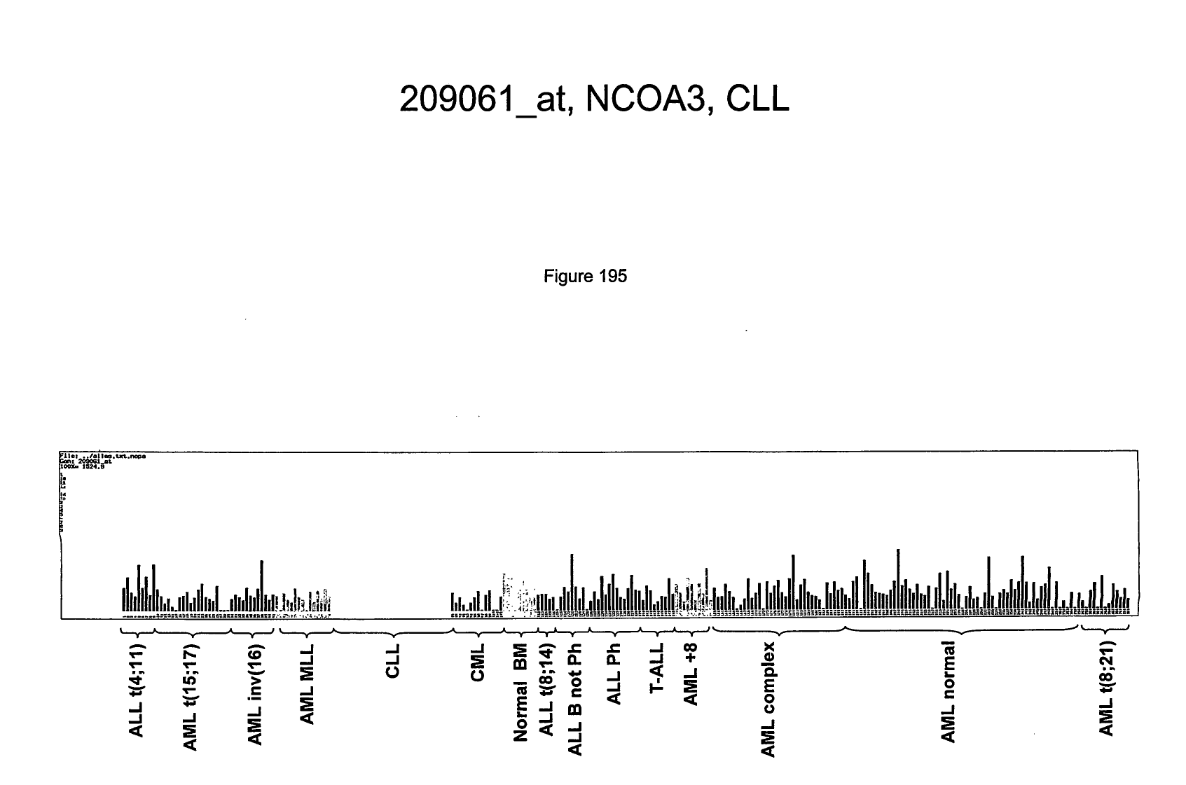

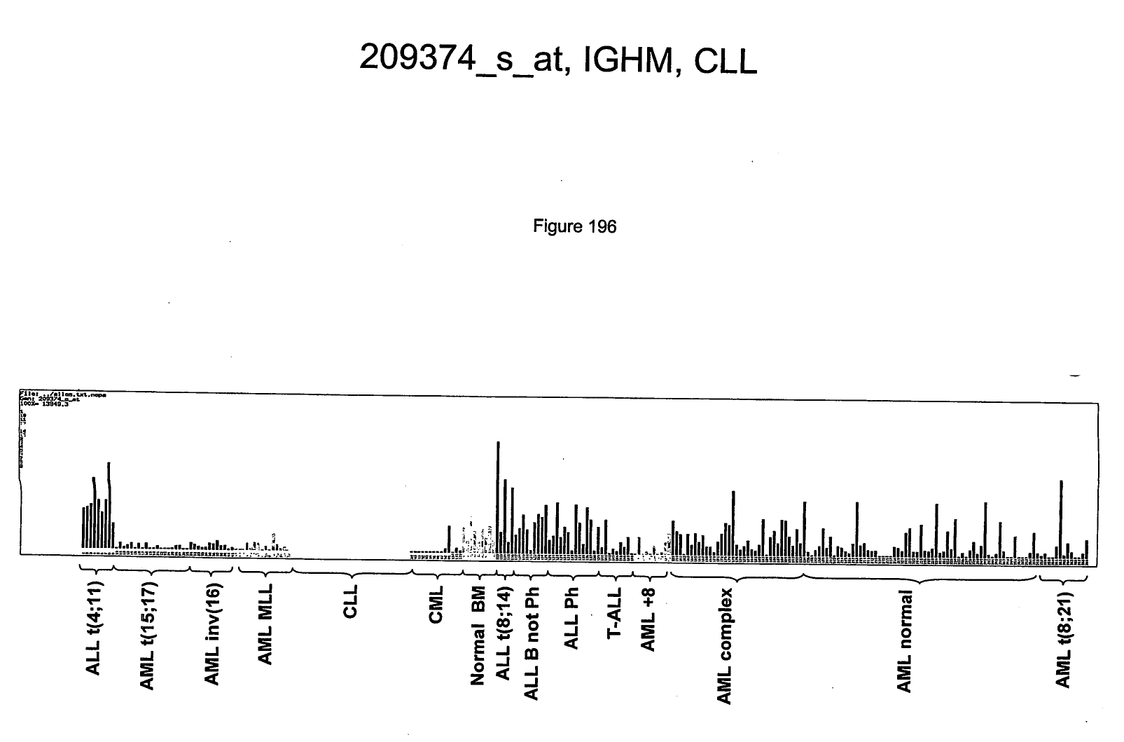

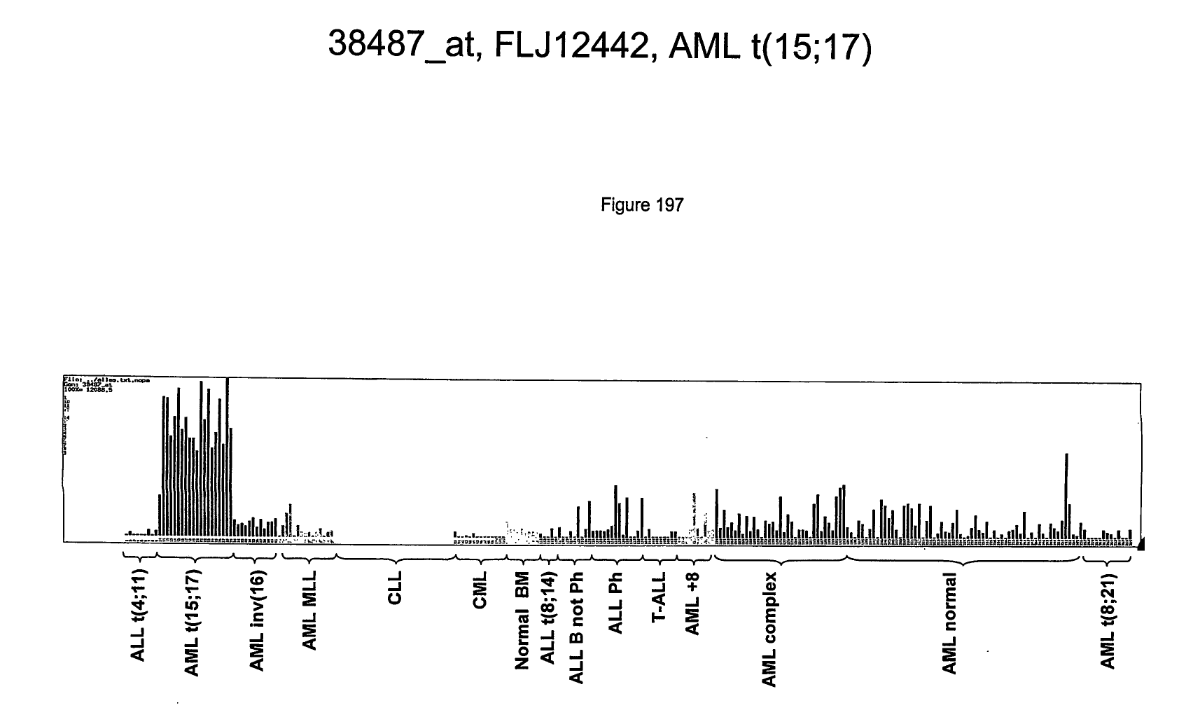

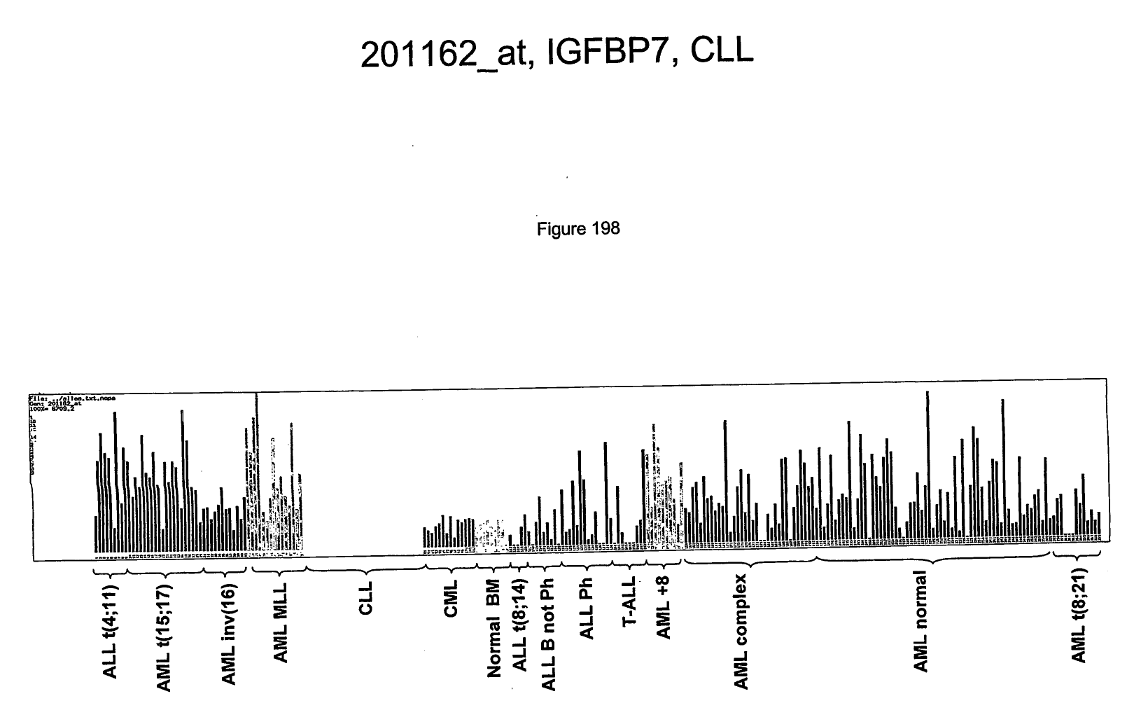

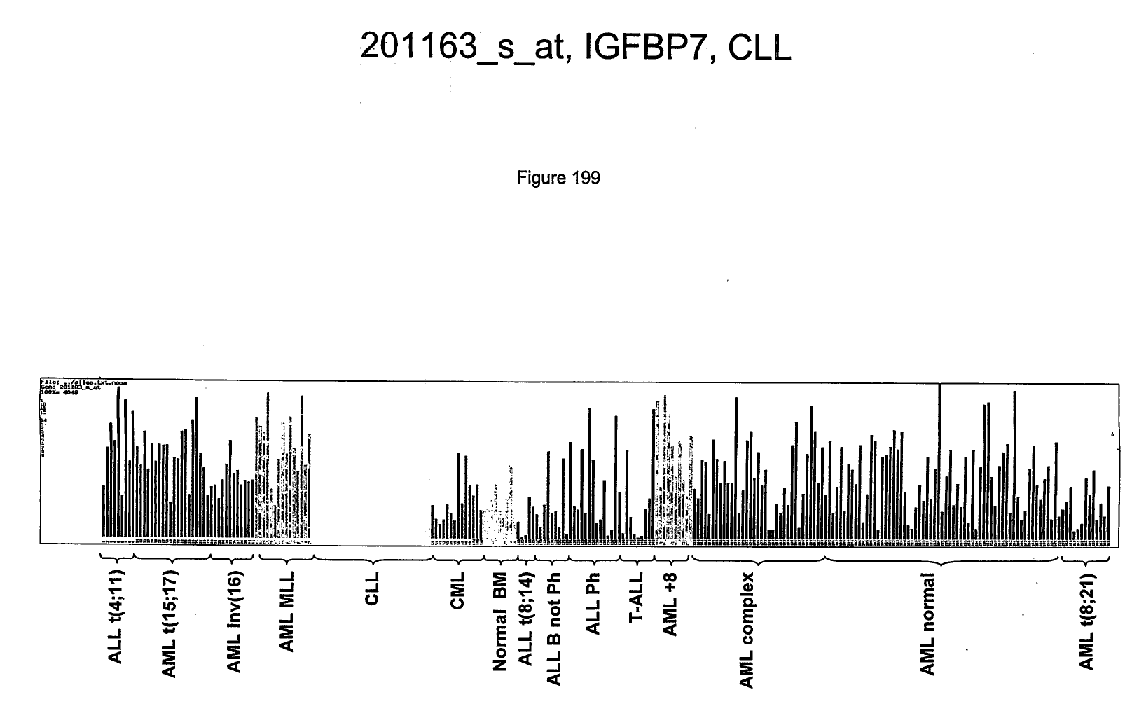

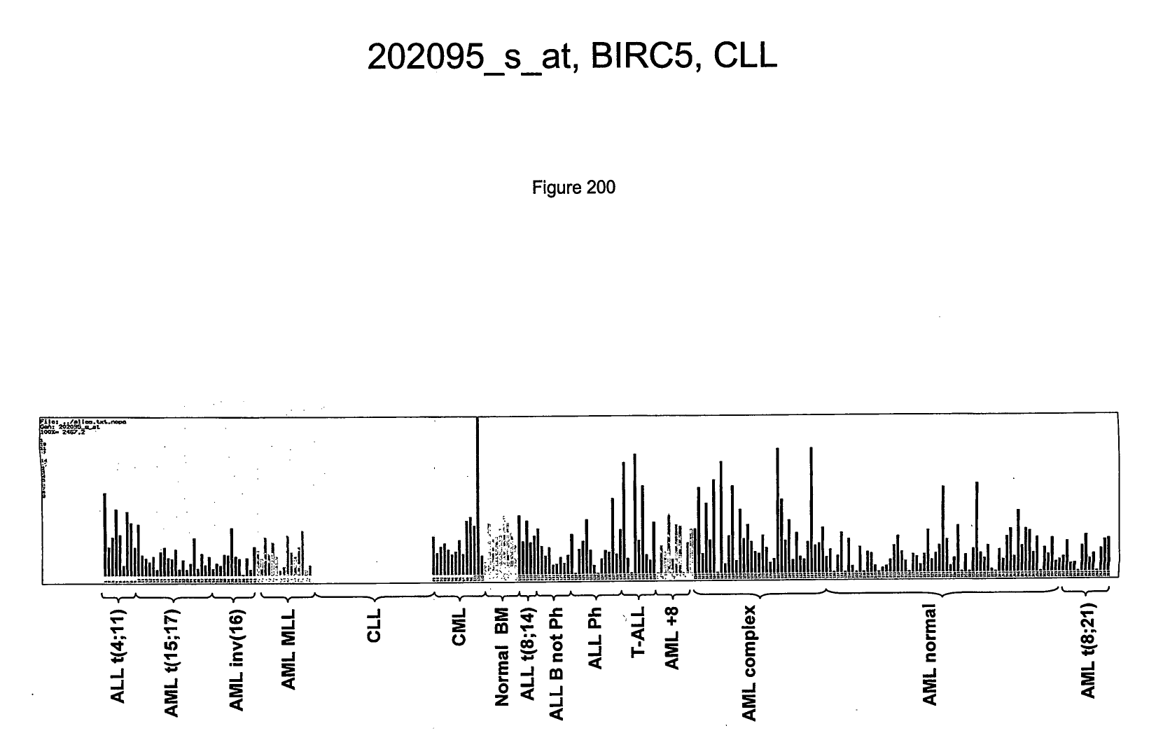

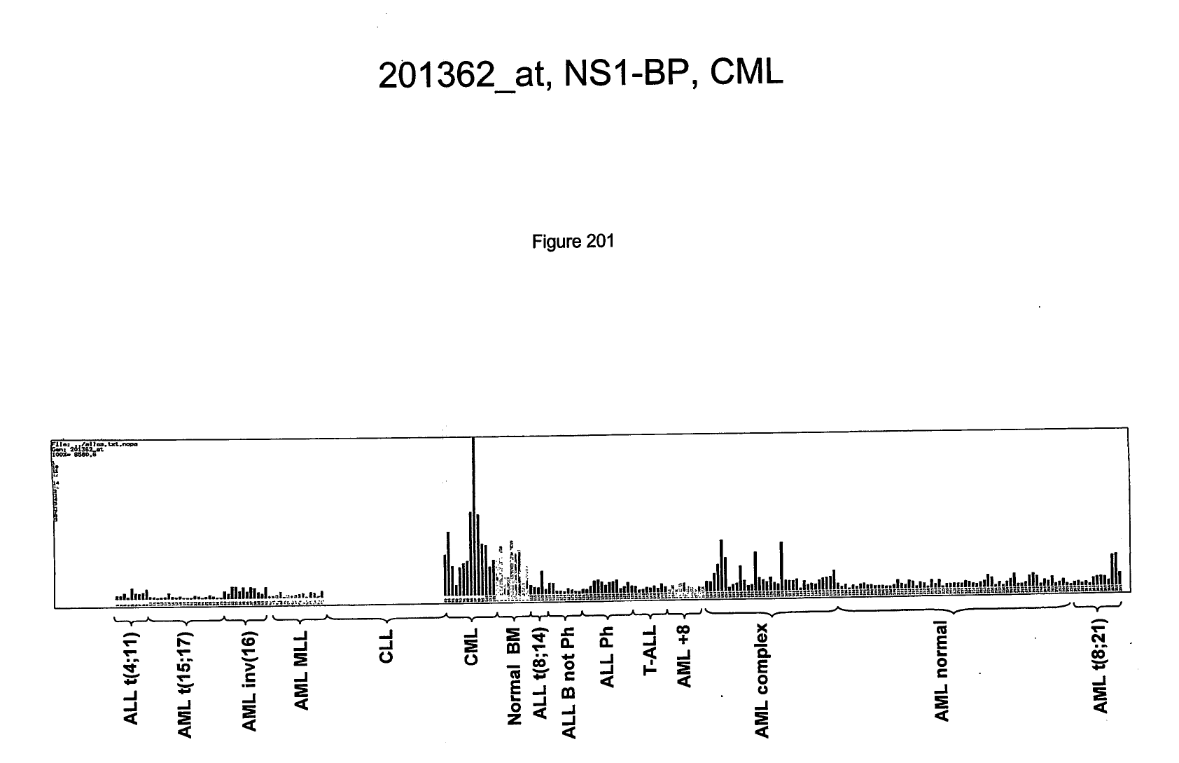

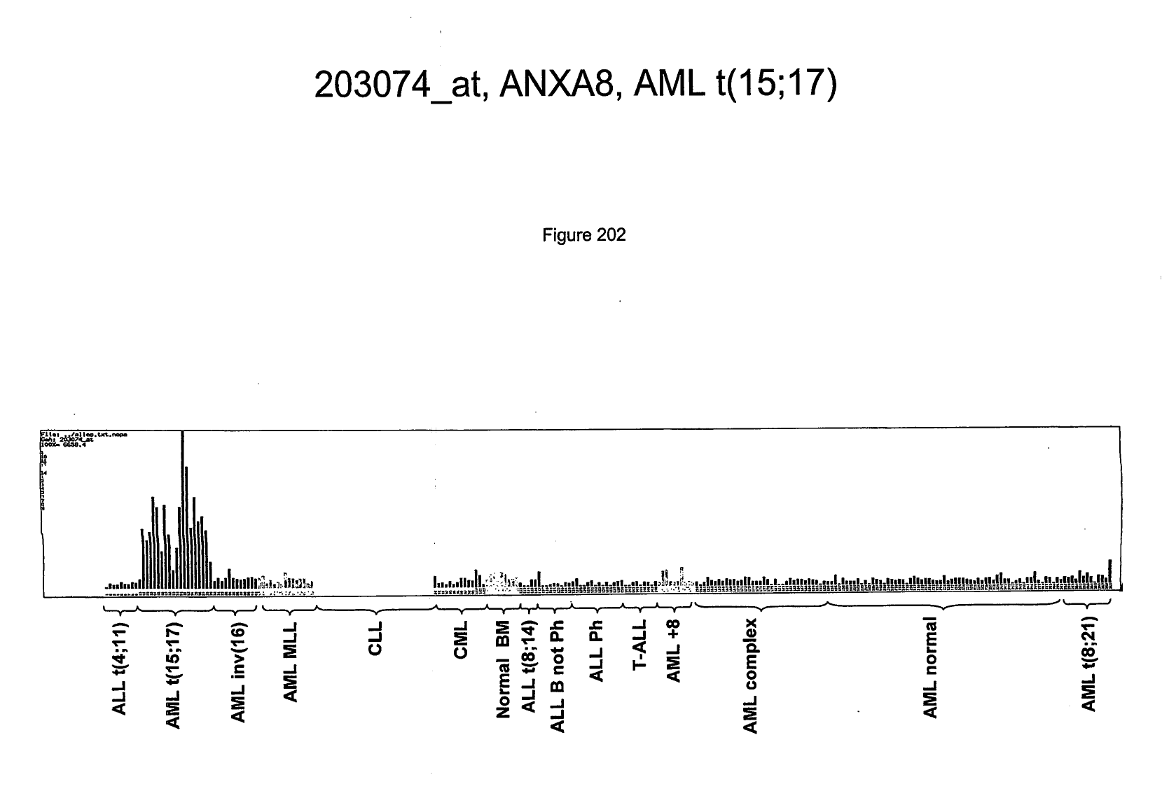

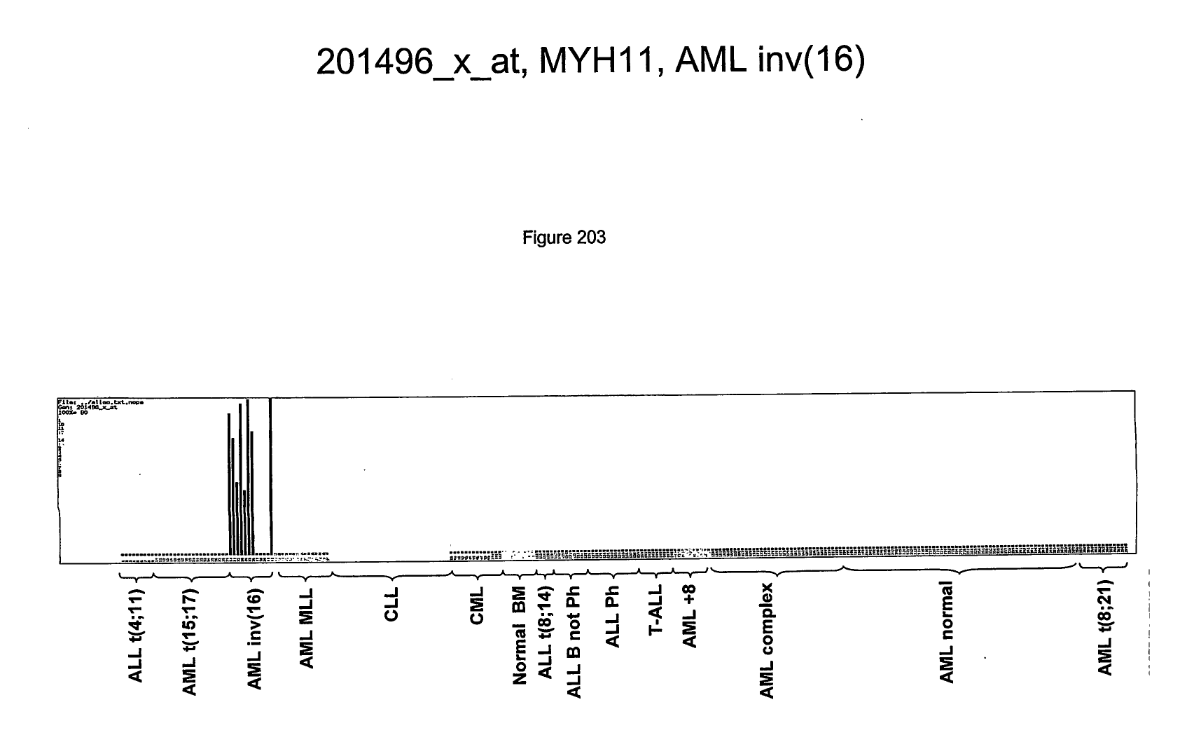

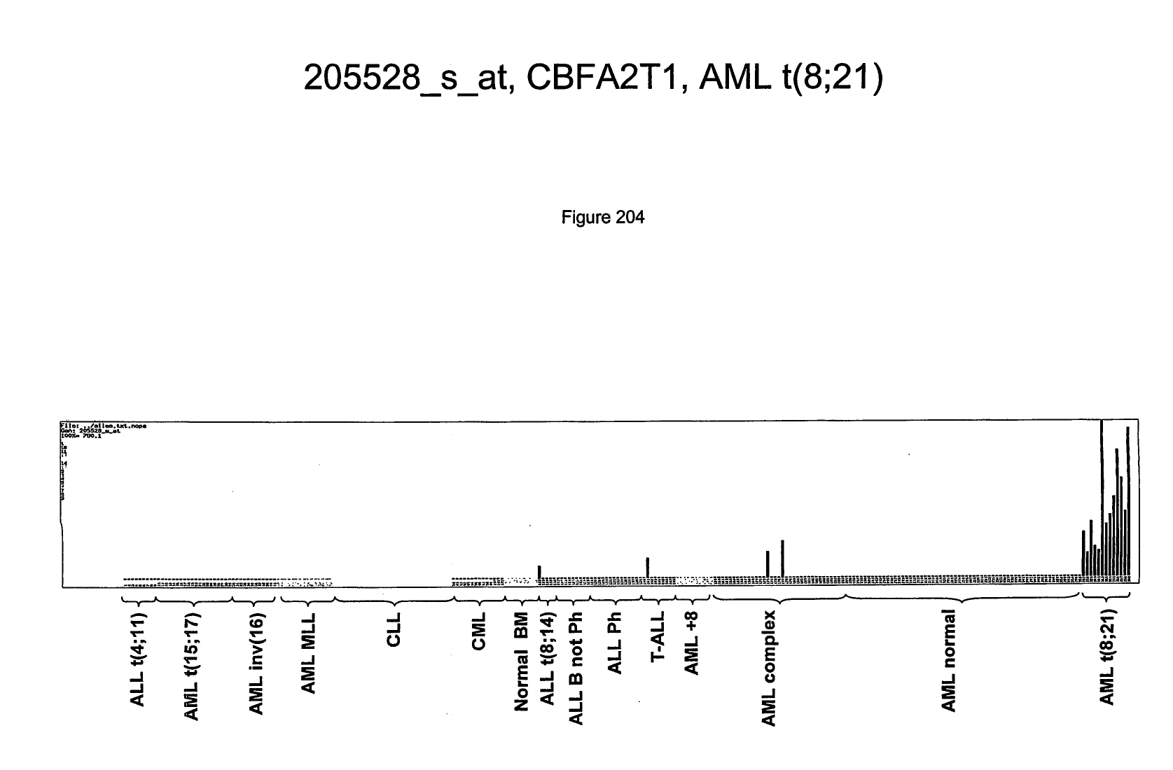

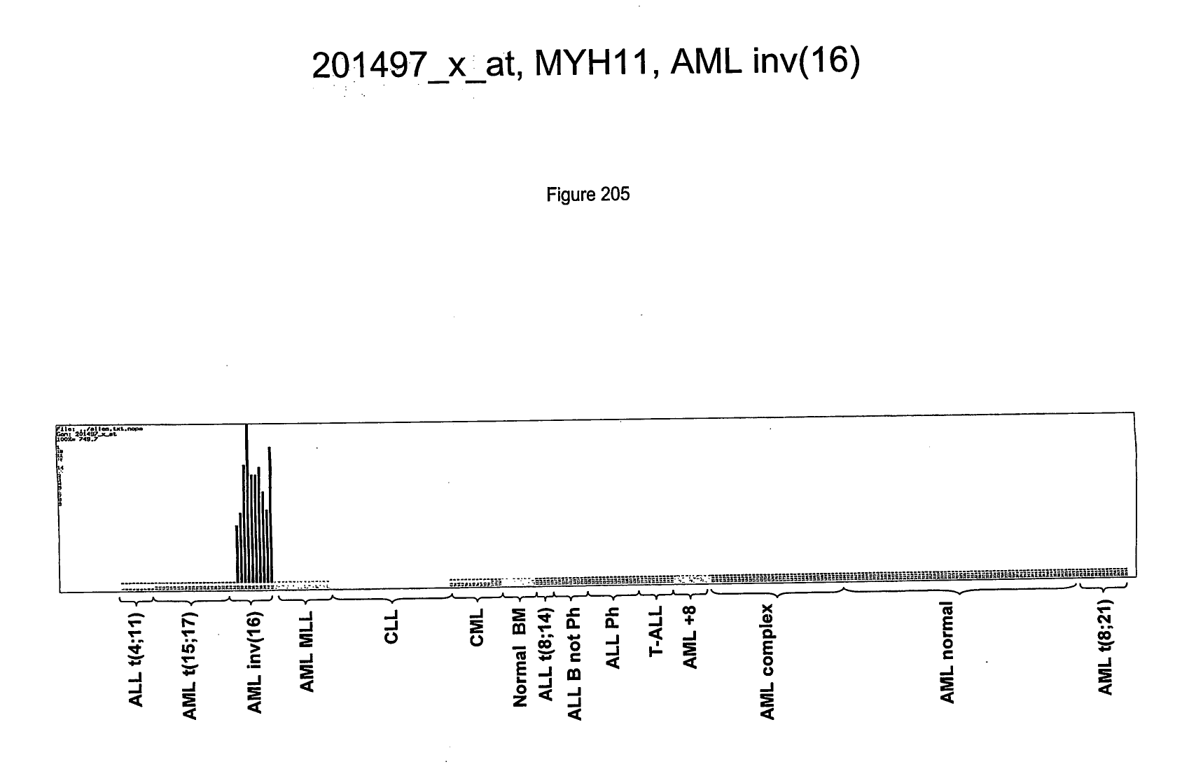

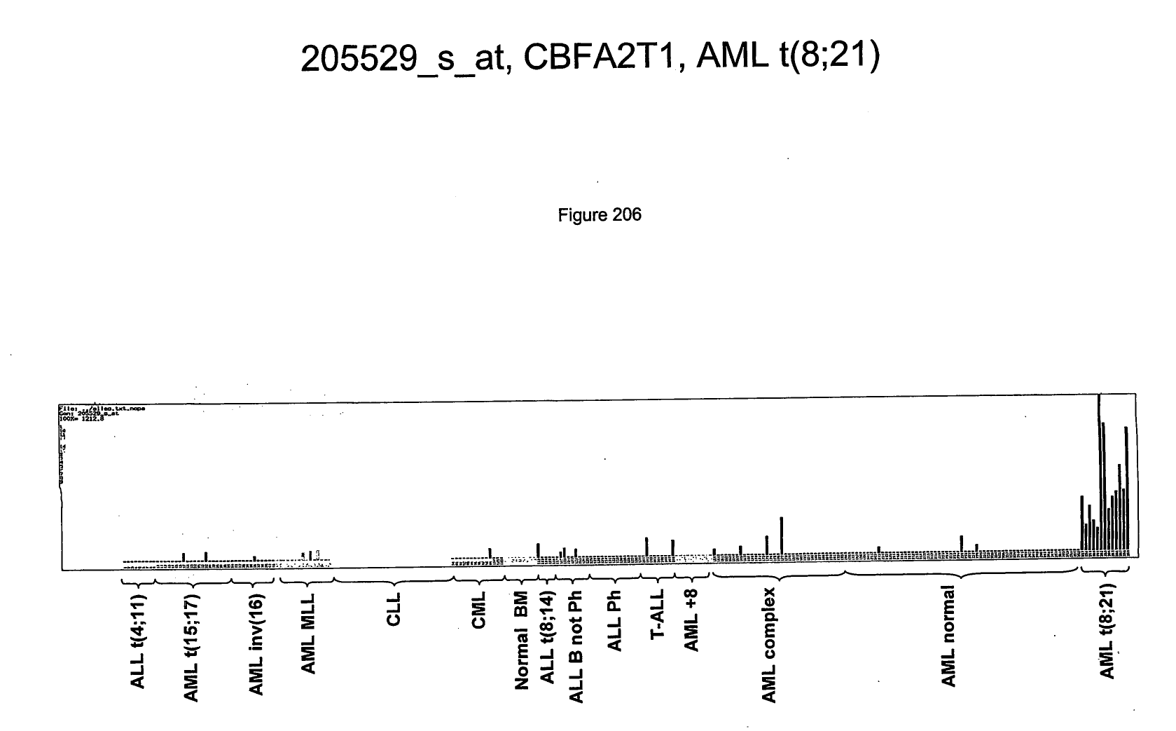

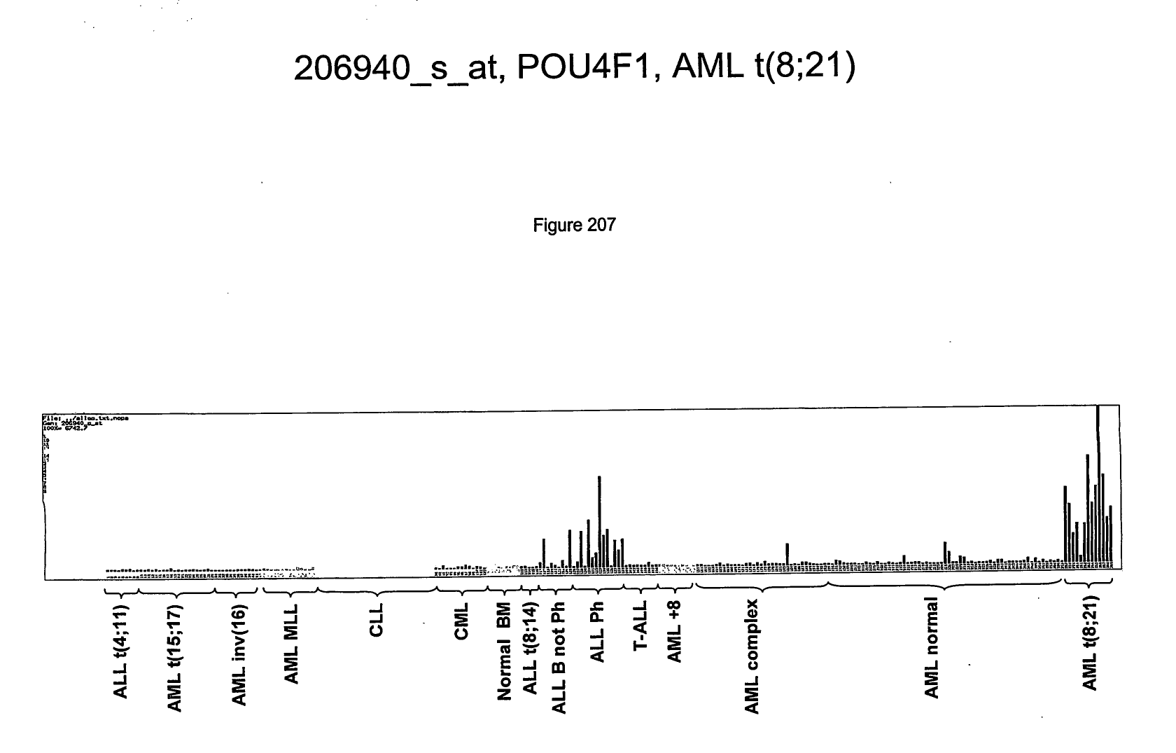

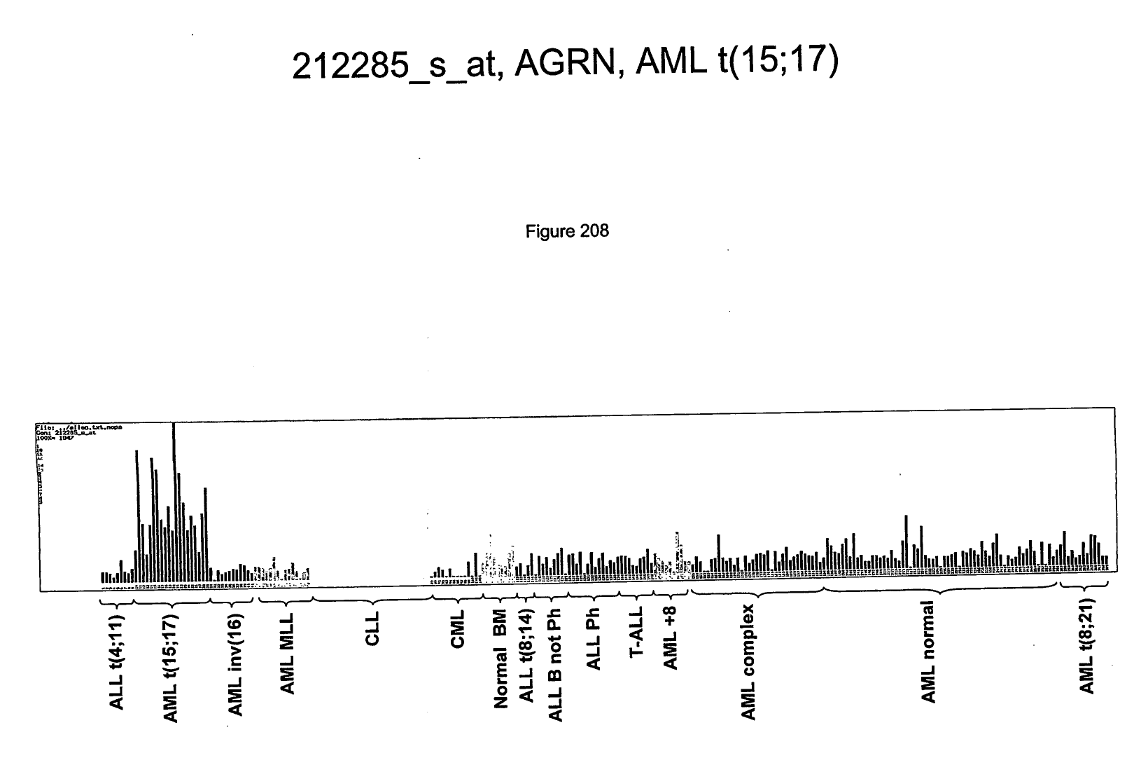

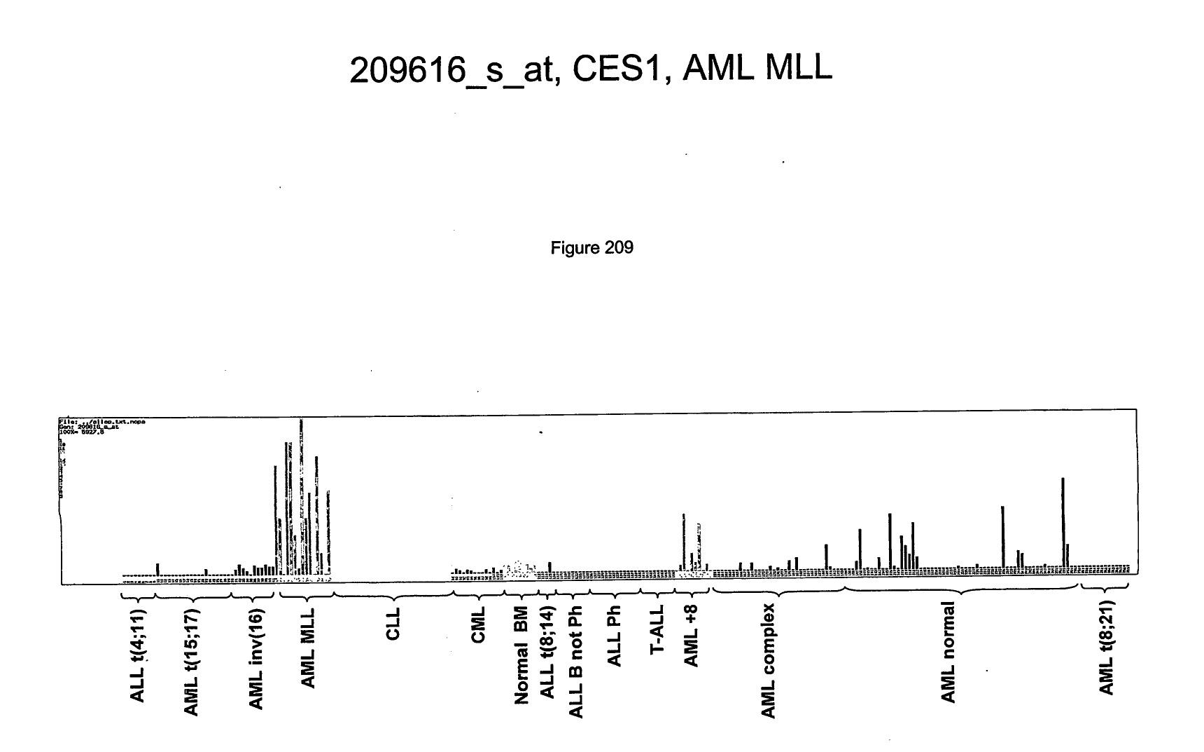

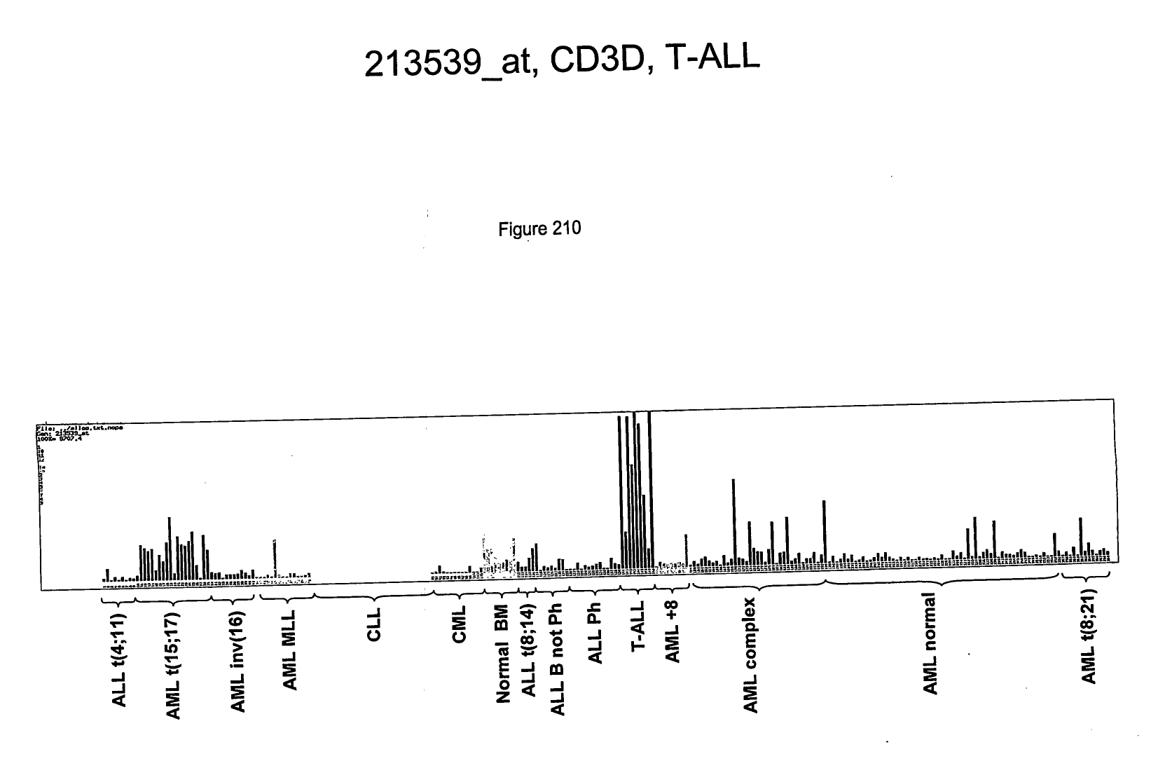

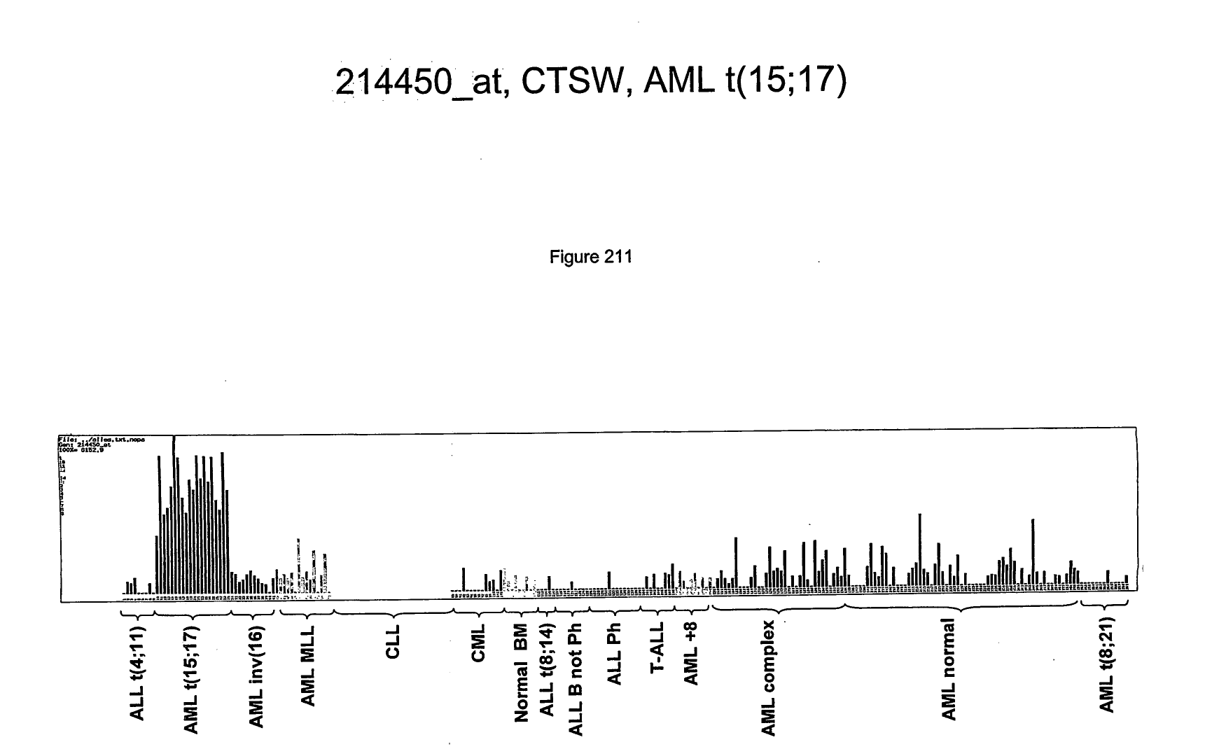

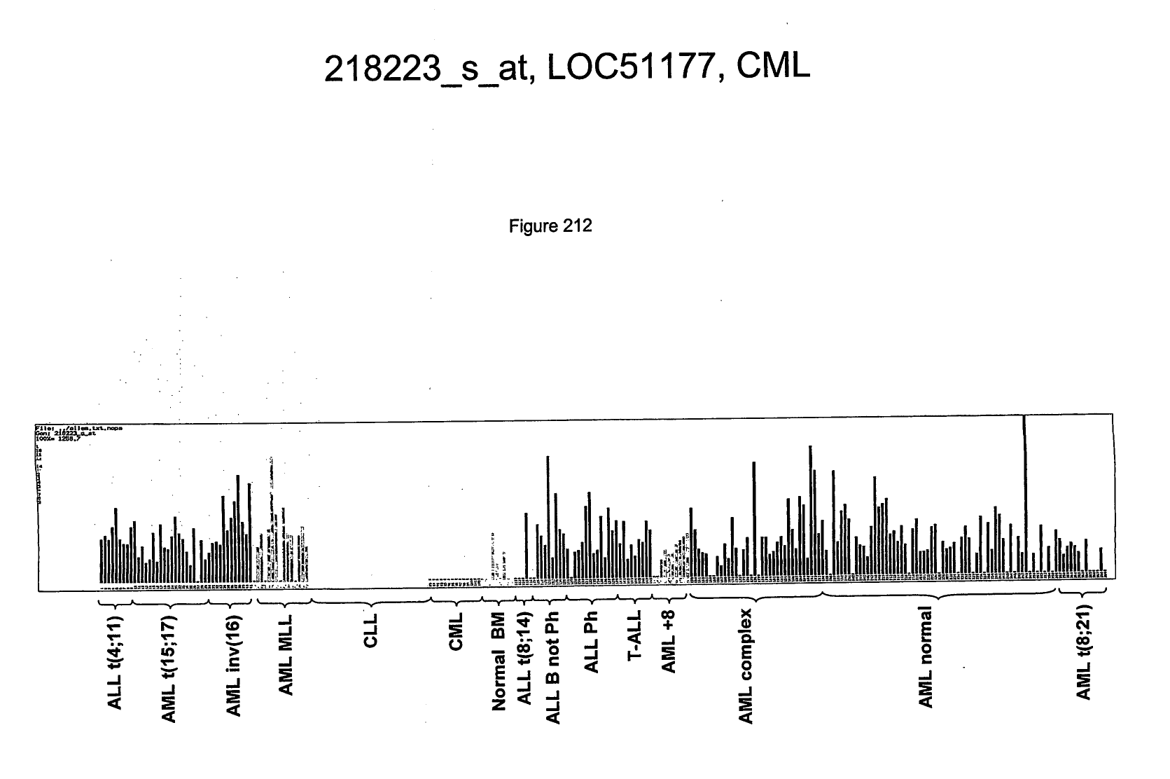

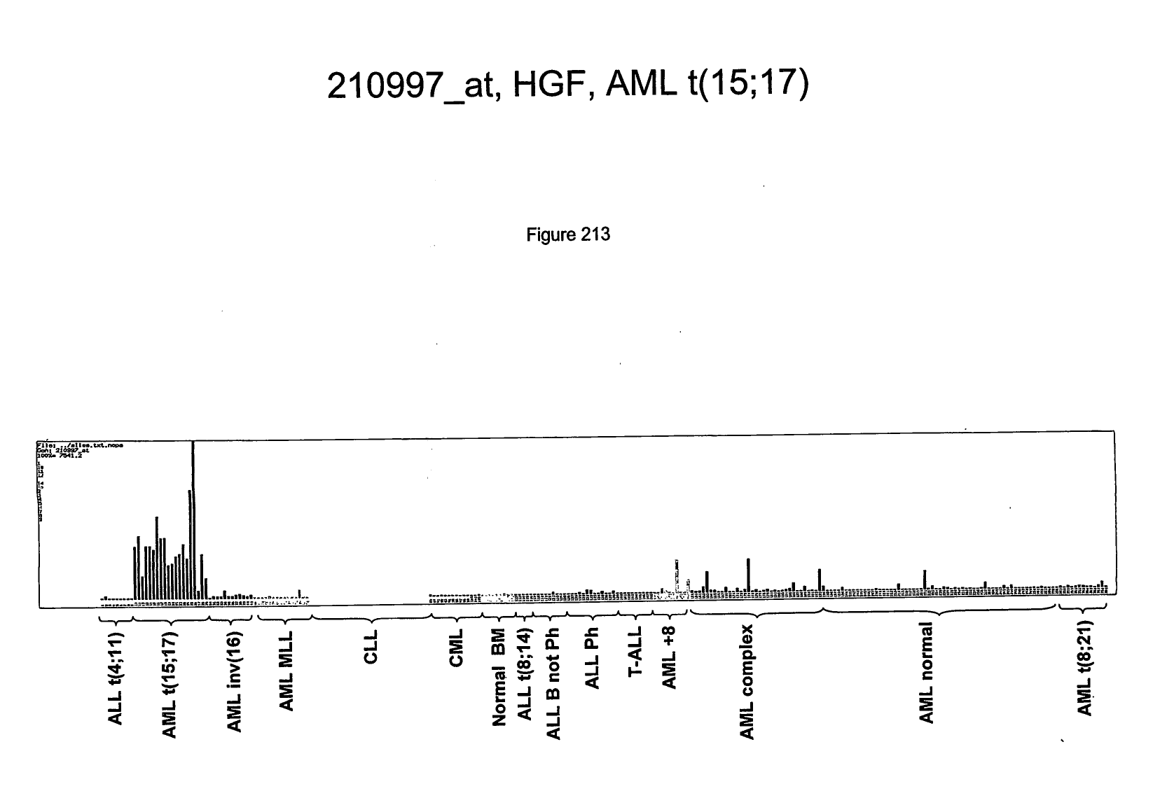

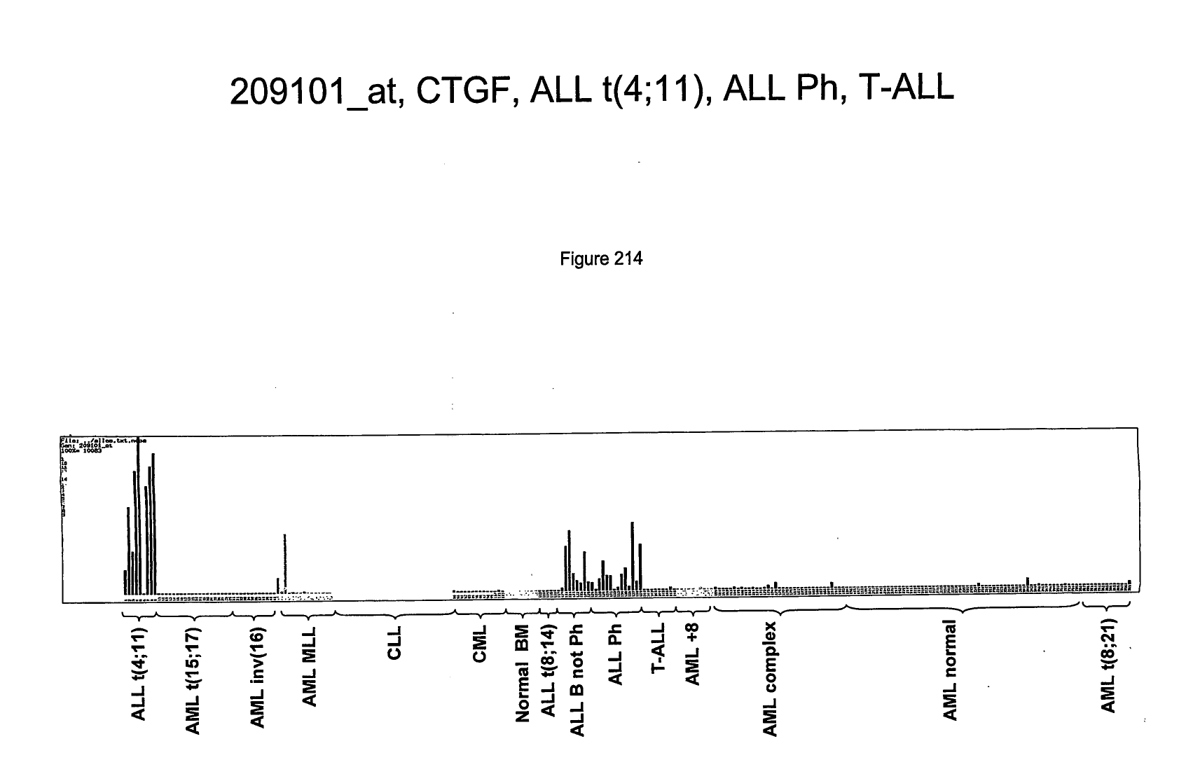

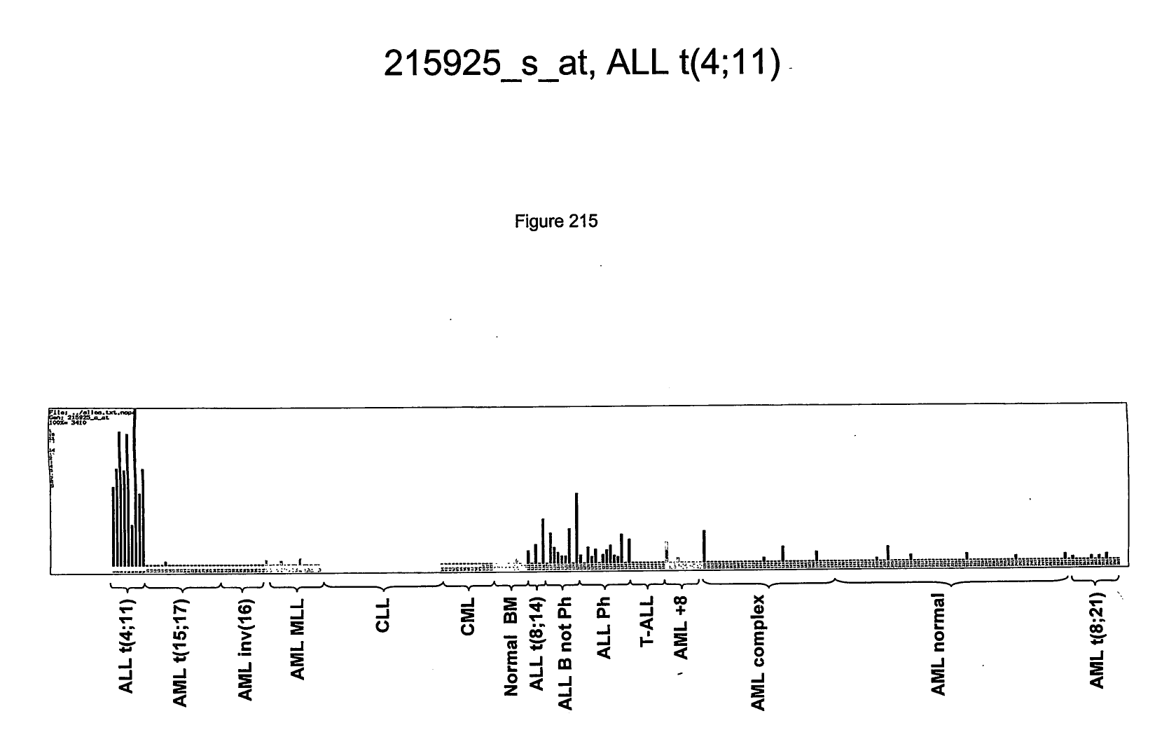

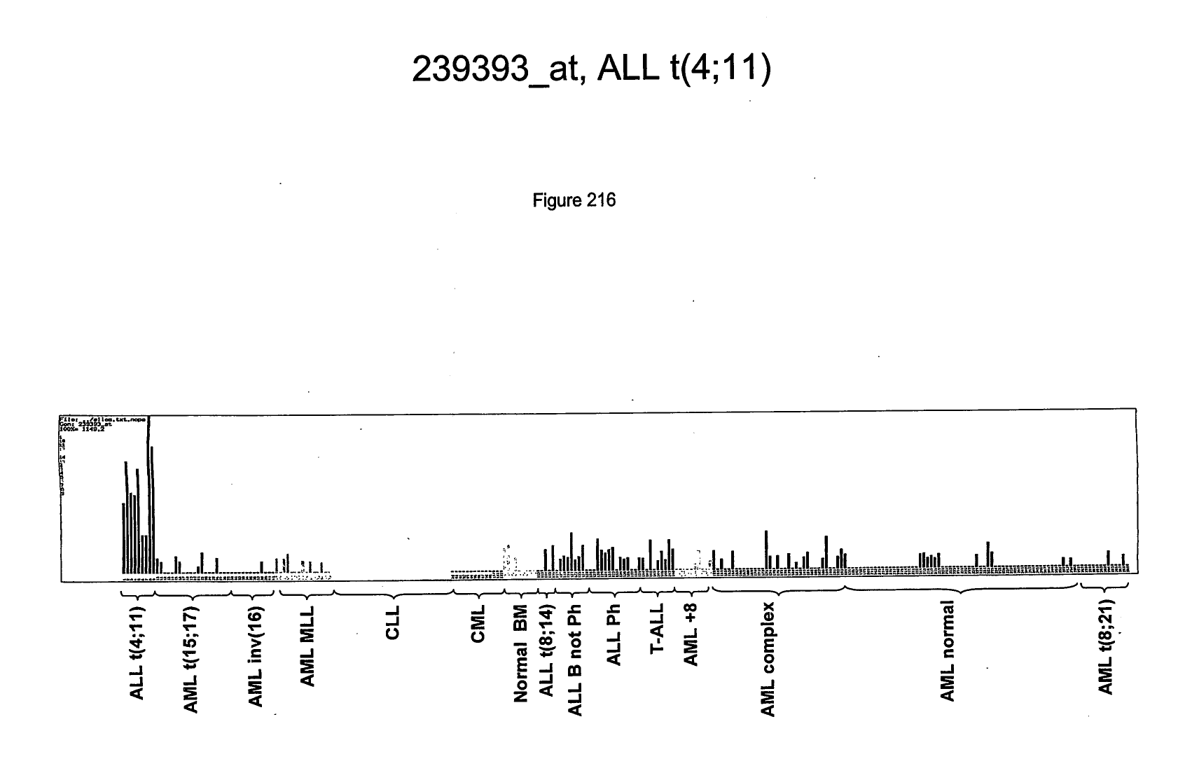

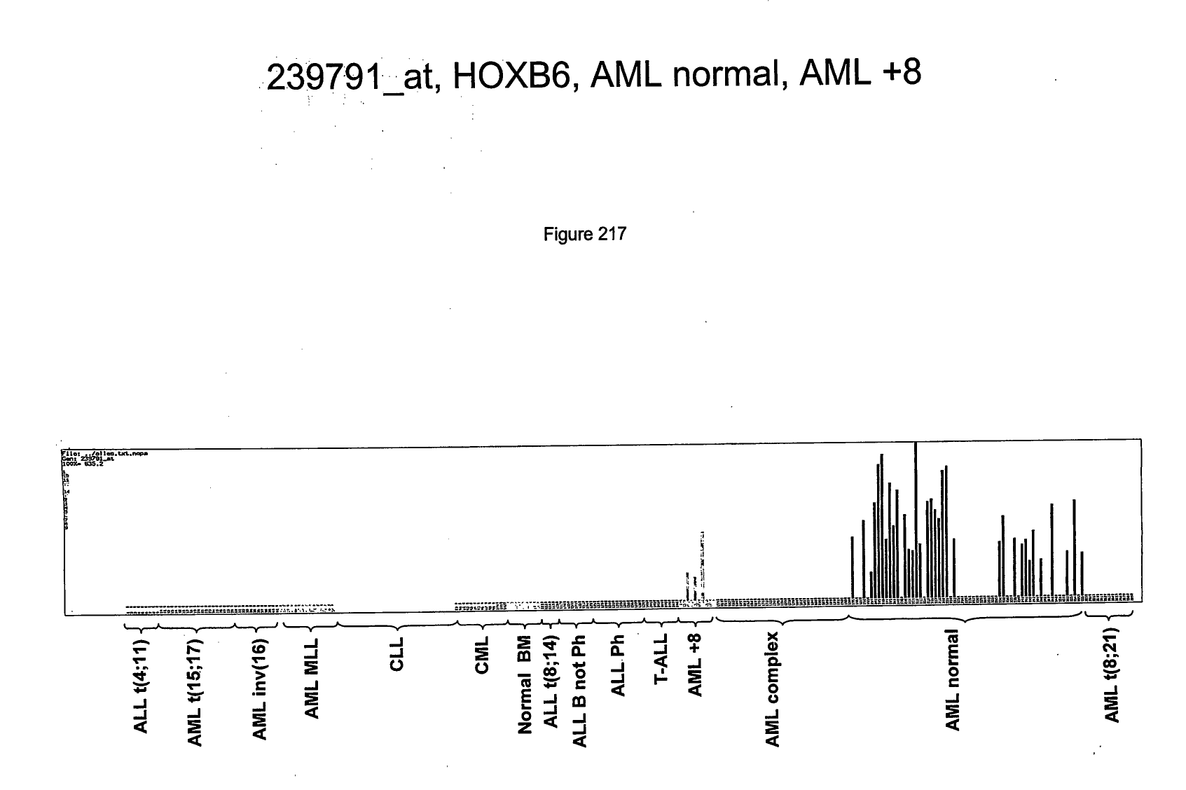

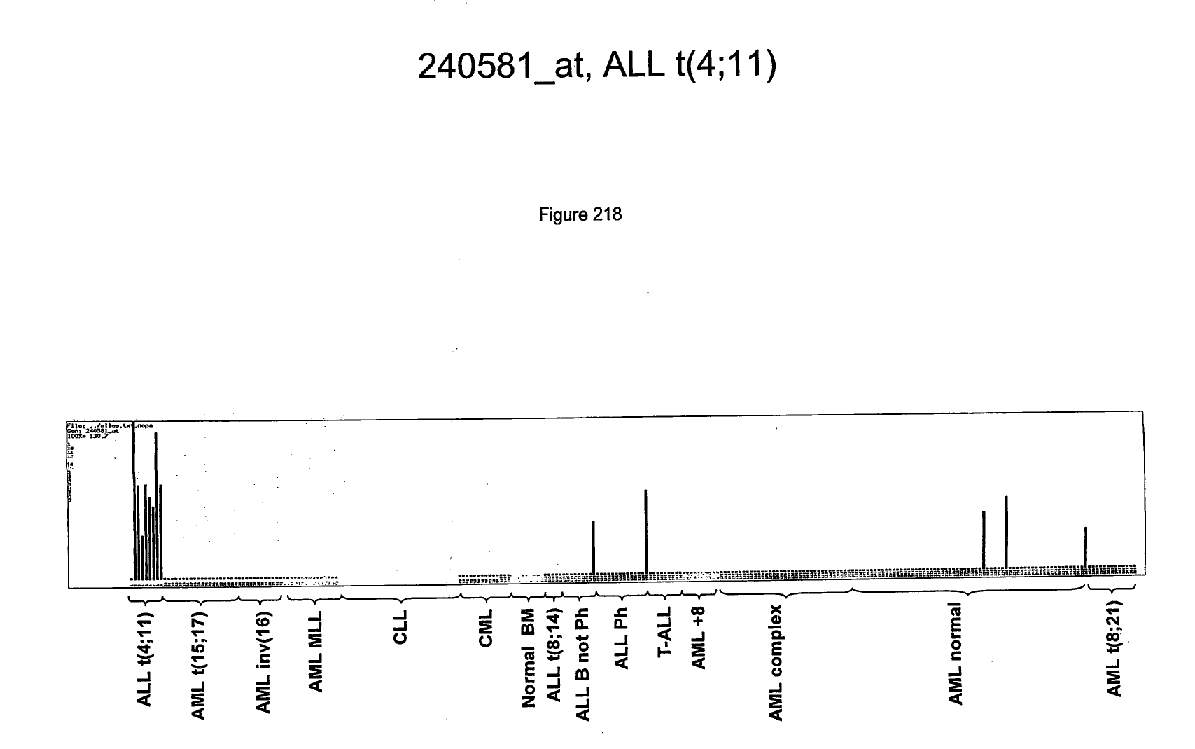

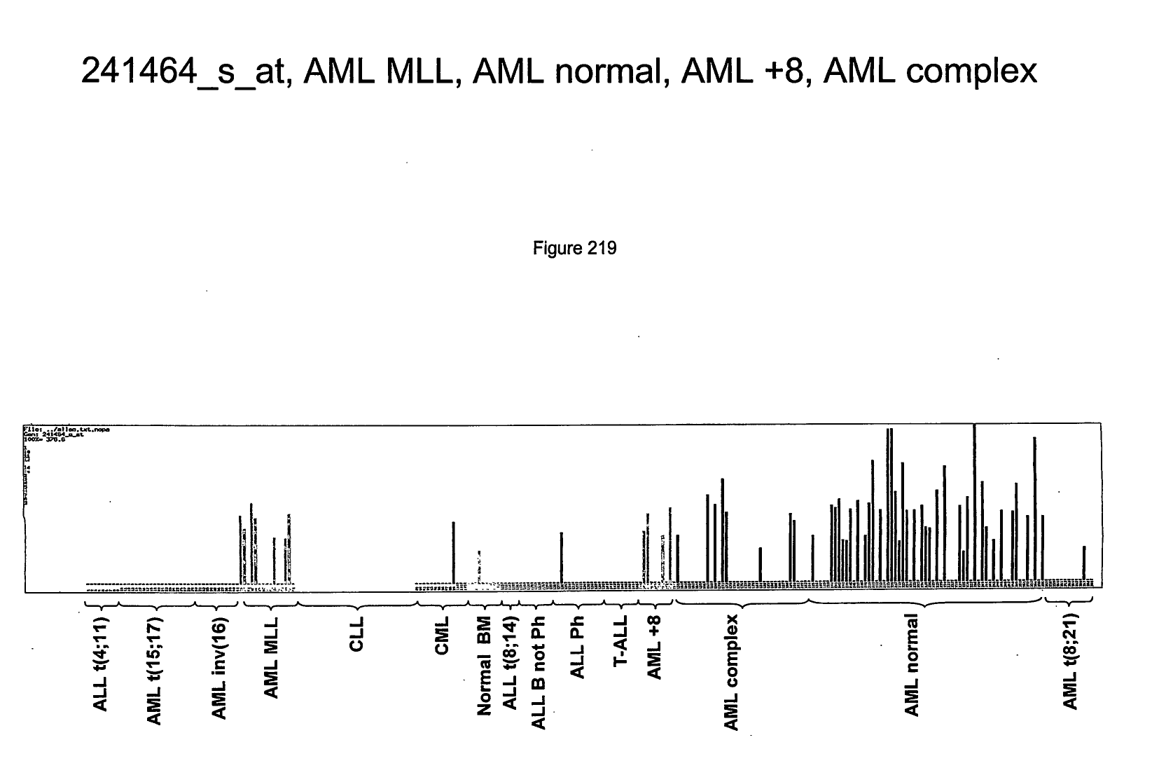

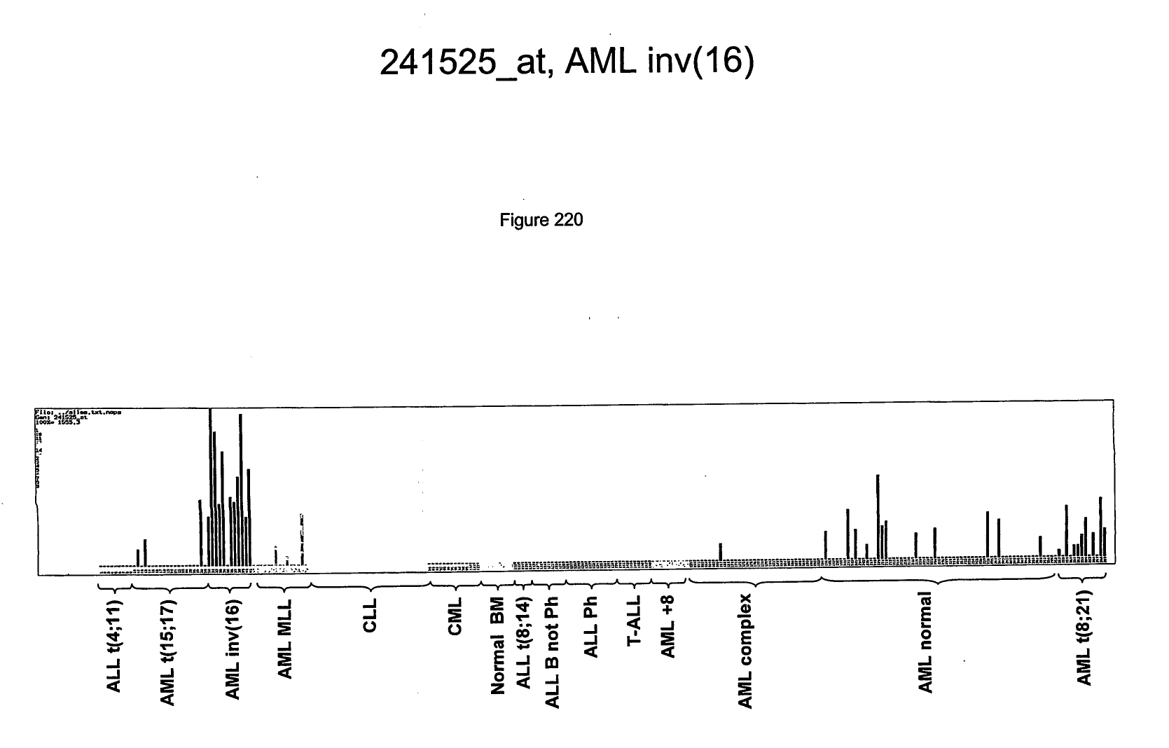

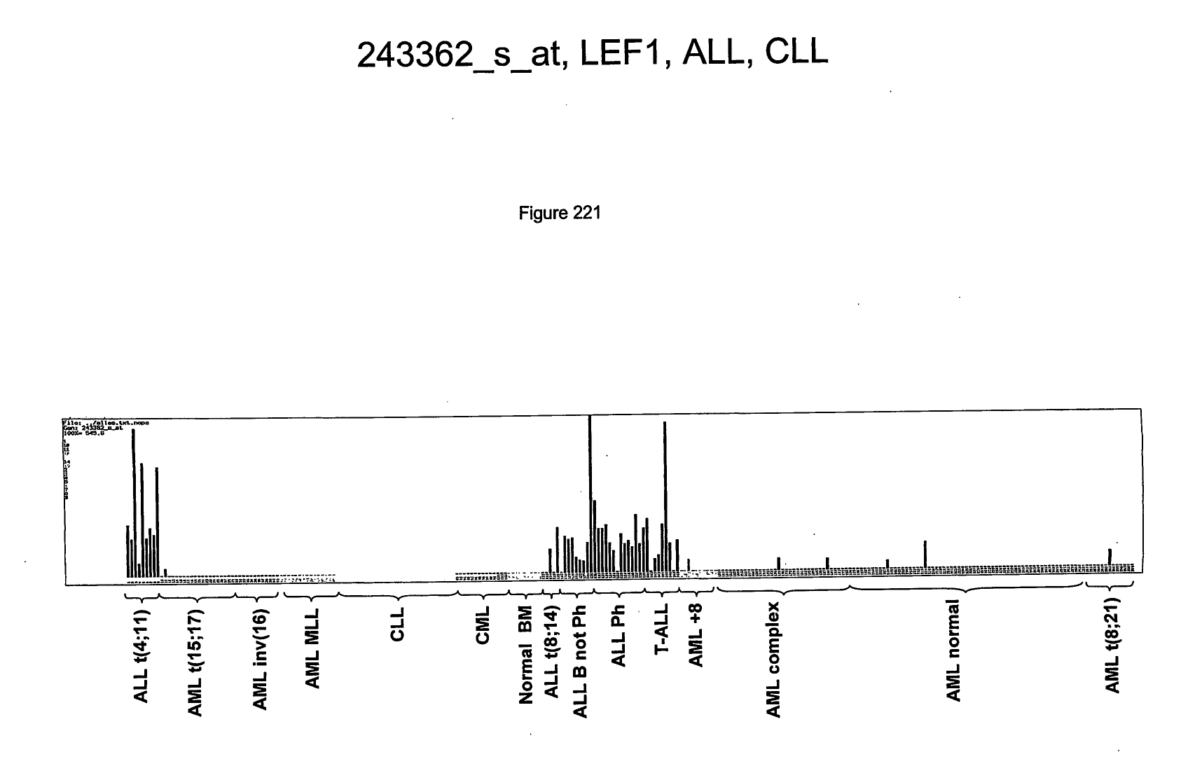

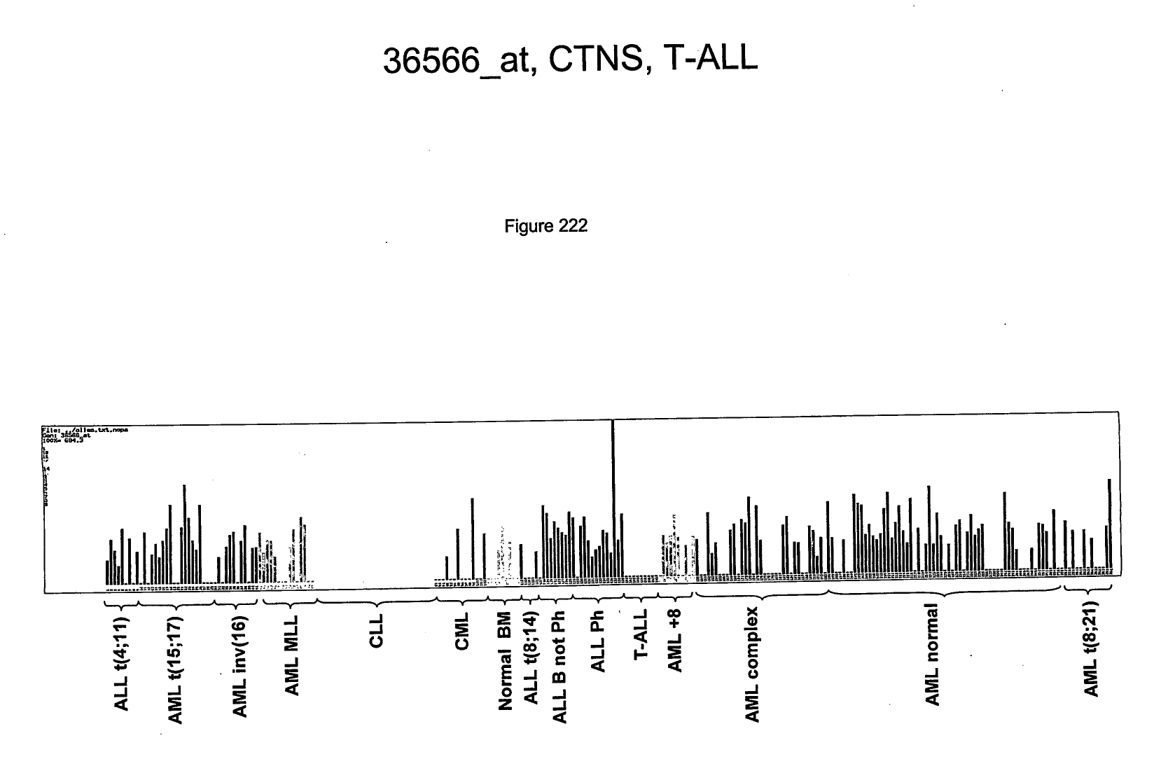

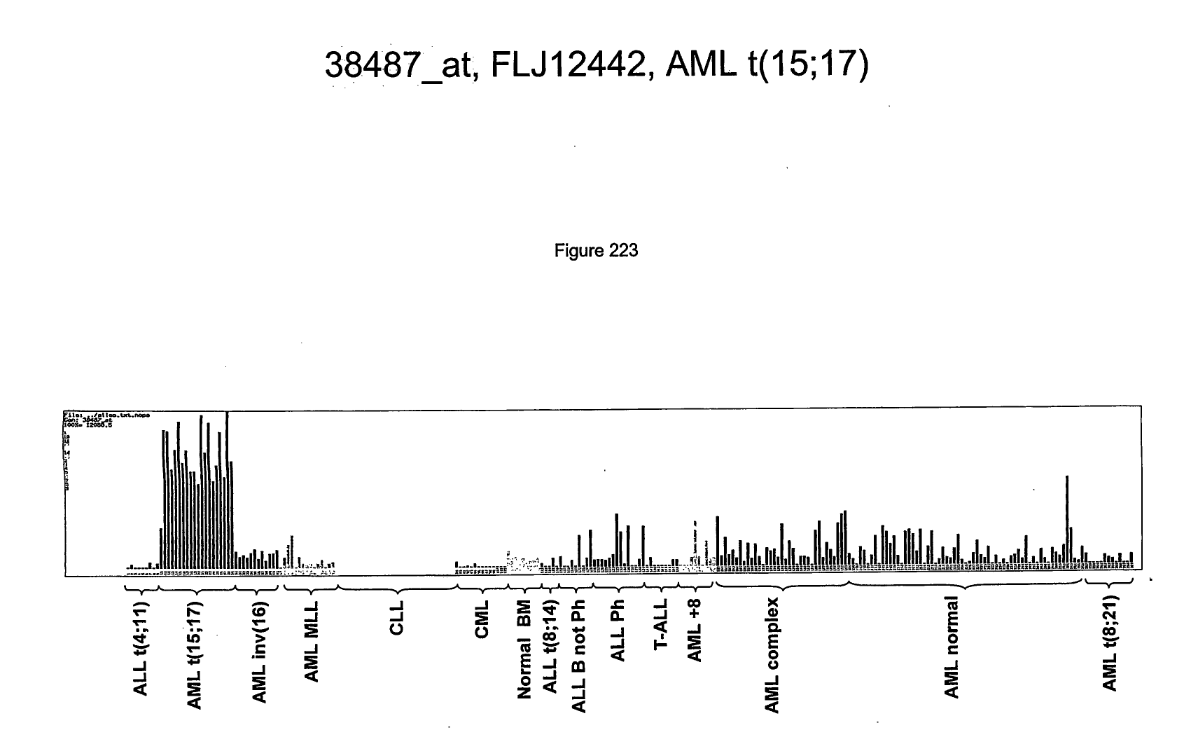

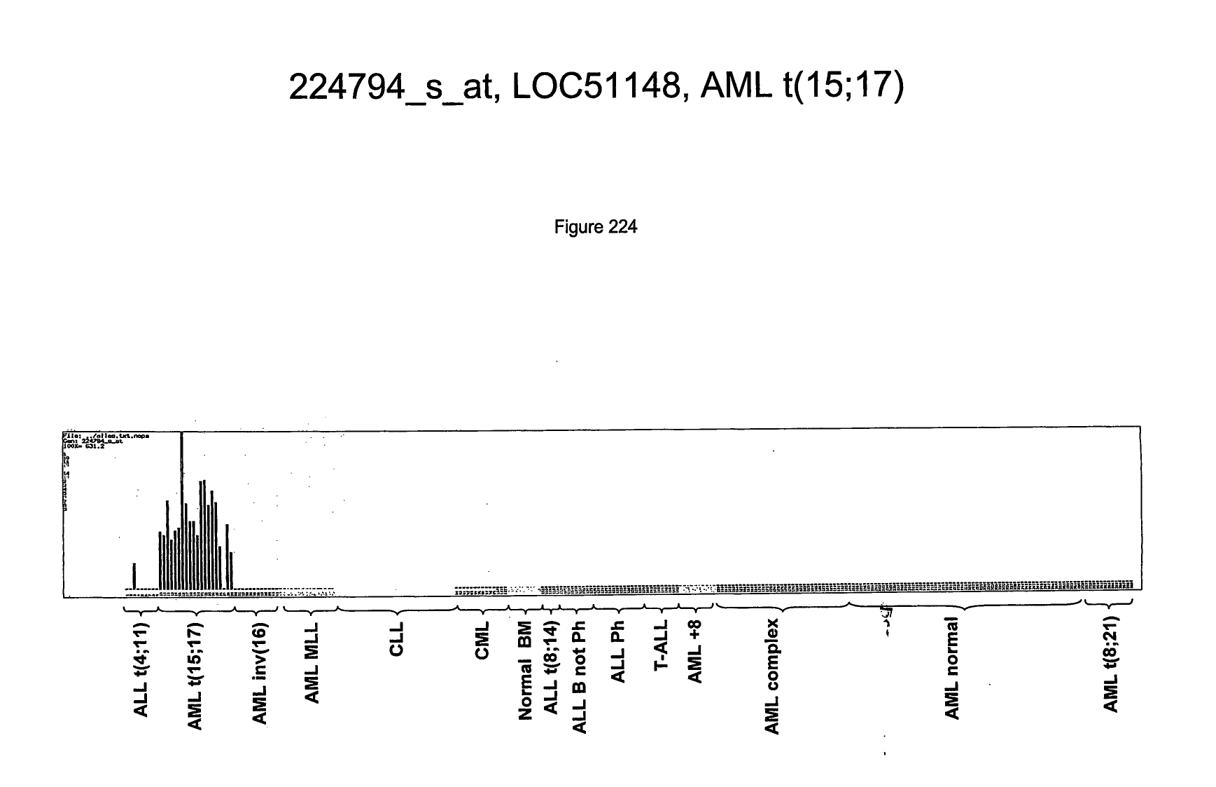

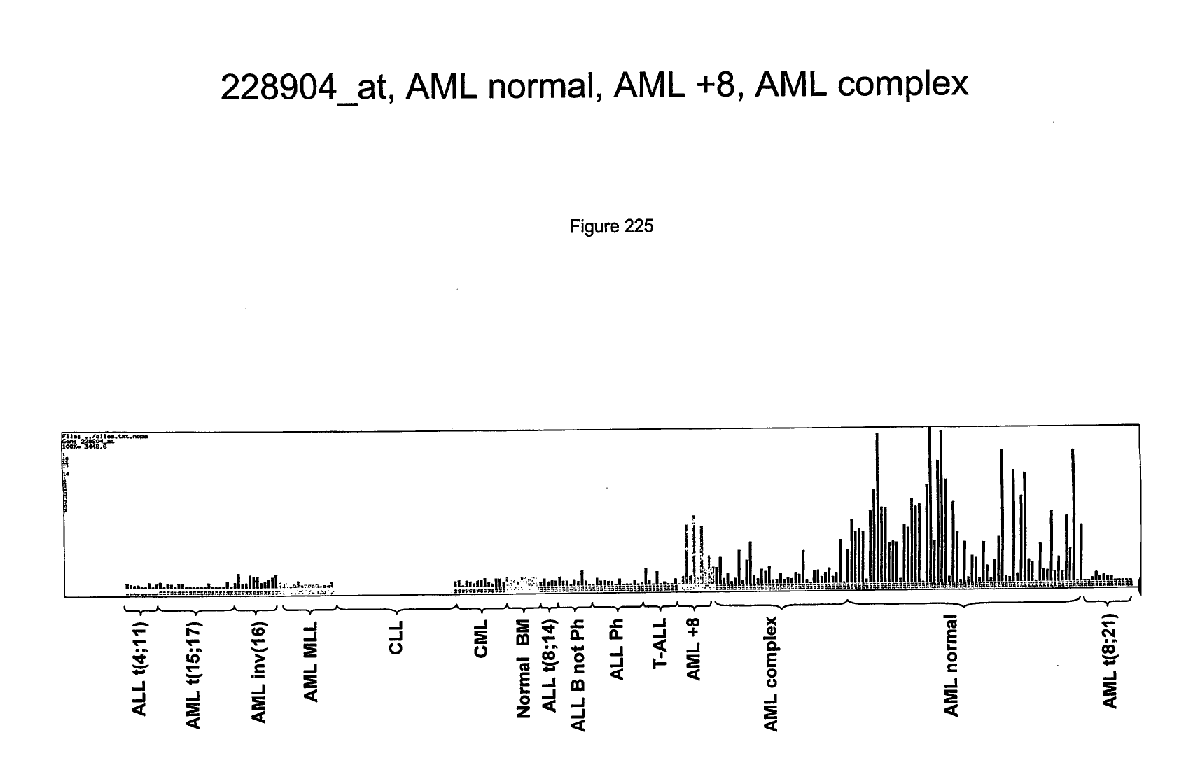

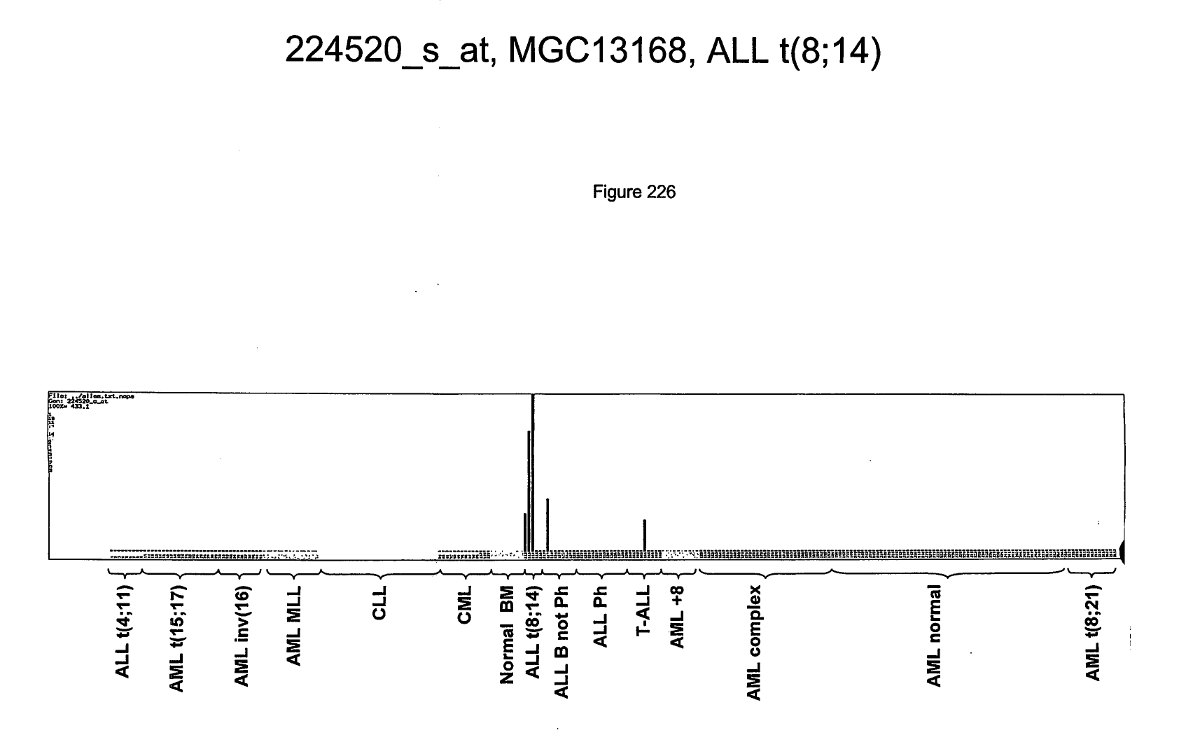

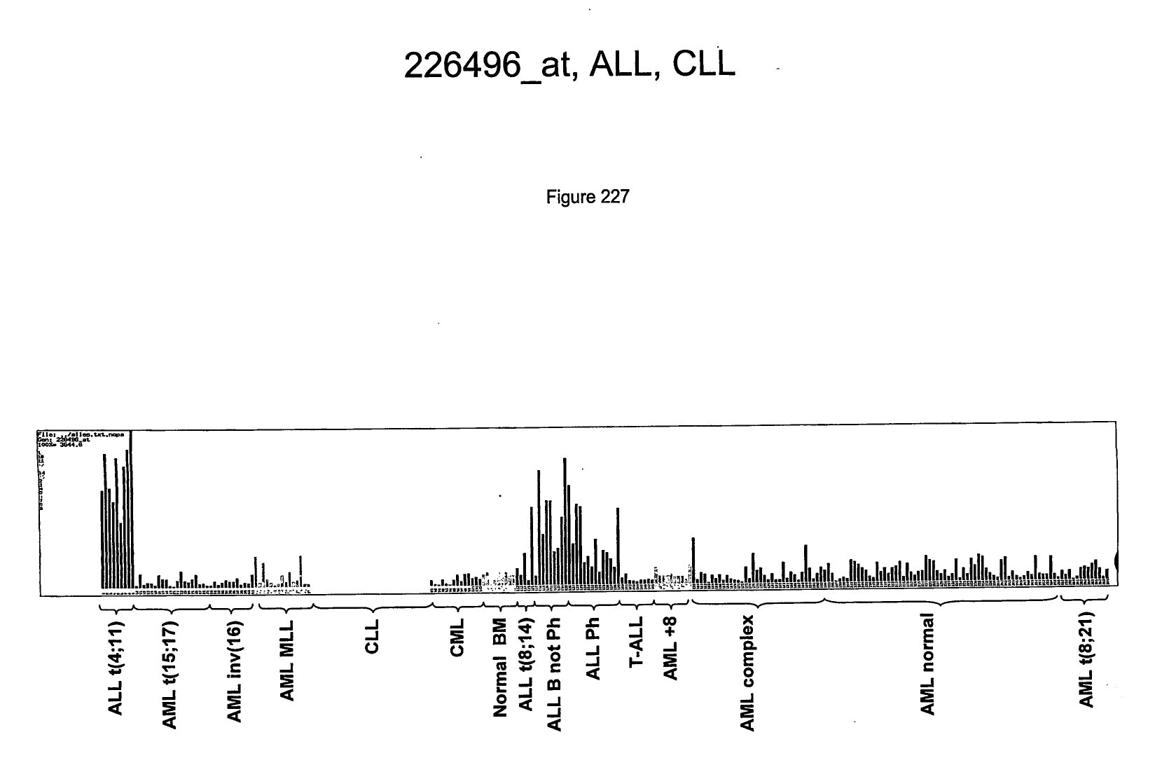

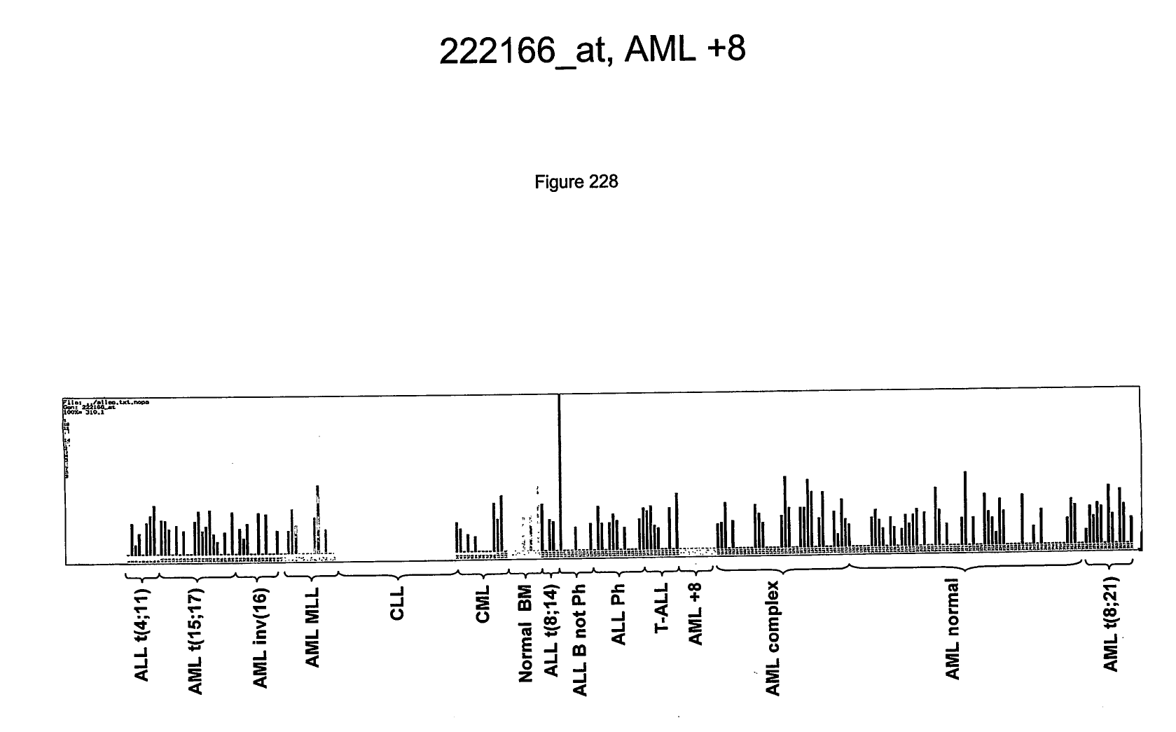

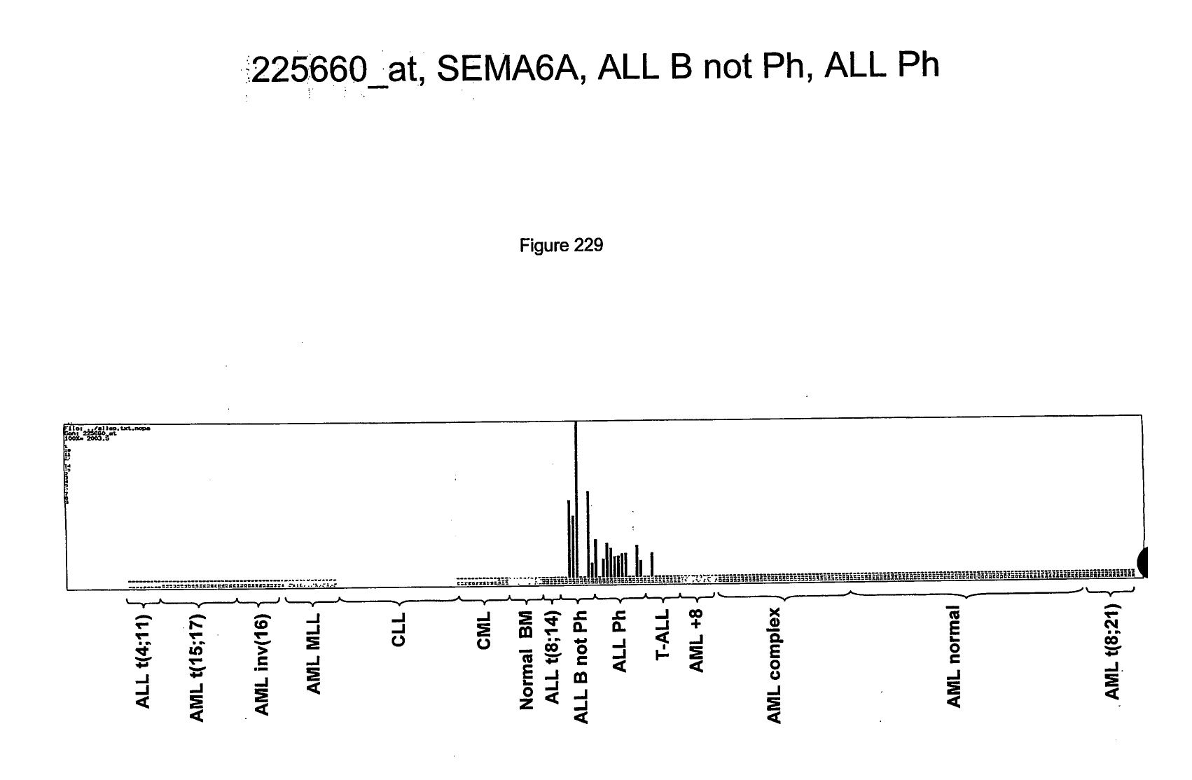

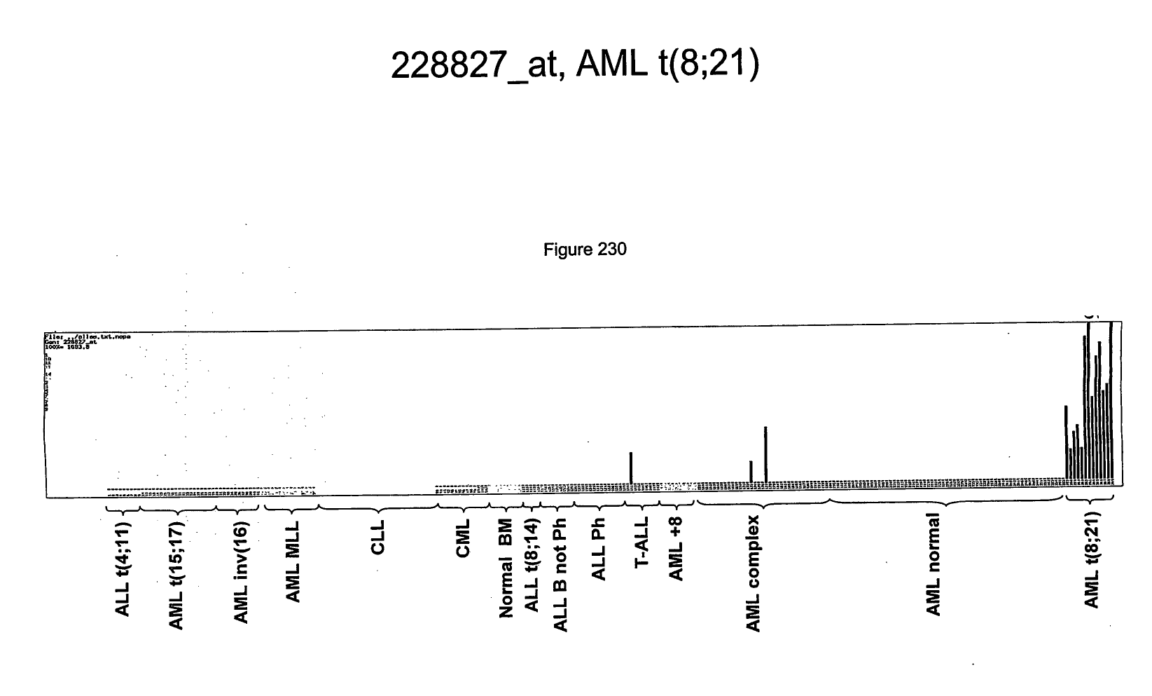

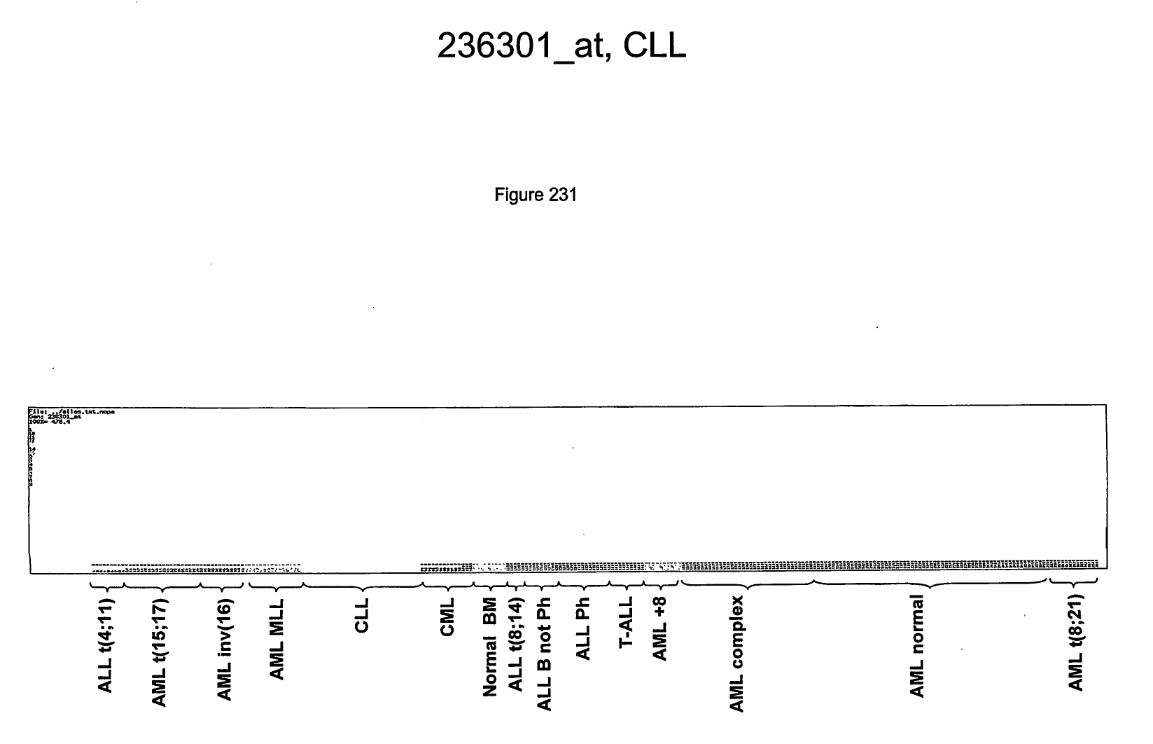

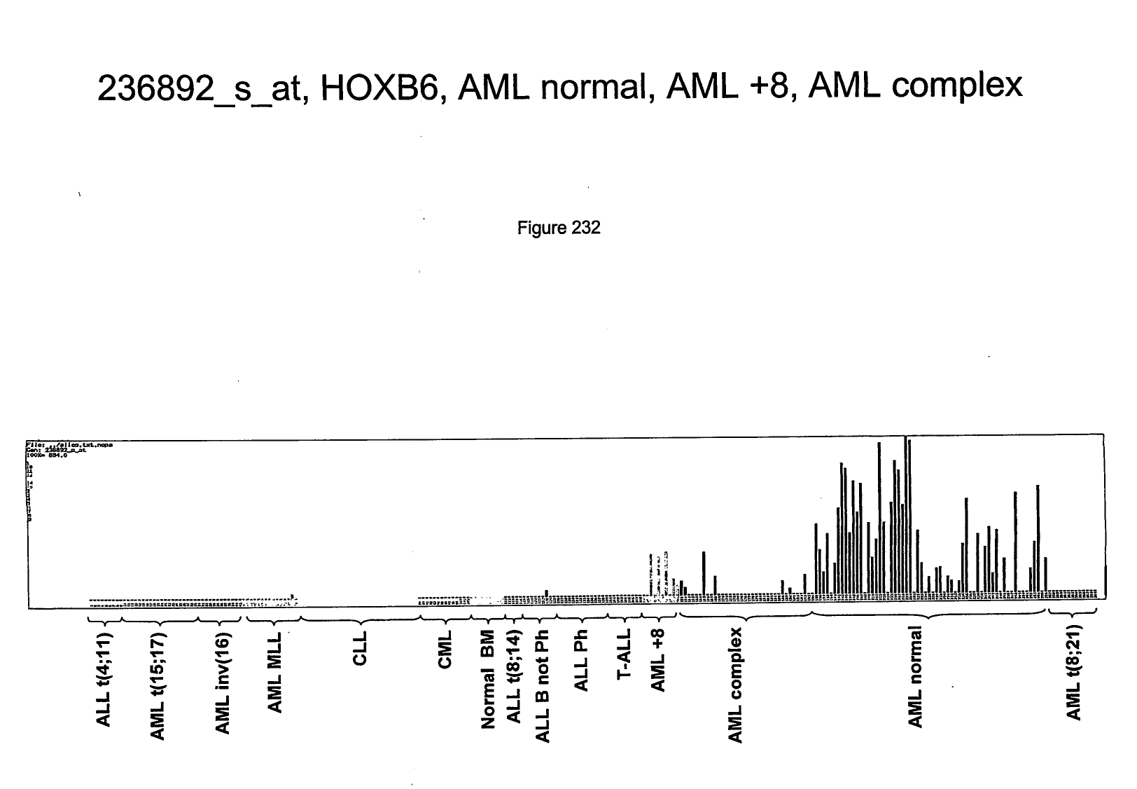

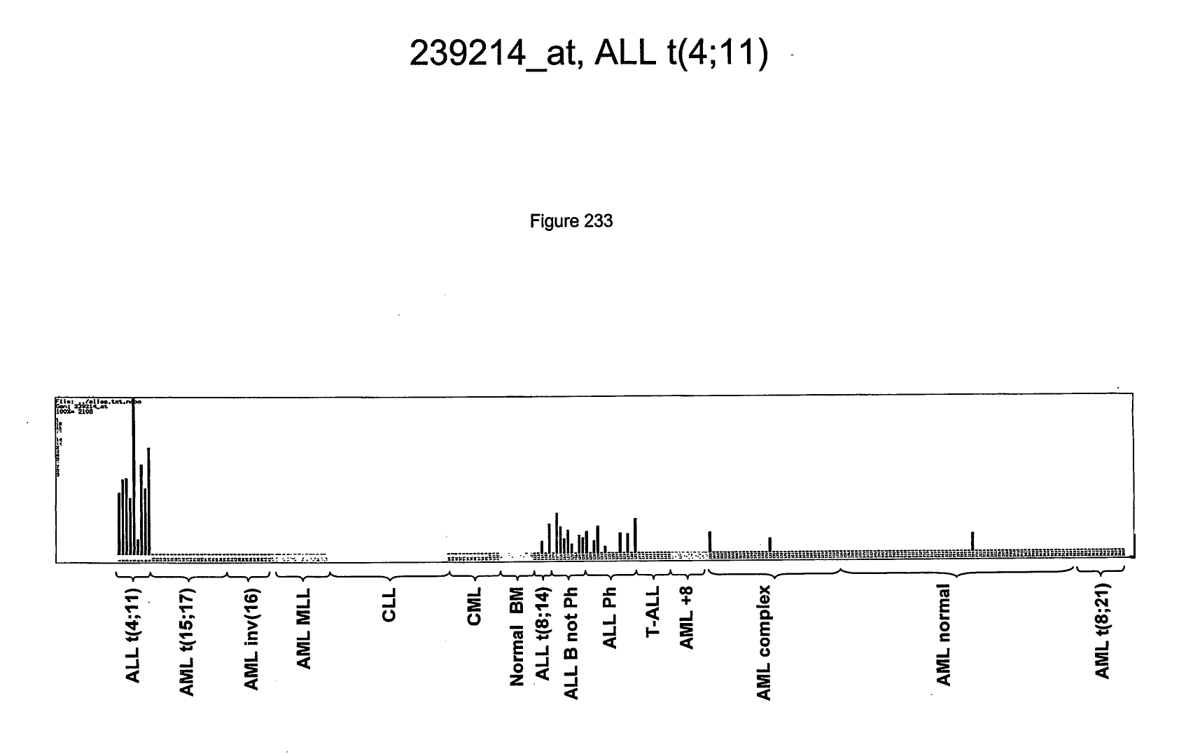

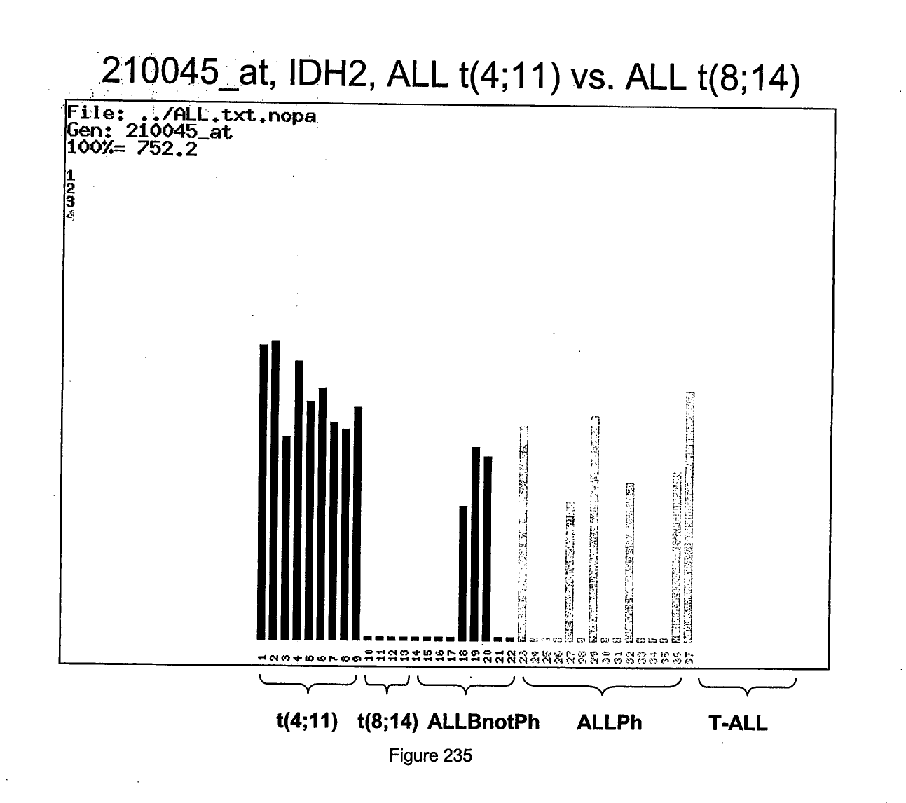

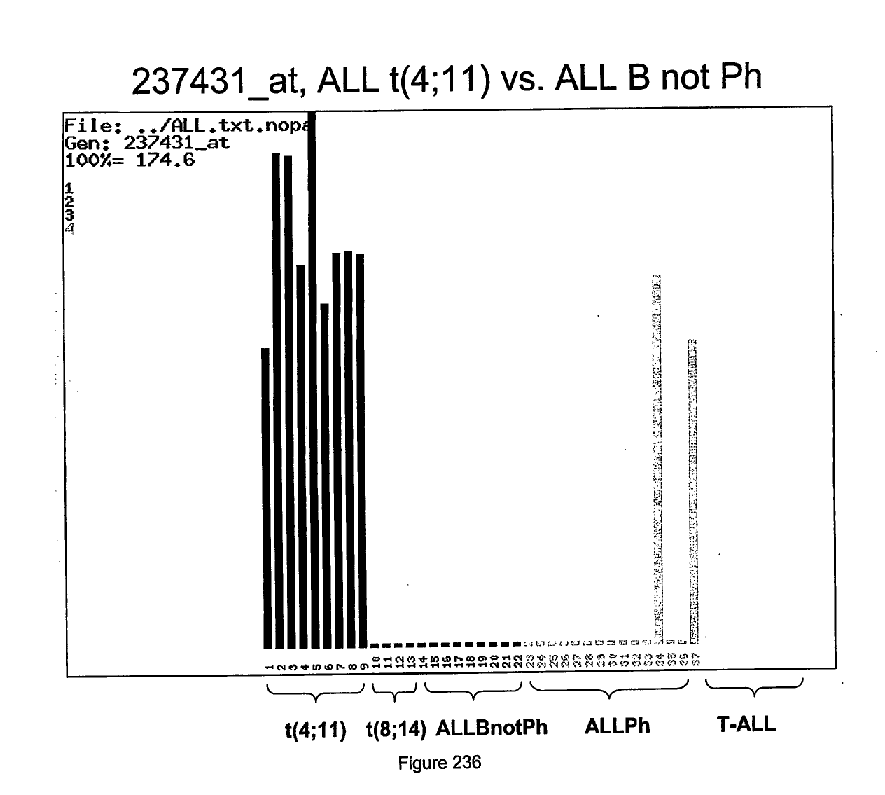

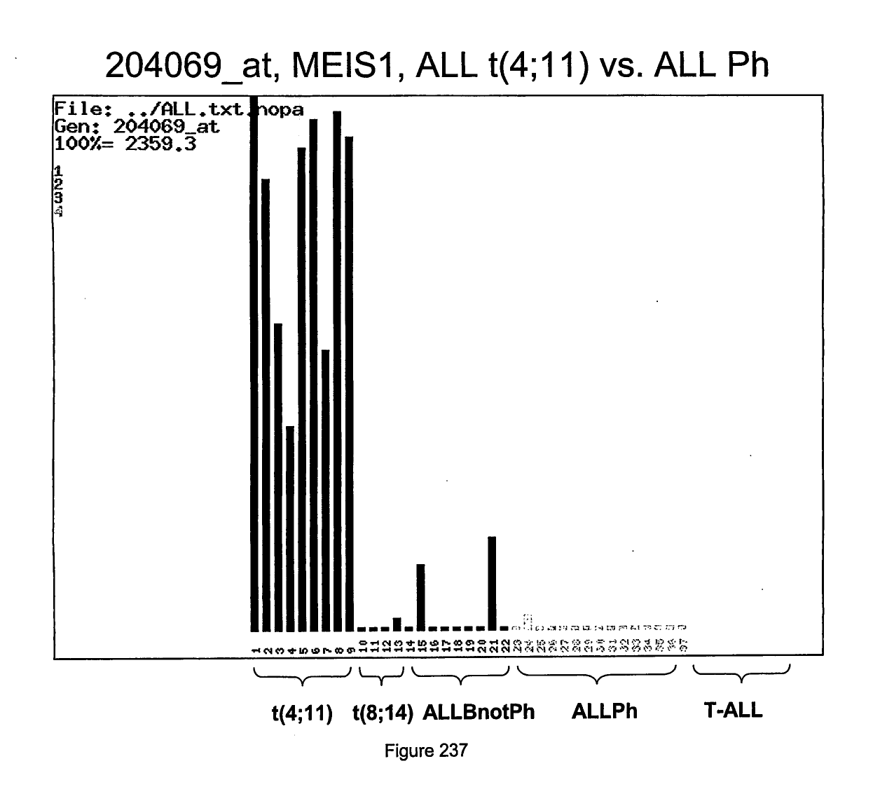

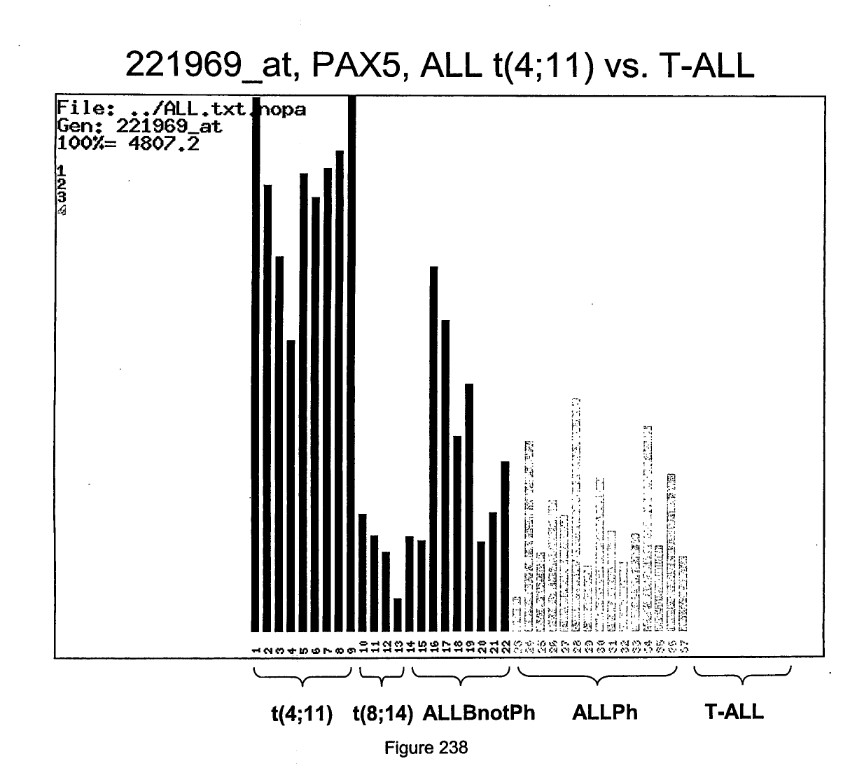

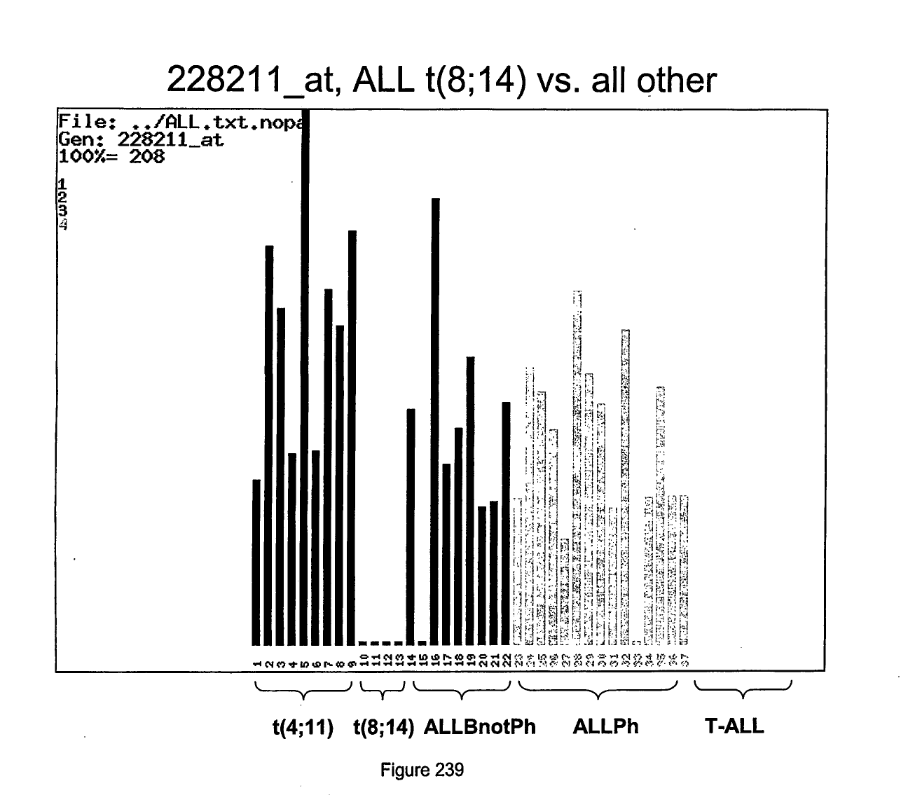

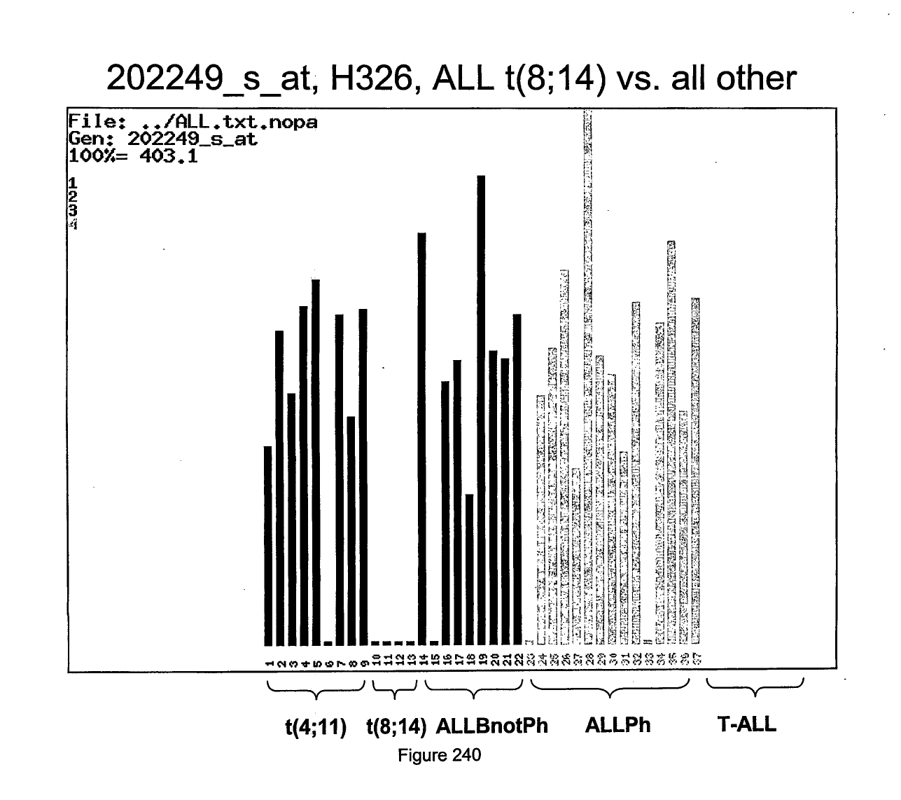

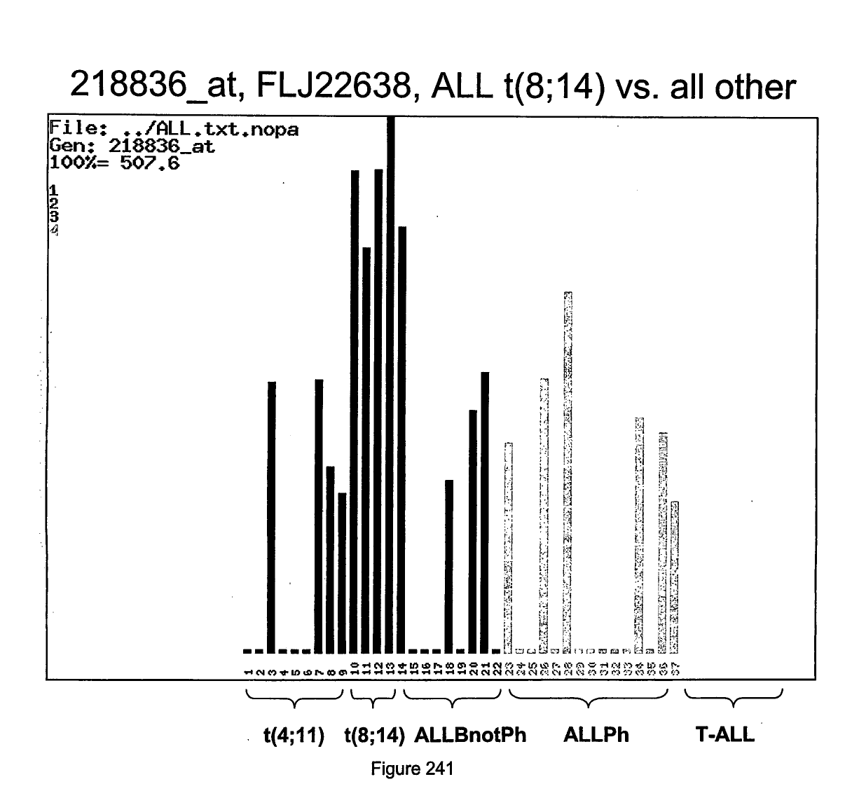

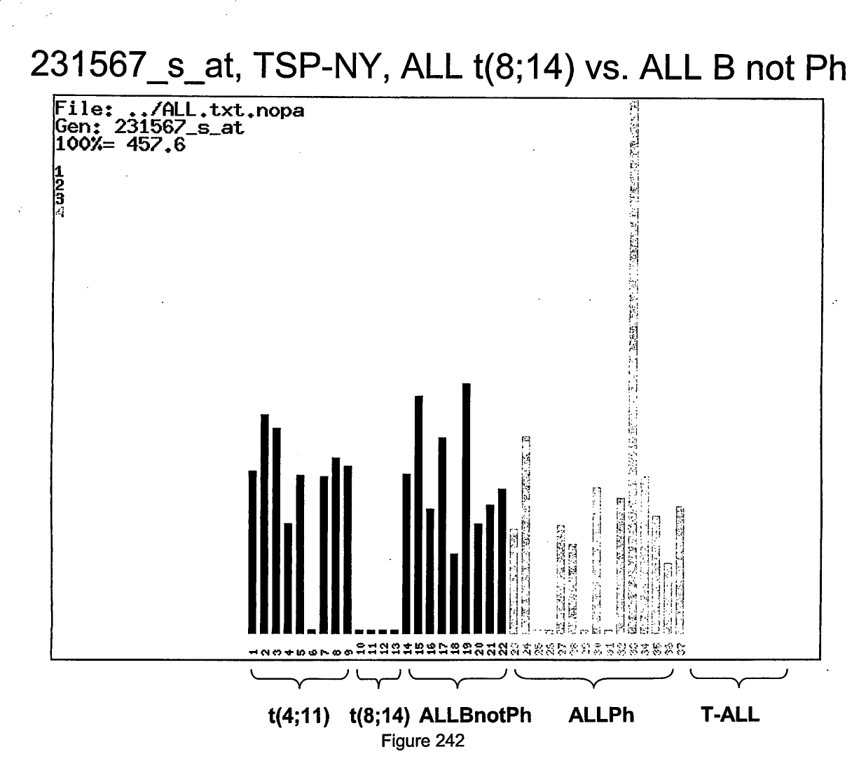

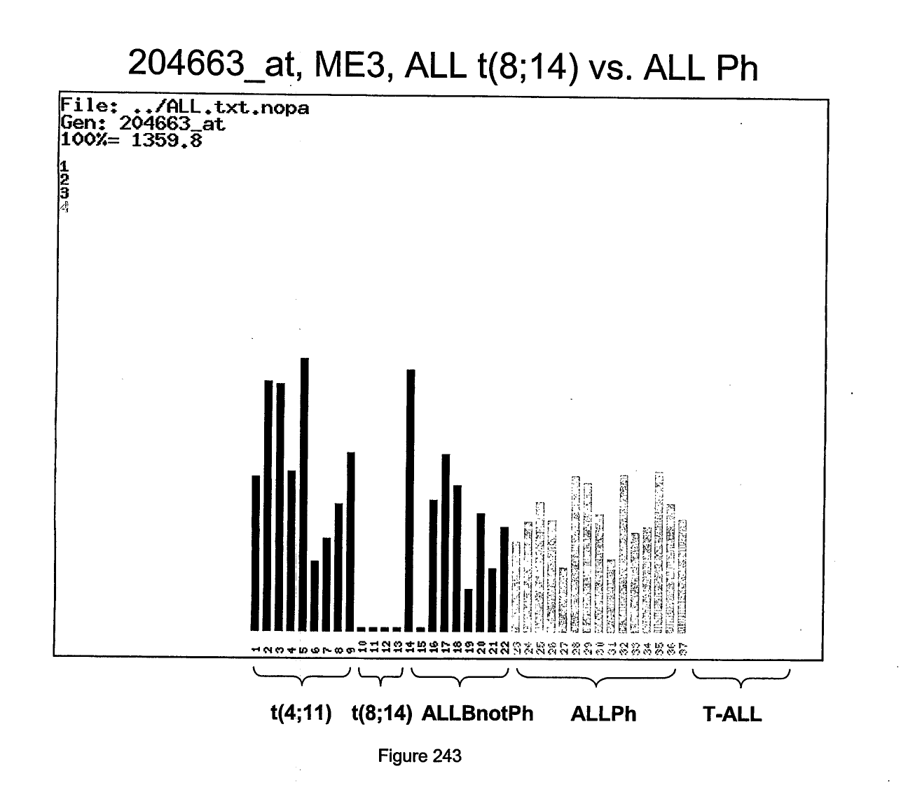

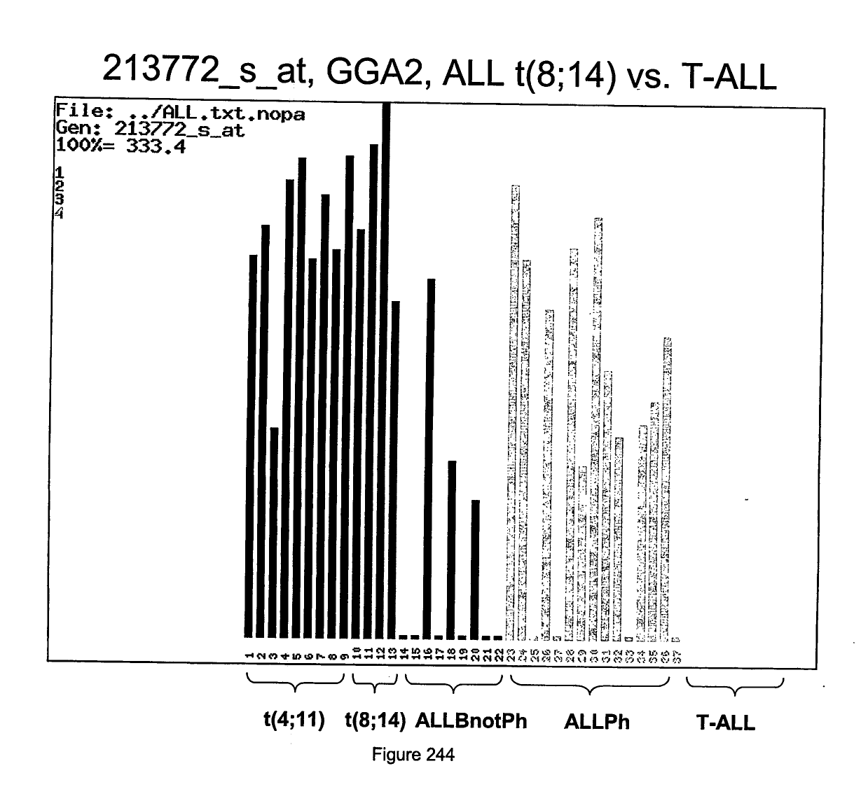

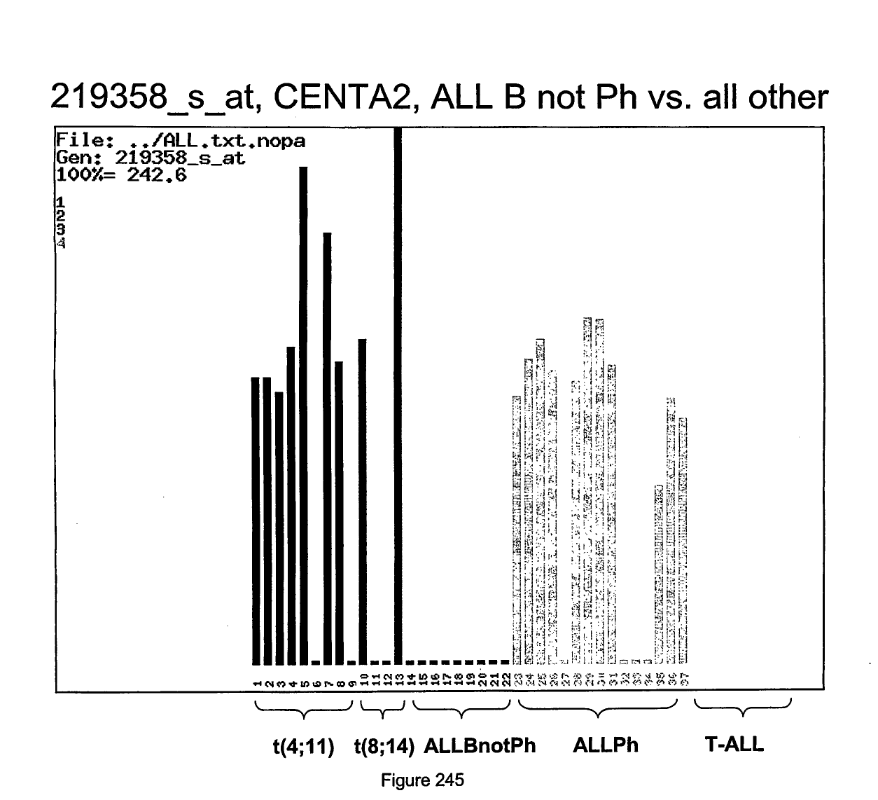

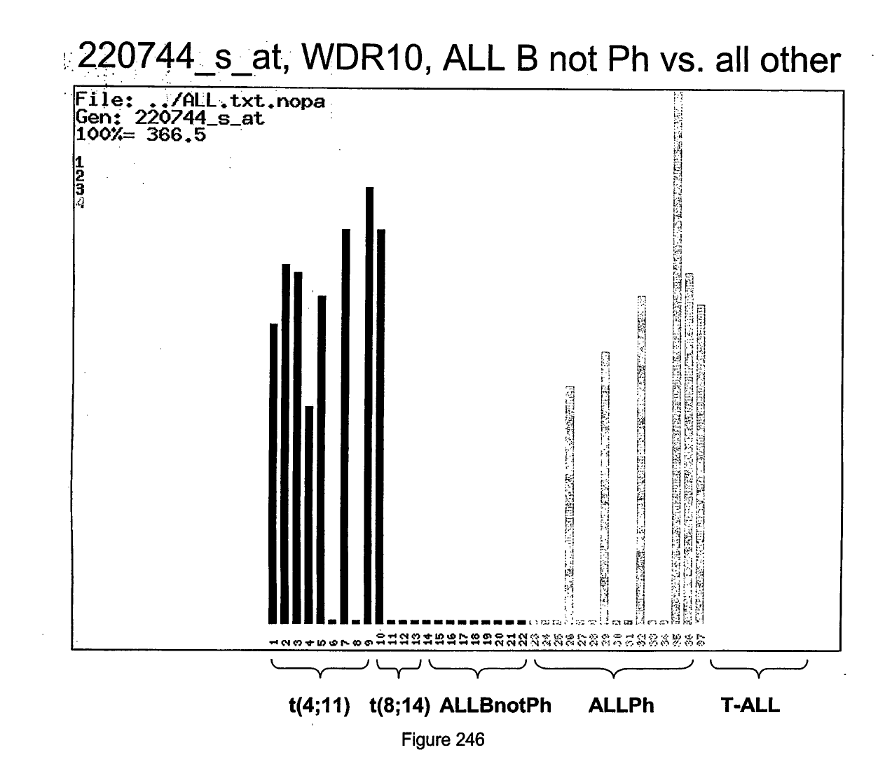

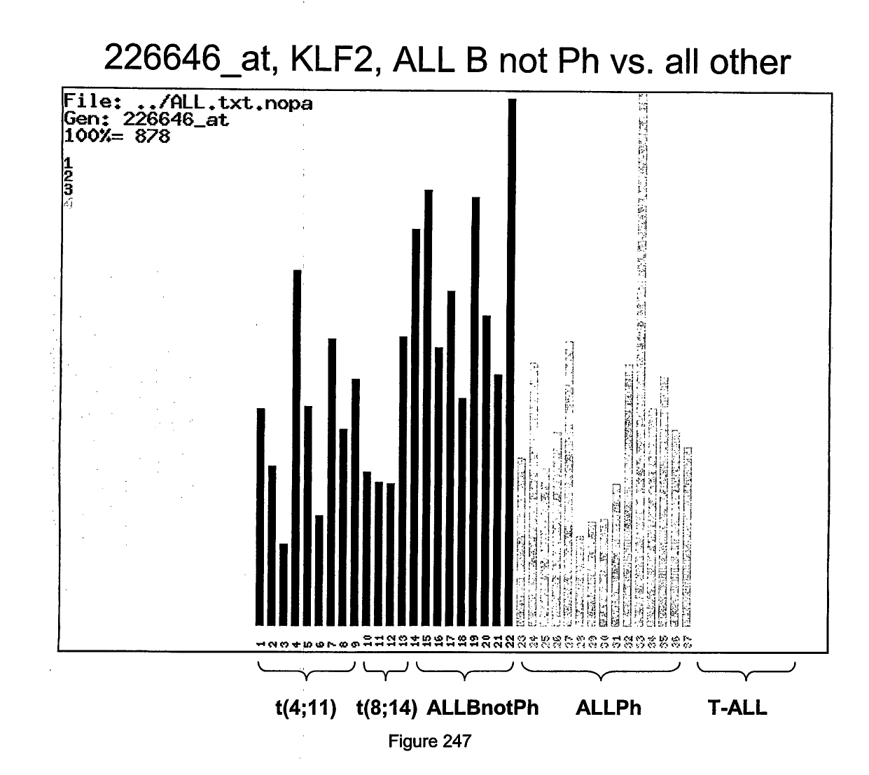

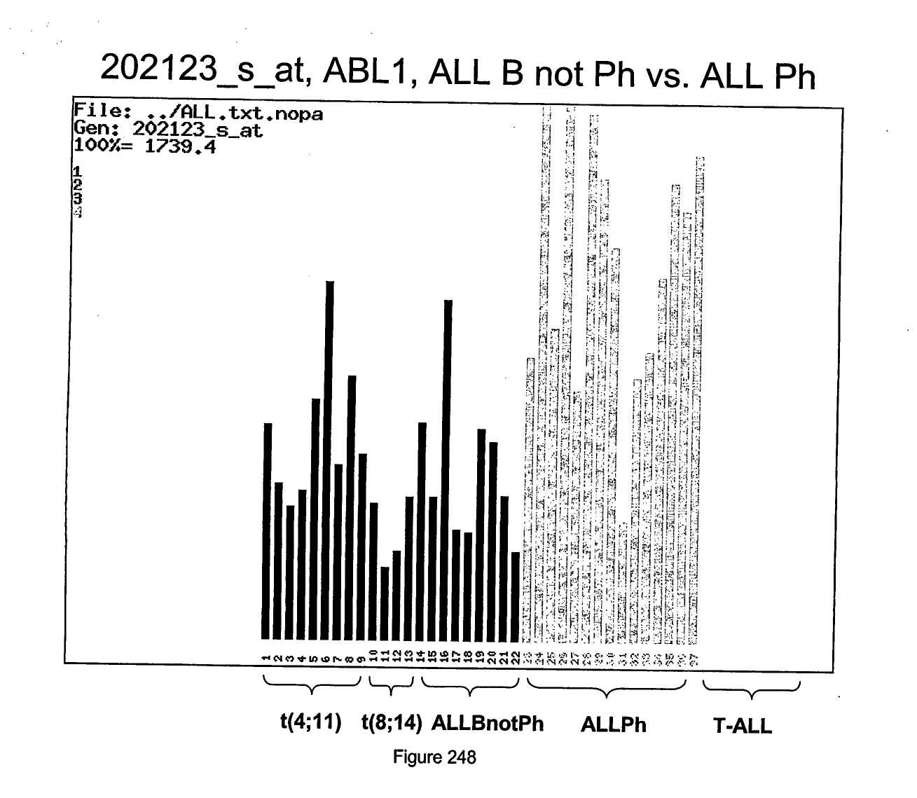

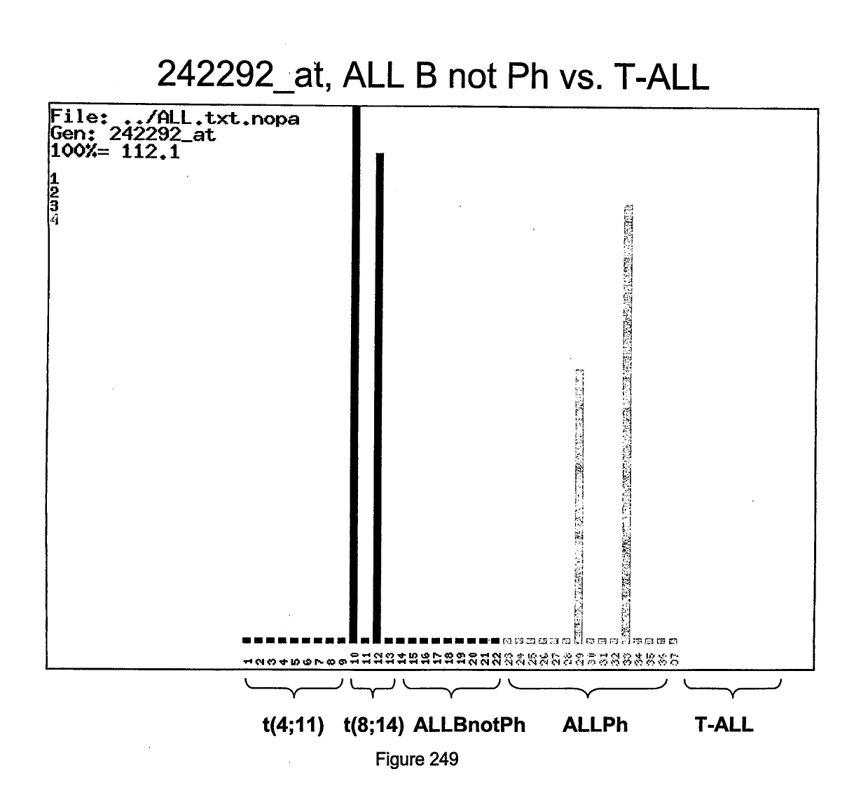

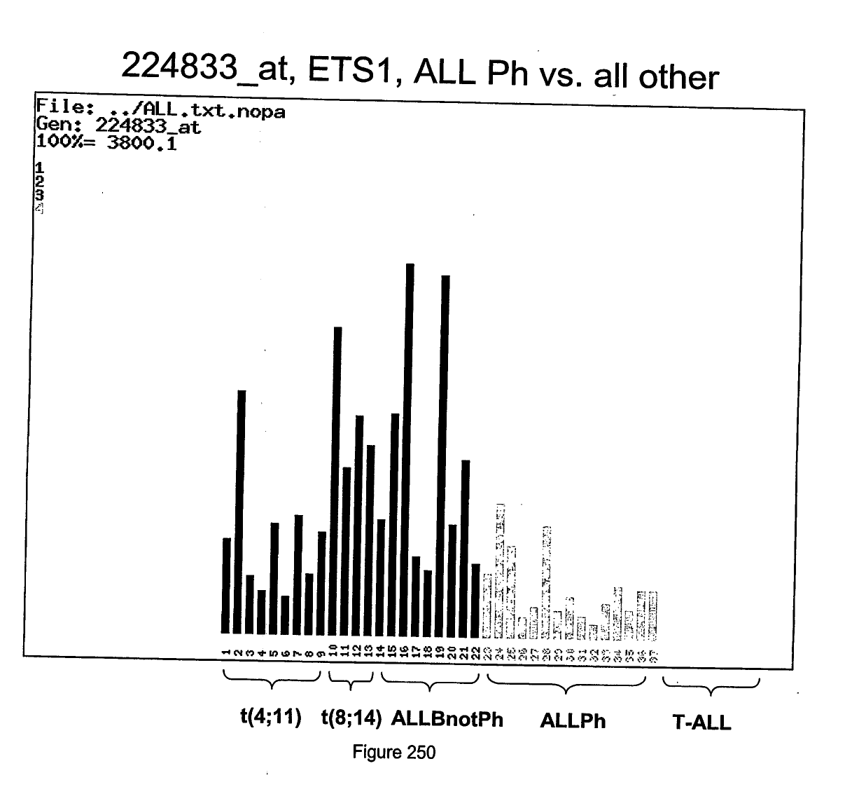

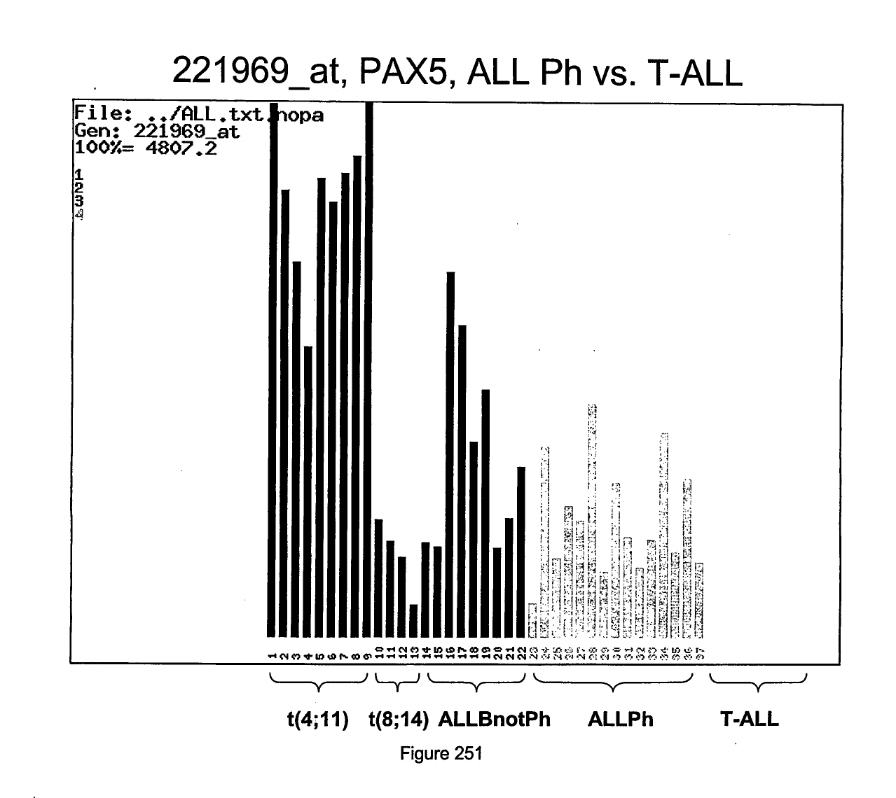

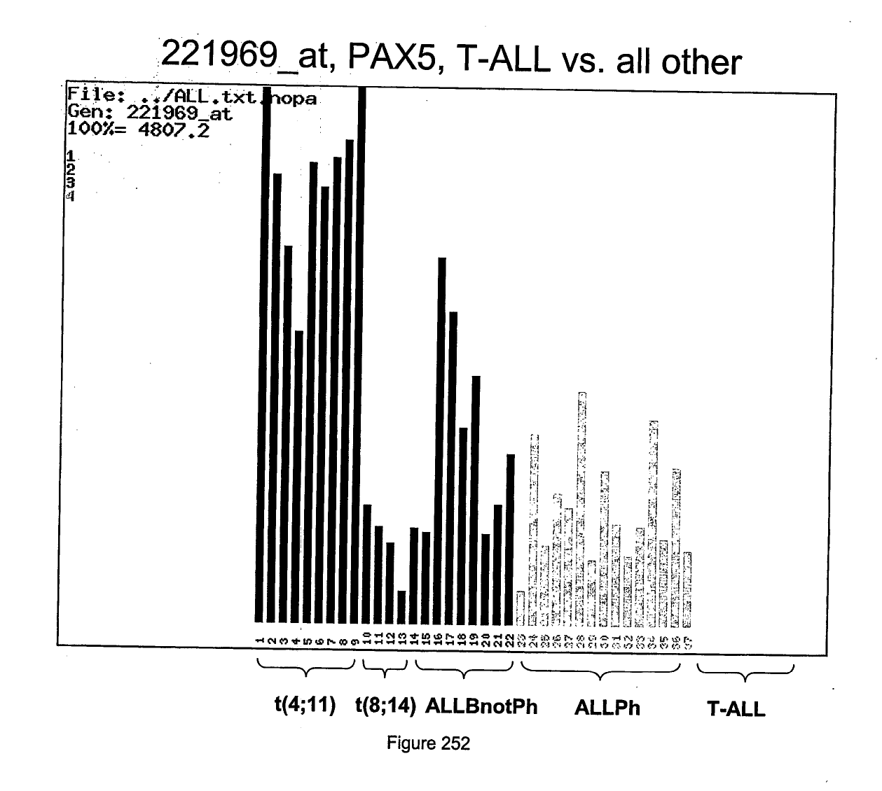

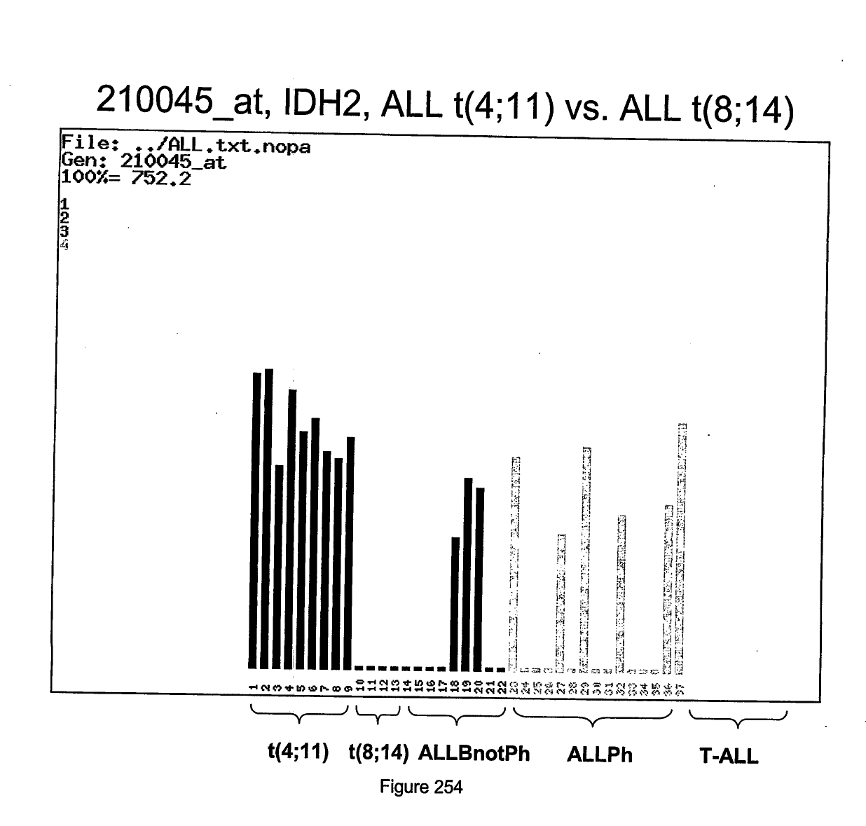

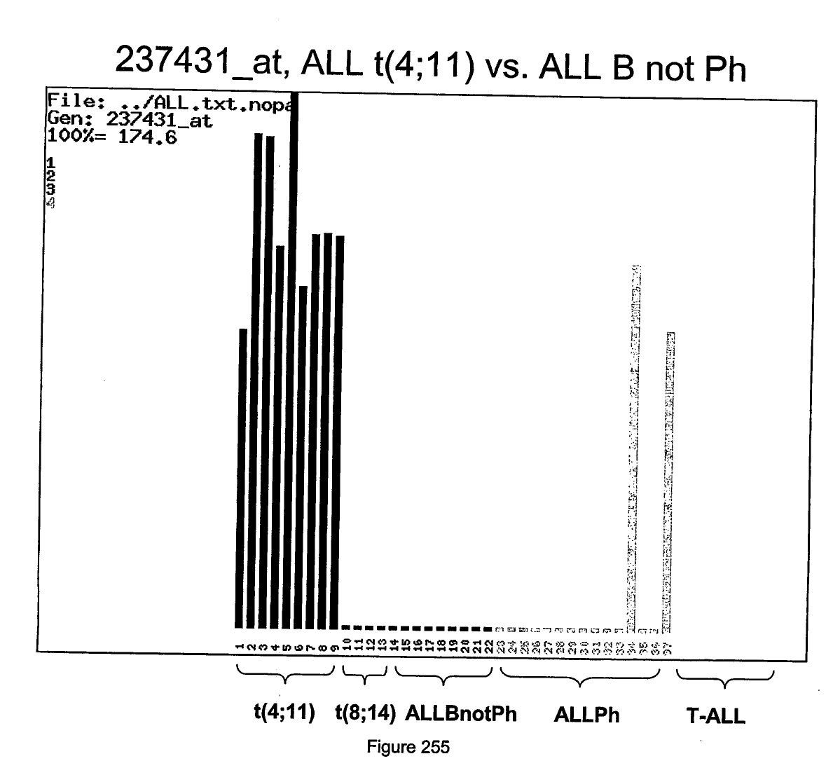

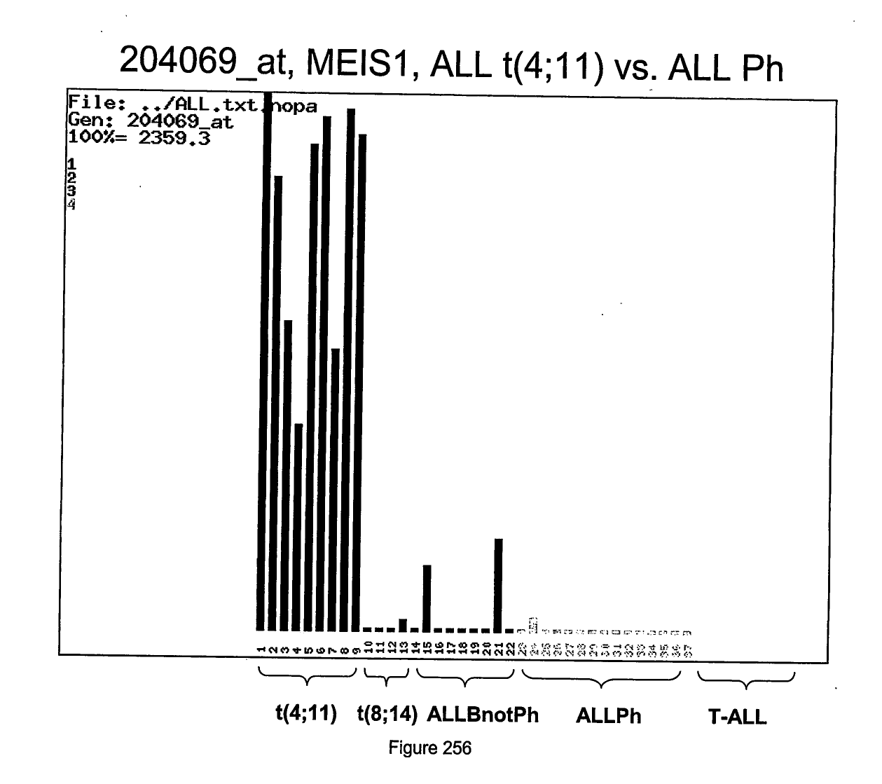

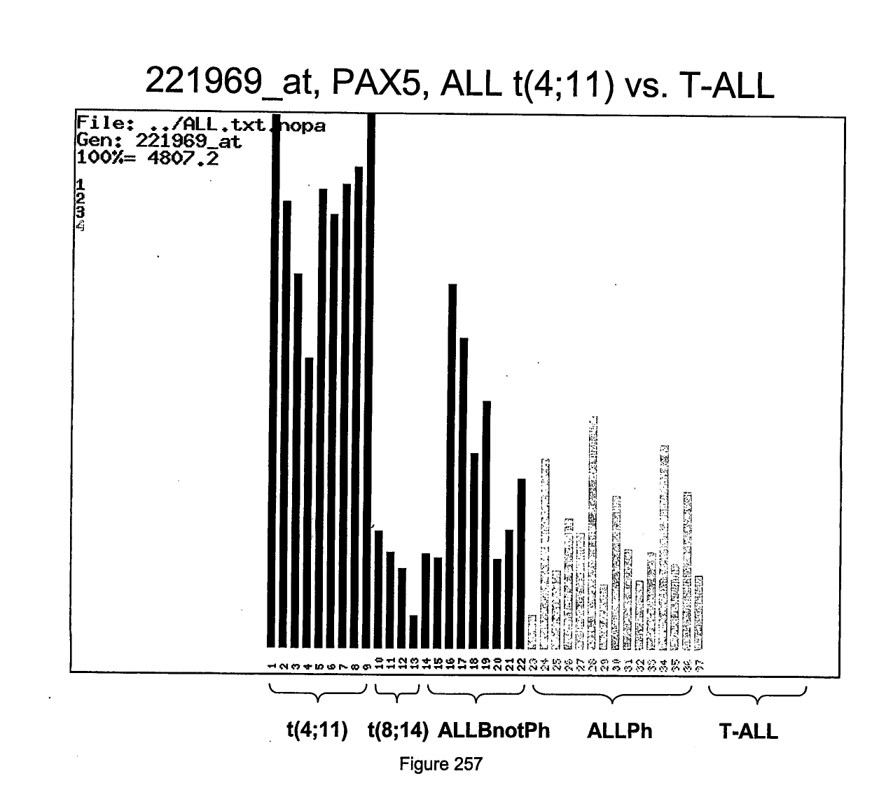

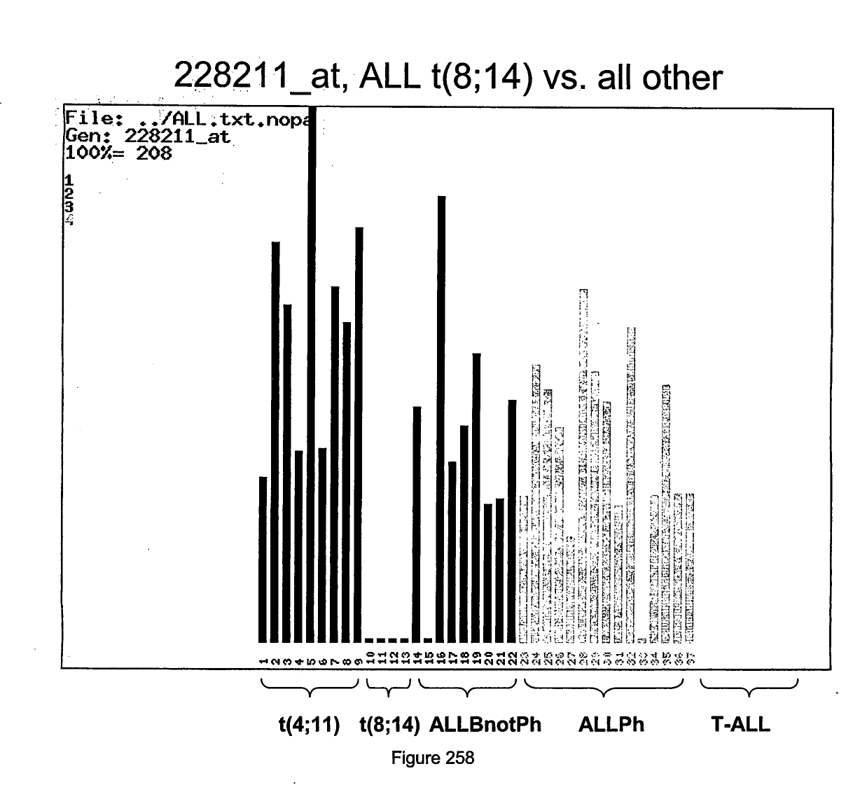

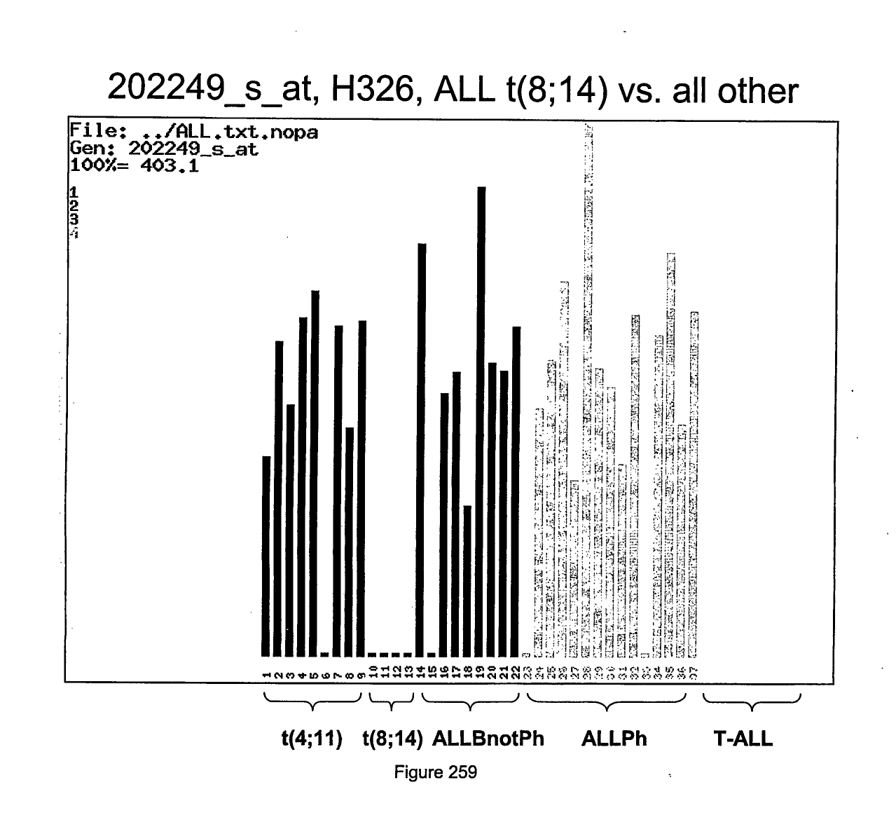

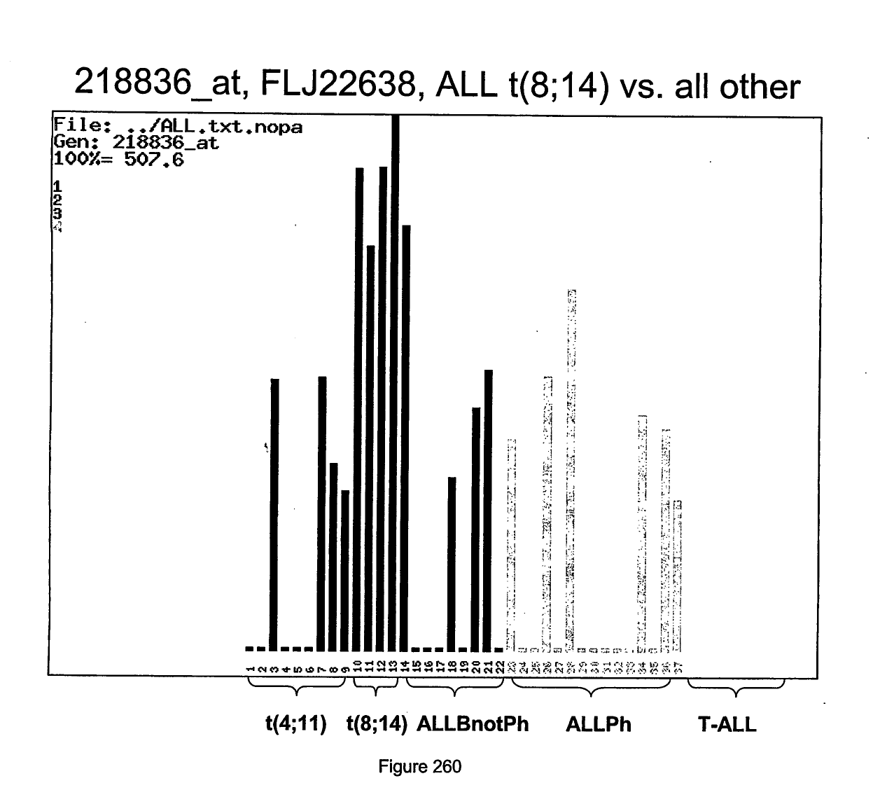

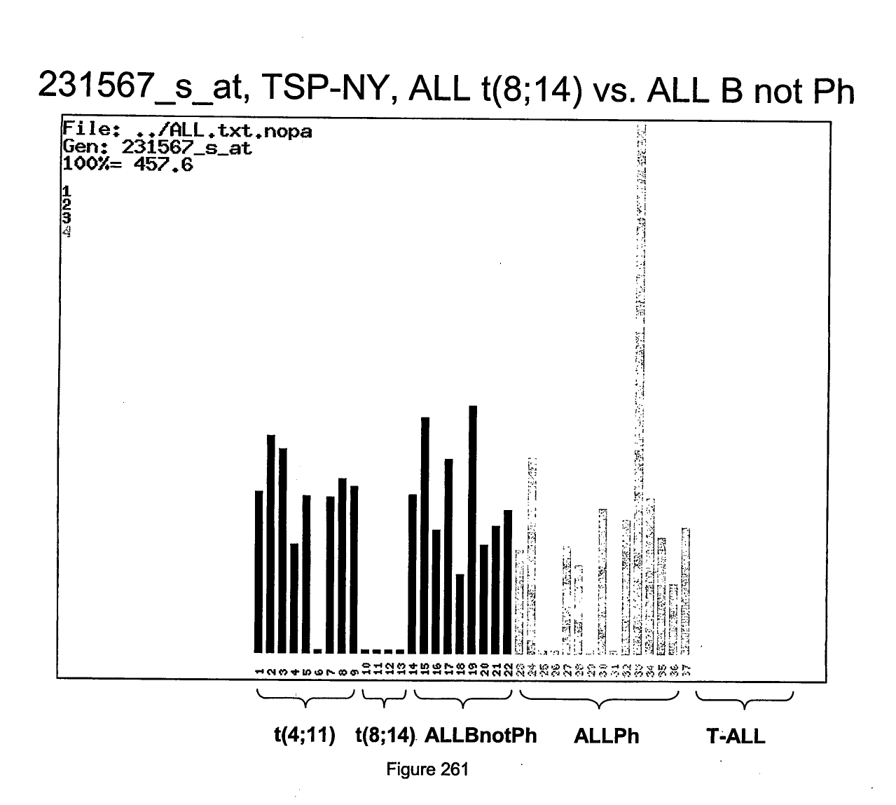

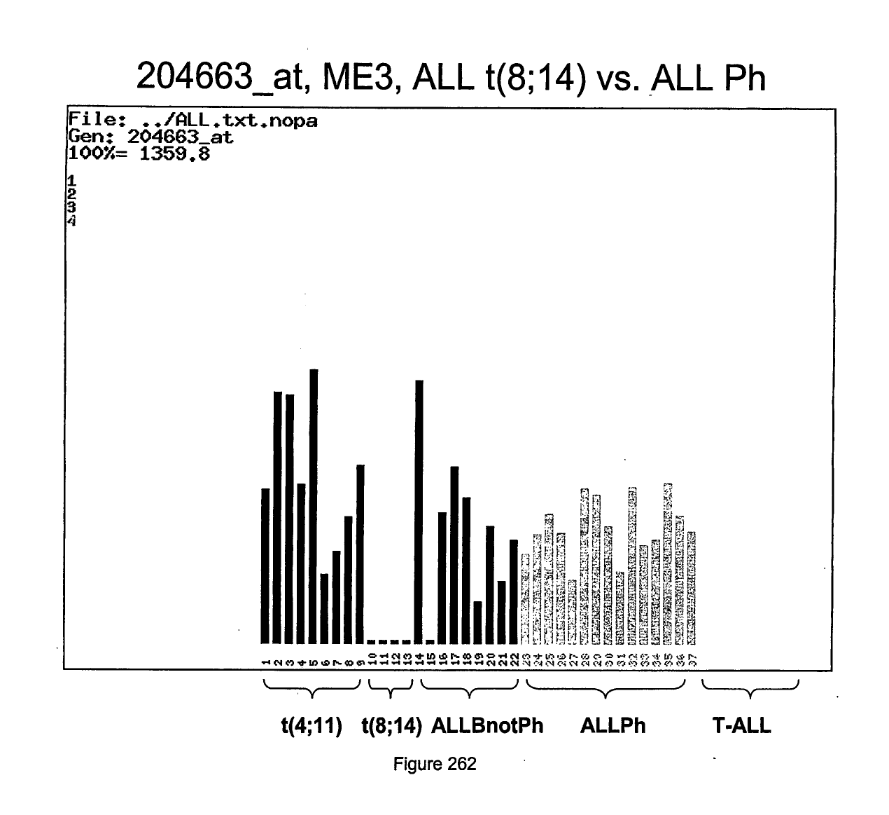

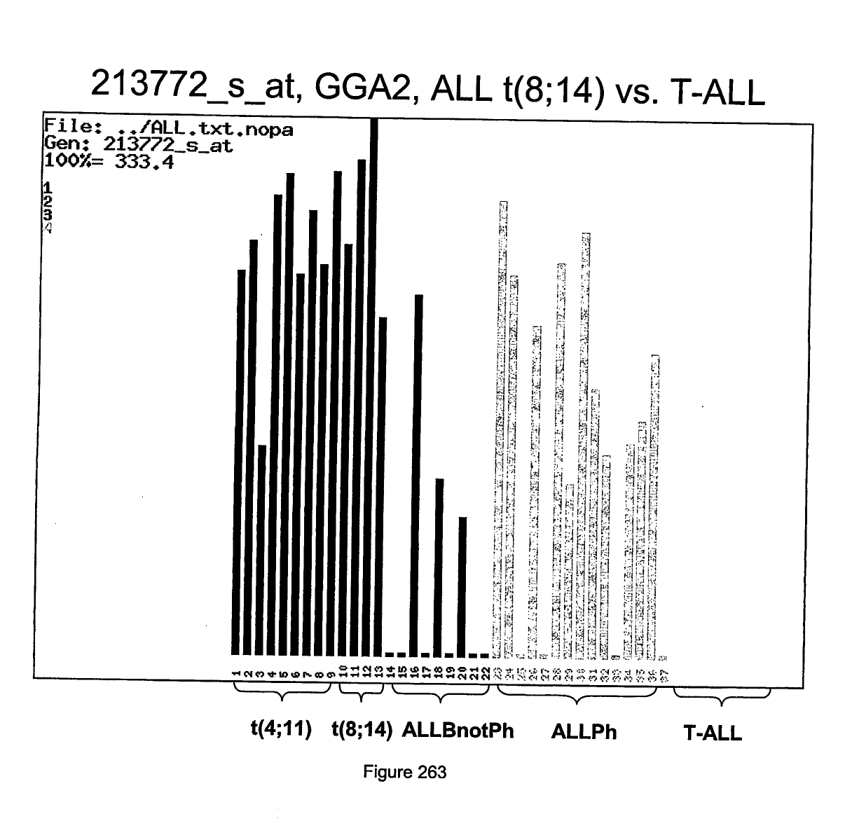

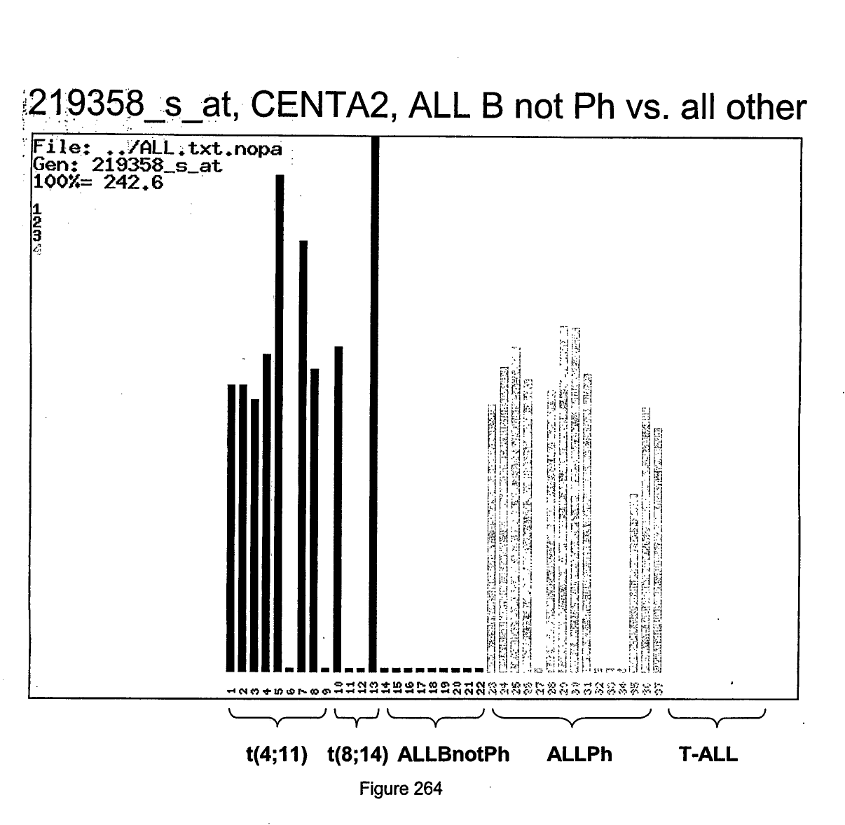

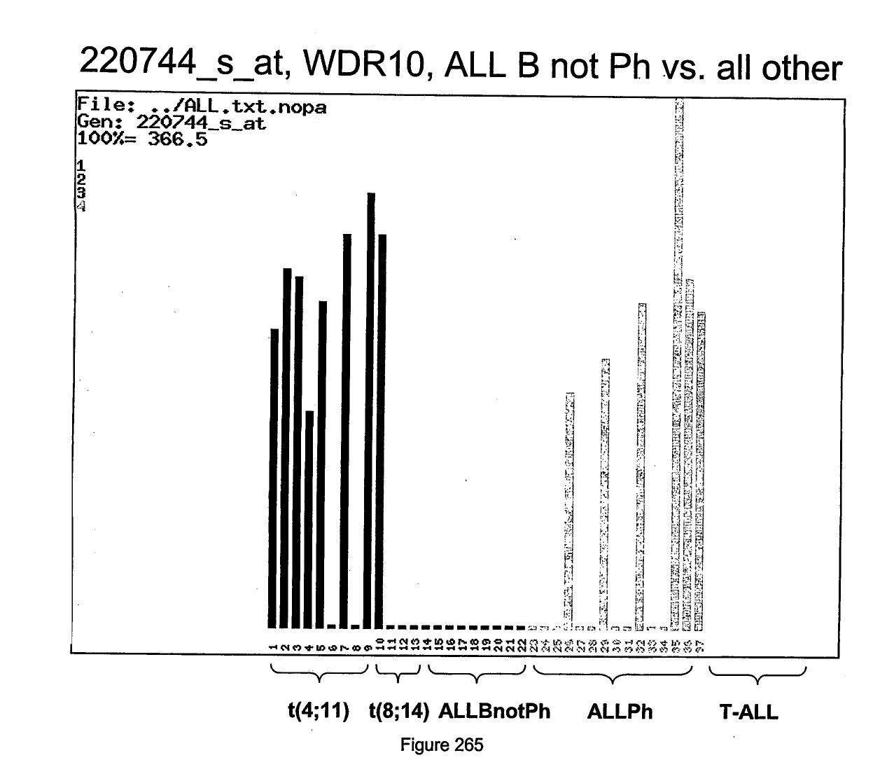

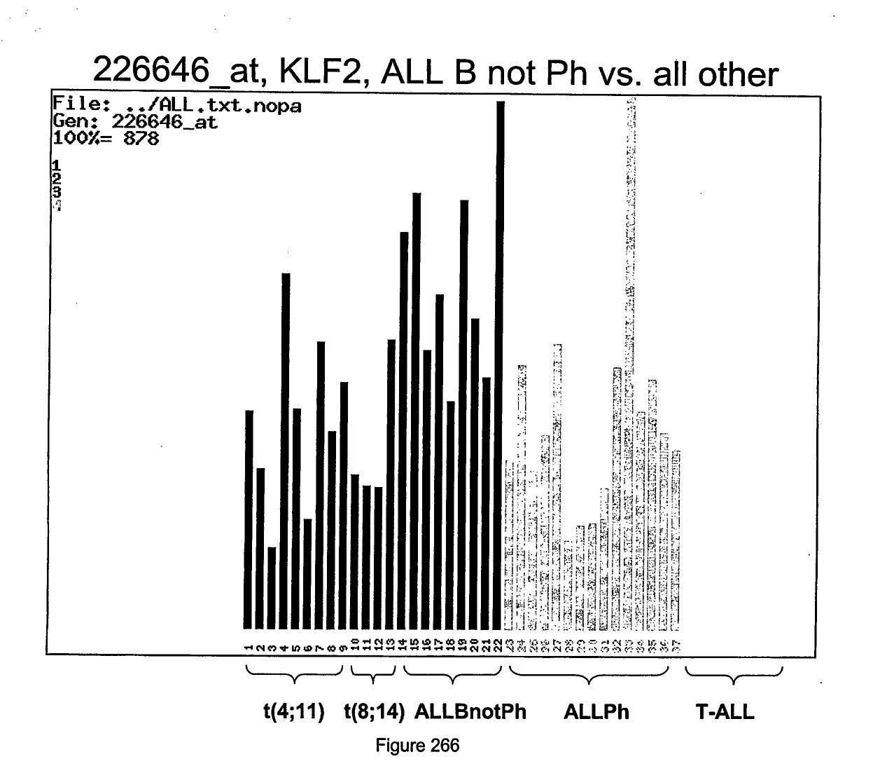

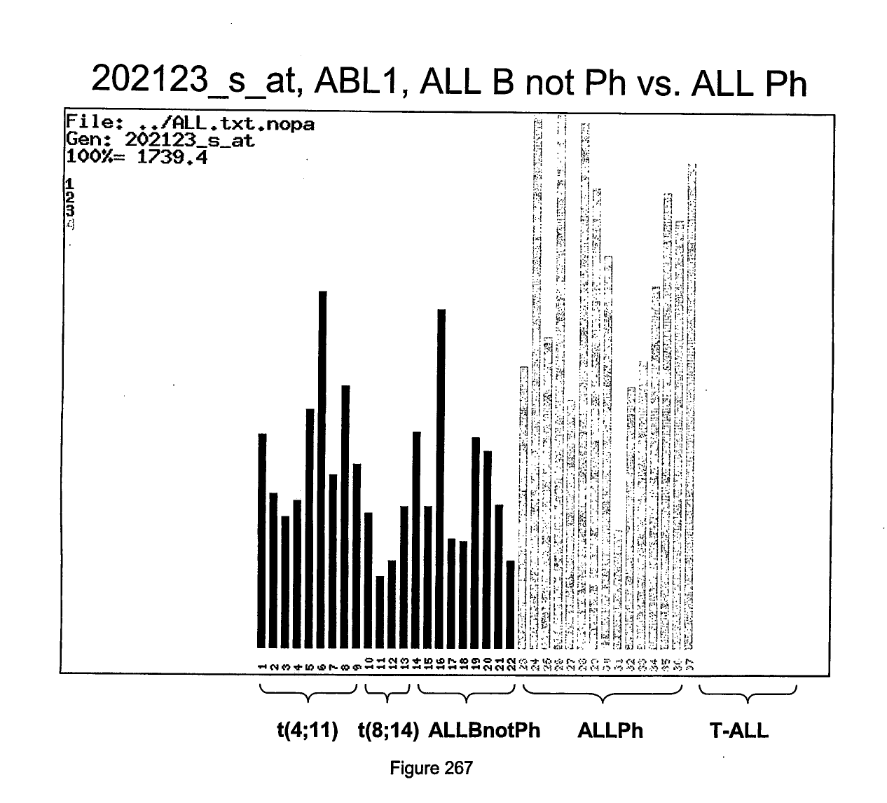

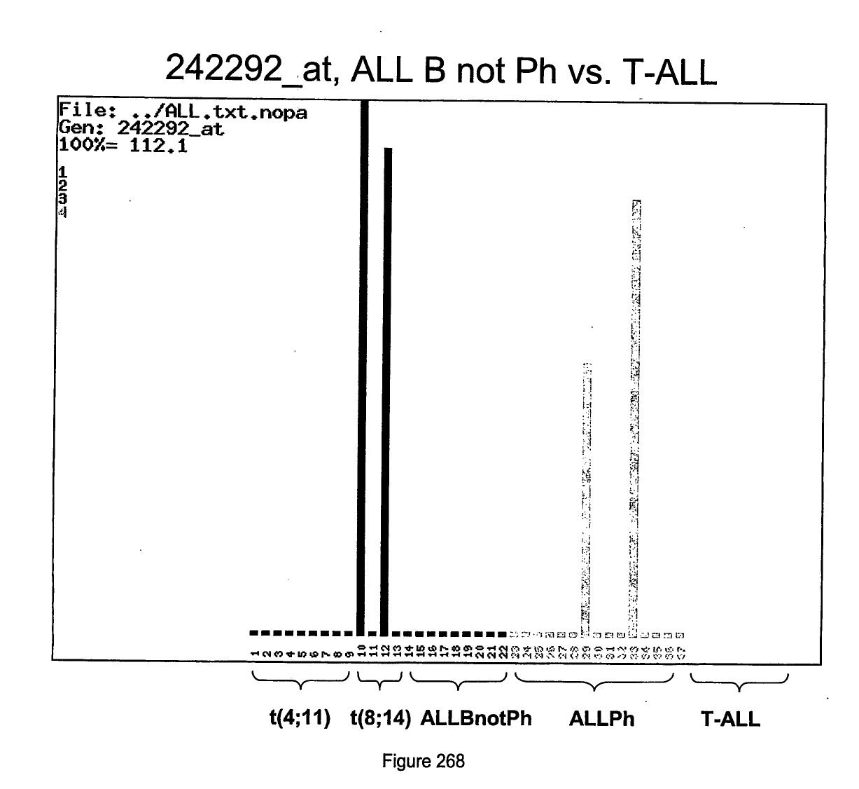

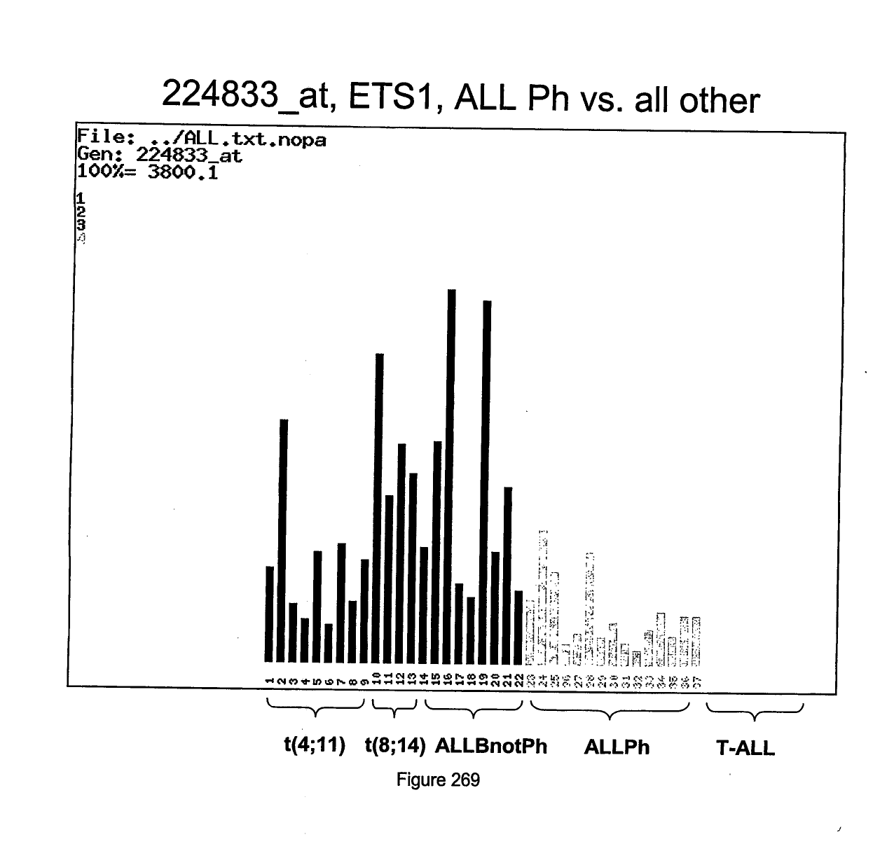

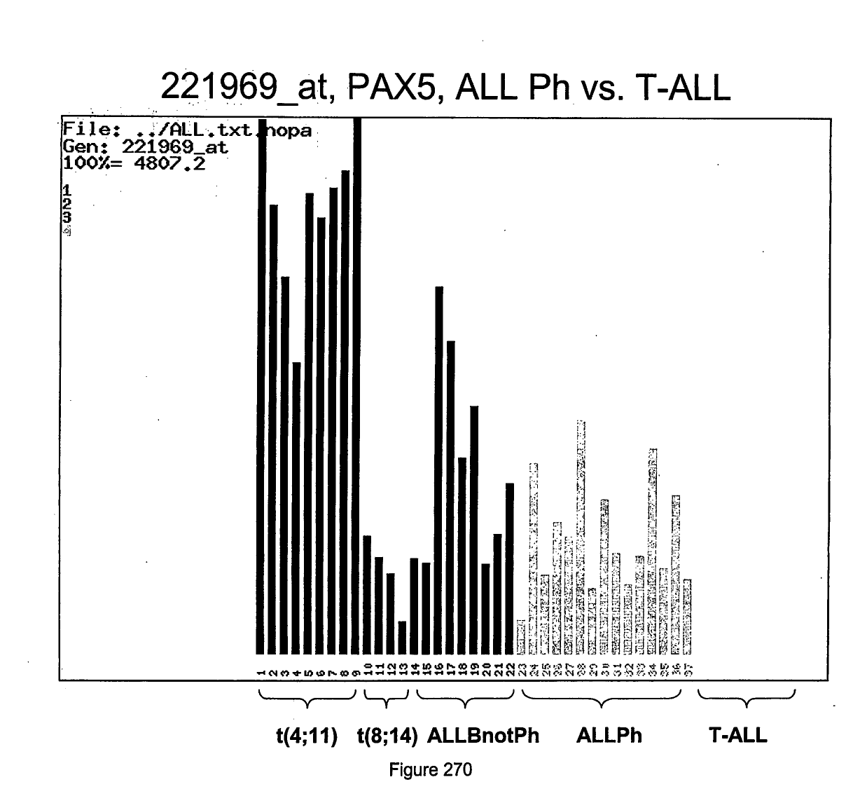

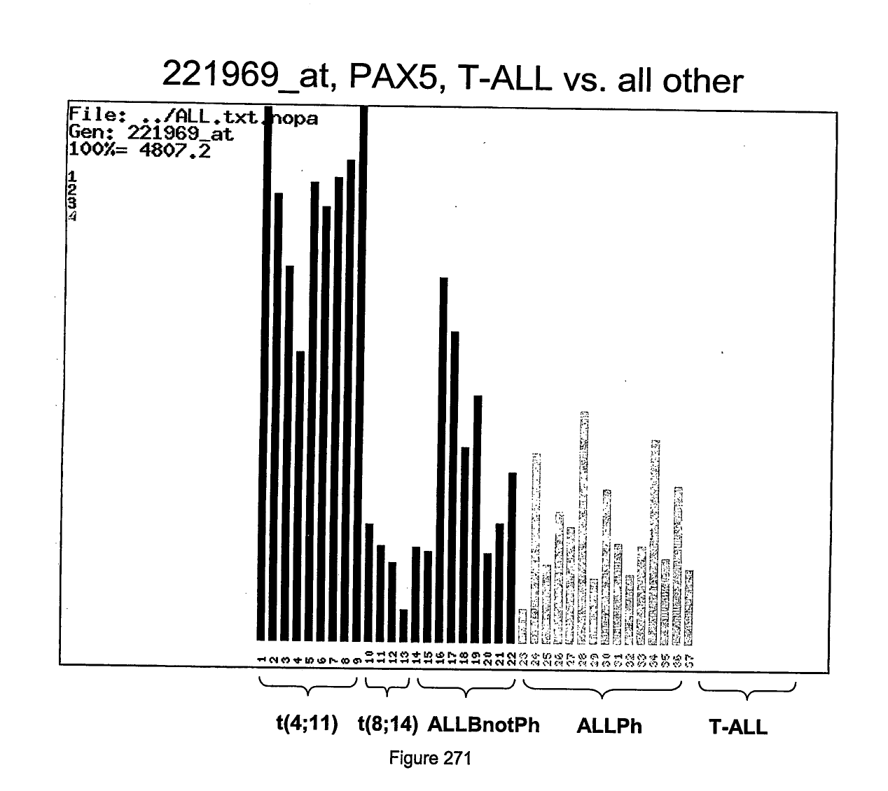

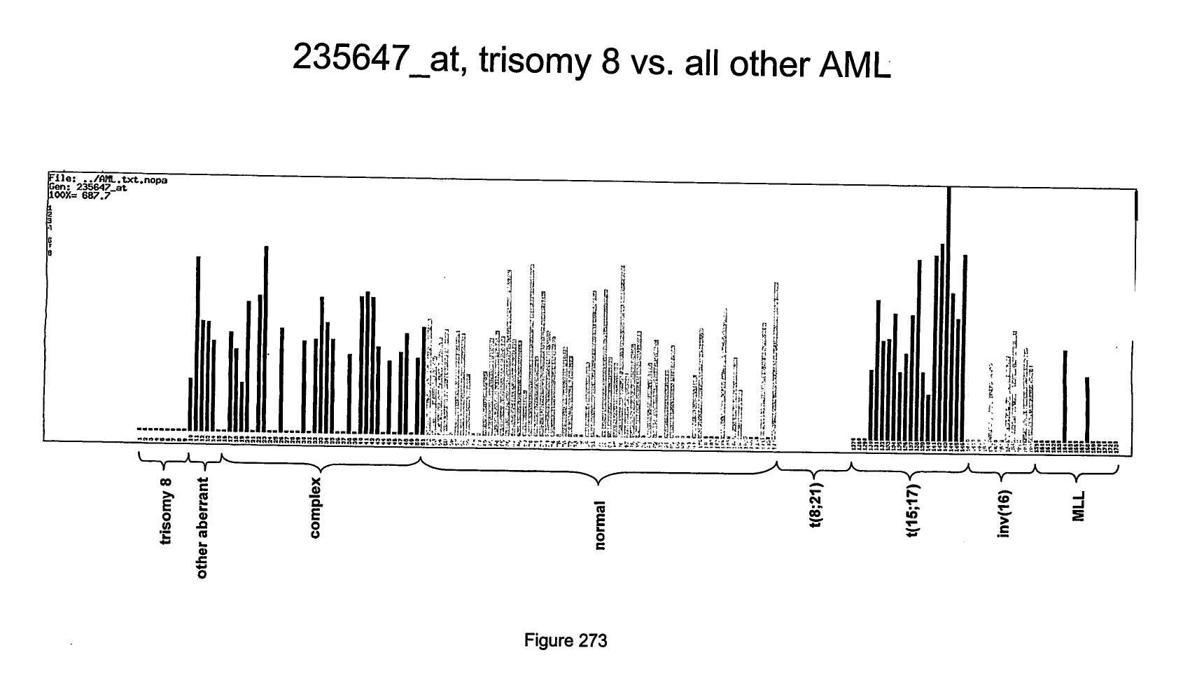

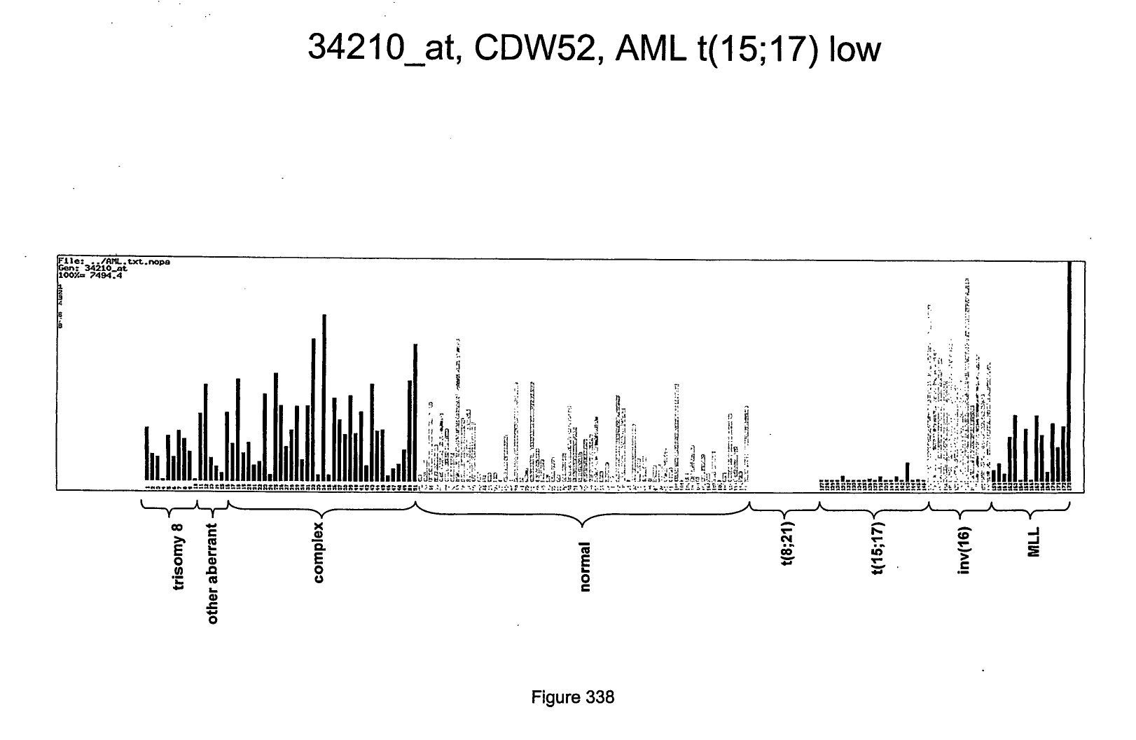

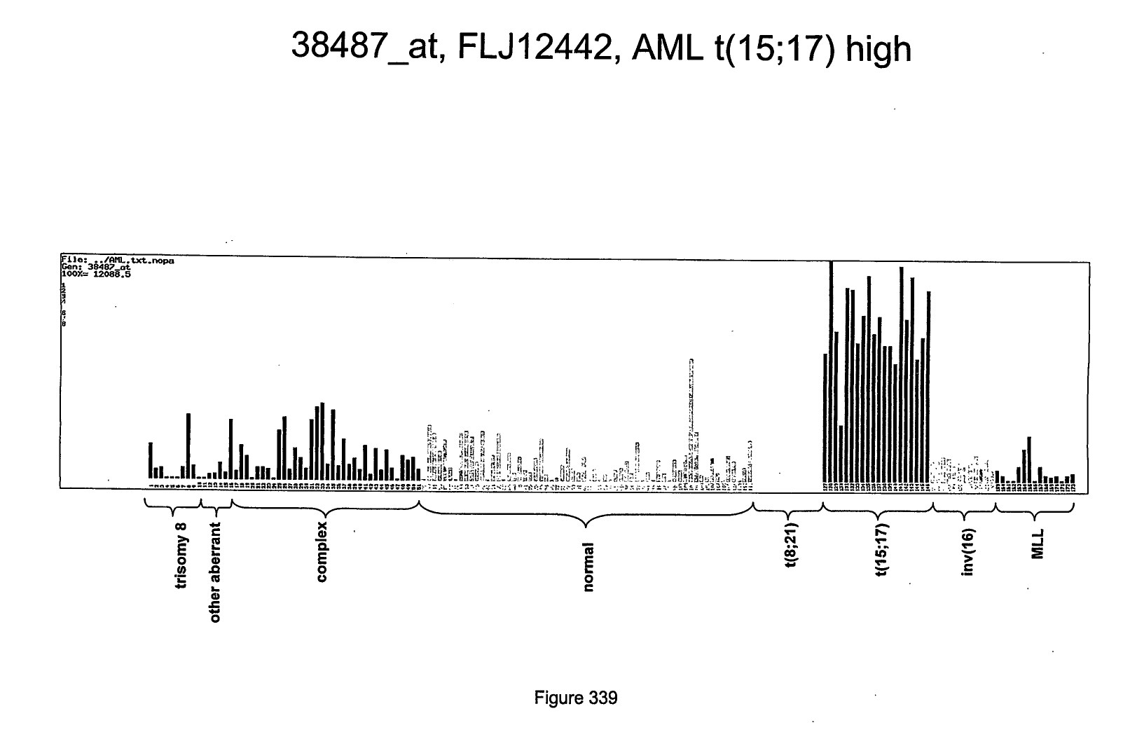

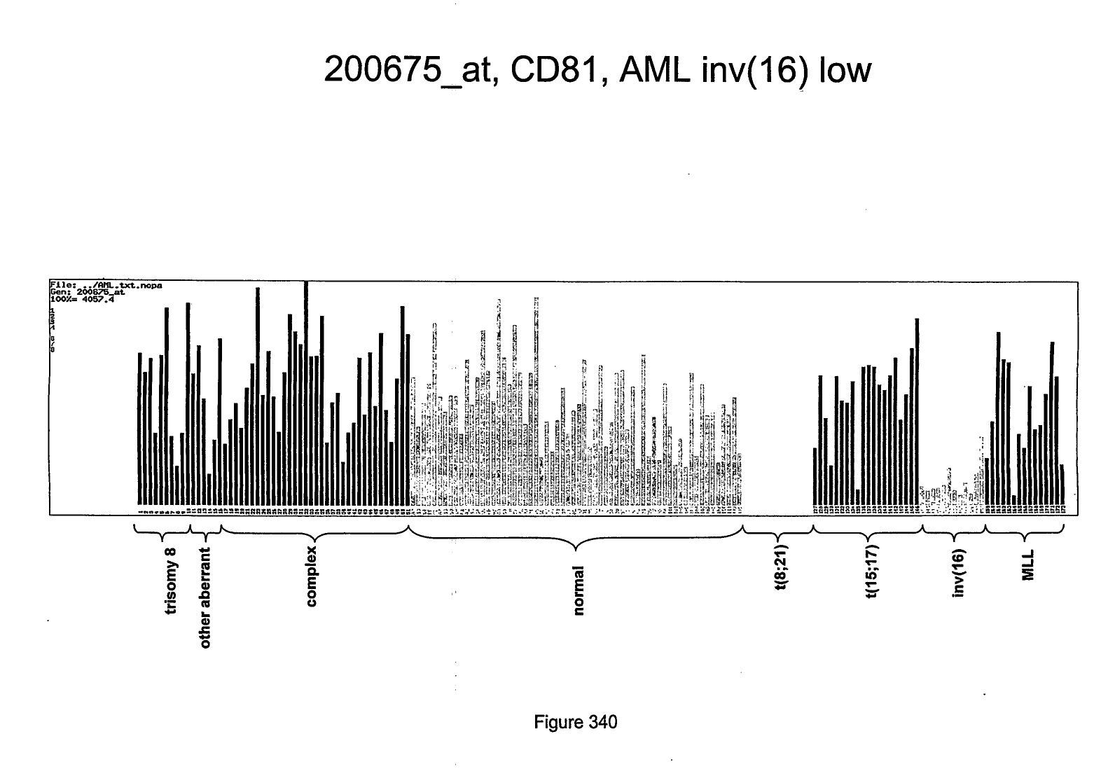

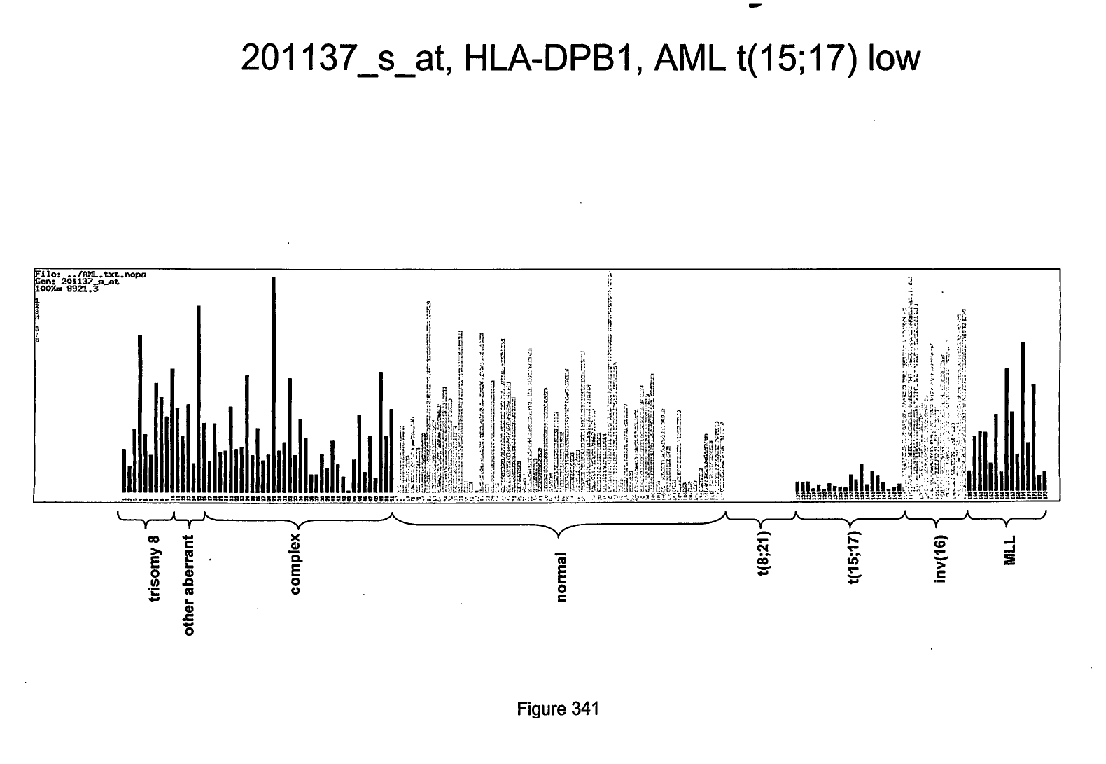

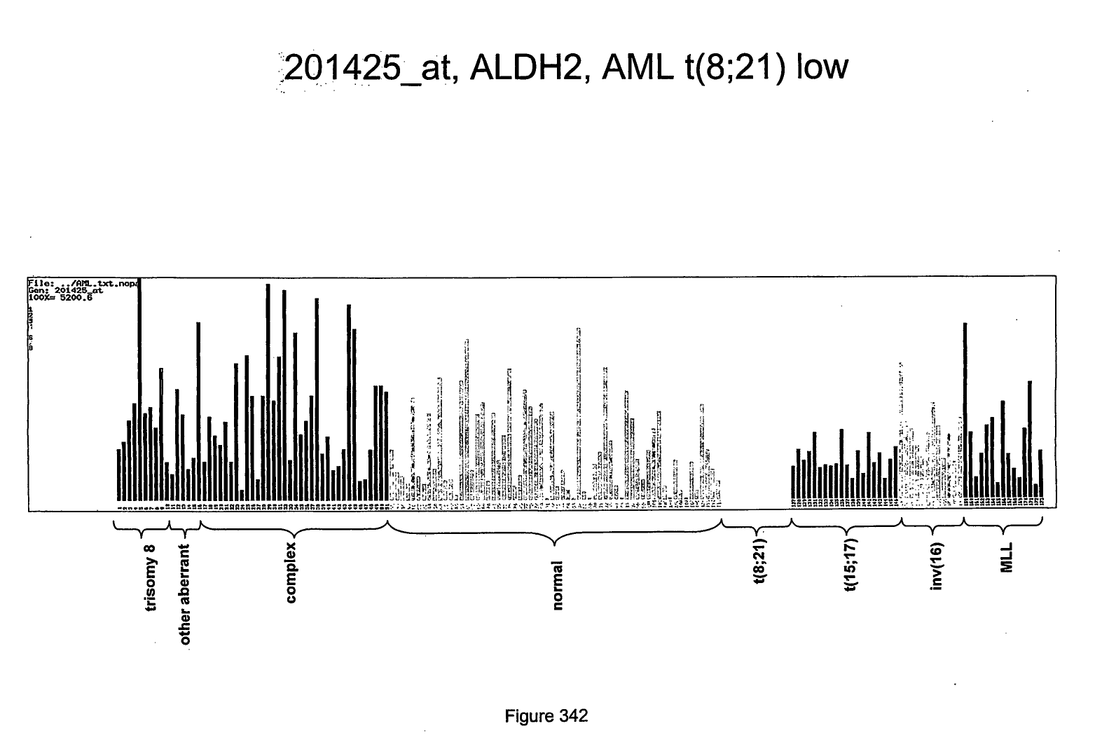

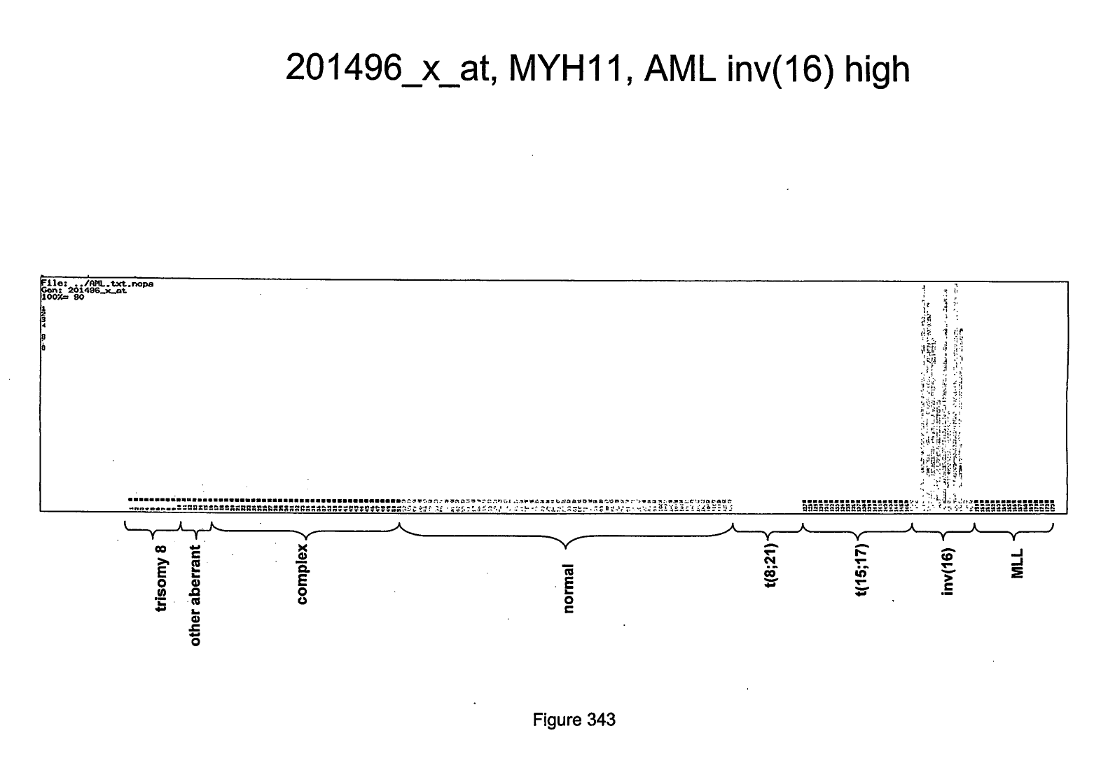

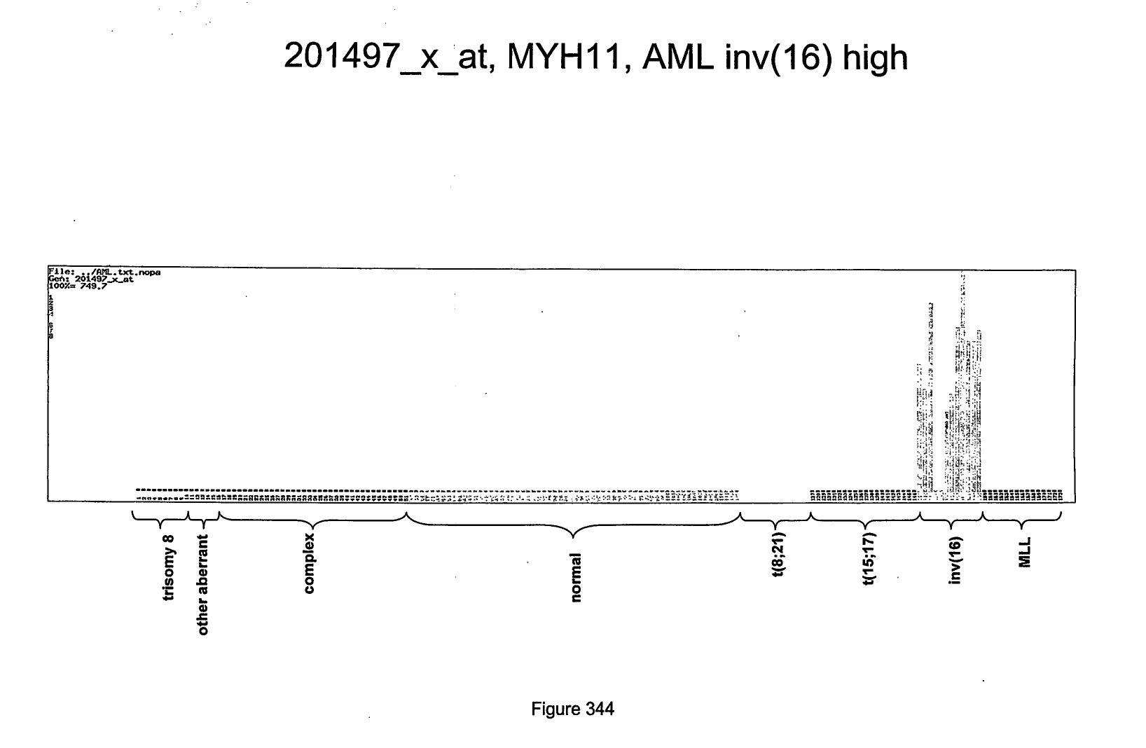

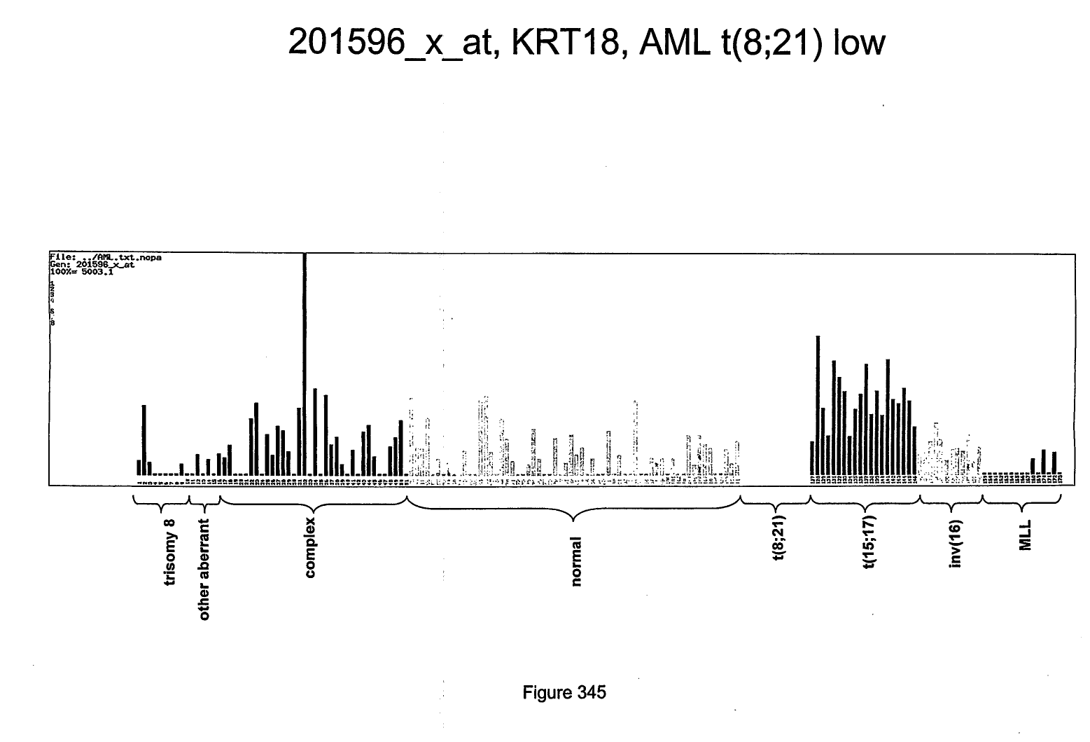

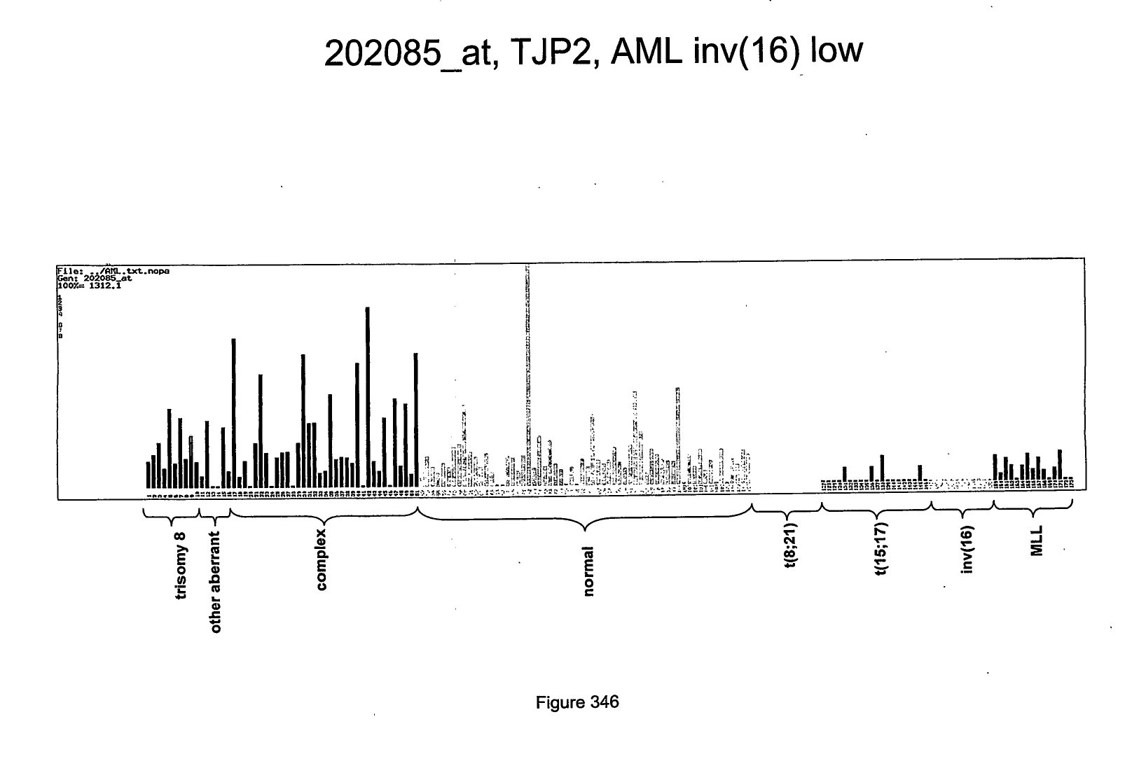

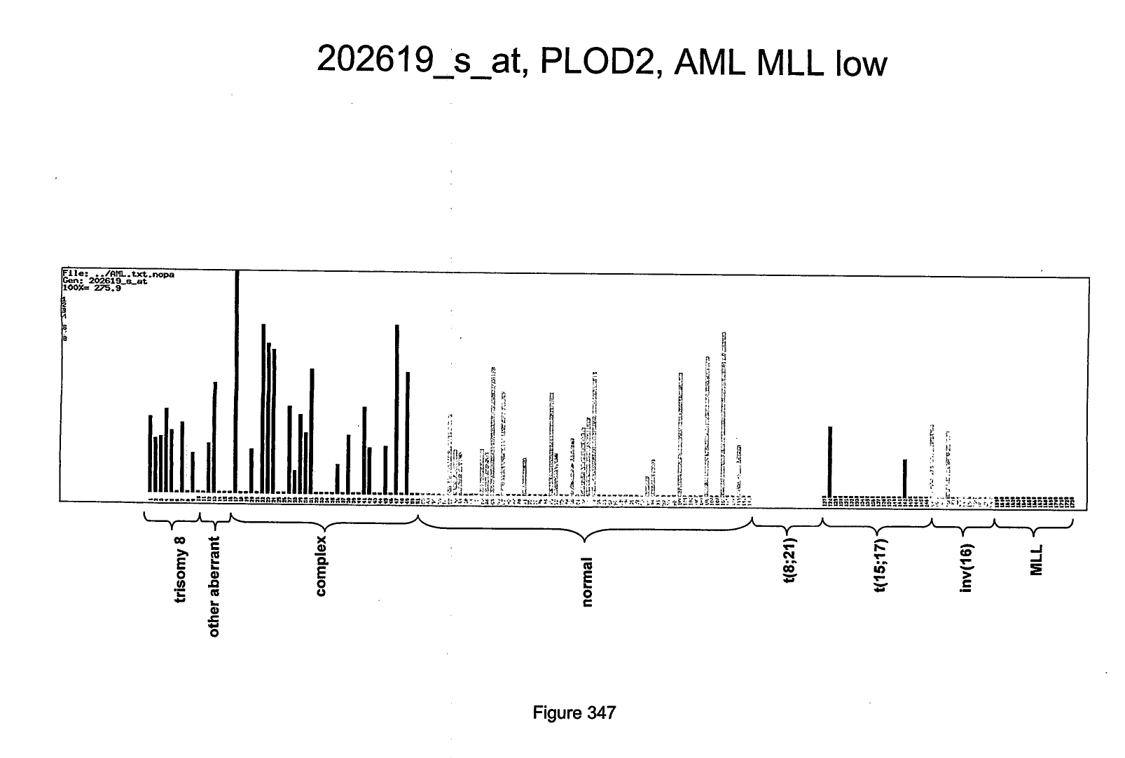

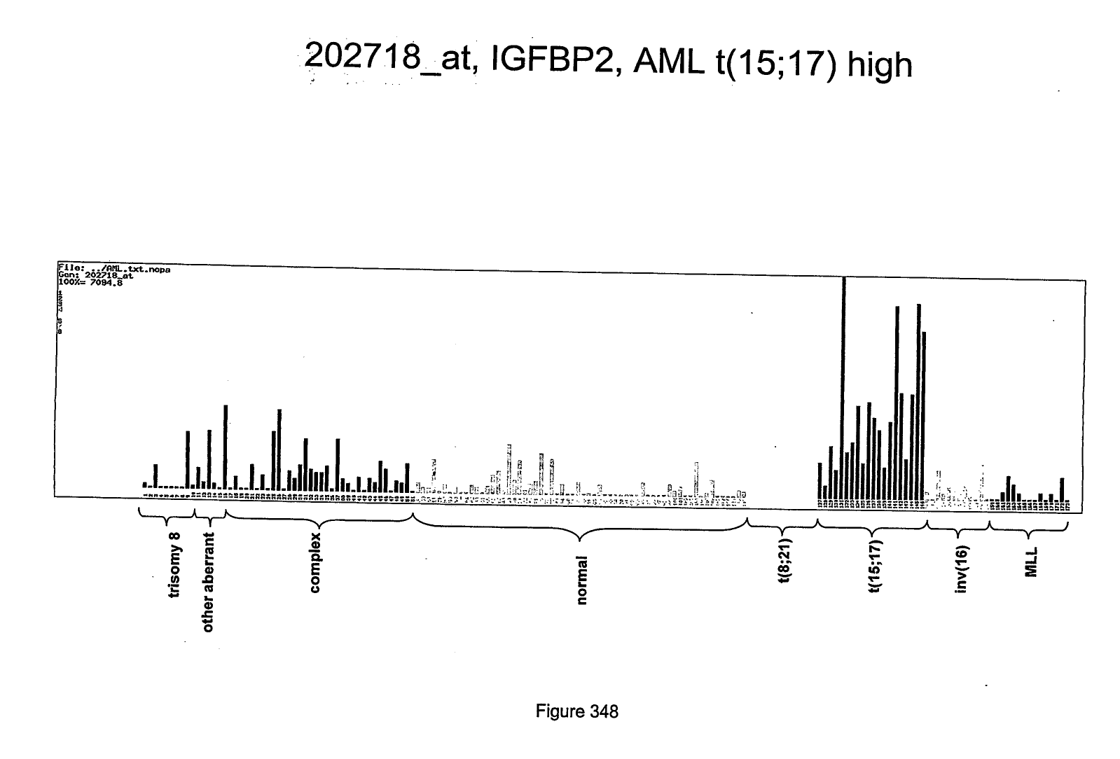

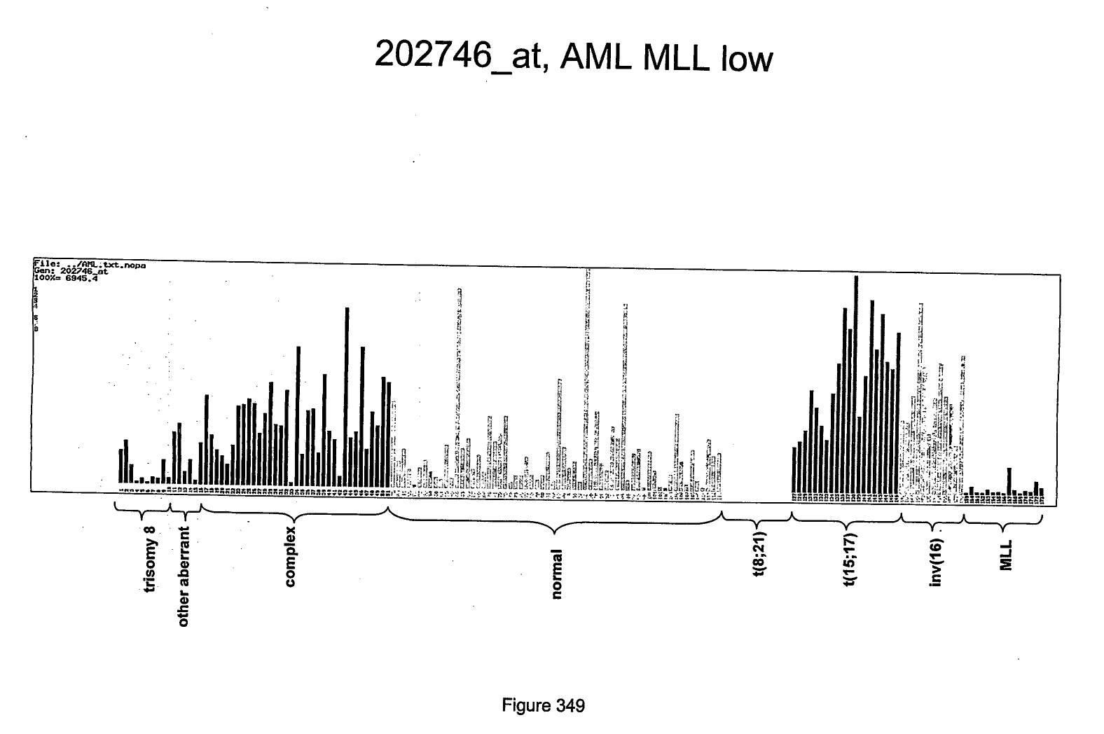

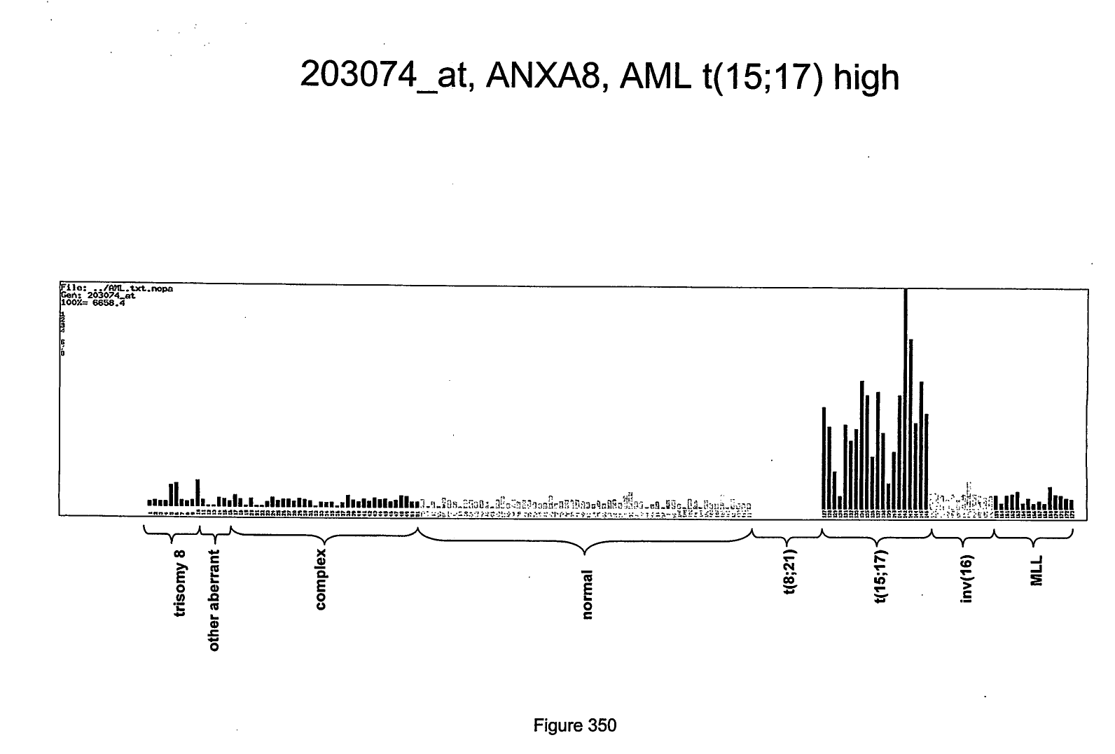

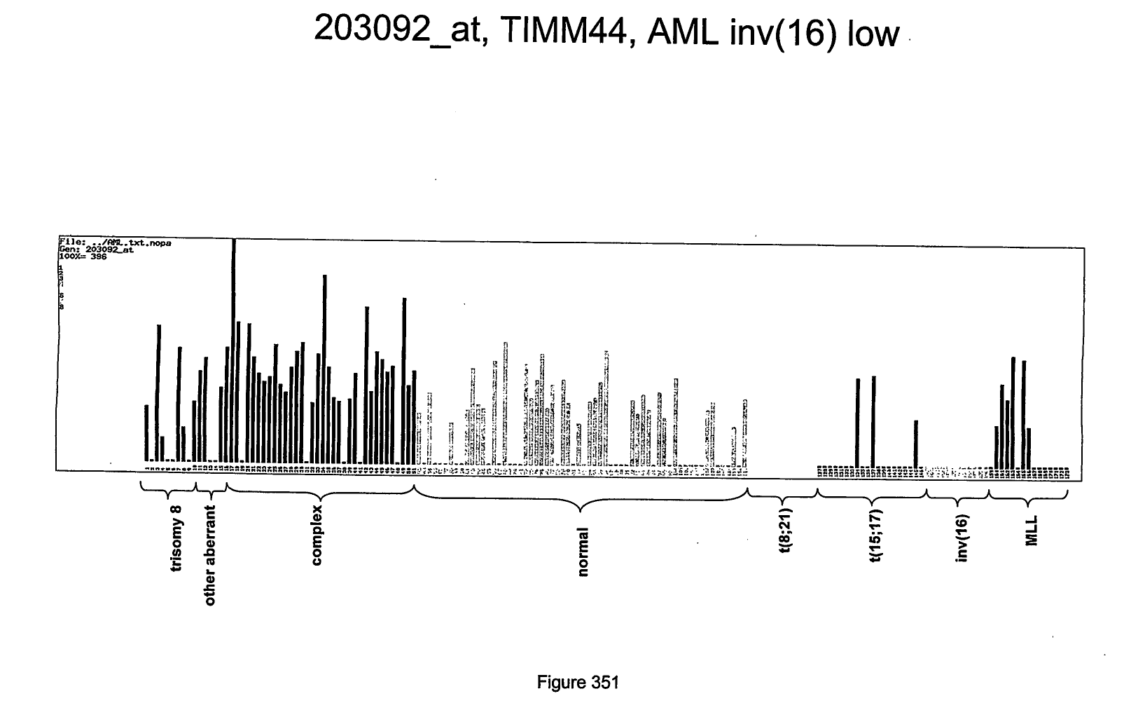

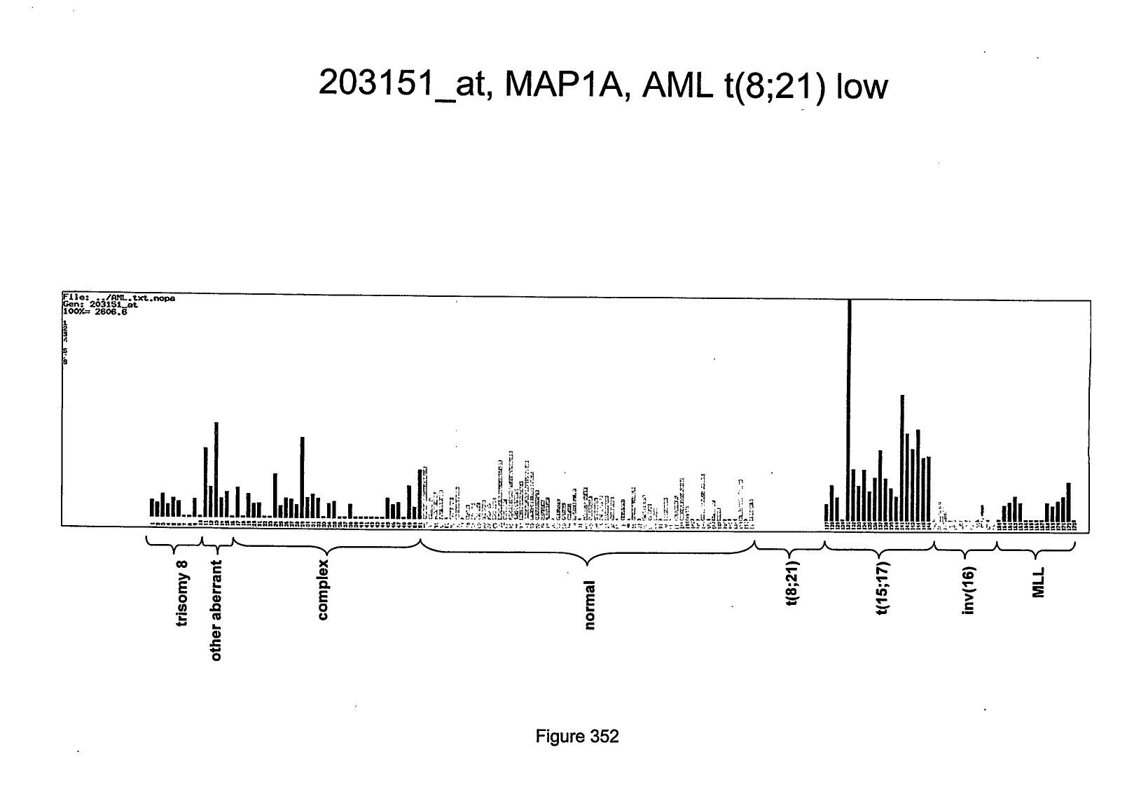

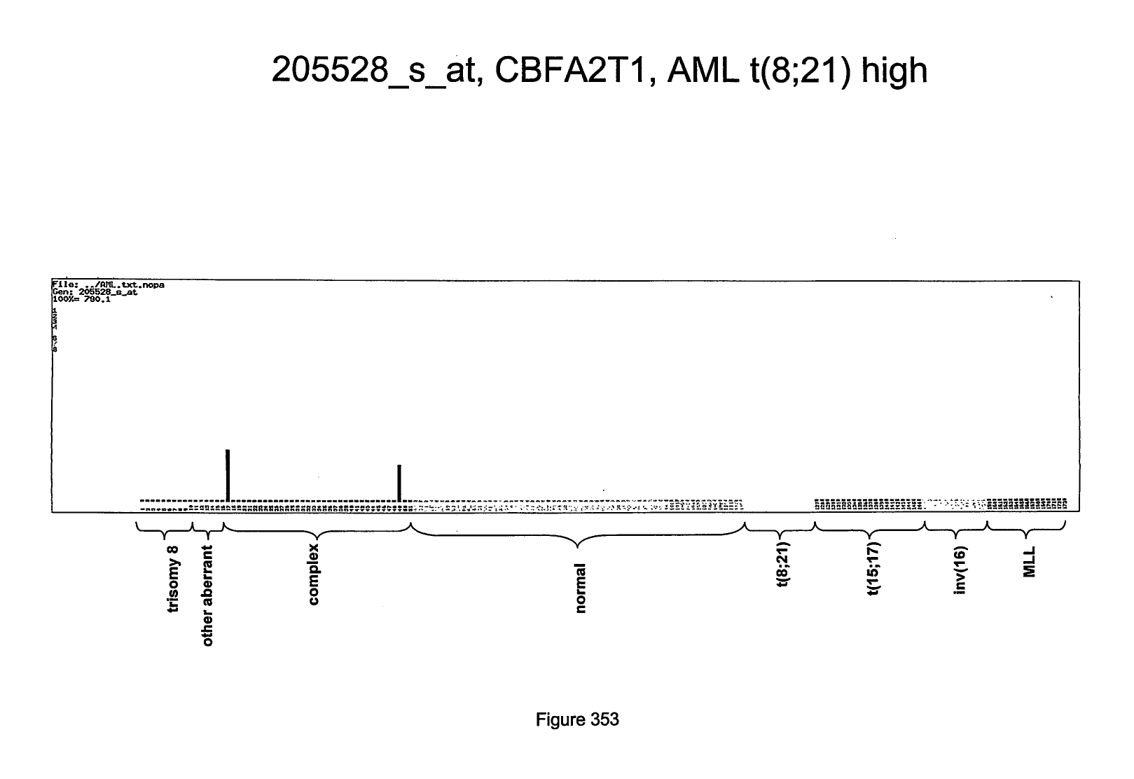

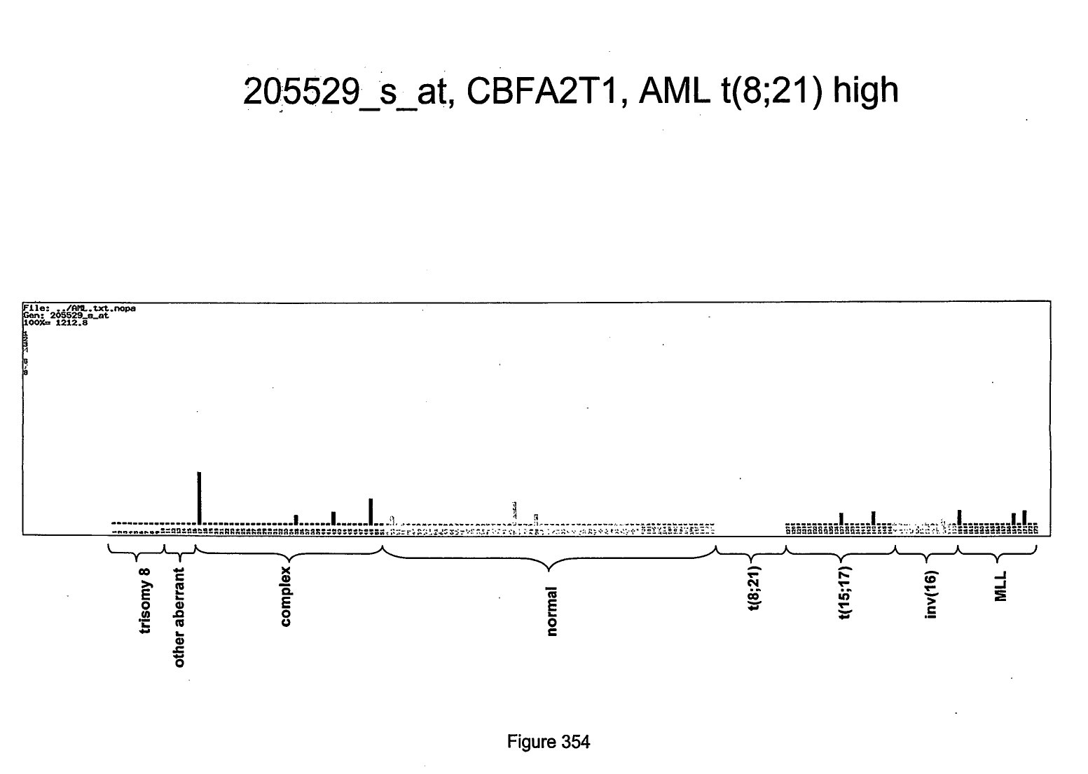

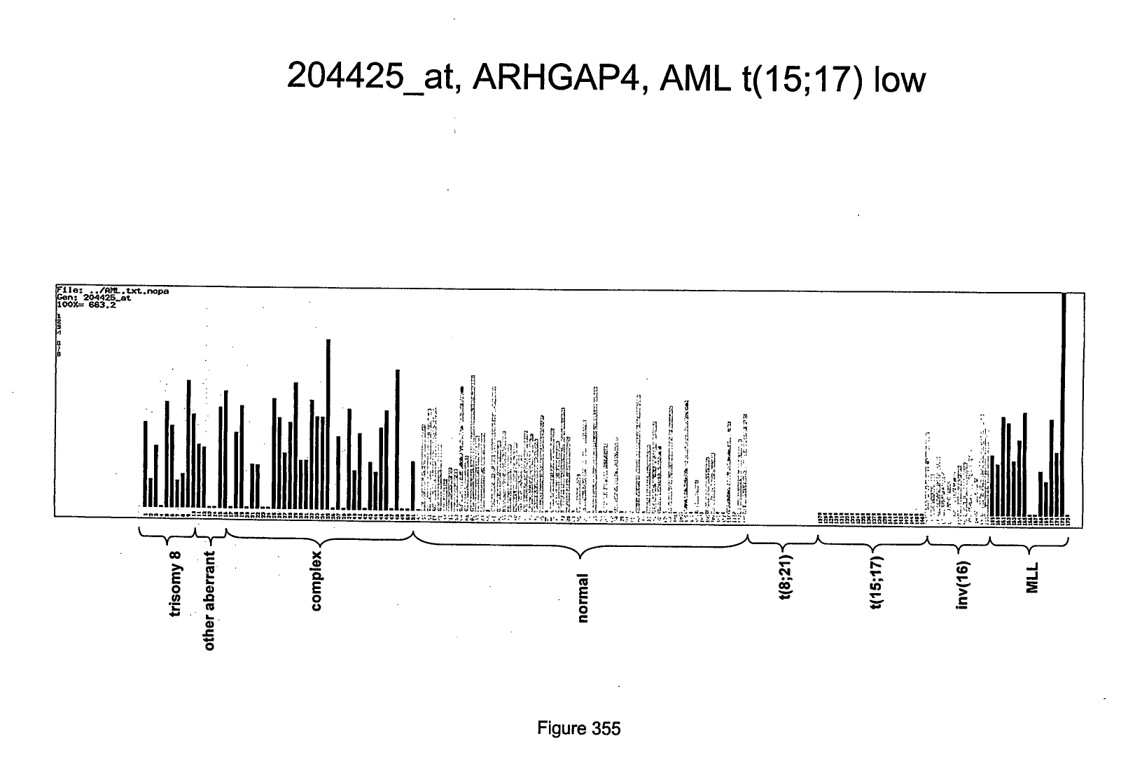

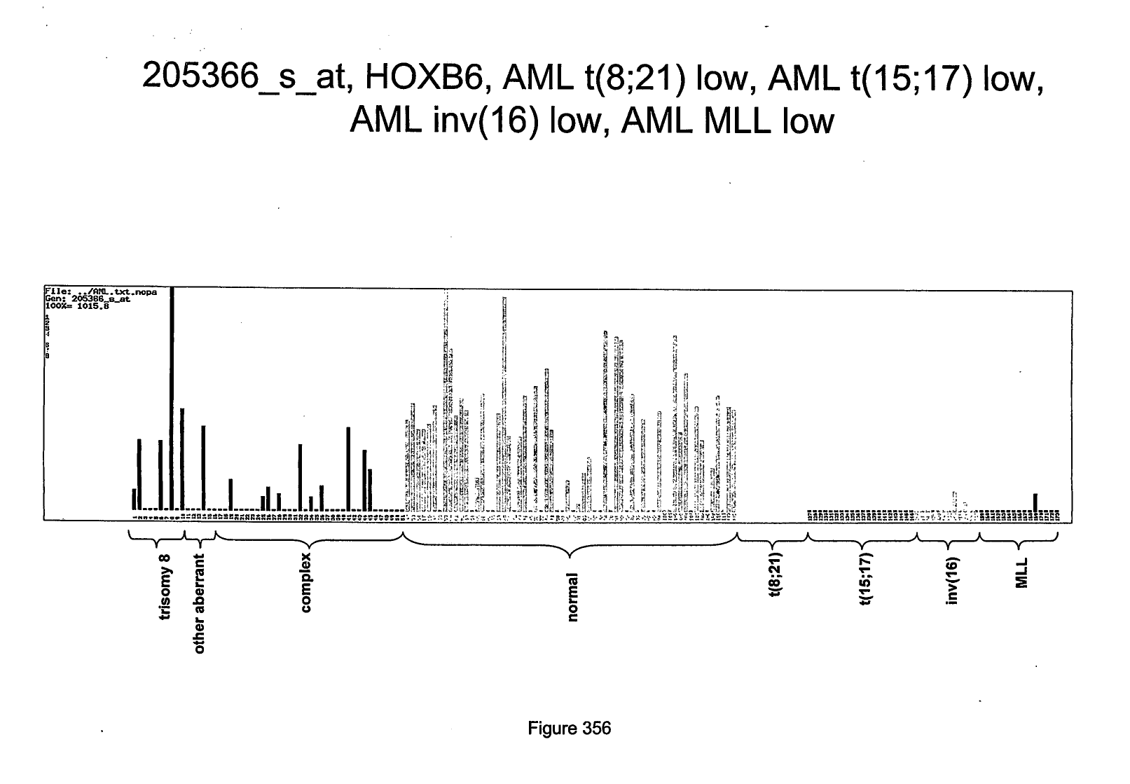

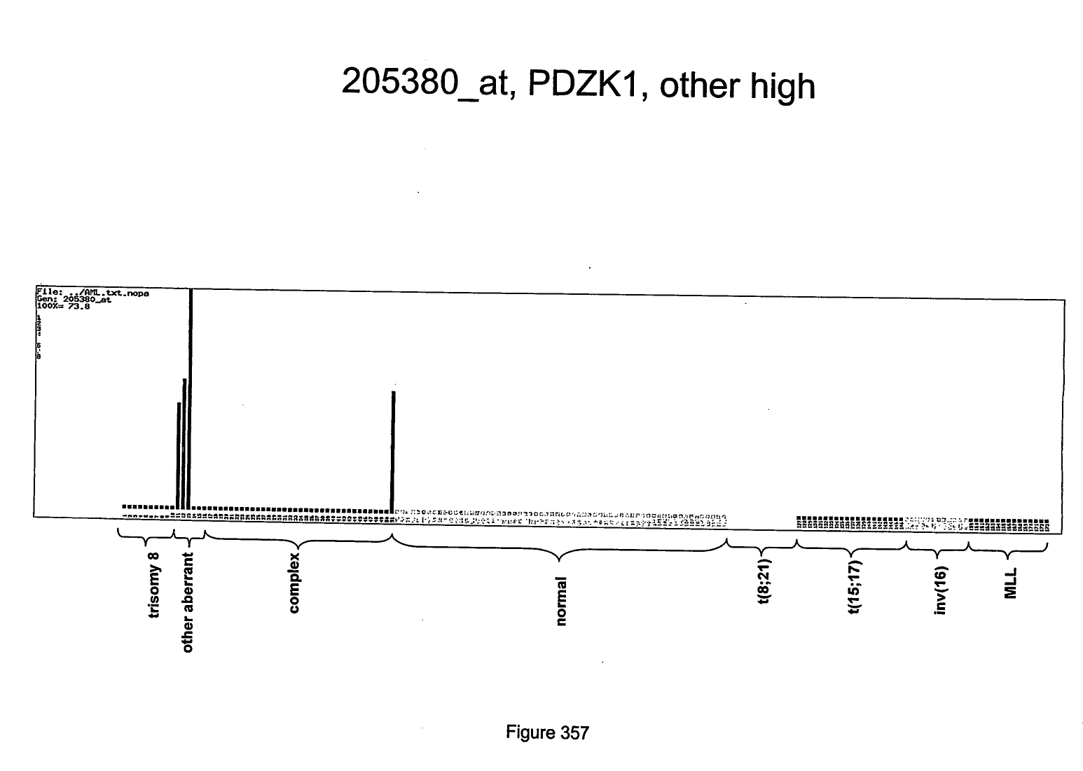

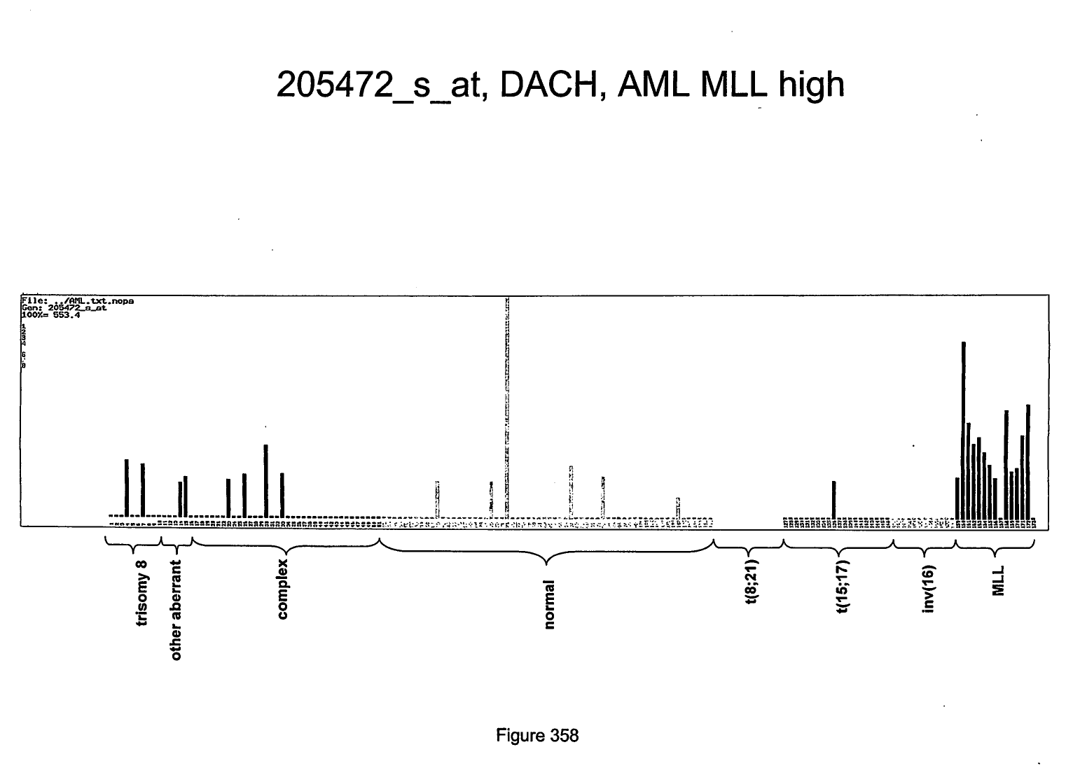

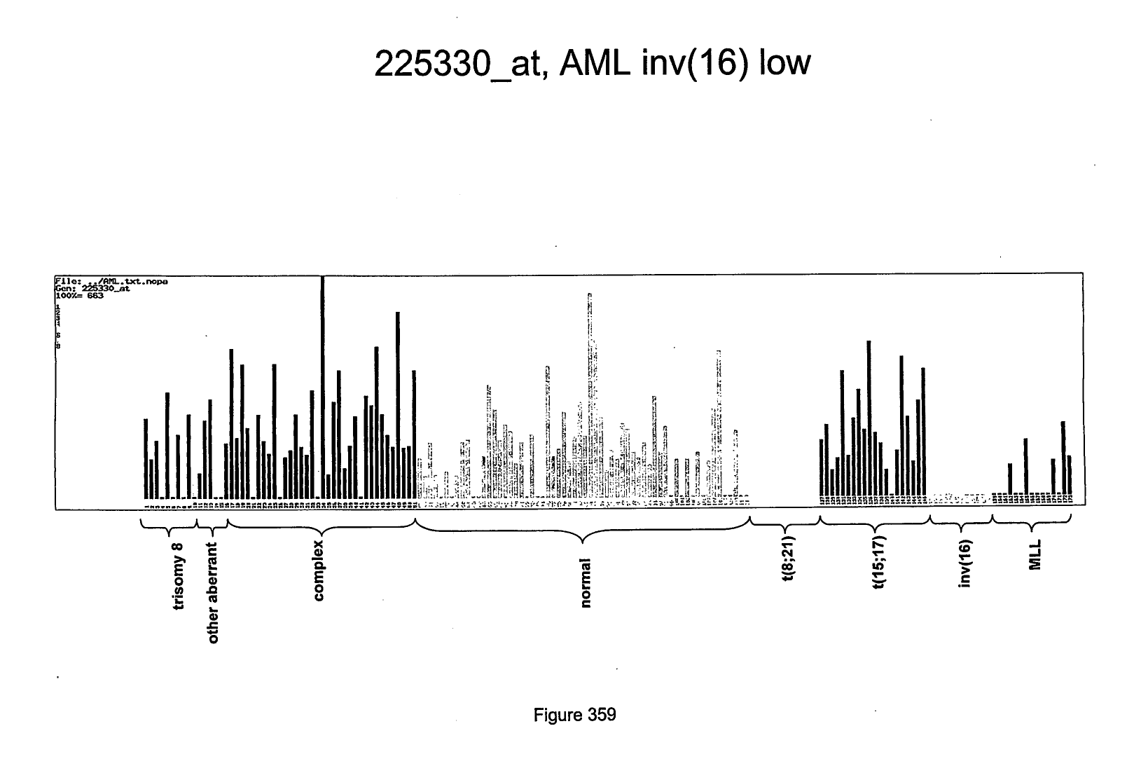

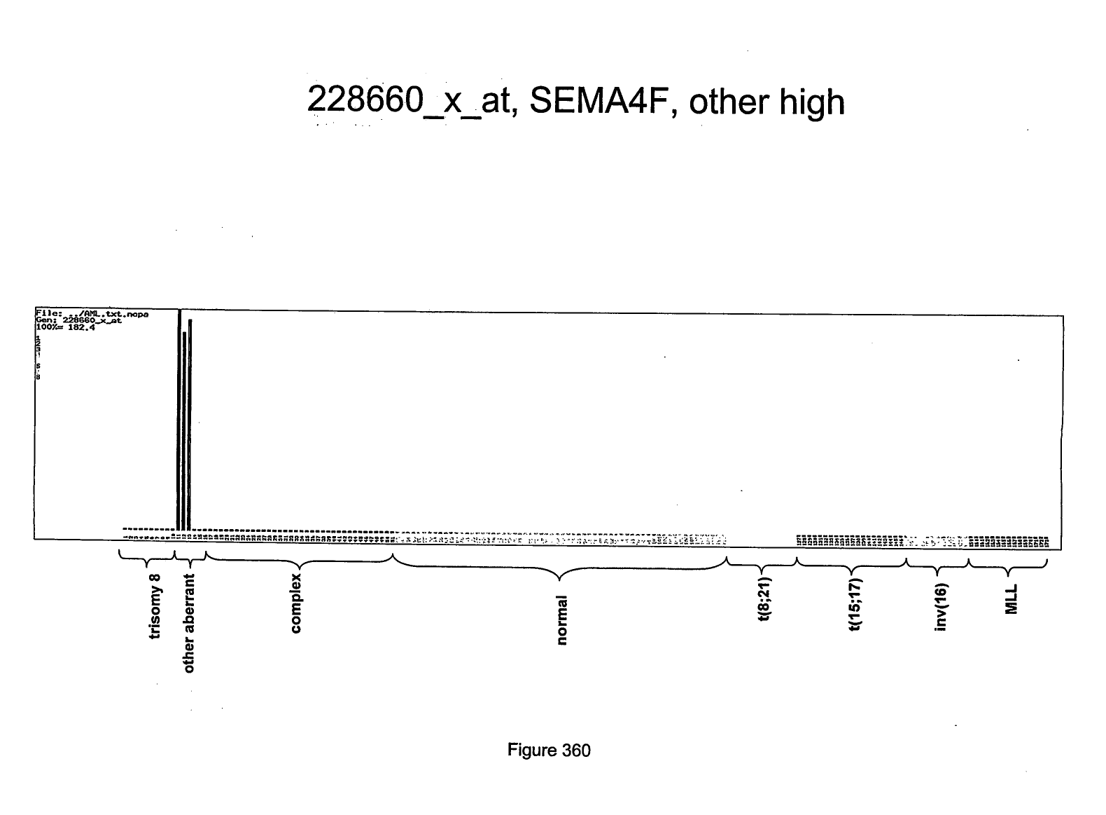

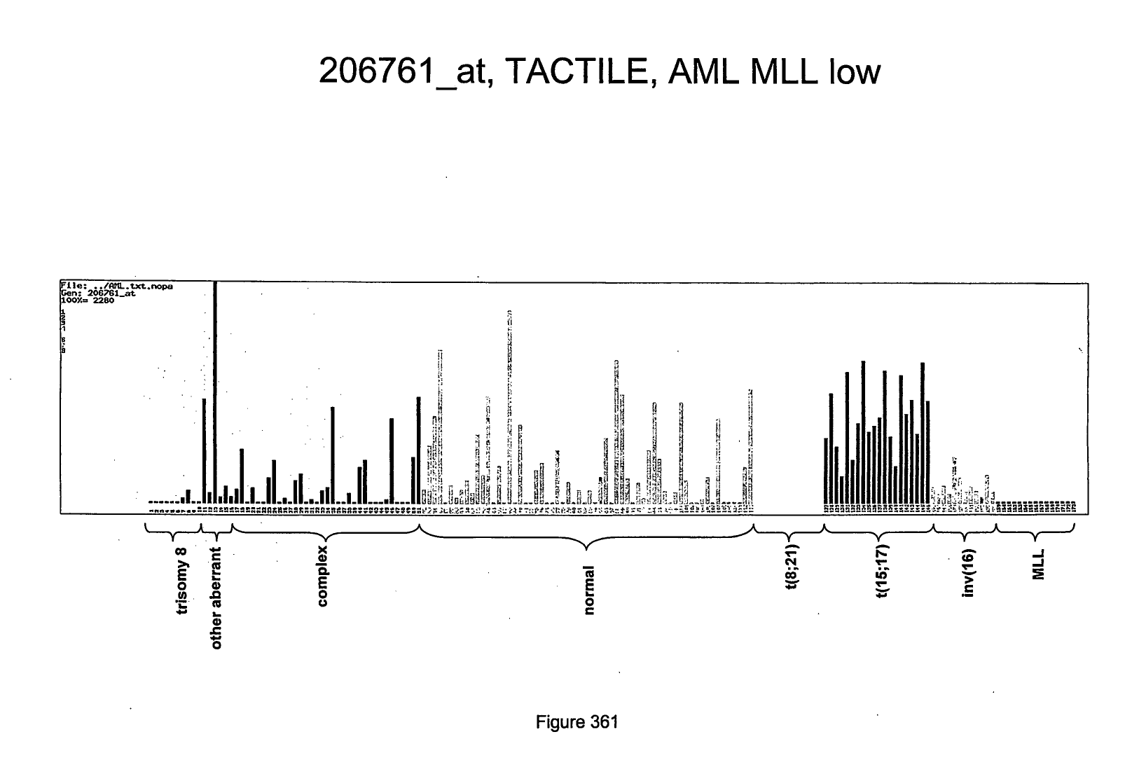

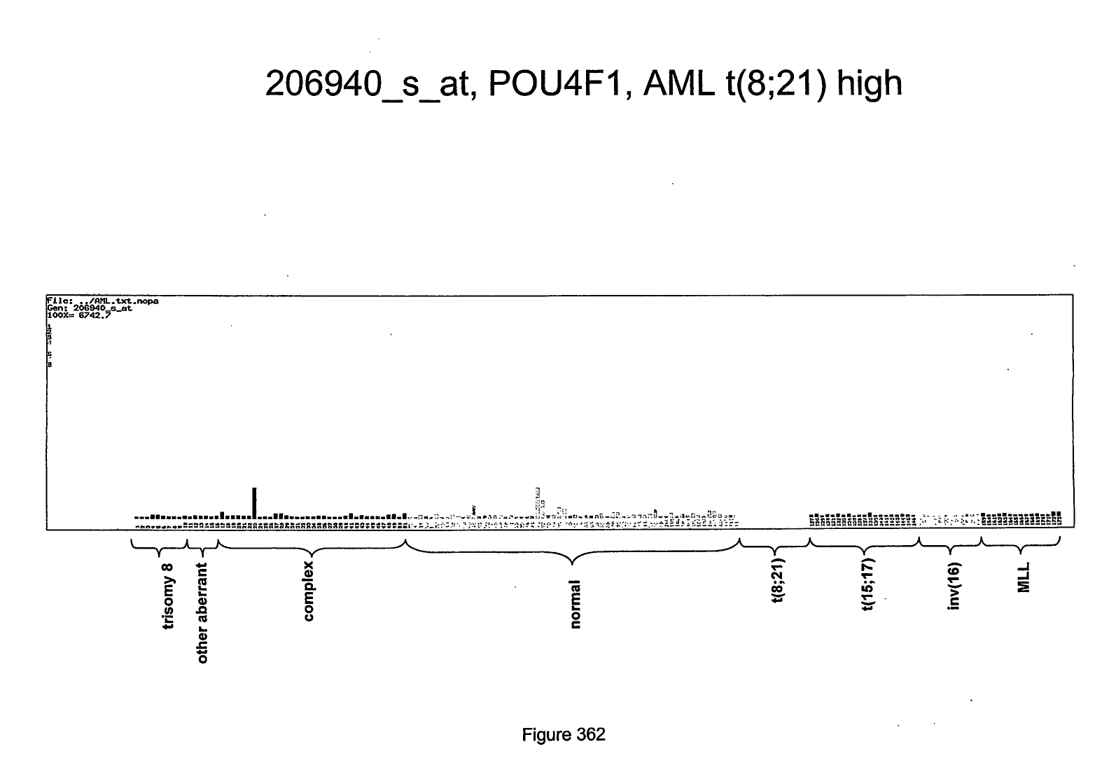

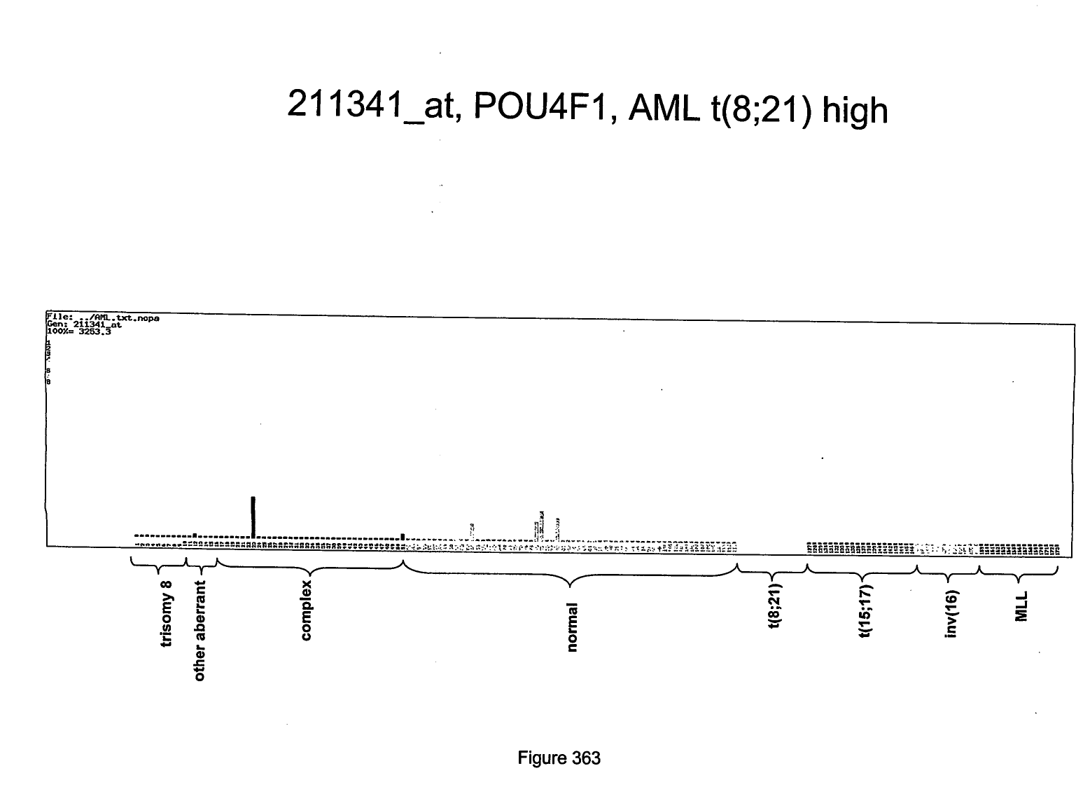

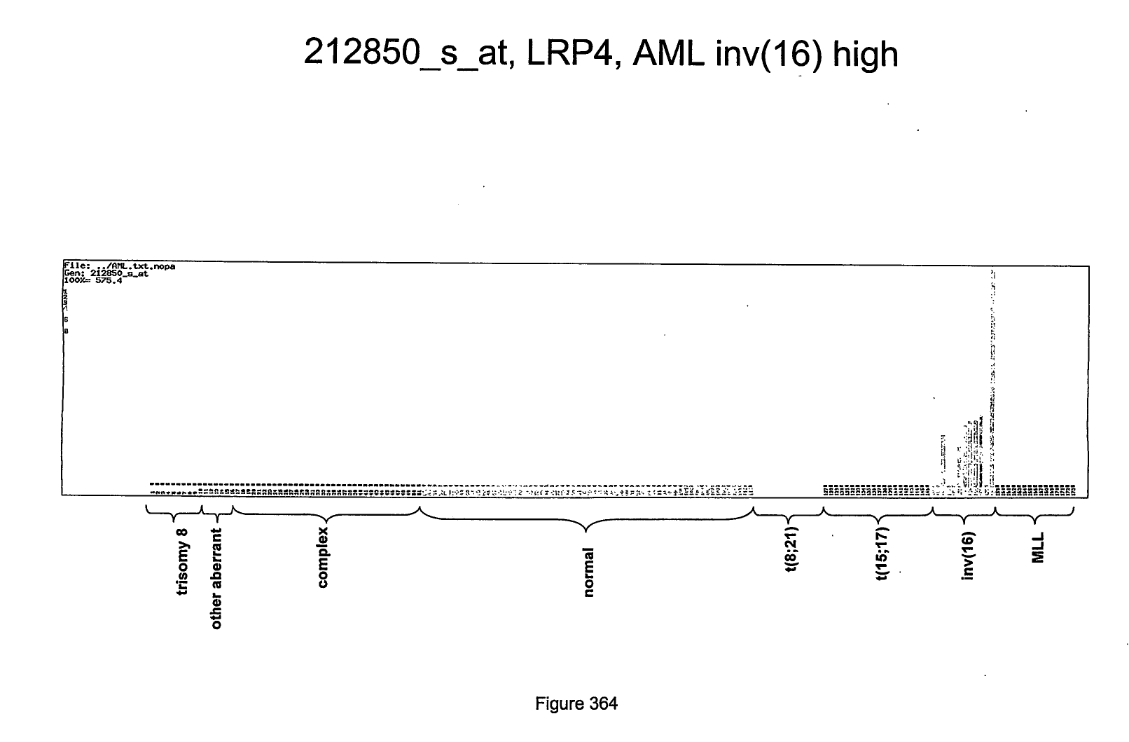

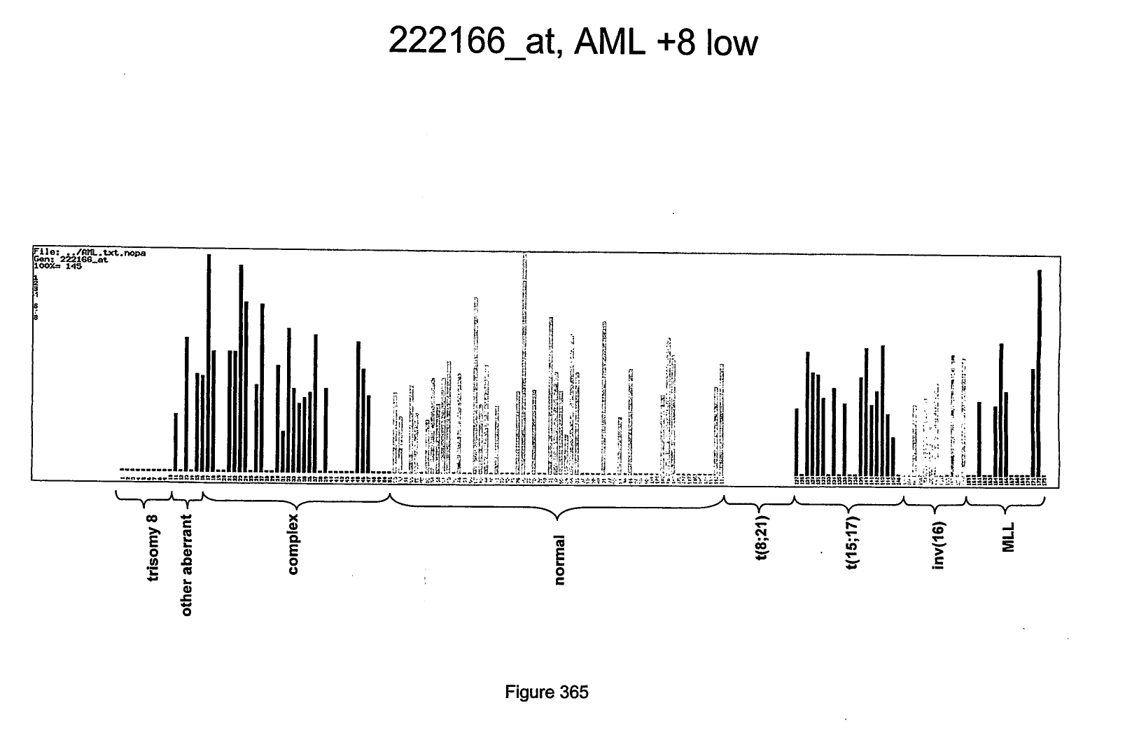

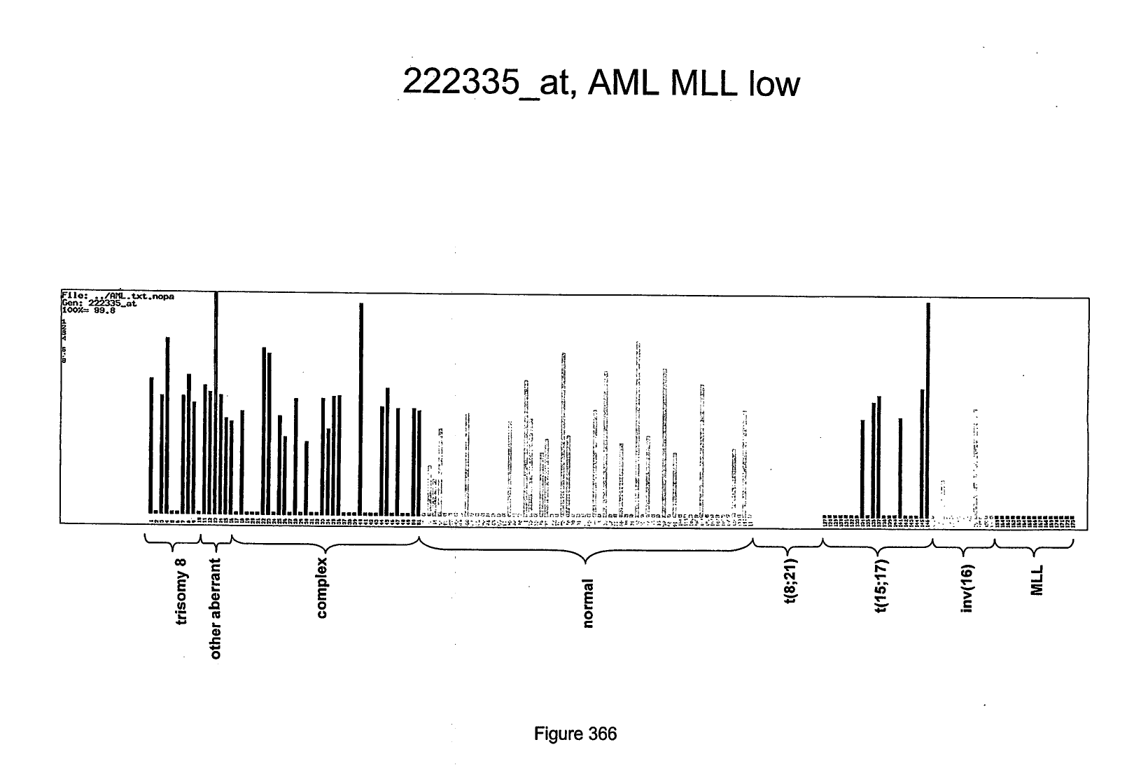

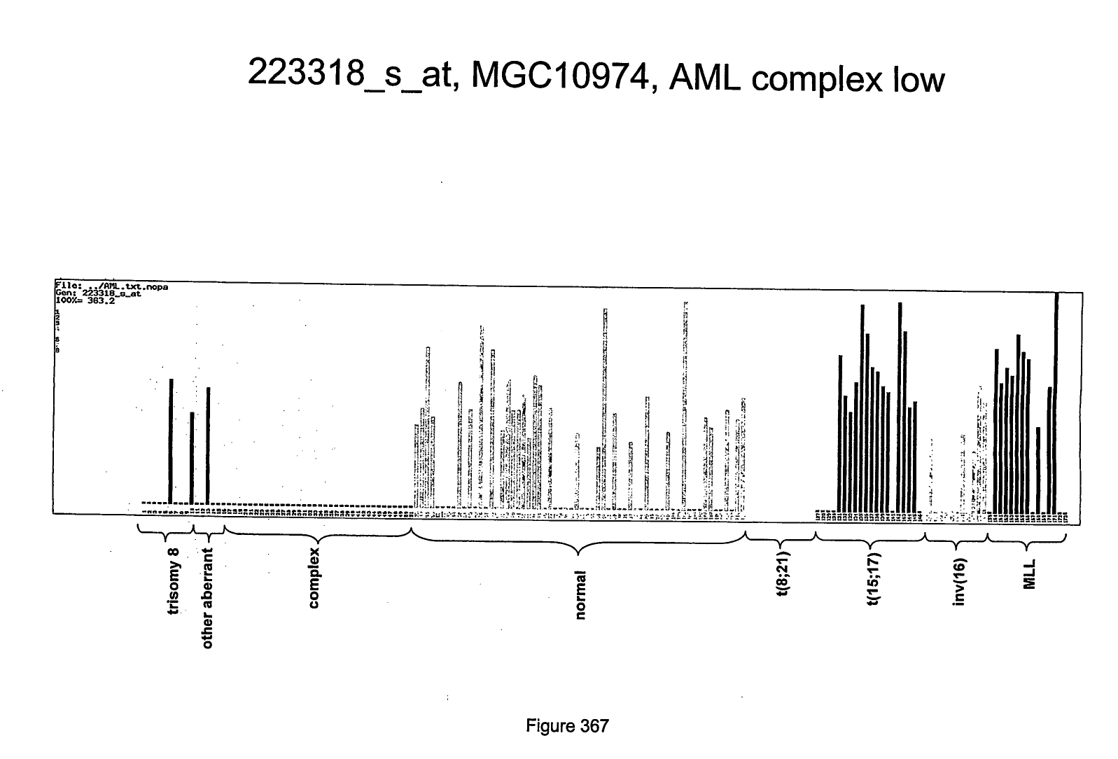

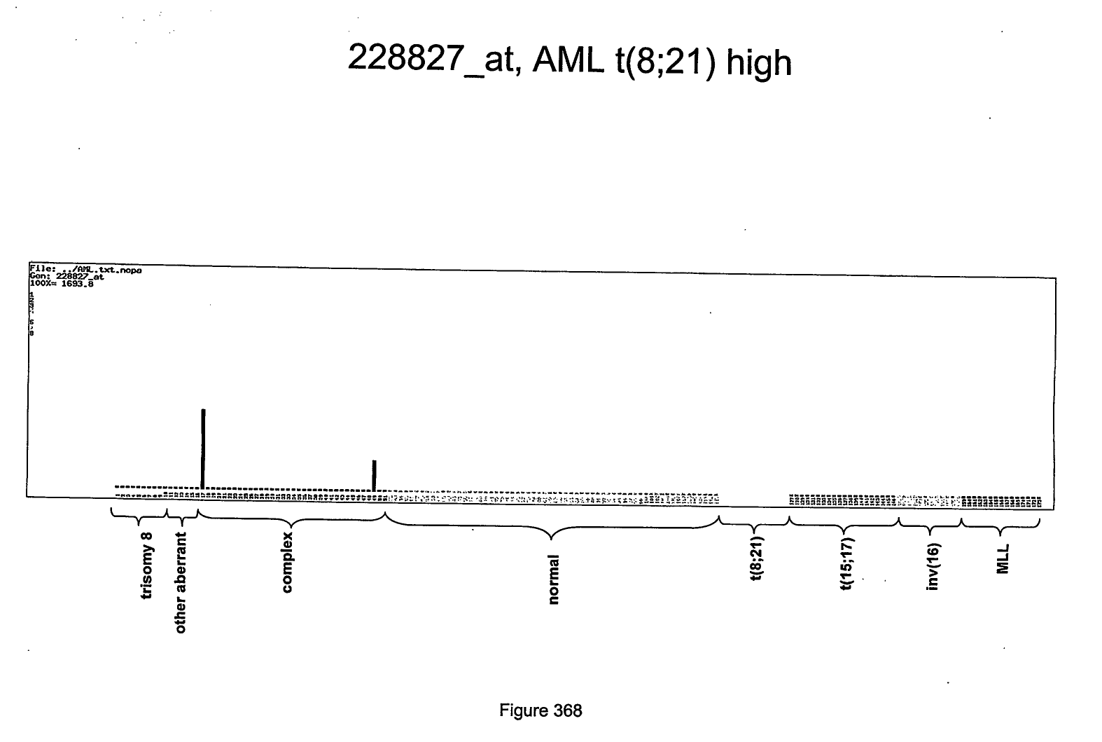

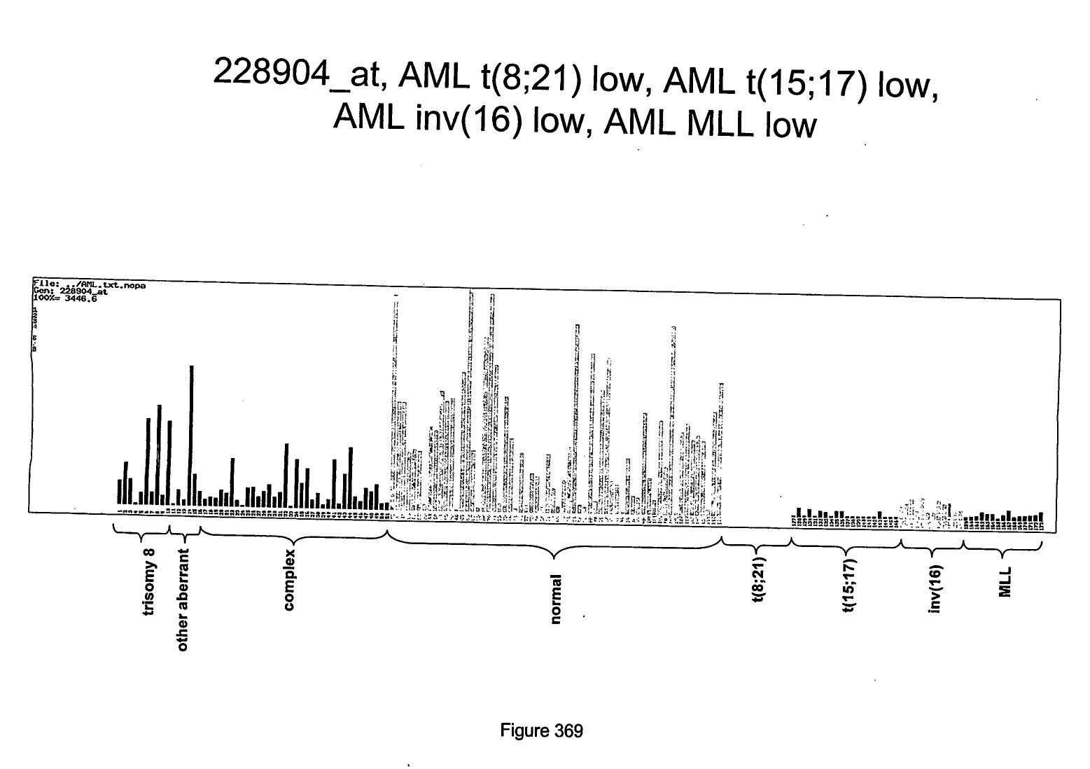

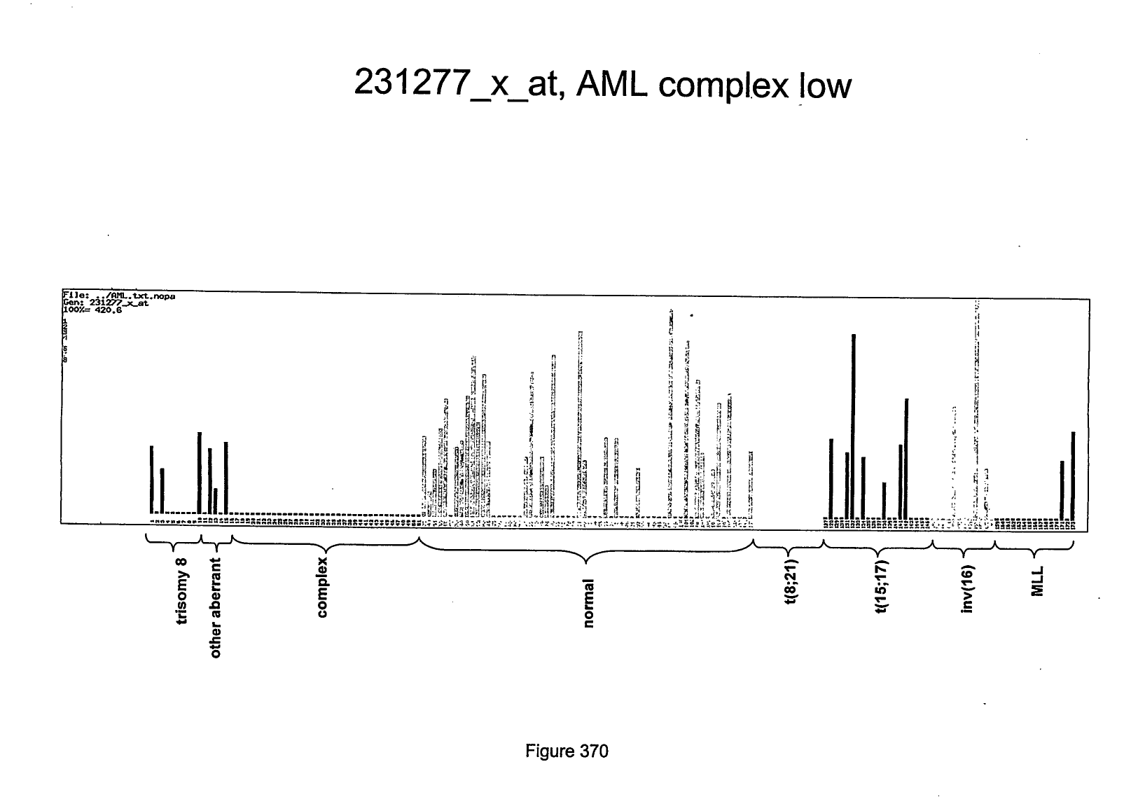

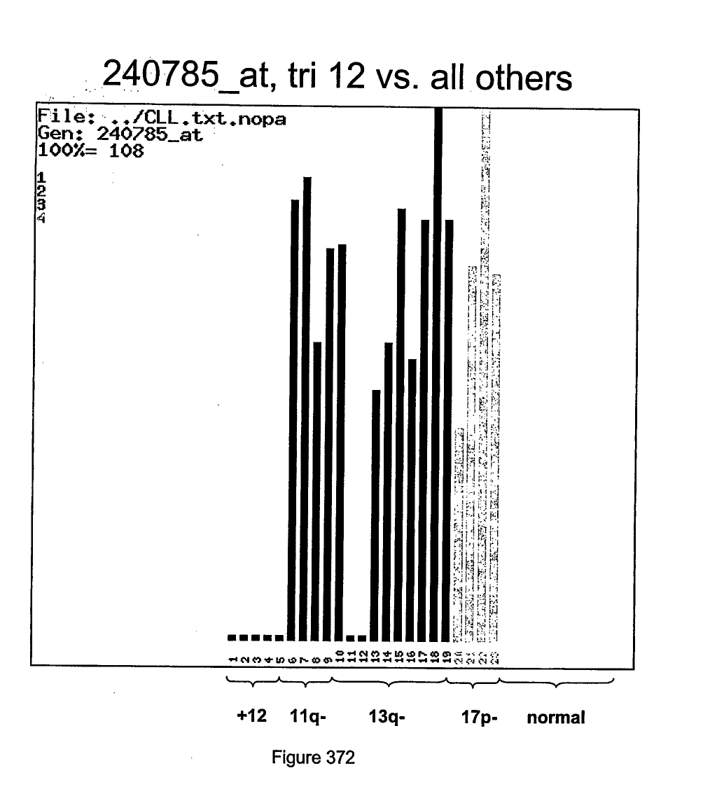

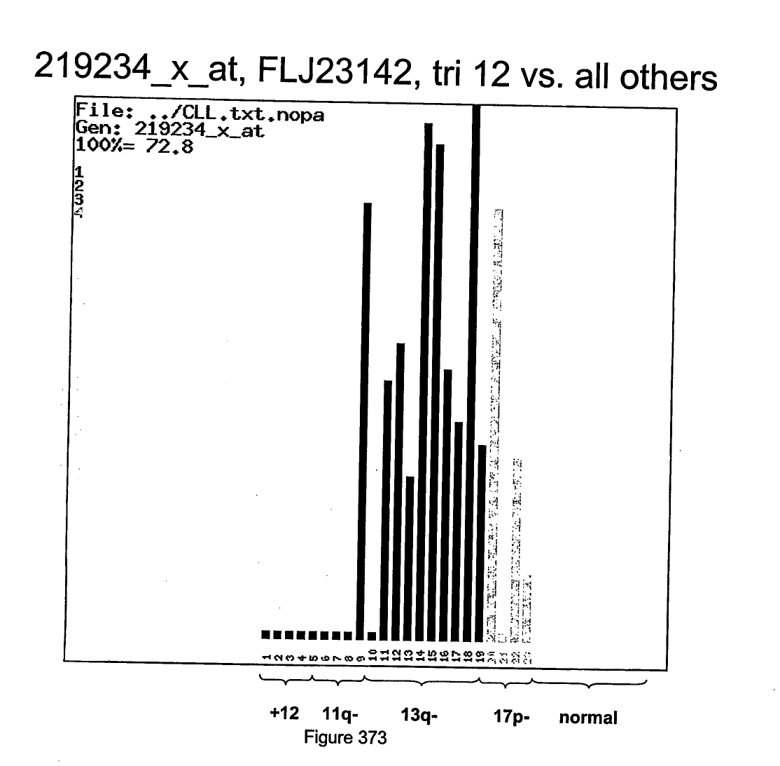

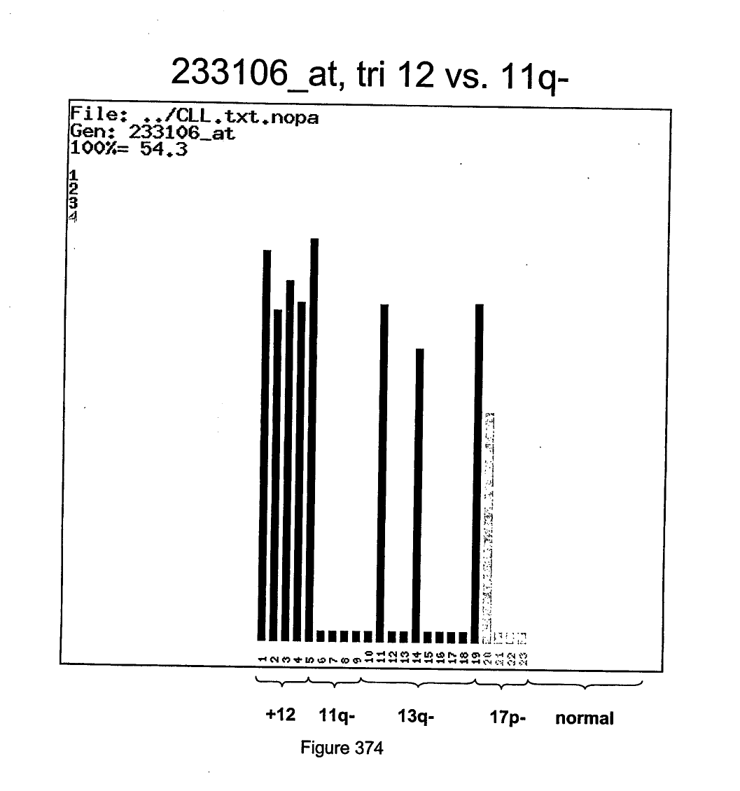

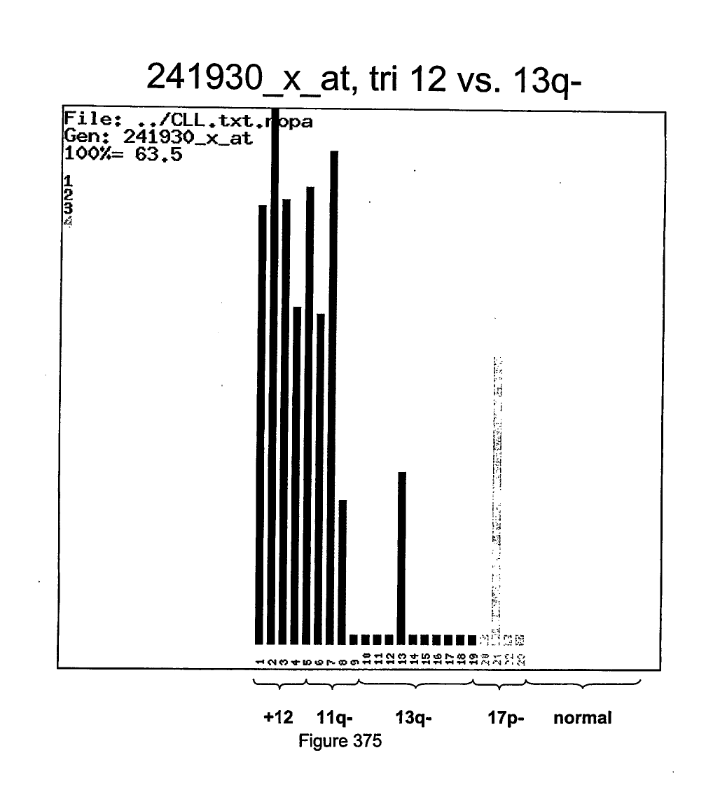

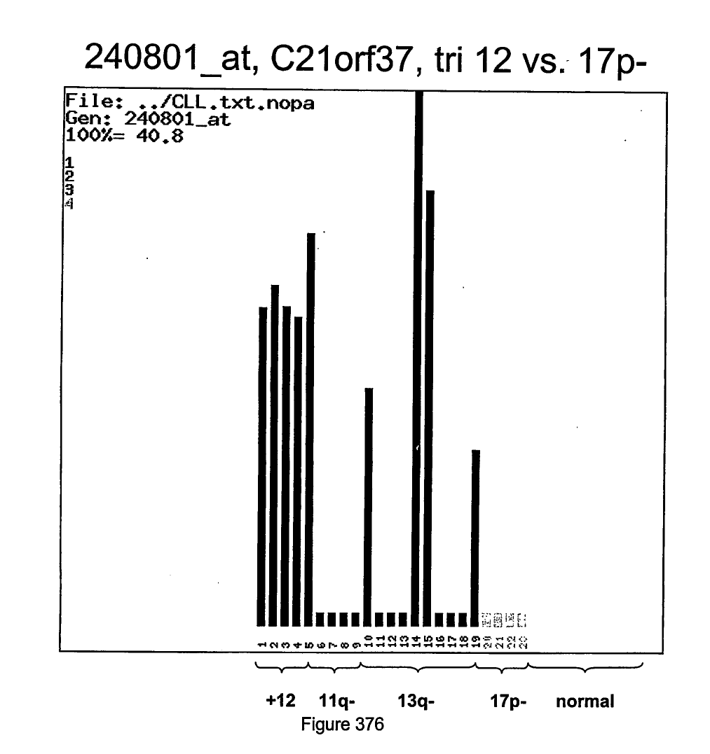

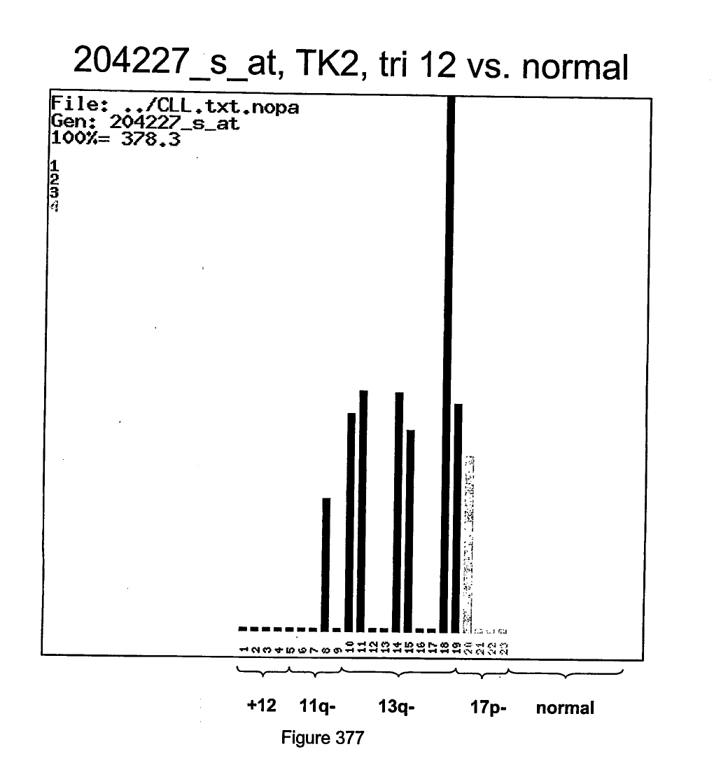

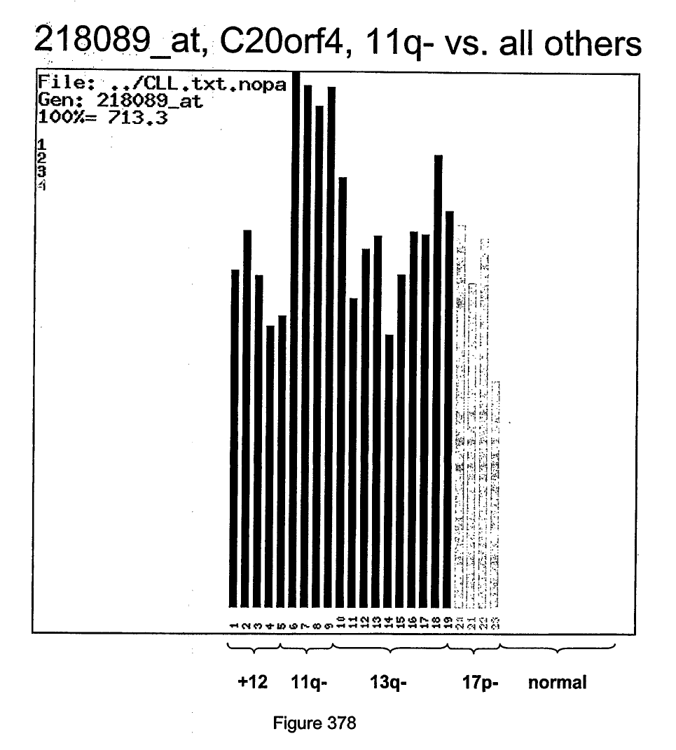

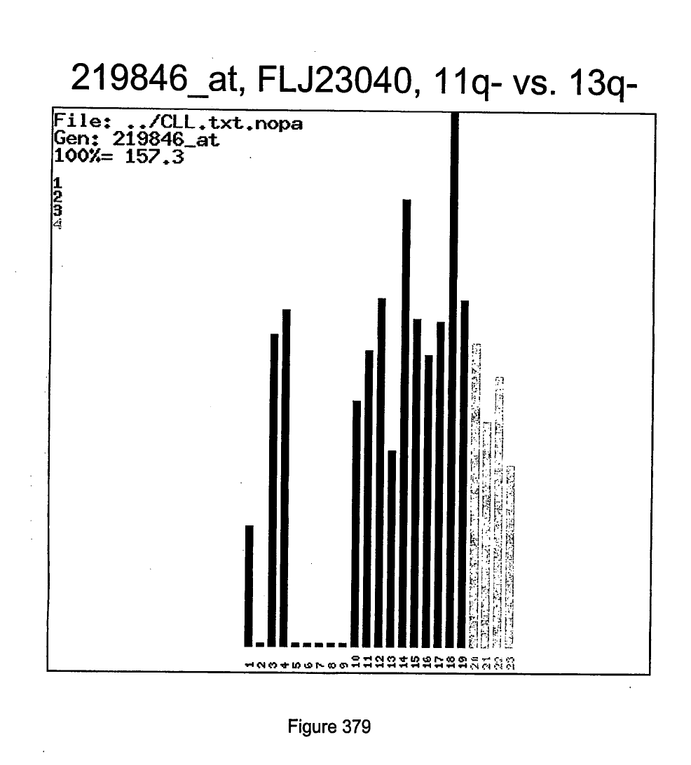

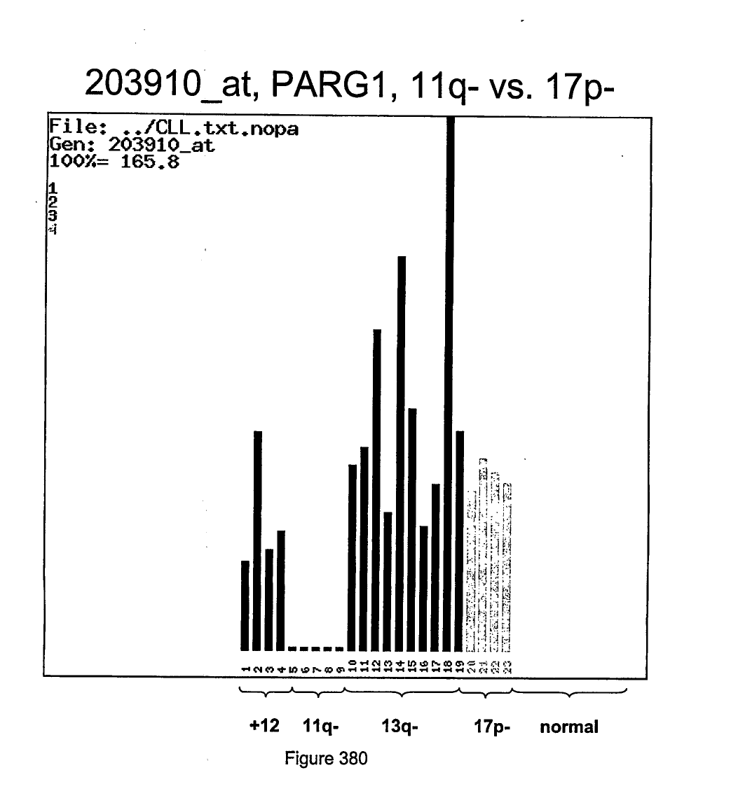

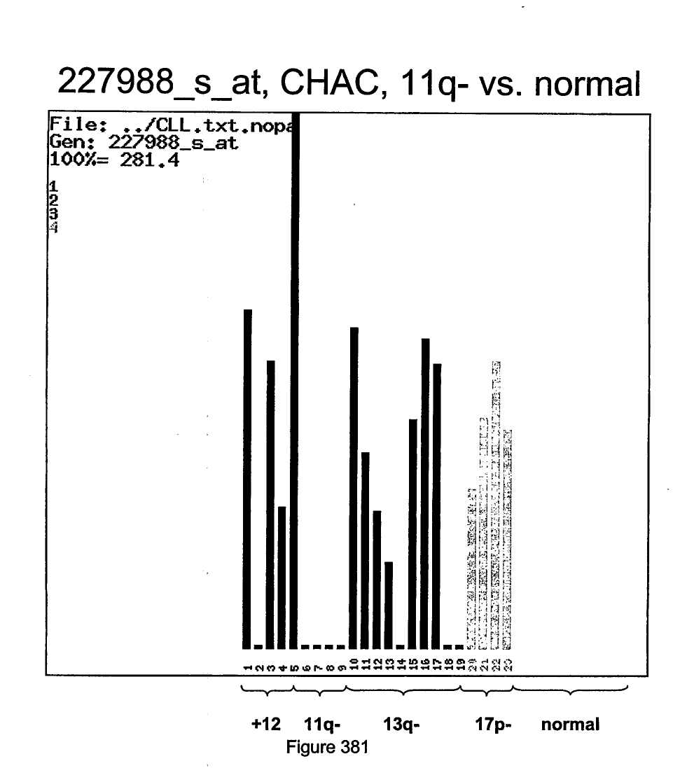

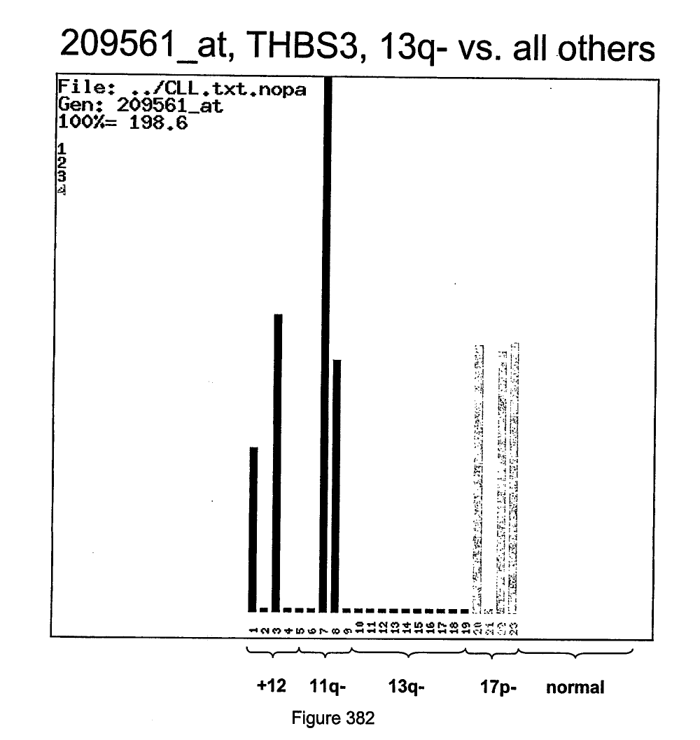

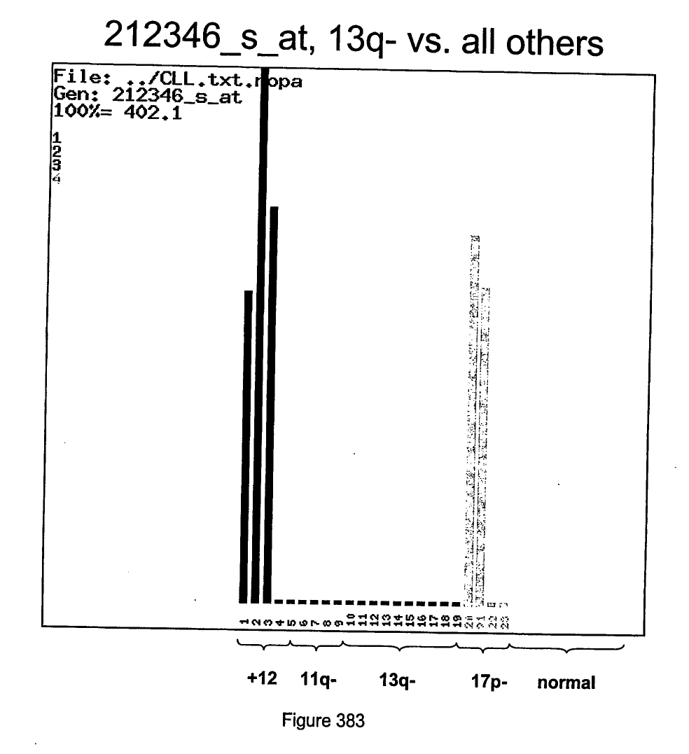

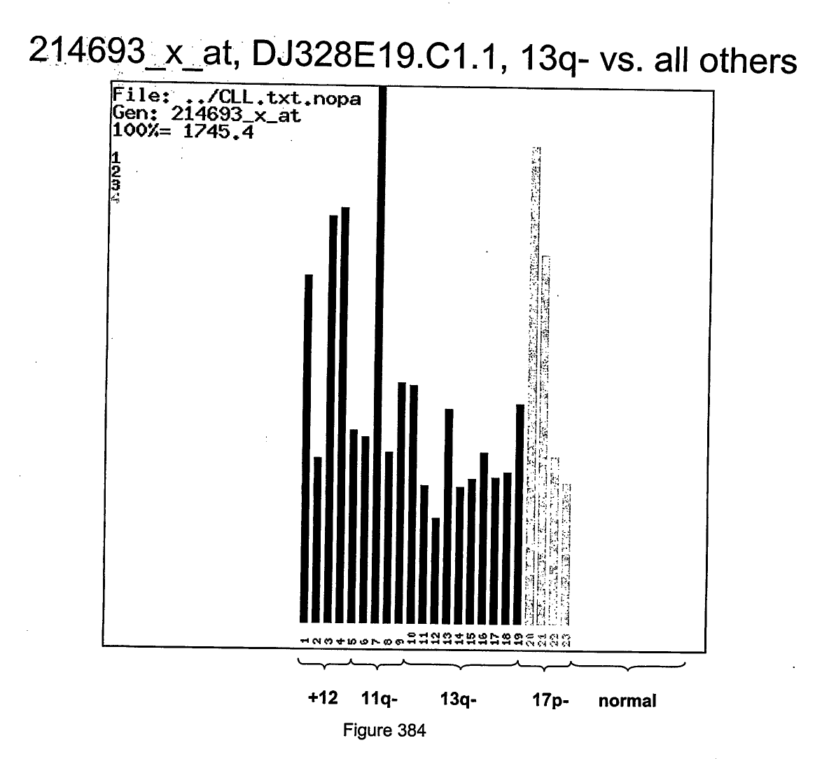

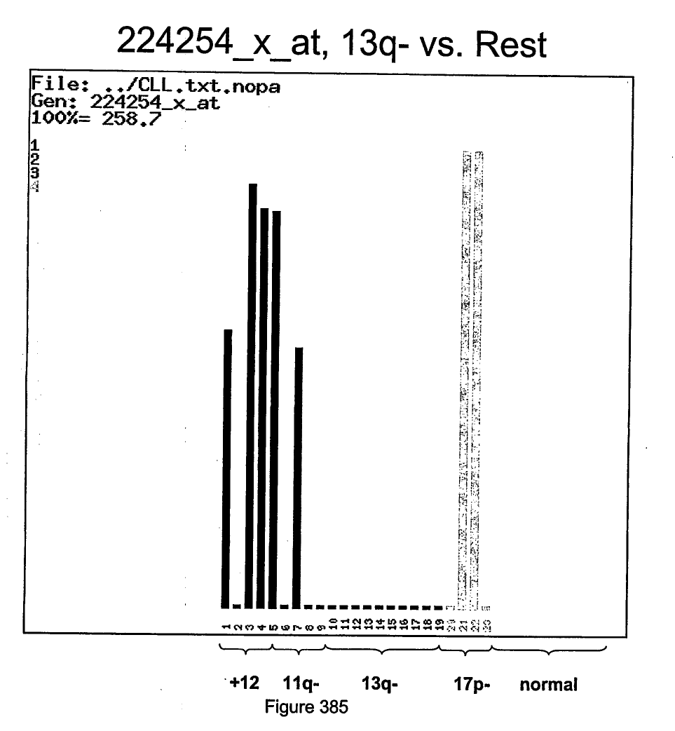

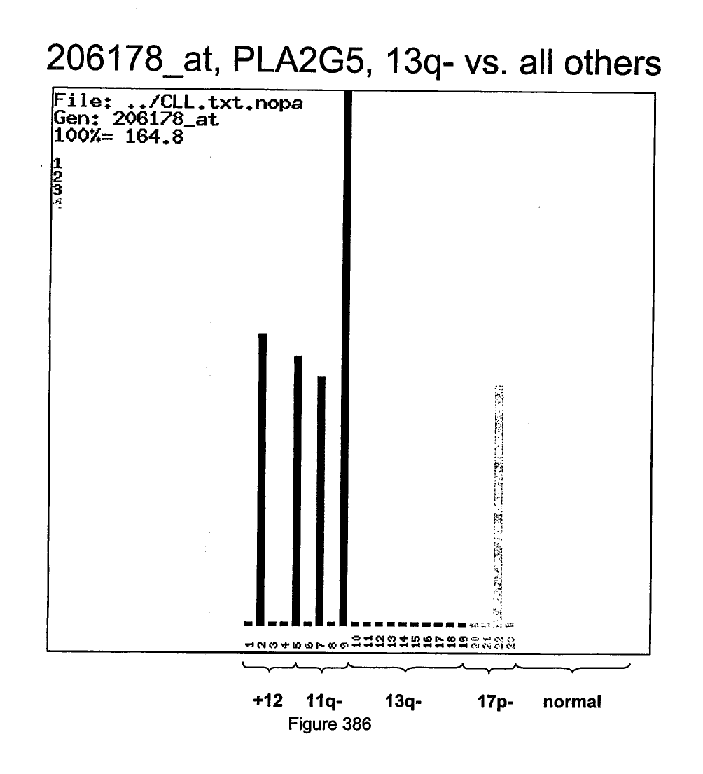

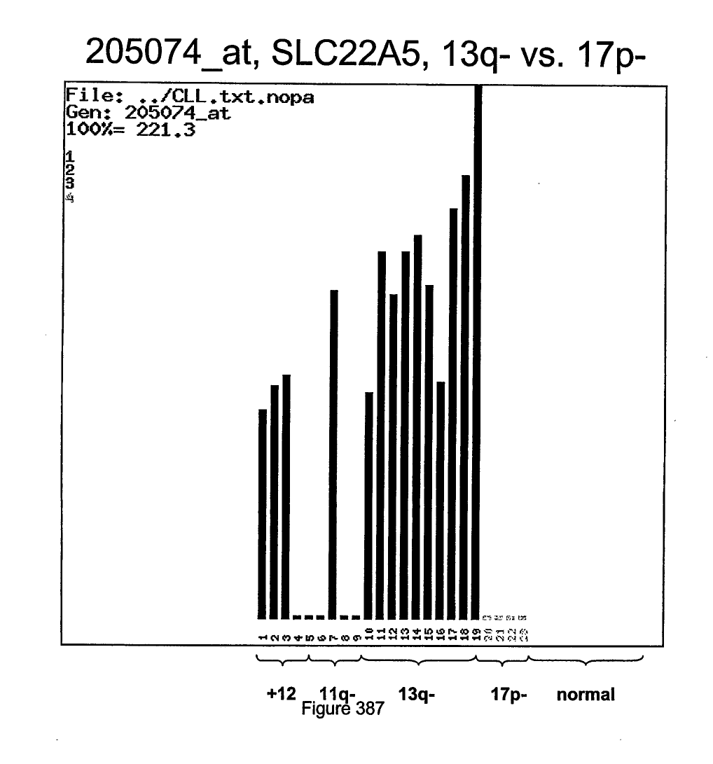

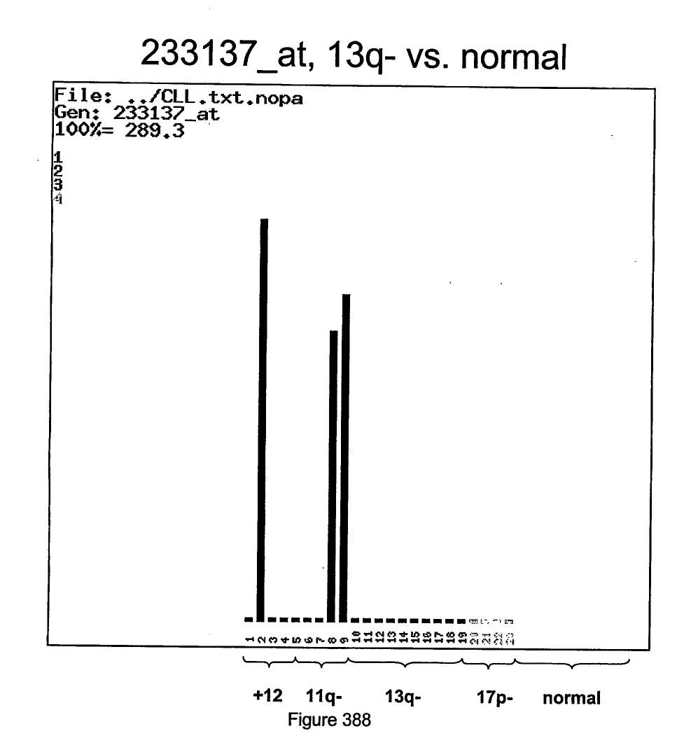

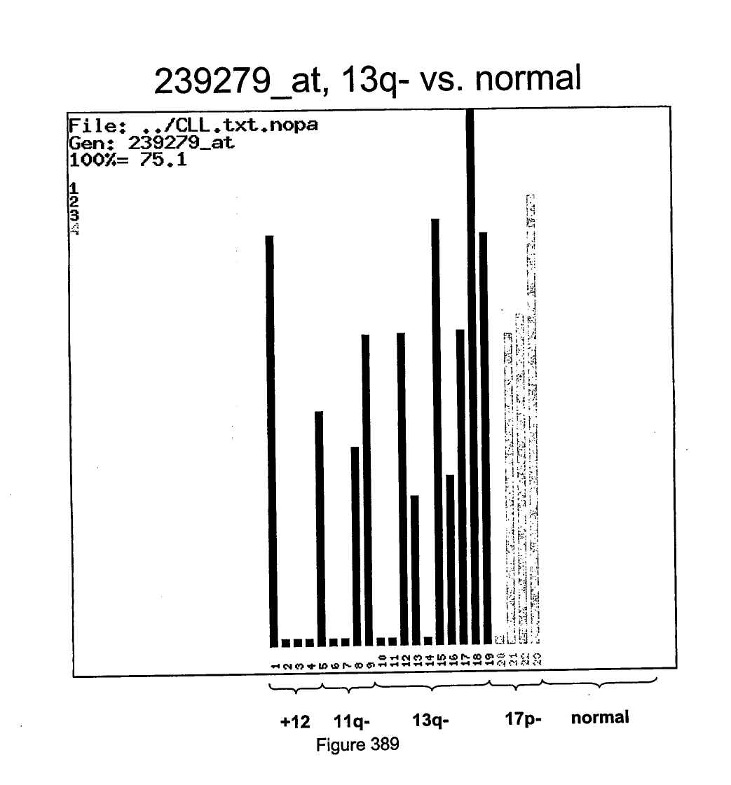

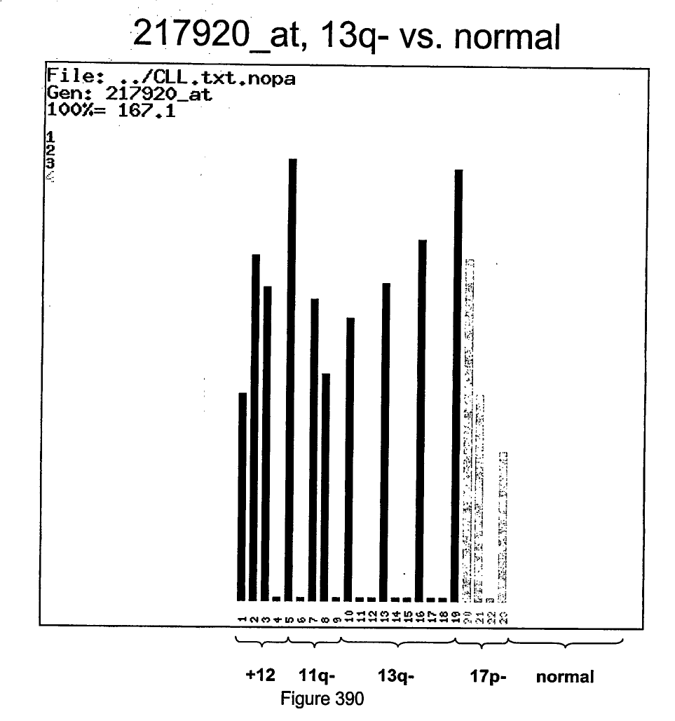

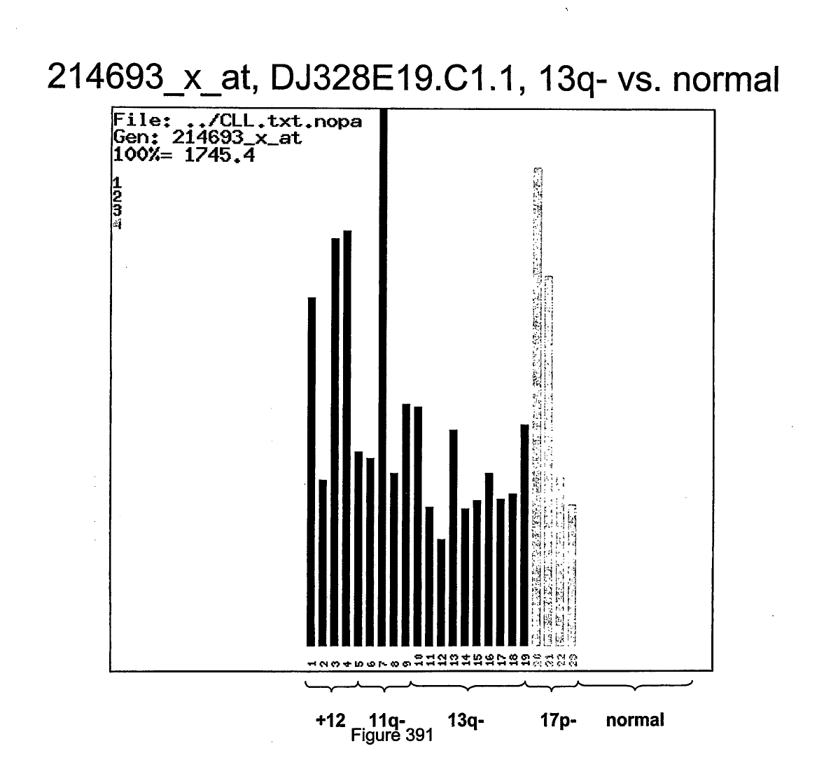

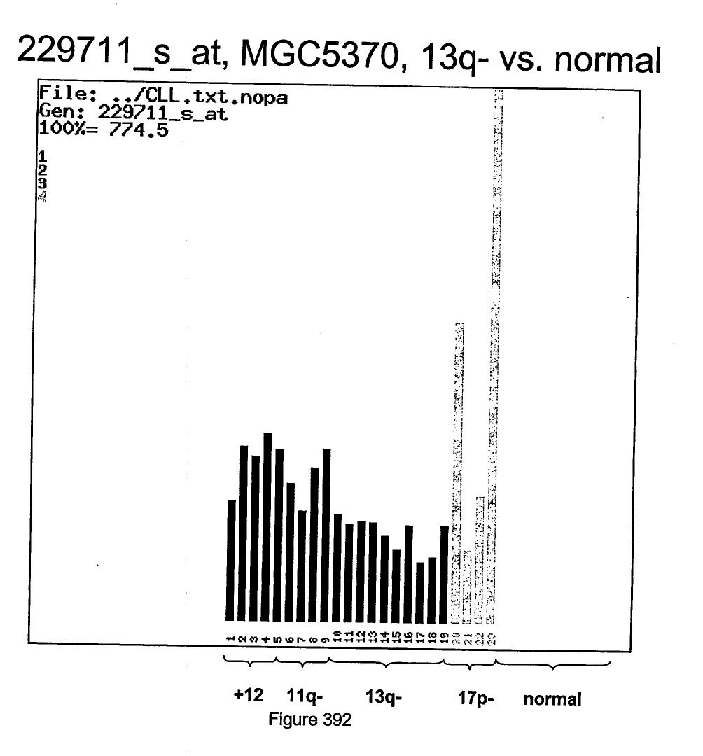

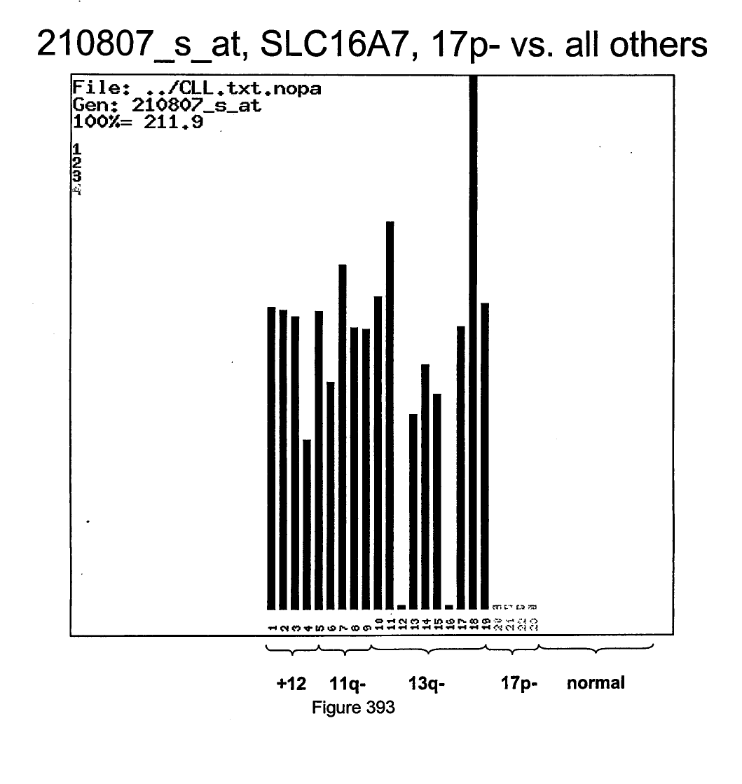

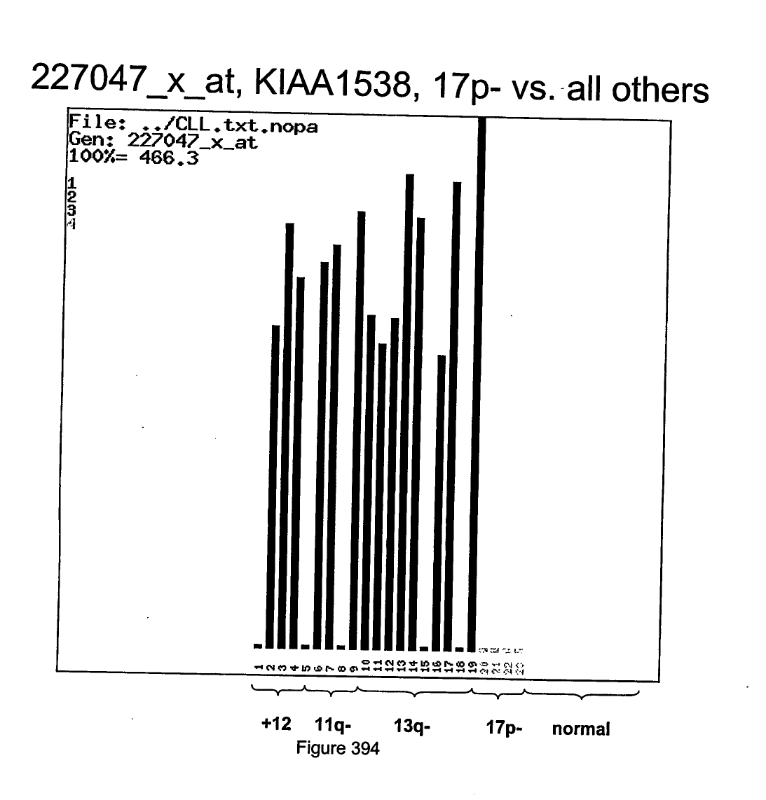

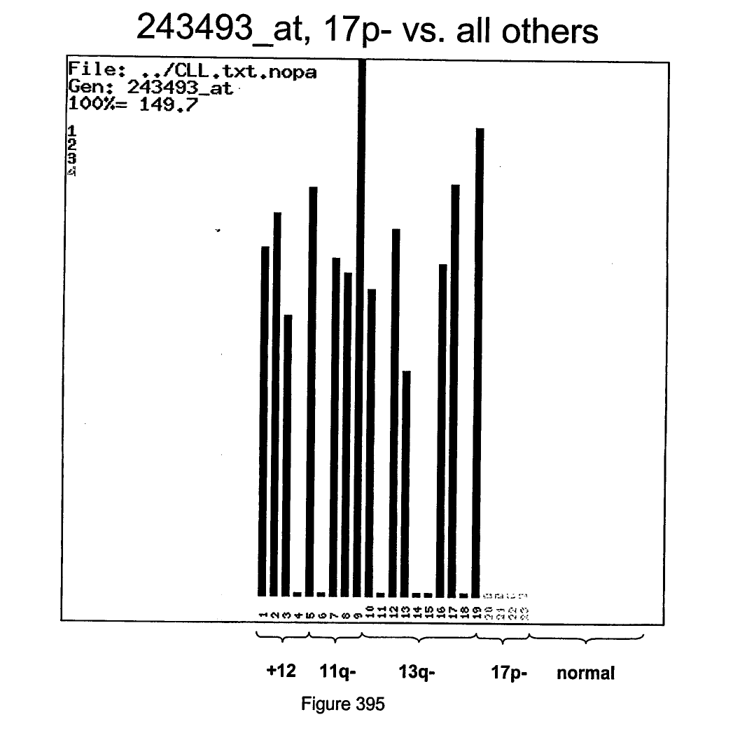

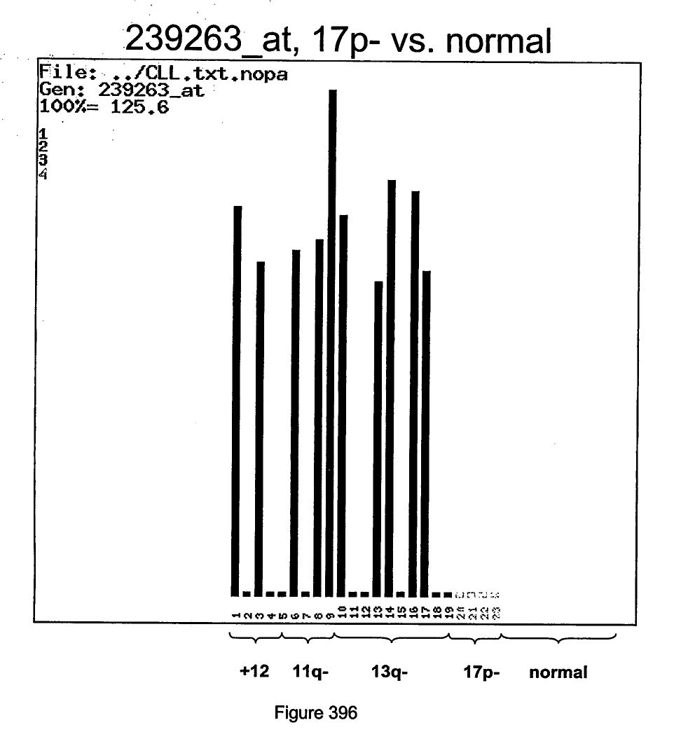

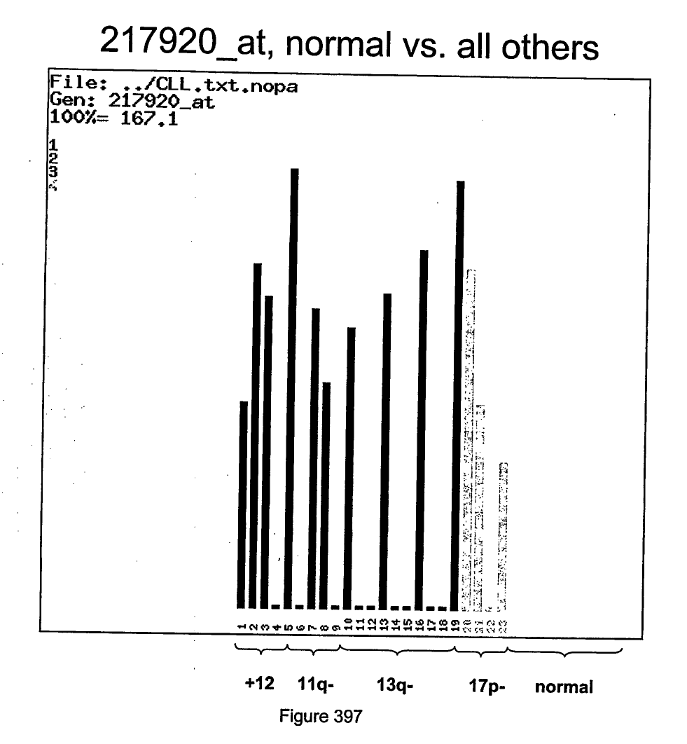

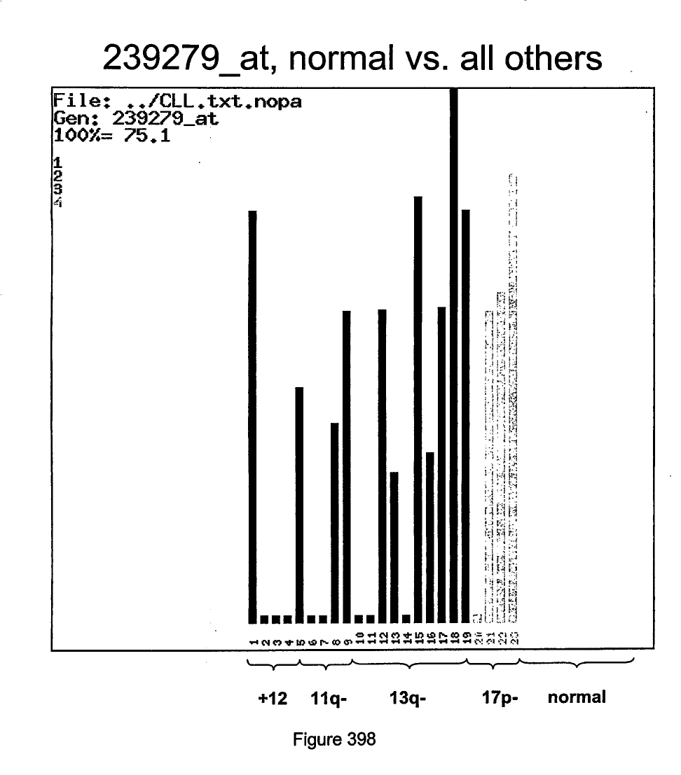

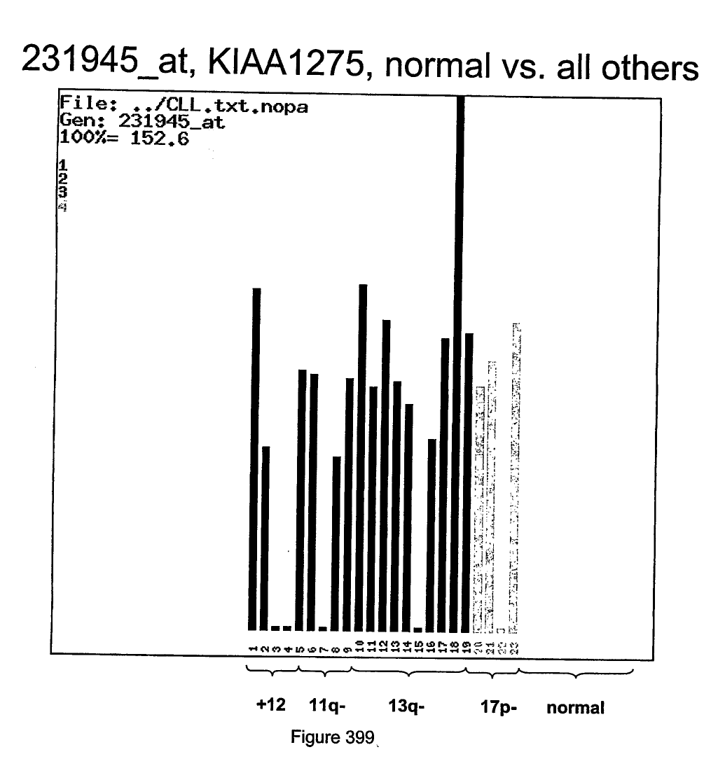

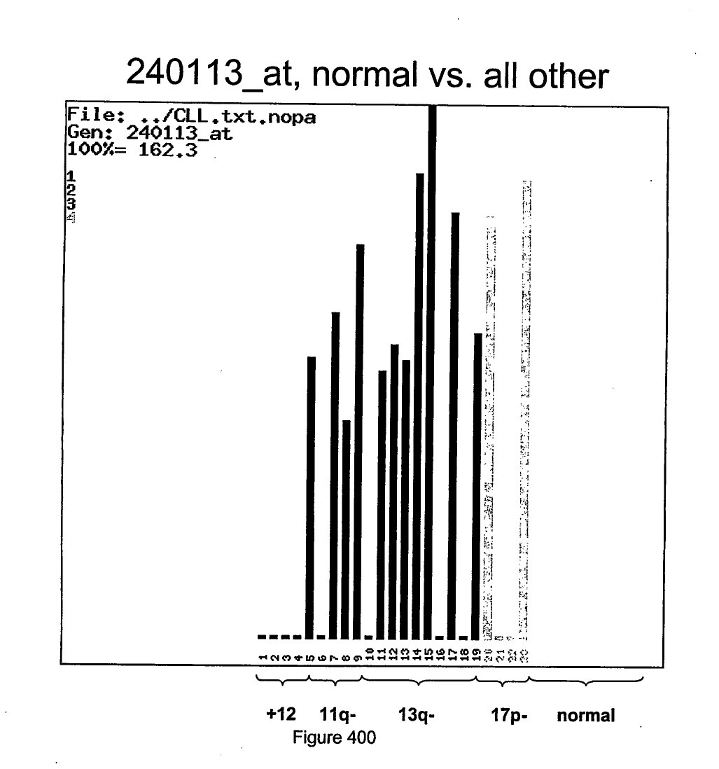

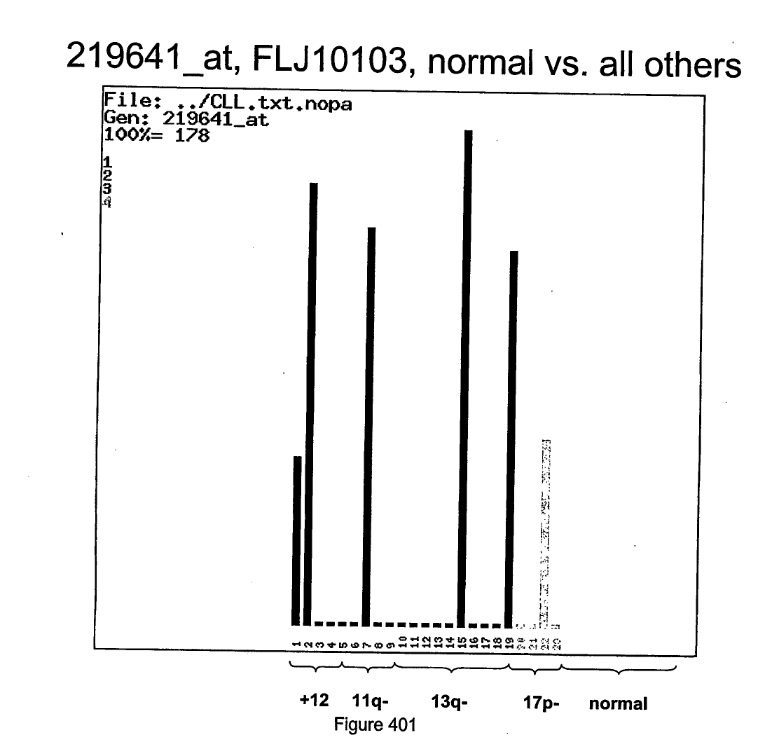

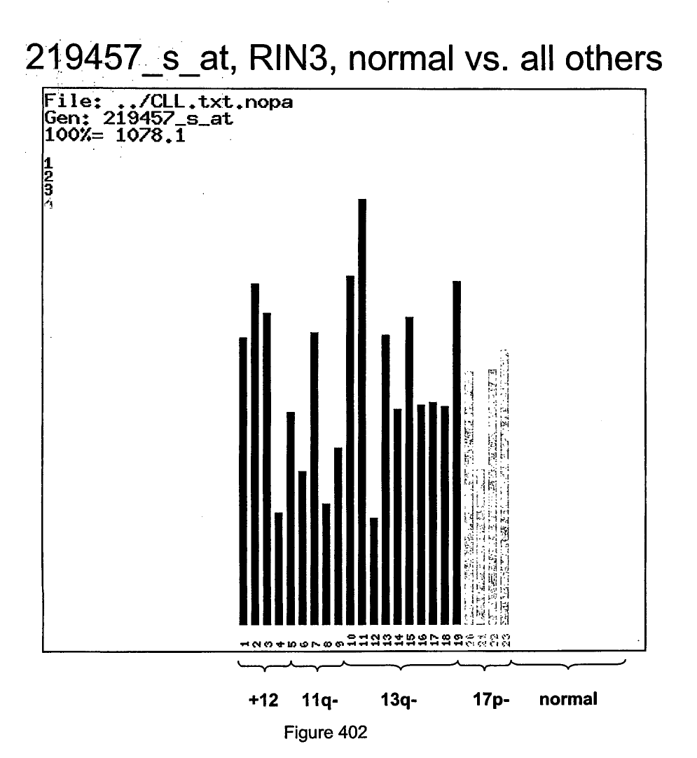

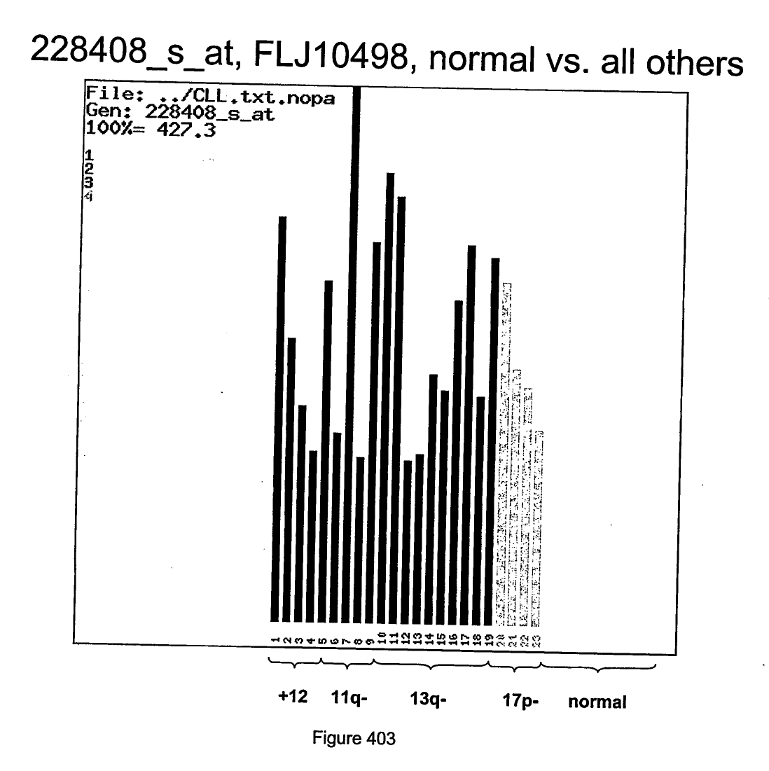

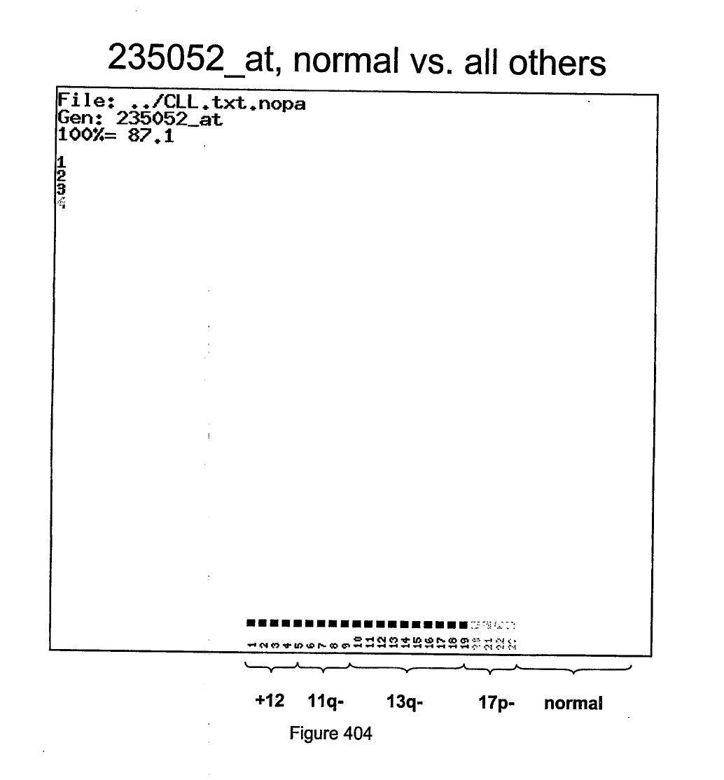

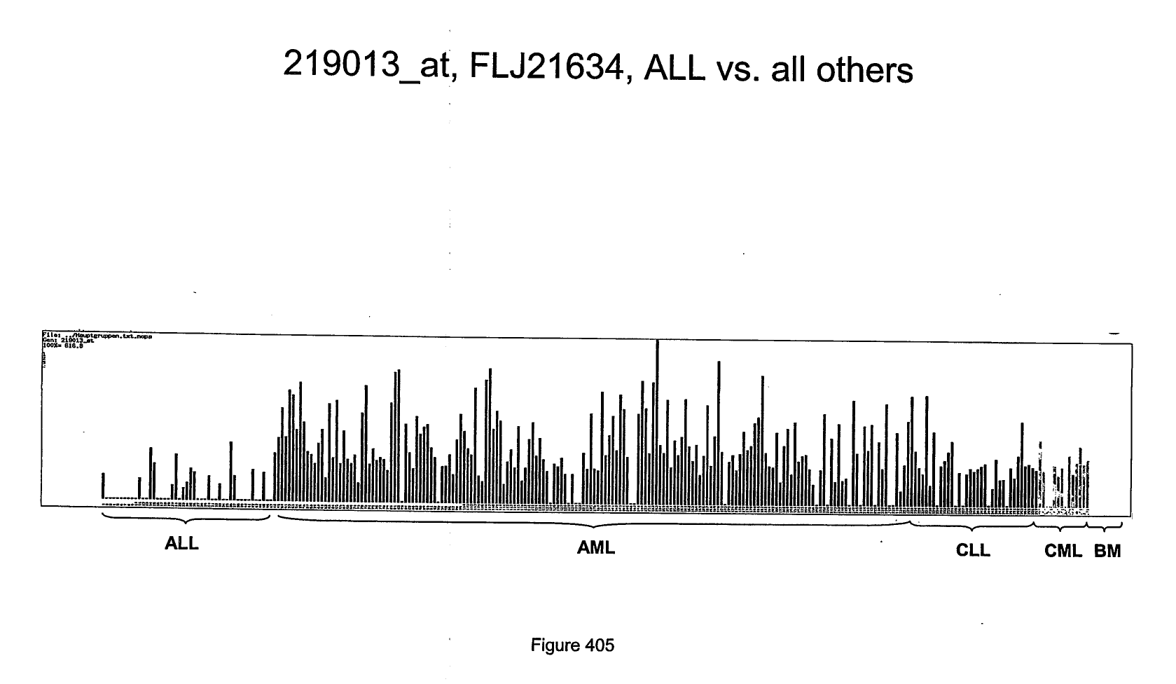

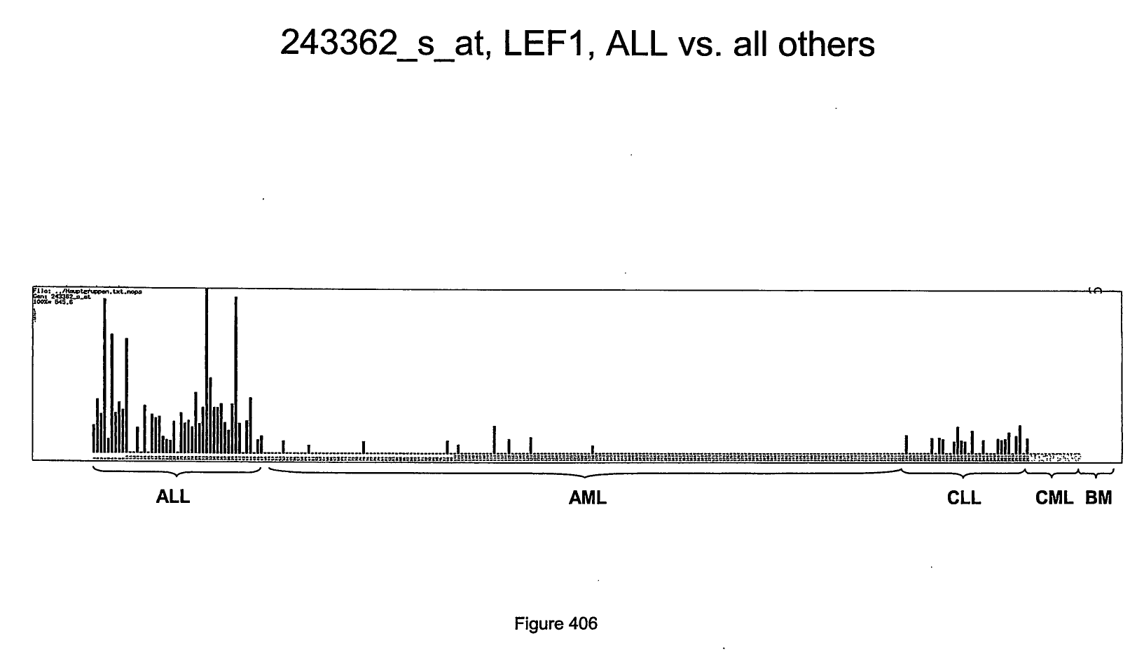

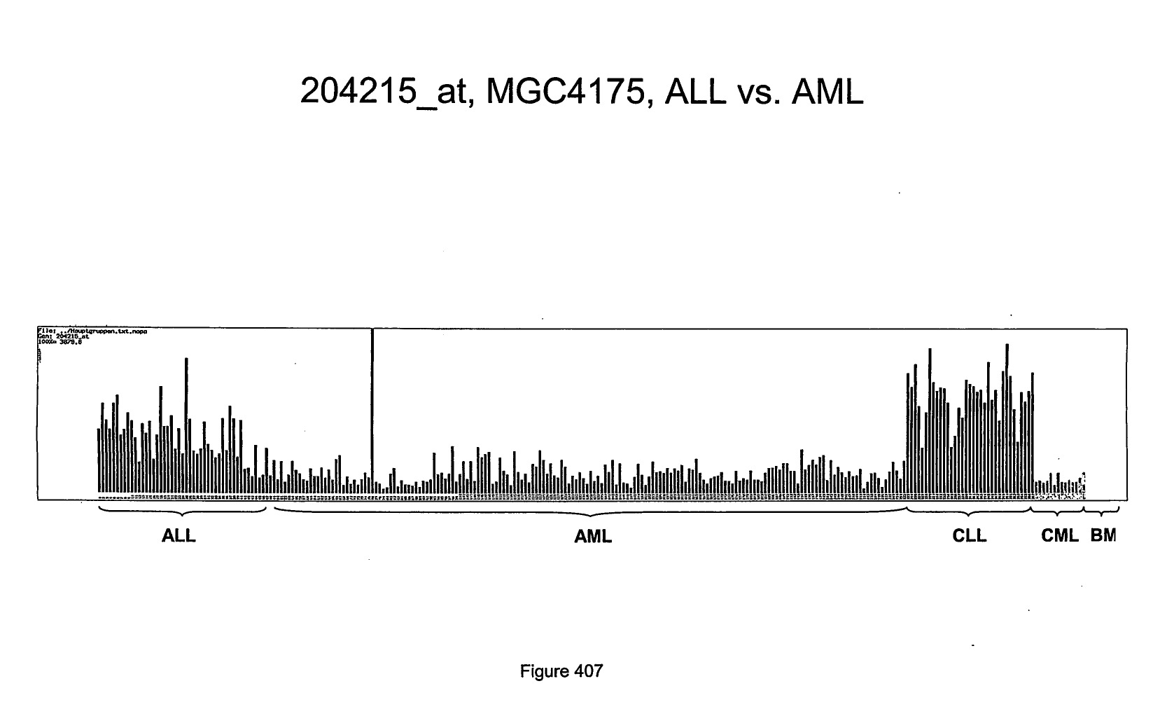

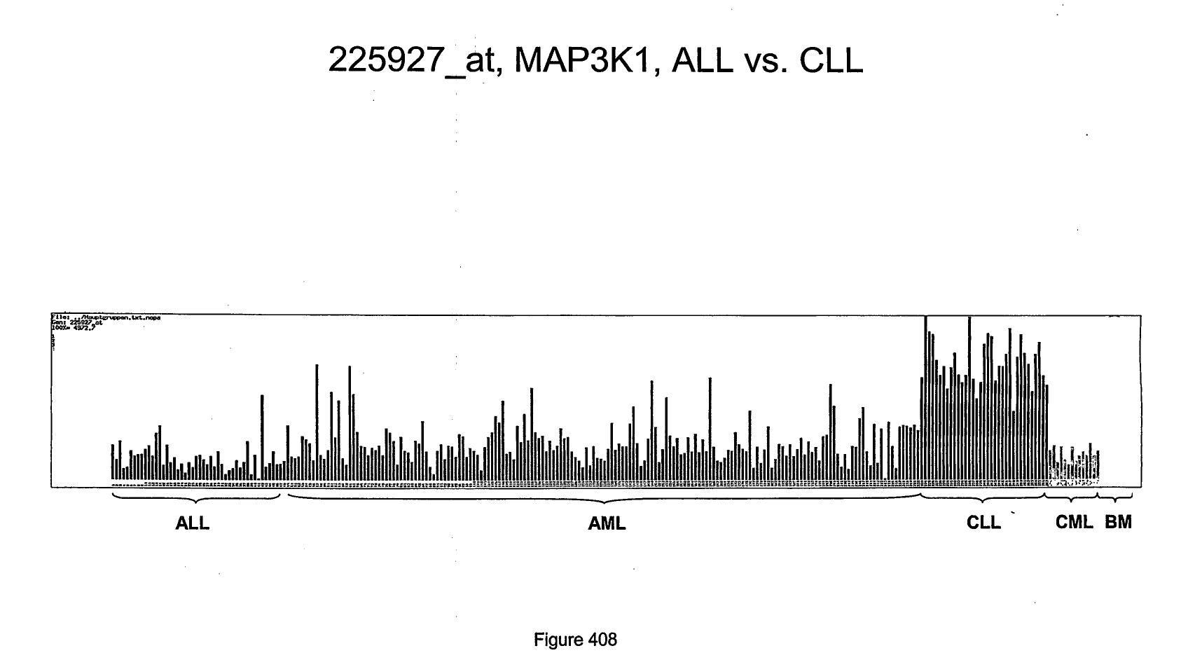

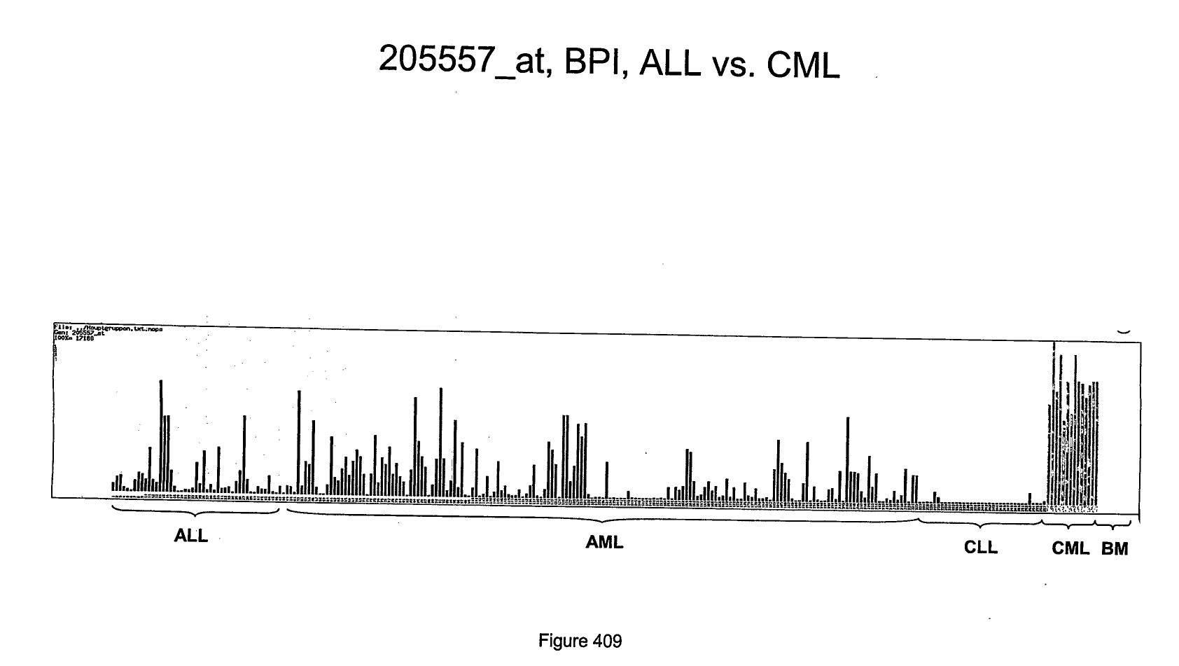

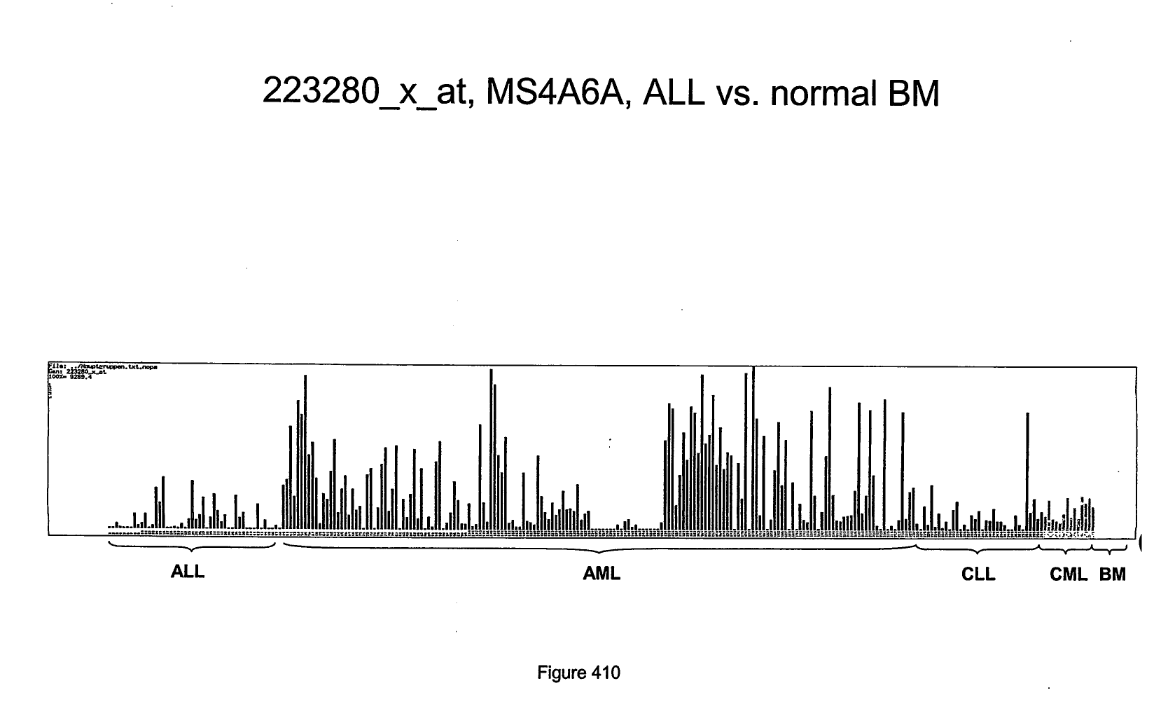

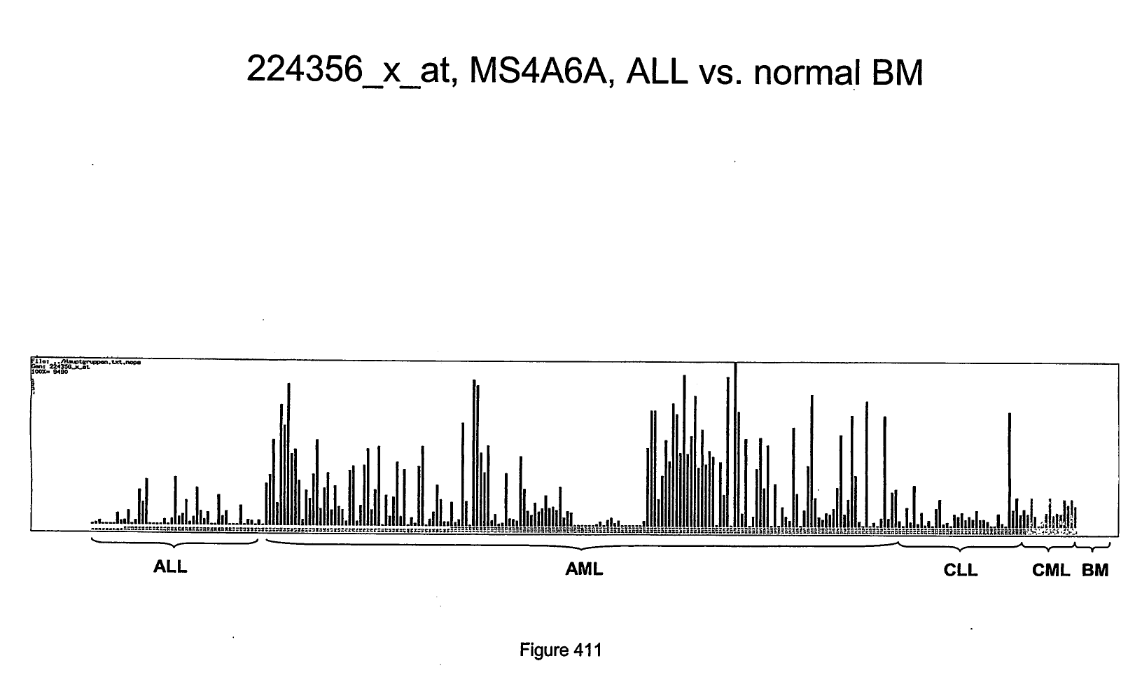

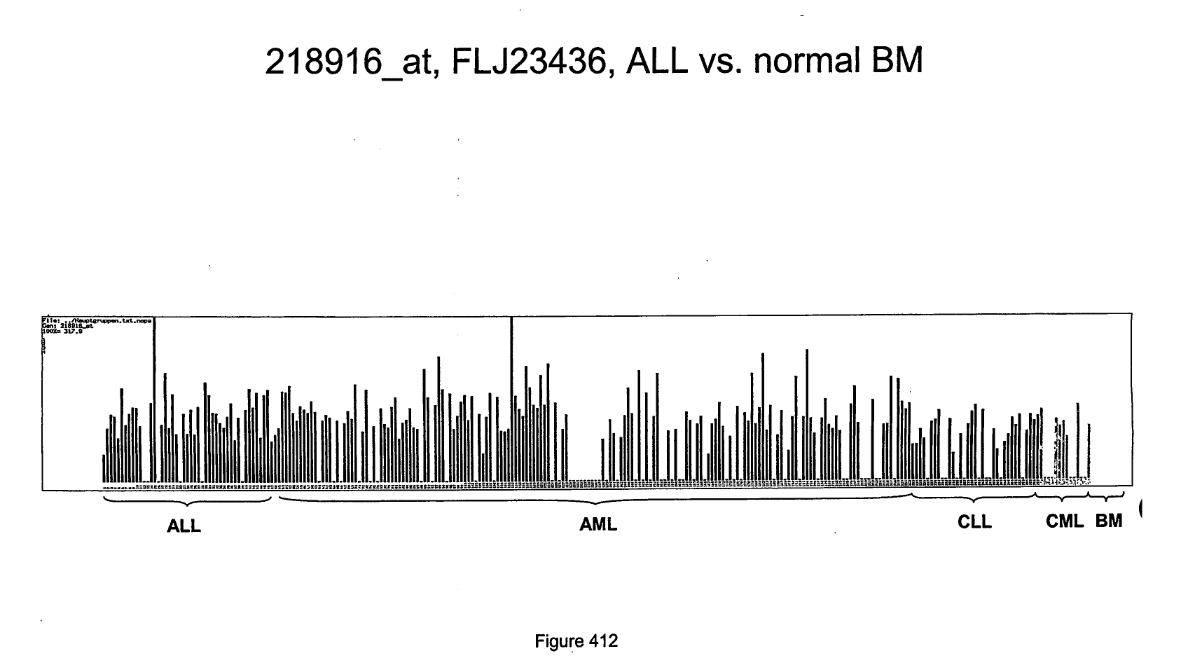

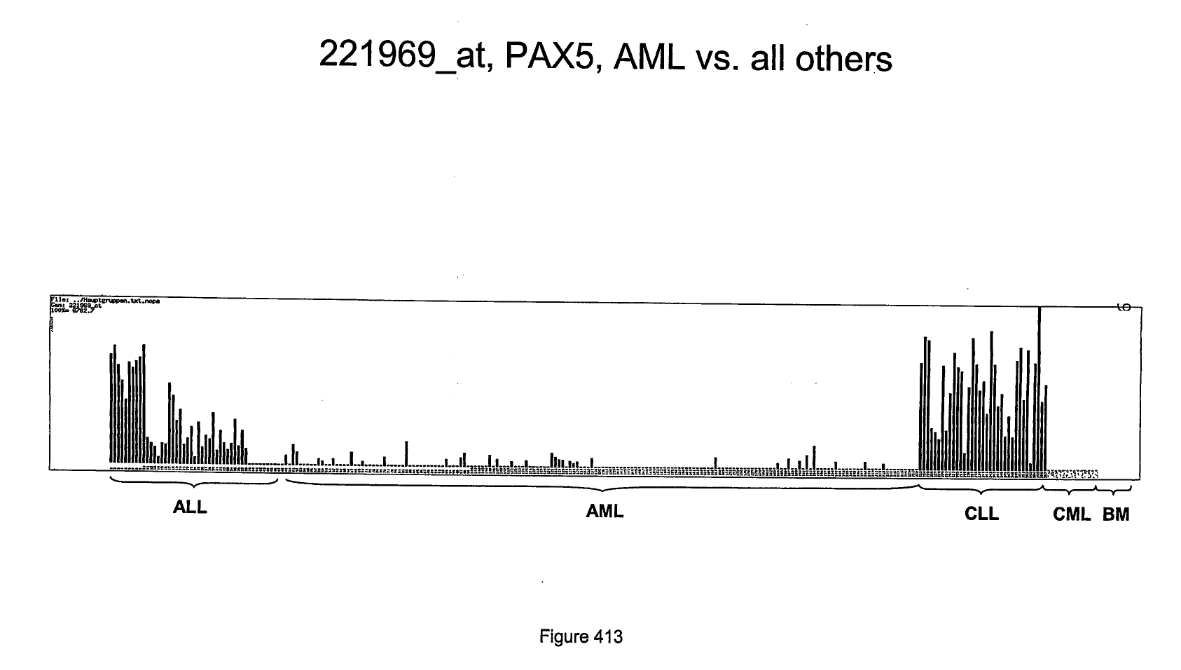

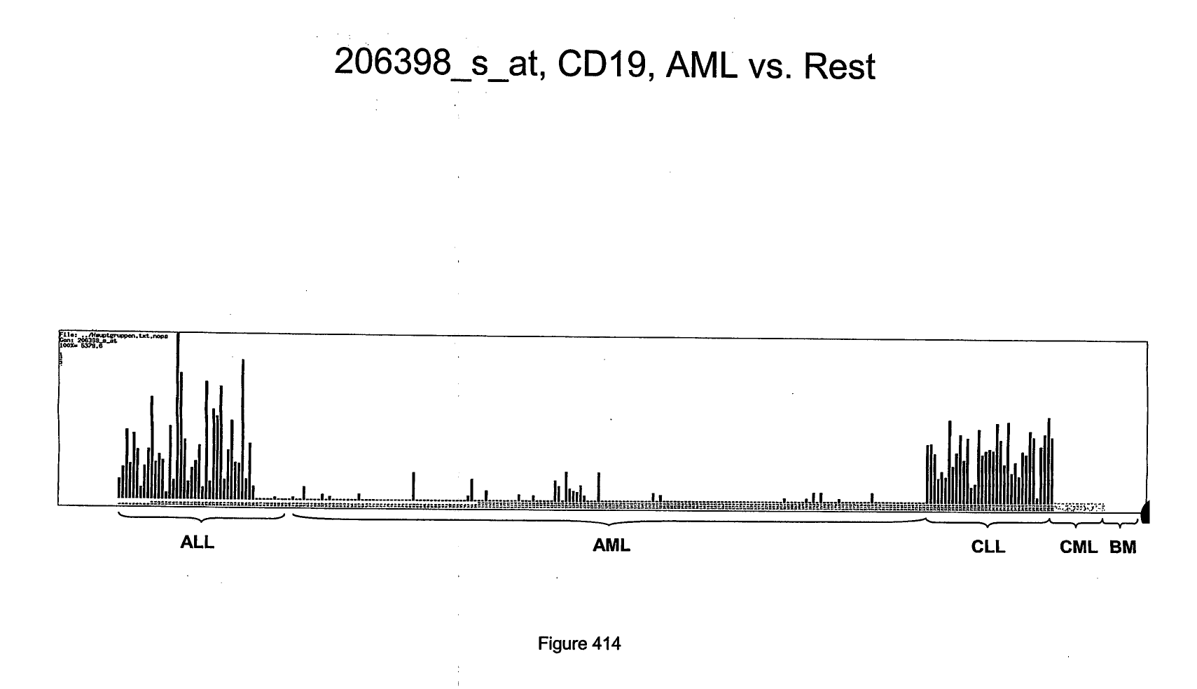

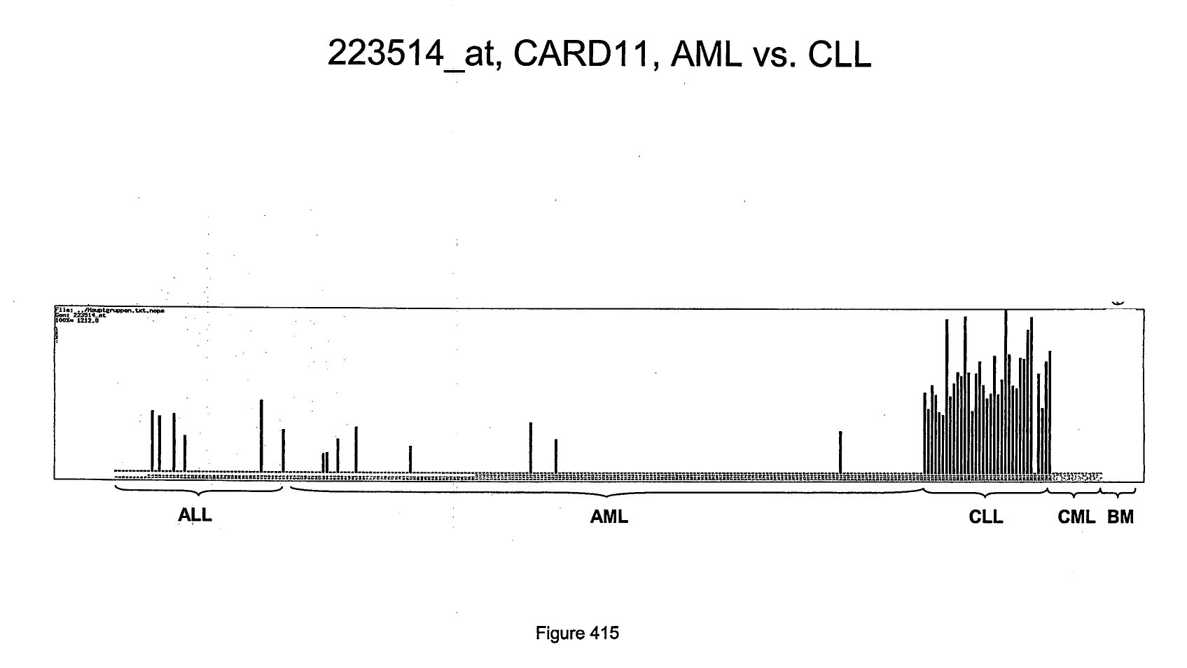

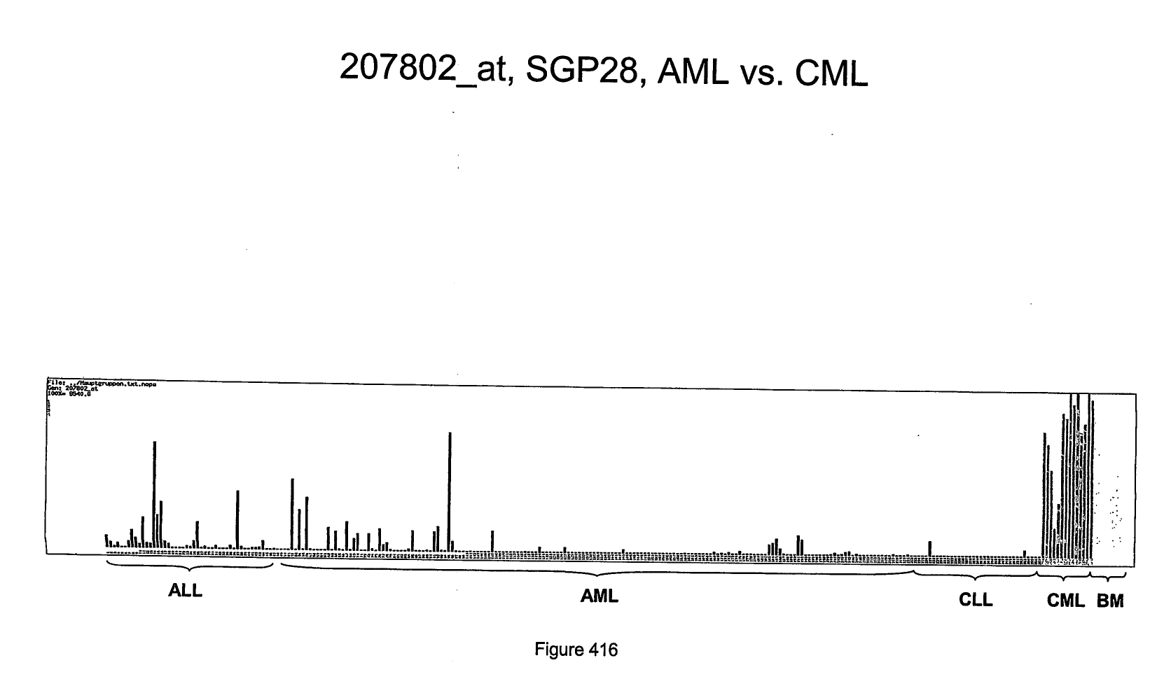

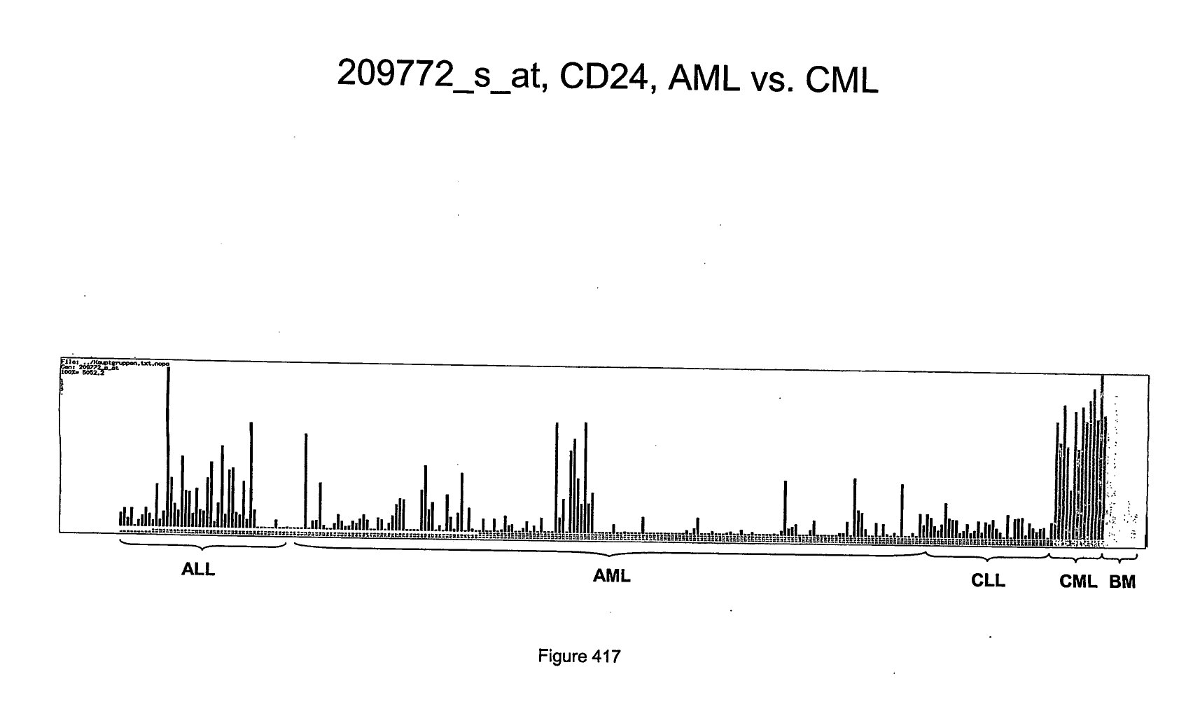

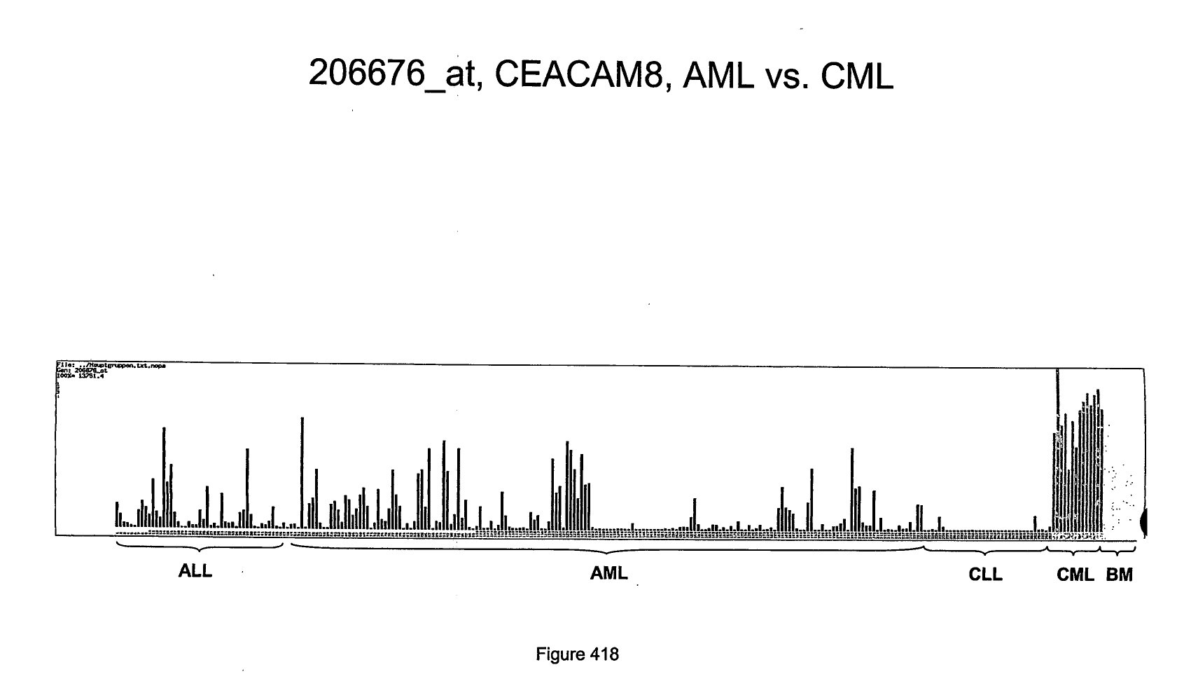

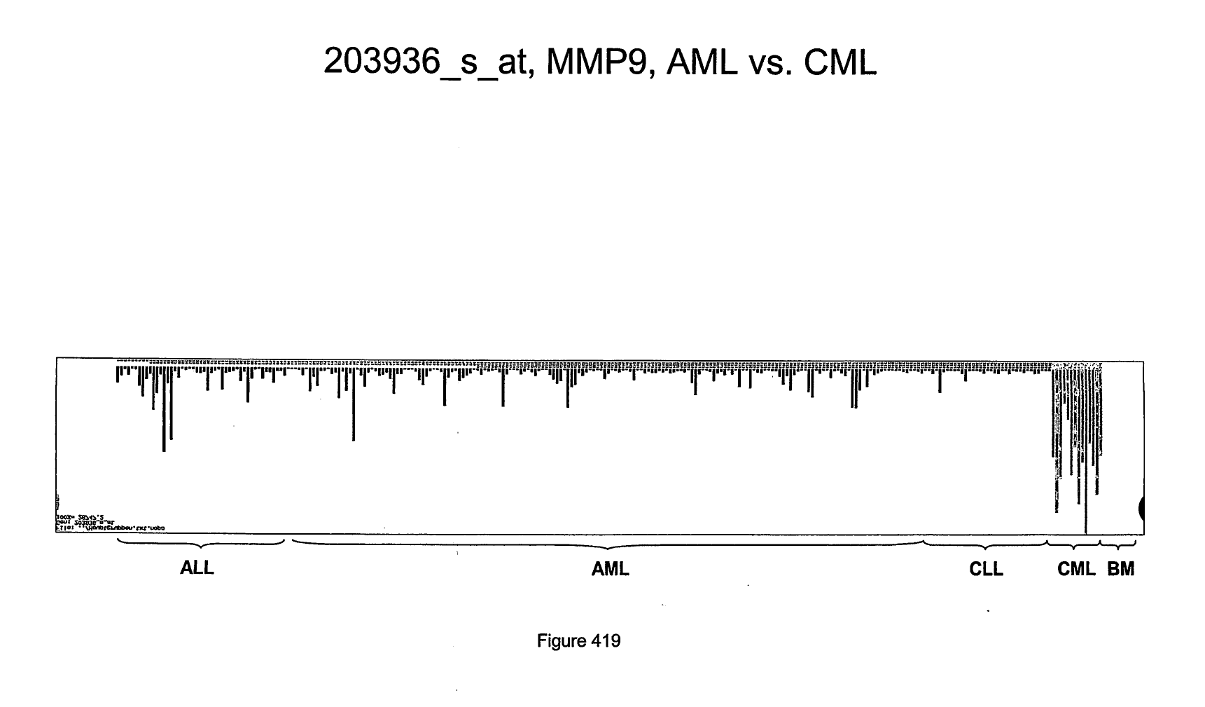

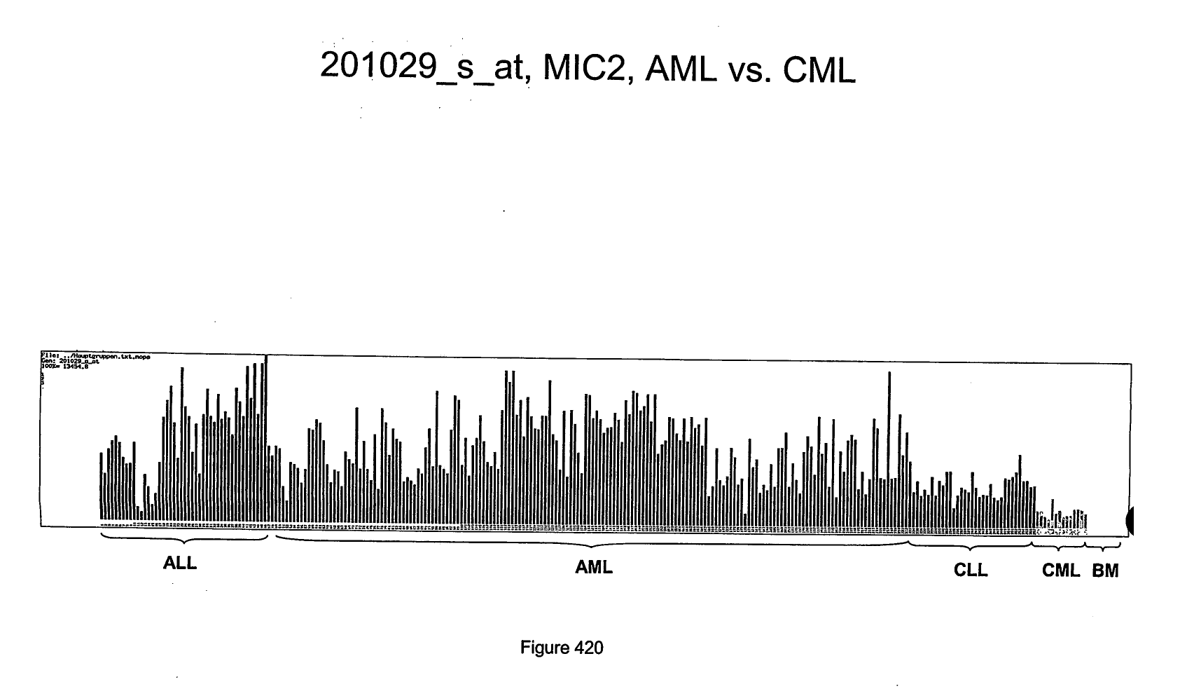

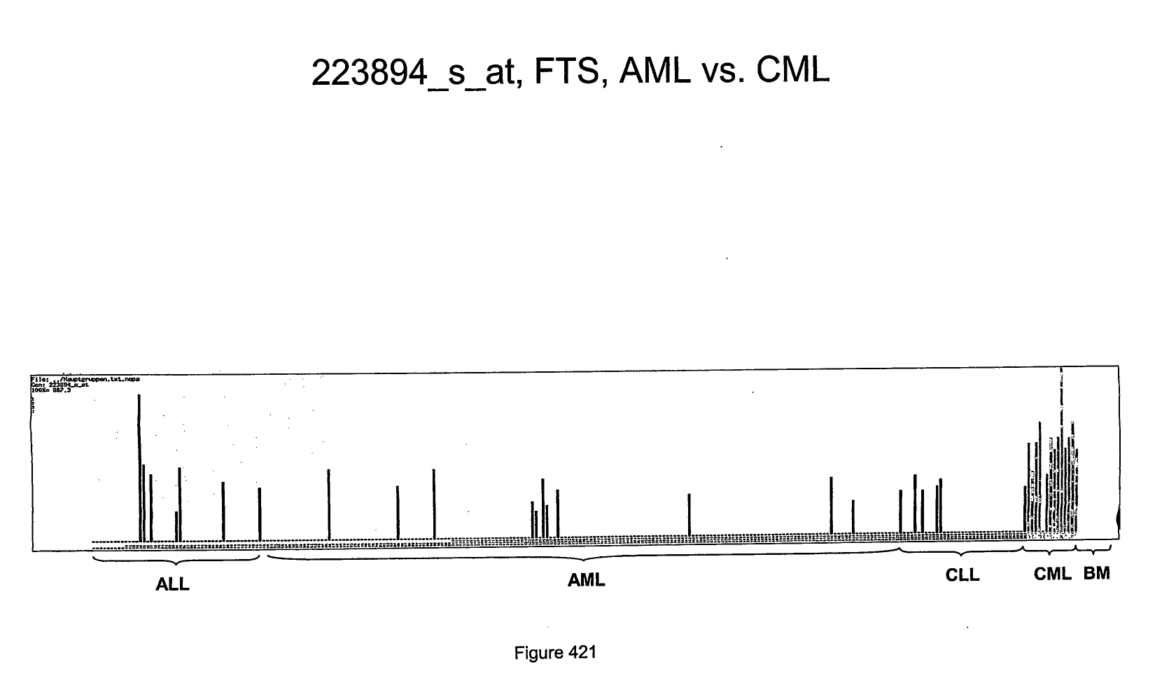

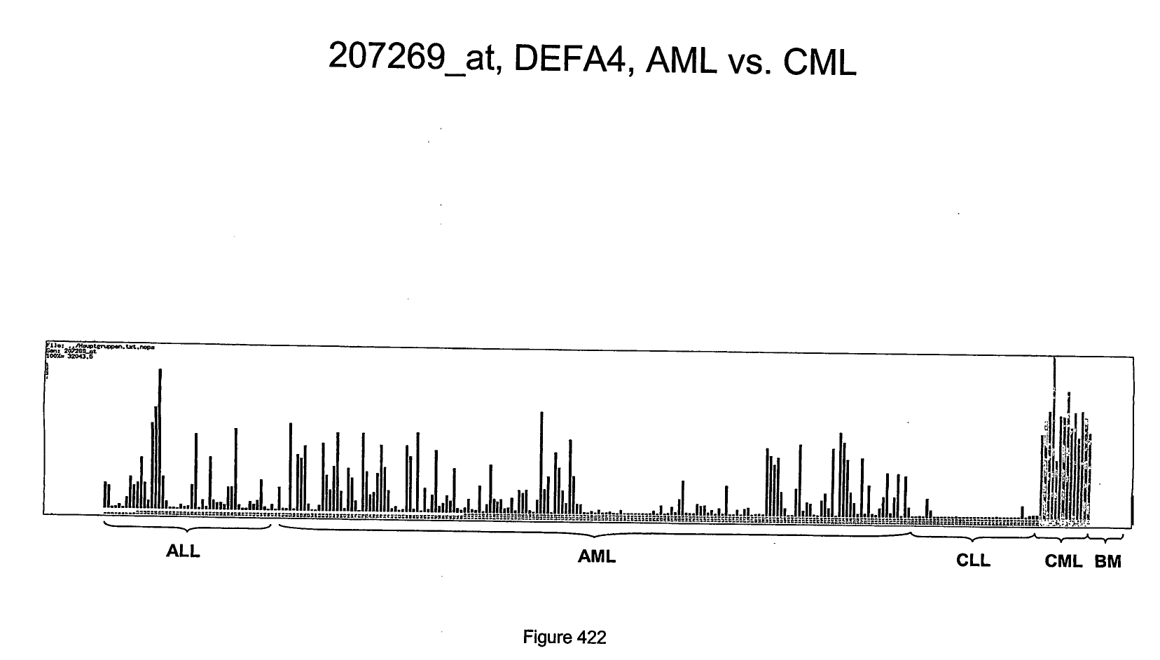

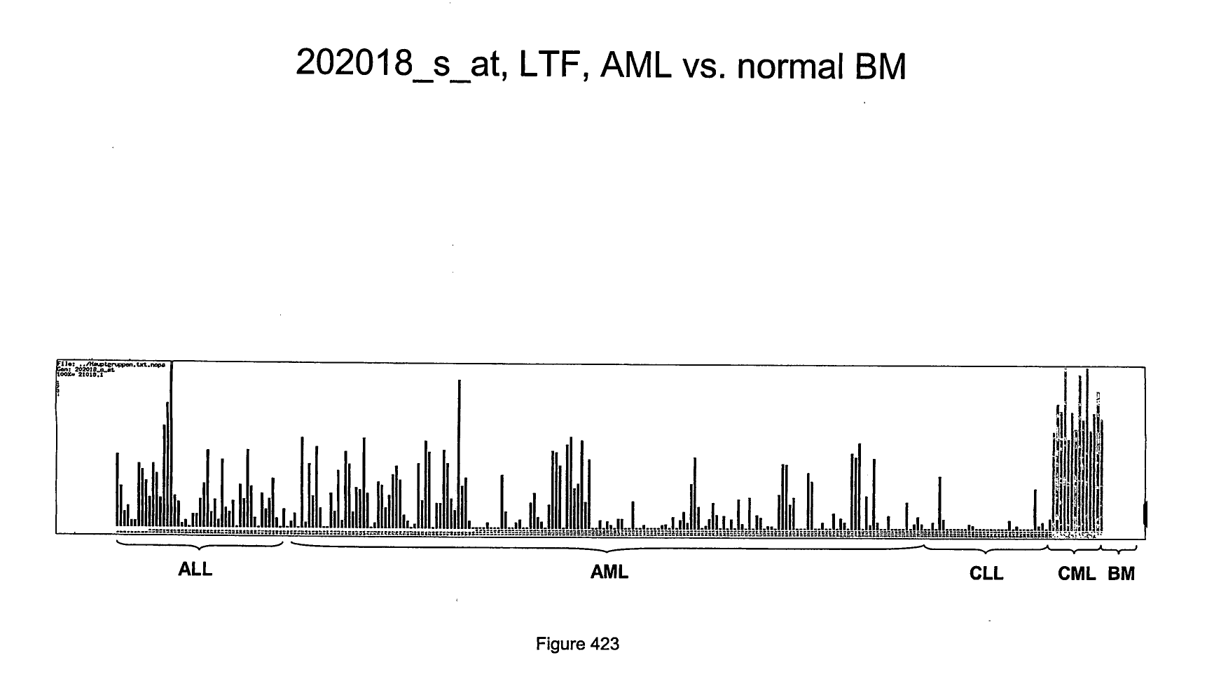

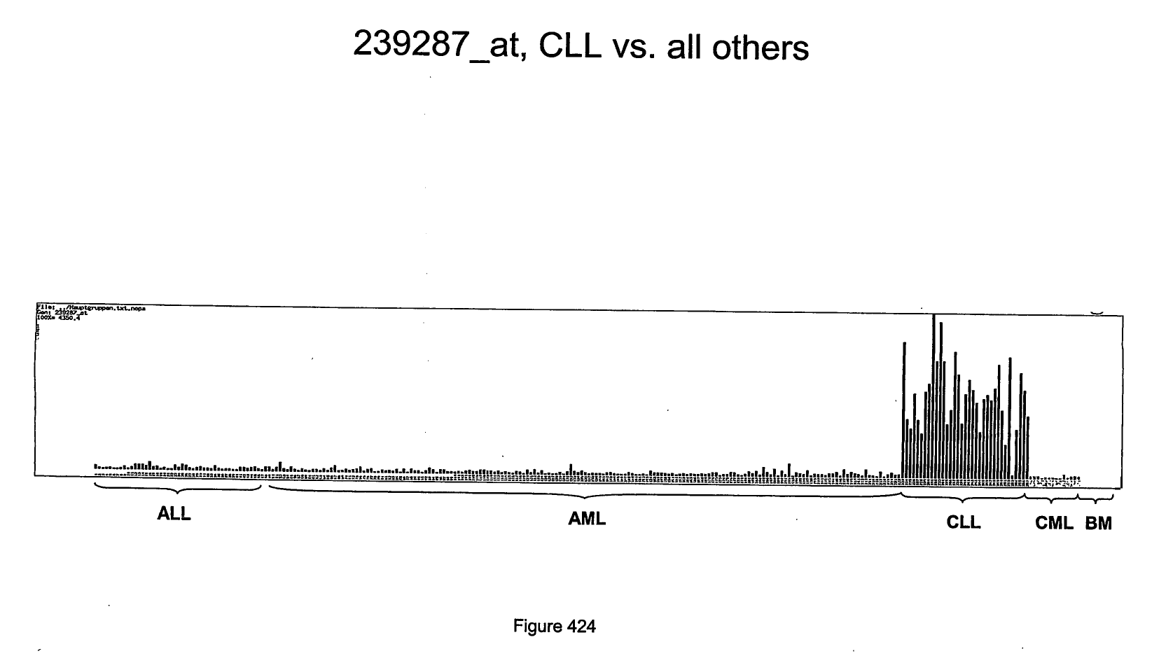

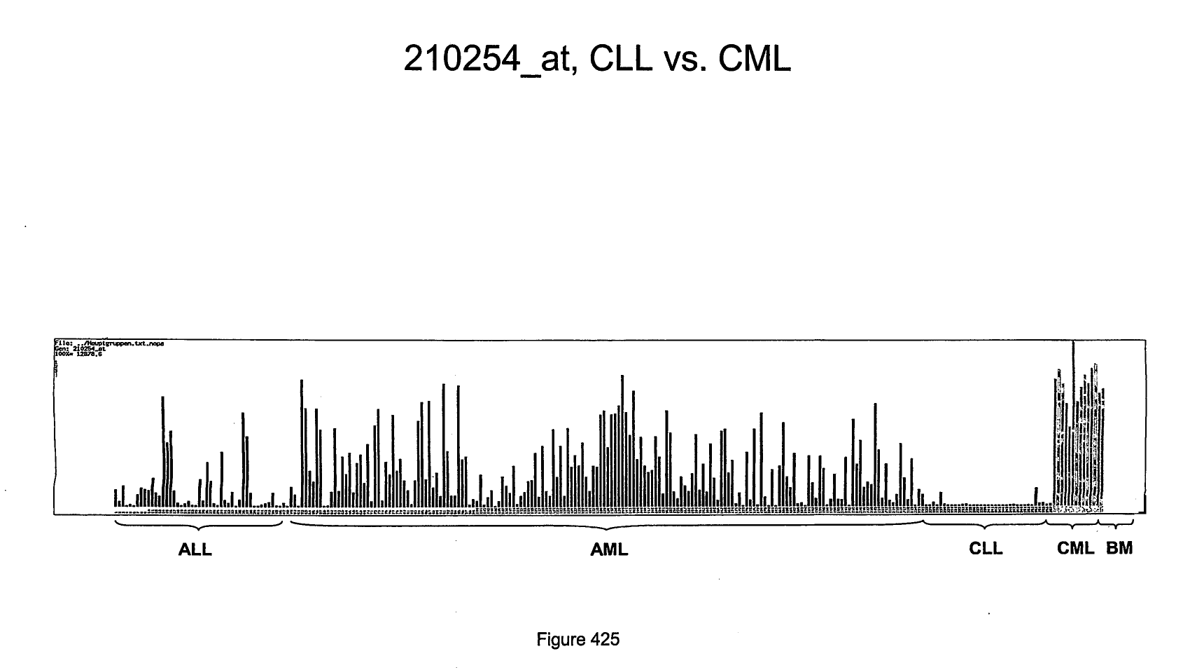

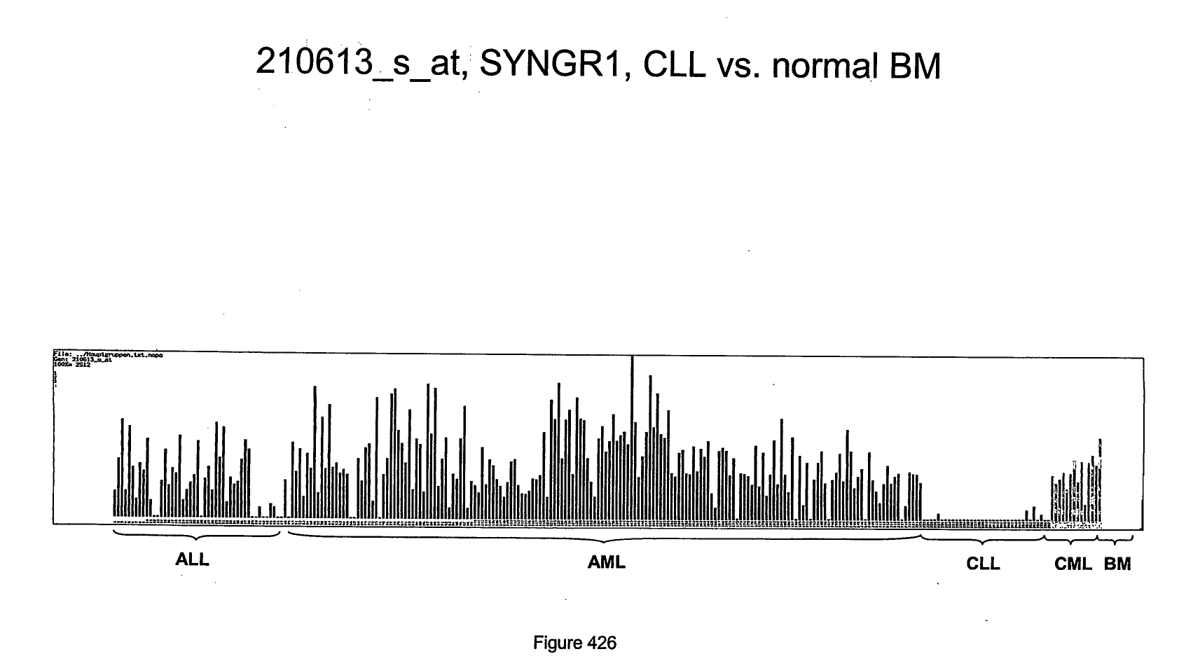

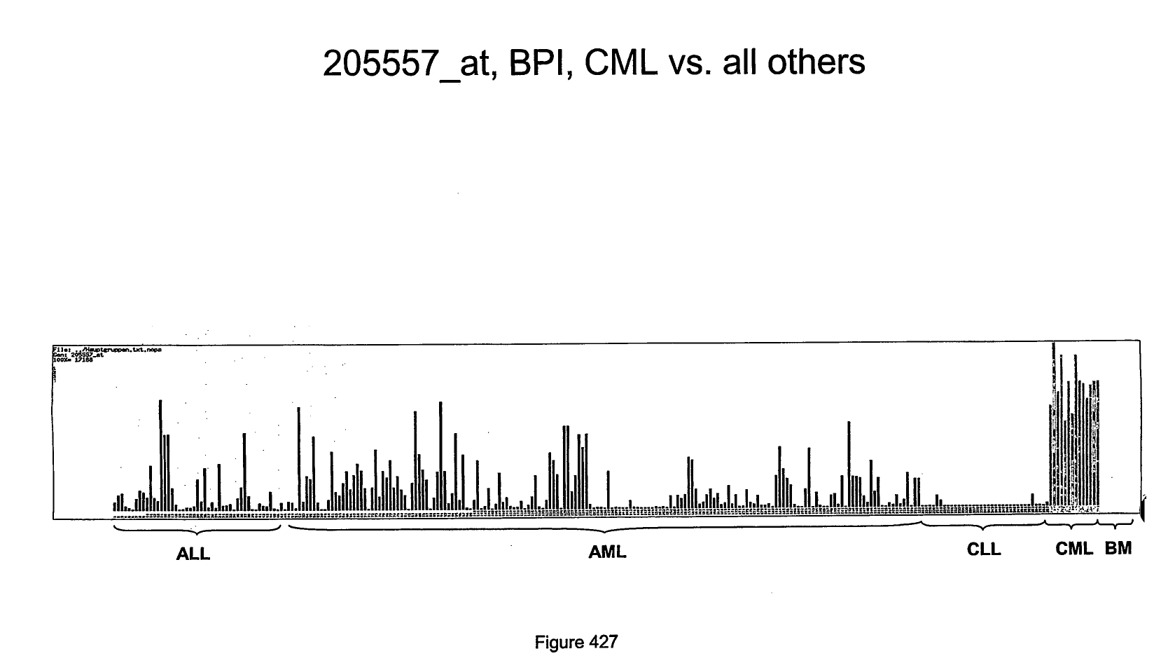

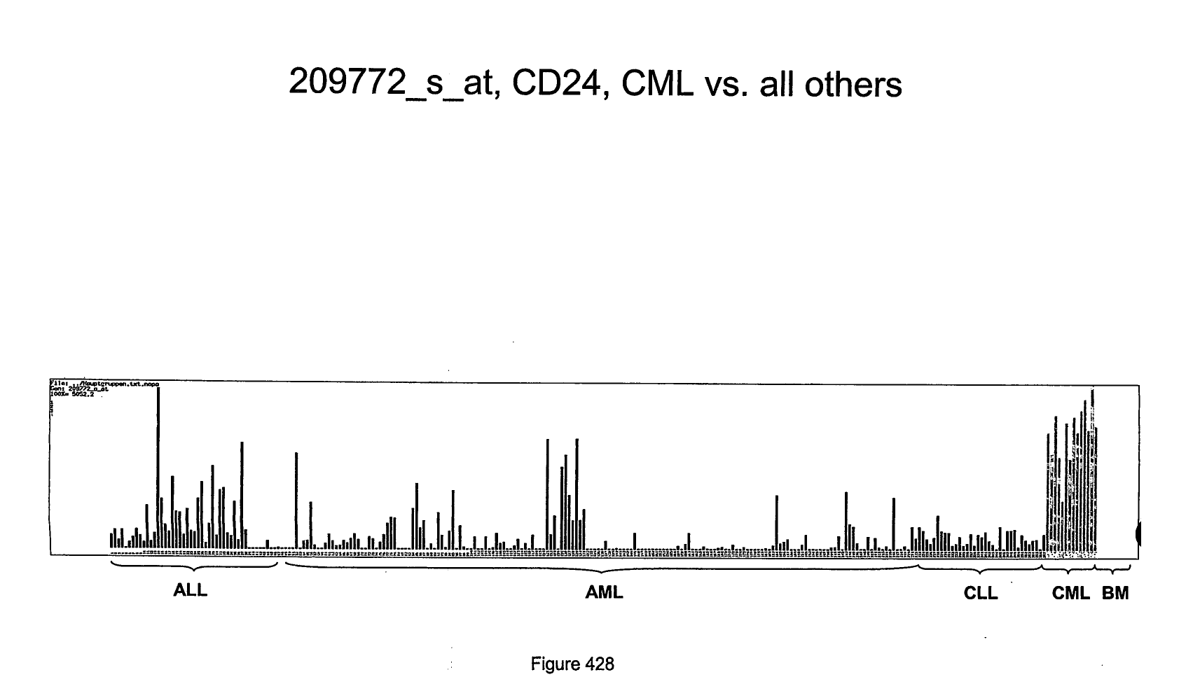

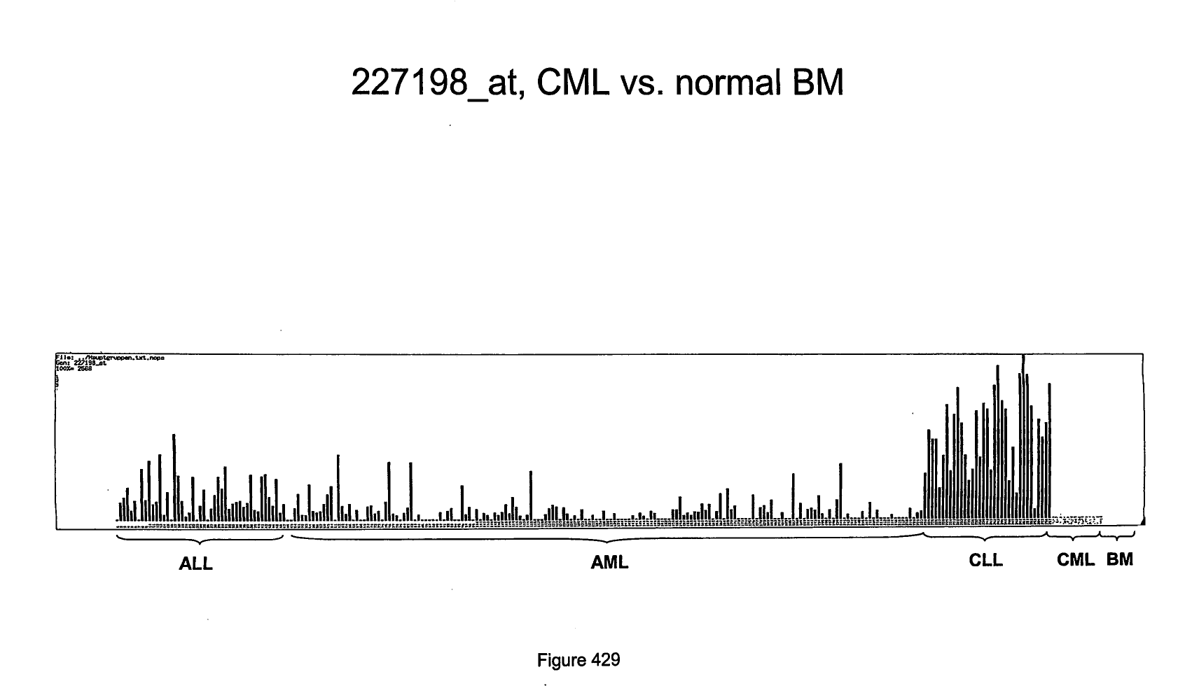

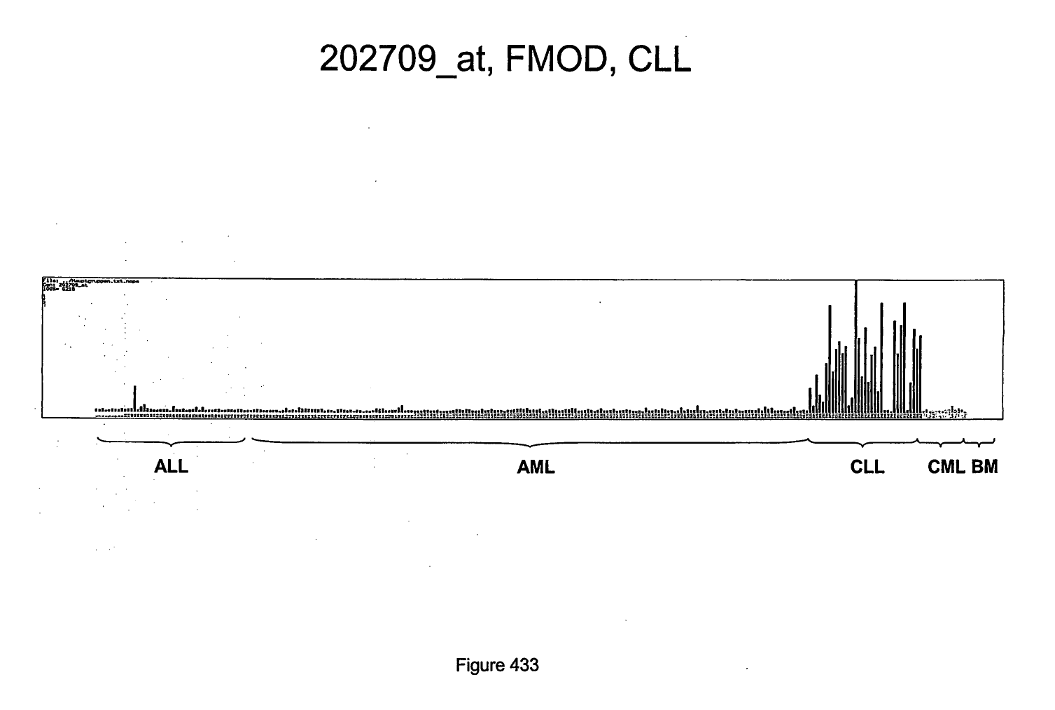

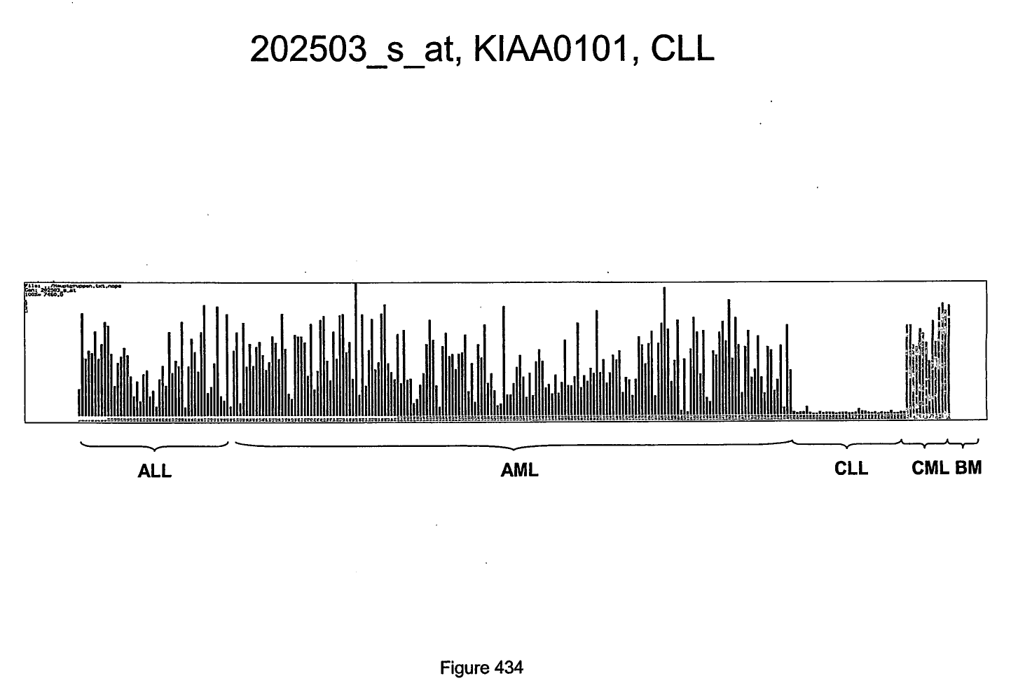

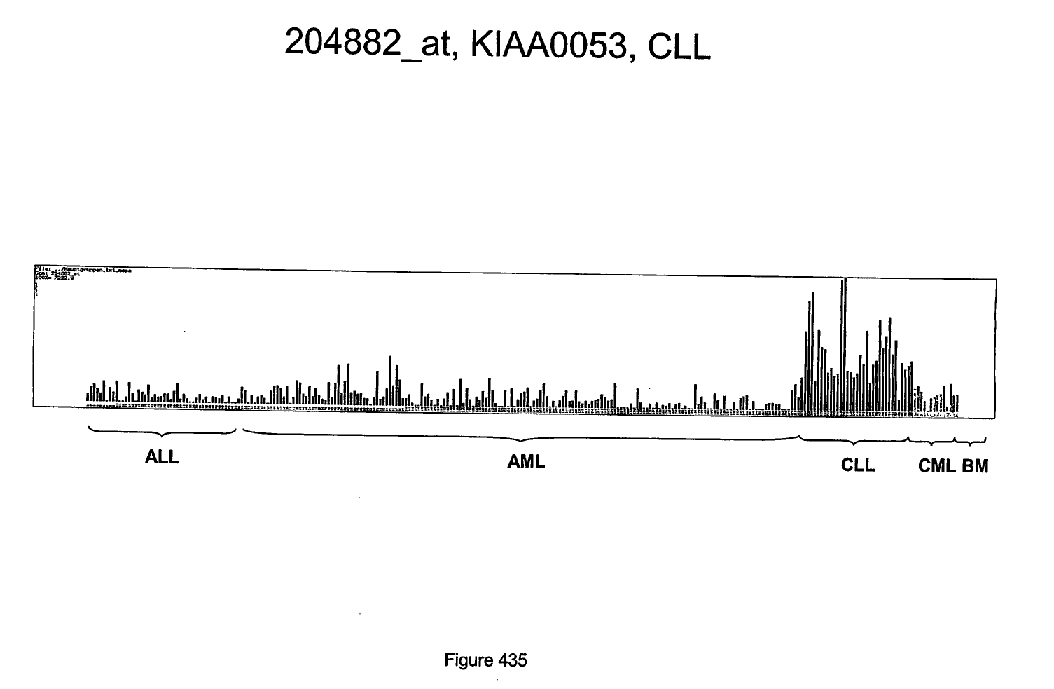

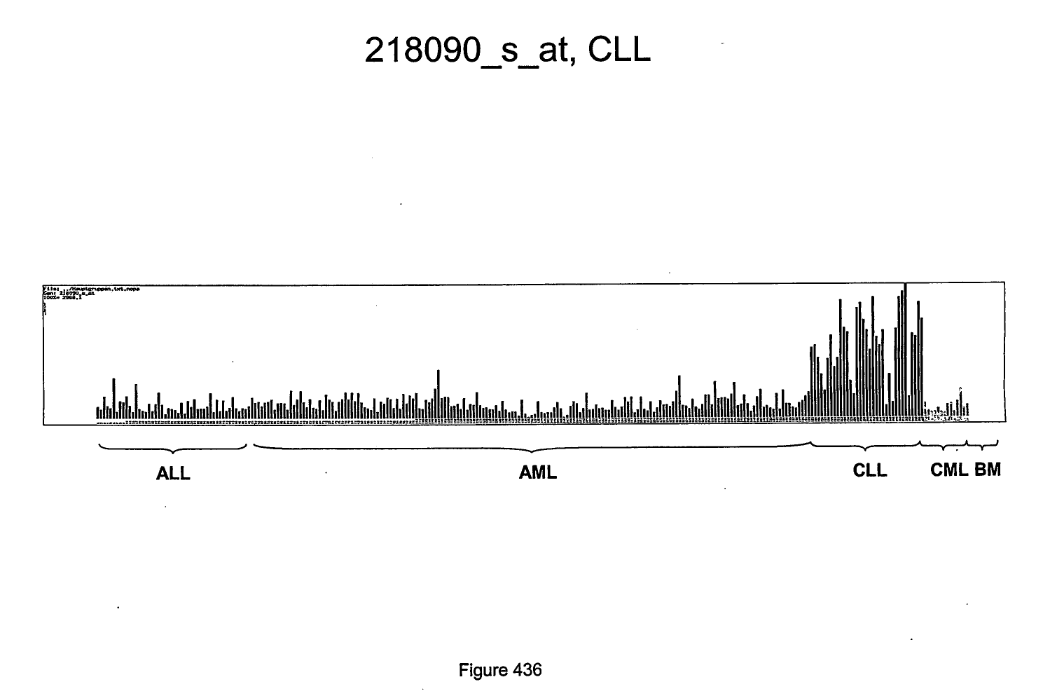

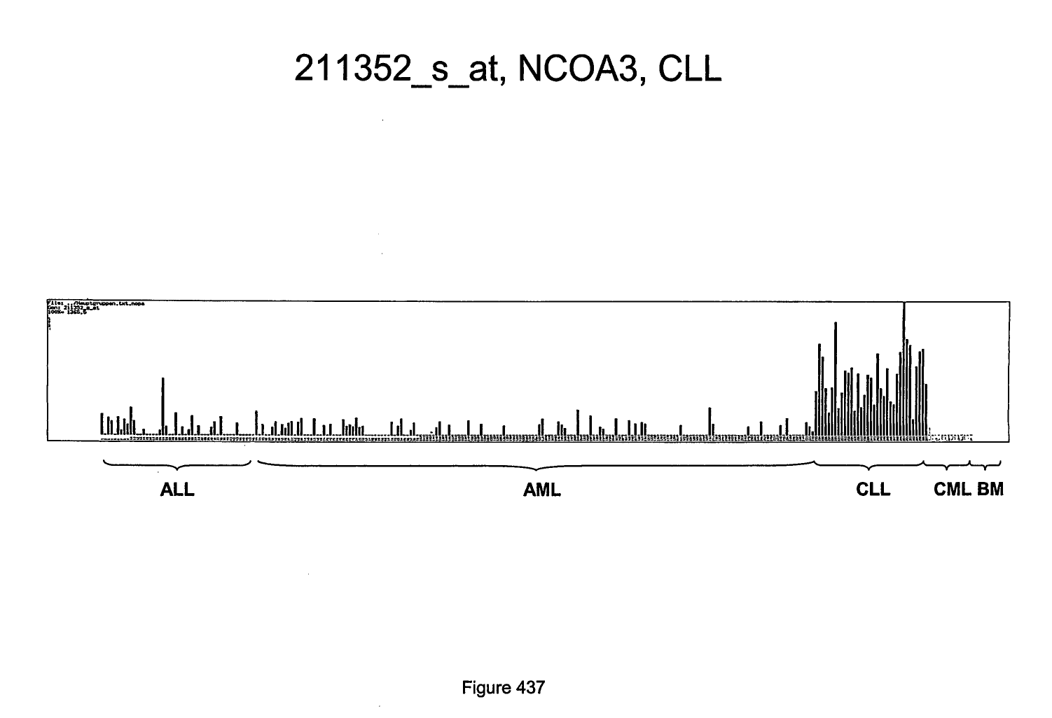

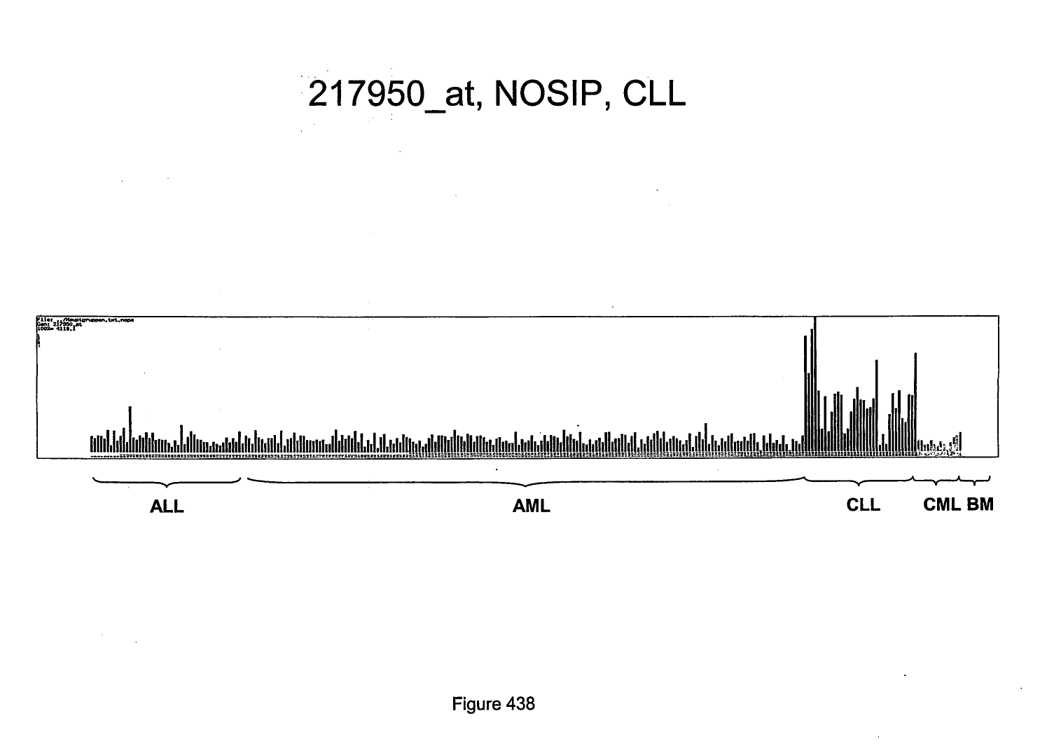

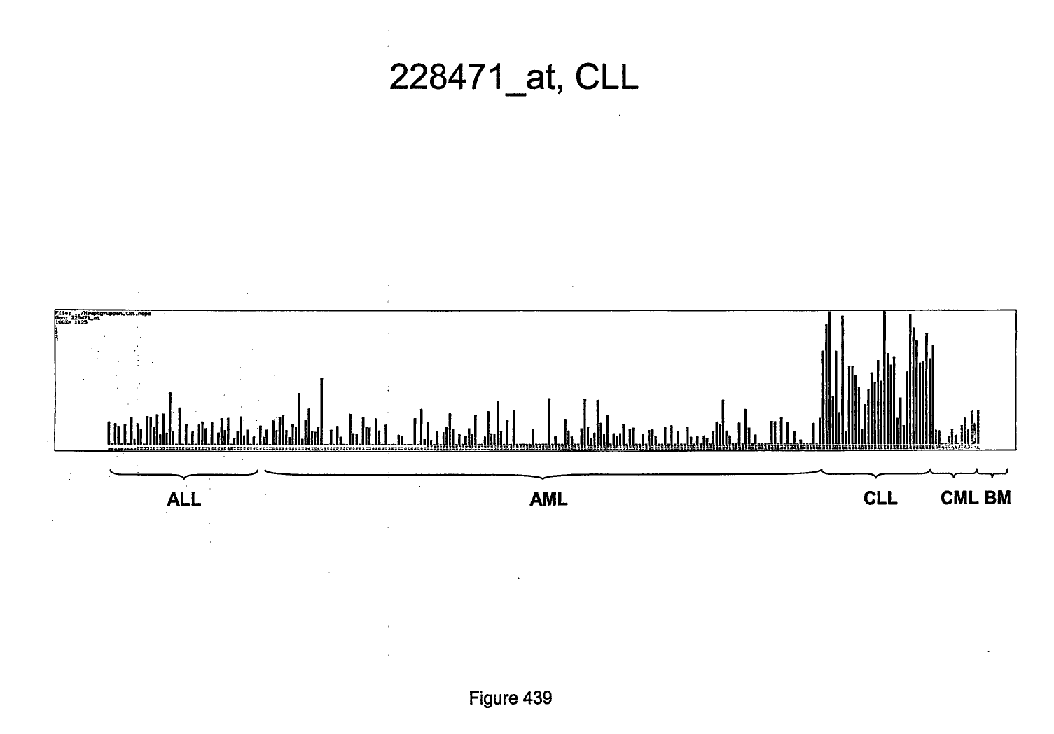

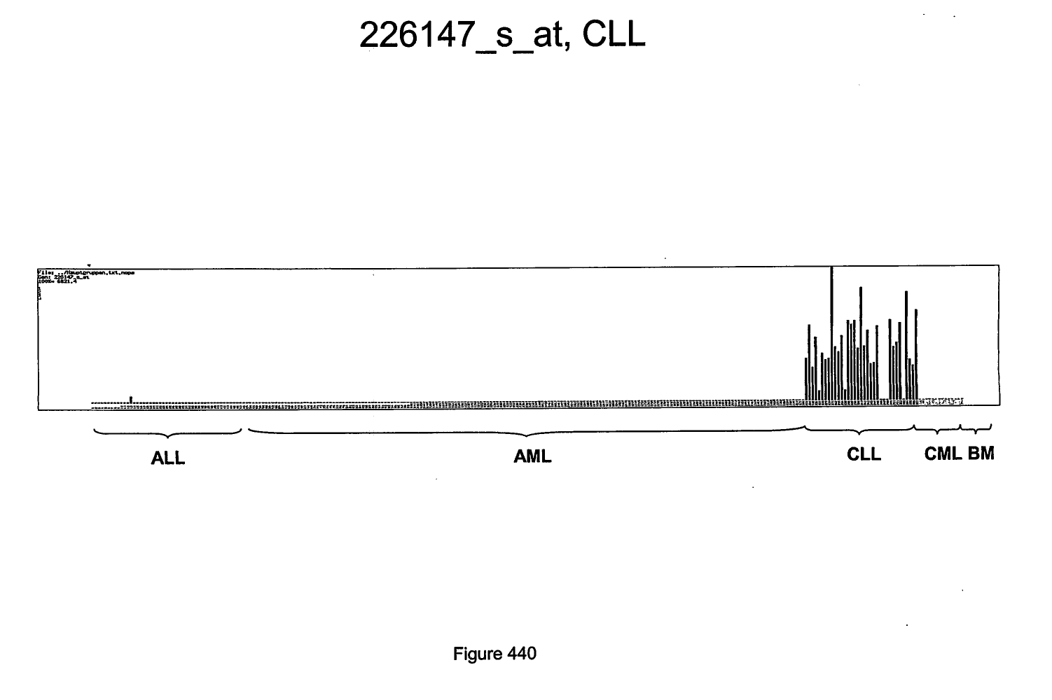

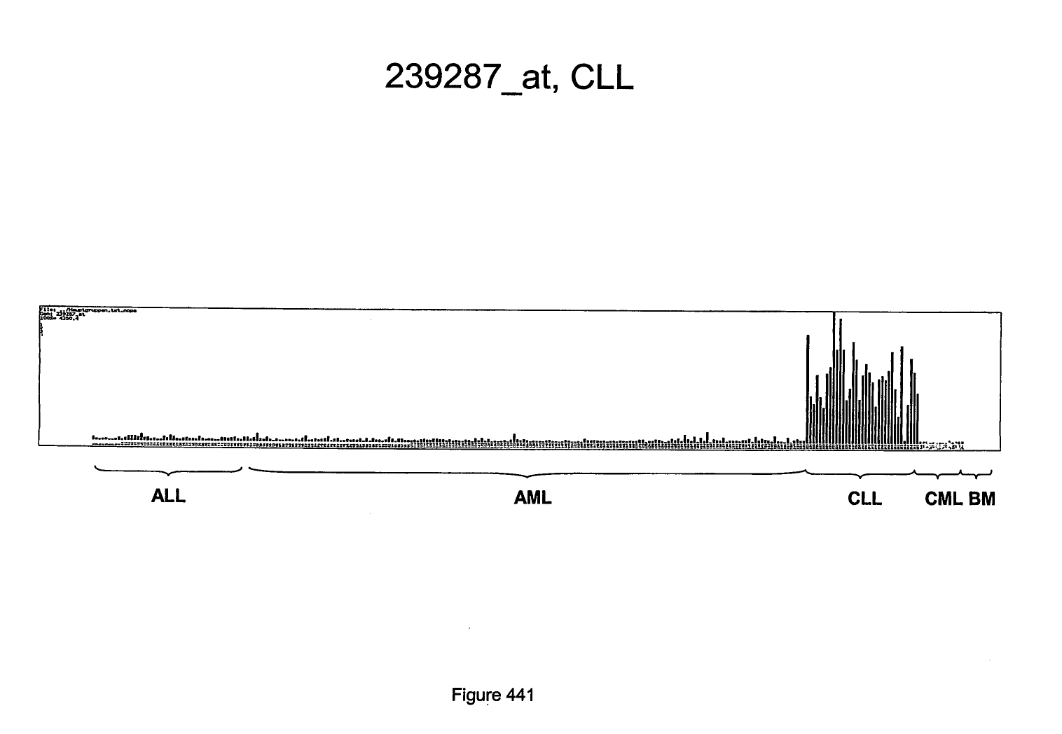

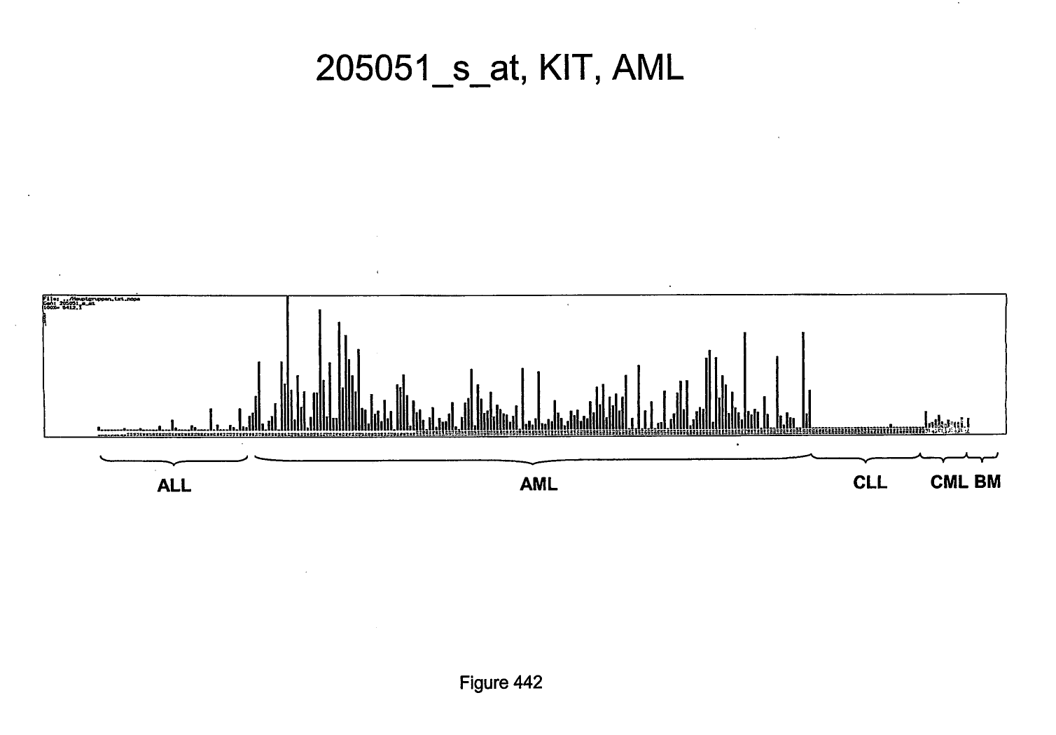

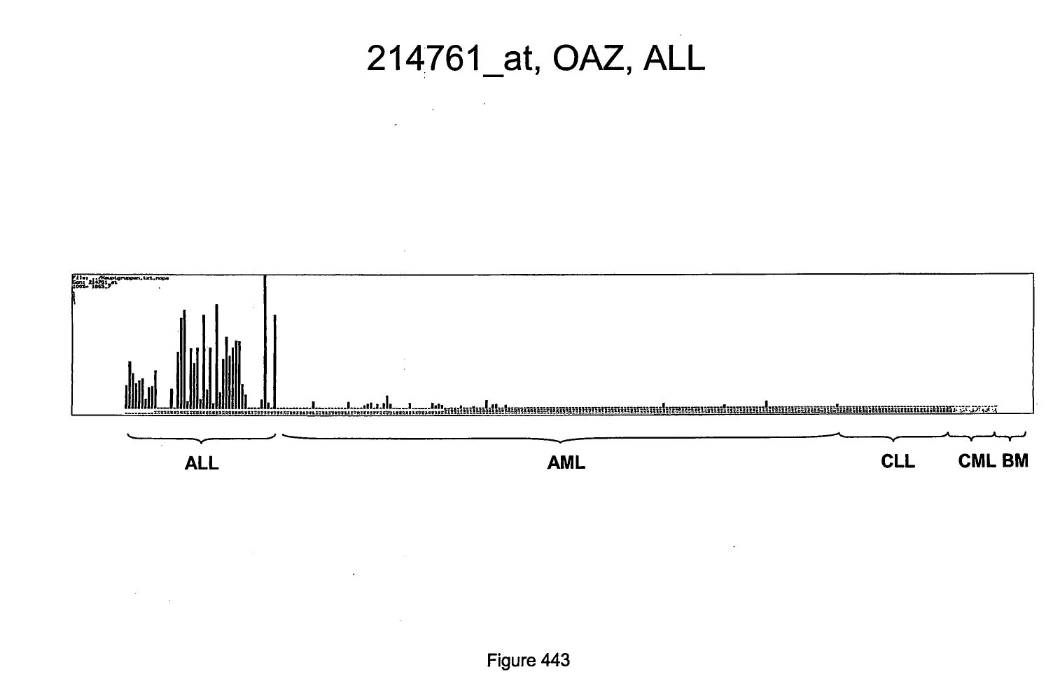

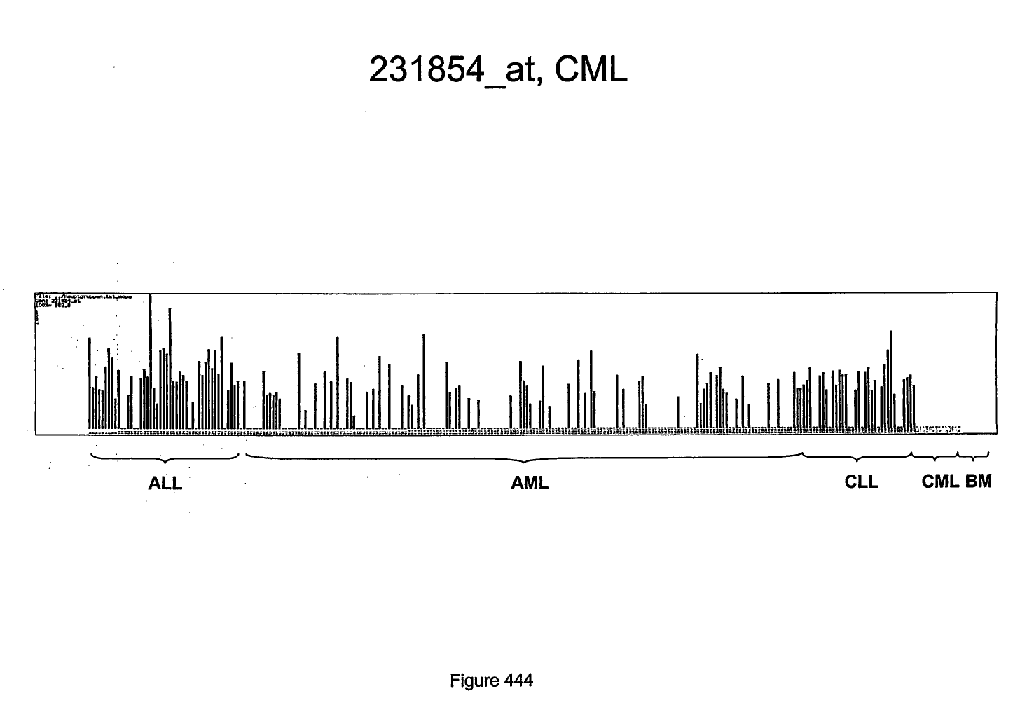

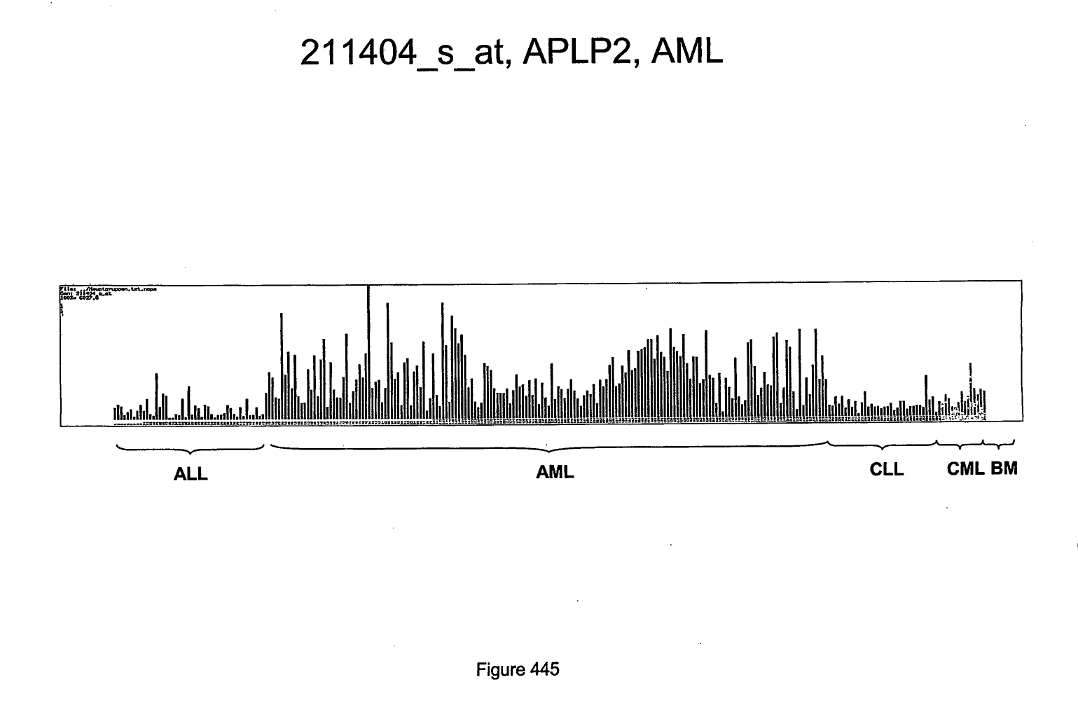

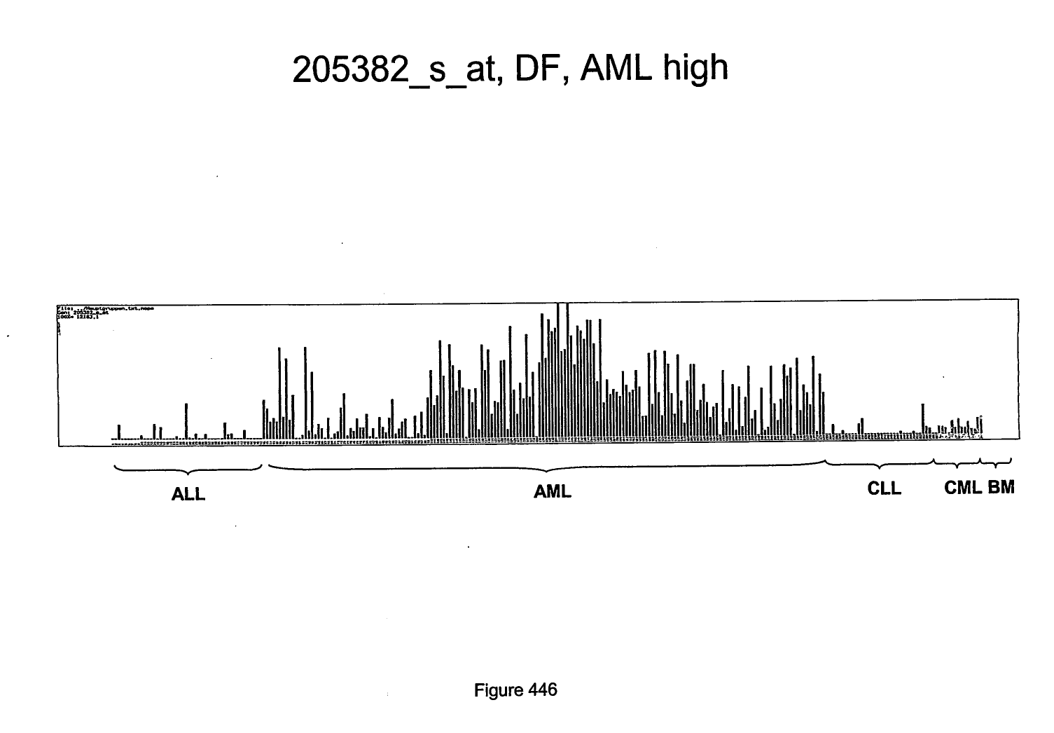

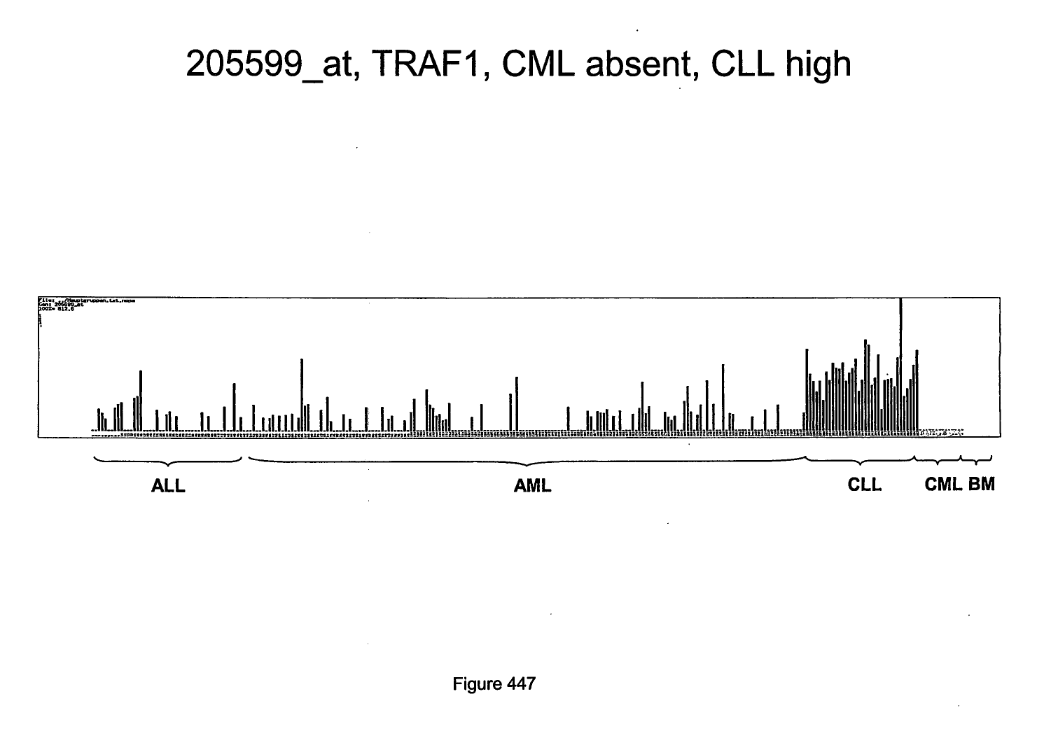

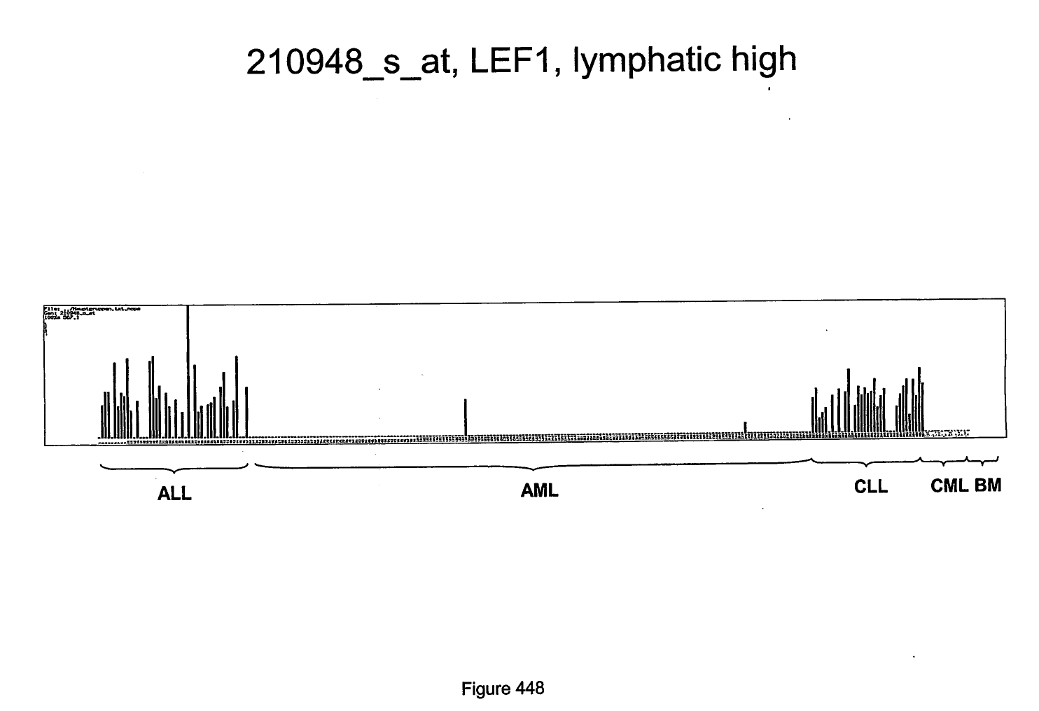

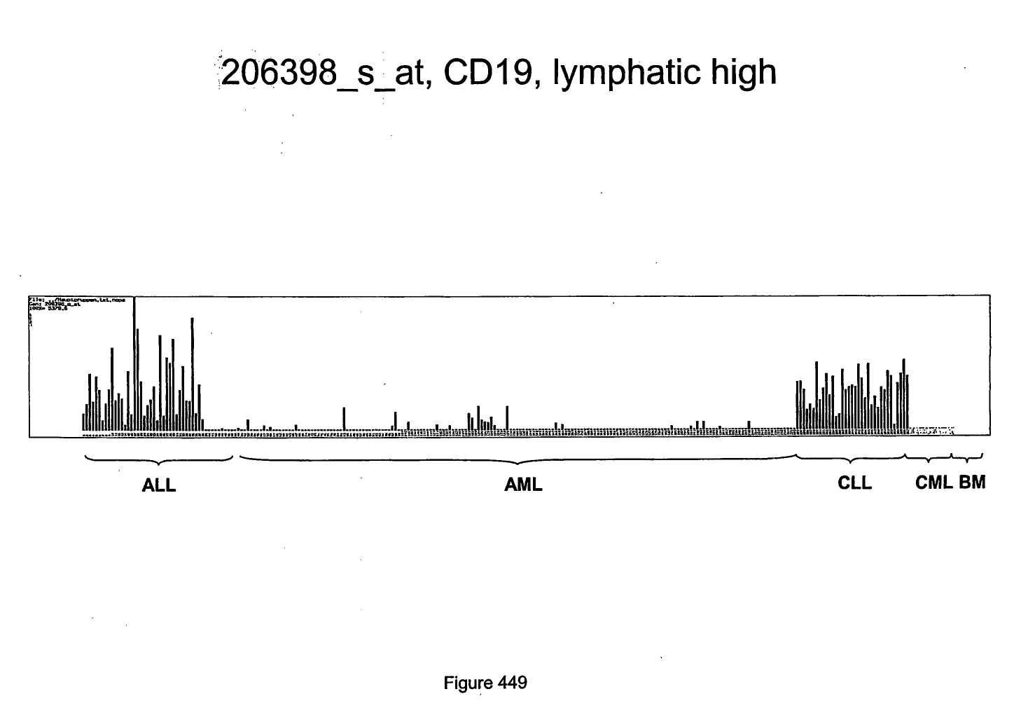

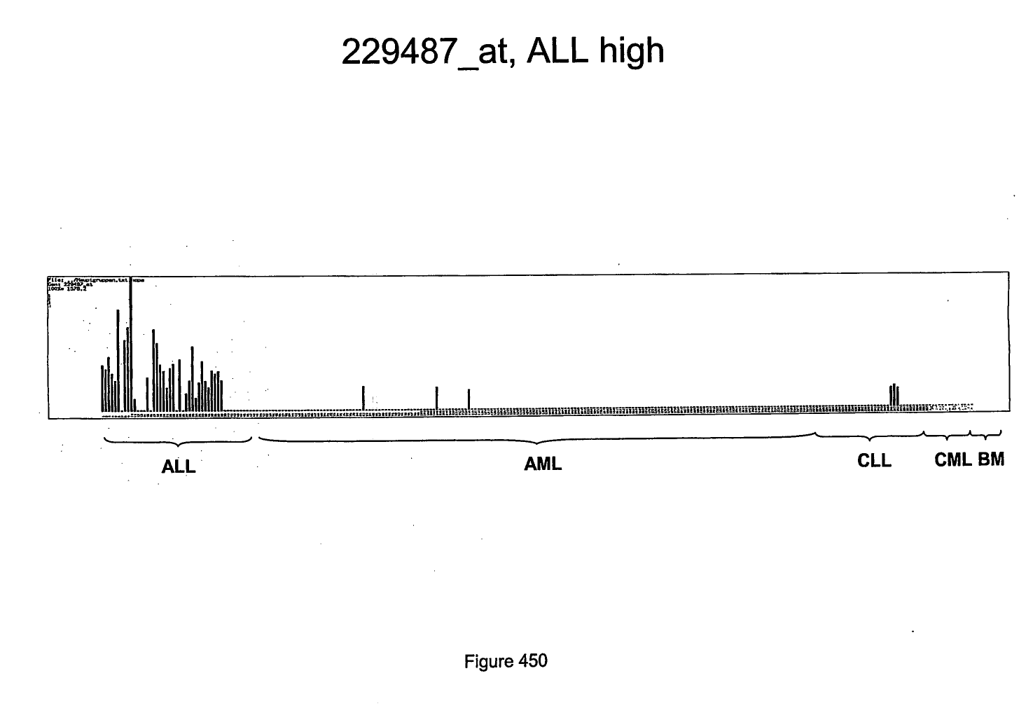

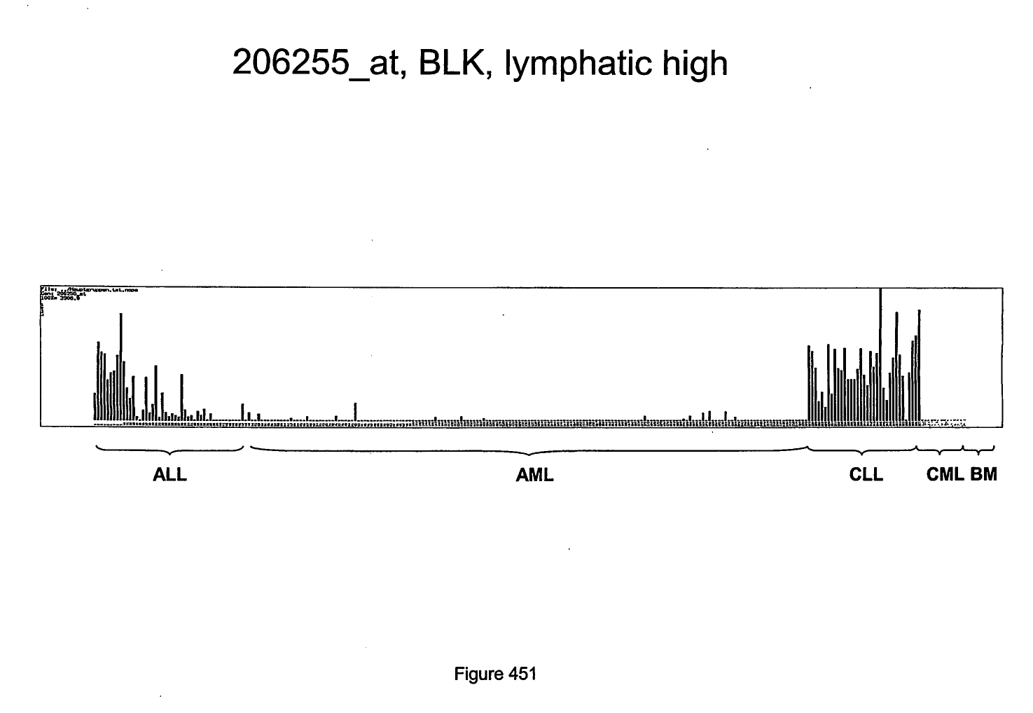

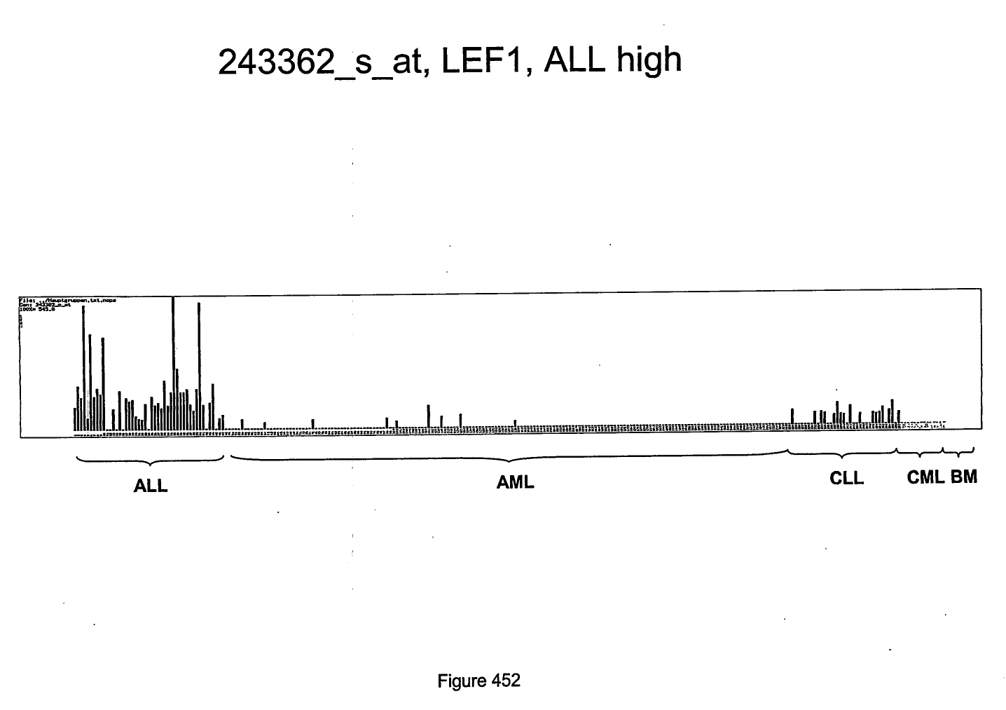

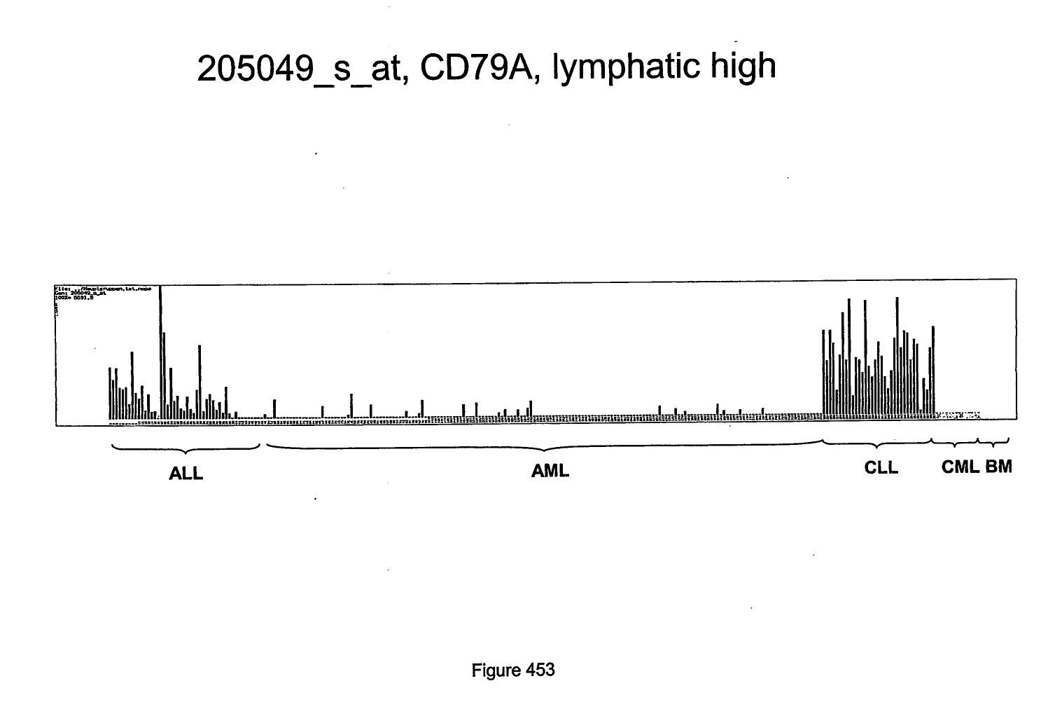

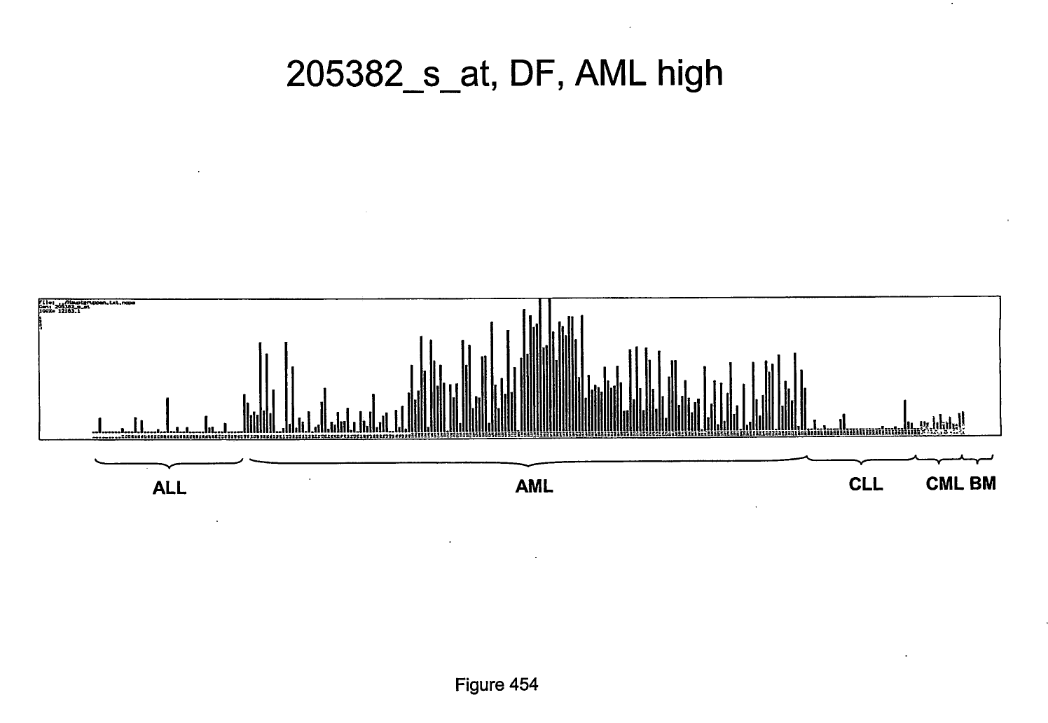

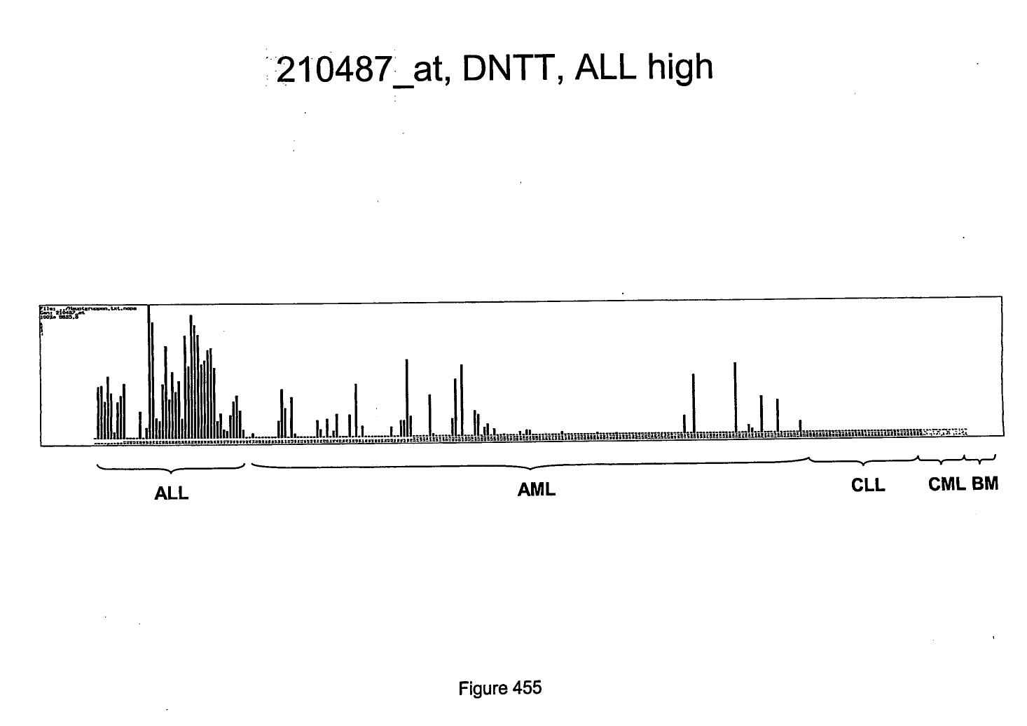

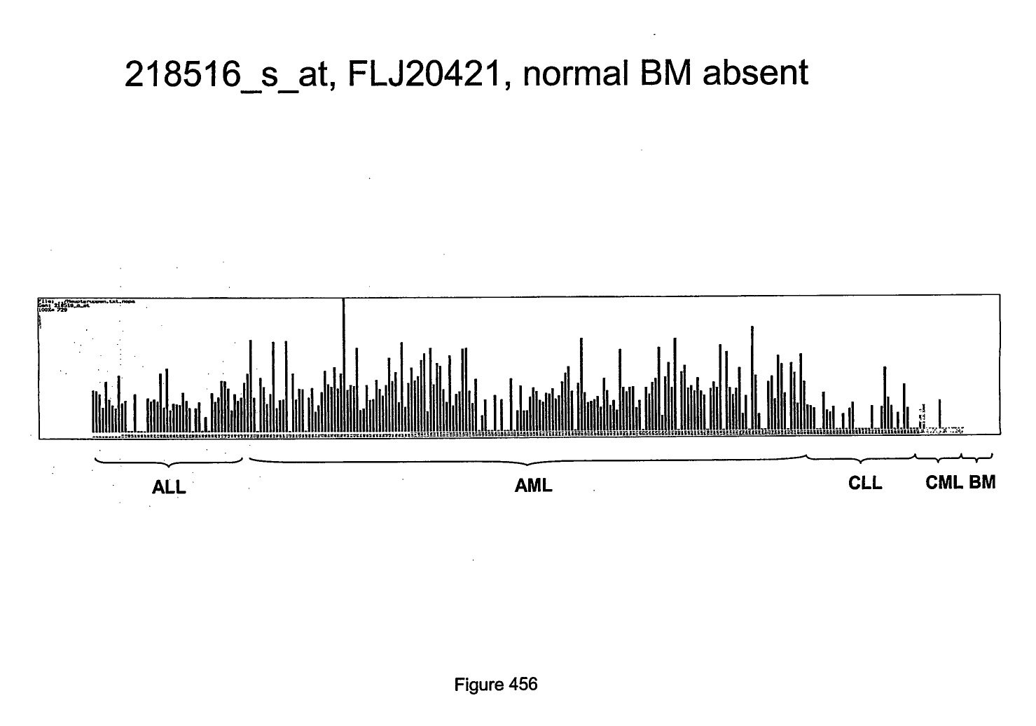

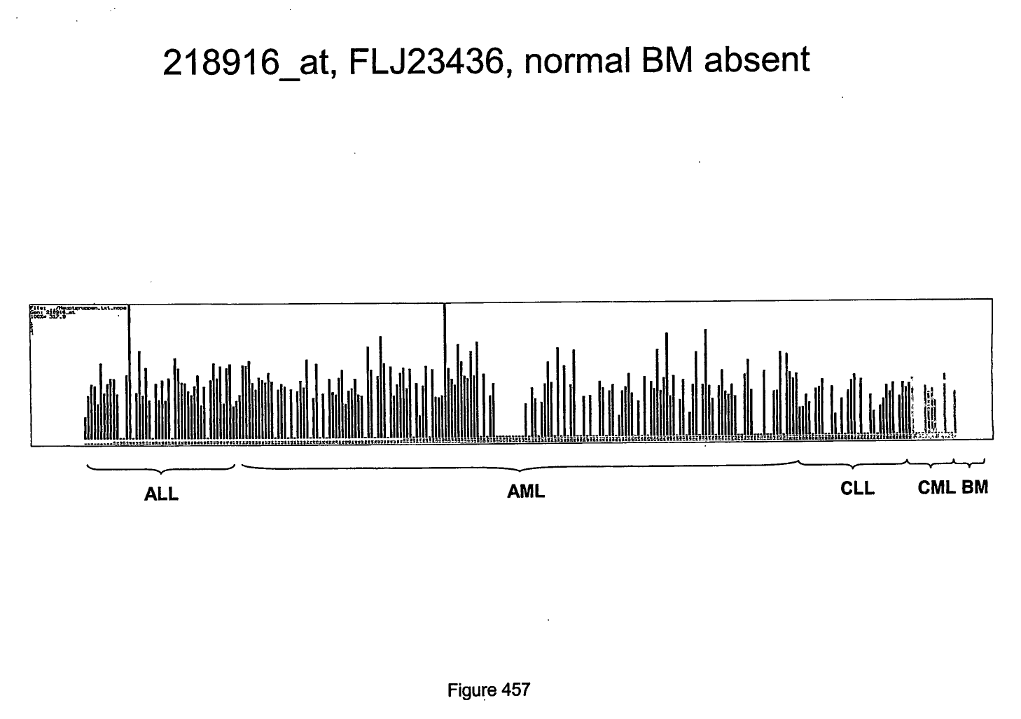

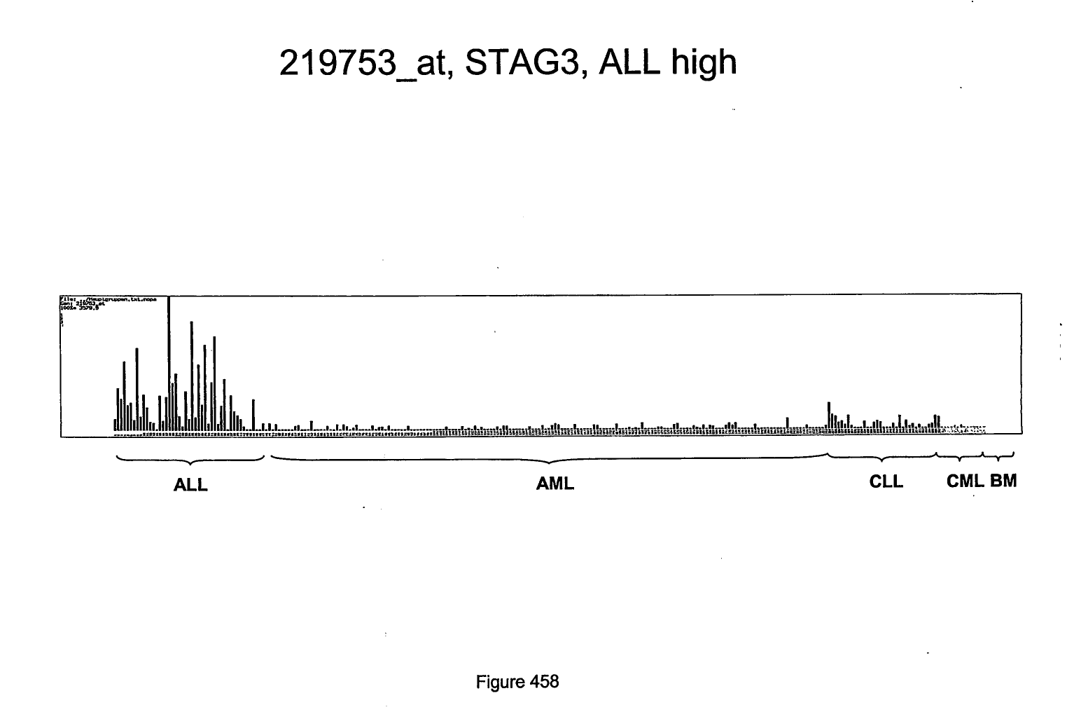

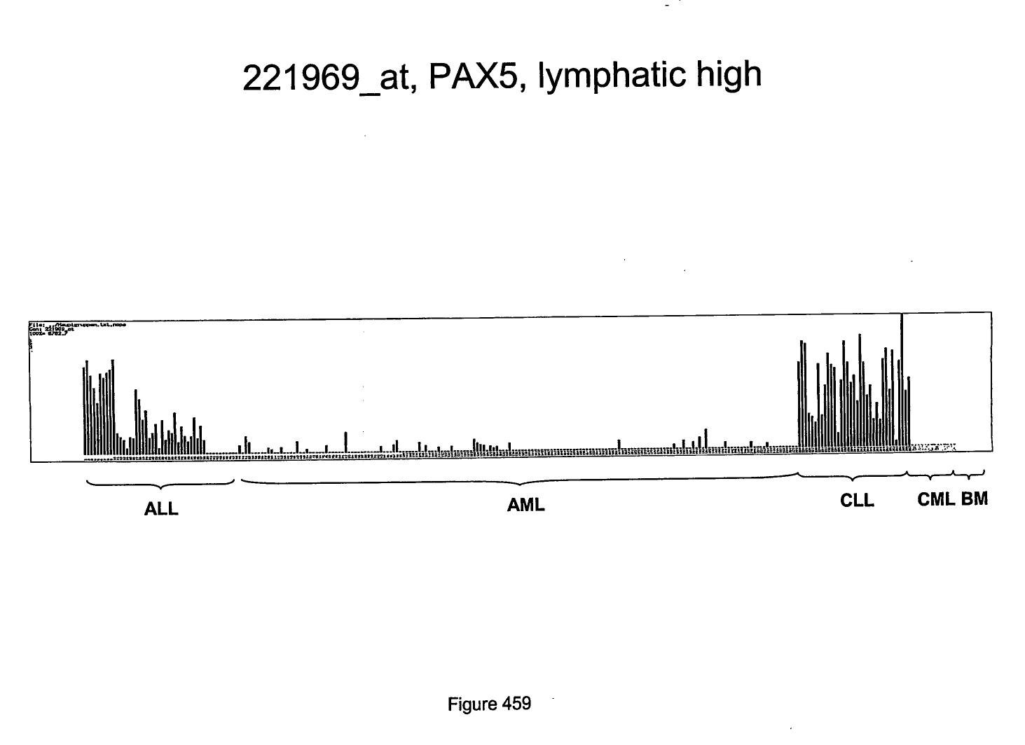

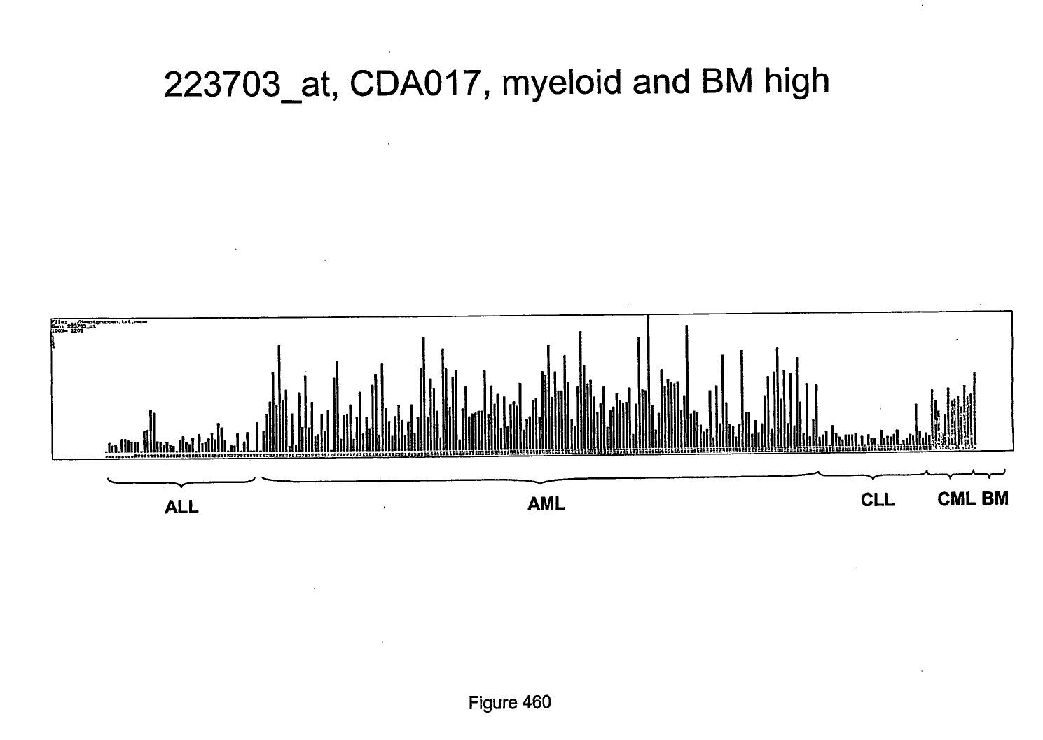

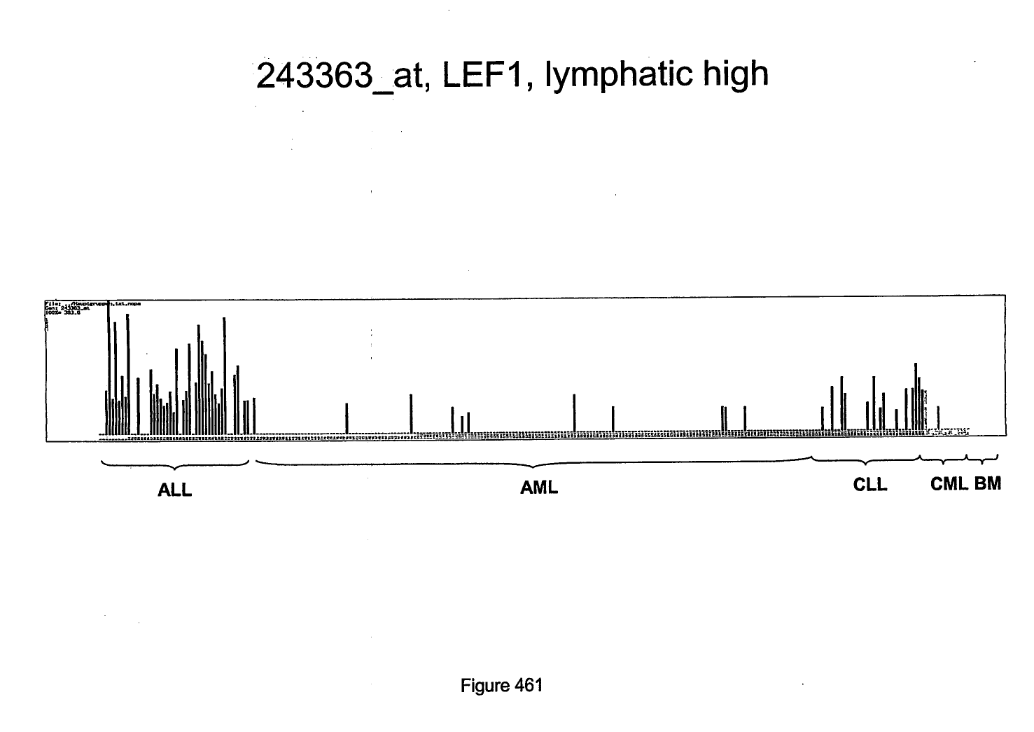

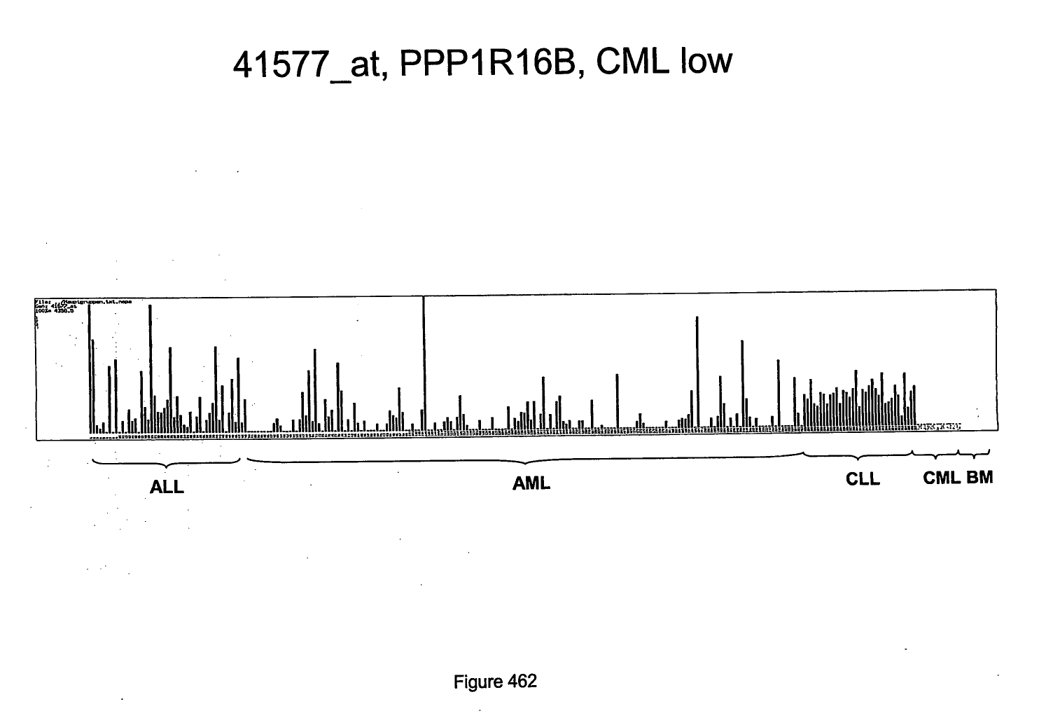

Particularly preferred markers used in a method where only one or a few as e.g. one, preferably two markers are used are described in tables 30, 33, 36 and 42 and Example 7, FIGS. 189 to 234, 254 to 272, 338 to 371, 433 to 465, respectively, or the markers marked with an asterisk in tables 29, 32, 35, 38, and 41 and FIGS. 24 to 188, 235 to 253, 273 to 337, 372 to 405, 406 to 432, respectively as the preferred set of markers. In detail, example 7 mentions (see example 7 for more details) the following markers including their expression level:

| geneID | gene symbol | feature |
|---|---|---|
| 201162_at | IGFBP7 | CLL low |
| 201163_s_at | IGFBP7 | CLL low |
| 201362_at | NS1-BP | CML high |
| 201496_x_at | MYH11 | AML inv(16) high |
| 201497_x_at | MYH11 | AML inv(16) high |
| 201998_at | SIAT1 | CLL high |
| 202095_s_at | BIRC5 | CLL low |
| 203074_at | ANXA8 | AML t(15; 17) high |
| 204150_at | STAB1 | AML t(15; 17) high |
| 204511_at | KIAA0793 | CLL high |
| 205528_s_at | CBFA2T1 | AML t(8; 21) high |
| 205529_s_at | CBFA2T1 | AML t(8; 21) high |
| 205805_s_at | ROR1 | CLL high |
| 206940_s_at | POU4F1 | AML t(8; 21) high |
| 207819_s_at | ABCB4 | CLL high |
| 208091_s_at | DKFZP564K0822 | CLL high |
| 208456_s_at | RRAS2 | CLL high |
| 209061_at | NCOA3 | CLL high |
| 209101_at | CTGF | ALL t(4; 11) high, ALL Ph |
| | | high, T-ALL high |
| 209374_s_at | IGHM | CLL high |
| 209616_s_at | CES1 | AML MLL high |
| 210997_at | HGF | AML t(15; 17) high |
| 212285_s_at | AGRN | AML t(15; 17) high |
| 213539_at | CD3D | T-ALL high |
| 214450_at | CTSW | AML t(15; 17) high |
| 215925_s_at | | ALL t(4; 11) high |
| 218223_s_at | LOC51177 | CML low |
| 222166_at | | AML +8 high |
| 224520_s_at | MGC13168 | ALL t(8; 14) high |
| 224794_s_at | LOC51148 | AML t(15; 17) high |
| 225660_at | SEMA6A | ALL B not Ph high, ALL |
| | | Ph high |
| 226496_at | Homo sapiens, Similar to | ALL high, CLL high |
| | hypothetical protein | |
| | FLJ22611, clone | |
| | MGC: 24716 | |
| | IMAGE: 4277726, | |
| | mRNA, complete cds | |
| 228827_at | Homo sapiens clone | AML t(8; 21) high |
| | 25023 mRNA sequence | |
| 228904_at | ESTs | AML normal high, AML +8 |
| | | high, AML complex high |
| 236301_at | Homo sapiens, clone | CLL high |
| | IMAGE: 3866403, | |
| | mRNA | |
| 236892_s_at | HOXB6 | AML normal high, AML +8 |
| | | high, AML complex high |
| 239214_at | ESTs | ALL t(4; 11) high |
| 239393_at | ESTs | ALL t(4; 11) high |
| 239791_at | HOXB6 | AML normal high, AML +8 |
| | | high |
| 240581_at | ESTs | ALL t(4; 11) high |
| 241464_s_at | ESTs | AML MLL high, AML |
| | | normal high, AML +8 high, |
| | | AML complex high |
| 241525_at | ESTs | AML inv(16) high |
| $$2_s_at | LEF1 | ALL high, CLL high |
| $$_at | CTNS | T-ALL low |
| $$_at | FLJ12442 | AML t(15; 17) high |
| $$05_at | LGALS1 | ALL t(4; 11) high |
| 204044_at | QPRT | ALL t(4; 11) high |
| 205899_at | CCNA1 | ALL t(4; 11) high |
| 209168_at | GPM6B | ALL t(4; 11) high |
| 213539_at | CD3D | T-ALL high |
| 213894_at | KIAA0960 | ALL t(4; 11) high |
| 215925_s_at | | ALL t(4; 11) high |
| 218224_at | PNMA1 | T-ALL high |
| 219463_at | C20orf103 | ALL t(4; 11) high |
| 219631_at | FLJ12929 | T-ALL high |
| 225563_at | ESTs | ALL t(4; 11) high |
| 225592_at | NRM | ALL t(4; 11) high |
| 228083_at | Homo sapiens mRNA; | ALL t(4; 11) high |
| | cDNA DKFZp434I1216 | |
| | (from clone | |
| | DKFZp434I1216) | |
| 228988_at | ZNF6 | T-ALL high |
| 235749_at | | ALL t(8; 14) high |
| 242414_at | ESTs | ALL t(4; 11) high |
| 243756_at | ESTs | ALL t(4; 11) high |
| 201497_x_at | MYH11 | AML inv(16) high |
| 228827_at | Homo sapiens clone | AML t(8; 21) high |
| | 25023 mRNA sequence | |
| 38487_at | FLJ12442 | AML t(15; 17) high |
| 203074_at | ANXA8 | AML t(15; 17) high |
| 205528_s_at | CBFA2T1 | AML t(8; 21) high |
| 205529_s_at | CBFA2T1 | AML t(8; 21) high |
| 206940_s_at | POU4F1 | AML t(8; 21) high |
| 211341_at | POU4F1 | AML t(8; 21) high |
| 201496_x_at | MYH11 | AML inv(16) high |
| 228660_x_at | SEMA4F | other high |
| 202718_at | IGFBP2 | AML t(15; 17) high |
| 205380_at | PDZK1 | other high |
| 202746_at | | AML MLL low |
| 201596_x_at | KRT18 | AML t(8; 21) low |
| 34210_at | CDW52 | AML t(15; 17) low |
| 212850_s_at | LRP4 | AML inv(16) high |
| 228904_at | ESTs | AML t(8; 21) low, AML |
| | | t(15; 17) low, AML inv(16) |
| | | low, AML MLL low |
| 203151_at | MAP1A | AML t(8; 21) low |
| 201137_s_at | HLA-DPB1 | AML t(15; 17) low |
| 200675_at | CD81 | AML inv(16) low |
| 201425_at | ALDH2 | AML t(8; 21) low |
| 202085_at | TJP2 | AML inv(16) low |
| 202619_s_at | PLOD2 | AML MLL low |
| 203092_at | TIMM44 | AML inv(16) low |
| 204425_at | ARHGAP4 | AML t(15; 17) low |
| 205366_s_at | HOXB6 | AML t(8; 21) low, AML |
| | | t(15; 17) low, AML inv(16) |
| | | low, AML MLL low |
| 205472_s_at | DACH | AML MLL high |
| 206761_at | TACTILE | AML MLL low |
| 222166_at | | AML +8 low |
| 222335_at | ESTs | AML MLL low |
| 223318_s_at | MGC10974 | AML complex low |
| 225330_at | Homo sapiens, clone | AML inv(16) low |
| | MGC: 18216 | |
| | IMAGE: 4156235, | |
| | mRNA, complete cds | |
| 231277_x_at | ESTs | AML complex low |
| 635_s_at | PPP2R5B | other low |
| 202503_s_at | KIAA0101 | CLL low |
| 202580_x_at | FOXM1 | CLL low |
| 202709_at | FMOD | CLL high |
| 204882_at | KIAA0053 | CLL high |
| 205049_s_at | CD79A | ALL high, CLL high |
| 205051_s_at | KIT | AML high |
| 205382_s_at | DF | AML high |
| 205599_at | TRAF1 | CML low CLL high |
| 206255_at | BLK | ALL high, CLL high |
| 206398_s_at | CD19 | ALL high, CLL high |
| 210487_at | DNTT | ALL high |
| 210948_s_at | LEF1 | ALL high, CLL high |
| 211352_s_at | NCOA3 | CLL high |
| 211404_s_at | APLP2 | AML high |
| 214761_at | OAZ | ALL high |
| 217950_at | NOSIP | CLL high |
| 218090_s_at | | CLL high |
| 218516_s_at | FLJ20421 | normal BM low |
| 218916_at | FLJ23436 | normal BM low |
| 219753_at | STAG3 | ALL high |
| 221969_at | PAX5 | ALL high, CLL high |
| 223703_at | CDA017 | AML high, CML high, |
| | | normal BM high |
| 226147_s_at | Homo sapiens | CLL high |
| | cDNA: FLJ22667 fis, | |
| | clone HSI08385 | |
| 228471_at | ESTs | CLL high |
| 229487_at | ESTs | ALL high |
| 229790_at | TERF2 | CML low, BM low |
| 231736_x_at | MGST1 | AML high, CML high, |
| | | normal BM high |
| 231854_at | Homo sapiens cDNA | CML low |
| | FLJ11448 fis, | |
| | clone HEMBA1001391 | |
| 239287_at | ESTs | CLL high |
| 243362_s_at | LEF1 | ALL high |
| 243363_at | LEF1 | ALL high, CLL high |
| 41577_at | PPP1R16B | CML low |

Preferred methods for detection and quantification of the amount of nucleic acids, i.e. for the methods according to the invention allowing the determination of the level of expression of a marker or a group of markers, are those described by Sambrook et al. (1989) or real time methods known in the art as the TaqMan® method disclosed in WO92/02638 and the corresponding U.S. Pat. No. 5,210,015, U.S. Pat. No. 5,804,375, U.S. Pat. No. 5,487,972. This method exploits the exonuclease activity of a polymerase, to generate a signal. In detail, the (at least one) target nucleic acid component is detected by a process comprising contacting the sample with an oligonucleotide containing a sequence complementary to a region of the target nucleic acid component and a labeled oligonucleotide containing a sequence complementary to a second region of the same target nucleic acid component sequence strand, but not including the nucleic acid sequence defined by the first oligonucleotide, to create a mixture of duplexes during hybridization conditions, wherein the duplexes comprise the target nucleic acid annealed to the first oligonucleotide and to the labeled oligonucleotide such that the 3′-end of the first oligonucleotide is adjacent to the 5′-end of the labeled oligonucleotide. Then this mixture is treated with a template-dependent nucleic acid polymerase having a 5′ to 3′ nuclease activity under conditions sufficient to permit the 5′ to 3′ nuclease activity of the polymerase to cleave the annealed, labeled oligonucleotide and release labeled fragments. The signal generated by the hydrolysis of the labeled oligonucleotide is detected and/or measured. TaqMan® technology eliminates the need for a solid phase bound reaction complex to be formed and made detectable. Other methods include e.g. fluorescence resonance energy transfer between two adjacently hybridized probes as used in the LightCycler® format described in U.S. Pat. No. 6,174,670.

Protocols for carrying out the methods according to the invention are known to the expert in the field and are described in the examples, preferably in example 1 and 4. A preferred protocol is described in Example 1 (A), where total RNA is isolated, cDNA synthesized and biotin incorporated during the transcription reaction. The purified cDNA was applied to commercially available arrays which can be obtained e.g. from Affymetrix. The hybridized cDNA is detected according to the methods described in Example 1 (A). The arrays are produced by photolithography or other methods known to experts skilled in the art e.g. from U.S. Pat. No. 5,445,934, U.S. Pat. No. 5,744,305, U.S. Pat. No. 5,700,637, U.S. Pat. No. 5,945,334 and EP619 321 or EP 373 203. The latter methods are also suitable for producing the composition according to the inventions in particular the composition wherein polynucleotides or oligonucleotides are bound to a solid phase in particular in the form of arrays. In a further preferred embodiment of the methods according to the invention, a transcribed polynucleotide or portion thereof is the marker or at least one of the markers. A particularly preferred transcribed polynucleotide is an mRNA or a cDNA. In a preferred embodiment of the methods according to the invention, the step of determining the expression profile further comprises amplifying the transcribed polynucleotide. In another preferred embodiment, the level of expression, i.e. the expression profile, of the group of transcribed polynucleotides is determined by annealing the transcribed polynucleotides with a complementary polynucleotide or a portion thereof under stringent hybridization conditions. The term “stringent hyberidisation conditions” is equivalent to the term “highly stringent hyberdisation conditions”. Such highly stringent hybridization conditions may be determined in accordance with the teachings provided in Hames and Higgins (eds) “Nucleic acid hybridization, a practical approach”, IRL Press 1985, Oxford, and include hybridization at 55-65° C. in 0.2-0.5×SSC, 0.1% SDS followed by appropriate washing conditions such as 0.5-1×SSC at 55° C. and 0.1% SDS.

In a most preferred embodiment, the patient sample is blood, i.e. blood mononuclear cells, or bone marrow, i.e. mononuclear cells. The methods according to the invention may be performed on fresh or frozen blood, i.e. blood mononuclear cells or bone marrow, i.e. mononuclear cells.

In a preferred embodiment the marker or at least one of the markers is a protein. In another preferred embodiment the expression profile of the proteins is detected using a reagent which specifically binds to one of the proteins whereby preferably the reagent is selected from the group consisting of an antibody, an antibody derivative, and an antibody fragment.

The term “antibody” comprises monoclonal antibodies as first described by Köhler and Milstein in Nature 278 (1975), 495-497 as well as polyclonal antibodies, i.e. entibodies contained in a polyclonal antiserum. Monoclonal antibodies include those produced by transgenic mice. Fragments of antibodies include F(ab′)₂, Fab and Fv fragments. Derivatives of antibodies include scFvs, chimeric and humanized antibodies. See, for example Harlow and Lane, loc. cit.

Another embodiment of the invention is a kit preferably for assessing the suitability of each of a plurality of compounds for inhibiting leukemia in a patient, the kit optionally comprising the plurality of compounds; and a reagent for assessing the expression profile of a group of markers characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between two and the total number of markers listed in the tables 1 to 20, tables 25 or 27 or tables 29, 30, 32, 33, 35, 36, 38, 39, 41, 42. Another embodiment is a kit preferably for assessing whether a patient is afflicted with leukemia, the kit comprising reagents for assessing the expression profile of a group of markers characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between two and the total number of markers listed in the tables 1 to 20, tables 25 or 27 or tables 29, 30, 32, 33, 35, 36, 38, 39, 41, 42. Another embodiment is a kit preferably for assessing the presence of human leukemia cells, the kit comprising an antibody, wherein the antibody specifically binds with a protein corresponding to a marker characterized in that the marker is selected from the tables 1 to 20, tables 25 or 27 or tables 29, 30, 32, 33, 35, 36, 38, 39, 41, 42. Another embodiment is a kit preferably for assessing the leukemia cell carcinogenic potential of a test compound, the kit comprising leukemia cells and a reagent for assessing expression of a marker, wherein the marker is selected from the tables 1 to 20, tables 25 or 27 or tables 29, 30, 32, 33, 35, 36, 38, 39, 41, 42.

Advantageously, the kit of the present invention further comprises, optionally (a) storage solution(s) and/or remaining reagents or materials required for the conduct of scientific and/or diagnostic and/or therapeutic methods. Furthermore, parts of the kit of the invention can be packaged individually in vials or bottles or in combination in containers or multicontainer units.

Another embodiment of the invention is related to a protein or the RNA, cDNA or cRNA corresponding to a marker selected from the tables 1 to 20, tables 25 or 27 or tables 29, 30, 32, 33, 35, 36, 38, 39, 41, 42 or the use thereof for the treatment of or vaccination against leukemia. Alternatively and depending on the exact purpose, inhibitors of these compounds such as antibodies, fragments or derivatives thereof may be employed for said purpose.

The invention also contemplates a method for the development or preparation of a pharmaceutical composition for the treatment of leukemia characterized in that a protein corresponding to a marker selected from the tables 1 to 20, tables 25 or 27 or tables 29, 30, 32, 33, 35, 36, 38, 39, 41, 42 is admixed with pharmaceutical compounds. Another embodiment of the invention is related to a method for the development or preparation of a pharmaceutical composition for the treatment of leukemia characterized in that a vector comprising a polynucleotide encoding a protein corresponding to a marker selected from the tables 1 to 20, tables 25 or 27 or tables 29, 30, 32, 33, 35, 36, 38, 39, 41, 42 is admixed with pharmaceutical compounds. Another embodiment of the invention is a method for the development or preparation of a pharmaceutical composition for the treatment of leukemia characterized in that an antisense oligonucleotide complementary to a polynucleotide encoding a protein corresponding to a marker selected from the tables 1 to 20, tables 25 or 27 or tables 29, 30, 32, 33, 35, 36, 38, 39, 41, 42 is admixed with pharmaceutical compounds. Alternatively, inhibitors such as antibodies specific for the markers may be used for the preparation or development of a pharmaceutical composition.

The term “pharmaceutical compounds” is preferably to be understood to mean pharmaceutically acceptable carriers, diluents or excipients, only in connection with the embodiments recited in this paragraph. In another embodiment of the invention a marker or a group of markers selected individually from one or more of the tables 1 to 20, tables 25 or 27 or tables 29, 30, 32, 33, 35, 36, 38, 39, 41, 42 is used for the determination of leukemia cells, the type or subtype of leukemia cells.

In another embodiment of the invention a marker or a group of markers selected individually from one or more of the tables 1, 2, 13, 14, 17, 25, 27, 35 or 36 is used for the determination of the subtype of AML cells.

In a preferred embodiment, the invention is related to a composition comprising a group of markers and substances chemically different to the markers characterized in that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one, preferably two and the total number of markers listed in the tables 1 to 20, tables 25 or 27 or tables 29, 30, 32, 33, 35, 36, 38, 39, 41, 42. It is preferred that the composition according to the invention is characterized in that the group of markers consists of all markers listed in one or more of the tables 1 to 20, tables 25 or 27 or tables 29, 30, 32, 33, 35, 36, 38, 39, 41, 42. More preferred the composition according to the invention is characterized in that the group of markers consists of all markers listed in one or more of the tables 14, tables 16 to 20, or table 29 or 30, most preferred the group of markers consists of all markers listed in the tables 16 to 20 or tables 29 or 30. Preferably the markers are polynucletides or oligonucleotides, whereby the polynucleotides are bound to a solid phase in the form of an array.

The present invention also relates to a method of determining the subtypes of ALL cells in a patient sample comprising the steps of a) determining the level of expression of a group of markers in the patient sample and b) concluding from the differences in the level of expression which subtypes of ALL cells the patient sample contains characterized in that the group of markers consists of markers selected independently from the markers listed in one or more of the tables 18, 32 or 33 and whereby the number of markers in the group is between two and the total number of markers listed in the tables 18, 32 or 33.

Preferably the group of markers consists of all markers listed in one or more of the tables 18, 32 or 33.

The present invention further relates to a method of determining the subtypes of CLL cells in a patient sample comprising the steps of a) determining the level of expression of a group of markers in the patient sample and b) concluding from the differences in the level of expression which subtypes of CLL cells the patient sample contains characterized in that the group of markers consists of markers selected independently from the markers listed in one or more of the tables 38 or 39 and whereby the number of markers in the group is between two and the total number of markers listed in the tables 38 or 39.

It is preferred that the group of markers consists of all markers listed in one or more of the tables 38 or 39.

The present invention is also related to a diagnostic composition comprising at least one nucleic acid molecule, preferably (a) single-stranded nucleic acid molecule(s), which is capable of specifically hybridizing to the mRNA of at least one gene listed in Table 1. The use of said nucleic acid molecules for diagnosis of leukemia subtypes, preferably based on microarray technology, offers the following advantages: (1) more rapid and more precise diagnosis, (2) easy to use in laboratories without specialized experience, (3) abolishes the requirement for analyzing viable cells for chromosome analysis (transport problem), (4) very experienced hematologists for cytomorphology and cytochemistry, immunophenotyping as well as cytogeneticists and molecularbiologists are no longer required, and (5) improves the subclassification of leukemia due to the definition of new entities based on gene expression profiles in those subtypes that are not clearly defined with the methods of the prior art (class discovery).

As used herein, the term “capable of specifically hybridizing” has the meaning of hybridization under conventional hybridization conditions, preferably under stringent conditions as described, for example, in Sambrook, J., et al., in “Molecular Cloning: A Laboratory Manual” (1989), Eds. J. Sambrook, E. F. Fritsch and T. Maniatis, Cold Spring Harbour Laboratory Press, Cold Spring Harbour, NY and the further definitions provided above. Also contemplated are nucleic acid molecules that hybridize at lower stringency hybridization conditions. Changes in the stringency of hybridization and signal detection are primarily accomplished through the manipulation, preferably of formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 0.2M NaH2PO4; 0.02M EDTA, pH 7.4), 0.5% SDS, 30% formamide, 100 mg/ml salmon sperm blocking DNA, followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. The inclusion of specific blocking reagents may require modification of the hybridization conditions described above, due to problems with compatibility.

As a hybridization probe (or primer) nucleic acid molecules can be used, for example, that have exactly or basically the nucleotide sequence of at least one of the genes depicted in the appended tables or parts of these sequences. The term nucleic acid molecule as used herein also comprises fragments which are understood to be parts of the nucleic acid molecules that are long enough to specifically hybridize to transcripts of at least one of the genes of the appended tables. These nucleic acid molecules can be used, for example, as probes or primers in a diagnostic assay. Preferably, the nucleic acid molecules of the present invention have a length of at least 8, 10, 12, 13, 15, 18 in particular of at least 20 and particular preferred of at least 25 nucleotides. The nucleic acid molecules of the invention or parts therefrom* can also be used, for example, as primers for a PCR reaction. The fragments used as hybridization probe can be synthetic fragments that were produced by means of conventional synthesis methods.

In a preferred embodiment, the diagnostic composition of the present invention comprises at least nucleic acid molecules which are capable of specifically hybridizing to the mRNAs of at least one of the genes listed in the appended tables, preferably 2-5, more preferably 8-12 genes.

In a more preferred embodiment, the diagnostic composition of the present invention comprises at least nucleic acid molecules which are capable of specifically hybridizing to the mRNAs of at least one of the genes listed in the appended tables. In a further preferred embodiment, the diagnostic composition of the present invention comprises at least nucleic acid molecules which are capable of specifically hybridizing to the mRNAs of all genes listed in the appended tables.

In a further preferred embodiment, the nucleic acid molecules of the diagnostic composition of the present invention are bound to (a) a solid support, for example, a polystyrene microtiter dish or nitrocellulose membrane or glass surface or (b) to non-immobilized particles in solution.

In an even more preferred embodiment, the nucleic acid molecules of the diagnostic composition are present in a microarray format which can be established according to well known methods; for details see, e.g., www.affymetrix.com/technology/tech_spotted.html; www.affymetrix.com/technology/tech_probe.html.

The present invention also provides the use of (a) nucleic acid molecule(s) of the present invention for the preparation of a diagnostic composition for the diagnosis of a leukemia or for the diagnosis of several subtypes or a disposition to a leukemia. For the diagnosis of a particular leukemia subtype, preferably, at least 5 different nucleic acid molecules are used as probes. For diagnosis, preferably, bone marrow or peripheral blood can be used. For diagnosis, the target sample is contacted with a (a) nucleic acid molecule(s) of the present invention and the concentration of individual mRNAs is compared with the mRNA expression profile levels of a test sample obtained from healthy donors.

It is a further embodiment of the invention to provide a method of determining whether a patient sample contains leukemia cells or other cells and at the same time determining the type and subtype of leukemia cells, comprising the steps of providing a patient sample, isolating RNA from the patient sample, transcribing the RNA into cDNA and transcribing the cDNA into cRNA while simultaneously labelling the cRNA, hybridising the cRNA to a microarray, and determining the level of expression of a marker or a group of markers.

Further, the invention contemplates the use of a marker or a group of markers for determining whether a patient sample contains leukemia cells or other cells and whereby preferably the type and subtype of leukemia cells is simultaneously or subsequently is determined. The markers specified in the appended examples and tables may, in accordance with the invention, be used to differentiate, for example, between ALL, CLL, CML and AML.

The nucleic acid molecule is typically a nucleic acid probe for hybridization or a primer for PCR. The person skilled in the art is in a position to design suitable nucleic acids probes based on the information provided in the appended tables.

The target cellular component, i.e. mRNA e.g., in bone marrow or blood (BM) may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP 0 237 362)) and other detection assays that are known to those skilled in the art. Preferably, detection is based on a microarray.

Amplification methods include the polymerase chain reaction (PCR) which specifically amplifies target sequences to detectable amounts. Other possible amplification reactions are the ligase Chain Reaction (LCR, Wu and Wallace, 1989, Genomics 4:560-569 and Barany, 1991, Proc. Natl. Acad. Sci. USA 88:189-193); Polymerase Ligase Chain Reaction (Barany, 1991, PCR Methods and Applic. 1:5-16); Gap-LCR(PCT Patent Publication No. WO 90/01069); Repair Chain Reaction (European Patent Publication No. 439, 182 A2), 3SR (Kwoh et al., 1989, Proc. Natl. Acad. Sci. USA 86:1173-1177; Guatelli et al., 1990, Proc. Natl. Acad. Sci. USA 87:1874-1878; PCT Patent Publication No. WO 92/0880A), and NASBA (U.S. Pat. No. 5,130,238). Further, there are strand displacement amplification (SDA), transcription mediated amplification (TMA), and Q□-amplification (for a review see e.g. Whelen and Persing (1996). Annu. Rev. Microbiol. 50, 349-373; Abramson and Myers, 1993, Current Opinion in Biotechnology 4:41-47).

Products obtained by in vitro amplification can be detected according to established methods, e.g. by separating the products on agarose gels and by subsequent staining with ethidium bromide. Alternatively, the amplified products can be detected by using labeled primers for amplification or labeled dNTPs.

The probes can be delectably labeled, for example, with a radioisotope, a bioluminescent compound, a chemiluminescent compound, a fluorescent compound, a metal chelate, biotin or an enzyme.

The invention further contemplates a method of making an isolated hybridoma which produces an antibody useful for assessing whether a patient is afflicted with leukemia, the method comprising isolating a protein corresponding to a marker selected from the group consisting of the markers listed in Tables 1 to 20, tables 25 or 27 or tables 29, 30, 32, 33, 35, 36, 38, 39, 41, 42 immunizing a mammal using the isolated protein, or a peptide corresponding to its sequence or a part thereof; isolating splenocytes from the immunized mammal-, fusing the isolated splenocytes with an immortalized cell line to form hybridomas; and screening individual hybridomas for production of an antibody which specifically binds with the protein to isolate the hybridoma. Further, an antibody produced by this method is contemplated by the invention. The antibody may be fragmented or derivated to obtained fragment or derivatives retaining the antibody specificity as has been described herein above.

The invention further contemplates a method of assessing the leukemia cell carcinogenic potential of a test compound, the method comprising maintaining separate aliquots of leukemia cells in the presence and absence of the test compound; and comparing expression of a marker in each of the aliquots, wherein a significantly altered level of expression of the marker in the aliquot maintained in the presence of the test compound, relative to the aliquot maintained in the absence of the test compound, is an indication that the test compound possesses human breast cell carcinogenic potential wherein a marker according to the invention is used.

The invention further contemplates a system for identifying selected polynucleotide records that identify a leukemia cell, the system comprising: a digital computer-, a database coupled to the computer; a database coupled to the database server having data stored in, the data comprising records of data comprising a polynucleotide, corresponding to a marker according to the invention and a code mechanism for applying queries based upon a desired selection criteria to the data file in the database to produce reports of polynucleotide records which match the desired selection criteria.

The invention also relates to a method for detecting a leukemia cell, using a computer having a processor, memory, display, and input/output devices, the method comprising the steps of

a) providing a sequence of a polynucleotide isolated from a sample suspected of containing a leukemia cell,

b) providing a database comprising records of data comprising a polynucleotide corresponding to a group of markers according to the invention;

c) using a code mechanism for applying queries based upon a desired selection criteria to the data file in the database to produce reports of polynucleotide records of step a) which provide a match of the desired selection criteria of the sequences in the database of step b), the presence of a match being a positive indication that the polynucleotide of step 1) has been isolated from a cell that is a-leukemia cell.

Also, the present invention relates to a method for assessing the leukemia cell carcinogenic potential of a test compound, comprising (a) contacting a non-leukemia cell with a test compound, and (b) assessing an increase or decrease of marker expression in said non-leukemia cell wherein the marker is selected from the tables 1 to 20, 25 or 27, 29, 30, 32, 33, 35, 36, 38, 39, 41 or 42.

The assessment may be effected on the nucleic acid level such as by hybridization techniques or PCR or on the protein level such as by using antibody or aptamers based technologies.

Finally, the invention relates to a diagnostic composition comprising at least one nucleic acid molecule which is capable of specifically hybridizing to the mRNA corresponding to the marker gene of any of the appended tables. The nucleic acid molecule may be an antisense DNA or RNA an RNAi molecule a siRNA molecule or the like inhibitory molecule capable of specifically blocking transcription and/or translation and/or modification and/or localization of the RNA and/or protein corresponding to the marker gene.

The nucleic acid may also be a sense-strand nucleic acid e.g. RNA or preferably DNA which is capable of expressing the protein product of the marker gene, or a protein product of substantially similar activity, in a target cell into which it is introduced.

The invention further comprises pharmaceutical compositions comprising a compound capable of specifically binding to a protein or RNA corresponding to a marker of the invention as listed in any of the appended tables. The marker is preferably selected from the markers designated as particular preferred markers as described herein above. The compound is preferably a compound capable of inhibiting or increasing the function of the protein or of enhancing or decreasing translation of the RNA. The compound is preferably selected from aptamers, aptazynes, RNAzynes, antibodies, affybodies, trinextins, anticalins, or the like compounds. The effect of the compounds on the RNA may be tested by assaying for increased/decreased synthesis of the corresponding protein. The effect of the compounds on the protein may be assayed the testing the effect of the compound in an assay of the proteins function, which e.g. may be an anzymathic function. Alternatively, the effect may be tested by contacting a leukemic cell that expresses large amounts of such protein with the compound and assay cellular parameters associated with the leukemic state of the cell, such as cell growth, growth factor dependency and/or differentiation state of the cell.

In a further embodiment, the invention provides a method of determining whether a patient sample contains leukemia cells or other cells comprising the steps of
a) determining the expression profile of a group of markers in a patient sample and
b) concluding from the expression profile whether the patient sample contains leukemia cells or other cells, and optionally, to which subtype said leukemia cells belong, wherein
   a subtype or a type of leukemia listed in table 28 b or c is identified, and a sensitivity and/or specificity of at least 80, 85, 88, 90, 92, 95, 97, 98, 99, 99.1, 99.2, 99.3, 99.4 or 99.5% is achieved, preferably using at least one marker of the group of markers listed in table 29 and/or 30.

In a further embodiment, the invention provides a method of determining whether a patient sample contains leukemia cells or other cells comprising the steps of
(a) determining the expression profile of a group of markers in a patient sample and
(b) concluding from the expression profile whether the patient sample contains leukemia cells or other cells, and optionally, to which subtype said leukemia cells belong, wherein
a subtype or a type of leukemia listed in table 31 b or c is identified, and a sensitivity and/or specificity of at least 80, 85, 88, 90, 92, 95, 97, 98, 99, 99.1, 99.2, 99.3, 99.4 or 99.5% is achieved, preferably using at least one marker of the group of markers listed in table 32 and/or 33.

In a further embodiment, the invention provides a method of determining whether a patient sample contains leukemia cells or other cells comprising the steps of
(a) determining the expression profile of a group of markers in a patient sample and
(b) concluding from the expression profile whether the patient sample contains leukemia cells or other cells, and optionally, to which subtype said leukemia cells belong, wherein
a subtype or a type of leukemia listed in table 34 b or c is identified, and a sensitivity and/or specificity of at least 80, 85, 88, 90, 92, 95, 97, 98, 99, 99.1, 99.2, 99.3, 99.4 or 99.5% is achieved, preferably using at least one marker of the group of markers listed in table 35 and/or 36.

In a further embodiment, the invention provides a method of determining whether a patient sample contains leukemia cells or other cells comprising the steps of
(a) -determining the expression profile of a group of markers in a patient sample and
(b) concluding from the expression profile whether the patient sample contains leukemia cells or other cells, and optionally, to which subtype said leukemia cells belong, wherein
a subtype or a type of leukemia listed in table 37 b or c is identified, and a sensitivity and/or specificity of at least 80, 85, 88, 90, 92, 95, 97, 98, 99, 99.1, 99.2, 99.3, 99.4 or 99.5% is achieved, preferably using at least one marker of the group of markers listed in table 38 and/or 39.

In a further embodiment, the invention provides a method of determining whether a patient sample contains leukemia cells or other cells comprising the steps of
(a) determining the expression profile of a group of markers in a patient sample and
(b) concluding from the expression profile whether the patient sample contains leukemia cells or other cells, and optionally, to which subtype said leukemia cells belong, wherein
a subtype or a type of leukemia listed in table 40 b or c is identified, and a sensitivity and/or specificity of at least 80, 85, 88, 90, 92, 95, 97, 98, 99, 99.1, 99.2, 99.3, 99.4 or 99.5% is achieved, preferably using at least one marker of the group of markers listed in table 41 and/or 42.

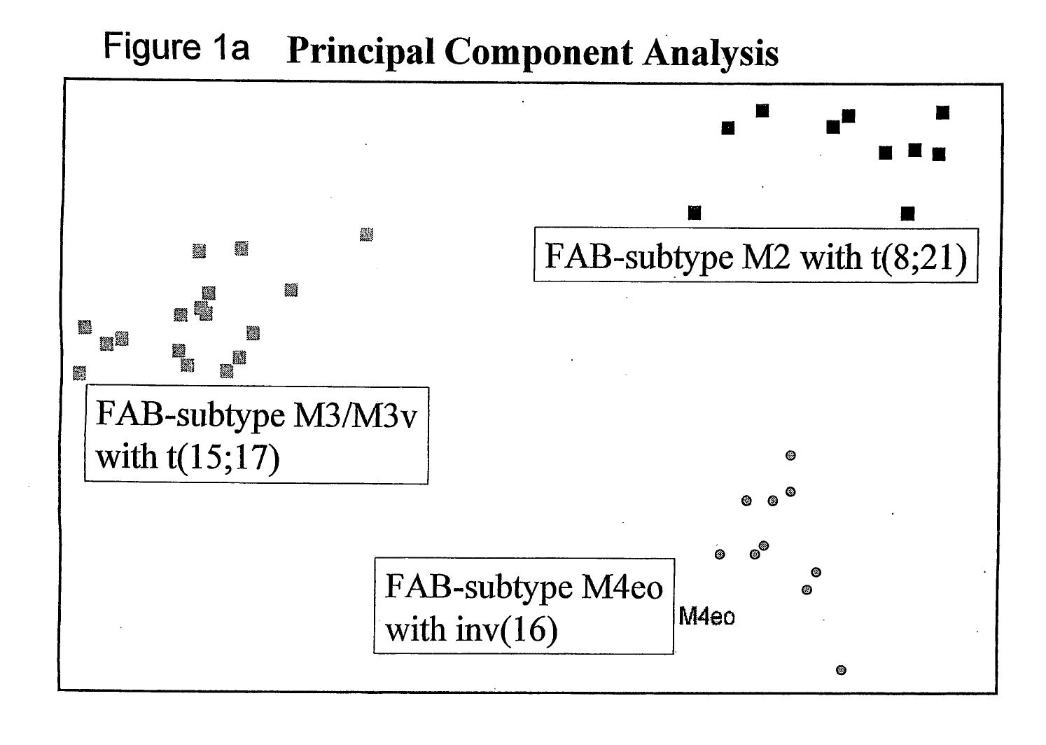

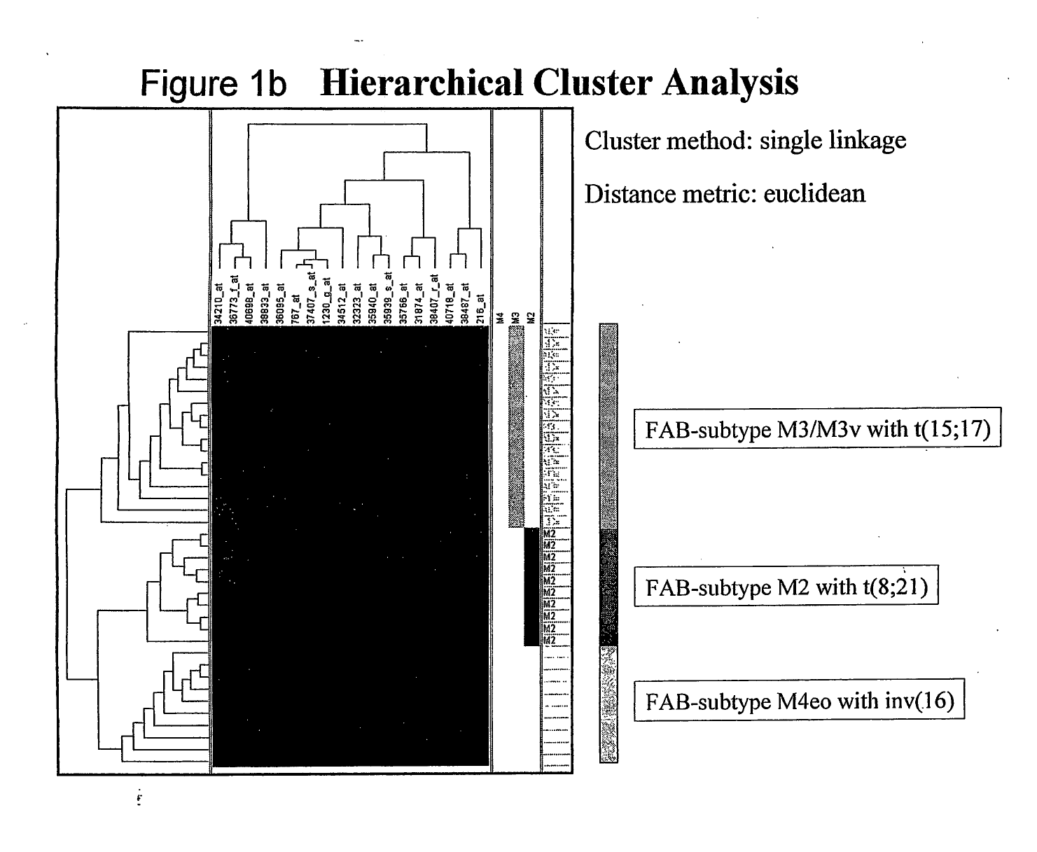

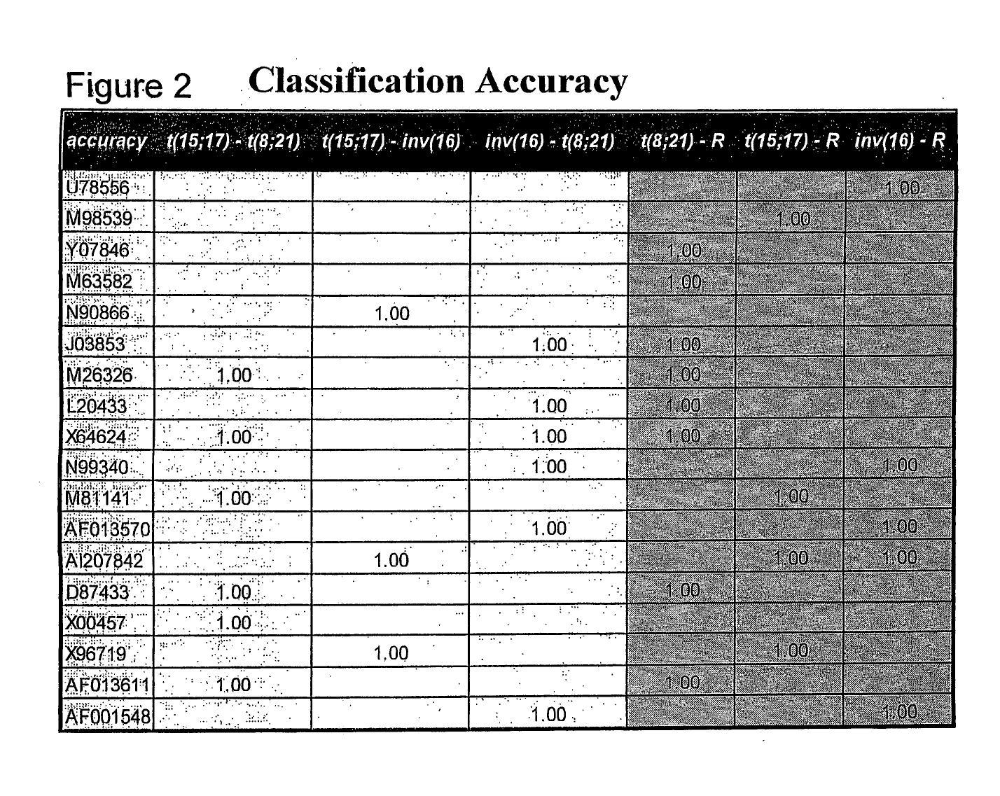

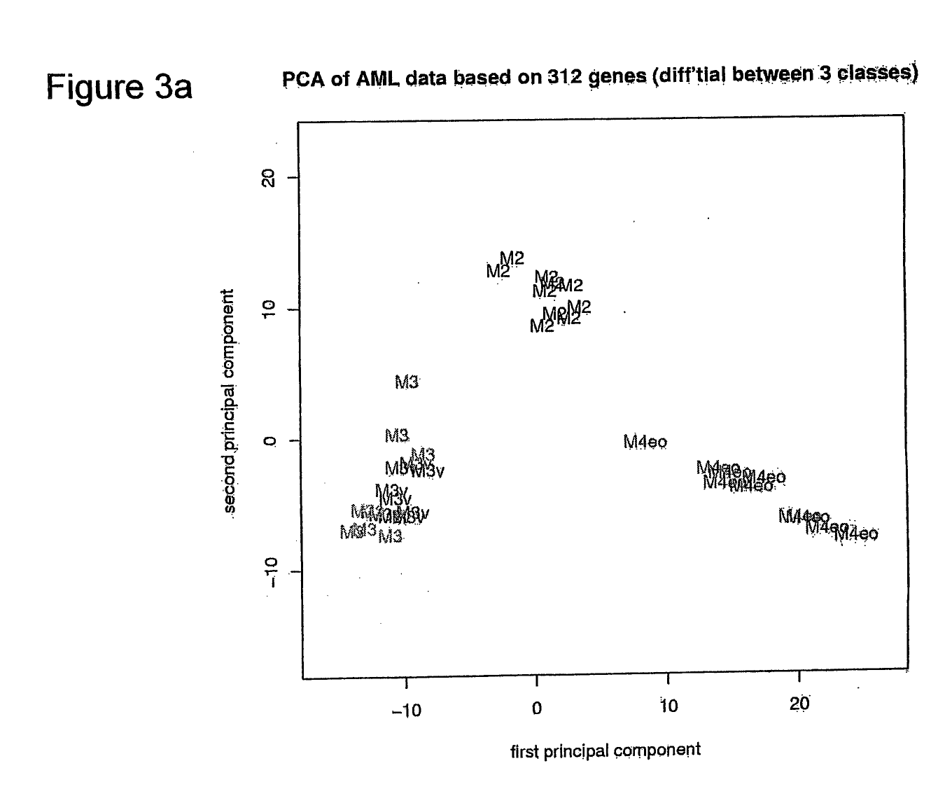

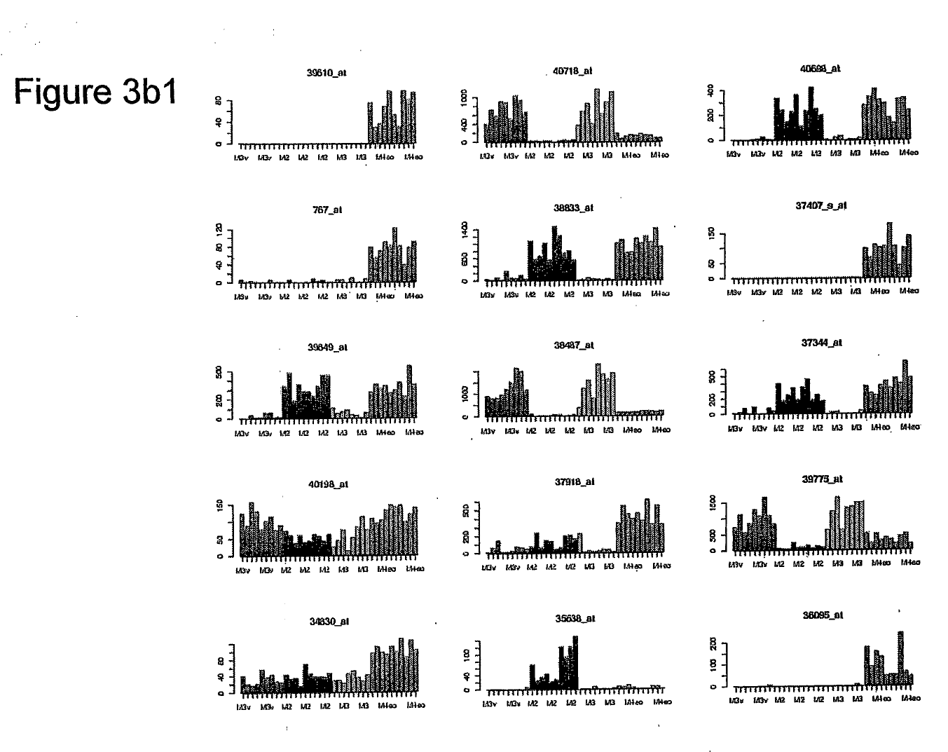

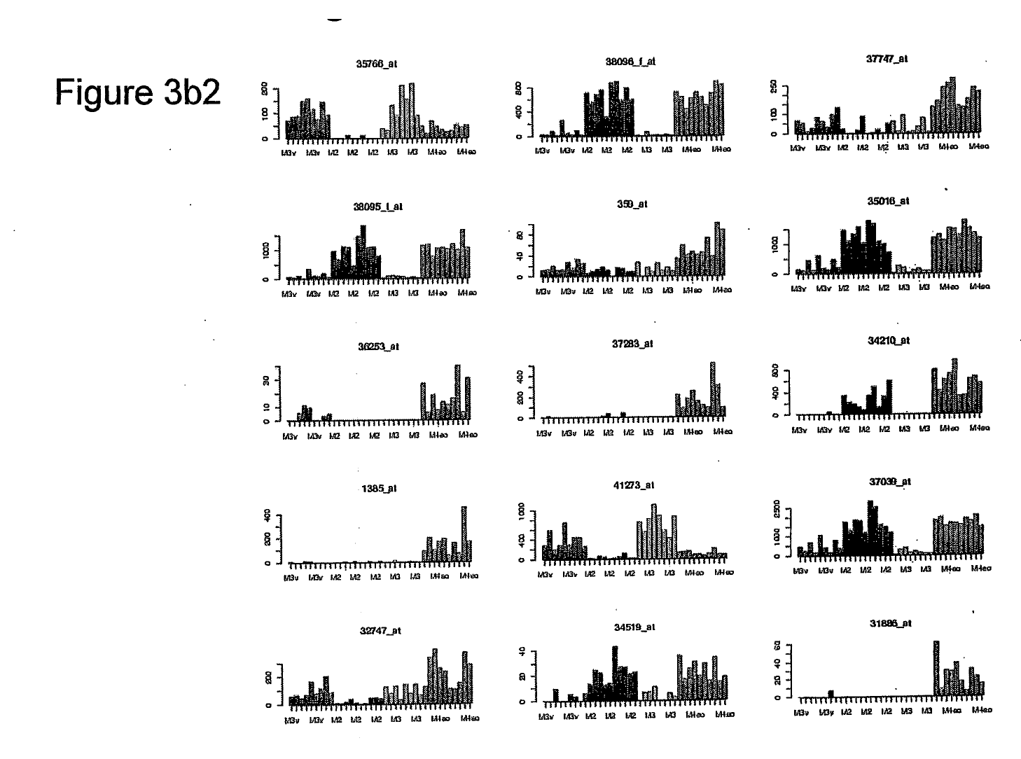

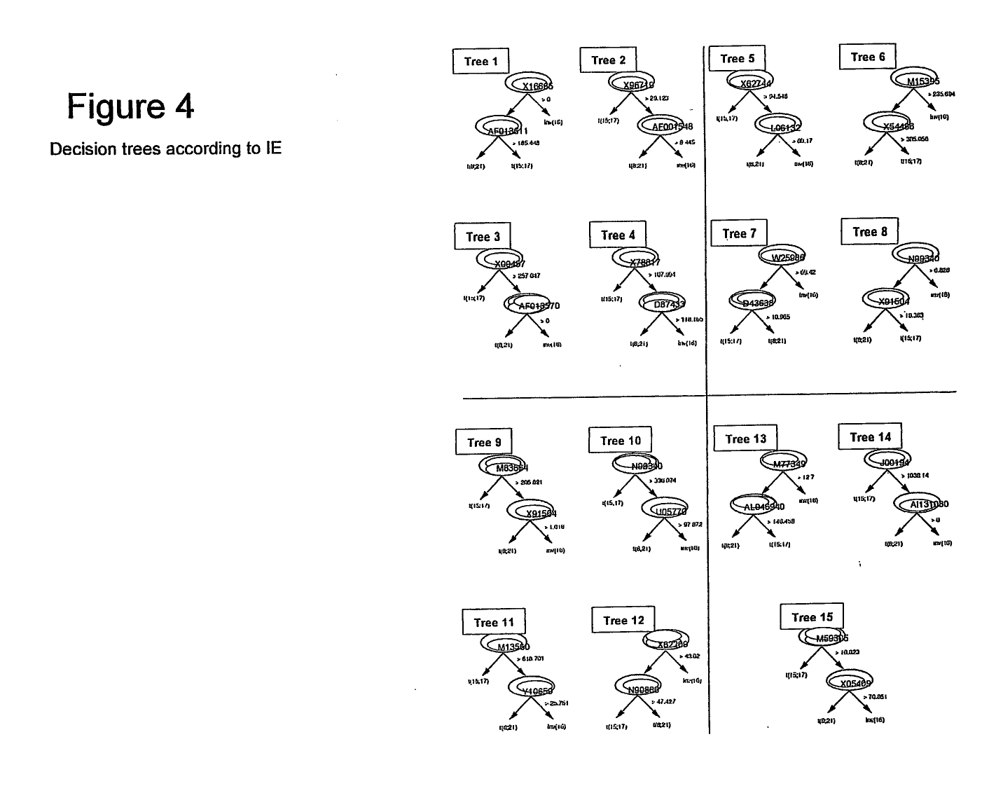

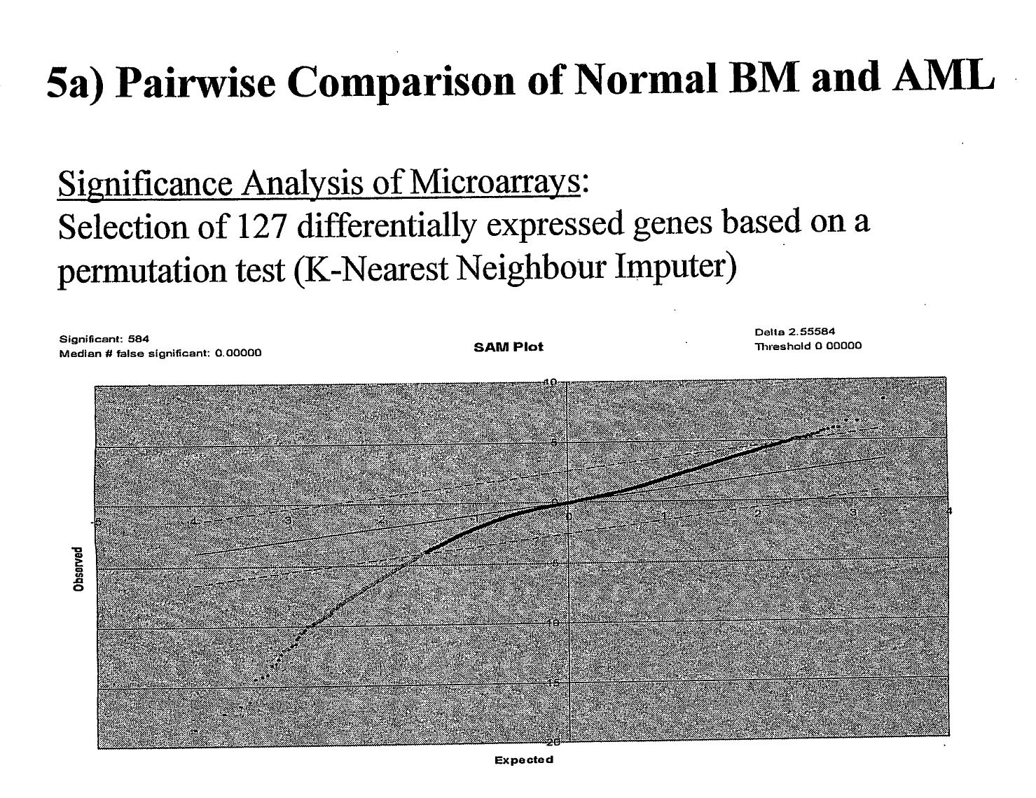

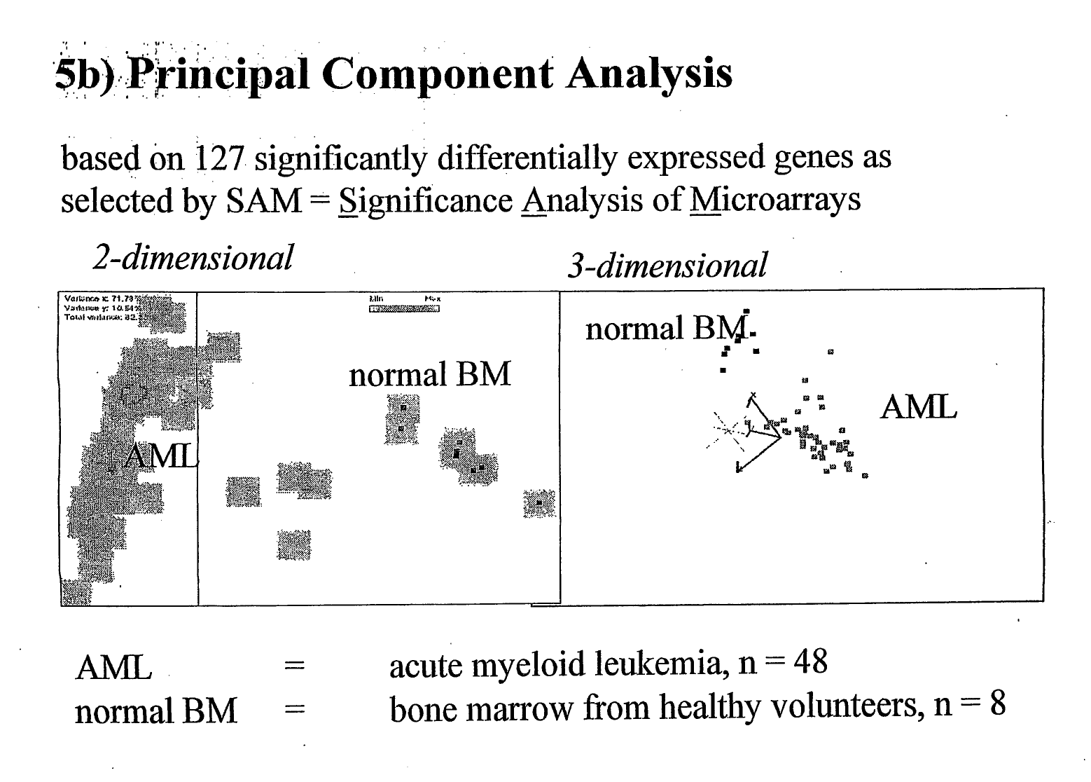

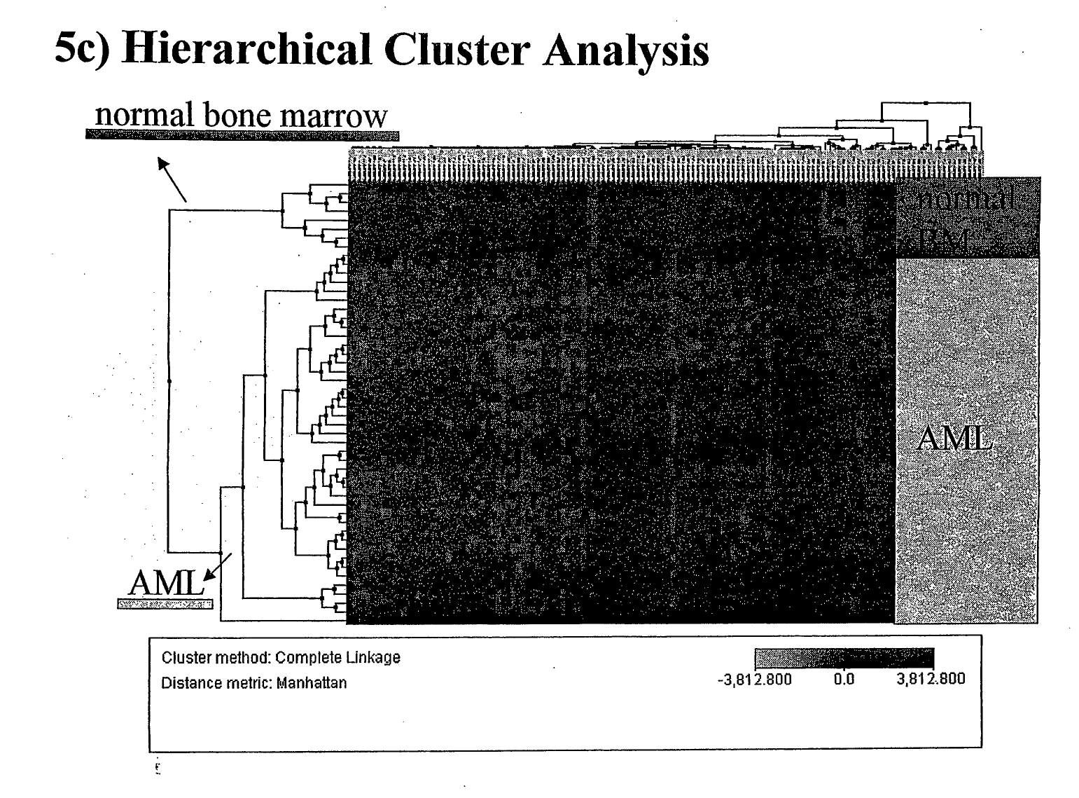

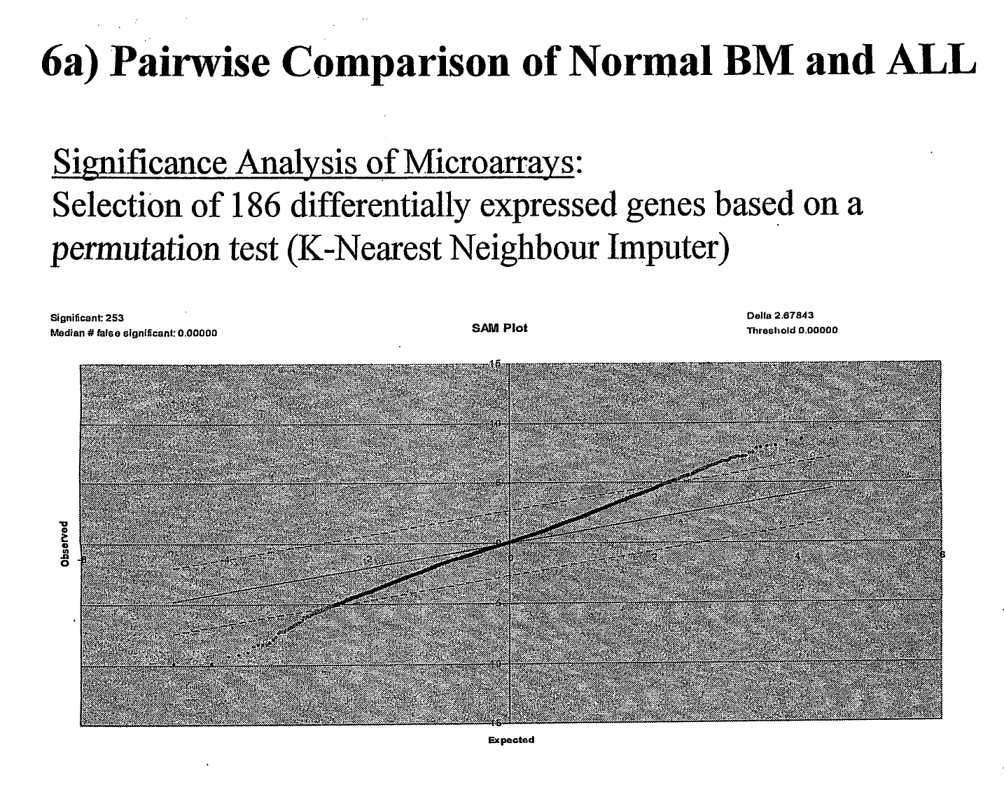

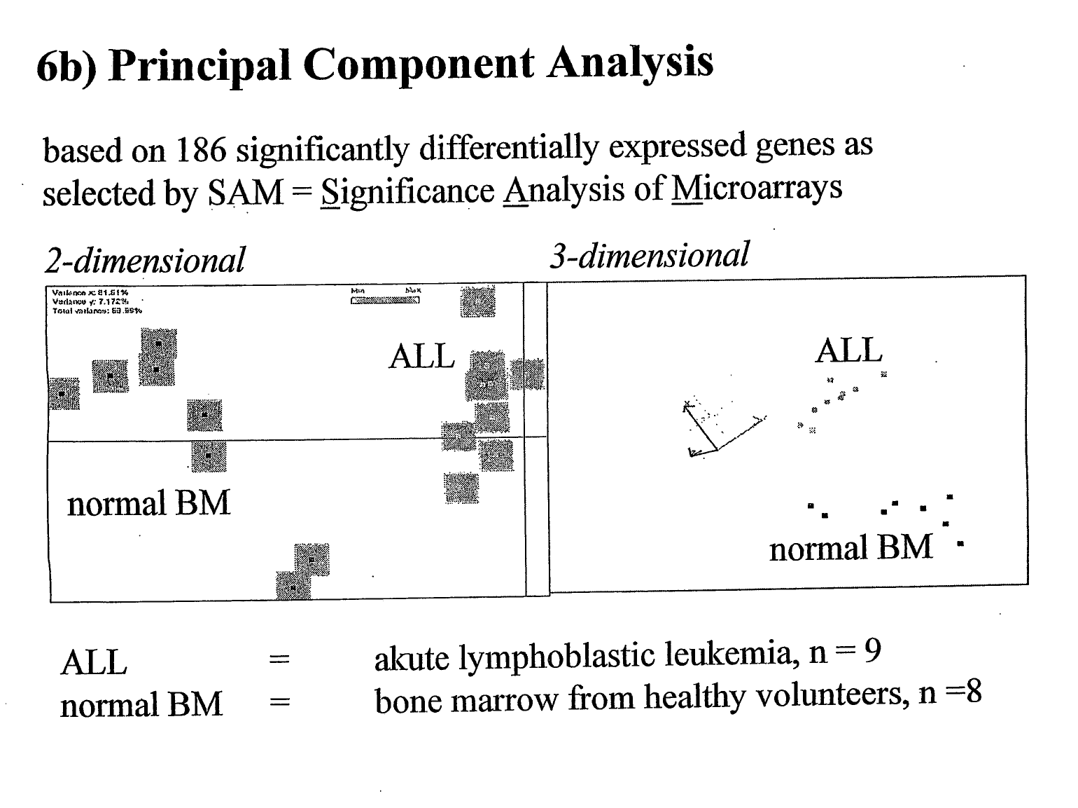

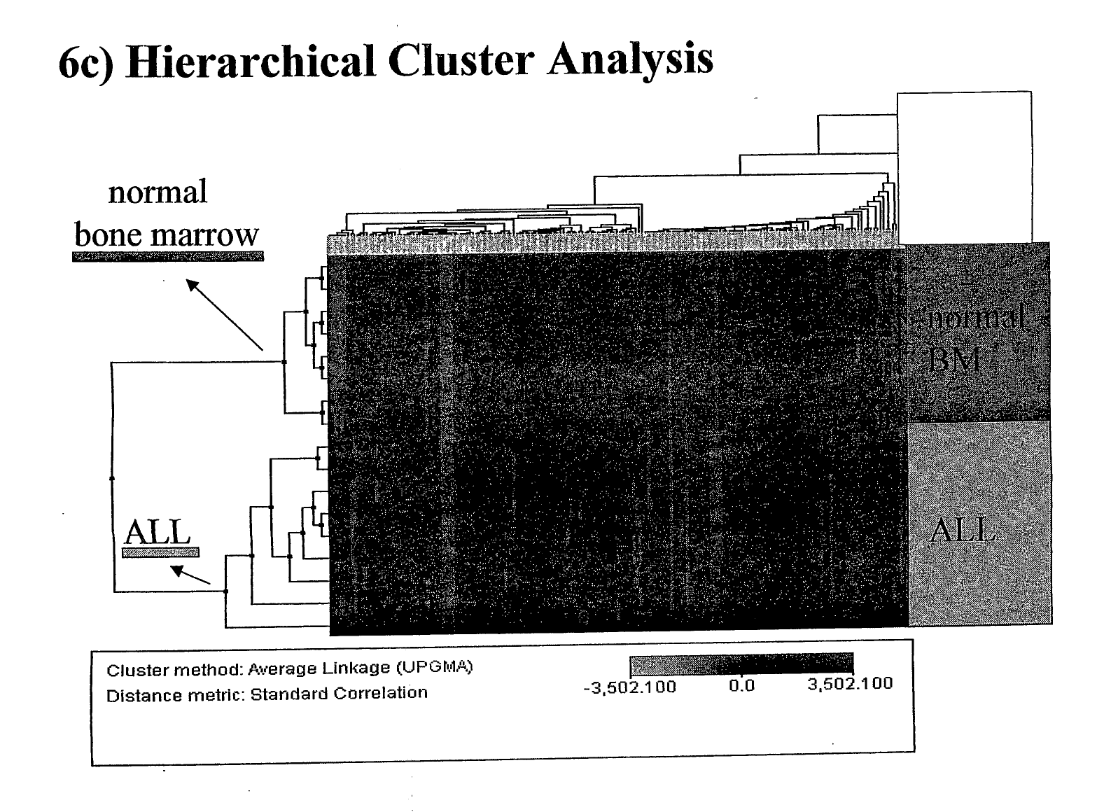

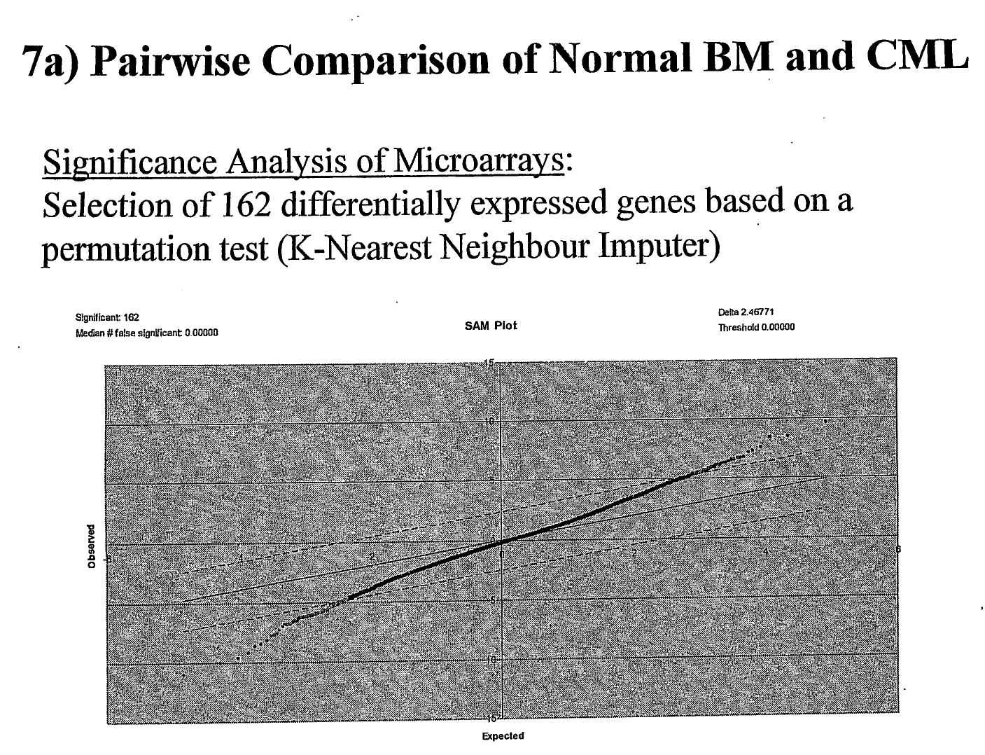

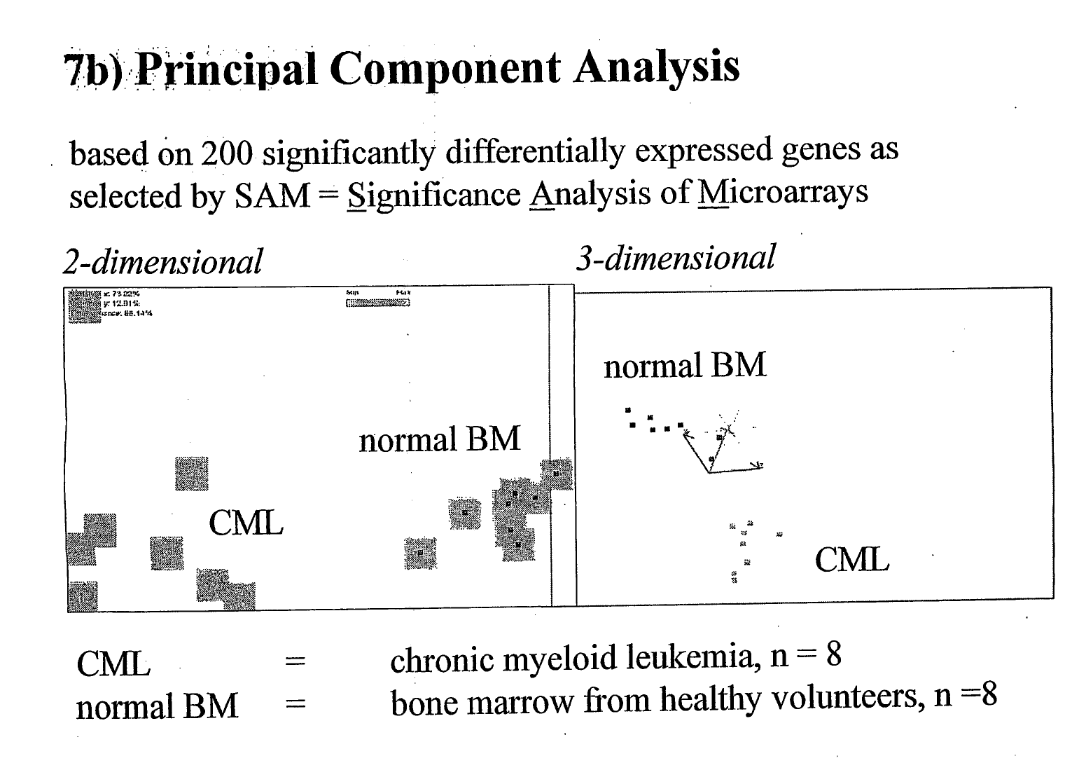

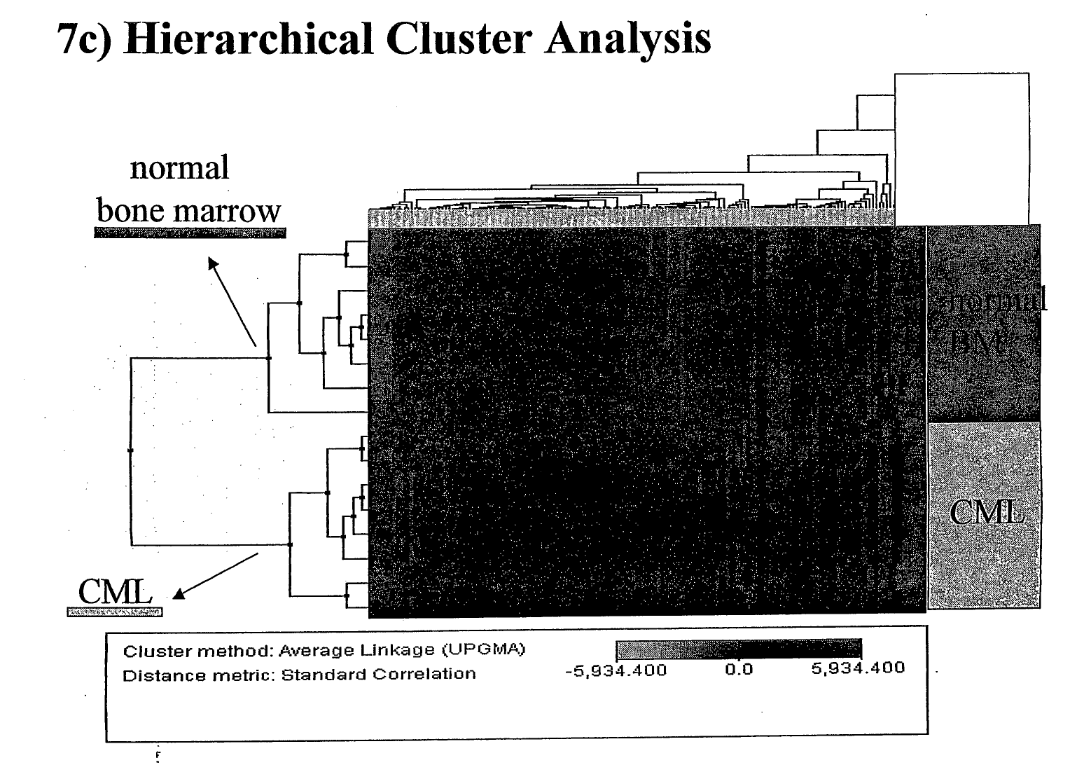

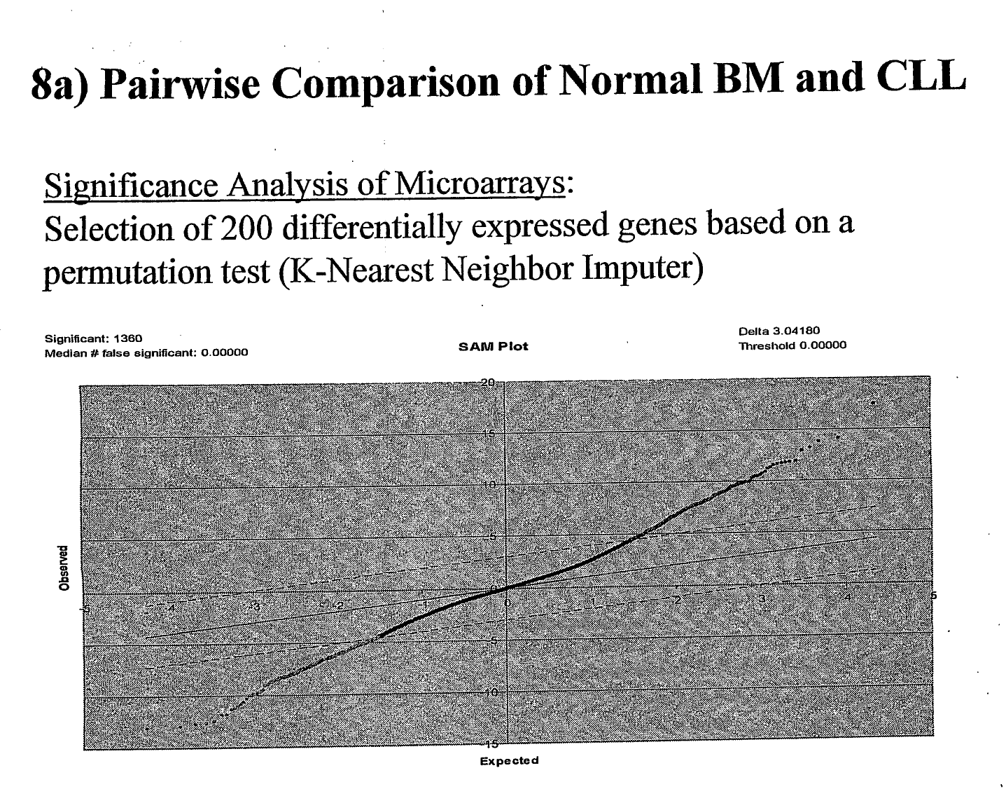

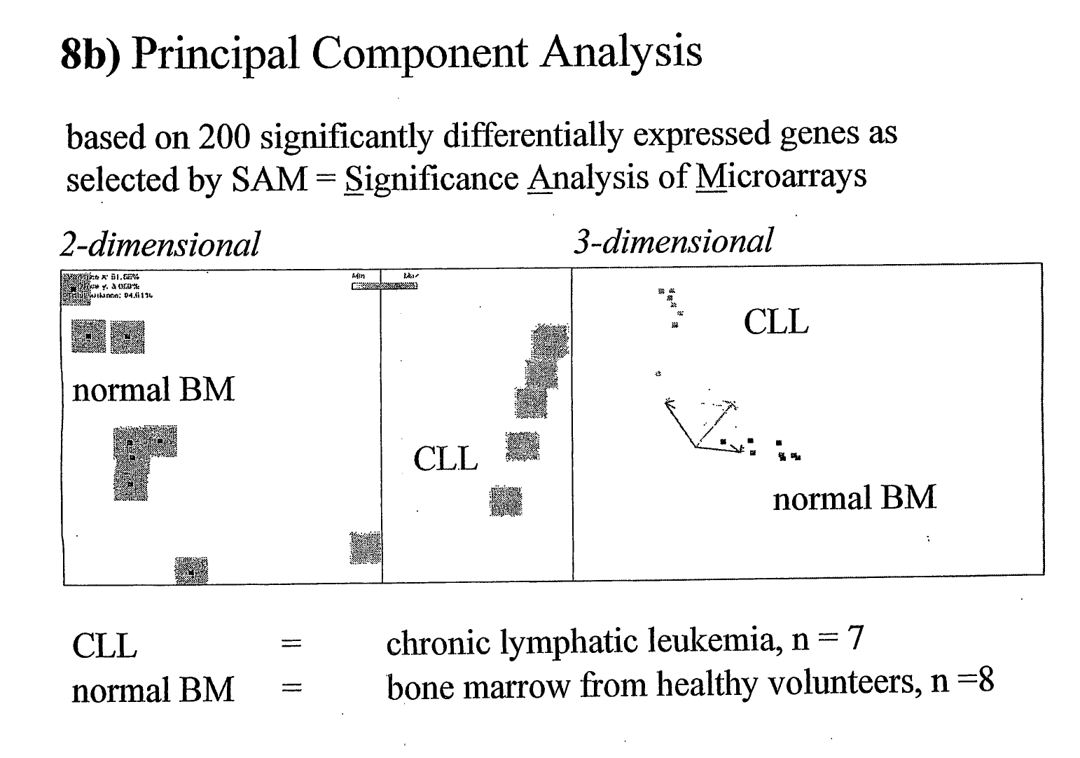

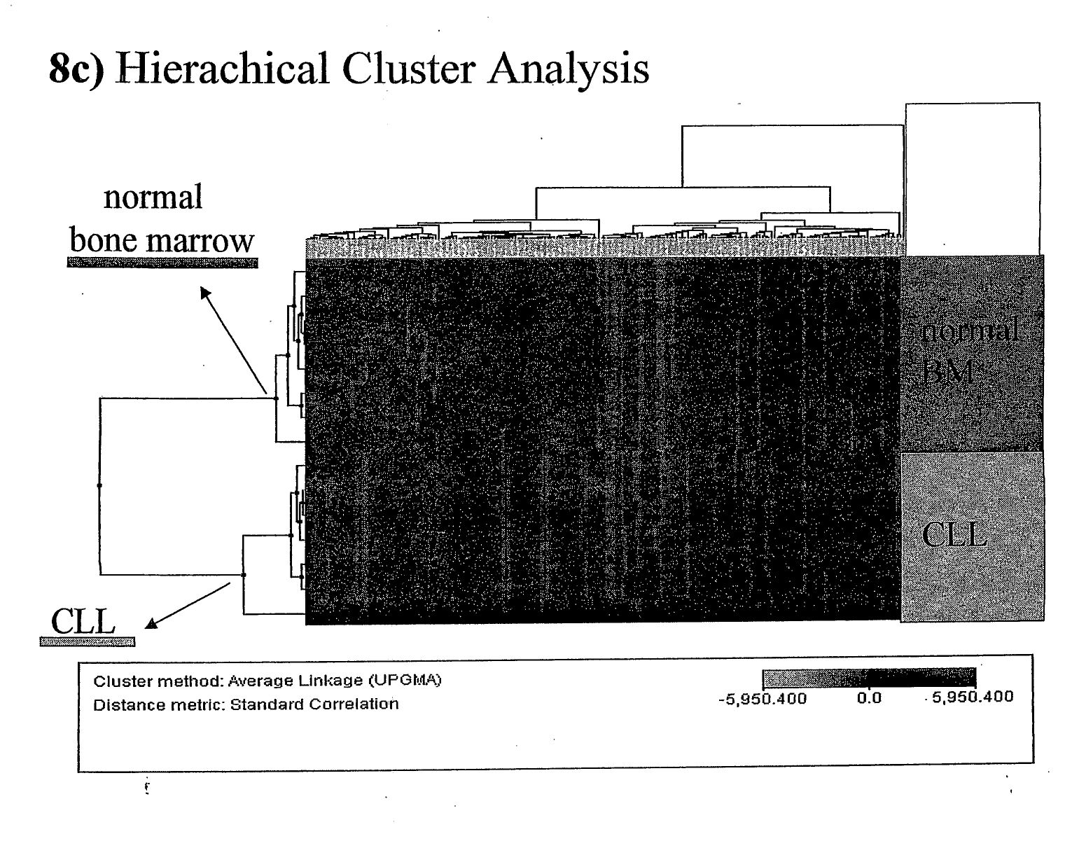

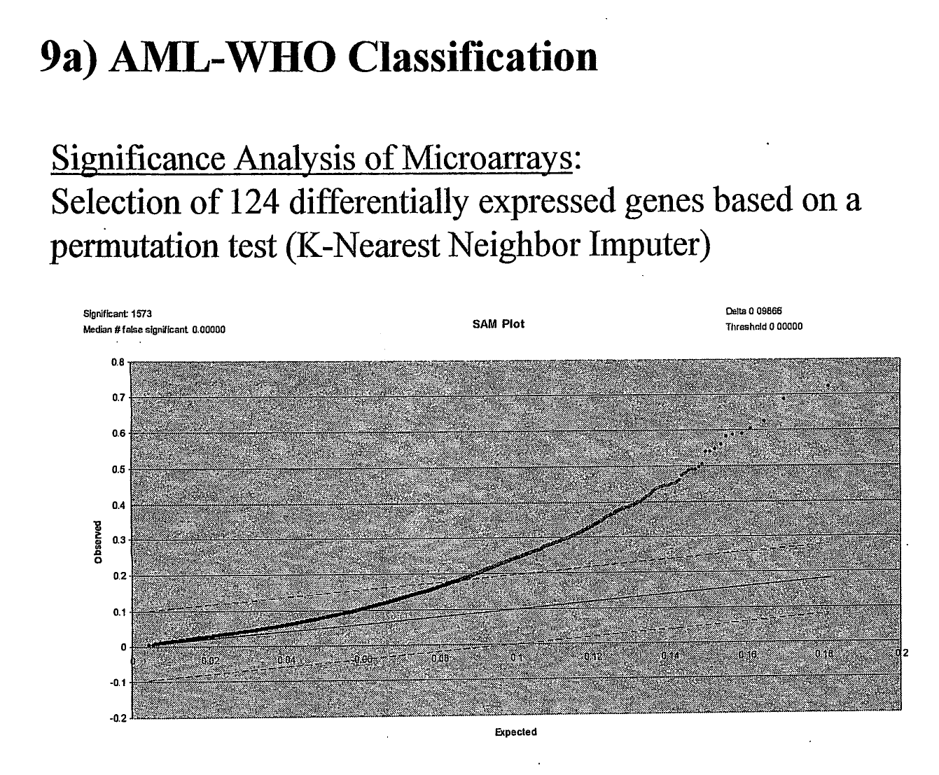

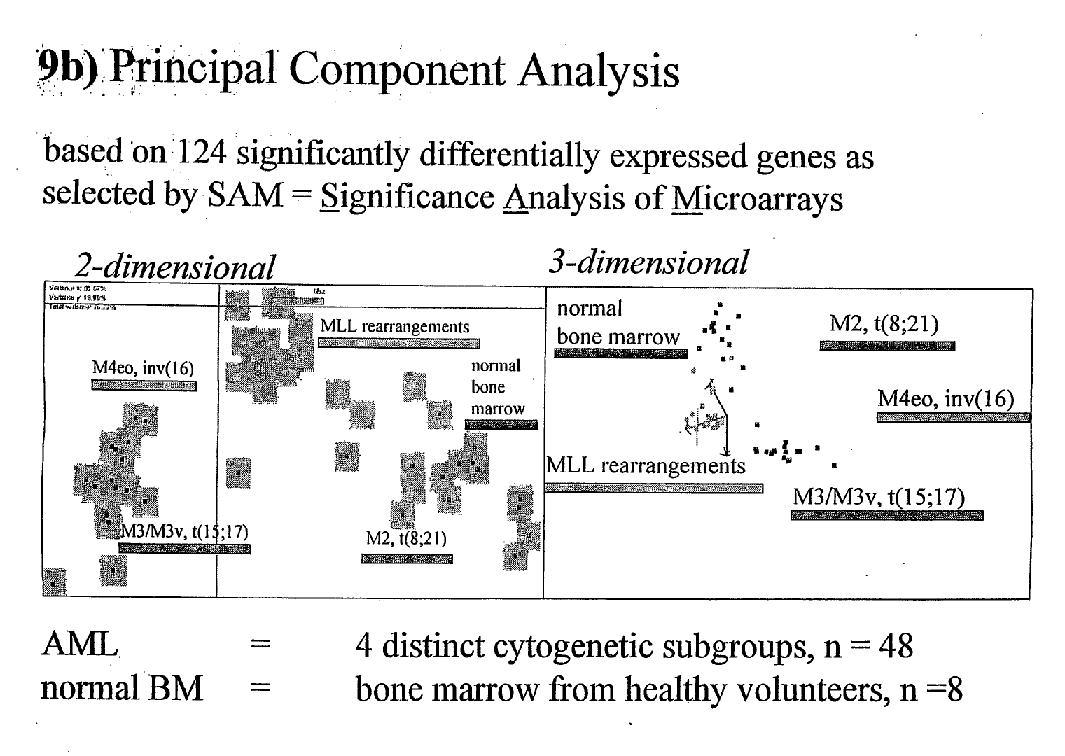

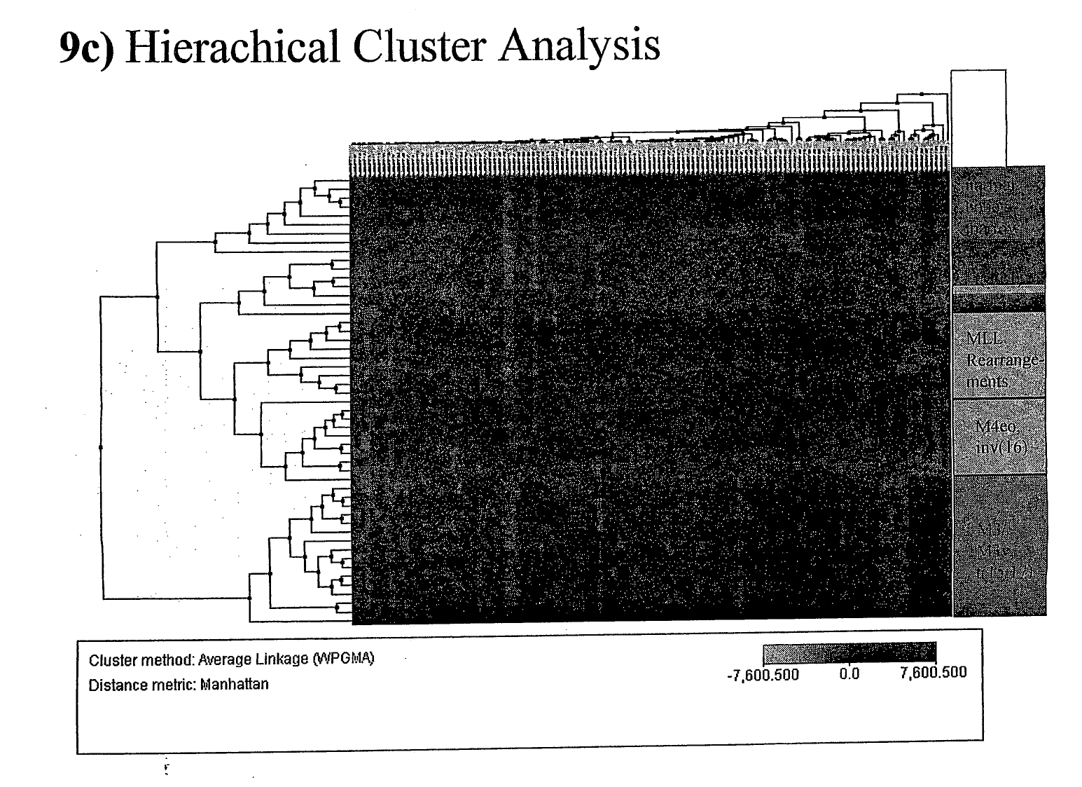

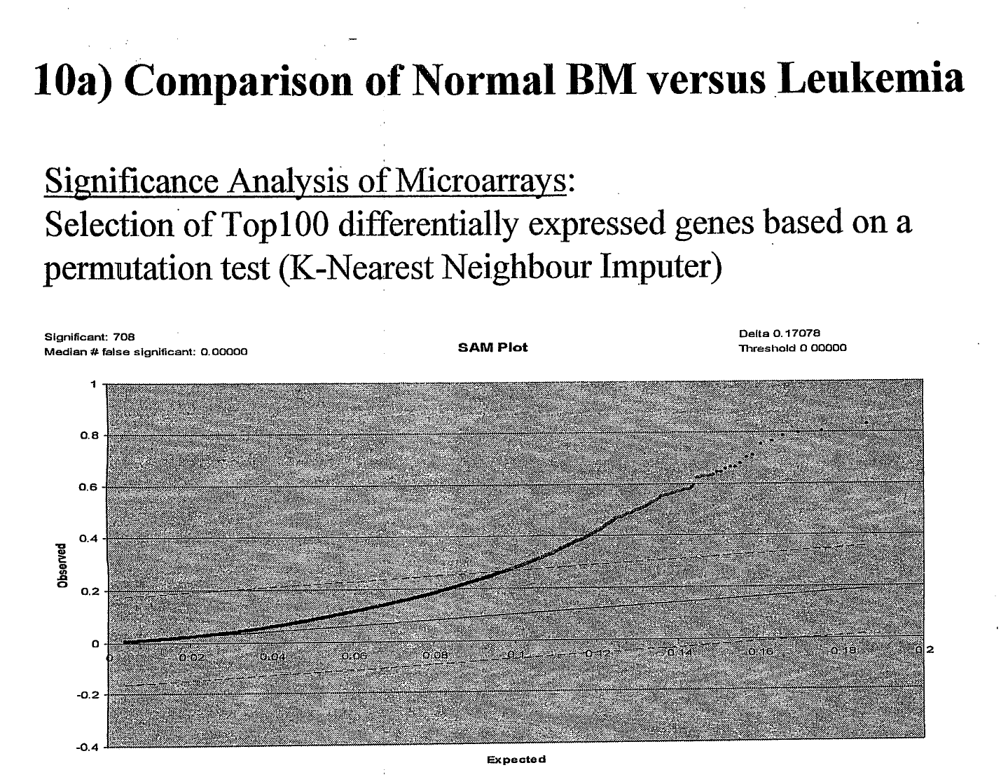

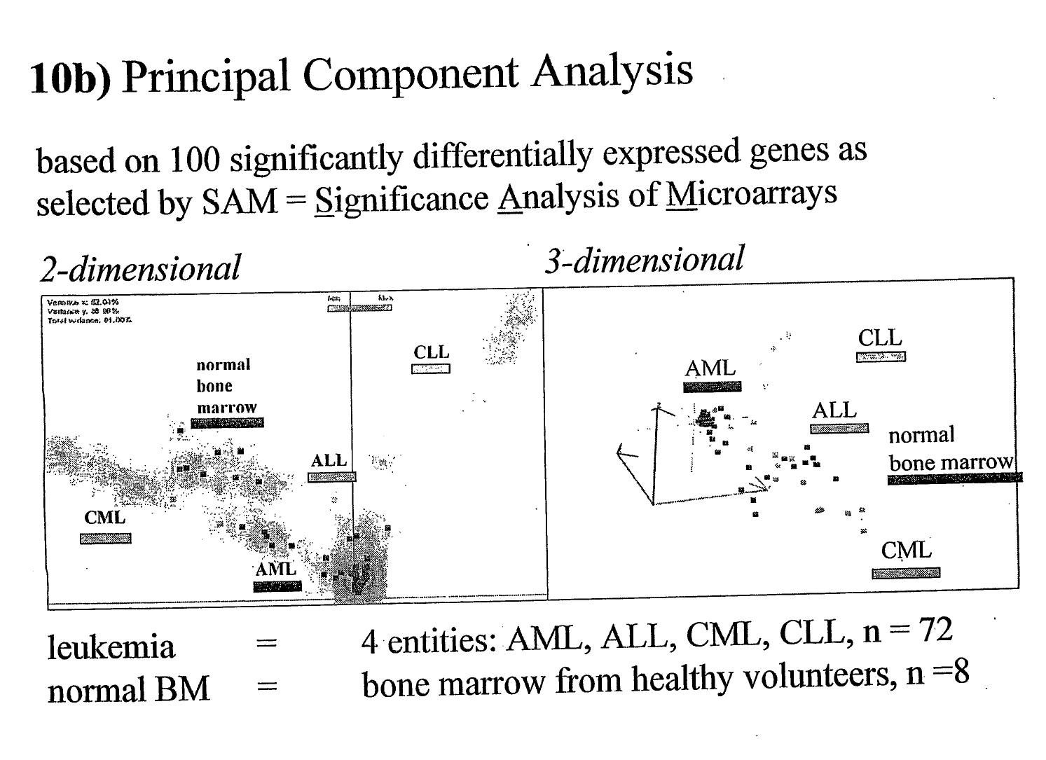

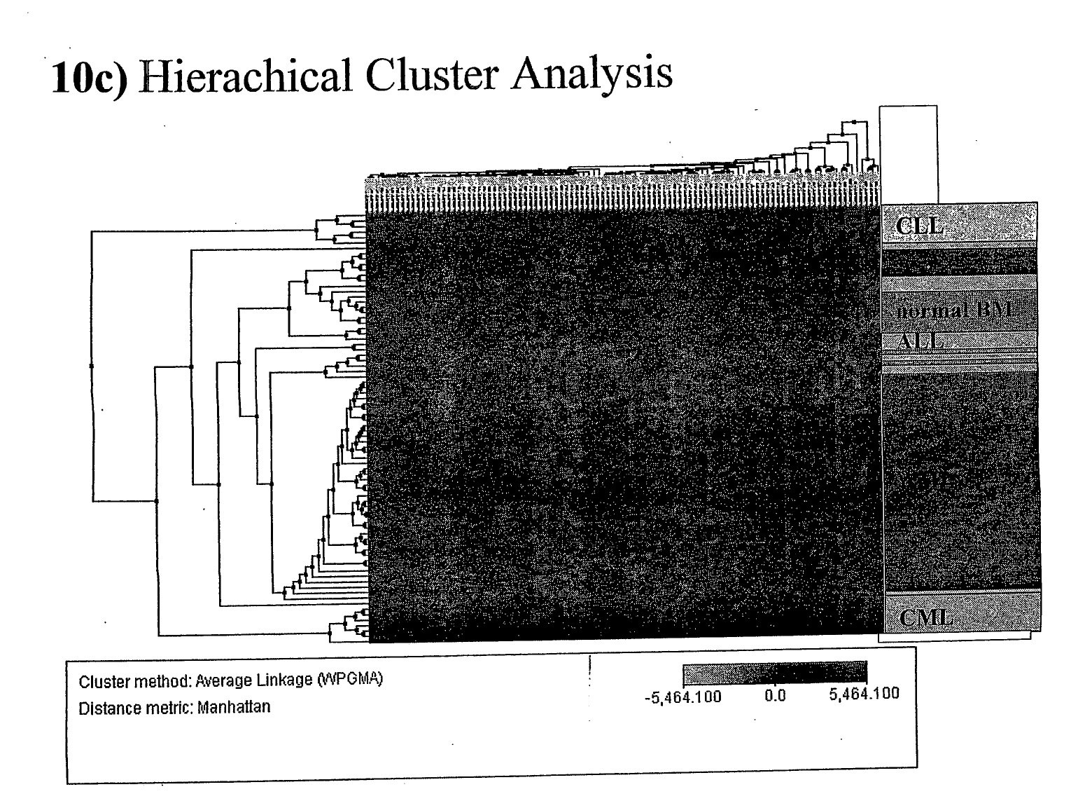

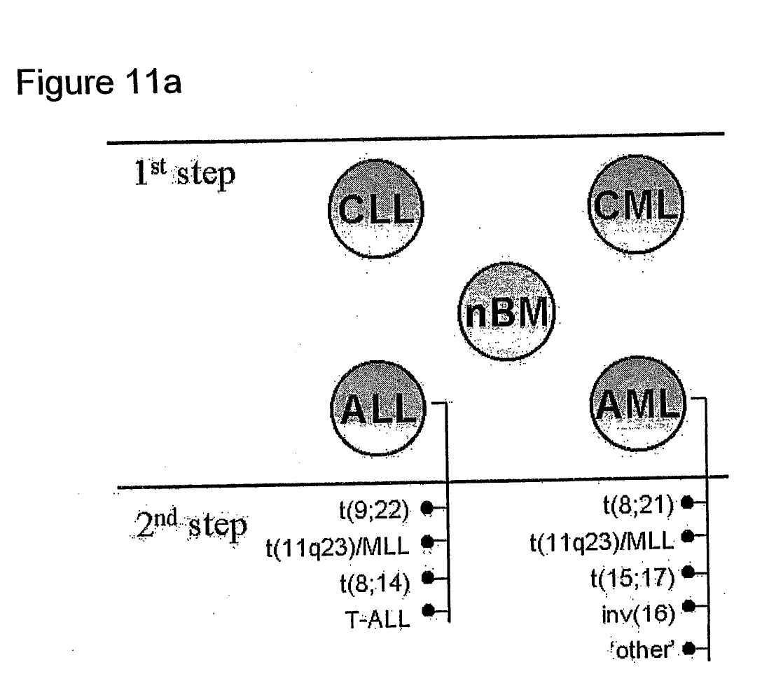

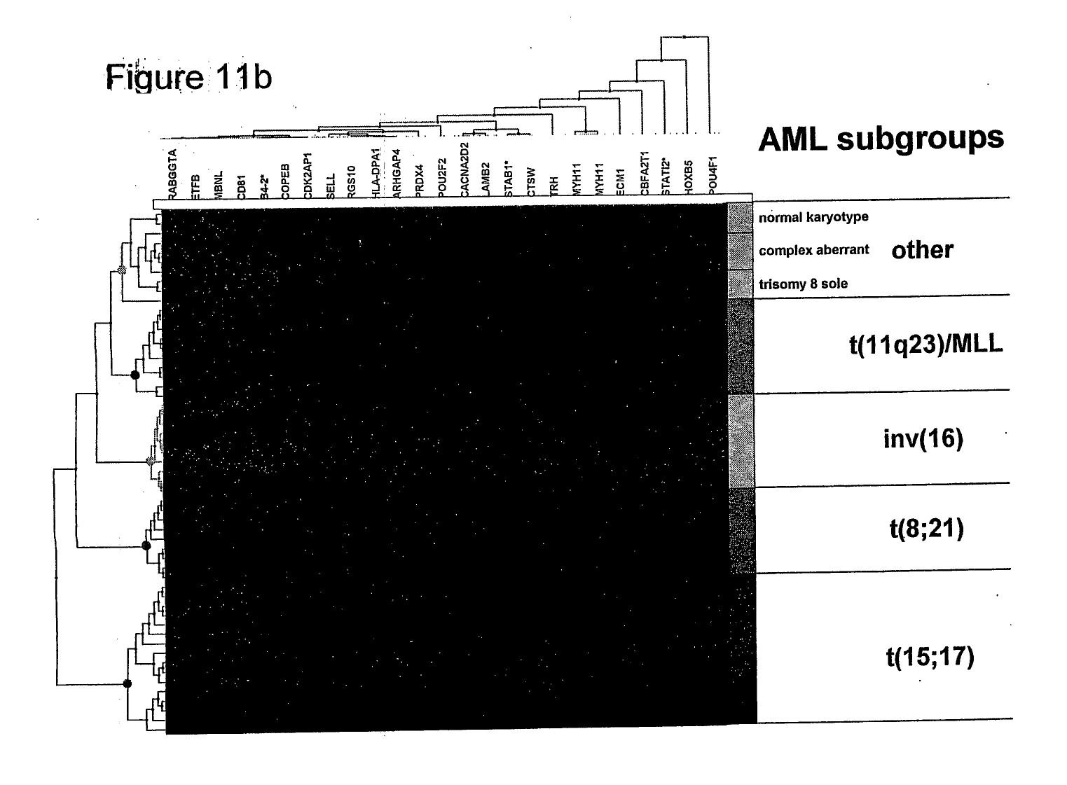

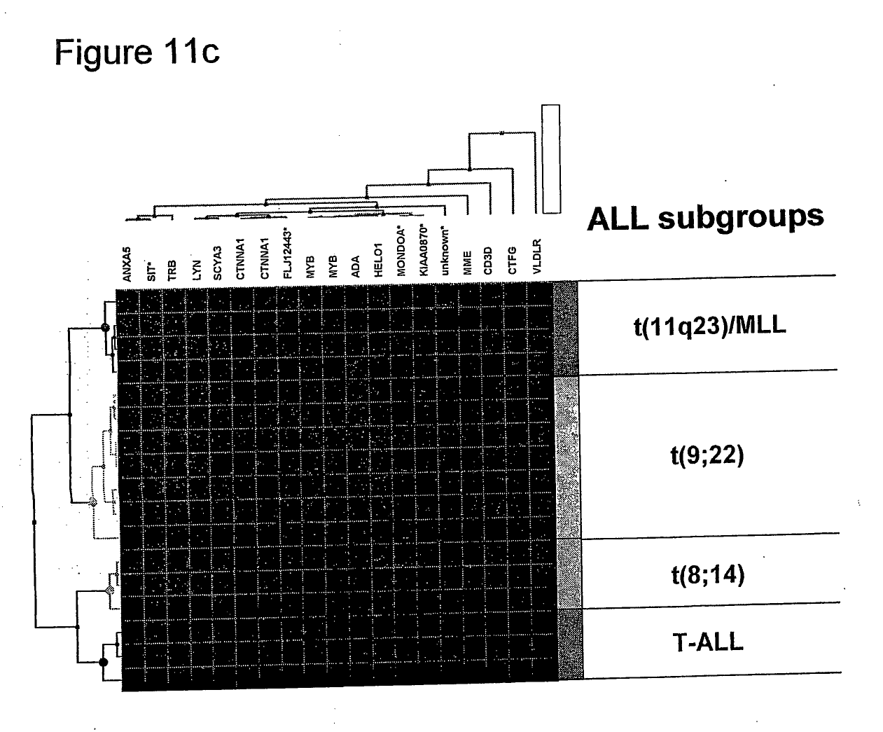

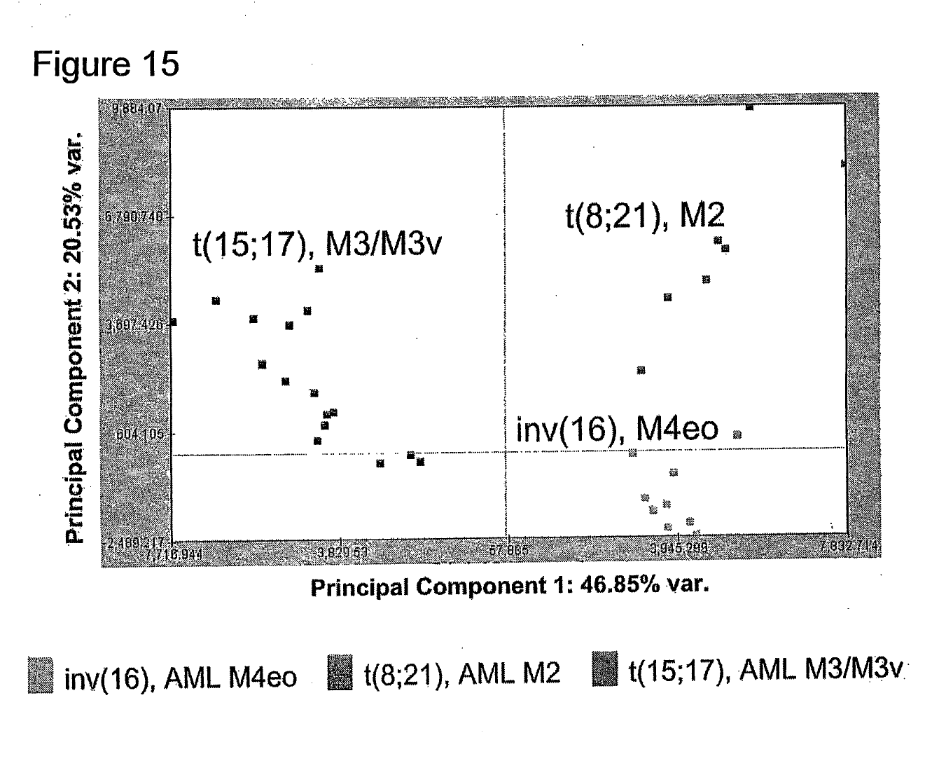

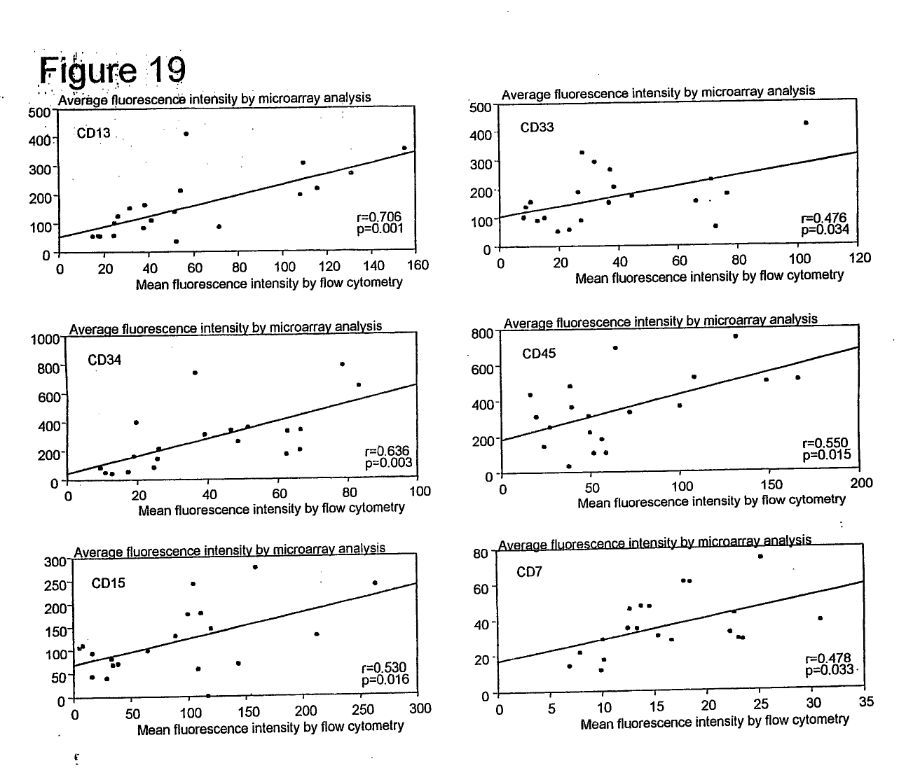

### DESCRIPTION OF THE FIGURES

| | |
|---|---|
| FIG. 1a: | Principal Component Analysis |
| FIG. 1b: | Hierarchical Cluster Analysis |
| FIG. 2: | Classification Accuracy |
| FIGS. 3a, | PCA of AML data based on 312 genes |
| 3b1, 3b2: | Decision Trees according to I(E) |
| FIG. 4: | Pair-wise Comparison of Normal BM and AML |
| FIG. 5a: | Principal Component Analysis |
| FIG. 5b: | Hierarchical Cluster Analysis |
| FIG. 5c: | Pair-wise Comparison of Normal BM and ALL |
| FIG. 6a: | Principal Component Analysis |
| FIG. 6b: | Hierarchical Cluster Analysis |
| FIG. 6c: | Pair-wise Comparison of Normal BM and CML |
| FIG. 7a: | Principal Component Analysis |
| | Hierarchical Cluster Analysis |
| FIG. 7c: | Pair-wise Comparison of Normal BM and CLL |
| FIG. 8a: | Principal Component Analysis |
| FIG. 8b: | Hierarchical Cluster Analysis |
| FIG. 8c: | AML-WHO Classification |
| FIG. 9a: | Principal Component Analysis |
| FIG. 9b: | Hierarchical Cluster Analysis |
| FIG. 9c: | Comparison of Normal BM versus Leukemia |
| FIG. 10a: | Principal Component Analysis |
| FIG. 10b: | Hierarchical Cluster Analysis |
| FIG. 10c: | |
| | Accurate diagnosis of leukemia is accomplished in a two-step |
| | approach. First, samples are assigned to one of the major |
| | leukemia types or normal BM, respectively. Then, if positive for |
| | ALL or AML, further subclassification based on cytogenetically |
| | defined characteristics is proposed. In total 111 samples were |
| | analyzed by gene expression profiling and implemented in the |
| | development of different class prediction models: normal BM |
| | (n = 8), CLL (n = 8), CML (n = 10), ALL (n = 18), and AML (n = 59). 18 |
| | ALL samples can further be characterized by B-lineage ALL |
| | samples positive for t(8; 14) (n = 3), t(9; 22) (n = 7), or t(11q23)/MLL |
| | (n = 4) and T-lineage ALL (n = 3), respectively. Additionally, one B- |
| | ALL sample showed an aberrant karyotype. 59 AML samples |
| | were comprized of normal karyotype (n = 3), complex aberrant |
| | karyotype (n = 4), trisomy 8 as sole abnormality (n = 3), t(8; 21) |
| | (n = 9), t(15; 17) (n = 16), inv(16) (n = 10), and t(11q23)/MLL (n = 10). |
| | The latter four AML entities were additionally represented by |
| | each of the following t(8; 21), +8 (n = 1), t(15; 17), +8 (n = 2), and |
| | inv(16), +8 (n = 1). Furthermore, some expression profiles were |
| | excluded for development of the classifier but subsequently |
| | tested for performance in diagnostical class assignments: normal |
| | BM (n = 1), CLL (n = 2), CML (n = 2), ALL with t(4; 11) (n = 1), and |
| | AML with t(15; 17) (n = 2), respectively. |
| FIG. 11b: | Hierarchical clustering of 55 AML samples (rows) versus 25 |
| | informative genes (columns). In total, 15 comparisons within the 5 |
| | groups were performed (pairwise and one-versus-all). Genes |
| | were selected for maximal accuracy and confidence based on a |
| | modified signal-to-noise (S2N) algorithm. The scaled gene |
| | expression levels are coded by intensity and shown on a scale |
| | from black (no expression) to bright red (highest expression). The |
| | AML subgroups ‘other’ (n = 10), t(11q23)/MLL (n = 10), inv(16) |
| | (n = 10), t(8; 21) (n = 9), and t(15; 17) (n = 16) are colored according |
| | to their chromosomal aberrations. The minimal set of informative |
| | genes is given by HGNC approved symbols (not yet approved |
| | genes are marked by asterisks). |
| | Hierarchical clustering of 17 ALL samples (rows) versus 19 |
| | informative genes (columns). In total, 10 pairwise or OVA |
| | comparisons within the 4 groups were performed. Genes were |
| | selected for maximal accuracy and confidence based on a |
| | modified S2N algorithm. The scaled gene expression levels are |
| | coded by intensity and shown on a scale from black (no |
| | expression) to bright red (highest expression). The ALL |
| | subgroups t(11q23)/MLL (n = 4), t(9; 22) (n = 7), t(8; 14) (n = 3), and |
| | T-ALL (n = 3) are colored according to their characteristical |
| | chromosomal aberrations or immunophenotype. The minimal set |
| | of informative genes is given by HGNC approved symbols |
| | (asterisks mark not yet approved genes). |
| FIG. 12a-12i | Bar graphs of gene expression intensities for distinct leukemia |
| | types and subtypes. A short description indicates the respective |
| | classes which can be distinguished at each case. |
| FIG. 13a. | Dot plot of expression levels for a particular gene in two groups |
| | (e.g. group1 = normal samples, group2 = disease samples). |
| | Golub's decision limit to distinguish between group1 and group2, |
| | which is defined as the mean of μ₁ and μ₂ (μₐ: mean expression |
| | in group a), is not optimal, because the standard deviations of |
| | gene expression levels within the two groups are very different. In |
| | this case, a lower limit (e.g. maximum level in group1) would |
| | have been more appropriate to separate the two groups by |
| | means of gene expression levels. |
| | Accuracy and confidence for all-pairs and one-versus-all |
| | comparisons in a dataset consisting of 103 samples from 5 |
| | classes (A, B, C, D, E) using Golub's method and diffgenes. Both |
| | accuracy and confidence are higher with diffgenes. |
| | Detailed characteristics of the 37 AML cases representing three |
| | defined cytogenetic aberrations corresponding to four |
| | cytomorphological subtypes according to FAB classification: |
| | inv(16)(p13q22)/AML M4eo, t(8; 21)(q22; q22)/AML M2, and |
| | t(15; 17)(q22; q12)/AML M3 or M3v. Diagnosis was proven by a) |
| | karyotype analysis, b) interphase-FISH (CBFB, AML1 and ETO, |
| | PML and RARA), c) RT-PCR (CBFB-MYH11, AML1-ETO, PML- |
| | RARA), and d) cytomorphology. |
| | FIG. 15: Three cytogenetically defined AML subtypes with |
| | t(15; 17), t(8; 21) or inv(16) can be separated based on their gene |
| | expression profiles of 1,000 preselected genes. The three |
| | different subgroups form distinct clusters. For visualization in a |
| | two-dimensional plot the first two principal components were |
| | chosen as they captured most of the variation in the original data |
| | set. The subgroups are coloured according to their chromosomal |
| | aberrations, respectively |
| | Hierarchical cluster analysis of the gene expression pattern of the |
| | set of 13 predictor genes as identified according to the adapted |
| | class prediction methodology introduced by Golub et al. The |
| | three distinct cytogenetic AML subgroups can clearly be |
| | separated based on their gene expression profiles. Each row |
| | represents a leukemia sample and each column a gene. The |
| | gene accession numbers are shown on the top. Varying |
| | expression levels are shown on a scale from black (no gene |
| | expression) to bright red (highest expression). The subgroups are |
| | coloured according to their chromosomal aberrations, |
| | respectively. |
| | Schematic representation of the 15 decision trees (a to o) used in |
| | the multiple-tree classifier. Arrows indicate high (arrow up) or low |
| | (arrow down) expression, “0” and “+” denote absence or |
| | presence of a gene, respectively (e.g., in (a) the low expression |
| | of X96719 indicates AML with t(15; 17) whereas the high |
| | expression of X96719 indicates AML with inv(16) or AML with |
| | t(8; 21); the latter two entities are distinguished by X53742: lack of |
| | expression identifies AML with inv(16) and positive expression |
| | predicts AML with t(8; 21)). The GenBank accession numbers are |
| | given for genes the expression level of which are used for |
| | decision. Nodes are represented as ovals and leaves as |
| | rectangles. Classes are referred to as t(15; 17), t(8; 21) or inv(16). |
| | Based on a preselection of 82 genes morphologically different but |
| | cytogenetically identical AML subtypes M3 with t(15; 17) and M3v |
| | with t(15; 17) can be separated based on their gene expression |
| | profile. AML M3 samples are shown as green dots, AML M3v |
| | samples as blue dots, respectively. |
| FIG. 19: | Correlations between protein expression levels and mRNA |
| | abundance. Expression levels were compared by Pearson's |
| | correlation. Mean fluorescence intensity values obtained by flow |
| | cytometry were calculated for all events with fluorescence values |
| | higher than isotype controls using the CellQuest Pro software |
| | (Beckton Dickinson). Average fluorescence intensity values |
| | obtained by micorarray analyses were calculated by the |
| | Affymetrix software, Microarray Suite, Version 4.0.1. |
| | Detailed characteristics of the 45 AML cases representing four |
| | defined recurrent cytogenetic abnormalities. Diagnosis was |
| | proven by a) karyo-type analysis, b) interphase-FISH, c) RT- |
| | PCR, and d) cytomorphology. |
| | Class separation by principal component analysis (PCA) |
| | FIG. 3: PCA-Plot based on 39 informative genes. All leukemia |
| | samples could accurately be assigned to their corresponding |
| | cytogenetic subtype with 100% accuracies. To illustrate these |
| | results, a hierarchical clustering is shown (FIG. 4). |
| | Hierarchical clustering of 44 diagnostic AML samples and 8 |
| | normal BM samples (columns) versus 39 informative genes |
| | (rows). Gene expression levels are coded by intensity and |
| | represented on a scale from black (no expression) to bright red |
| | (highest expression). |
| FIG. 24 to 465 | Bar graphs of gene expression intensities for distinct leukemia |
| | types and subtypes or normal bone marrow, respectively. |
| | Selected statistically significant genes are given by Affymetrix |
| | identification number and Human Gene Nomenclature Committee |
| | approved symbol (where available). A short description indicates |
| | the respective classes which can be distinguished at each case. |

### LIST OF REFERENCES

Breiman et al., Classification and regression try, Wadsworth & Brooks/Cole (Monterey)
Efron and Tibshirani, An introduction to the bootstrap (1993), Chapman & Hall (New York, London), pp. 237-247
Jolliffe, Principle Components Analysis (1986), Springer (New York)
Lockhart, D. J., et al., Nat Biotechnol 14 (1996) 1675-80
Sambrook, J., et al., in “Molecular Cloning: A Laboratory Manual” (1989), Eds. J. Sambrook, E. F. Fritsch and T. Maniatis, Cold Spring Harbour Laboratory Press, Cold Spring Harbour, NY
EP 0 237 362

The following examples, references, sequence listing and figures are provided to aid the understanding of the present invention, the true scope of which is set forth in the appended claims. It is understood that modifications can be made in the procedures set forth without departing from the spirit of the invention.

### EXAMPLES

### Example 1

### Example 1

### General Methods

### Example 1

### (A) Selection and Characterisation of Leukemia Samples

Bone marrow (BM) aspirates were taken at the time of the initial diagnostic biopsy and remaining material was immediately lysed in RLT buffer (Qiagen), frozen and stored at −80 C until preparation for gene expression analysis. For microarray analysis the GeneChip System (Affymetrix, Santa Clara, Calif., USA) was used. The targets for GeneChip analysis were prepared according to the current Expression Analysis. Briefly, frozen lysates of the leukemia samples were thawed, homogenized (QIAshredder, Qiagen) and total RNA extracted (RNeasy Mini Kit, Qiagen). Normally 10 ug total RNA isolated from 1×107 cells was used as starting material in the subsequent cDNA-Synthesis using Oligo-dT-T7-Promotor Primer (cDNA synthesis Kit, Roche Molecular Biochemicals). The cDNA was purified by phenol-chlorophorm extraction and precipitated with 100% Ethanol over night. For detection of the hybridized target nucleic acid biotin-labeled ribonucleotides were incorporated during the in vitro transcription reaction (Enzo® BioArray™ HighYield™ RNA Transcript Labeling Kit, ENZO). After quantification of the purified cRNA (RNeasy Mini Kit, Qiagen), 15 ug were fragmented by alkaline treatment (200 mM Tris-acetate, pH 8.2, 500 mM potassium acetate, 150 mM magnesium acetate) and added to the hybridization cocktail sufficient for 5 hybridizations on standard GeneChip microarrays. Before expression profiling Test3 Probe Arrays (Affymetrix) were chosen for monitoring of the integrity of the cRNA. Only labeled cRNA-cocktails which showed a ratio of the measured intensity of the 3′ to the 5′ end of the GAPDH gene less than 3.0 were selected for subsequent hybridization on HG-U95Av2 probe arrays (Affymetrix). Washing and staining the Probe arrays was performed as described (siehe Affymetrix-Original-Literatur (LOCKHART und LIPSHUTZ). The Affymetrix software (Microarray Suite, Version 4.0.1) extracted fluorescence intensities from each element on the arrays as detected by confocal laser scanning according to the manufacturers recommendations.

### Example 1

### (B) Data Analysis

Class separation by principal component analysis and hierarchical cluster analysis: In a first step we reduced the dimensionality of the number of genes. Therefore we scaled the data from each array to a target intensity value 50 (Affymetrix Microarray Suite) in order to be able to perform inter-array comparisons. Then all data was analyzed using Significance Analysis of Microarrays (Multiclass Response, Stanford University) and we selected a distinct number of genes based on a permutations test. This reduced set of genes which showed to be significant then was analyzed using the public available Java application J-Express analysis tool (download at www.molmine.com). Principal Component Analysis and Hierarchical Cluster Analysis (parameters Cluster method: single linkage and Distance metric: euclidean) showed a clear separation of analyzed groups of samples e.g. healthy bone marrow versus leukemia.

### Example 1

### (C) Identification of Differentially Expressed Genes According to Golub et al. (Science 1999 Oct. 15; 286(5439):531-7)

A previously described (Science 1999 Oct. 15; 286(5439):531-7) was modified to reduce the number of candidate genes that could distinguish between our leukemic samples of interest. In a first step the raw data was scaled using Affymetrix software (target intensity 50 for all genes). To avoid division by zero or negative numbers as occurring due to the current expression algorithm (Affymetrix) we set all average intensities of 20 or less to 20. Briefly, for a more detailed gene expression profiling we applied the data analysis method according to Golub et al. using weighted voting. In a first step gene expression levels were log-transformed with a cut-off value set at 20 units. To assess the significance of selected genes we performed a leave-one-out cross-validation. Only those genes were considered important which were contained in all cross validation classificators. To determine the association between genes by chance we performed a permutation test (100 cycles). Because the number of informative genes, which are able to discriminate between samples, is unknown, we applied the Golub method for different numbers of informative genes (range: 10-200). The minimal set of genes which provided optimal classification accuracy was selected to avoid overfitting.

### Example 2

### Example 2

### Identification of Genes, the Aberrant Expression of which is Associated with a Particular Leukemia Subtype

Monitoring the gene expression level of thousands of mRNA transcripts simultaneously in one experiment is the key technology to find out the specific genes which allow the subsequent development of a class prediction model. We therefore used the Affymetrix oligonucleotide microarray technology (GeneChip® Instrument System) to obtain gene expression profiles of each individual clinical sample of interest. The HG-U95Av2 probe arrays gave us information about the relative mRNA abundance of about 12,000 full length human genes which are represented on these high-density oligonucleotide microarrays.

In total, 8 bone marrow samples of healthy volunteers and leukemia patients were investigated. Five different types of bioinformatic calculations were performed.

### Example 2

### (I) Three Distinct Genetic Subtypes of AML

Three defined cytogenetic aberrations t(8;21)(q22;q22) (n=9), t(15;17)(q22;q12) (n=16) and M4eo with inv(16) (p13q22) (n=10) corresponding to the 4 FAB-subtypes AML M2, M3, or M3v and M4eo, respectively. After we obtained bone marrow aspirates from 35 untreated patients with newly diagnosed AML, all cases were characterized by cytomorphology, cytogenetics and by molecular genetics (FIG. 1). AML subtypes M3 and M3v both carry the same chromosomal aberration but differ in morphological aspects like nuclear configuration, granulation and clinical aspects white blood cell count (WBC), respectively. In all cases, these balanced abnormalities were confirmed by fluorescence in-situ hybridization. The corresponding fusion transcript was detected by RT-PCR and/or quantitative real time PCR. The median age of the patients was 53 years (range, 19-82 years) and did not differ between the respective groups. The median WBC count was 17.0 G/I (range, 0.8-168.0 G/I) and was strikingly lower in patients with AML M3 as compared to all other patients.

### Example 2

### Methods Used

### Example 2

### (A) Selection and Characterisation of Leukemia Samples

We obtained bone marrow (BM) aspirates from 37 AML patients standing for four morphological and three underlying cytogenetic subgroups that were sent to the Laboratory of Leukemia Diagnostics (LFL) for central diagnosis within the German AMLCG study (Klinikum Grosshadern, Munich, Germany). They were selected for this study on the basis of several criteria. It was mandatory that none of the patients had been treated. All samples, exclusively newly diagnosed in our laboratory, had to be well characterized as de novo AML and diagnosis had been proven by cytomorphology, cytogenetics, flow cytometry and molecular genetics in every single case. All samples for gene expression analysis were taken at the time of the initial diagnostic biopsy when remaining material was immediately lysed in RLT buffer (Qiagen), frozen and stored at −80 C until preparation for gene expression analysis.

### Example 2

### (B) Microarray Experiments

For microarray analysis the GeneChip System (Affymetrix, Santa Clara, Calif., USA) was used. The targets for GeneChip analysis were prepared according to the current Expression Analysis Technical Manual. Briefly, frozen lysates of the leukemia samples were thawed, homogenized (QIAshredder, Qiagen) and total RNA extracted (RNeasy Mini Kit, Qiagen). Normally 10 ug total RNA isolated from 1×107 cells was used as starting material in the subsequent cDNA-Synthesis using Oligo-dT-T7-Promotor Primer (cDNA synthesis Kit, Roche Molecular Biochemicals). The cDNA was purified by phenol-chlorophorm extraction and precipitated with 100% Ethanol over night. For detection of the hybridized target nucleic acid biotin-labeled ribonucleotides were incorporated during the in vitro transcription reaction (Enzo® BioArray™ HighYield™ RNA Transcript Labeling Kit, ENZO). After quantification of the purified cRNA (RNeasy Mini Kit, Qiagen), 15 ug were fragmented by alkaline treatment (200 mM Tris-acetate, pH 8.2, 500 mM potassium acetate, 150 mM magnesium acetate) and added to the hybridization cocktail sufficient for 5 hybridizations on standard GeneChip microarrays. Before expression profiling Test3 Probe Arrays (Affymetrix) were chosen for monitoring of the integrity of the cRNA. Only labeled cRNA-cocktails which showed a ratio of the measured intensity of the 3′ to the 5′ end of the GAPDH gene less than 3 were selected for hybridization on HG-U95Av2 probe arrays (Affymetrix). Washing and staining the Probe arrays was performed as described. The Affymetrix software (Microarray Suite, Version 4.0.1) extracted fluorescence Intensities from each element on the arrays as detected by confocal laser scanning according to the manufacturers recommendations.

### Example 2

### (C) Class Separation by Principal Component Analysis and Hierarchical Cluster Analysis

In a first step we reduced the dimensionality of the number of genes. Therefore we scaled the data from each array to a target intensity value 50 (Affymetrix Microarray Suite) in order to be able to perform inter-array comparisons. Then all data was analyzed using Significance Analysis of Microarrays (Multiclass Response, Stanford University) and we selected 580 genes based on a permutations test. This reduced set of genes which showed to be significant then was analyzed using the public available Java application J-Express analysis tool (download at www.molmine.com). Principal Component Analysis and Hierarchical Cluster Analysis (parameters Cluster method: single linkage and Distance metric: euclidean) showed a clear-separation-of-analyzed groups-of-samples e.g. healthy bone marrow versus leukemia.

### Example 2

### (D) Identification of Differentially Expressed Genes According to Golub

This analysis was carried cut as described in Example 1 (C) above. Briefly, classification of tumor samples was achieved by using a set of samples whose class had been already determined. This set was called training set. By using the oligonucleotide microarrays (Lockhart, D. J., et al., Nat Biotechnol 14 (1996) 1675-80), the, transcript levels in training set samples were measured for those genes that were represented on the microarray. The values for “transcription strength” were determined by averaging the values of a set of probes which were compared to a set of nearly identical probes containing a single mismatch. This was performed by using; methods provided by the oligonucleotide array of Affymetrix Inc.

### Example 2

### (E) Principle Components Analysis, Classifier and DecisionsTrees

In order to obtain comparable values between different samples, they had to be standardized first. The method followed that described (Lockhart, D. J., et al., Nat Biotechnol 14 (1996) 1675-80), except that correcting for (additive) background had been omitted. In brief, the data from one of the samples were declared to serve as a “standard”, and the values from all other samples were adapted to this standard. For every possible comparison to this standard, a set of “reliable” values was determined by calculating the correlation coefficient for a series of intervals of increasing length. The lower bound of reliability was the bound of the interval that had a correlation coefficient less than or equal to the smaller intervals. From all reliable values, 2 (logarithmized) correction factor was calculated by computing the median of the differences of the logarithmic values. Values that were zero or negative prior to taking the logarithm were not taken into account.

The obtained data matrix contained values from one sample per column. The gene expression profile across all samples for one gene or gene fragment represented on the oligonucleotide microarray was contained in a row of the matrix. To allow for rapid calculation of the classifier and to reduce memory usage, certain genes were pre-selected from the set of all genes represented on the array. The following criteria were applied:

$$\begin{matrix}Formula ( 1 ) : \\ {\sum }_{i = 1}^{k} \vert {μ}_{i} - \overline{μ} \vert - {\sum }_{i = 1}^{k} {σ}_{i} > 0 & \\ Formula ( 2 ) : \\ r < \frac{{\sum }_{i = 1}^{k} \vert {μ}_{i} - \overline{μ} \vert}{{\sum }_{i = 1}^{k} {σ}_{i}} & \end{matrix}$$

μ₁ refers to the average of the i-th class (i=1, . . . ,k), μ to the total average, σᵢ to the standard deviation of the i-th class and t to an arbitrary treshold ≦1. Selection by these methods resulted typically in a reduction in the number of genes by a factor of 10-30. To check the quality of the selection procedure, the first two principal components (Jolliffe, Principle Components Analysis (1986), Springer (New York)) for the samples were plotted. This allowed to judge whether or not a rigorous discrimination was possible between the different classes.

For construction of the classifier, decision trees (Breiman et al., Classification and regression try, Wadsworth & Brooks/Cole (Monterey)) were used. Simple decision trees that discriminate between n classes by using only transcription levels for (n−1) genes were used. They were trained and the selected genes were the discarded from the original data set. A new tree was constructed by using the truncated data set and the entire procedure was iterated until a predetermined number of trees was reached. The optimal number of trees could be estimated by counting the number of misclassifications of classifiers built from different numbers of trees. For this, an independent data set of cross-validation had to be used. The final vote of the multi-classifier was obtained by applying a vote-by-majority rule to the predictions of the contained trees. In the example of the present invention 15 decision trees had been used for the multi-classifier. This allowed perfect classification of 100% of the samples, discriminating between classes that were given by chromosomal aberrations. To estimate generalization properties, i.e. how accurate the classifier may perform on samples that have not been used for training, cross-validation had been used (Efron and Tibshirani, An introduction to the bootstrap (1993), Chapman & Hall (New York, London), pp. 237-247).

### Example 2

### Results (Golub Method)

From this point of view it was found that a set of 17 genes was sufficient to distinguish distinct AML subtypes from each other with high precision (Tables 1).

The classification model was able to identify the 4 morphologically and 3 cytogenetically and molecular biological different subtypes AML with t(8;21), with t(15;17), and with inv(16) (FIGS. 1a-b, 2).

In conclusion by comparison of gene expression profiles of AML samples (3 tested genetic subtypes t(8;21), t(15;17) and inv(I6)) genes could be identified which allowed a differentiation between each individual AML subtype in detail could be shown for the first time that these distinct abnormalities on the genomic level relate to a specific gene expression pattern. In other words, in the experimental setting the knowledge of the expression status of these designated genes was sufficient to predict the genetic abnormality and allows the diagnosis of specific genetically defined subtypes of AML (Table 1).

Results of methods described in I(E) are shown in Table 2 and FIGS. 3a+b, 1/2 and 4.

### Example 2

II) Pair-wise comparisons between normal bone marrow, AML ALL, CML, and CLL: By pair-wise comparisons gene expression profiles of 8 cases of normal bone marrow, 48 AML, 9 ALL, 8 CML, and 7 CLL were evaluated. These led to the identification of subtype-specific genes (Tables 3-12. FIGS. 5a-c, 6a-c, 7a-c, 8a-c).

### Example 2

### III) AML Classified According to WHO Proposal

To allow classification of AML subtypes according to the new WHO proposal we used the gene expression profiles of four genetically defined AML subtypes (t(8;21) n=9; t(15;17) n=18; inv(16) n=10; 11q23/MLL aberrations n=11). This led to the identification of subtype-specific genes (Table 13, FIGS. 9a-c).

### Example 2

IV) Normal bone marrow versus distinct genetic subtypes of AML: We used the gene expression profiles of normal bone marrow (n=8) and of four genetically defined AML subtypes (t(8;21) n=9; t(15;17) n=18; inv(16) n=10; 11q23/MLL aberrations n=10). This led to the identification of genes that allow the distinction between normal bone marrow and each of the four AML subtypes (Table 14).

### Example 2

V) Identification of genes specifically separating normal bone marrow, AML, ALL, CML, and CLL: We used the gene expression profiles of normal bone marrow (n=8) and of AML (n=48), ALL (n=9), CML (n=8), and CLL (n=7). This led to the identification of xx genes that allow the distinction between normal bone marrow and each of the four leukemia subtypes (Table 15, FIGS. 10a-c).

### Example 3

### Gene Expression Profiling Provides a Global and Robust Diagnostic Tool for Leukemia

### Example 3

### Introduction

The expression profiles of 12,600 genes were analyzed in 103 patients suffering from chronic myeloid leukemia (CML), chronic lymphoid leukemia (CLL), acute lymphoblastic leukemia (ALL), and acute myeloid leukemia (AML). A set of 71 genes shown in table 16 to 19 was identified as the minimal set necessary to accurately diagnose prognostically relevant leukemia subtypes and to distinguish these from normal bone marrow (BM, n=8). Thus, microarray technology is a suitable method for diagnosis of leukemia.

Today, the classification of hematological malignancies according to the WHO criteria describes chronic myeloid leukemia (CML), chronic lymphoid (CLL), acute lymphoblastic (ALL), and acute myeloid leukemia (AML). Within the latter two several prognostically relevant subtypes are established (see example 4). This subclassification is based on genetic abnormalities of the leukemic blasts associated with different prognoses and becomes increasingly important to guide therapy. Thus, the development of new, specific treatment approaches requires the precise identification of these subtypes that may benefit from individual therapeutic protocols. It has already been shown that the development of drugs targeting molecular aberrations can dramatically improve outcome. The introduction of all-trans retinoic acid (ATRA) into the treatment of AML with t(15;17)(q22;q11-12) has improved outcome from about 50% to 80% long-term survivors (1). In CML patients imatinib, a designed molecule that inhibits the t(9;22)(q34;q11) specific chimeric tyrosine kinase BCR-ABL, induces dramatically higher response rates as compared to conventional drugs (2). To fully take advantage of specific treatment options a precise identification of distinct leukemia subtypes is mandatory. However, standard diagnostics of leukemia using a combination of complementary methods is expensive, time-consuming, and requires experienced specialists.

Since its introduction, microarrays have been promising tools for basic research. With regard to leukemia, the pivotal discrimination of unselected ALL and AML samples based on their gene expression signatures inspired numerous studies (3). Recently, subtypes of childhood ALL could be correlated to specific gene expression profiles leading to both marker genes suitable for Initial diagnostics and canditates as predictors for outcome (Yeoh, Eng-Juh. pediadric ALL expression profiling Cancer Cell, 2002). Additionally, novel entities in hematological malignancies could be identified based on their distinct expression pattern as has been shown for multiple myeloma, large cell lymphoma, and childhood ALL (4-6). In example 4, it is demonstrated that cytogenetically defined AML subtypes can be correlated to specific gene expression profiles (see example 4). AML FAB M2 with t(8;21)(q22;q22), FAB M3/M3v with t(15;17)(q22;q11-12), or M4eo with inv(16)(p13q22) could be classified based on a minimal set of 13 genes. These genes belong to a large variety of different functional classes including members of signaling pathways, cell surface antigens, as well as plasma glycoproteins. Several genes are known to be involved in cytoskeletal structure, transcriptional processes, or have not yet further been functionally described.

Here, gene expression profiles of 103 leukemia patients were acquired representing 11 groups and eight normal BM donors to designate leukemia-specific genes which build up the basis for a novel diagnostic tool. Following the aims of Golub, who introduced the cancer class prediction methodology (3, 7), all four major leukemia types were analyzed and also included cytogenetically defined subgroups of AML and ALL as described in the WHO classification of leukemia, respectively (FIG. 11a). All patient samples were thoroughly characterized combining cytomorphology, cytogenetics, immunophenotyping, and molecular genetics. This was a prerequisite to obtain disease-specific gene expression profiles for each entity. We used Affymetrix expression probe arrays HG-U95Av2 to interrogate the mRNA abundance of approximately 12,600 transcripts. In order to identify genes suitable for a leukemia prediction classifier we applied a slightly modified prediction methodology as introduced by Golub [see (Note1_Golub method)]. A minimal set of candidate genes had to show both maximal classification accuracy and maximal confidence. Accuracy of the classifiers was determined by permutation-based neighborhood analysis [see (Note2_leave-one-out crossvalidation)]. Additional information about the absolute differences of expression intensities and further descriptions of all candidate genes can be found in the supporting online material.

In a first step, based on 23 informative genes the samples were assigned to either normal BM, CLL, CML, ALL, or AML, respectively (Table 22; Description of Table 22: Classification scheme for 4 major leukemia types and normal BM. Matrices delineate distribution of actual leukemia types as compared with predicted types from pairwise comparisons. Class assignment can be based on the expression profiles of 23 genes. Except for pairwise comparison of AML versus ALL, all cases can be predicted accurately in leave-one-out cross validation with 100% accuracy and strong confidence. For each pairwise comparison the minimal set of informative genes is represented by approved HUGO Gene Nomenclature Committee (HGNC) symbols. Not yet approved genes are marked by asterisks.). In 9/10 pairwise comparisons all samples were classified correctly (335 individual assignments; 100% accuracy). In one comparison (AML versus ALL) 75/77 samples were classified correctly resulting in an accuracy of 97%. Two ALL samples were misclassified as AML. This may be due to the heterogenity of both groups (n=18 versus n=59) causing noise in the expression data.

For each pairwise comparison a set of discriminative genes is disclosed in table 20 whereby the gene names can be found in table 21. The most discriminative and informative genes are marked by asterisks in table 20 and are the 71 genes shown in table 16 to 19

In detail, we found phospholipidscramblase 1 (PLSCR1) to be lower expressed in AML and ALL as compared to normal BM. PLSCR1 encodes for a plasma membrane protein and has been proposed to play a role in transbilayer migration of phospholipids and in recognition and phagocytic clearance of injured, aged, or apoptotic cells (8). The biologic effects of interferon-alpha may be mediated by PLSCR1 which is markedly upregulated by interferon (9, 10). We also observed that LEF-1 was absent in myeloid leukemias but highly expressed in lymphoid leukemias. LEF-1 was shown to be mitogenic and important for cell survival in pro-B cells (11). The B-cell specific coactivator of octamer binding transcription factors, POU2AF1, plays an important role in the antigen-driven stages of B cell activation and maturation and discriminates between AML and CLL (12). MSF has been reported to be a translocation partner of the mixed-lineage leukemia gene (MLL) in AML and was able to separate AML from ALL (13). Likewise, OS-9, not yet further functionally described except for amplification in osteosarcomas, was differentially expressed between AML and ALL (14). HLA-DMB plays a critical role in antigen presentation by catalyzing the release of class II HLA-associated invariant chain binding sites for acquisition of antigenic peptides (15). It is known that lymphocytes in CLL express high levels of class II antigens whereas mature myeloid leukemias are e.g. HLA-DR negative (16, 17). Therefore, the differential expression of HLA-DMB in CML as compared to CLL illustrates well the differential expression of cell surface MHC class II molecules. NCOA1 plays a critical role in STAT3 and STAT6 pathways and was expressed higher in CLL as compared to ALL suggesting an inhibitory effect of STAT6-mediated transactivation in CLL (18). A member of the tumor necrosis factor receptor family, whose surface expression has already been reported in CLL (19), CD27, was identified to assign samples either ALL or CLL. We also detected LCN2 that was shown to be a modulator of inflammation regulated by interleukin-9 with highest expression in CML samples (20). IRF4, an immune system-restricted interferon regulatory factor that is required for lymphocyte activation showed no expression in CML while it was expressed in normal BM. Recently, an increase of IRF4 levels in CML patients demonstrated an association with a good response to interferon-alpha therapy (21). Several other proteins (DEFA3, SGP28, CAMP, CLC) are known to be stored in the granules of neutrophils and allowed assignment of leukemic samples to the CML type if highly expressed (22-25).

The second step of our approach was to build up a classifier for the identification of AML subtypes genetically defined according to the WHO classification, i.e. AML with t(8;21), with t(15;17) with inv(16), and with 11q23-translocations involving the MLL gene, respectively. In addition, a category ‘other’ was analyzed comprising AML with normal karyotype (n=3), AML with complex aberrant karyotype (n=4), and AML with trisomy 8 as sole abnormality (n=3), respectively. A set of 25 most informative genes was identified based on pairwise comparisons and one-versus-all (OVA) comparisons. None of these genes had already been identified for the classification of the four leukemia types and normal BM as described above. As shown in FIG. 11b, distinct AML subgroups cluster together due to homogeneous expression profiles. This classification model showed 100% classification accuracies in 14/15 comparisons (440 individual assignments). In one OVA comparison, ‘other’ versus all other AML, 54/55 samples were assigned correctly. The misclassification of one sample may also reflect the large heterogenity of both groups.

The following genes were identified in OVA comparisons and discriminate distinct AML subtypes. The gene most valuable for prediction of AML M4eo with inv(16) was MYH11. Its higher expression as compared to all other AML most probably is due to hybridization of the M4eo-specific fusion transcripts CBFβ-MYH11 to corresponding MYH11-oligonucleotides represented on the microarray (26). Likewise, the higher expression of CBFA2T1 (formerly ETO) in AML with t(8;21) may be due to a similar effect of hybridization of the subtype-specific AML1-ETO fusion transcript (27). Another highly characteristic gene for t(8;21) positive AML was POU4F1, which has been described to play an important role in retinal ganglion cell differentiation and has recently been shown to confer an oncogenic potential when co-transfected with H-RAS (28). Furthermore, it was shown to be highly expressed in neuro-epithelioma and ewing sarcomas (29). Another member of this transcription factor family, POU2F2, was able to discriminate between t(11q23)/MLL versus group ‘other’. A related gene, POU2AF1, has recently been reported to be underexpressed in acute leukemia with t(11q23)/MLL-rearrangement (5). The most informative genes in our approach discriminating AML with t(11q23)/MLL-rearrangement from all other AML subtypes were SOCS-2 and MBNL. Generally, SOCS-2 shows a higher expression level in AML with t(11q23)/MLL-rearrangement and is known to play a role in cytokine-induced signaling pathways (30). Similarly, MBNL shows a higher expression in AML with t(11q23)/MLL-rearrangement as compared to all other AML samples. Its encoded protein as well as other MBL family members are localized in the nucleus and share a Cys3His zinc finger motif (31). MBL proteins occur in several isoforms due to alternative splicing (32) and may have different functions as has been shown for HOX genes (33). HOXA9 has been reported to be highly expressed in leukemia with MLL-rearrangements (5). In contrast, expression of HOXB5 is characteristic of AML group ‘other’ as compared to all other AML subtypes in our data. The most important genes discriminating AML with t(15;17) from all other AML subtypes were ARGHGAP4 and CTSW. ARGHGAP4 is predominantly expressed in hematopoietic cells but showed a lower expression level in AML with t(15;17) as compared to all other AML subtypes. It encodes a member of signaling proteins involved in regulation of small GTP-binding proteins of the RAS-superfamily, which themselves play an important role in cell cycle and apoptosis (34). CTSW encodes for a recently described papain-like cysteine protease, which is predominantly expressed in NK cells and to a lesser extent in cytotoxic lymphocytes. It may represent a putative component of the endoplasmatic reticulum resident proteolytic machinery (35). A survey about the expression levels of genes in the AML subtypes can be found in FIG. 12a-d

Subclassification of ALL comprising the three B-lineage groups ALL with t(9;22), with t(4;11), or with t(8;14) was analyzed next and compared with T-lineage ALL expression profiles. All samples were classified correctly on the basis of 19 genes (FIG. 11c). This set included TRB, which was already described to distinguish between CLL and CML (Table 22).

In detail, the genes encoding for the T cell receptor beta subunit and T cell surface CD3 delta chain (TRB, CD3D) were identified as highly indicative of T-ALL as compared to both ALL with t(9;22) and all other ALL subtypes. This is in line with standard diagnostics of T-ALL by immunophenotyping where these antigens comprise the most specific ones (36). Similarly, MME (formerly CD10) was highly expressed in ALL with t(9;22) only. This on the one hand may reflect that t(9;22) is observed in common-ALL and in pre-B ALL only. On the other hand, this data again demonstrates that the gene used for diagnostic purposes in flow cytometry, MME, may be highly indicative of these ALL subtypes in comparisons to the more immature B-lineage ALL, i.e. pro-B ALL, as well as the mature B-ALL and the T-ALL. Furthermore, the identification of connective tissue growth factor (CTGF) as a specific marker for ALL with t(4;11) adds to previous data demonstrating its increased gene expression in childhood ALL in general (37). The glucocorticoid receptor beta has been shown to be highly expressed in ALL with t(4;11) but not in t(9;22) positive ALL. This is in line with the particularly poor prognosis of the latter subtype since response to corticoid therapy is one of the most powerful prognostic factors in ALL (38, 39). In addition, we speculate that new treatment options may be realized for T-ALL based on the high expression of adenosine deaminase (ADA) in this subtype. Inhibitors of ADA have been shown to be effective in indolent T-cell lymphomas but have not yet been evaluated in T-ALL (40). One cytokine differentially expressed between t(8;14) positive ALL and T-lineage ALL was SCYA3. We recommend testing the monitoring of its protein expression as a supplemental antigen useful for immunophenotypical identification of t(8;14) positive ALL. Finally, in ALL carrying t(4;11) v-myb is highly expressed and may thus be involved in the pathogenesis of this subtype. In general, a role of v-myb has been described for the transformation of myelomonocytic cells (41). A survey about the expression levels of genes in the AML subtypes can be found in FIGS. 12e-12i.

At least, we intended to separate t(9;22) positive from t(9;22) negative ALL. Our data contained two genes encoding for ADCY3 and the hypothetical protein KIAA1013 which were sufficient for the 100% correct assignments of 18 analyzed cases. Both genes showed a higher expression in t(9;22) positive as compared to t(9;22) negative ALL. Additionally, distinguishing B lineage from T-lineage ALL, CD3D and TRB repeatedly showed their usefulness as T-ALL marker genes as already described in FIG. 11c (18/18 correct individual assignments).

Generally, chromosomal aberrations are strongly associated with morphological characteristics. However, there are two chromosomal aberrations which are observed in both myeloid and lymphatic neoplasms, i.e. t(11q23)/MLL and the t(9;22). The t(9;22) occurs in ALL and CML, and t(1 q23)/MLL is observed in ALL and AML, respectively. Analyzing gene expression signatures of both t(9;22) positive ALL and CML we identified two genes, which allowed 17/17 correct lineage assignments. CD74 plays a critical role in MHC class II antigen processing and demonstrated a lower expression in t(9;22) positive CML (42). This may also explain the relationship between the low MHC class II antigen presentation in CML in general and fits well to the recognized lower HLA-DMB expression in CML as compared to CLL (Table 1). CAPN3 is a member of the papain superfamily and was higher expressed in CML discriminating them from t(9;22) positive ALL [see (Note_{—}38894_g_at)].

In addition, our results indicate that the expression signatures of two genes, CD24 and CTGF, are sufficient for 14/14 correct assignments of the t(11q23)/MLL positive leukemias either to ALL or to AML. Thus, in both scenarios lineage assignment can be accomplished based on specific gene expression signatures despite the same underlying chromosomal aberrations.

Taken together, these data demonstrate the utility of gene expression profiling using microarrays for diagnosis of leukemia. In total, 11 different leukemia entities could clearly be distinguished from each other and from normal BM, respectively. These leukemias are associated with highly differing prognoses and require specific treatment strategies. By performing these analyses on a single platform requiring basic molecular biological methods, this approach guarantees broad access to high-quality diagnosis of leukemia. The robust gene expression analysis with high diagnostic accuracy can substitute the combination of cytomorphology, cytogenetics, immunophenotyping, and molecular biological methods used today. Compared to the combination of methods used so far, this approach also reduces costs. In order to introduce diagnostical genomics into routine clinical practice, prospective trials in parallel to conventional methods are necessary to prove the reliability in a large cohort of patients. Furthermore, gene expression patterns will allow the additional subclassification of leukemia especially in subtypes with no specific cytogenetic markers and the identification of deregulated master genes within distinct leukemia entities can guide the way to new therapeutic approaches.

### REFERENCE LIST OF EXAMPLE 3

1. Fenaux, P., Le Deley, M. C., Castaigne, S., Archimbaud, E., Chomienne, C., Link, H., Guerci, A., Duarte, M., Daniel, M. T., Bowen, D. et al. (1993) Blood 82, 3241-3249.
2. Kantarjian, H., Sawyers, C., Hochhaus, A., Gulhot, F., Schiffer, C., Gambacorti-Passerini, C., Niederwieser, D., Resta, D., Capdeville, R., Zoellner, U. et al. (2002) N. Engl. J. Med. 346, 645-652.
3. Golub, T. R., Slonim, D. K., Tamayo, P., Huard, C., Gaasenbeek, M., Mesirov, J. P., Coller, H., Loh, M. L., Downing, J. R., Caligiuri, M. A. et al. (1999) Science 286, 531-537.
4. Alizadeh, A. A., Eisen, M. B., Davis, R. E., Ma, C., Lossos, I. S., Rosenwald, A., Boldrick, J. C., Sabet, H., Tran, T., Yu, X. et al. (2000) Nature 403, 503-511.
5. Armstrong, S. A., Staunton, J. E., Silverman, L. B., Pieters, R., den Boer, M. L., Minden, M. D., Sallan, S. E., Lander, E. S., Golub, T. R. & Korsmeyer, S. J. (2002) Nat. Genet. 30, 41-47.
6. Zhan, F., Hardin, J., Kordsmeier, B., Bumm, K., Zheng, M., Tian, E., Sanderson, R., Yang, Y., Wilson, C., Zangari, M. et al. (2002) Blood 99, 1745-1757.
7. Pomeroy, S. L., Tamayo, P., Gaasenbeek, M., Sturla, L. M., Angelo, M., McLaughlin, M. E., Kim, J. Y., Goumnerova, L. C., Black, P. M., Lau, C. et al. (2002) Nature 415, 436-442.
8. Fadok, V. A., Bratton, D. L., Rose, D. M., Pearson, A., Ezekewitz, R. A. & Henson, P. M. (2000) Nature 405, 85-90.
9. Der, S. D., Zhou, A., Williams, B. R. & Silverman, R. H. (1998) Proc. Natl. Acad. Sci. U.S. A 95, 15623-15628.
10. Zhou, Q., Zhao, J., Al Zoghaibi, F., Zhou, A., Wiedmer, T., Silverman, R. H. & Sims, P. J. (2000) Blood 95, 2593-2599.
11. Reya, T., O'Riordan, M., Okamura, R., Devaney, E., Willert, K., Nusse, R. & Grosschedl, R. (2000) Immunity. 13, 15-24.
12. Nielsen, P. J., Georgiev, O., Lorenz, B. & Schaffner, W. (1996) Eur. J. Immunol. 26, 3214-3218.
13. Osaka, M., Rowley, J. D. & Zeleznik, L. (1999) Proc. Natl. Acad. Sci. U.S. A 96, 6428-6433.
14. Su, Y. A., Hutter, C. M., Trent, J. M. & Meltzer, P. S. (1996) Mol. Carcinog. 15, 270-275.
15. Van Kaer, L. (2001) Immunol. Res. 23, 205-214.
16. Marti, G. E., Zenger, V., Caproaso, N. E., Brown, M., Washington, G. C., Carter, P., Schechter, G. & Noguchi, P. (1989) Anal. Quant. Cytol. Histol. 11, 315-323.
17. Newman, R. A. & Greaves, M. F. (1982) Clin. Exp. Immunol. 50, 41-50.
18. Litterst, C. M. & Pfitzner, E. (2001) J. Biol. Chem. 276, 45713-45721.
19. van Oers, M. H., Pals, S. T., Evers, L. M., van der Schoot, C. E., Koopman, G., Bonfrer, J. M., Hintzen, R. Q., dem Borne, A. E. & van Lier, R. A. (1993) Blood 82, 3430-3436.
20. Cowland, J. B. & Borregaard, N. (997) Genomics 45, 17-23.
21. Schmidt, M., Hochhaus, A, Konig-Merediz, S. A., Brendel, C., Proba, J., Hoppe, G. J., Wittig, B., Ehninger, G., Hehimann, R. & Neubauer, A. (2000) J. Clin. Oncol. 18, 3331-3338.
22. Oren, A. & Taylor, J. M. (1995) J. Lab Clin. Med. 125, 340-347.
23. Cowland, J. B. & Borregaard, N. (1999) J. Leukoc. Biol. 66, 989-995.
24. Dvorak, A. M., Letourneau, L., Login, G. R., Weller, P. F. & Ackerman, S. J. (1988) Blood 72, 150-158.
25. Gudmundsson, G. H., Agerberth, B., Odeberg, J., Bergman, T., Olsson, B. & Salcedo, R. (1996) Eur. J. Biochem. 238, 325-332.
26. van der Reijden, B. A., Dauwerse, J. G., Wessels, J. W., Beverstock, G. C., Hagemeijer, A., van Ommen, G. J. & Breuning, M. H. (1993) Blood 82, 2948-2952.
27. Downing, J. R., Head, D. R., Curcio-Brint, A. M., Hulshof, M. G., Motroni, T. A., Raimondi, S. C., Carroll, A. J., Drabkin, H. A., Willman, C., Theil, K. S. et al. (1993) Blood 81, 2860-2865. Liu, W., Khare, S. L., Liang, X., X., Peters M. A., Liu, X., Cepko, C. L. & Xiang M. (2000) Development 127, 3237-3247.
29. Leblond-Francillard, M., Picon, A., Bertagna, X. & de Keyzer, Y. (1997) J. Clin. Endocrinol. Metab 82, 89-94.
30. Krebs, D. L. & Hilton, D. J. (2001) Stem Cells 19, 378-387.
31. Begemann, G., Paricio, N., Artero, R., Kiss, I., Perez-Alonso, M. & Mlodzik, M. (1997) Development 124, 4321-4331.
32. Fardaei, M., Rogers, M. T., Thorpe, H. M., Larkin, K., Hamshere, M. G., Harper, P. S. & Brook, J. D. (2002) Hum. Mol. Genet. 11, 805-814.
33. van Oostveen, J., Bijl, J., Raaphorst, F., Walboomers, J. & Meijer, C. (1999) Leukemia 13, 1675-1690.
34. Tribioli, C., Droetto, S., Bione, S., Cesareni, G., Torrisi, M. R., Lotti, L. V., Lanfrancone, L., Toniolo, D. & Pelicci, P. (1996) Proc. Natl. Acad. Sci. U.S. A 93, 695-699.
35. Wex, T., Buhling, F., Wex, H., Gunther, D., Malfertheiner, P., Weber, E. & Bromme, D. (2001) J. Immunol. 167, 2172-2178.
36. Campana, D. & Behm, F. G. (2000) J. Immunol. Methods 243, 59-75.
37. Vorwerk, P., Wex, H., Hohmann, B., Mohnike, K., Schmidt, U. & Mittler, U. (2002) Mol. Pathol. 55, 40-45.
38. Gleissner, B., Gokbuget, N., Bartram, C. R., Janssen, B., Rieder, H., Janssen, J. W., Fonatsch, C., Heyll, A., Voliotis, D., Beck, J. et al. (2002) Blood 99, 1536-1543.
39. Panzer-Grumayer, E. R., Schneider, M., Panzer, S., Fasching, K. & Gadner, H. (2000) Blood 95, 790-794.
40. O'Brien, S., Kurzrock, R., Duvic, M., Kantarjian, H., Stass, S., Robertson, L. E., Estey, E., Pierce, S. & Keating, M. J. (1994) Blood 84, 733-738.
41. Dvorakova, M., Kralova, J., Karafiat, V., Bartunek, P. & Dvorak, M. (2001) Blood Cells Mol. Dis. 27, 437-445.
42. Villadangos, J. A. (2001) Mol. Immunol. 38, 329-346.
   Notes of Example 3
   [see (Note1_Golub method)]

When comparing two groups of microarray experiments, Golub's method sorts the genes with respect to the signal-to-noise ratio of gene x: Sₓ=(μ₁−μ₂)/(σ₁+σ₂), where μk and σk denote the mean expression and standard deviation of gene x in group k. According to a specified number of “informative” genes (e.g. 20) the best discriminating genes are selected. For each informative gene a decision limit is calculated as bₓ=(μ₁+μ₂)/2. To classify a new sample of an independent test set, the gene expression levels of informative genes are taken and for each gene x and sample y a so-called vote is calculated as Vₓ=Sₓ(gₓ^{y}−bₓ), where gₓ^{y} denotes expression level of gene x in sample y. The votes of all informative genes are summed up (“weighted voting”) and depending upon the sign of this sum the new sample is classified as group 1 or group 2. The confidence in the prediction is calculated as |ΣVₓ/Σ|Vₓ||.

However, the decision limit proposed by Golub does not provide optimal classification accuracy in all situations. Importantly, when the standard deviation of expression levels within the two groups are very different, the decision limit is biased towards the group with the higher standard deviation. A decision limit for a particular gene can be considered optimal, if it achieves maximum classification accuracy for a given dataset. By determining systematically classification accuracies for a set of possible decision limits, an optimal decision limit can be calculated. We selected an optimal decision limit from the following set of decision limits Lₓ: Lₓ={(gₓ^{y}+gₓ^{y-1})/2|1<y<=n} where gₓ^{y} denotes expression level of gene x in sample y, n denotes the total number of samples in the training set.

Additionally, we applied an heuristic approach to select a minimal set of discriminative genes, which provides maximum classification accuracy in leave-one-out-crossvalidation. We applied for a given set of 20 informative genes weighted voting as described above and the classification accuracy was calculated by crossvalidation. Therefore, our algorithm consists of the following steps: (i) Calculate the top 20 discriminating genes according to the signal-to-noise ratio. (ii) Calculate classification accuracy and confidence based on optimal decision limits for each of the top 20 genes (iii) Select the gene which provides best classification accuracy and confidence out of step 2. (iv) Test for each of the remaining 19 genes, whether adding this gene to the model improves accuracy and confidence; if the gene improves accuracy and confidence, it is added to the weighted voting model, otherwise it is discarded.

In detail, this method can be described as follows:

### Example 3

### Subheading to Note1_Golub Method: Abstracts

Differentially expressed genes can potentially be used in medical diagnostics, if the gene expression patterns are reliable and specific for a particular disease. diffgenes is a program to identify differentially expressed genes in microarray experiments. Its algorithm is based on the method proposed by Golub, but contains two improvements: an optimized decision limit per gene and a minimal set of discriminative genes.

The new method was applied to a human dataset from the domain of cancer research consisting of 103 microarrays with 12625 genes each. diffgenes outperforms Golub's method clearly both in terms of accuracy and confidence of classifications. The biological validation of the results is facilitated, because diffgenes identifies a very small number of candidate genes (typically <5). Microarray datasets can be analyzed with diffgenes on the Internet at http://martin-dugas.de/diffgenes/

### Example 3

### Subheading to Note1_Golub Method: Introduction

Microarrays are used in ongoing research to characterize disease processes on a molecular level. Gene expression analysis enables to identify new subtypes within known diseases with prognostic relevance for the patients [Alizadeh 2000].

For interpretation of the wealth of data—more than 10.000 parameters per experiment—it is advisable to integrate microarray data with detailed clinical information. For applications in medical diagnostics, significant associations between gene expression profiles and sample groups resulting in classification accuracies in the range of 70-80% are not sufficient; for diagnostic purposes at least 95% classification accuracy is required.

If a certain disease is characterized by a specific gene product, e.g. a pathologic fusion gene, a precise measurement of the expression of this particular gene should be a reliable marker for the disease. Therefore in a diagnostic setting, very few and specific genes would be desirable.

However, for many diseases the precise molecular pathogenesis is not yet known. In addition, the function of many genes on currently available microarrays like Affymetrix GeneChip® is still unclear.

Therefore microarray data should be analyzed and interpreted carefully. By integration of data from different diagnostic modalities (morphology, PCR, FISH, clinical data) the biological plausibility and consistency of microarray data can be verified.

### Example 3

### Subheading to Note1_Golub Method: Methods

### Example 3

### Subheading to Note1_Golub Method: Golub's Method

When comparing two groups of microarray experiments, Golub's method sorts the genes with respect to the signal-to-noise ratio of gene x: Sₓ=(μ₁−μ₂)/(σ₁+σ₂), where μₖ and σₖ denote the mean expression and standard deviation of gene x in group k.

According to a specified number of “informative” genes (e.g. 20) the best discriminating genes are selected. For each informative gene a decision limit is calculated as bₓ=(μ₁+/μ₂)/2. To classify a new sample of an independent test set, the gene expression levels of informative genes are taken and for each gene x and sample y a so-called vote is calculated as Vₓ=Sₓ(gₓ^{y}−bₓ), where gₓ^{y} denotes expression level of gene x in sample y. The votes of all informative genes are summed up (“weighted voting”) and depending upon the sign of this sum the new sample is classified as group 1 or group 2. The confidence in the prediction is calculated as |Σ Vₓ/Σ|Vₓ||. To assess the significance of each gene, a permutation test is performed, which determines signal-to-noise ratios when class labels are permuted randomly. To assess the robustness of the classifier, a leave-one-out crossvalidation is performed. Accuracy is the rate of correctly classified test samples. Further details are contained in [Golub 1999], [Pomeroy 2002, Supplement].

### Example 3

### Subheading to Note1_Golub Method: An Optimized Decision Limit

The decision limit proposed by Golub does not provide optimal classification accuracy in all situations. As can be seen in FIG. 13a, when the standard deviation of expression levels within the two groups are very different, the decision limit is biased towards the group with the higher standard deviation.

A decision limit for a particular gene can be considered optimal, if it achieves maximum classification accuracy for a given dataset. By determining systematically classification accuracies for a set of possible decision limits, an optimal decision limit can be calculated. The diffgenes program selects an optimal decision limit from the following set of decision limits Lₓ:
Lₓ{(gₓ^{y}+gₓ^{y-1})/2|1<y<=n}
where gₓ^{y} denotes expression level of gene x in sample y, n denotes the total number of samples in the training set.

### Example 3

### Subheading to Note1_Golub Method: A Minimal Set of Discriminative Genes

Golubs method selects an arbitrary number of “informative” genes to discriminate between two classes of samples according to their signal-to-noise ratio, typically in the range of 10 to 50 genes. Choosing too many genes carries the risk of overfitting, which causes poor generalization features of the model. Therefore diffgenes applies an heuristic approach to select a minimal set of discriminative genes, which provides maximum classification accuracy in leave-one-out-crossvalidation. I.e. for a given set of genes weighted voting as described by Golub is applied and the classification accuracy is calculated by crossvalidation.

The diffgenes algorithm consists of the following steps:
1. Calculate the top 20 discriminating genes according to the signal-to-noise ratio
2. Calculate classification accuracy and confidence based on optimal decision limits for each of the top 20 genes
3. Select the gene which provides best classification accuracy and confidence out of step 2.
4. Test for each of the remaining 19 genes, whether adding this gene to the model improves accuracy and confidence; if the gene improves accuracy and confidence, it is added to the weighted voting model, otherwise it is discarded.

### Example 3

### Subheading to Note1_Golub Method: Results

The method was applied to a new human dataset from the domain of cancer research consisting of 103 Affymetrix Genechip(R) microarrays with 12625 genes each. Table 23 presents an analysis of 18 samples class A versus 85 samples class non-A (Description of Table 23: Analysis of 18 samples class A versus 85 samples class non-A. On the left the analysis according to Golub is presented for 20 informative genes. The crossvalidation accuracy is 0,87, confidence 0,77. Samples, where crossvalidation failed, are listed. For each gene signal to noise ratio, p-value (significance obtained from permutation test) and decision limit are provided. On the right the same data set is analyzed using diffgenes. By selection of 3 genes (marked with asterisks) out of the top 20 genes and selecting optimized decision limits, the crossvalidation accuracy reaches 0,96, confidence 0,88.). Based on 20 informative genes Golub's method results in a crossvalidation accuracy of 0,87 (confidence 0,77); diffgenes achieves with three genes out of the top 20 set a crossvalidation accuracy of 0,96 (confidence 0,88). The same analysis was performed for one versus all (OVA) and all pairs (AP) comparisons in this dataset consisting of 5 different classes. FIG. 13b presents accuracy and confidence obtained by both methods: diffgenes outperforms Golub's method clearly both in terms of accuracy and confidence of classifications. The same comparative approach was applied to two datasets in cardiology and cell biology consisting of 44 and 67 microarrays. The results concerning Golub's method and diffgenes were very similar (data not shown).

### Example 3

### Subheading to Note1_Golub Method: Discussion

There are two major challenges in the analysis of microarray data: the number of variables (genes) is much higher than the number of individual samples and the correlation structure of the parameters is widely unknown.

Golub's method to analyse microarray data has been applied to important medical datasets [Armstrong 2002]. Recently many different approaches have been applied to microarray data: Classical statistical techniques like ANOVA with adjustment for multiple testing, significance analysis of microarrays (SAM) [Tusher 2001], selection of discriminative genes with support vector machines (SVM), neural networks and many more. This indicates that the underlying problem is important and non-trivial; a comparison of different methods is needed. Robustness of the generated mathematical models is an important issue, therefore bootstrap procedures and permutation tests are applied.

For medical diagnostics differentially expressed genes are of interest, but the sensitivity and specificity for particular diseases must be validated prospectively in larger patient cohorts diffgenes is an extension of Golub's method to improve classification accuracy, which is very relevant in a diagnostic setting. The optimized decision limit plays an important role, because the situation presented in FIG. 13a is quite common in biological contexts: group 1 represents samples, where the expression of gene x is repressed while gene x is activated in group 2. The biological validation of the results is facilitated, because diffgenes identifies a very small number of candidate genes (typically <5).

Emphasis must be placed on verification of results by other diagnostic procedures, because the selected “important” genes are not only dependent on the statistics procedure, but also on the preprocessing of data. In our setting by integration of microarray analysis with other laboratory modalities (morphology, cytogenetics, molecular genetics, immunphenotyping) and clinical data the plausibility and consistency of results could be evaluated, therefore we are optimistic, that the demanding requirements for medical diagnostics can be fulfilled with microarray technology in the near future.

### Example 3

### Subheading to Note1_Golub Method: References

Alizadeh A A, Eisen M B, Davis R E, et al. (2000) Distinct types of diffuse large B-cell lymphoma identified by gene expression profiling. Nature 403(6769):503
Armstrong S A, Staunton J E, Silverman L B, et al. (2002) MLL translocations specify a distinct gene expression profile that distinguishes a unique leukemia: Nature Genetics 1:41-7
Golub T R, Slonim D K, Tamayo P, et al. (1999) Molecular classification of cancer: class discovery and class prediction by gene expression monitoring. Science 286(5439):531-7
Pomeroy S L, Tamayo P, Gaasenbeek M, et al. (2002) Prediction of central nervous system embryonal tumour outcome based on gene expression. Nature 415(6870):436-42
Tusher V G, Tibshirani R, Chu G (2001) Significance analysis of microarrays applied to the ionizing radiation response. PNAS 98: 5116-5121

### Example 3

### See (Note2_Leave-One-Out Crossvalidation)

To assess the significance of each gene, a permutation test is performed, which determines signal-to-noise ratios when class labels are permuted randomly. To assess the robustness of the classifier, a leave-one-out crossvalidation is performed. Accuracy is the rate of correctly classified test samples.

### Example 3

### See (Note_{—}38894_g_at)

The second top-ranked gene was represented by the Affymetrix probe set identifier: 38894_g_a. However, no clear gene assignment was possible for this informative prove set. Therefore, CAPN3 was chosen.

### Example 4

### PNAS

### Example 4

### Abstract

Acute myeloid leukemia (AML) is a heterogeneous group of genetically defined diseases. Their classification is important with regard to prognosis and treatment. We performed microarray analyses for gene expression profiling on bone marrow samples of 37 patients with newly diagnosed AML. All cases had either of the distinct subtypes AML M2 with t(8;21), AML M3 or M3v with t(15;17), or AML M4eo with inv(16). Diagnosis was established by cytomorphology, cytogenetics, fluorescence-in-situ hybridization, and RT-PCR in every sample. By using two different strategies for microarray data analyses, this study for the first time revealed a unique correlation between AML-specific cytogenetic, aberrations and gene expression profiles.

### Example 4

### Introduction

Acute myeloid leukemia (AML) is a heterogeneous group of diseases with respect to biology and clinical course. Since the introduction of the FAB-classification in 1976 diagnosis and classification have been based on cytomorphology and cytochemistry (1). As other techniques like immunophenotyping, cytogenetics, and molecular genetics contributed to the definition of AML subtypes the FAB-classification was updated. In 1999 the WHO classification for tumors of hematopoietic and lymphoid tissues was proposed. In an attempt to define biologically homogeneous entities which have clinical relevance morphologic, immunophenotypic, genetic and clinical features were incorporated (2, 3).

For optimal treatment approaches both a precise diagnosis and prognostic parameters that determine response to therapy and survival are needed. So far, the karyotype of the AML blasts is the most important independent prognostic factor. A favorable outcome under currently used treatment regimens with cure rates from 50% up to 85% was observed in several studies in patients with a) t(8;21)(q22;q22) occurring mostly in FAB subtype AML M2, b) inv(16)(p13q22) associated with AML M4eo and c) t(15;17)(q22;q11-12) associated with AML M3 and AML M3v(4-6). In contrast, chromosome aberrations with an unfavorable clinical course are −5/del(5q), −7/del(7q), inv(3)/t(3;3) and complex aberrant karyotypes with cure rates of less than 10% (7, 8). The remainder AML patients are assigned to a prognostically intermediate group. This latter group is very heterogeneous because it includes patients with a normal karyotype as well as those with rare chromosome aberrations and yet unknown prognostic impact.

Besides their prognostic impact genetic aberrations are involved in the pathogenesis of leukemia. While for unbalanced cytogenetic aberrations the heterogeneous pathogenetic mechanisms have not yet conclusively been determined, several studies provide strong evidence for the central pathogenetic role of leukemia-specific fusion genes that are generated by the above mentioned balanced abnormalities (9-12). Therefore it can be postulated that AML with balanced abnormalities most probably display a homogeneous gene expression profile and thus are promising candidates for microarray analyses.

In a pivotal study, gene expression profiles were analyzed in bone marrow samples of 27 ALL and 11 AML. A set of 50 genes out of 6,817 analyzed genes was sufficient to discriminate ALL and AML. By leave-one-out cross-validation it was possible to correctly classify 36 out of 38 acute leukemia cases. A class predictor could automatically determine new leukemia cases out of an independent test set as belonging to the myeloid or the lymphoid lineage. Thus, these results demonstrated the possibility of cancer classification based on gene expression profiling (13). In a further approach comparing AML with trisomy 8 and AML with normal karyotype expression profiling revealed fundamental biological differences in AML with isolated trisomy 8 and normal cytogenetics (14). More recently, acute lymphoblastic leukemias (ALL) with translocations involving the MLL gene could be separated from ALL cases without MLL translocations and from cases with AML by gene expression profiling (15).

The aim of our investigation was to answer the question whether a leukemia specific genotype is associated with a distinct gene expression profile. Therefore, we analyzed three distinct genetic subtypes of acute myeloid leukemia: t(8;21)(q22;q22), inv(16)(p13q22) and t(15;17)(q22;q12) which lead to subtype specific fusion genes AML1-ETO, CBFB-MYH11 and PML-RARA, respectively. They are specifically associated with four distinct morphological subtypes according to the FAB classification: AML M2, AML M4eo, AML M3 and AML M3v(16-18). We performed microarray analyses on a cohort of leukemia samples (n=37) and applied several methodologies to evaluate genes which allowed an assignment to the corresponding type of cytogenetic aberration for classification.

This is the first time that AML-specific cytogenetic aberrations can be correlated with corresponding gene expression profiles and vice versa.

### Example 4

### Methods

### Example 4

### Selection and Characterization of Leukemia Samples

For this investigation we selected bone marrow (BM) samples from 37 AML patients representing four morphological and three underlying cytogenetic subgroups. All cases were sent for reference diagnostics to our laboratory and registered in our leukemia database (19). Samples were received either locally or by overnight mail. All samples were newly diagnosed de novo AML and were characterized by cytomorphology, cytogenetics, FISH, and molecular genetics in each case. Gene expression analyses were performed on cells remaining from the diagnostic samples. Samples had been lysed immediately, frozen and were stored at −80° C. from one to 34 months until preparation for gene expression analysis.

### Example 4

### Cytomorphology

Analysis was based on May-Grünwald-Giemsa stain, myeloperoxidase reaction, and non-specific esterase reaction using alpha-naphthyl-acetate. All staining from bone marrow and blood was performed routinely according to standard procedures (20). The cytomorphologic diagnosis followed the criteria of the FAB classification and the new WHO classification (1, 3, 18).

### Example 4

### Cytogenetics

Chromosome analyses were performed on bone marrow or peripheral blood samples according to standard protocols (21). Metaphases were analyzed for G-bands using a modified GAG-banding technique as described elsewhere (22). Twenty to 25 metaphase cells were analyzed. The chromosomes were interpreted according to the International System for Human Cytogenetic Nomenclature (23).

### Example 4

### Fluorescence in situ Hybridization (FISH) on Interphase Nuclei

FISH was performed on interphase nuclei on bone marrow smears or on slides prepared for cytogenetic analysis. For interphase-FISH at least 100 interphase nuclei were evaluated. FISH was carried out using commercially available AML1-ETO, PML-RARA and CBFB probes (VYSIS, Downers Grove, II, USA). The signals were evaluated with an Axioskop^{R} (Zeiss, Jena, Germany). For documentation the analyzing system ISIS^{R} (MetaSystems, Altlussheim, Germany) was used.

### Example 4

### RNA Isolation and Reverse-Transcription-Polymerase-Chain-Reaction (RT-PCR)

Mononuclear cells were isolated by a Ficoll gradient separation. 1×10⁷ cells were lysed in RLT-buffer (Qiagen, Hilden, Germany) and total RNA was extracted with a RNeasy-kit (Qiagen) according to the manufacturers instructions. RNA was eluted in 50 μl of elution buffer.

Five μl of the total RNA, an equivalent quantity of 1×10⁶ cells or about 1 μg of RNA were reversely transcribed in a 40 μl reaction using 300 U of Superscript^{R} (Life Technologies, Karlsruhe, Germany) and random hexamers (Pharmacia, Freiburg, Germany).

PCR for the specific AML1-ETO, CBFB-MYH11, or PML-RARA fusion transcripts were performed as has been described (24). For each sample an ABL specific RT-PCR was performed to control the integrity of RNA using primers ABL5′: 5-GGCCAGTAGCATCTGACTTTG-3′ and ABL3′: 5′-ATGGTACCAGGAGTGTTTCTCC-3′. Strict precautions were taken to prevent contamination. Water instead of cDNA was included as a blank sample in each experiment. Amplification products were analyzed on 1.5% agarose gels stained with ethidium bromide.

### Example 4

### Microarray Experiments

For microarray analysis the GeneChip® System (Affymetrix, Santa Clara, Calif.) was used. The targets for GeneChip® analysis were prepared according to the current Expression Analysis Technical Manual. Briefly, lysates of the leukemia samples were homogenized (QIAshredder, Qiagen, Hilden, Germany) and total RNA extracted (RNeasy Mini Kit, Qiagen). Normally, 10 μg total RNA isolated from 1×10⁷ cells was used as starting material in the subsequent cDNA-synthesis using oligo[(dT)₂₄T7promotor]₆₅ primer (cDNA Synthesis System, Roche Diagnostics, Mannheim, Germany). The cDNA was purified by phenol:chlorophorm:IAA extraction (Ambion, Austin, Tex.) and acetate/ethanol precipitated over night. For detection of the hybridized target nucleic acid biotin-labeled ribonucleotides were incorporated during the in vitro transcription (Enzo® BioArray™ HighYield™ RNA Transcript Labeling Kit, ENZO, Farmingdale, USA). After quantification of the purified cRNA (RNeasy Mini Kit, Qiagen), 15 μg was fragmented by alkaline treatment (200 mM Tris-acetate, pH 8.2, 500 mM potassium acetate, 150 mM magnesium acetate) and added to the hybridization cocktail sufficient for 5 hybridizations on standard GeneChip® microarrays. Before hybridization onto U95Av2, Test3 microarrays (Affymetrix) were chosen for monitoring of the integrity of the cRNA. Washing and staining of the probe arrays were performed according to the current protocols (Micro_{—}1v1, EukGE-WS2v2). The Affymetrix software (Microarray Suite, Version 4.0.1) extracted fluorescence intensities from each element on the microarrays as detected by confocal laser scanning according to the manufacturers recommendations. Thirty-two out of 37 hybridization cocktails demonstrated high quality cRNA characteristics (Test3 probe arrays: 3′/5′ ratio of GAPDH probe sets ≦3.0) and were selected for building up class prediction models.

### Example 4

### Class Separation by Principal Component Analysis

Potential clusters corresponding to the genetic subgroups were visualized applying a two-step approach. The data were scaled from each array to a target intensity value 50 (Affymetrix Microarray Suite 4.0.1) in order to be able to perform inter-array comparisons. All data were permutated 100 cycles using the multiclass response parameter of the Significance Analysis of Microarrays algorithm (SAM) (25) (http://www-stat.stanford.edu/˜tibs/SAM/index.html). The total set of 12,600 genes was reduced to the significant differentially expressed genes. In a second step, the reduced set of genes was prepared for principal component analysis (PCA) and analyzed with J-Express (26) (http://www.molmine.com/). For visualization in a two-dimensional plot we chose the first two principal components as they captured most of the variation in the original data set.

### Example 4

### Class Prediction by Weighted Voting (13)

We adapted a previously described method to reduce the number of candidate genes that could distinguish between the three different cytogenetic AML subgroups (13). Briefly, to avoid division by zero or negative numbers as occurs due to the expression algorithm (Affymetrix Microarray Suite 4.0.1) we set all average fluorescence intensities of 1 or less to 1. Then, gene expression levels were log-transformed. Performing pairwise comparisons (A vs. B), for each gene g P(g,c) values and votes (defined by: P(g,c)=(m1(g)−m2(g))/(s1(g)+s2(g))) were calculated based on mean expression levels (m) and standard deviations (s) in the respective cytogenetic subgroup. Subsequently, votes were summed and prediction strength (PS) values reflected the margin of victory in the direction of either cytogenetic group A or B of the pairwise comparison. PS values range between 0 and 1, values >0.45 demonstrate significance (according to the permutation test). The relevance of selected genes was assessed by performing leave-one-out cross-validation. Only those genes that were contained in all cross validation classifiers were considered important. To determine a random association between genes we performed a permutation test (100 cycles). Because the number of informative genes, which are required to discriminate between samples, is unknown, we applied this method for different numbers of informative genes (range: 2 to 200). The minimal set of genes which provided optimal classification accuracy together with the highest prediction strength was selected to avoid overfitting. To visualize the identified genes and check their suitability for class separation a hierarchical cluster analysis was performed utilizing J-Express (26) (cluster method: average linkage; distance metric: euclidean). The accuracy of this class prediction model was validated on an independent test set of five cases of AML not fulfilling the cRNA high quality criterion as outlined above.

### Example 4

### Multiple-Tree Classifier

As basic units in this classifier, classification trees are used (27-29). The optimal number of trees has been determined to be 15 (data not shown). Class votes of these trees are aggregated by a vote-by-majority rule. The classifier was fed with gene expression intensity values from a set of 973 genes that had been chosen based on their r statistic:

$$r = \frac{{\sum }_{i = 1}^{k} \vert {μ}_{i} - \overline{μ} \vert}{{\sum }_{- i = 1}^{k} {σ}_{i}}$$

where μᵢ refers to the class averages, μ to the overall average, σᵢ to the within-class standard deviation, and summation is carried out over all k classes. The threshold was set to r>0.75. Classification trees were constructed as follows: tree building was performed while restricting trees to contain no more than n−1 nodes to discriminate between n classes. The C5.0 algorithm was used (28). The variables (gene expression intensities) used for tree construction were eliminated from the data set, and a new tree was calculated based on the truncated data set. This procedure was iterated until the predetermined number of trees had been reached. The accuracy of the multiple-tree classifier was estimated by 10-fold cross validation (30) and on an independent test set of data from 5 bone marrow aspirates, where the quality of the corresponding cRNA preparation was slightly lower than the high quality standards required for the training set.

### Example 4

### Results

### Example 4

### Characterization of Leukemia Samples

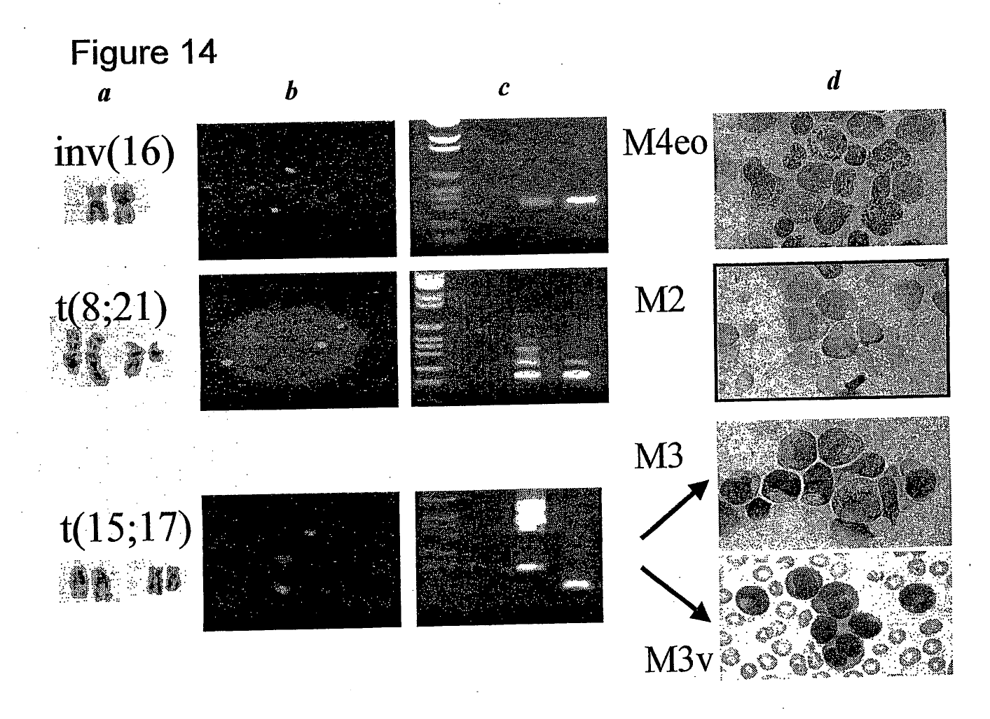

We investigated 37 AML cases representing three defined cytogenetic aberrations corresponding to four FAB subtypes: t(8;21)(q22;q22)/AML M2 (n=9), t(15;17)(q22;q12)/AML M3 or AML M3v (n=10, n=8), and inv(16)(p13q22)/AML M4eo (n=10). All cases were characterized by cytomorphology, cytogenetics, FISH, and RT-PCR (FIG. 14). All cases with AML and t(8;21) had AML M2, all with AML and inv(16) had AML M4eo, ten cases with AML and t(15;17) had AML M3, and eight cases with AML and t(15;17) had AML M3v. All patients showed these balanced abnormalities as the sole karyotype change. Using FISH analysis, more than 65% of cells demonstrated the specific signal constellation. The respective fusion transcripts were detected by RT-PCR in all samples. The median age of all patients was 53 years (range, 19-82 years; male:female=15:22) and did not differ between the respective groups. AML subtypes M3 and M3v both carry the same chromosomal aberration but differ in morphological aspects like nuclear configuration, granulation, and clinical aspects like white blood cell count (WBC). The median WBC count was 20,000/μl (range, 800-168,000/1) and was strikingly lower in patients with AML M3 as compared to all other patients (median, 6,200 vs. 36,500/μl, P=0.0002).

### Example 4

### Microarray Analyses

The gene expression profiles of 37 AML samples were evaluated. Thirty-two hybridization cocktails demonstrated high quality cRNA characteristics (Test3 probe arrays: 3′/5′ ratio of GAPDH probe sets <3.0) and were selected for building class prediction models: t(8;21)/AML M2 (n=7), t(15;17)/AML M3 or M3v (n=9, n=7), and inv(16)/AML M4eo (n=9). Five cases were primarily excluded (3′/5′ ratios ranging between 3.9 and 5.4, see Methods) and were used for subsequent validations of the class prediction models: t(8;21)/AML M2 (n=2), t(15;17)/AML M3 or M3v (n=1, n=1), and inv(16)/AML M4eo (n=1).

### Example 4

### Class Separation by Principal Component Analysis

In order to visualize clusters corresponding to the three underlying genetic subgroups we applied a two-step approach. Based on a permutation test (100 permutations) we correlated our expression data to the three different cytogenetic parameters (25). We obtained 1000 significant genes. By principal component analysis we were able to clearly separate the three distinct chromosomal aberrations t(8;21), t(15;17), and inv(16) (FIG. 15) (26). These data suggest that genetically defined AML subtypes can be specified and identified based on their gene expression profiles.

### Example 4

### Class Prediction by Weighted Voting (13)

In order to identify the genes which enable the accurate discrimination of these subgroups, we applied the data analysis methodology introduced by Golub et al. (13). We selected the minimal set of genes which provided optimal classification accuracy together with the highest prediction strength to avoid overfitting. Thirteen genes were sufficient to separate these AML subtypes with high precision (Table 24; Table 24 shows that a minimal set of 13 genes (GenBank accession numbers are given) is sufficient for accurate class prediction with optimal classification accuracy and highest prediction strength. Comparisons (A vs. B) were performed either between two distinct subtypes or between one distinct subtype and all other subtypes (=remainder), respectively. As calculated from pairwise comparisons, positive P(g,c) values indicate a higher expression in first class listed, negative P(g,c) values a higher expression in second class listed, respectively). GenBank accession numbers and detailed descriptions of the genes are given in table 25 (Table 25: Thirty-six genes separate accurately three distinct cytogenetic AML subtypes. GenBank accession numbers, approved human gene nomenclature symbol (*=not approved) and description of the function are presented. Six genes are included in the minimal set of both weighted voting according to Golub et al. (13) (total=13) and multiple-tree classifiers (total=29).

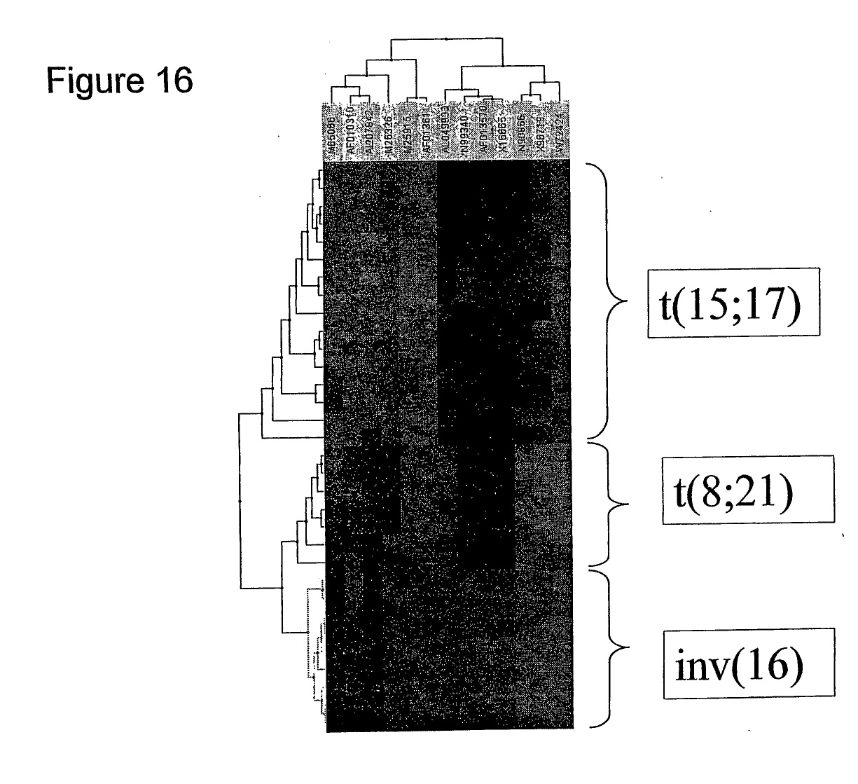

All 32 clinical samples could be assigned to their corresponding cytogenetic subtype with best accuracy in leave-one-out cross-validation (1.0). Prediction strength values ranged from 0.91 to 0.98 (Table 24). To illustrate these results we applied hierarchical clustering (31). The resulting dendrogram clearly demonstrates the capacity of this subset of genes to separate all AML cases according to their cytogenetic aberration (FIG. 16). This demonstrates that class prediction of a chromosomal aberration in AML is feasable solely based on gene expression data.

For external validation, we tested whether primarily excluded samples could also be accurately assigned to their specific cytogenetic category. Despite their non-optimal cRNA quality, all 5 cases were correctly classified with high prediction strength (0.76, 1.00, 1.00, 1.00, 1.00).

### Example 4

### Class Prediction by Multiple-Tree Models

As a second and independent methodological approach we developed a multiple-tree classifier to separate the three genetically defined subtypes based on the expression level of a minimal set of genes. In short, we computed classification trees to discriminate between the different AML subclasses. To avoid overfitting of a singular tree model, we computed a multiple-tree model using an iteratively reduced set of genes. For each tree, we used only those genes that have not been used by the previously computed classification tree. The procedure is stopped when a predetermined number of trees has been reached. For this study, the optimal number of trees was calculated to be 15. The votes of the 15 trees were aggregated by a vote-by-majority rule. Equal votes for two of the three classes were counted as misclassification.

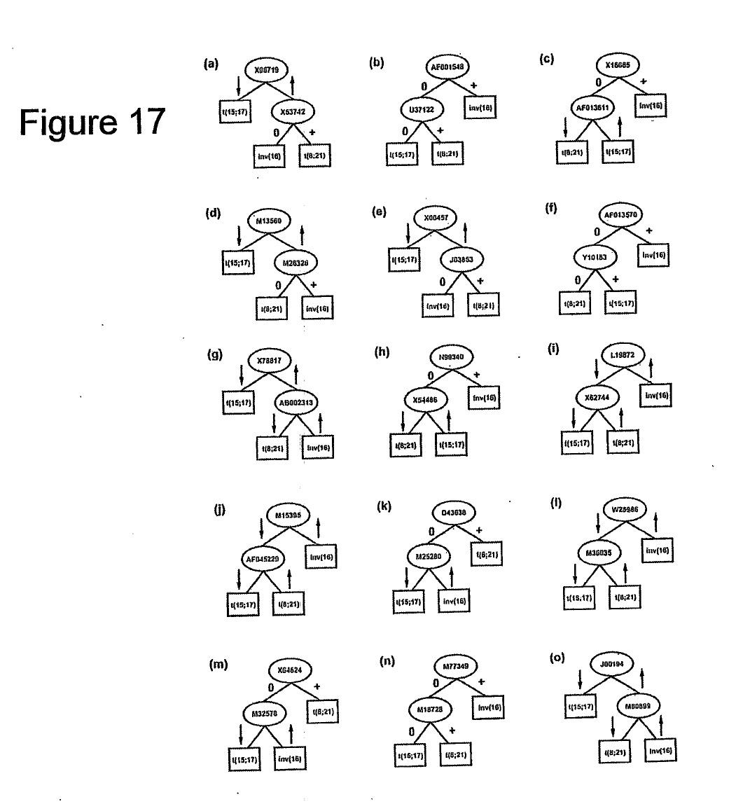

The classifier utilized the expression values of 29 genes (MYH11 was identified twice by two different probe sets; Table 25) to discriminate between three classes, namely samples displaying t(15;17), t(8;21), and inv(16) (FIG. 17). The accuracy on the training set (n=32) was 100%, and on the independent test set (n=5) 100%. The average accuracy in ten-fold cross validation was 94%.

In summary, we identified 36 genes using two independent methodologies for class prediction in AML (Table 25). Six genes were described in both calculations, seven were found exclusively in the minimal set according to Golub et al. (13), and another 23 genes using multiple-tree classifiers.

### Example 4

### Correlation of Phenotype and Gene Expression Profile

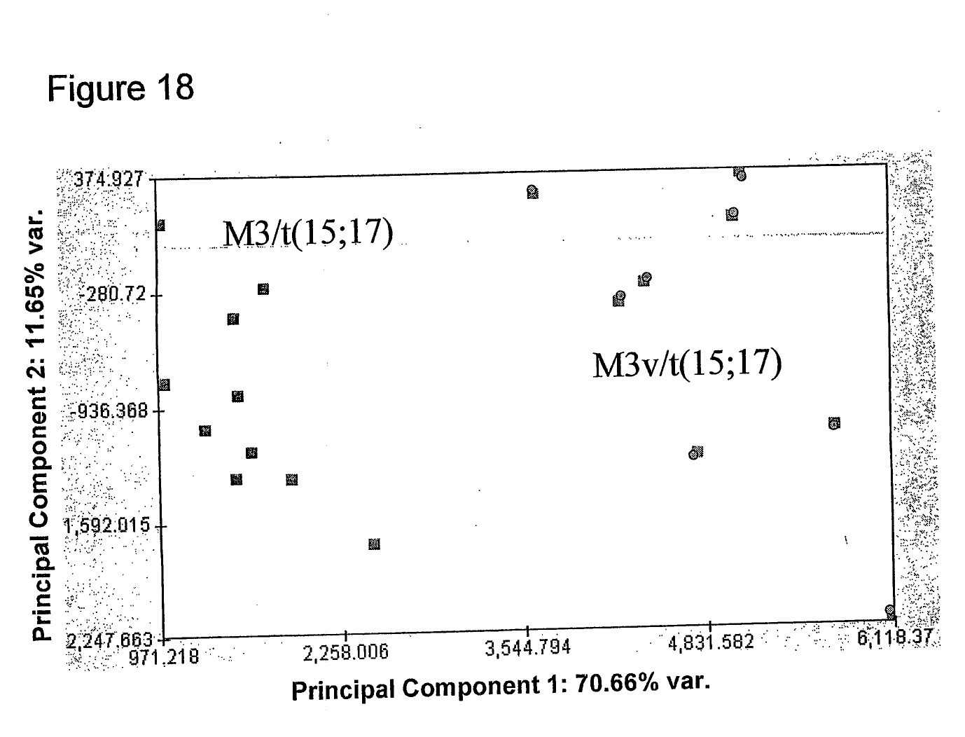

We were able to demonstrate striking correlations between genotype and gene expression profiles in three genetically defined subgroups of AML. In addition, we answered the question, whether the cytogenetically identical AML with t(15;17) but appearing with two different phenotypes, AML M3 or AML M3v (FIG. 14), can also be separated by different gene expression patterns. We used 100-fold permutation of M3 (n=10) and M3v (n=8) data followed by principal component analysis and hierarchical cluster analysis based on 82 informative genes (data not shown). Separation into the corresponding two morphologically defined FAB subtypes M3 and M3v was possible in all cases (FIG. 18) and suggests also a close correlation between phenotype and gene expression profile.

### Example 4

### Discussion

This is the first study to demonstrate an unequivocal association between disease-specific genetic alterations and distinct gene expression profiles. For each of the three analyzed clearly defined subtypes of AML (t(8;21), t(15;17), inv(16)) patterns of gene expression were identified that were homogeneous within all samples of the respective subgroups but clearly differed between these three subgroups. The analyzed samples represent disease subtypes that are specifically defined on the genetic and the phenotypic level by conventional diagnostics including cytomorphology, cytogenetics, and molecular genetics.

By applying two independent approaches for the analysis of microarray data, the present study demonstrates that AML samples from previously defined subtypes (3) can be classified adequately on the basis of gene expression profiles. It is intriguing that there is both sufficient coherence in gene expression within and difference between these subtypes to classify them with high accuracy even though the samples derive from the same myeloid cell lineage.

In order to correlate gene expression with cytogenetics Virtaneva et al. compared the expression status of 6,606 genes of AML blasts with normal cytogenetics and trisomy 8 as the sole abnormality. While in this study normal CD34+ cells clustered into a distinct group, AML with trisomy 8 and AML with normal karyotype intercalated with each other. Microarray analyses showed an overall increased gene expression of genes located on chromosome 8 suggesting a gene-dosage effect(14). AML with trisomy 8 is heterogeneous on the phenotypic level as it occurs in different FAB subtypes. In contrast, AML with t(15;17), inv(16) and t(8;21) show a very close correlation to distinct morphological subtypes. Furthermore, trisomy 8 is probably not a primary, disease-defining aberration leading to AML as it also occurs in addition to a variety of different cytogenetic and molecular genetic abnormalities (32, 33). In contrast to this study, Armstrong et al. compared samples of the more homogeneous group of ALL with MLL translocations to ALL without MLL translocations and to AML (15). They demonstrated that ALL with MLL translocations comprises a distinct disease which can be classified robustly by gene expression profiling.

The main focus of the present analyses was the assessment of the differences between three highly characterized subgroups of AML defined by specific primary chromosome aberrations. As anticipated, it was shown that AML with t(8;21) and AML with inv(16), which both involve alterations of the core binding factor-complex, are more related to each other as compared to AML with t(15;17) (34). Both phenotypically different subtypes of AML with t(15;17), AML M3 and AML M3v, cluster within one area. In an additional analysis, also the latter two subtypes were separated from each other based on their gene expression profiles. This data suggests the existence of further genetic and not yet identified alterations leading to the different phenotypes of AML M3 and AML M3v. One possible candidate gene is FLT3 which is mutated more frequently in AML M3v than in AML M3 (67% vs. 19%, P=0.001) (35).

Several studies confirmed that gene expression profiles can be used for class prediction. This has been shown for acute leukemias, round blue cell tumors, and malignant melanomas (13, 36-38) as well as for different types of solid tumors using multi-class cancer classification (39). While the selection of different subgroups in these studies was performed using exclusively phenotypic criteria, other studies were based on genetically defined entities (40, 41). In the present study not only the discrimination of the three genetically defined AML subgroups was accomplished but also all these cases of AML were separated from normal bone marrow (data not shown) (42).

To develop a classifier two independent approaches were applied. While classification by weighted voting according to Golub et al. (13) allows the discrimination between the three classes based on a minimal set of 13 genes, the multiple-tree classifier utilizes 30 genes. As indicated by cross-validation, generalization properties are excellent for the multiple-tree classifier, i.e. it is likely to perform equally well on new, unseen samples. Furthermore, it can be easily extended to more than the three subclasses described in the present study.

Our classifiers contained genes already known to be primarily involved in the pathogenesis of the respective entities, namely MYH11(43) and ETO(44).

Presumably, the detection of overexpression of MYH11 in inv(16) cases and of ETO in t(8;21) cases relates to the detection of the fusion gene transcripts rather than of the wild type transcripts. The other genes identified belong to various functional categories. Their potential pathogenetic significance in AML has to be clarified yet.

It is expected that the extension of the present analyses to currently less well-defined AML will identify additional subgroups of AML with clinical relevance based on their gene expression profiles. The feasibility of such an approach has been demonstrated for the first time for diffuse large B-cell lymphoma (45). Alizadeh et al. have subdivided an entity previously considered homogeneous by various pathological methods into two not only new but also prognostically highly relevant subgroups. In two recent studies, gene expression profiling also in breast cancer revealed subgroups significantly differing in their prognosis (46, 47). With regard to AML, this approach may be most promising in AML with normal karyotype. This subgroup cannot be further defined on the cytogenetic level and is characterized by an Intermediate prognosis possibly masking poor and favorable subgroups.

In addition, the current data may have major implications with regard to delineating aberrant gene expression pathways underlying the pathogenesis of AML. As has been shown in mantle cell lymphoma and medulloblastoma (48, 49) the extension of our analyses to all subgroups of AML should enable us to define the deregulated genes important for the initiation and the progression of AML. Finally, these analyses will promote the identification of new targets for specific treatment approaches.

### Example 4

### REFERENCES

1. Bennett, J. M., Catovsky, D., Daniel, M. T., Flandrin, G., Galton, D. A., Gralnick, H. R. & Sultan, C. (1976) Br. J. Haematol. 33, 451-458.
2. Harris, N. L., Jaffe, E. S., Diebold, J., Flandrin, G., Muller Hermelink, H. K., Vardiman, J., Lister, T. A. & Bloomfield, C. D. (1999) J. Clin. Oncol. 17, 3835-3849.
3. Jaffe, E. S., Harris, N. L., Stein, H. & Vardiman, J. W. World Health Organization Classification of Tumours. Pathology and Genetics of Tumours of Haematopoietic and Lymphoid Tissues. 2001. Lyon, IARC Press.
4. Grimwade, D., Walker, H., Oliver, F., Wheatley, K., Harrison, C., Harrison, G., Rees, J., Hann, I., Stevens, R., Burnett, A. et al. (1998) Blood 92, 2322-2333.
5. Bloomfield, C. D., Shuma, C., Regal, L., Philip, P. P., Hossfeld, D. K., Hagemeijer, A. M., Garson, O. M., Peterson, B. A., Sakurai, M., Alimena, G. et al. (1997) Cancer 80, 2191-2198.
6. Buchner, T., Hiddemann, W., Wormann, B., Loffler, H., Gassmann, W., Haferlach, T., Fonatsch, C., Haase, D., Schoch, C., Hossfeld, D. et al. (1999) Blood 93, 4116-4124.
7. Schoch, C., Haferlach, T., Haase, D., Fonatsch, C., Loffler, H., Schlegelberger, B., Staib, P., Sauerland, M. C., Heinecke, A., Buchner, T. et al. (2001) Br. J. Haematol. 112, 118-126.
8. Schoch, C., Kern, W., Krawitz, P., Dugas, M., Schnittger, S., Haferlach, T. & Hiddemann, W. (2001) Blood 98, 3500.
9. Pabst, T., Mueller, B. U., Harakawa, N., Schoch, C., Haferlach, T., Behre, G., Hiddemann, W., Zhang, D. E. & Tenen, D. G. (2001) Nat. Med. 7, 444-451.
10. Yuan, Y., Zhou, L., Miyamoto, T., Iwasaki, H., Harakawa, N., Hetherington, C. J., Burel, S. A., Lagasse, E., Weissman, I. L., Akashi, K. et al. (2001) Proc. Natl. Acad. Sci. U.S. A 98, 10398-10403.
11. Castilla, L. H., Wijmenga, C., Wang, Q., Stacy, T., Speck, N. A., Eckhaus, M., Marin Padilla, M., Collins, F. S., Wynshaw Boris, A. & Liu, P. P. (1996) Cell 87, 687-696.
12. Brown, D., Kogan, S., Lagasse, E., Weissman, I., Alcalay, M., Pelicci, P. G., Atwater, S. & Bishop, J. M. (1997) Proc. Natl. Acad. Sci. U.S.A. 94, 2551-2556.
13. Golub, T. R., Slonim, D. K., Tamayo, P., Huard, C., Gaasenbeek, M., Mesirov, J. P., Coller, H., Loh, M. L., Downing, J. R., Caligiuri, M. A. et al. (1999) Science 286, 531-537.
14. Virtaneva, K., Wright, F. A., Tanner, S. M., Yuan, B., Lemon, W. J., Caligiuri, M. A., Bloomfield, C. D., de la Chapelle, A. & Krahe, R. (2001) Proc. Natl. Acad. Sci. U.S. A 98, 1124-1129.
15. Armstrong, S. A., Staunton, J. E., Silverman, L. B., Pieters, R., den Boer, M. L., Minden, M. D., Sallan, S. E., Lander, E. S., Golub, T. R. & Korsmeyer, S. J. (2002) Nat. Genet. 30, 41-47.
16. Haferlach, T., Bennett, J. M., Loffier, H., Gassmann, W., Andersen, J. W., Tuzuner, N., Cassileth, P. A., Fonatsch, C., Schoch, C., Schlegelberger, B. et al. (1996) Leuk. Lymphoma. 23, 227-234.
17. Haferlach, T., Winkemann, M., Loffler, H., Schoch, R., Gassmann, W., Fonatsch; C., Schoch, C., Poetsch, M., Weber Matthiesen, K. & Schlegelberger, B. (1996) Blood 87, 2459-2463.
18. Bennett, J. M., Catovsky, D., Daniel, M. T., Flandrin, G., Galton, D. A., Gralnick, H. R. & Sultan, C. (1985) Ann. Intern. Med. 103, 620-625.
19. Dugas, M., Schoch, C., Schnittger, S., Haferlach, T., Danhauser-Riedl, S., Hiddemann, W., Messerer, D. & Uberla, K. (2001) Leukemia 15, 1805-1810.
20. Loffler, H. & Rastetter, J. Atlas of clinical hematology. 1999. Berlin, Springer. Ref Type: Serial (Book, Monograph)
21. Stollmann, B., Fonatsch, C. & Havers, W. (1985) Br. J. Haematol. 60, 183-196.
22. Fonatsch, C., Schaadt, M., Kirchner, H. & Diehl, V. (1980) Int. J. Cancer 26, 749-756.
23. ISCN 1995, Guidelines for Cancer Cytogenetics, Supplement to: An International System for Human Cytogenetic Nomenclature. ed. Mitelman, F. 1995. S. Karger.

24. Evans, P., Jack, A., Short, M., Haynes, A., Shiach, C., Owen, R., Johnson, R. & Morgan, G. J. (1995) Leukemia 9, 1285-1286.
25. Tusher, V. G., Tibshirani, R. & Chu, G. (2001) Proc. Natl. Acad. Sci. U.S. A 98, 5116-5121.
26. Dysvik, B. & Jonassen, I. (2001) Bioinformatics. 17, 369-370.
27. Breiman, L., Friedman, J. H., Olshen, R. A. & Stone, C. J. Classification and regression trees. 1984. Monterey, Wadsworth & Brooks.
28. Quinlan, J. R. Programs for machine learning. 1993. San Mateo, Morgan Kaufmann.
29. Berrar, D., Granzow, M., Dubitzky, W., Lichter, P. & Eils, R. New insights in clinical impact of molecular genetic data by knowledge-driven data mining. 275-281. 2001. Wisconsin, Omnipress. Proceedings of the Second International Conference on Systems Biology.
30. Efron, B. & Tibshirani, R. J. An introduction to the bootstrap. 237-257. 1981. London/New York, Chapman & Hall.
31. Eisen, M. B., Spellman, P. T., Brown, P. O. & Botstein, D. (1998) Proc. Natl. Acad. Sci. U.S. A 95, 14863-14868.
32. Schoch, C., Haase, D., Fonatsch, C., Haferlach, T., Loffler, H., Schlegelberger, B., Hossfeld, D. K., Becher, R., Sauerland, M. C., Heinecke, A. et al. (1997) Br. J. Haematol. 99, 605-611.
33. Schnittger, S., Kinkelin, U., Schoch, C., Heinecke, A., Haase, D., Haferlach, T., Buchner, T., Wormann, B., Hiddemann, W. & Griesinger, F. (2000) Leukemia 14, 796-804.
34. Friedman, A. D. (1999) Leukemia 13, 1932-1942.
35. Schnittger, S., Schoch, C., Dugas, M., Kern, W., Staib, P., Wuchter, C., Loffier, H., Sauerland, M. C., Serve, H., Buchner, T. et al. (2002) Blood, in press.

36. Miyazato, A., Ueno, S., Ohmine, K., Ueda, M., Yoshida, K., Yamashita, Y., Kaneko, T., Mori, M., Kirito, K., Toshima, M. et al. (2001) Blood 98, 422-427.
37. Khan, J., Wei, J. S., Ringner, M., Saal, L. H., Ladanyi, M., Westermann, F., Berthold, F., Schwab, M., Antonescu, C. R., Peterson, C. et al. (2001) Nat. Med. 7, 673-679.
38. Bittner, M., Meltzer, P., Chen, Y., Jiang, Y., Seftor, E., Hendrix, M., Radmacher, M., Simon, R., Yakhini, Z., Ben Dor, A. et al. (2000) Nature 406, 536-540.
39. Ramaswamy, S., Tamayo, P., Rifkin, R., Mukherjee, S., Yeang, C. H., Angelo, M., Ladd, C., Reich, M., Latulippe, E., Mesirov, J. P. et al. (2001) Proc. Natl. Acad. Sci. U.S. A 98, 15149-15154.
40. Hedenfalk, I., Duggan, D., Chen, Y., Radmacher, M., Bittner, M., Simon, R., Meltzer, P., Gusterson, B., Esteller, M., Kallioniemi, O. P. et al. (2001) N. Engl. J. Med. 344, 539-548.
41. Perou, C. M., Sorlie, T., Eisen, M. B., van de, R. M., Jeffrey, S. S., Rees, C. A., Pollack, J. R., Ross, D. T., Johnsen, H., Akslen, L. A. et al. (2000) Nature 406, 747-752.
42. Kohlmann, A., Dugas, M., Schoch, C., Schnittger, S., Mergenthaler, S., Kern, W., Haferlach, T. & Hiddemann, W. (2001) Blood 98, 91a.
43. Miller, J. D., Stacy, T., Liu, P. P. & Speck, N. A. (2001) Blood 97, 2248-2256.
44. Gelmetti, V., Zhang, J., Fanelli, M., Minucci, S., Pelicci, P. G. & Lazar, M. A. (1998) Mol. Cell. Biol. 18, 7185-7191.
45. Alizadeh, A. A., Eisen, M. B., Davis, R. E., Ma, C., Lossos, I. S., Rosenwald, A., Boldrick, J. C., Sabet, H., Tran, T., Yu, X. et al. (2000) Nature 403, 503-511.
46. Sorlie, T., Perou, C. M., Tibshirani, R., Aas, T., Geisler, S., Johnsen, H., Hastie, T., Eisen, M. B., van de, R. M., Jeffrey, S. S. et al. (2001) Proc. Natl. Acad. Sci. U.S. A 98, 10869-10874.
47. West, M., Blanchette, C., Dressman, H., Huang, E., Ishida, S., Spang, R., Zuzan, H., Olson, J. A., Jr., Marks, J. R. & Nevins, J. R. (2001) Proc. Natl. Acad. Sci. U.S. A 98, 11462-11467.
48. Hofmann, W. K., de Vos, S., Tsukasaki, K., Wachsman, W., Pinkus, G. S., Said, J. W. & Koeffler, H. P. (2001) Blood 98, 787-794.
49. MacDonald, T. J., Brown, K. M., LaFleur, B., Peterson, K., Lawlor, C., Chen, Y., Packer, R. J., Cogen, P. & Stephan, D. A. (2001) Nat. Genet. 29, 143-152.

### Example 6

### Correlation of Protein Expression and Gene Expression in Acute Myeloid Leukemia

Introduction

The determination of the surface and cytoplasmic expression of characteristic proteins by flow cytometry (FC) is a common method applied to the diagnosis and the subclassification of acute myeloid leukemias (AML)¹. The oligonucleotide microarray analysis (MA) represents a novel technology for the simultaneous detection of the mRNA abundance of large numbers of genes^{2,3}. Based on specific gene-expression patterns distinct disease entities have been identified⁴⁻⁶. Therefore MA may become of major importance as a diagnostic tool for AML in the near future^{7,8}. However, up to now data on the correlation between protein expression levels and mRNA abundance are limited⁹⁻¹². To analyze the relation of protein expression and mRNA abundance in AML we performed 450 individual comparisons of 29 genes in 25 patients with AML at diagnosis analyzed by FC and MA in parallel¹³.

Methods

Samples

Bone marrow samples from highly characterized patients with newly diagnosed and untreated AML were used. Samples had been analyzed by cytomorphology, cytochemistry, cytogenetics and molecular genetics in all cases and were characterized by either of the balanced chromosomal aberrations t(8;21), t(15;17), or inv(16) and the respective molecular and morphologic features⁷. The studies abide by the rules of the local Internal Review Board and the tenets of the revised Helsinki protocol.

Flow Cytometry

The studies were performed on cells isolated from bone marrow by Ficoll-Hypaque density gradient centrifugation as described previously¹⁴. Applying triple-stainings and isotype controls monoclonal antibodies against 29 antigens were used in the following combinations as designed for diagnostic purposes (conjugated with the fluorochromes FITC, PE, and PC-5, respectively): CD34/CD2/CD33, CD7/CD33/CD34, CD34/CD56/CD33, CD11b/CD33/CD34, CD64*/CD4/CD45, CD15*/CD13/CD33, HLA-DR/CD33/CD34, CD34/CD135/CD33, CD34/CD116/CD33, CD34/NG2/CD33, CD38/CD133**/CD34, CD61/CD14/CD45, CD36/CD235a/CD45, CD34/CD10/CD19, MPO***/LF***/cYCD15, TdT/cyCD22/cyCD3, TdT/cyCD79a/cyCD3. All antibodies were purchased from Immunotech (Marseilles, France), except for: *=Medarex (Annandale, N.J.); **=Milteny Biotech (Bergisch Gladbach, Germany); ***=Caltag (Burlingame, Calif.). The respective combinations of antibodies were added to 1×10⁶ cells (volume, 100 μl) and incubated for ten minutes at room temperature. The samples were then washed twice in phosphate-buffered saline (PBS) and resuspended in 0.5 ml PBS. FC analysis was performed using a FACSCalibur flow cytometer (Becton Dickinson, San Jose, Calif.). Analysis of list-mode files was performed by means of the CellQuest Pro Software (Becton Dickinson). Antigen expression was rated positive at a cut-off level of 20% of the cells within the mononuclear gate for membrane proteins and at a cut-off level of 10% for cytoplasmic antigens. Mean fluorescence intensity values were calculated for all events with fluorescence values higher than isotype controls.

Microarray Experiments

For microarray analysis the GeneChip® System (Affymetrix, Santa Clara, Calif.) was used. The targets for GeneChip® analysis were prepared according to the current Expression Analysis Technical Manual. Briefly, lysates of the leukemia samples were homogenized (QIAshredder, Qiagen, Hilden, Germany) and total RNA extracted (RNeasy Mini Kit, Qiagen). Normally, 10 μg total RNA isolated from 1×10⁷ cells were used as starting material in the subsequent cDNA-synthesis using oligo[(dT)₂₄T7promotor]⁶⁵ primer (cDNA Synthesis System, Roche Diagnostics, Mannheim, Germany). The cDNA was purified by phenol:chlorophorm:isoamylalcohol extraction (Ambion, Austin, Tex.) and acetate/ethanol precipitated overnight. For detection of the hybridized target nucleic acid biotin-labeled ribonucleotides were incorporated during the in vitro transcription (Enzo® BioArray™ HighYield™ RNA Transcript Labeling Kit, ENZO, Farmingdale, USA). After quantification of the purified cRNA (RNeasy Mini Kit, Qiagen), 15 μg were fragmented by alkaline treatment (200 mM Tris-acetate, pH 8.2, 500 mM potassium acetate, 150 mM magnesium acetate) and added to the hybridization cocktail sufficient for 5 hybridizations on standard GeneChip® microarrays. Before hybridization onto U95Av2, Test3 microarrays (Affymetrix) were chosen for monitoring of labelling efficiency and the integrity of the cRNA. Washing and staining of the probe arrays was performed according to the current protocols (Micro_{—}1v1, EukGE-WS2v4). The Affymetrix software (Microarray Suite, Version 4.0.1) extracted, fluorescence intensities from each element on the microarrays as detected by confocal laser scanning according to the manufacturers recommendations. In order to be able to compare different experiments the global

microarray intensities were scaled to a common target intensity. Furthermore, the mRNA abundance of the genes was qualitatively rated as a) present, b) marginal, and c) absent calls, respectively.

Statistics

A total of 29 genes were analyzed in 25 patients with AML. The congruence of positivity and negativity of the expression of the respective genes as determined by FC and MA was analyzed for each gene in each individual patient. Comparisons of microarray intensities were performed by Mann-Whitney U-test. Analyses for bivariate correlations of mRNA and protein expression levels were performed by Pearson's correlation using SPSS, Version 10.0.7.

Results and Discussion

Twenty-five cases of AML were analyzed in parallel by FC and MA for the expression of 29 genes. Seven had AML M2 with t(8;21), 5 had AML M3 with t(15;17), 7 had AML M3v with t(15;17), and 6 had AML M4Eo with inv(16). A total of 450 comparisons of individual expression data obtained by both methods were performed. Of these, 399 (88.7%) revealed congruent results for protein expression and mRNA abundance (230 cases (51.1%) with positive expression and 169 cases (37.6%) with negative expression, respectively; table 26). In 30 comparisons (6.7%) MA detected positivity for mRNA expression (call: present) while the results of FC indicated negativity. In 21 cases (4.7%) protein expression was demonstrated by FC while no mRNA expression was detected by MA (call: absent).

Focussing on the genes most specific for the diagnosis of AML, i.e. myeloperoxidase, CD13, and CD33, a high correlation between protein expression and mRNA abundance was observed (congruence in 73 of 75 comparisons (97%)). In detail, all cases were rated positive for expression of myeloperoxidase and all but one were positive for both CD13 and CD33, respectively, by both methods. Furthermore, for most other genes essential for the subclassification of AML as well as for the distinction of AML from acute lymphoblastic leukemia and chronic leukemias the results obtained by both methods were always congruent (i.e., for CD10, CD22, CD7, CD133, CD116, CD11b, CD61, CD45, HLA-DR, NG2) or were congruent in the majority (117/140, 84%) of cases (CD79a, CD19, CD2, CD3, CD15, Lactoferrin, CD14, CD235a, CD135, CD34; Table 26).

Furthermore, the high correlations between protein expression and mRNA abundance were not limited to congruence in positivity but were significantly correlated also quantitatively. To proof this, the protein expression levels and mRNA abundance were compared by Pearson's correlation in genes expressed in the majority of the analyzed cases. These comparisons revealed significant correlations for the fluorescence intensities as assessed by FC and MA for CD13 (p=0.001), CD33 (p=0.034), CD34 (p=0.003), CD45 (p=0.015), CD15 (p=0.016), and CD7 (p=0.033) and thus further underline the high coherence of expression patterns for both protein and mRNA (FIG. 19).

Thirty comparisons displayed mRNA expression and no protein expression. Due to the ongoing process of maturation (CD14, CD15) and due to the cross-lineage expression of the genes (CD3, CD19) the levels of mRNA abundance may have been to low to result in detectable protein expression levels using the described cut-off levels of 20% and 10%, respectively. This suggestion is supported by a quantitative analysis of mRNA expression data which shows relatively low albeit positive levels for the respective cases and genes (mean average fluorescence intensity, 46.7±54.5 in cases positive for CD14, CD15, CD3, or CD19 versus 389.4±831.0 in all positive cases, Mann-Whitney U-test: p<0.001) while at the same time protein expression amounts to a mean of 5±4%.

Twenty-one comparisons displayed positivity by FC and negativity of MA, which comprise 4.7% of all individual comparisons performed. These discrepancies most probably are due to: a) erythrocytic debris positive for CD36 interfering with the acquisition of CD36 negative cells during flow cytometric analysis; b) differences between both methods in the selected DNA sequences and antigen epitopes, respectively, detected (i.e. CD38, CD4, CD56); and c) differences in the stability of mRNA and protein of the respective genes.

Overall, these results demonstrate for the first time that there is a significant correlation between protein expression and gene expression in AML and that the antigens so far identified essential for the diagnosis and subclassification of AML by flow cytometry may represent additional candidate genes when using MA as a diagnostic tool for molecular cancer class prediction^{15,16}. Furthermore, it is anticipated that the present analyses represent a prime example and will be reproduced for a variety of other entities like lymphoid malignancies. Due to their high potential to assess the expression patterns of high numbers of genes and due to their excellent reproducibility features microarrays are a promising future diagnostic tool. As a consequence, they may replace the more time and resource consuming diagnostic methods currently used for diagnosing leukemias like cytomorphology, cytogenetics, and FC.

### REFERENCES FOR EXAMPLE 5

1. Weir, E. G. and Borowitz, M. J. Flow cytometry in the diagnosis of acute leukemia. Semin. Hematol., 38:124-138, 2001.
2. Liotta, L. and Petricoin, E. Molecular profiling of human cancer. Nat. Rev. Genet., 1: 48-56, 2000.
3. Kohlmann, A., Dugas, M., Schoch, C., Schnittger, S., Mergenthaler, S., Kern, W., Haferlach, T., and Hiddemann, W. Gene expression profiles of distinct AML subtypes in comparison to normal bone marrow. Blood, 98: 91a, 2001.
4. Alizadeh, A. A., Eisen, M. B., Davis, R. E., Ma, C., Lossos, I. S., Rosenwald, A., Boldrick, J. C., Sabet, H., Tran, T., Yu, X., Powell, J. I., Yang, L., Marti, G. E., Moore, T., Hudson, J., Jr., Lu, L., Lewis, D. B., Tibshirani, R., Sherlock, G., Chan, W. C., Greiner, T. C., Weisenburger, D. D., Armitages, J. O., Warnke, R., Staudt, L. M., and Distinct types of diffuse large B-cell lymphoma identified by gene expression profiling. Nature, 403: 503-511, 2000.
5. Hedenfalk, I., Duggan, D., Chen, Y., Radmacher, M., Bittner, M., Simon, R., Meltzer, P., Gusterson, B., Esteller, M., Kallioniemi, O. P., Wilfond, B., Borg, A., and Trent, J. Gene-expression profiles in hereditary breast cancer. N. Engl. J. Med., 344: 539-548, 2001.
6. Khan, J., Wei, J. S., Ringner, M., Saal, L. H., Ladanyi, M., Westermann, F., Berthold, F., Schwab, M., Antonescu, C. R., Peterson, C., and Meltzer, P. S. Classification and diagnostic prediction of cancers using gene expression profiling and artificial neural networks. Nat. Med., 7: 673-679, 2001.
7. Schoch, C., Kohlmann, A., Schnittger, S., Mergenthaler, S., Dugas, M., Kern, W., Hiddemann, W., and Haferlach, T. Specific abnormalities on the genomic level result in a distinct gene expression pattern detected by oligonucleotide microarrays: an analysis of 25 patients with AML M2/t(8;21), AML M3/M3v/t(15;17) and AML M4eo/inv(16). Blood, 98: 92a, 2002.
8. Armstrong, S. A., Staunton, J. E., Silverman, L. B., Pieters, R., den Boer, M. L., Minden, M. D., Sallan, S. E., Lander, E. S., Golub, T. R., and Korsmeyer, S. J. MLL translocations specify a distinct gene expression profile that distinguishes a unique leukemia. Nat. Genet., 30: 41-47, 2002.
9. Ahram, M., Best, C. J., Flaig, M. J., Gillespie, J. W., Leiva, I. M., Chuaqui, R. F., Zhou, G., Shu, H., Duray, P. H., Linehan, W. M., Raffeld, M., Ornstein, D. K., Zhao, Y., Petricoin, E. F., III, and Emmert-Buck, M. R. Proteomic analysis of human prostate cancer. Mol. Carcinog., 33: 9-15, 2002.
10. Serrano, J., Roman, J., Jimenez, A., Castillejo, J. A., Navarro, J. A., Sanchez, J., Garcia-Castellanos, J. M., Martin; C., Maldonado, J., and Torres, A. Genetic, phenotypic and clinical features of acute lymphoblastic leukemias expressing myeloperoxidase mRNA detected by RT-PCR. Leukemia, 13: 175-180, 1999.
11. Zhan, F., Hardin, J., Kordsmeier, B., Bumm, K., Zheng, M., Tian, E., Sanderson, R., Yang, Y., Wilson, C., Zangari, M., Anaissie, E., Morris, C., Muwalla, F., van Rhee, F., Fassas, A., Crowley, J., Tricot, G., Barlogie, B., and Shaughnessy, J., Jr. Global gene expression profiling of multiple myeloma, monoclonal gammopathy of undetermined significance, and normal bone marrow plasma cells. Blood, 99: 1745-1757, 2002.
12. Falini, B. and Mason, D. Y. Proteins encoded by genes involved in chromosomal alterations in lymphoma and leukemia: clinical value of their detection by immunocytochemistry. Blood, 99: 409-426, 2002.
13. Kern, W., Kohlmann, A., Wuchter, C., Schnittger, S., Schoch, C., Mergenthaler, S., Ratei, R., Ludwig, W. D., Hiddemann, W., and Haferlach, T. Correlation of gene expression and protein expression in acute myeloid leukemia as assessed by microarray analysis and flow cytometry. Blood, 98: 92a, 2001.
14. Wuchter, C., Harbott, J., Schoch, C., Schnittger, S., Borkhardt, A., Karawajew, L., Ratei, R., Ruppert, V., Haferlach, T., Creutzig, U., Dorken, B., and Ludwig, W. D. Detection of acute leukemia cells with mixed lineage leukemia (MLL) gene rearrangements by flow cytometry using monoclonal antibody 7.1. Leukemia, 14: 1232-1238, 2000.
15. Golub, T. R., Slonim, D. K., Tamayo, P., Huard, C., Gaasenbeek, M., Mesirov, J. P., Coller, H., Loh, M. L., Downing, J. R., Caligiuri, M. A., Bloomfield, C. D., and Lander, E. S. Molecular classification of cancer: class discovery and class prediction by gene expression monitoring. Science, 286: 531-537, 1999.
16. Ramaswamy, S., Tamayo, P., Rifkin, R., Mukherjee, S., Yeang, C. H., Angelo, M., Ladd, C., Reich, M., Latulippe, E., Mesirov, J. P., Poggio, T., Gerald, W., Loda, M., Lander, E. S., and Golub, T. R. Multiclass cancer diagnosis using tumor gene expression signatures. Proc. Natl. Acad. Sci. U.S.A, 98: 15149-15154, 2001.

### Example 6

### Gene Expression Profiles of Distinct Cytogenetic AML Subtypes as Defined by the New WHO Classification: A Study of 45 Patients

### Example 6

### Introduction

Since their introduction, microarrays have been promising tools for basic research. With regard to leukemia, the pivotal discrimination of unselected acute lymphoblastic (ALL), and acute myeloid leukemia (AML) samples based on their gene expression signatures inspired numerous studies (Golub et al., 1999). We performed gene expression analyses to designate candidate genes for discriminating specific AML samples from normal bone marrow (BM) of healthy volunteers. With regard to the classification of hematological malignancies according to the WHO, distinct AML subtypes have been established based on genetic abnormalities of the leukemic blasts. Here, we demonstrate gene expression analyses of 8 healthy BM donors and 45 leukemia patients representing four cytogenetic subtypes of AML: t(8;21)(q22;q22), inv(16)(p13q22), t(15;17)(q22;q12), and t(11q23)/MLL. Combining different approaches for data analysis a minimal set of genes was identified to designate a reliable class prediction model. Based on the expression pattern of 39 genes, cytogenetically defined AML subtypes could accurately be predicted and separated from healthy BM. Taken together, gene expression signatures of AML cases with recurrent genetic abnormalities demonstrate a very close correlation between genotype and gene expression. Therefore, introducing a set of candidate genes, expression profiling may serve for diagnosis of AML subtypes defined by the new WHO classification.

### Example 6

### Material and Methods

We analyzed BM aspirates from 8 healthy volunteers and the following 45 untreated AML patients:
t(8;21)(q22;q22)/AML M2 (n=9),
t(15;17)(q22;q12)/AML M3/M3v (n=16),
inv(16)(p13q22)/AML M4eo (n=10), and
t(11q23)/MLL-aberrations (n=10)

### Example 6

Microarray experiments. Gene expression analyses were performed from cells remaining from the diagnostic sample. They had immediately been lysed, frozen and were stored at −80° C. from 1 to 34 months until preparation for gene expression profiling. The targets for U95Av2 microarrays were prepared according to current protocols (Affymetrix). Before expression profiling, Test3 Probe Arrays were chosen for monitoring the integrity of the cRNA.

### Example 6

### Results I: Characterization of Leukemia Samples

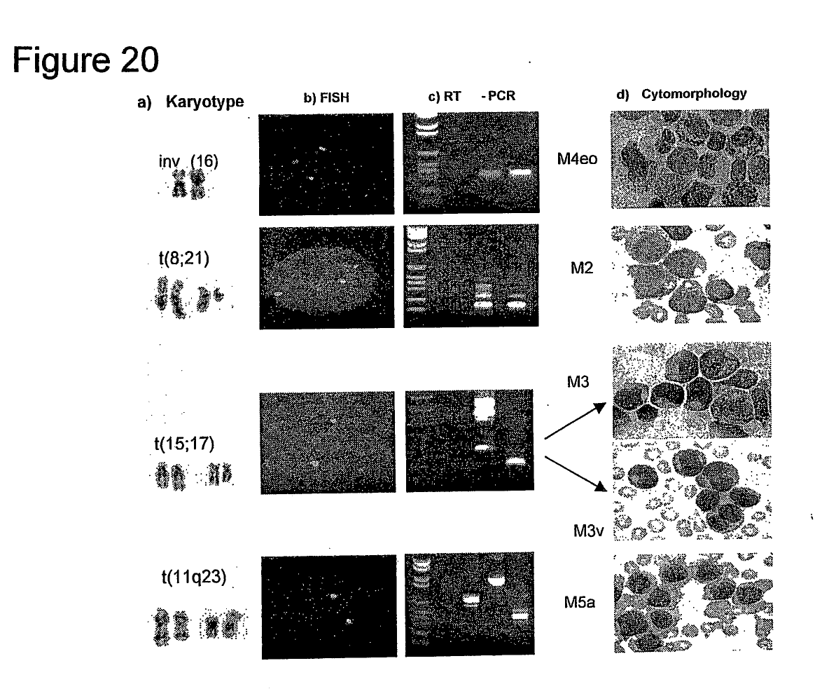

AML samples were thoroughly characterized by a combination of cytomorphology, cytogenetics, FISH, RT-PCR and quantitative real-time PCR (FIG. 20). All patients showed the above mentioned balanced abnormalities as the sole karyotype change. Using FISH analysis, more than 90% of cells demonstrated the specific signal constellation. The respective fusion transcripts AML1-ETO in t(8;21), CBF□-MYH11 in inv(16), PML-RAR□ in t(15;17) and various MLL-fusion partners in t(11q23) were detected by PCR techniques in all samples. These subtypes are specifically associated with five cytomorphological subtypes according to FAB classification: inv(16)(p13q22)/AML M4eo, t(8;21)(q22;q22)/AML M2, t(15;17)(q22;q12)/AML M3/M3v, and t(11q23)/MLL in FAB M5a/b, respectively. AML subtypes M3 and M3v both carry the same chromosome aberration but differ in morphological and clinical aspects.

### Example 6

### Results II: Class Separation

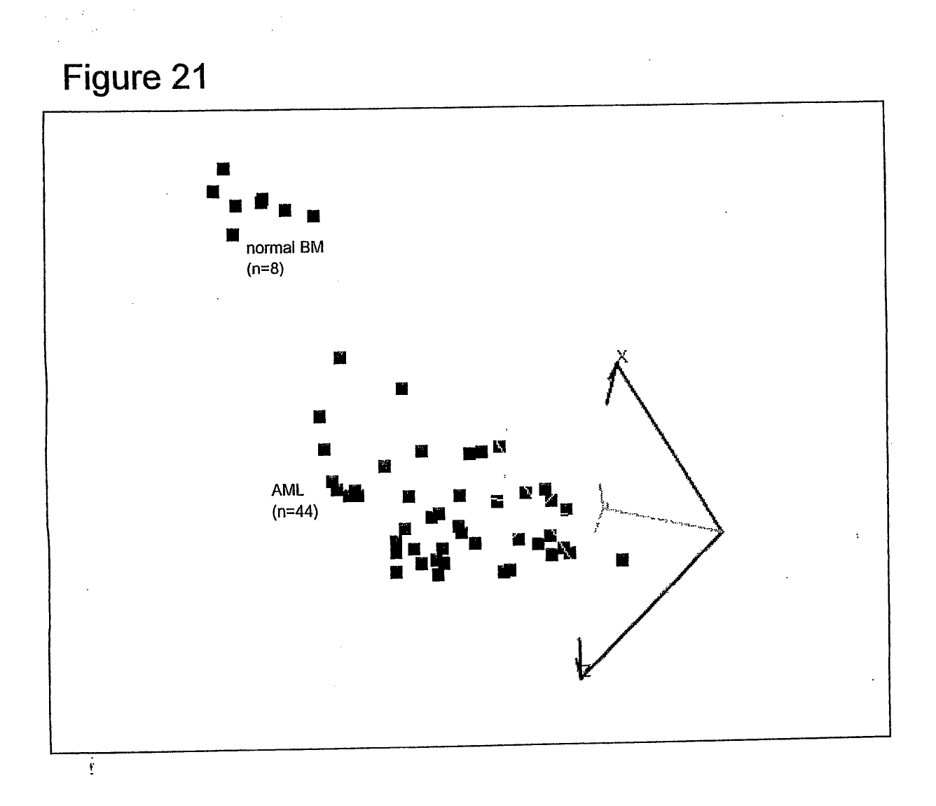

For data analysis we combined different approaches. First, a reduced subset of 200 genes obtained by permutation-based neighborhood analysis (SAM, Tusher et al., 2001) was visualized for corresponding clusters using principal component analysis (J-Express, Dysvik et al., 2001) (FIG. 21). Samples from healthy donors cluster into a distinct group, likewise all AML samples demonstrate homogenity by forming a second cluster.

### Example 6

### Results III: Class Prediction

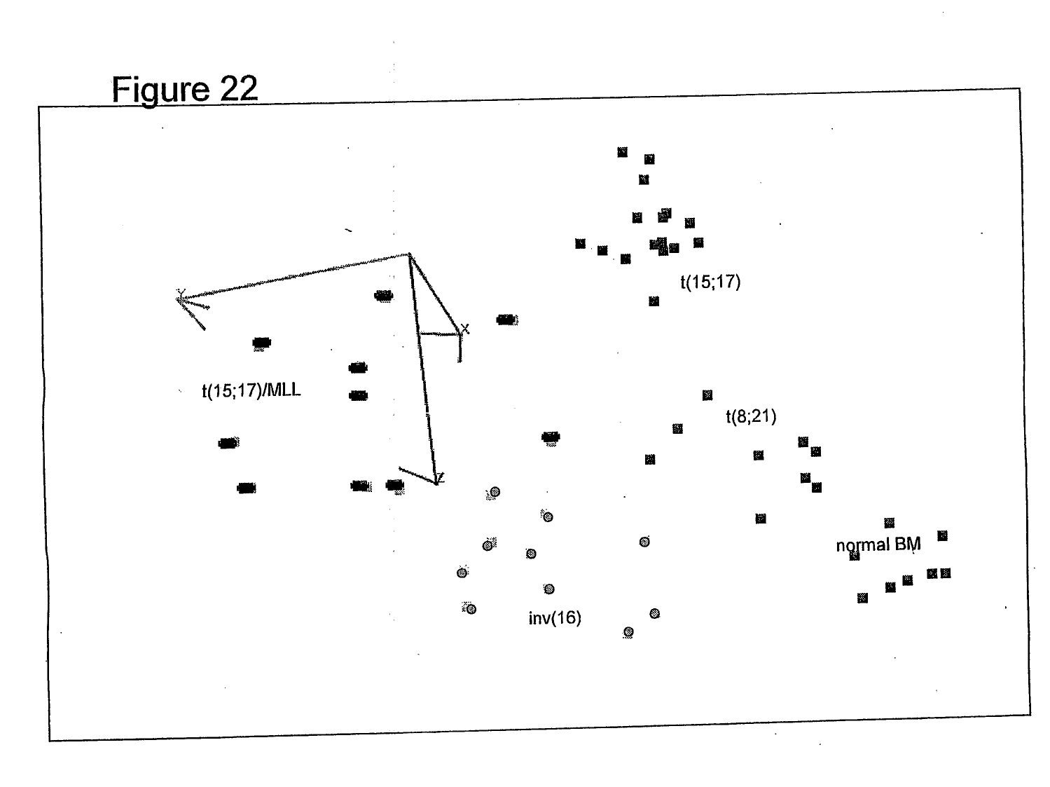

Next, we adapted the signal-to-noise/weighted voting algorithm (Golub et al., 1999) to identify discriminative genes. A minimal set of 39 genes, which provided both optimal classification accuracy and highest prediction strength, was selected to avoid overfitting. The significance of each gene was tested by permutation-based neighborhood analysis. The robustness of the classifier was assessed by leave-one-out crossvalidation. These expression signatures were sufficient to distinguish AML samples with high accuracies from normal bone marrow and to predict the recurrent chromosome aberration, respectively (Table 27, FIG. 22). Table 28a shows for which comparison a gene was important including its statistical significance.

A set of 39 genes is sufficient for class prediction. Accuracy denotes the rate of correctly classified test samples. P(g,c) indicates the signal-to-noise ratio of gene x: S_{x=(μ}₁−μ₂)/(ó₁+ó₂), where μₖ and ók denote the mean expression and standard deviation of gene x in group k. As calculated from pairwise comparisons (class A vs. B), positive P(g,c) values indicate a higher gene expression in class A, negative P(g,c) values a higher gene expression in class B, respectively. HGNC symbols are given in column 1.

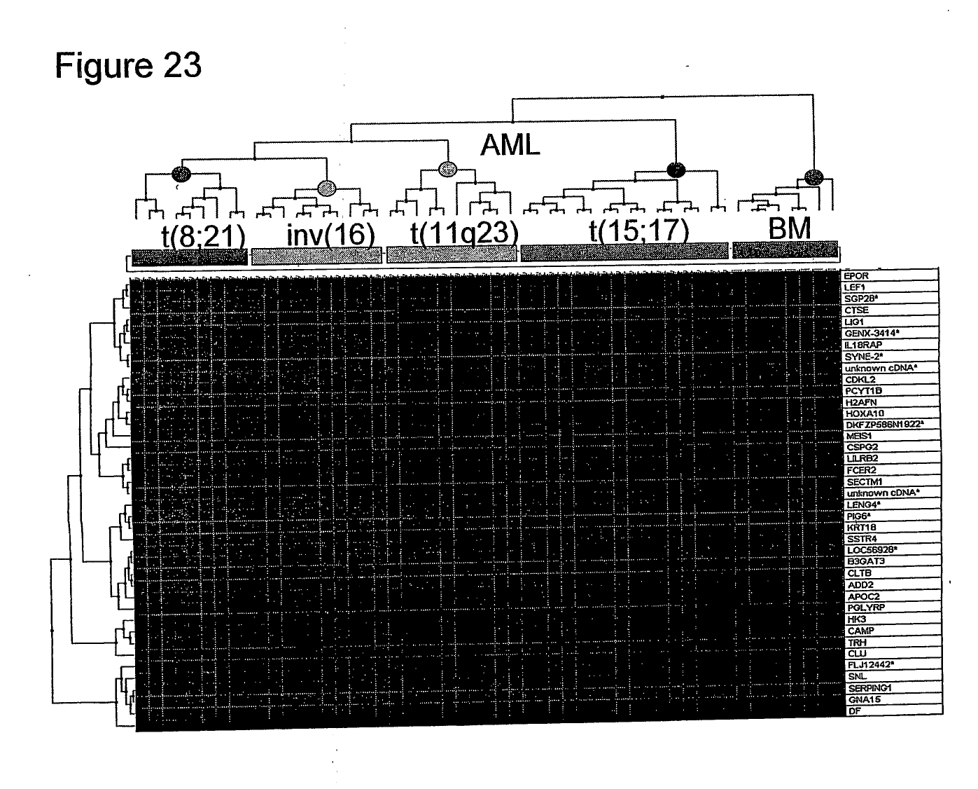

All leukemia samples could accurately be assigned to their corresponding cytogenetic subtype with 100% accuracies. To illustrate these results, a hierarchical clustering is shown (FIG. 23).

### Example 6

### Conclusions

The expression pattern of 39 genes allowed precise class assignments of four cytogenetically defined AML subtypes according to the WHO classification of hematological malignancies, and normal BM, respectively.
Thus, we introduce candidate genes suitable for diagnosis of AML subgroups based on gene expression profiling.
Potentially, gene expression patterns will allow the additional subclassification of AML, especially in subtypes with no specific cytogenetic markers (e.g. normal karyotype).

### Example 7

### Gene Expression Profiles of Distinct Leukemia Types and Subtypes: A Study of 280 Patients Using High-Density Microarrays

### Example 7

### Introduction

Here, we demonstrate gene expression analyses of 9 healthy BM donors and 271 leukemia patients representing:

AML: 4 distinct cytogenetic subtypes t(8;21)(q22;q22) (AML t(8;21)), inv(16)(p13q22) (AML inv(16)), t(15;17)(q22;q12) (AML t(15;17)), and t(1 q23)/MLL (AML MLL). In addition, we analyzed AML samples characterized by normal karyotypes (AML normal), complex aberrant karyotypes (AML complex), trisomy 8 as sole aberration (AML+8), and other chromosomal changes (AML other).

ALL: 3 distinct genetically defined subtypes: t(4;11)(q21;q23) (ALL t(4;11)), t(8;14)(q24;q32) (ALL t(8;14)), t(9;22)(q34;q11) (ALL Ph) and 2 subtypes defined by their immunophenotype: ALL of the B-lineage not carrying the t(9;22) (ALL B not Ph) and T-ALL (T-ALL)

CLL: 5 genetically defined subtypes: trisomy 12 (tri 12), deletion 11q (11q-), deletion 13q (13q-), deletion 17p (17p-) and none of these aberrations (normal)

CML (CML) without any further subdivision and

Normal bone marrow from healthy volunteers (normal BM).

We used the Affymetrix oligonucleotide microarray technology (GeneChip® Instrument System) to obtain gene expression profiles of each individual clinical sample of interest. The commercially available HG-U133 probe arrays gave information about the relative mRNA abundance of about 33,000 human genes which are represented on these high-density DNA-oligonucleotide microarrays.

Chip Information (as provided by manufacturer):

The GeneChip® Human Genome U133 Set (HG-U133A and HG-U133B) is comprised of two microarrays containing over 1,000,000 unique oligonucleotide features covering more than 39,000 transcript variants, which in turn represent greater than 33,000 of the best characterized human genes. This powerful set allows to reproducibly examine the quantitative and qualitative expression of most genes in the human genome, and was designed using the recently published and publicly available draft of the human genome sequence. Sequences used in the design of the array were selected from GenBank, dbEST, and RefSeq. Sequence clusters were created from Build 133 of UniGene (Apr. 20, 2001) and refined by analysis and comparison with a number of other publicly available databases including the Washington University EST trace repository and the University of California, Santa Cruz golden-path human genome database (April 2001 release). In addition, ESTs were analyzed for untrimmed low-quality sequence information, correct orientation, false priming, false clustering, alternative splicing and alternative polyadenylation.

Combining different approaches for data analysis, a set of genes was identified to designate a reliable class prediction model. Based on the expression pattern of those genes, defined leukemia types and subtypes could accurately be predicted and separated from healthy BM. Taken together, gene expression signatures demonstrate a very close correlation between genotype and gene expression.

Therefore, introducing a set of candidate genes, measurements of mRNA abundancies by gene expression profiling serves for diagnosis of leukemia types and subtypes.

### Example 7

### Material and Methods

We analyzed BM aspirates from 9 healthy volunteers and the following 280 leukemia patients:

Acute myeloid leukemia (AML)

t(8;21)(q22;q22)/AML M2 (n=13),

t(15;17)(q22;q12)/AML M3/M3v (n=20),

inv(16)(p13q22)/AML M4eo (n=12),

t(11q23)/MLL-aberrations (n=15)

trisomy 8 (n=10)

normal karyotype (n=62)

complex aberrant karyotype (n=36)

other aberrations (n=5)

Acute lymphoblastic leukemia (ALL)

t(4;11)(q21;q23) (n=9)

t(8;14)(q24;q32) (n=4)

t(9;22)(q34;q11) (ALL Ph) (n=15)

ALL B lineage without t(9;22) (ALL B not Ph) (n=9)

T-ALL (n=9) Chronic lymphocytic leukemia (CLL)

trisomy 12 (tri 12) (n=5)

deletion 11q (11q-) (n=4)

deletion 13q (13q-) (n=10)

deletion 17p (17p-) (n=4)

none of these aberrations (normal) (n=9)

Chronic myeloid leukemia (n=14)

Normal bone marrow (normal BM) (n=9)

### Example 7

### Results I: Characterization of Leukemia Samples

We selected bone marrow (BM) samples from 271 leukemia patients at diagnosis representing 18 different disease entities or subentities and from 9 healthy volunteers, respectively. All cases were sent for reference diagnostics to our laboratory, registered in our leukemia database and were treated within prospective randomized multi-center trials. The studies abide by the rules of the local internal review board and the tenets of the revised Helsinki protocol. Samples were received either locally or by overnight mail. Diagnosis was performed by an individual combination of cytomorphology, cytogenetics, FISH, immunophenotyping and molecular genetics. Mononuclear cells were isolated by a Ficoll gradient, lysed, frozen and were stored at −80° C. from one to 34 months until sample preparation for gene expression analysis. All leukemia samples were thoroughly characterized by a individual combination of cytomorphology, cytogenetics, immunophenotyping, fluorescence in situ hybridisation (FISH), polymerase chain reaction based methods both qualitative RT-PCR and quantitative real-time PCR. Using FISH analysis, more than 90% of cells demonstrated the specific signal constellation. The respective fusion transcripts BCR-ABL in t(9;22) positive CML (Schoch et al. 2002a) and in t(9;22) positive ALL, AML1-ETO in AML with t(8;21), CBFbeta-MYH11 in AML with inv(16), PML-RARalpha in AML with t(15;17) (Schoch et al. 2002b) and various MLL-fusion partners in both AML and ALL with t(11q23) were detected by FISH and PCR techniques in all samples.

In t(8;14) positive ALL the IGH-C-MYC rearrangement was confirmed by FISH. In all cases with AML and complex aberrant karyotype 24 color FISH was performed in addition to chromosome banding analysis (Schoch et al. 2002c).

Genetic subtyping of CLL was carried out using interphase FISH with the following probes (Buhmann et al. 2002):
for the detection of trisomy 12 a centromere specific probe for chromosome 12
for the detection of 11q deletions probes for the ATM as well as for the RDX gene
for the detection of 13q deletions probes for the retinoblastoma gene (Rb), and the anonymous loci D13S25 and D13S319
for the detection of 17p deletion a probe for the p53 gene
cases with none of the above mentioned aberrations were assigned to the group normal

### REFERENCES

Buhmann R, Kurzeder C, Rehklau J. Westhaus D, Bursch S, Hiddemann W, Haferlach T. Hallek M, Schoch C.
CD40L stimulation enhances the ability of conventional metaphase cytogenetics to detect chromosome aberrations in B-cell chronic lymphocytic leukaemia cells.
Br J Haematol 2002 September; 118(4):968-75
Schoch C, Schnittger S, Kern W, Lengfelder E, Loffler H, Hiddemann W, Haferlach T.
Rapid diagnostic approach to PML-RARalpha-positive acute promyelocytic leukemia.
Hematol J 2002a; 3(5):259-63
Schoch C, Schnittger S, Bursch S, Gerstner D, Hochhaus A, Berger U, Hehlmann R, Hiddemann W, Haferlach T.
Comparison of chromosome banding analysis, interphase- and hypermetaphase-FISH, qualitative and quantitative PCR for diagnosis and for follow-up in chronic myeloid leukemia: a study on 350 cases, Leukemia 2002b Jan; 16(1):53-9
Schoch C, Haferlach T. Bursch S, Gerstner D, Schnittger S, Dugas M, Kern W, Löffler H, Hiddemann W.
Loss of genetic material is more common than gain in acute myeloid leukemia with complex aberrant karyotype: A detailed analysis of 125 cases using conventional chromosome analysis and fluorescence in situ hybridization including 24-color FISH.
Genes Chromosomes Cancer 2002 September; 35(1):20-9

### Example 7

### Results II: Sample Preparation and Microarray Hybridisation

Microarray analyses were performed utilising the GeneChip® System (Affymetrix, Santa Clara, USA). The targets for GeneChip® analyses were prepared according to the current Expression Analysis Technical Manual. Briefly, lysates of the leukemia samples were homogenised (QIAshredder, Qiagen, Hilden, Germany) and total RNA extracted (RNeasy Mini Kit, Qiagen). Normally, 5 μg total RNA isolated from 1×10⁷ cells were used as starting material in the subsequent cDNA-synthesis using oligo[(dT)₂₄T7promotor]₆₅ primer (cDNA Synthesis System, Roche Applied Science, Mannheim, Germany). The cDNA was purified by phenol:chloroform:isoamyl alcohol (25:24:1) extraction (Ambion, Austin, USA) and acetate/ethanol precipitated over night. For detection of the hybridised target nucleic acid biotin-labeled ribonucleotides were incorporated during the in vitro transcription (Enzo® BioArray™ HighYield™ RNA Transcript Labeling Kit, ENZO, Farmingdale, USA). After quantification of the purified cRNA (RNeasy Mini Kit, Qiagen), 15 μg labeled cRNA were fragmented by alkaline treatment (200 mM Tris-acetate, pH 8.2, 500 mM potassium acetate, 150 mM magnesium acetate) and added to the hybridisation cocktail sufficient for 5 hybridisations on standard format GeneChip® microarrays. Before hybridisation to HG-U133 microarrays, Test3 microarrays (Affymetrix) were chosen in some cases for monitoring the integrity of the cRNA. Washing and staining of the probe arrays was performed according to the current protocols of the manufacturer (Fluidics Station, Micro_{—}1v1, EukGE-WS2v4). The Affymetrix software (Microarray Suite, Version 5.0) extracted fluorescence intensities from each feature on the microarrays as detected by confocal laser scanning according to the manufacturers recommendations. Some of the hybridization cocktails had previously been hybridized to U95Av2 arrays. Hybridization cocktails can be used for up to 5 distinct array analyses.

All hybridisation cocktails demonstrated high quality cRNA characteristics. We considered both low 3′/5′ ratio (e.g., lower than about 3) of housekeeping controls and the total number of present called genes (> about 30% on U133A), along with the average signal intensity of a present called gene. Expression profiles which fulfilled all quality control criteria were selected for subsequent supervised selection of informative genes.

### Example 7

### Results III: Statistical Analyses

For data analysis we combined different approaches. First, the expression data was preprocessed. Raw expression intensities were scaled using the Affymetrix Microarray Suite software scaling parameter (target intensity: 5000). This preprocessing is based on a mask file which compares expression intensities of a set of 100 genes which code for ubiquitous housekeeping cellular proteins. This set of genes for normalisation of expression intensities is represented on both U133A and U133B arrays. The step of data preprocessing assures that array experiments can be compared properly using further statistical algorithms and methods. Subsequently, the data was analyzed according to two different established methods from as described below. The results from the two analyses were systematically compared to validate the list of differentially expressed genes.

1. Selection of Differentially Expressed Genes

a) Analysis According to Example 3.

The top 20 differentially expressed genes were calculated for all disease entities and normal bone marrow, respectively, as described in example 3. Expression data were analyzed in order to select a minimal set of discriminative genes, which provides, as described hereinabove (Example 3), maximum classification accuracy in leave-one-out-crossvalidation.

One-versus-all (OVA) and all-pairs comparisons (AP) were systematically applied. Genes were ranked according to signal-to-noise ratio (STN). For each OVA and AP comparison a set of discriminative genes is disclosed in tables 29, 32, 35, 38 and 41 whereby the gene names can be found in tables 43a,b. The most discriminative and informative genes are marked by asterisks in tables 29, 32, 35, 38 and 41. Classification accuracy was estimated by means of leave-one-out-crossvalidation and weighted voting.

### REFERENCES

Golub T R, Slonim D K, Tamayo P, Huard C, Gaasenbeek M, Mesirov J P, Coller H, Loh M L, Downing J R, Caligiuri M A, Bloomfield C D, Lander E S. Molecular classification of cancer: class discovery and class prediction by gene expression monitoring. Science 1999; 286(5439):531-7
Pomeroy S L, Tamayo P, Gaasenbeek M, Sturla L M, Angelo M, McLaughlin M E, Kim J Y, Goumnerova L C, Black P M, Lau C, Allen J C, Zagzag D, Olson J M, Curran T, Wetmore C, Biegel J A, Poggio T, Mukherjee S, Rifkin R, Califano A, Stolovitzky G, Louis D N, Mesirov J P, Lander E S, Golub T R. Prediction of central nervous system embryonal tumour outcome based on geneexpression. Nature 2002; 415(6870):436-42.
   2. Estimation of Classification Accuracy

A set of 20 top-ranked genes, which provided both optimal classification accuracy and highest prediction strength, was selected to avoid overfitting. The significance of each gene was tested by permutation-based neighborhood analysis. The robustness of the classifier was assessed by leave-one-out crossvalidation. These expression signatures were sufficient to distinguish leukemia samples with high accuracies from normal bone marrow and also to predict the recurrent chromosome aberration, respectively (Tables 29, 32, 35, 38, 41). Accuracy denotes the rate of correctly classified test samples. P(g,c) indicates the signal-to-noise ratio of gene x: Sₓ=(μ₁−μ₂)/( ó₁+ó₂), where μₖ and óₖ denote the mean expression and standard deviation of gene x in group k. As calculated from pairwise comparisons (class A vs. B), positive P(g,c) values indicate a higher gene expression in class A, negative P(g,c) values a higher gene expression in class B, respectively.

B) Analysis According to Westfall & Young the Same Data Set was Analysed According to Westfall & Young to Identify Significantly Differentially Expressed Genes with Adjustment of the P-Values for Multiple Testing.

Step-down maxT and minP multiple testing procedures were applied, which compute permutation adjusted p-values for the step-down maxT and minP multiple testing procedures, which provide strong control of the family-wise Type I error rate (FWER). The multitest package (version 1.0) from Bioconductor was applied, which is based on the R statistical language. These methods outperform other methods (see Dudoit, JASA 2002).

### REFERENCES

Westfall P H, Young S S (1993) Resampling-based multiple testing: Examples and methods for p-value adjustment. John Wiley & Sons. ISBN 0-471-55761-7
Dudoit S, Fridlyand J, Speed T P.
Comparison of Discrimination Methods for the Classification of Tumors Using Gene Expression Data. JASA 2002; 97:77-87

Package multtest (version 1.0)

from Bioconductor http://www.bioconductor.org

R statistical language: http://www.r-project.org/

C) Comparison of Gene Lists

The list of differentially expressed genes obtained from 1a) and 1b) were systematically compared using PERL scripts in order to identify genes that occurred in both list, versus genes occurring in one list only.

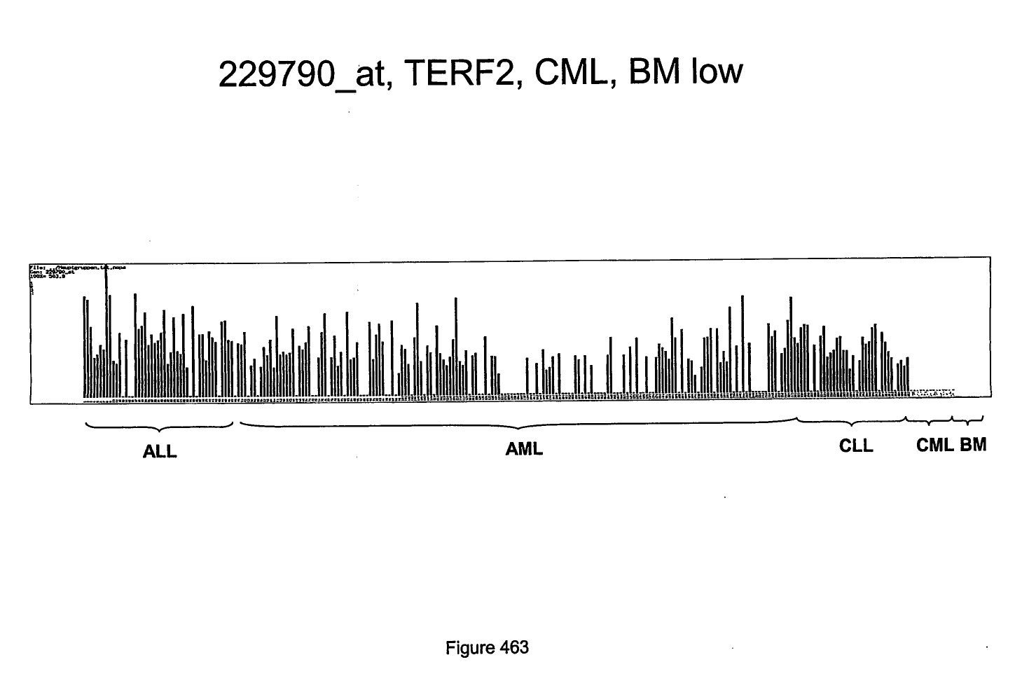

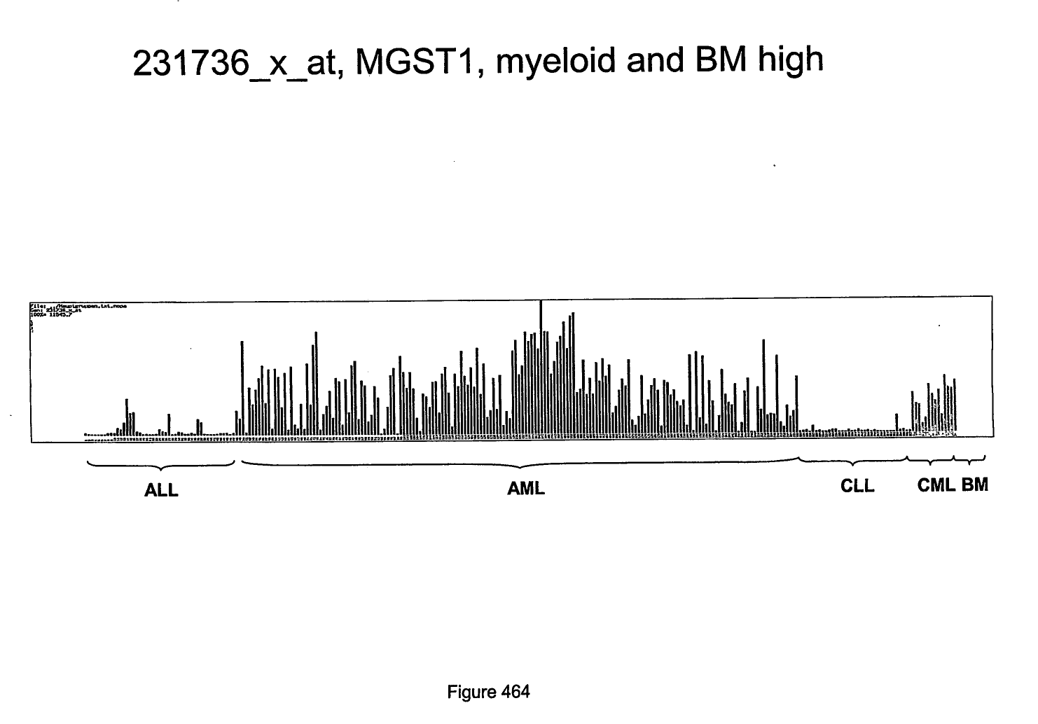

Expression intensities (expression levels) derived from the above-mentioned MicroArray Suite program were plotted as bar graphs showing gene expression profiles using a Per script (FIGS. 24 to 464).

### REFERENCES

PERL: http://www.perl.com

Sensitivities for the detection of leukemia types and subtypes were calculated as the number of positive samples predicted divided by the number of true positives.

Specificities for the detection of leukemia types and subtypes were calculated as the number of negative samples predicted divided by the number of true negatives.

### Example 7

### Results IV: Analysis of 14 Leukemia Subtypes and Normal Bone Marrow

Here we analyzed in total 14 distinct leukemia types and subtypes as well a cohort of healthy volunteers for normal bone marrow characteristics. We applied the described two different statistical methods for identification of genes which allow accurate class assignments to the respective groups.

ALL t(4; 11) (n=9)

ALL t(8;14) (n=4)

ALL B not Ph (n=9)

ALL Ph (n=15)

T-ALL (n=9)

AML+8 (n=10)

AML complex (n=36)

AML normal (n=62)

AML t(8;21) (n=13)

AML(15;17) (n=20)

AML inv(16) (n=12)

AML MLL (n=15)

CLL (n=32)

CML (n=14)

normal BM (n=9)

total: 269 samples

First, expression data were analyzed according to example 3, as described hereinabove.

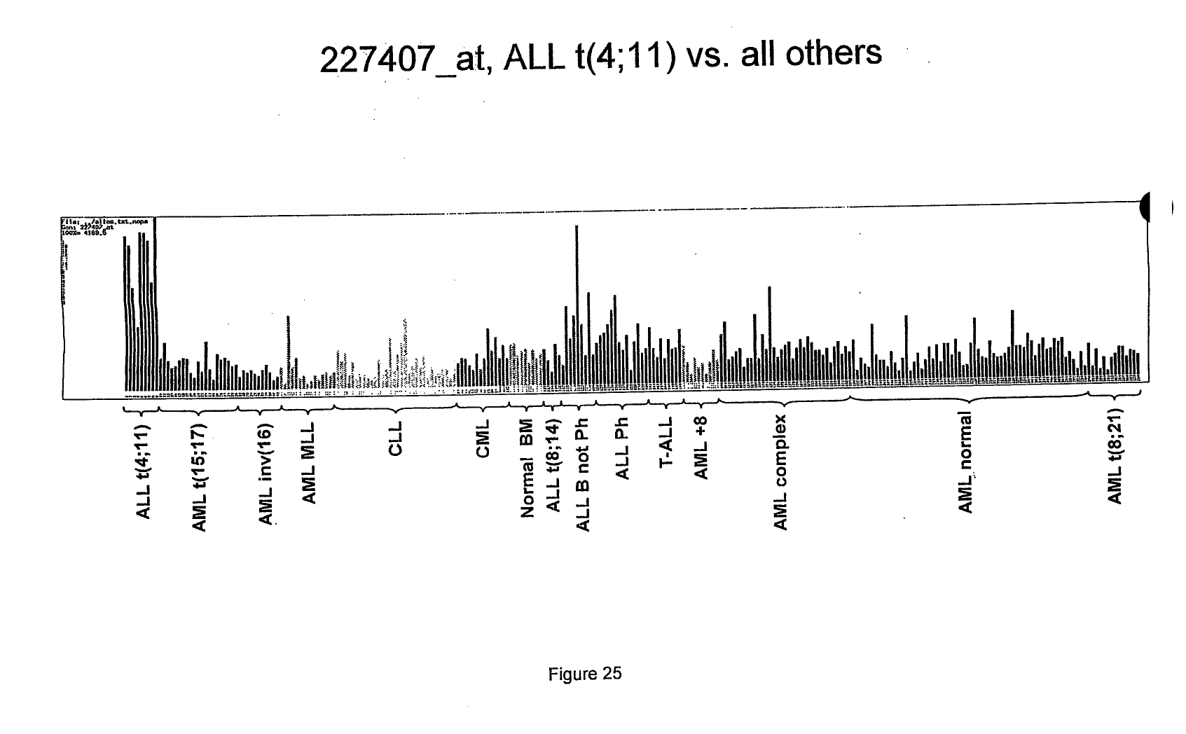

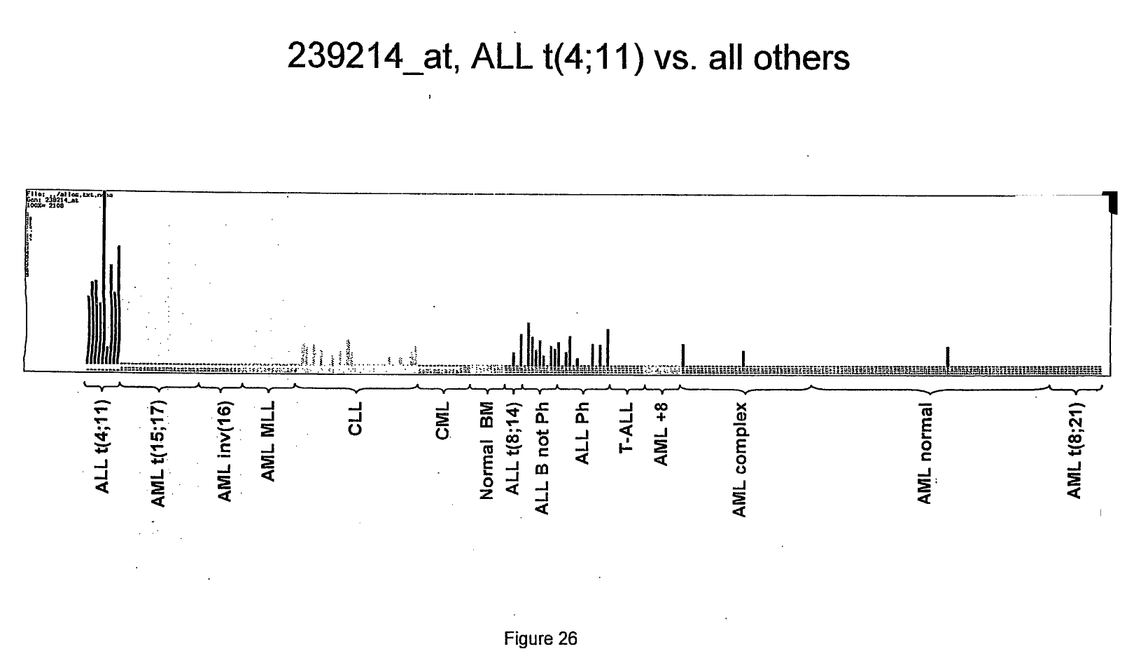

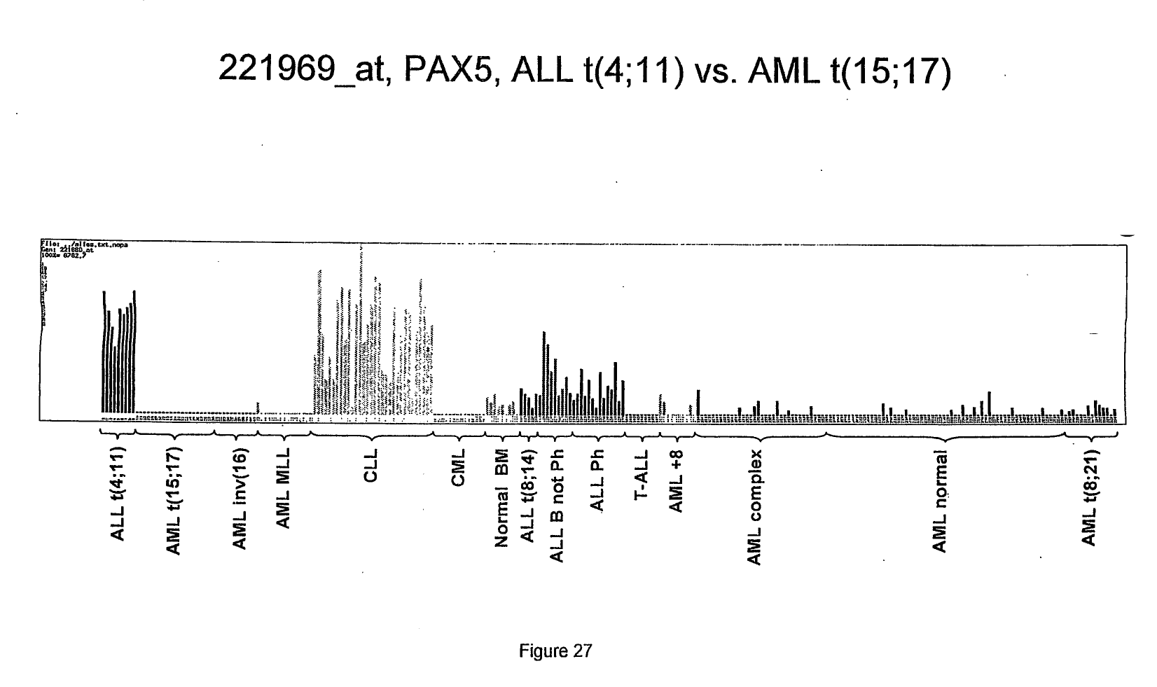

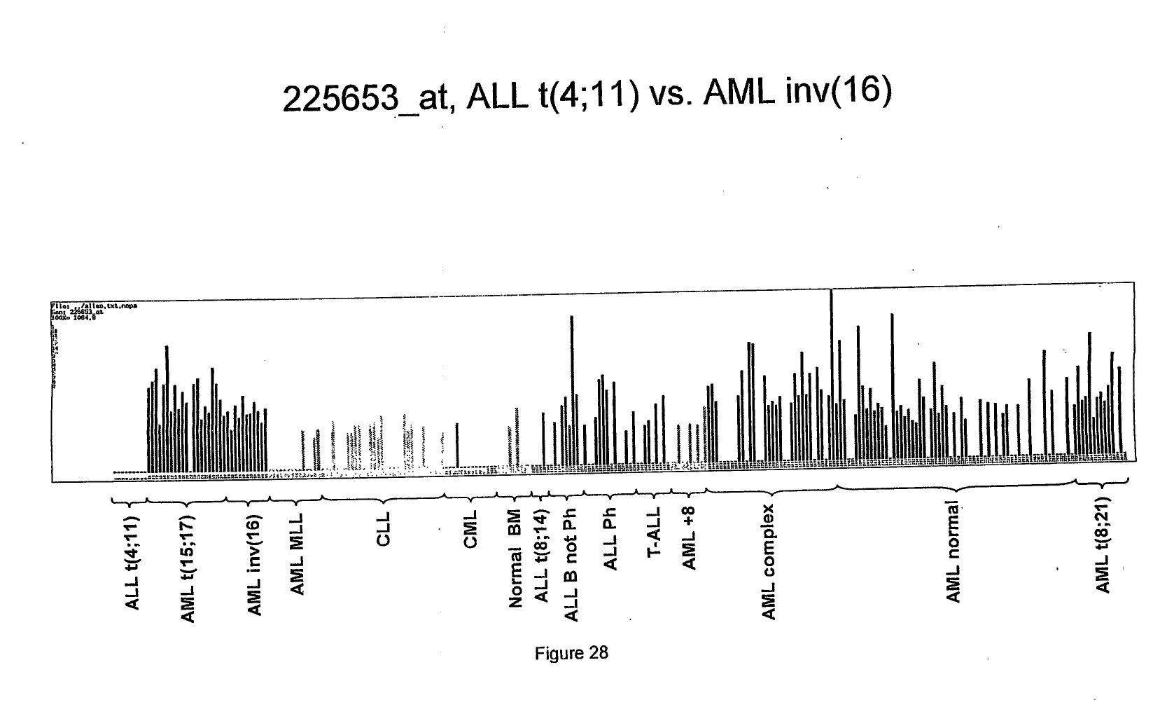

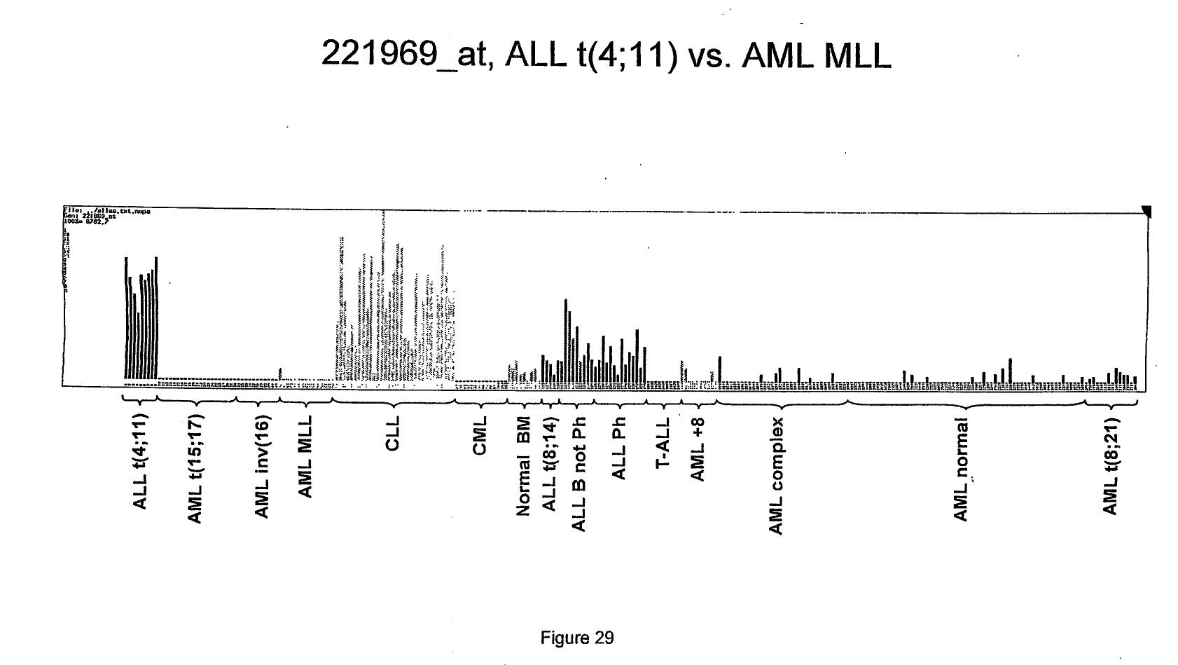

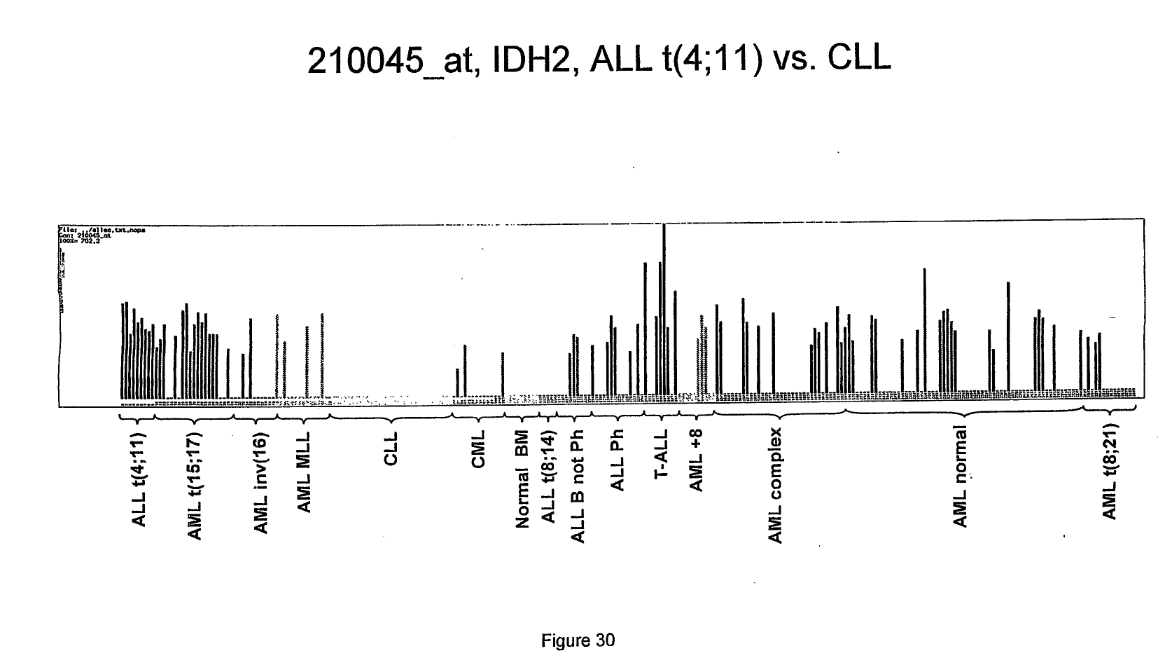

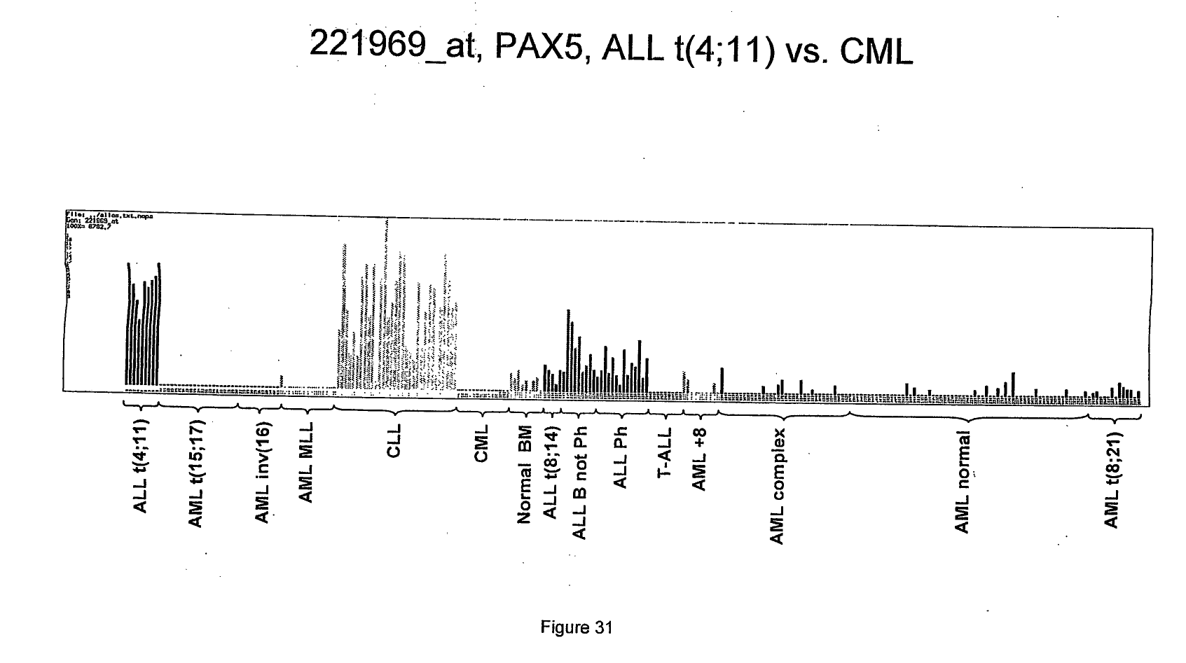

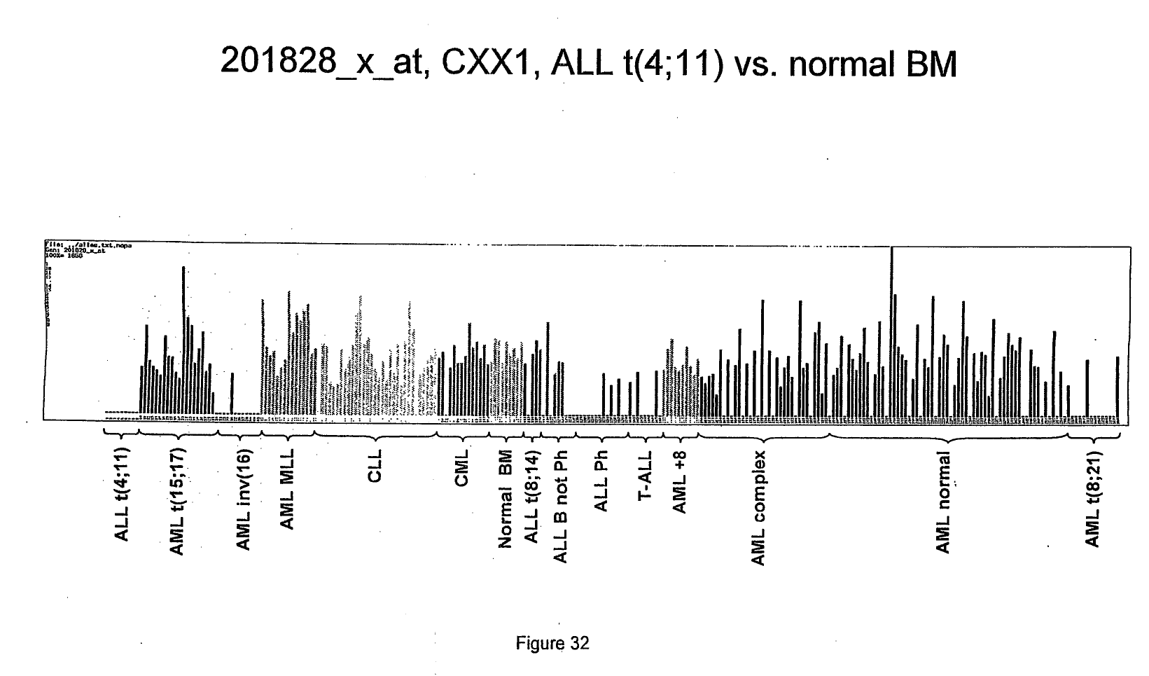

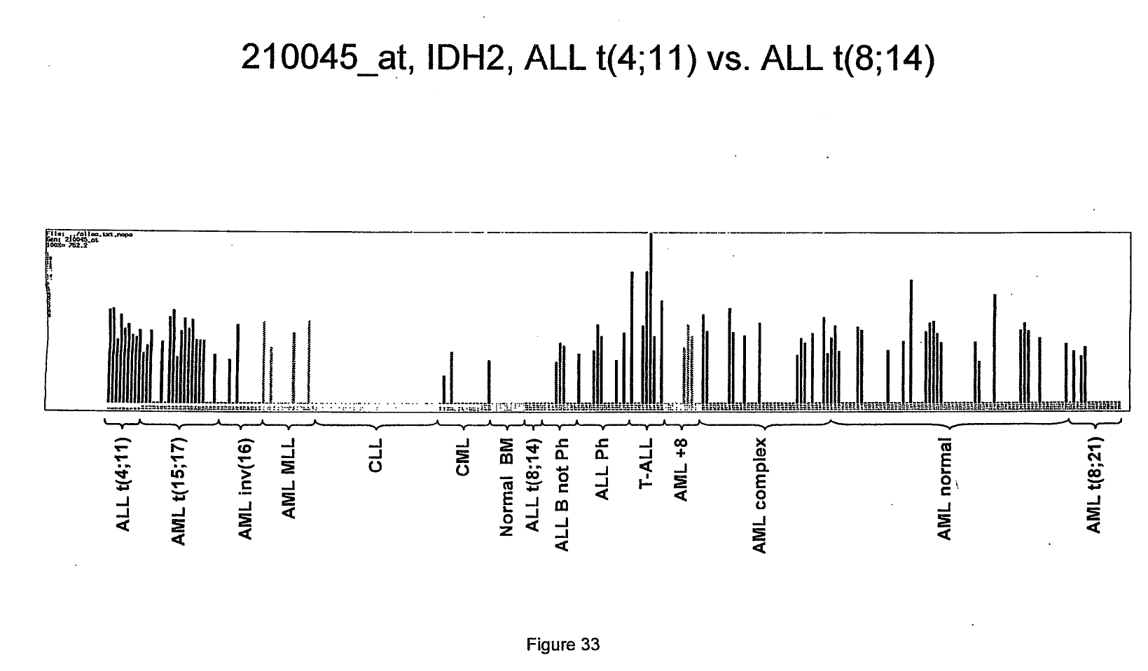

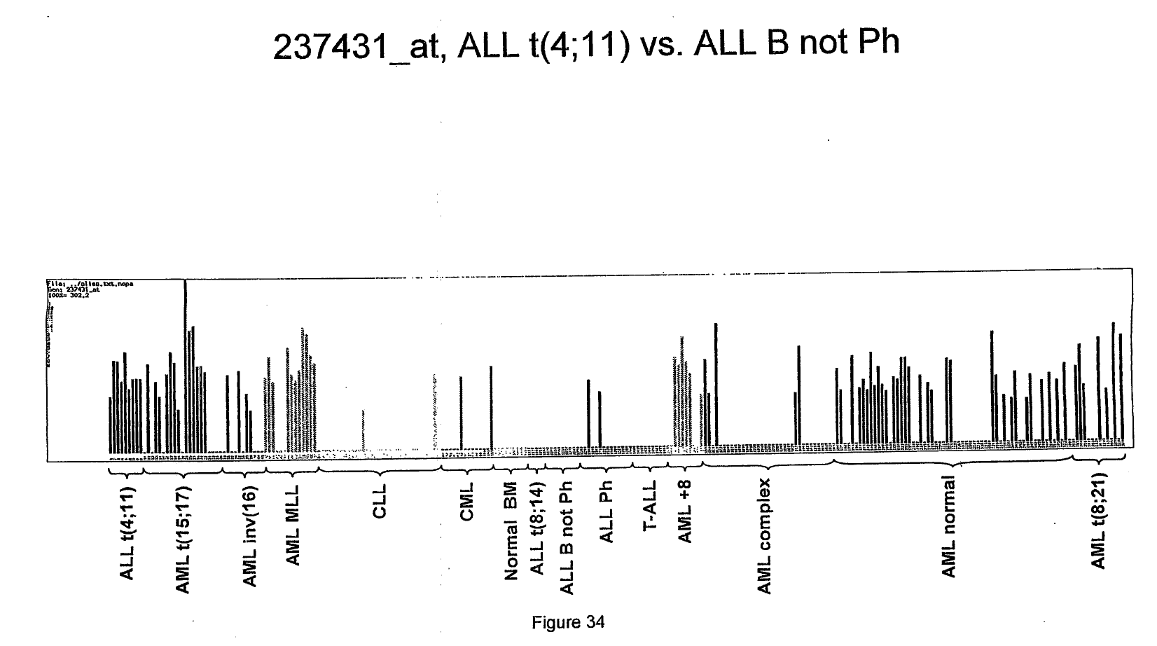

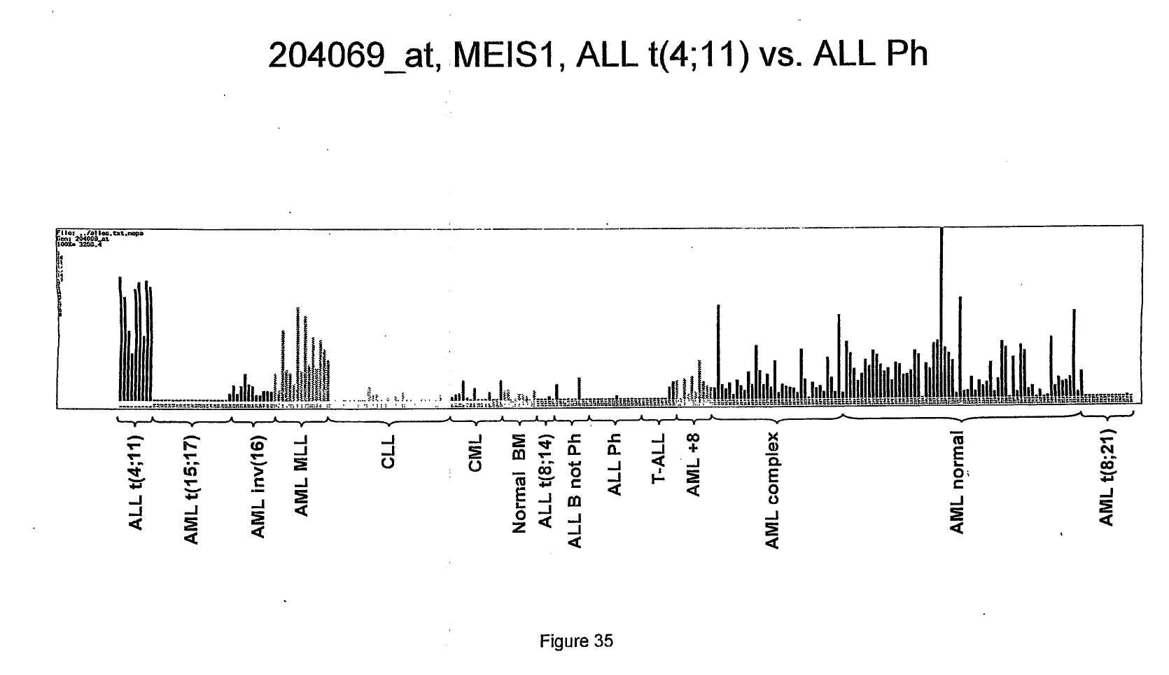

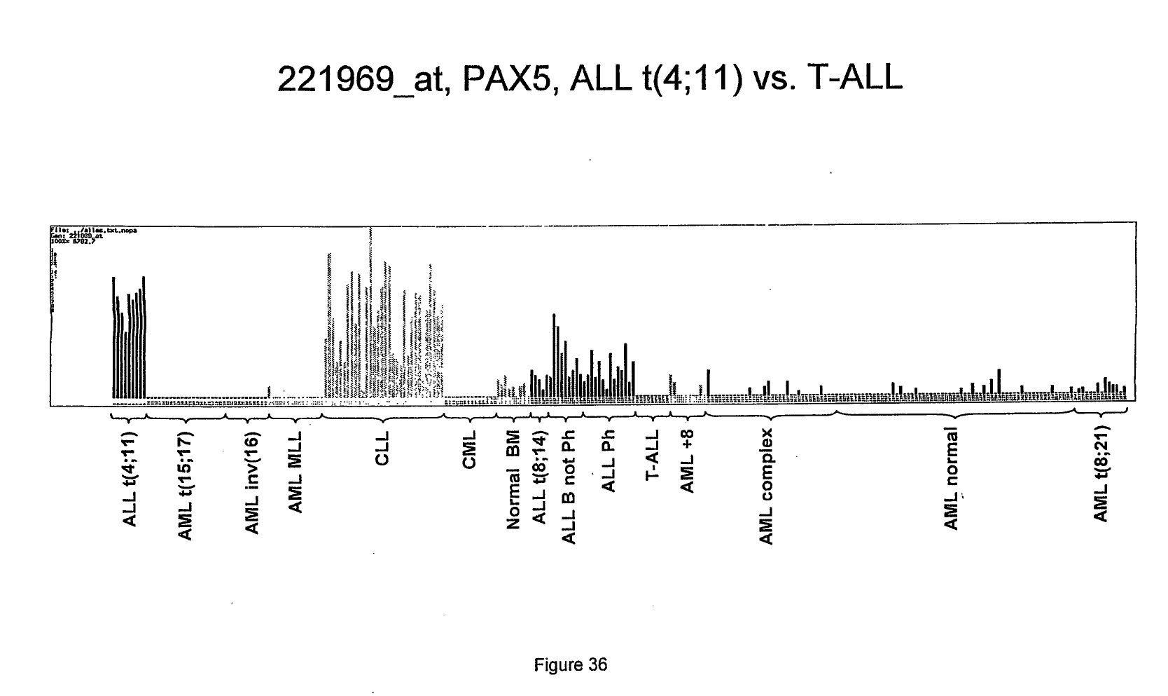

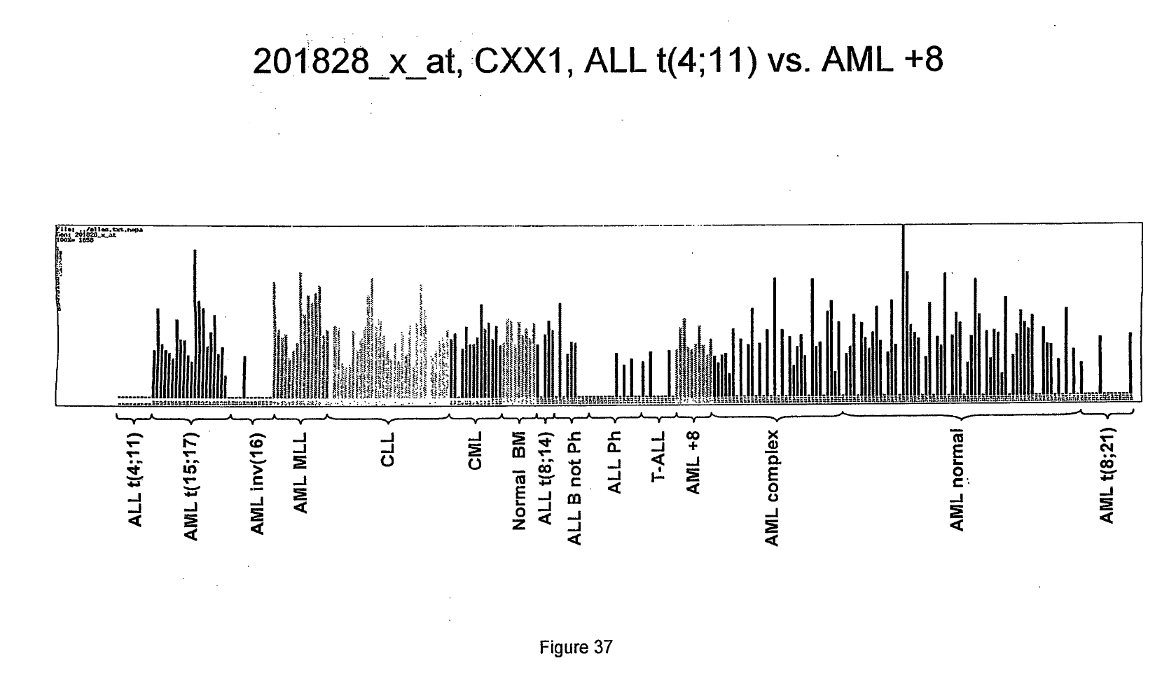

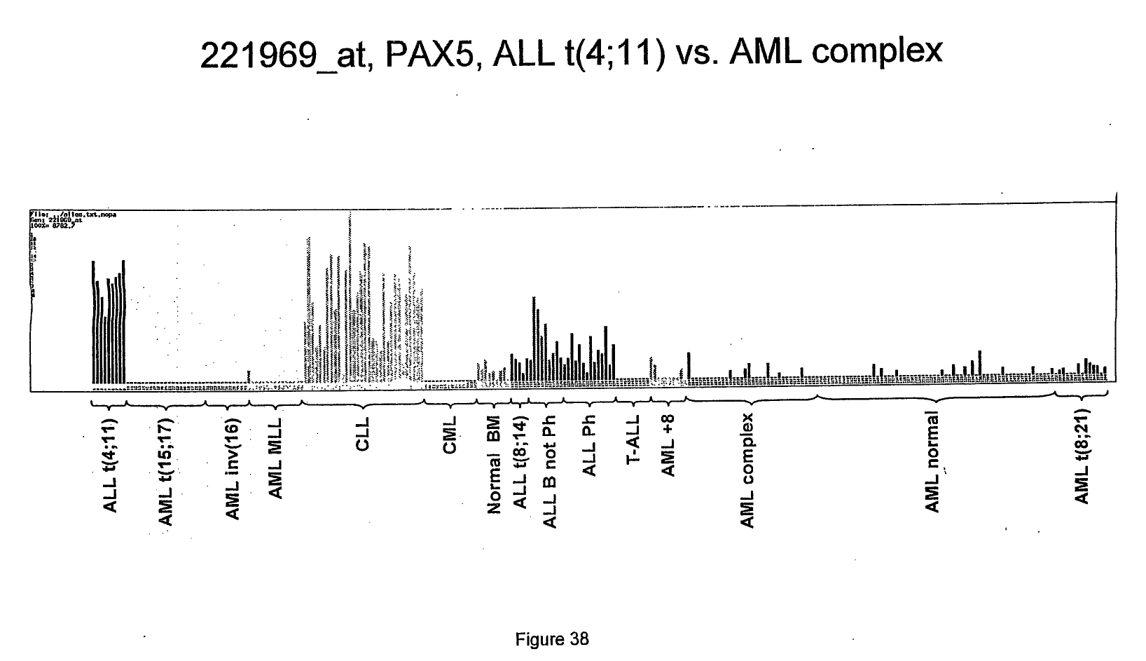

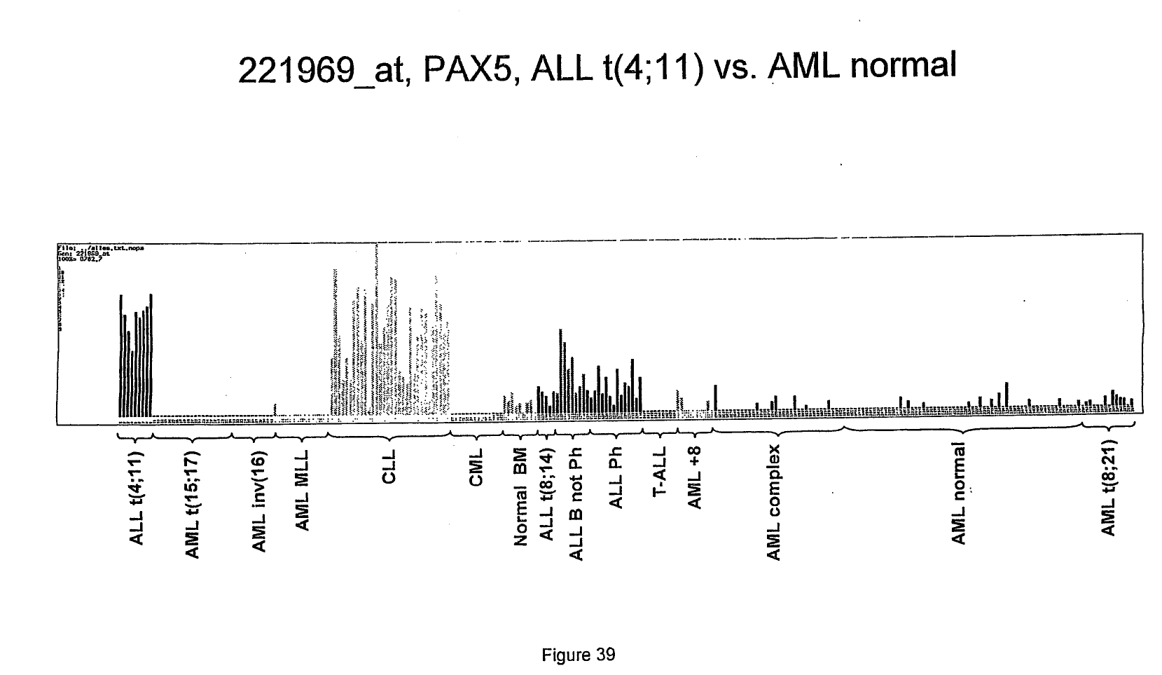

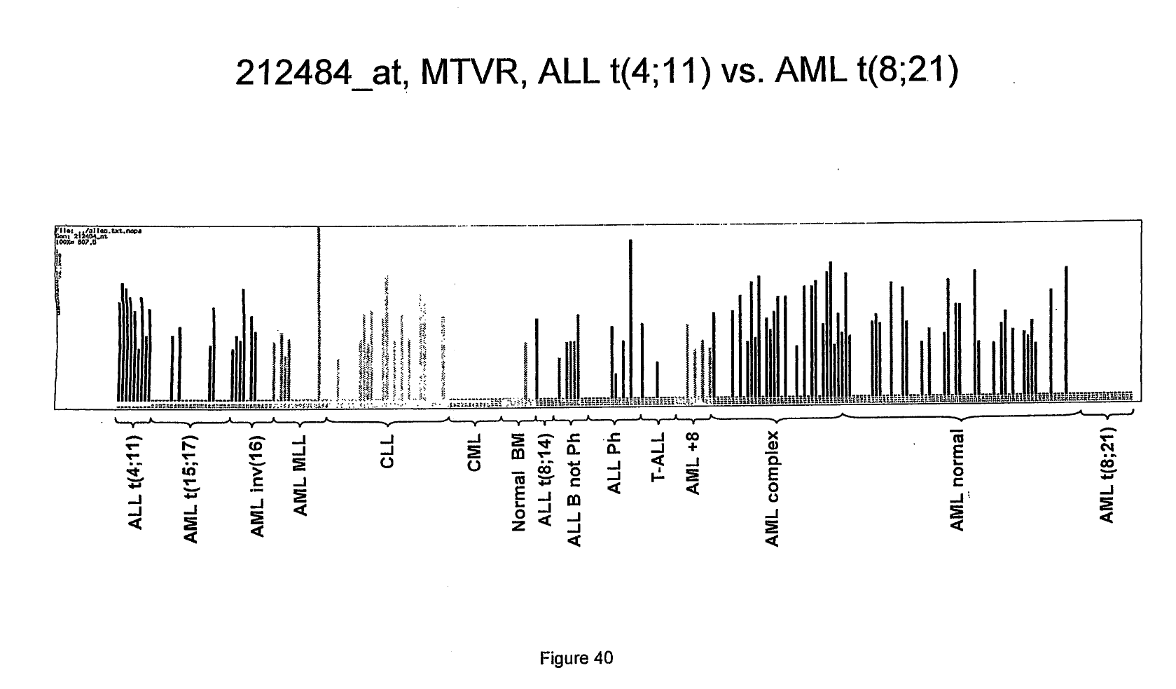

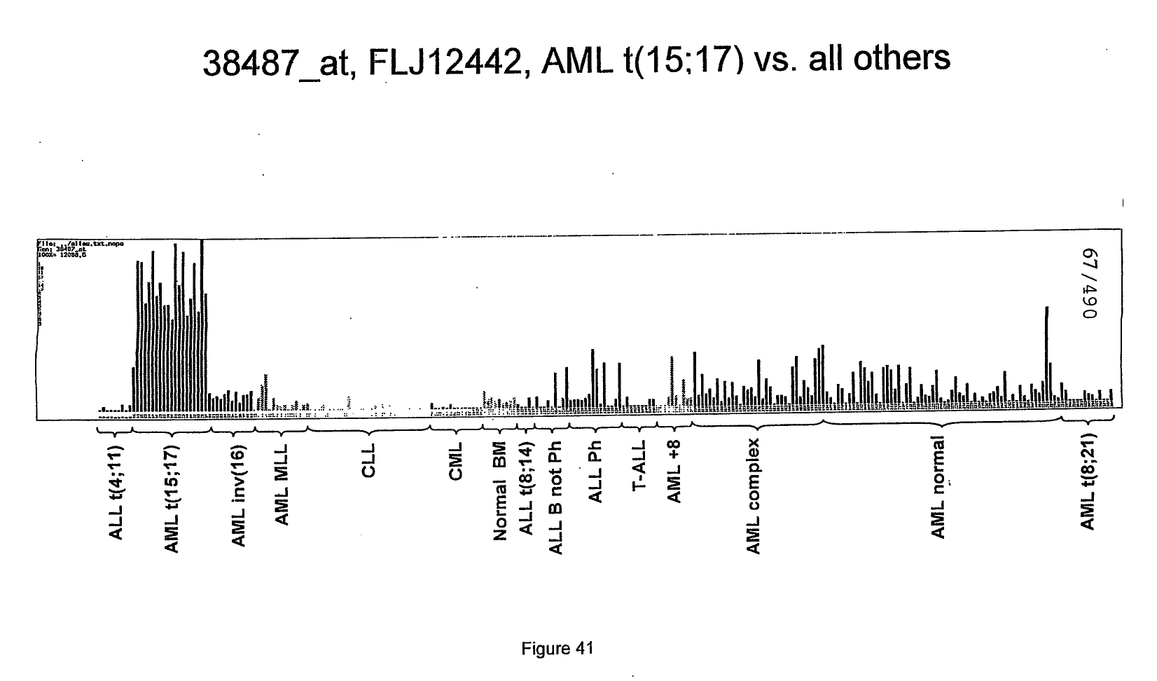

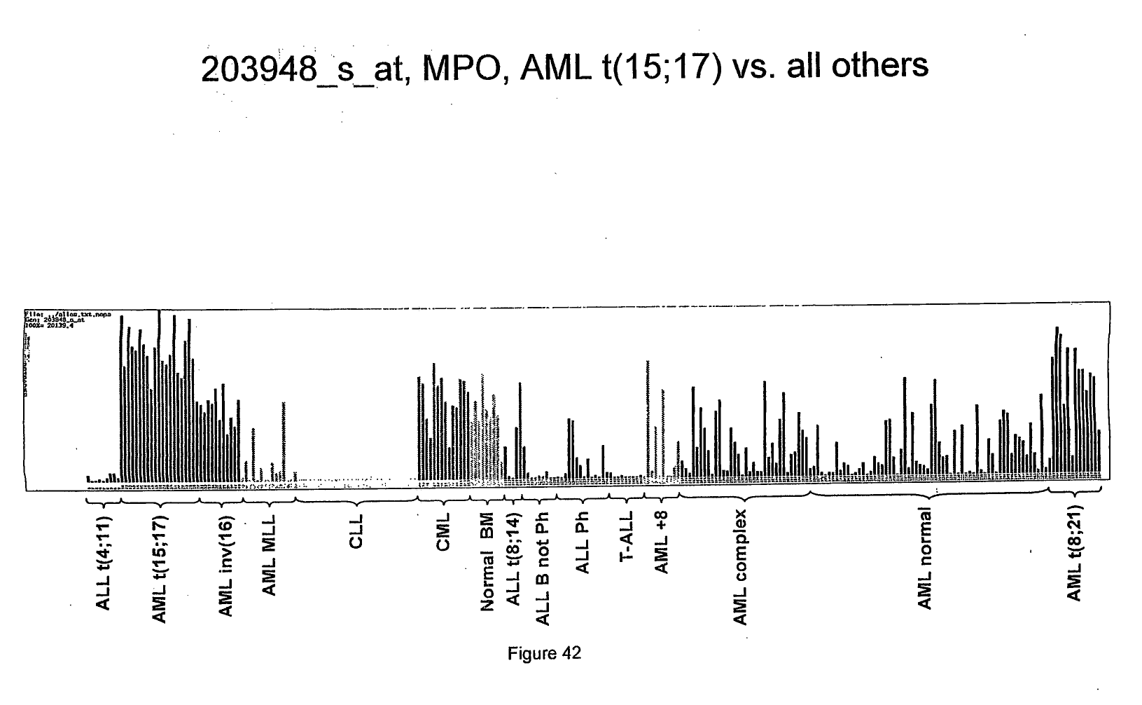

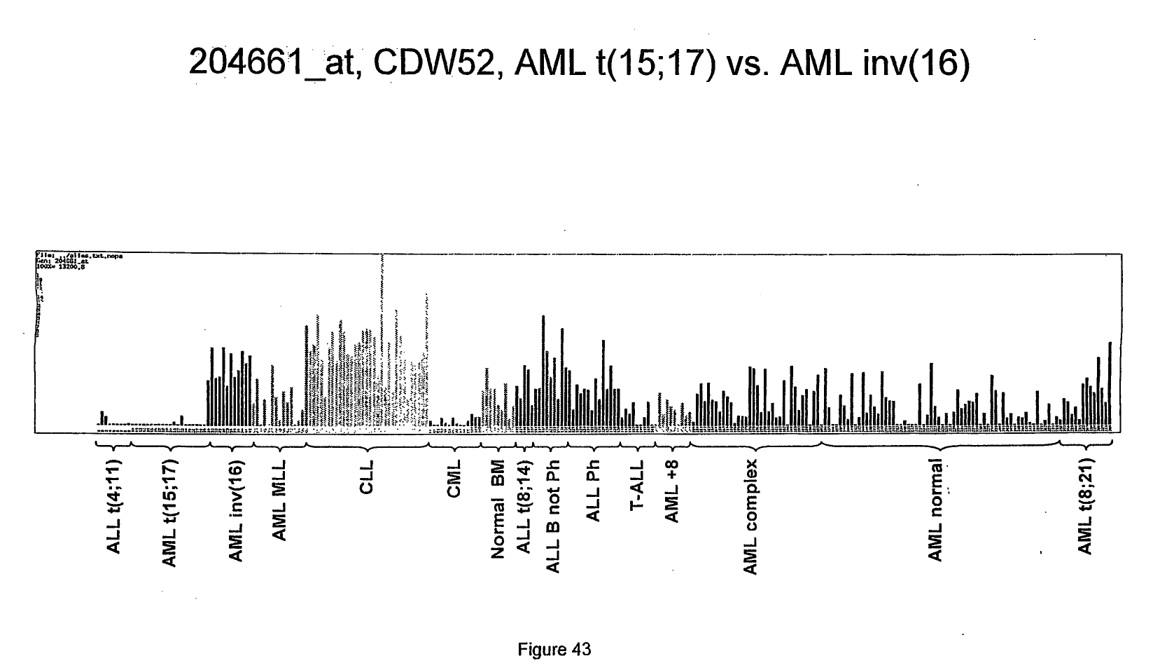

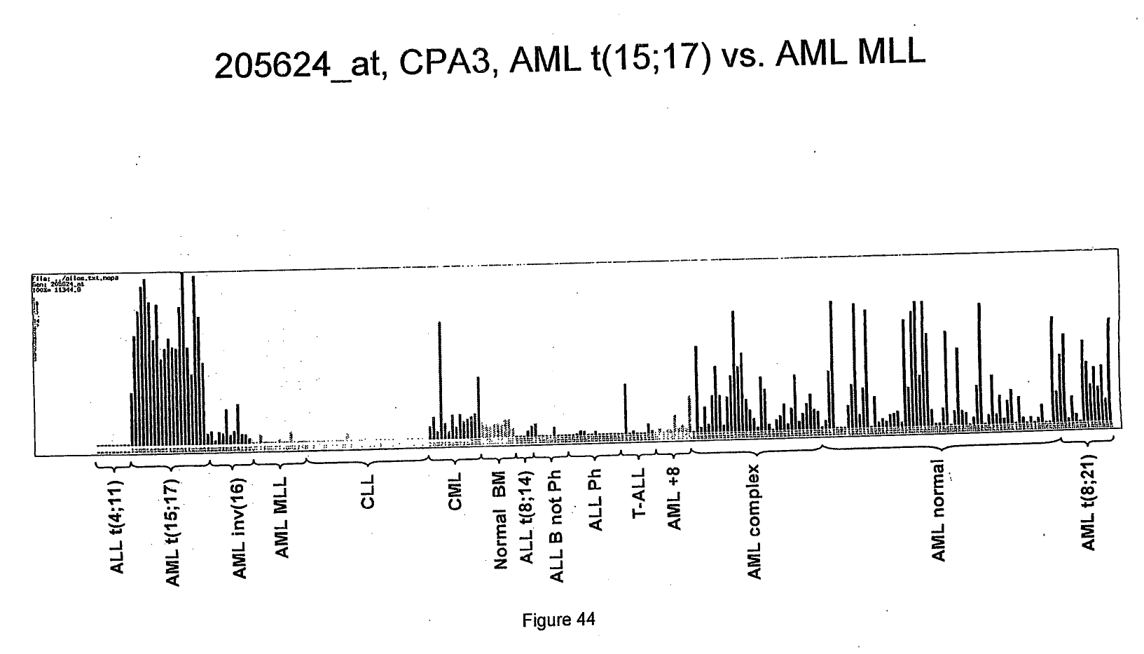

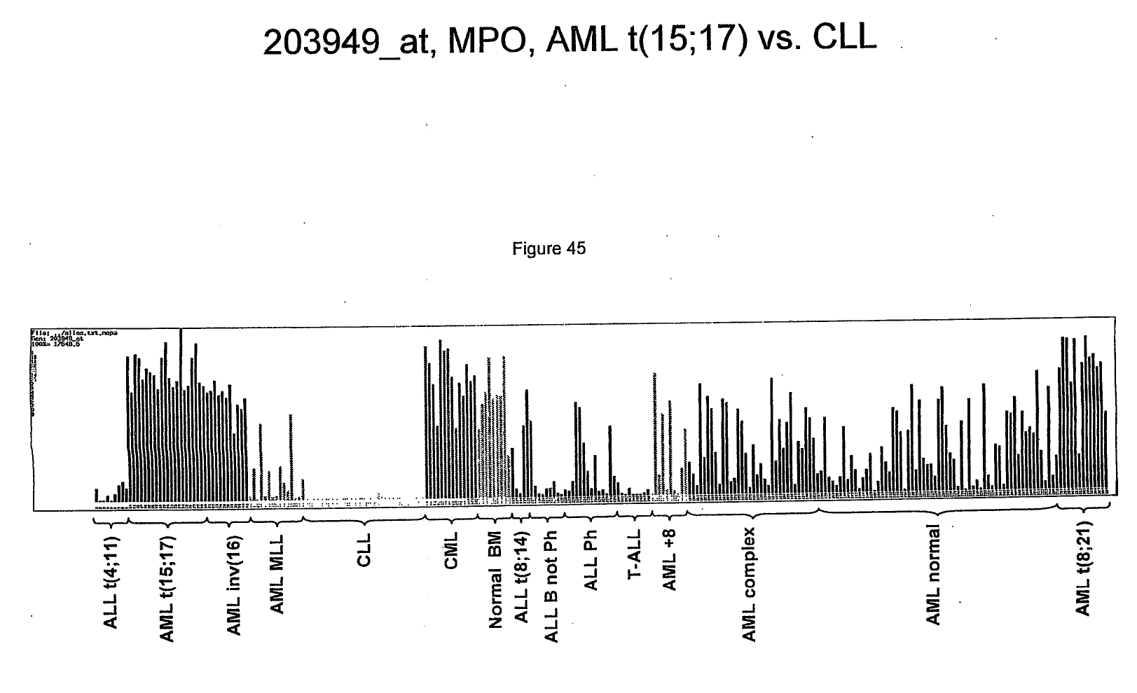

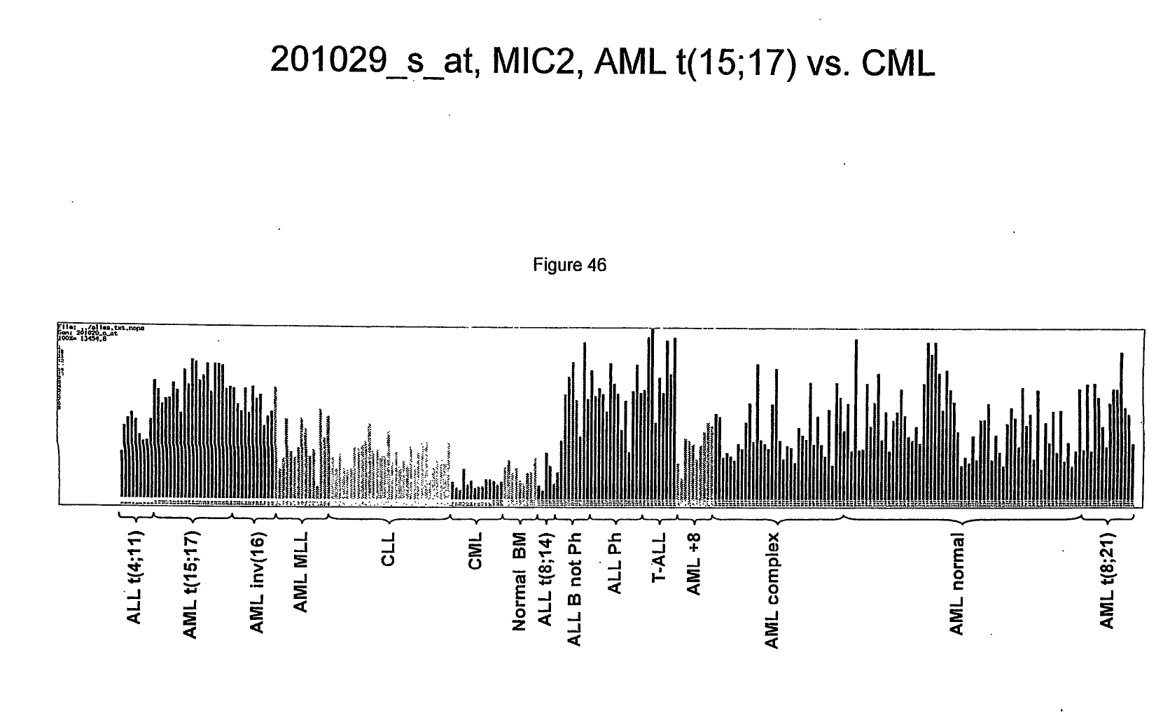

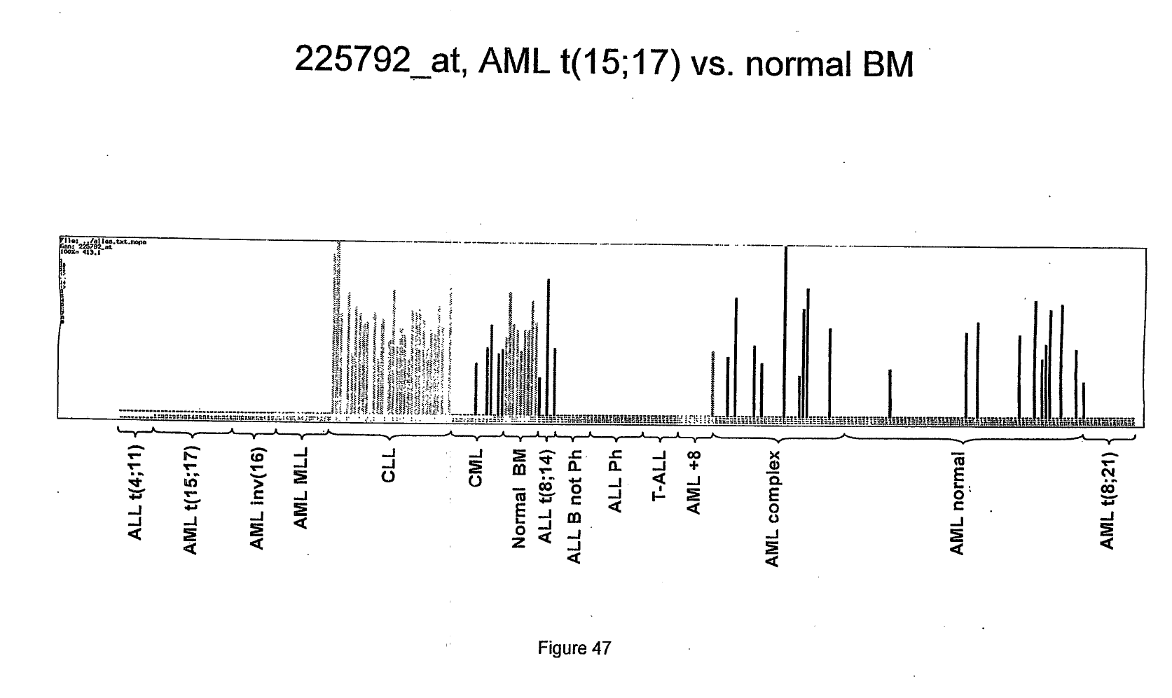

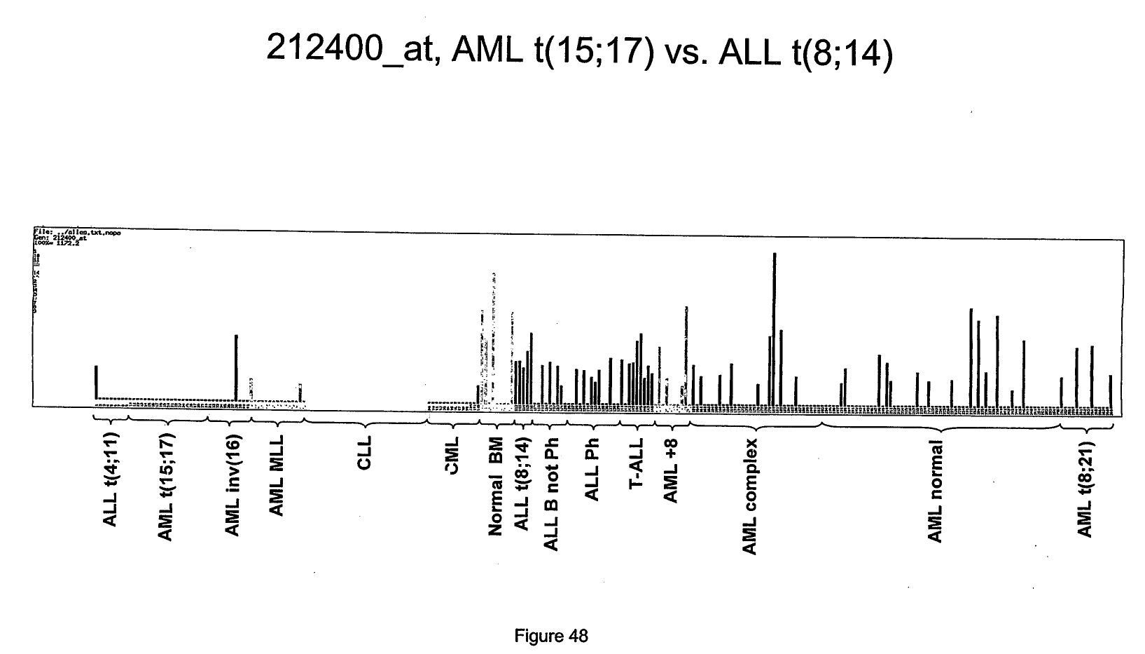

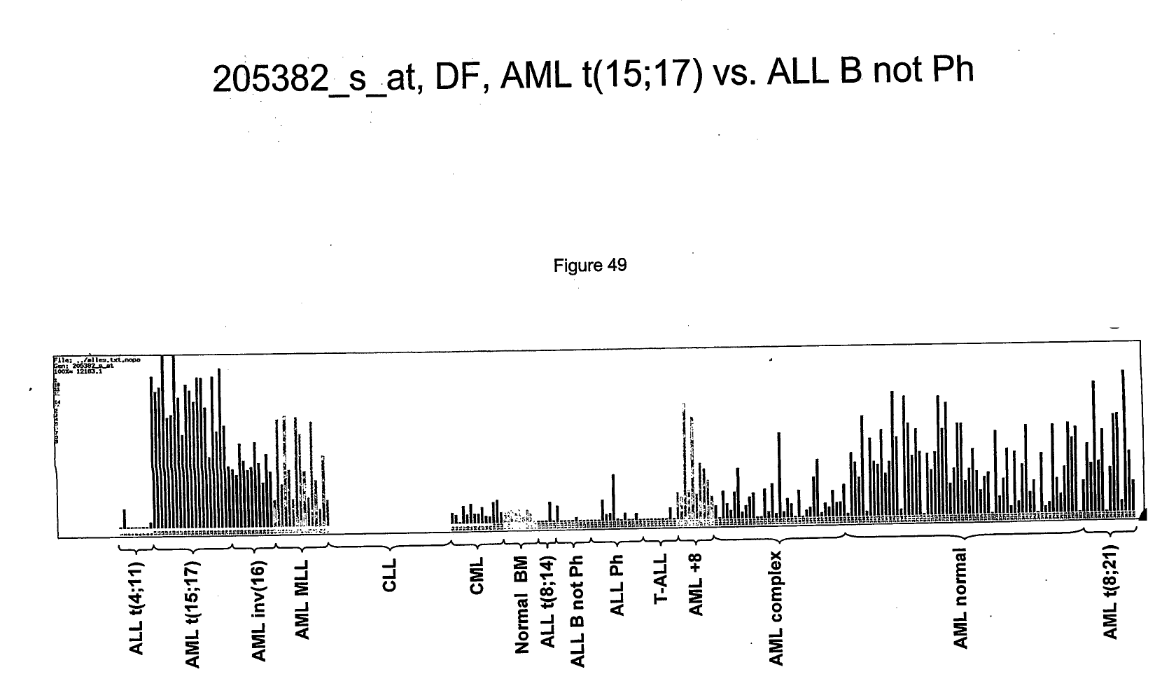

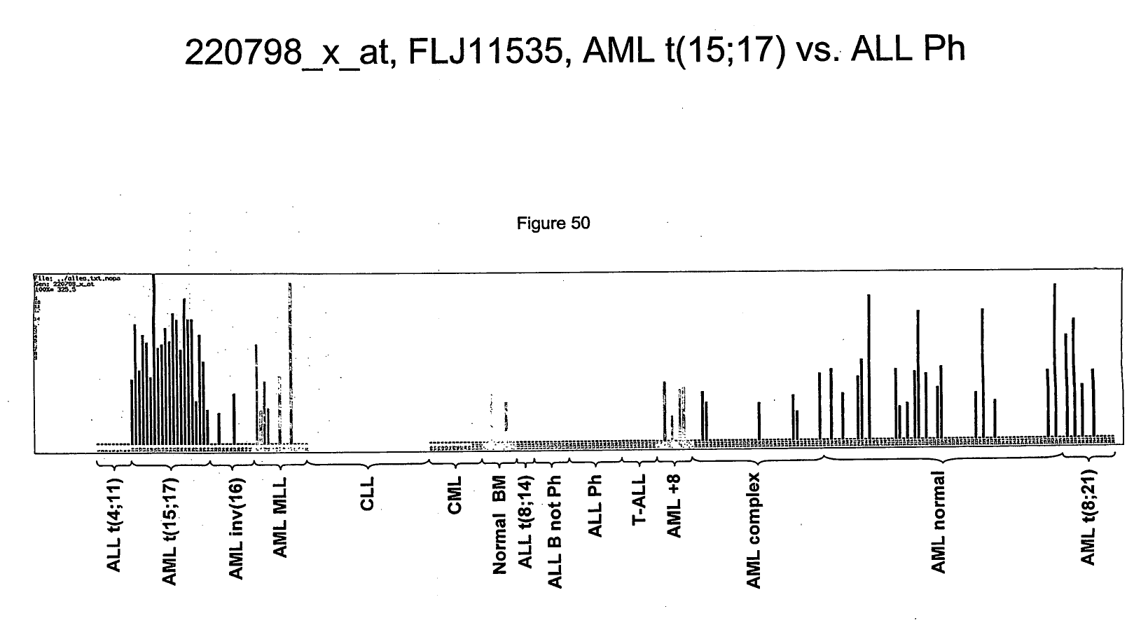

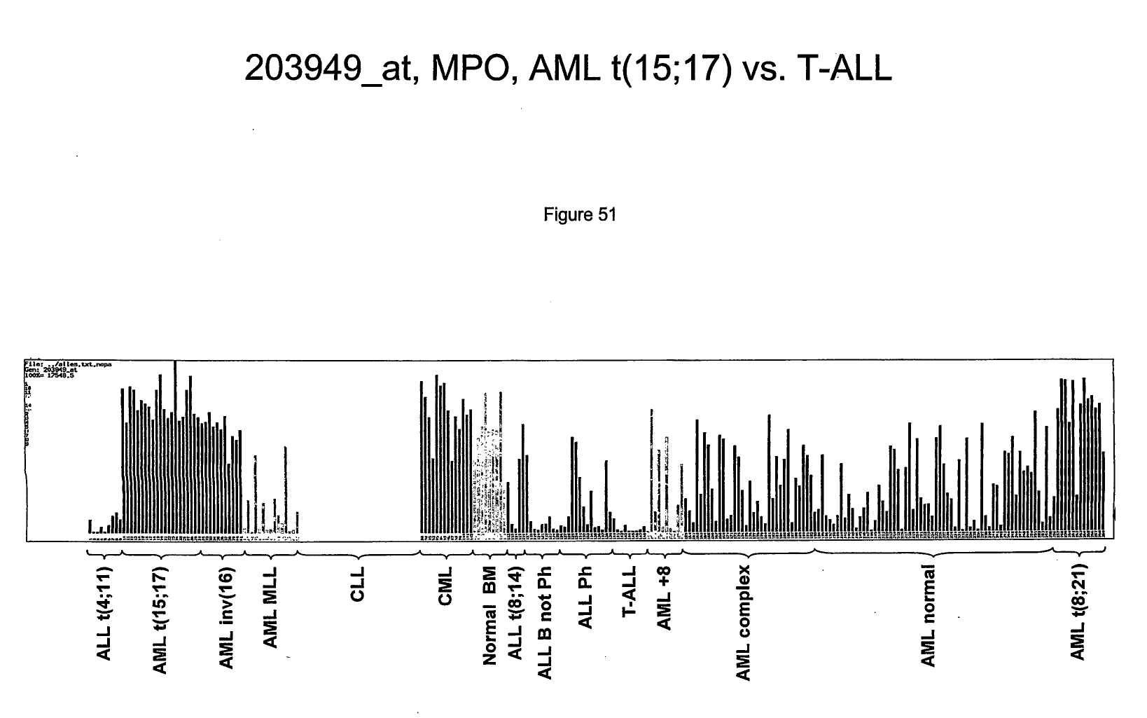

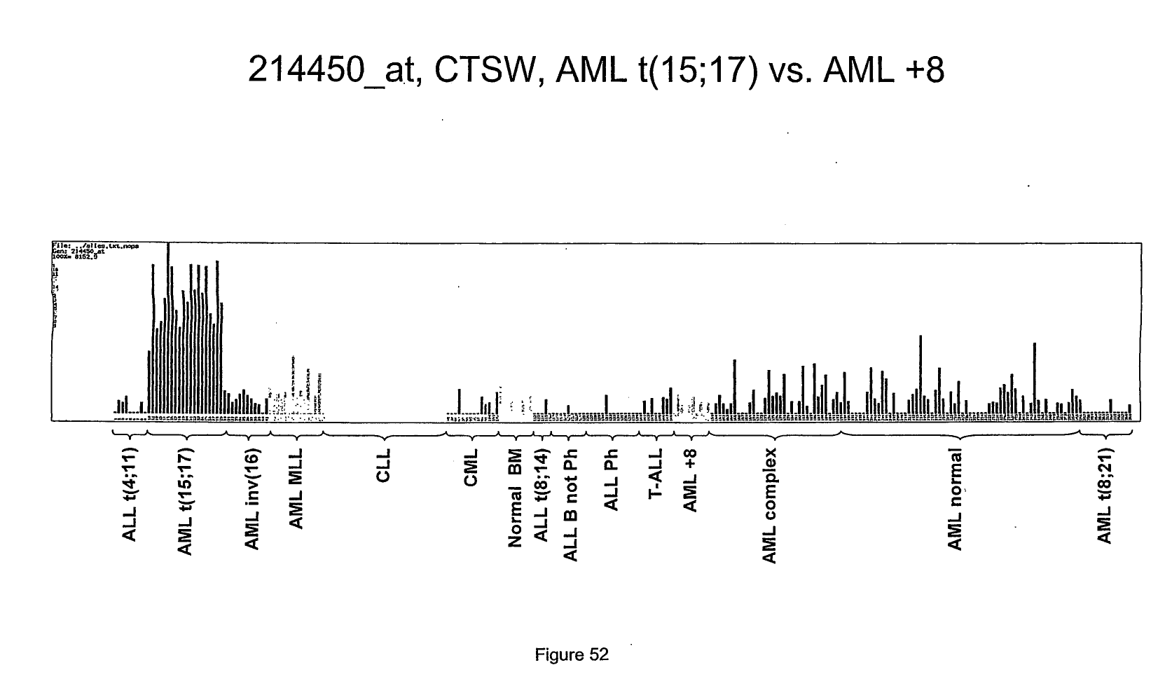

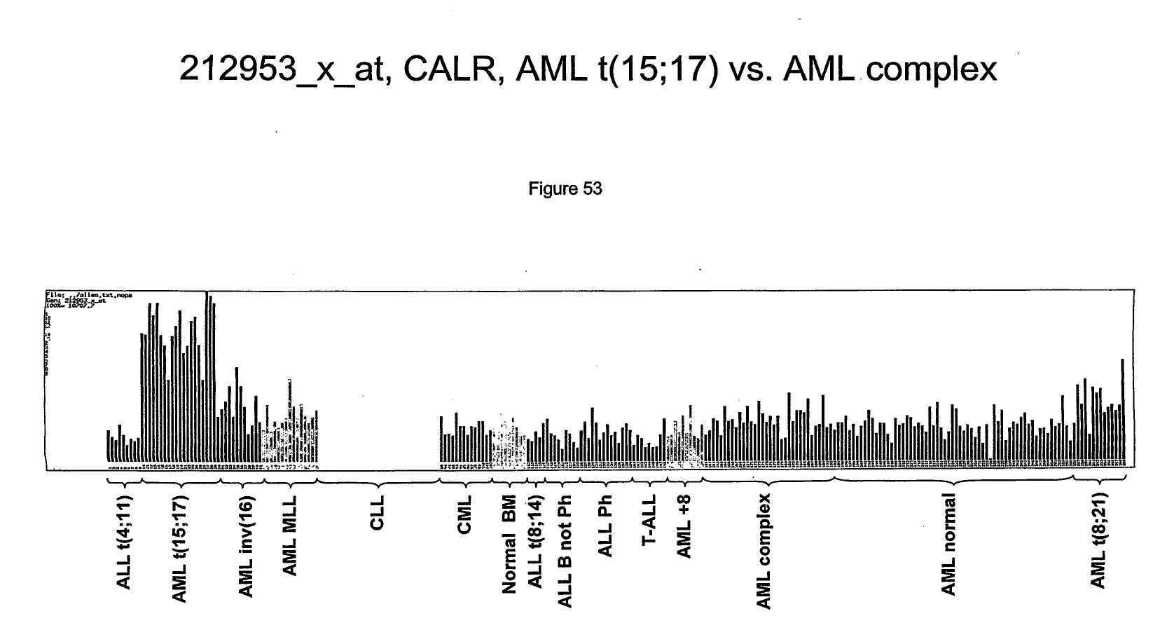

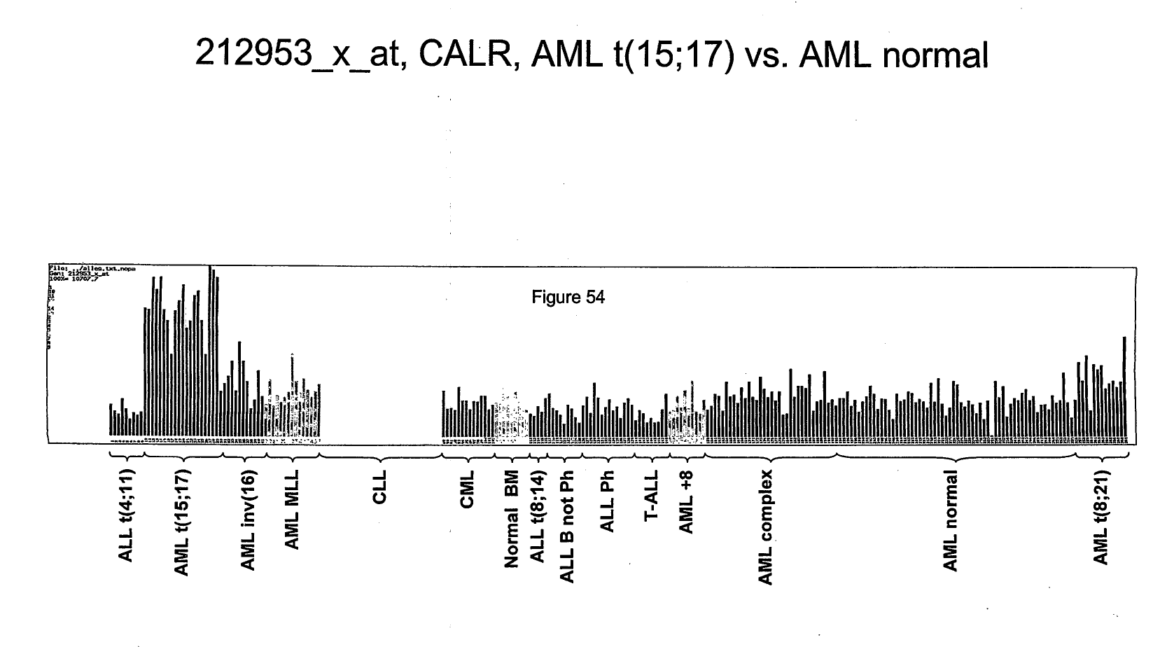

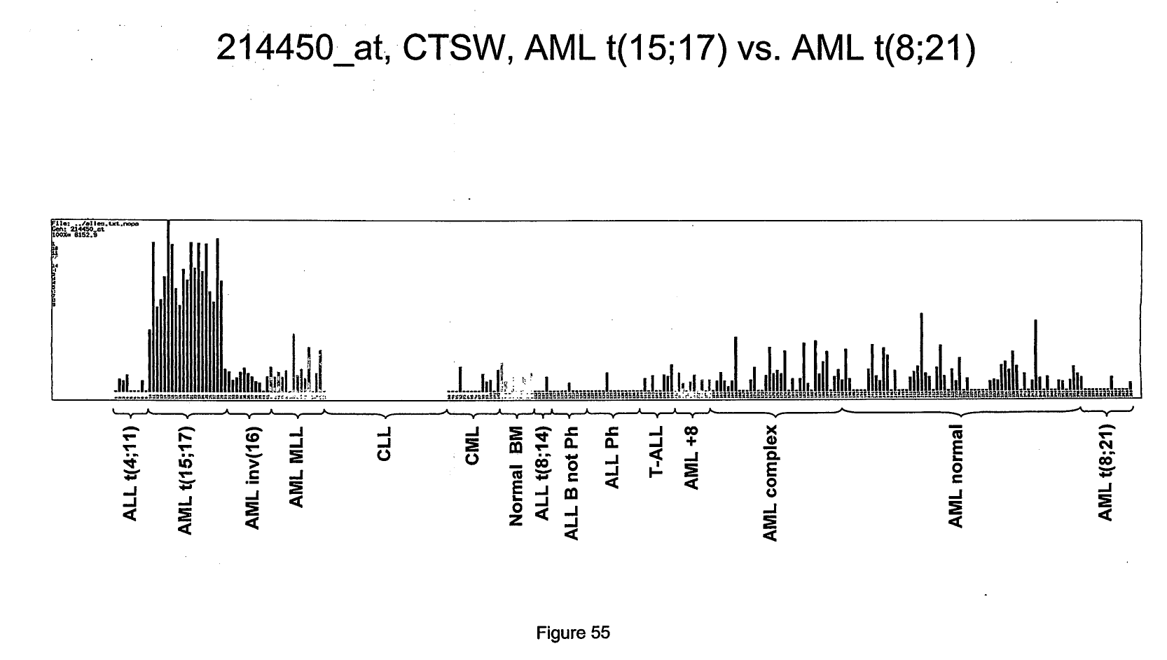

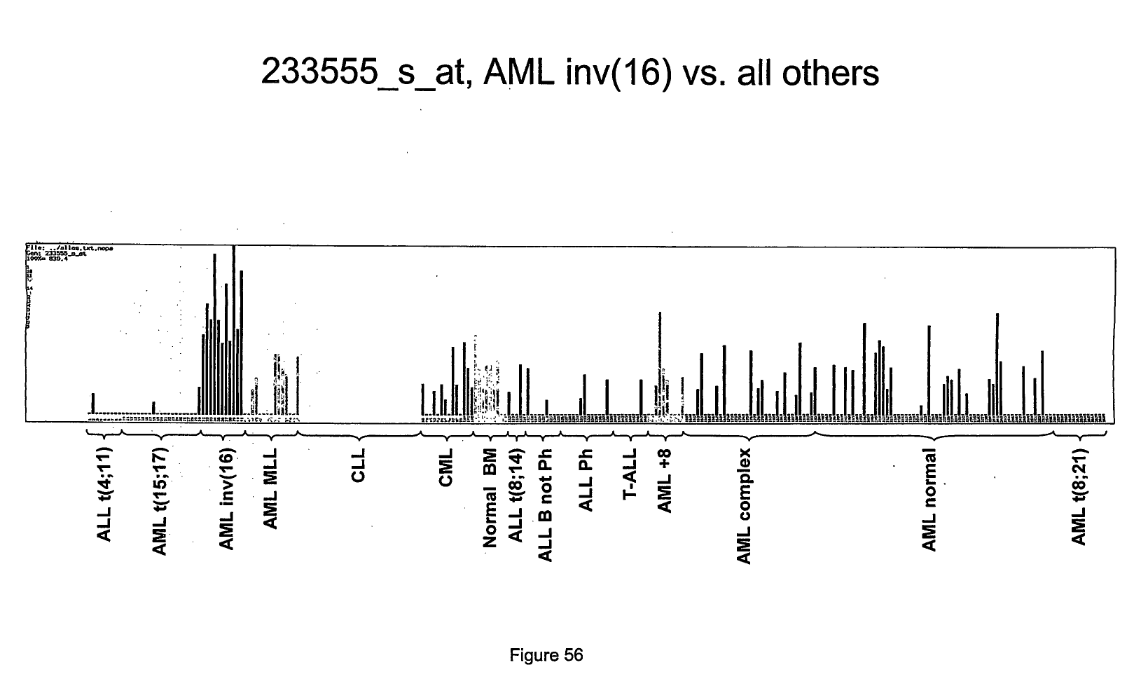

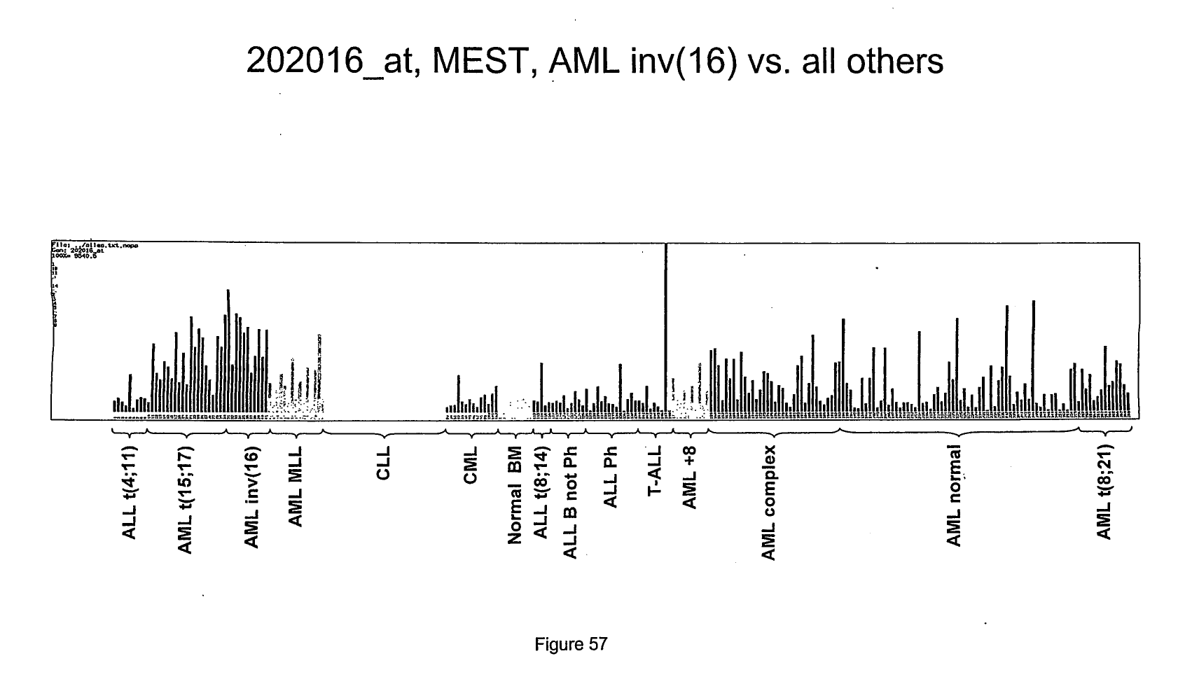

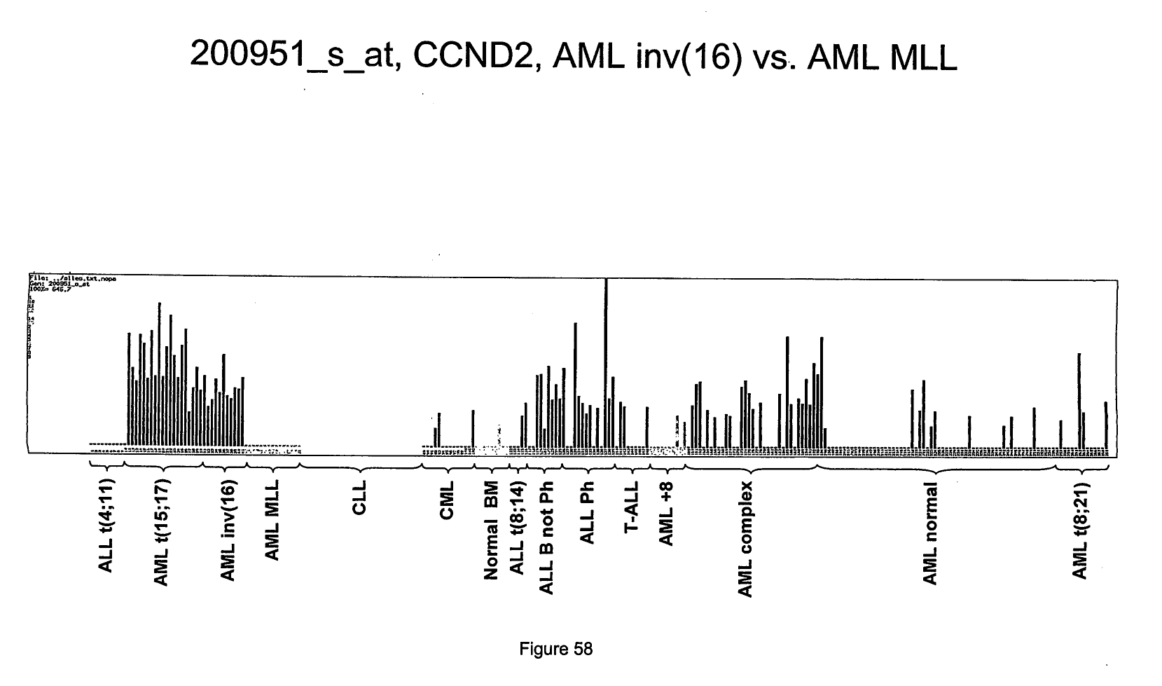

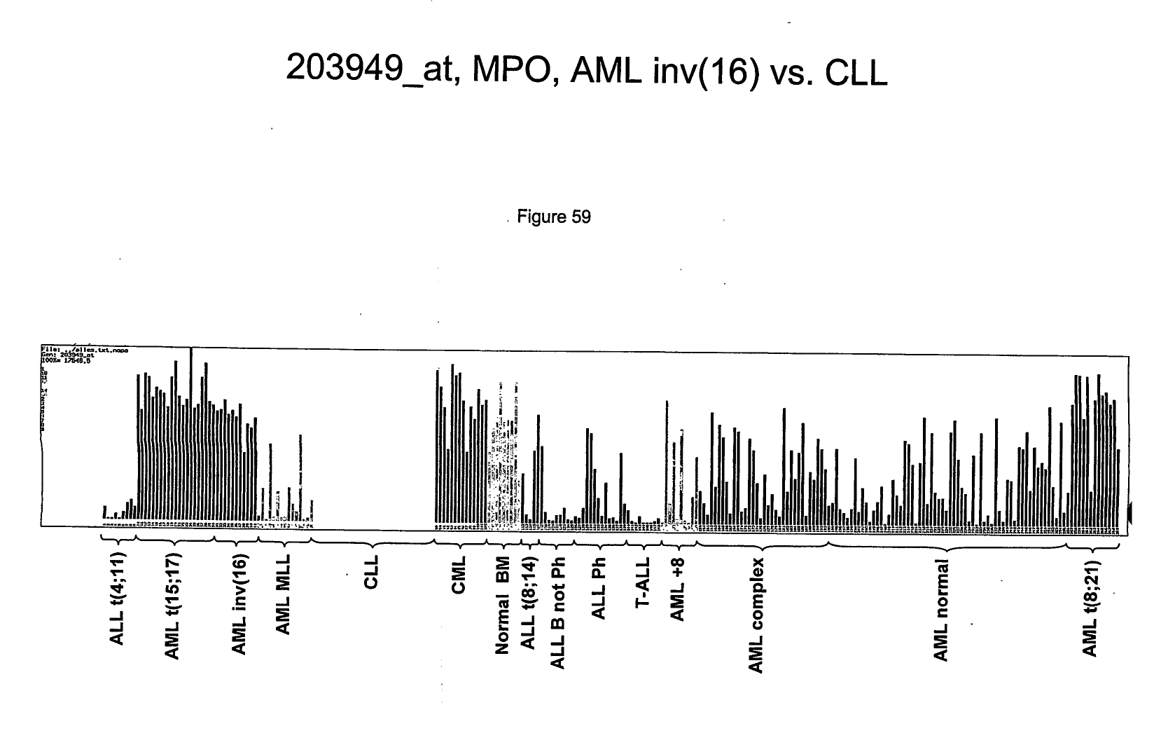

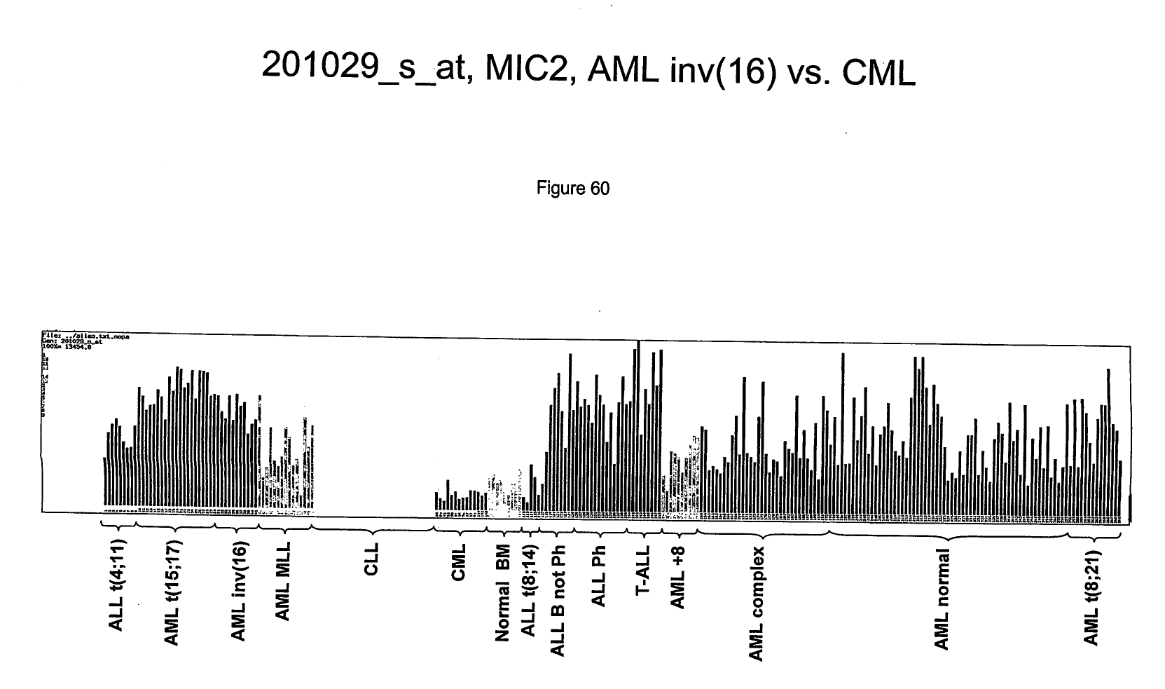

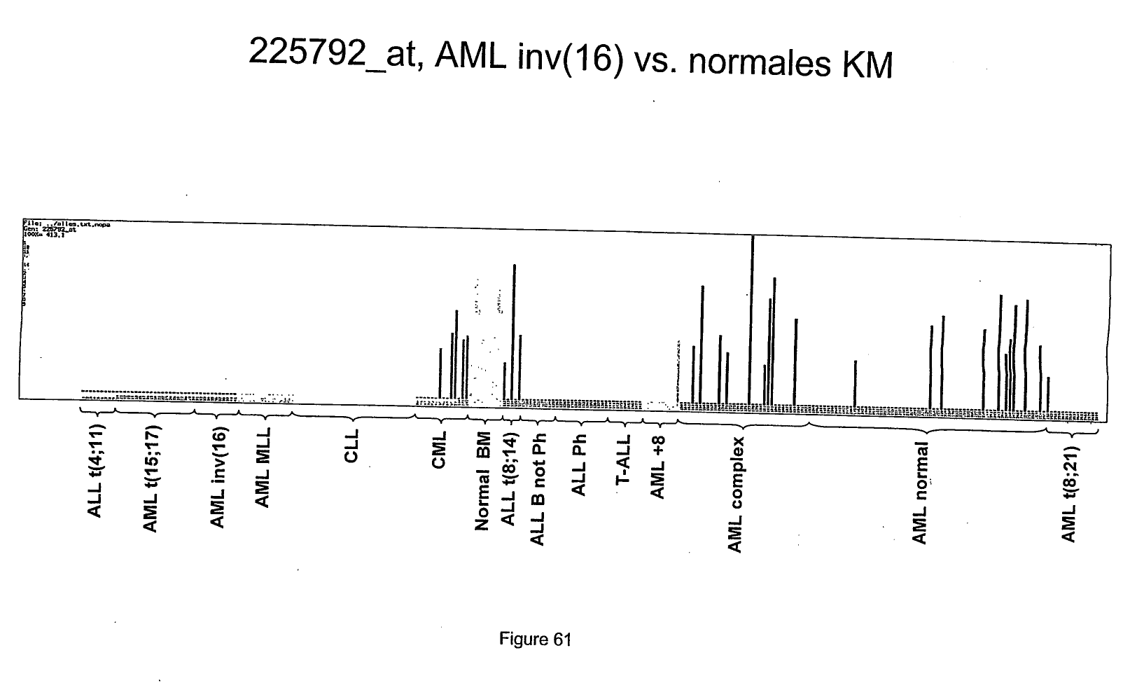

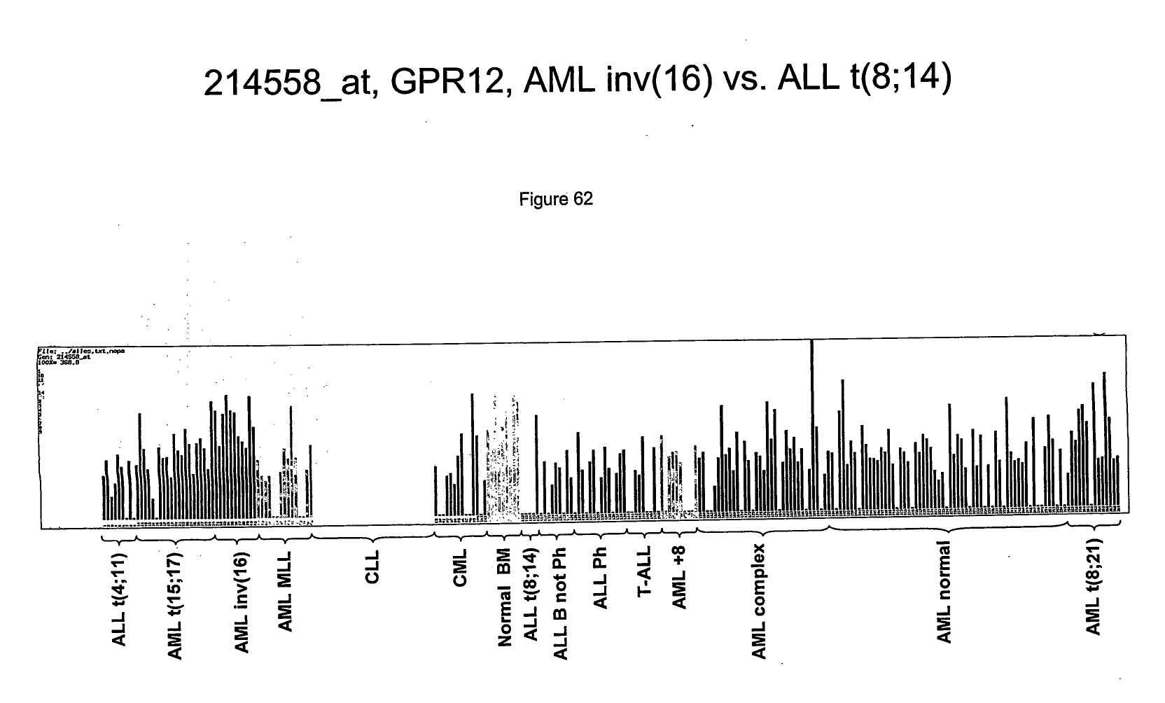

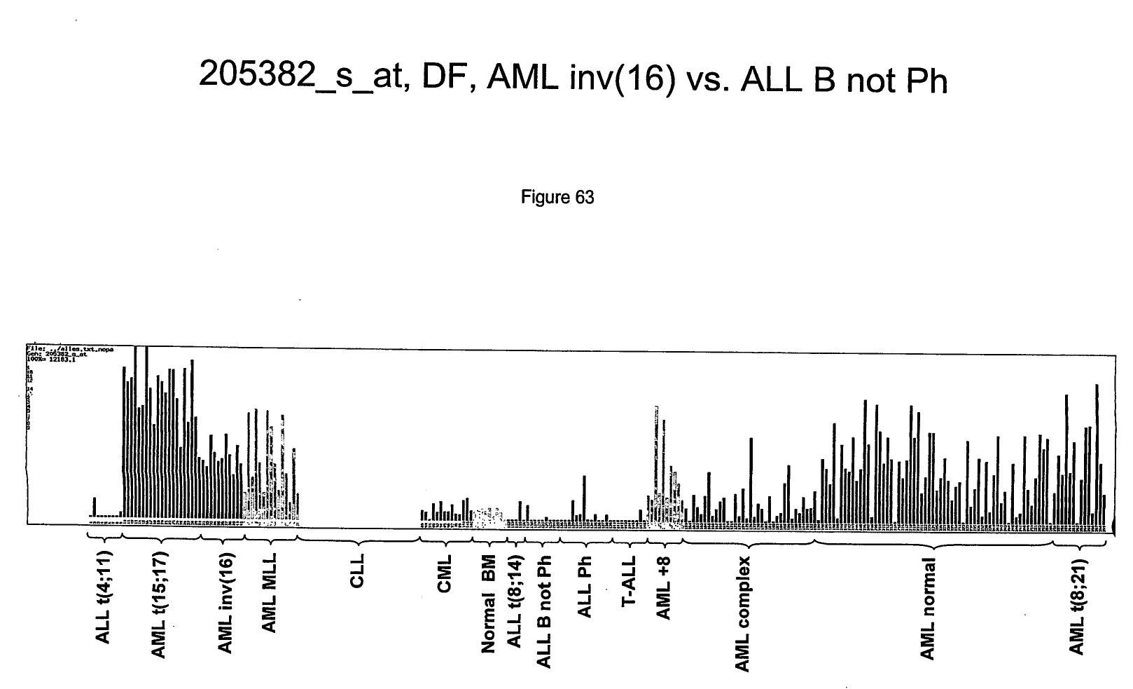

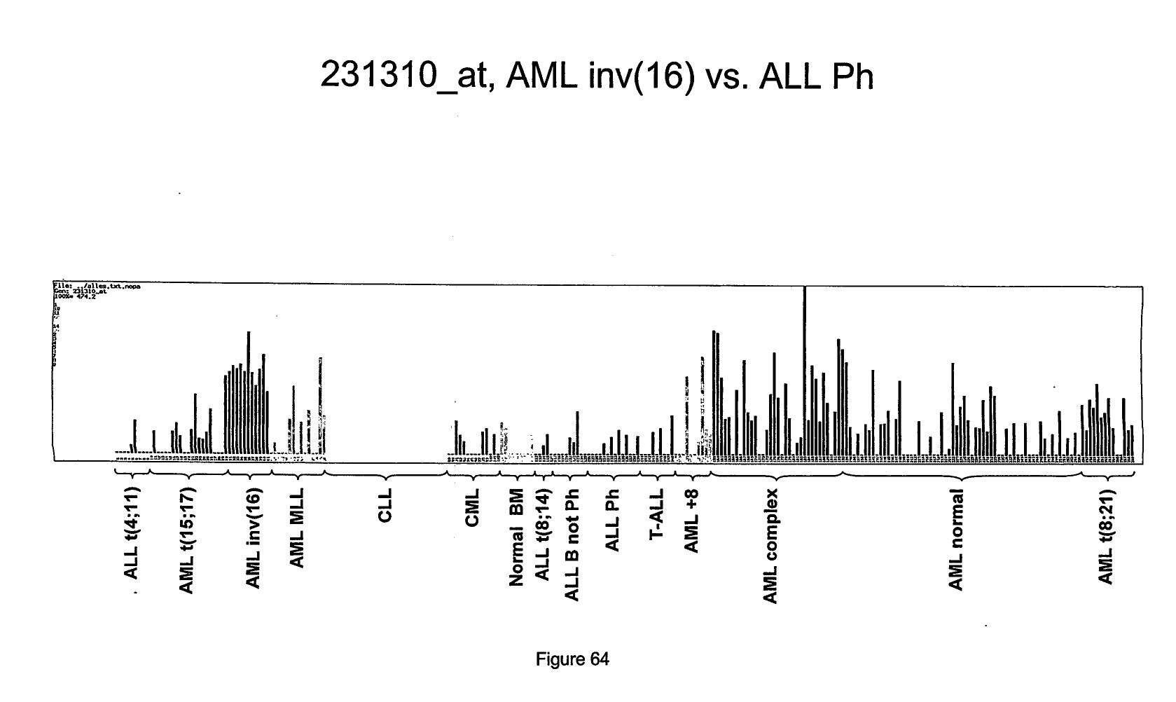

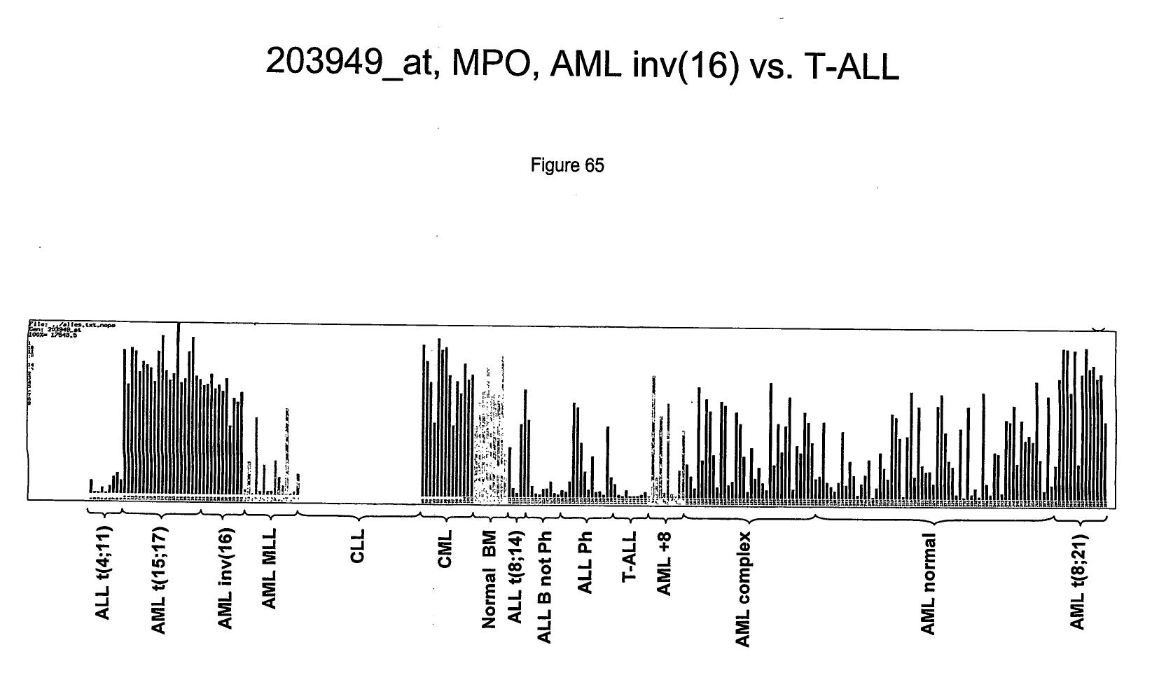

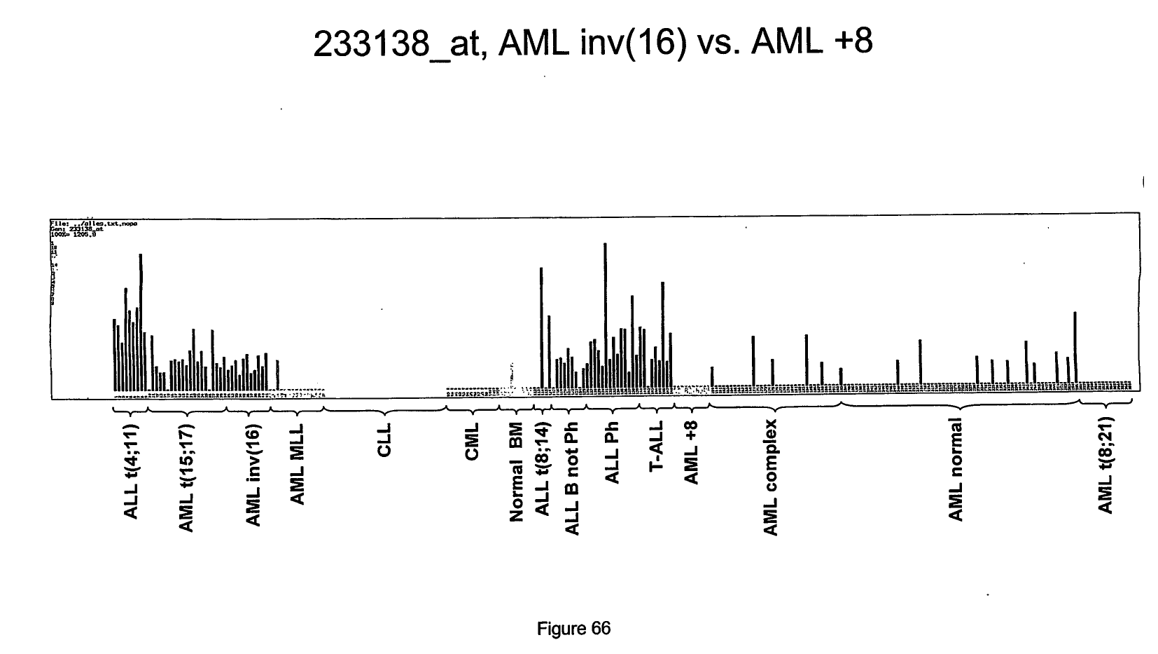

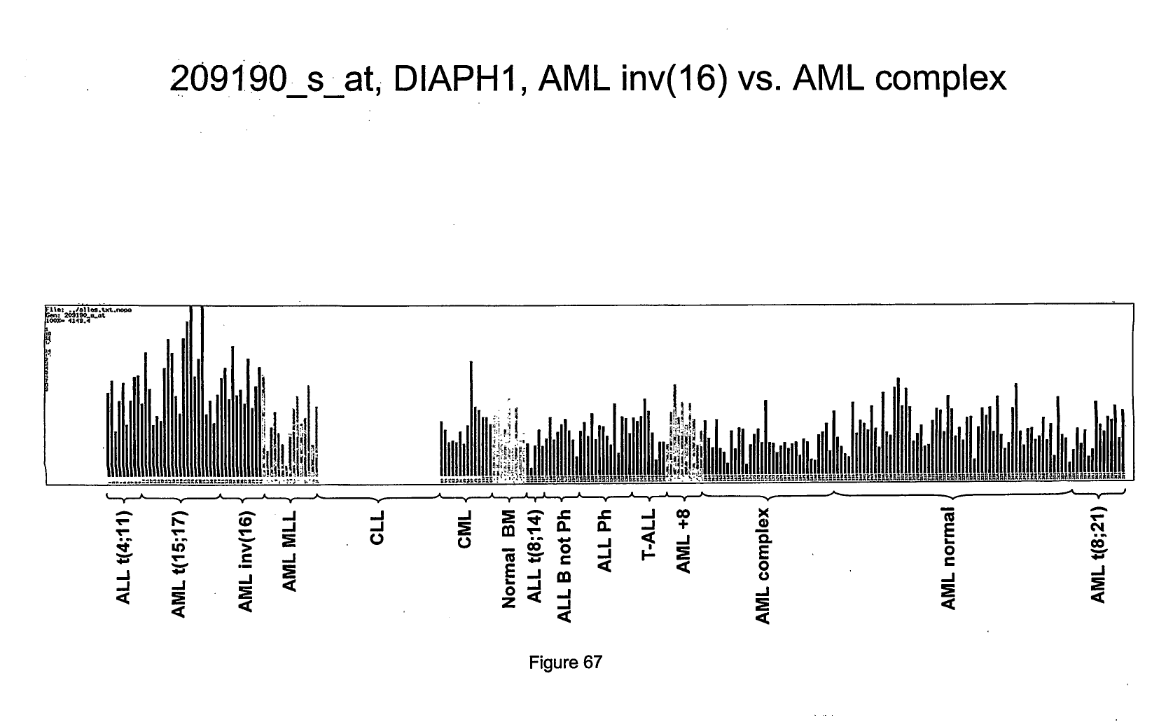

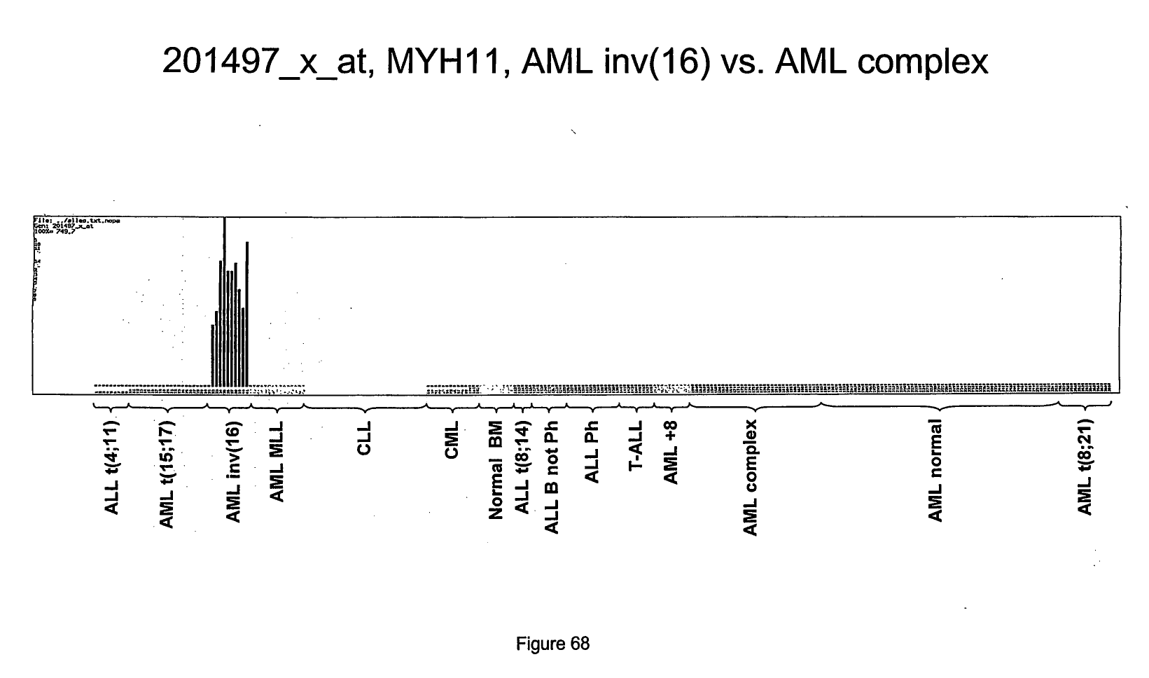

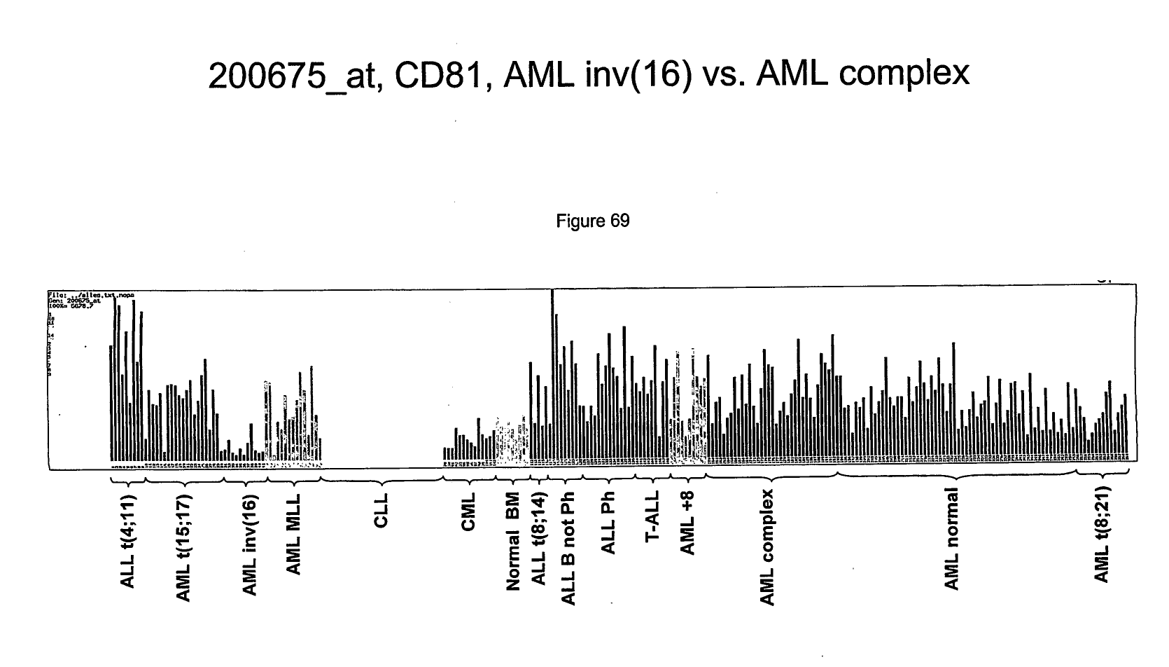

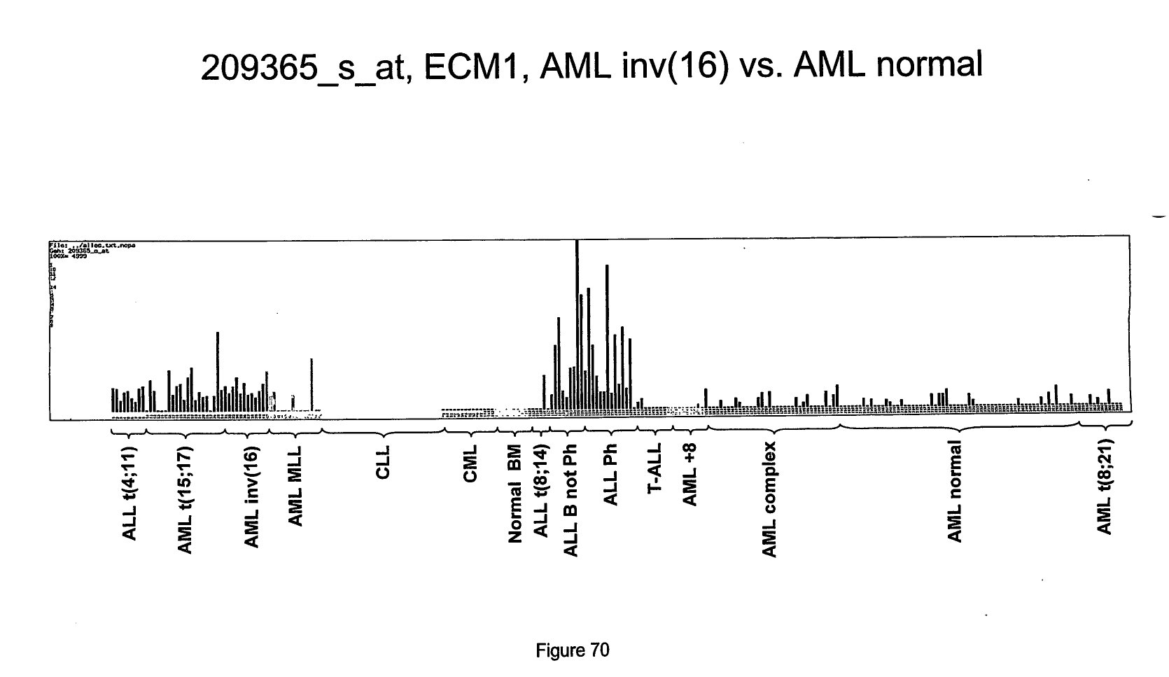

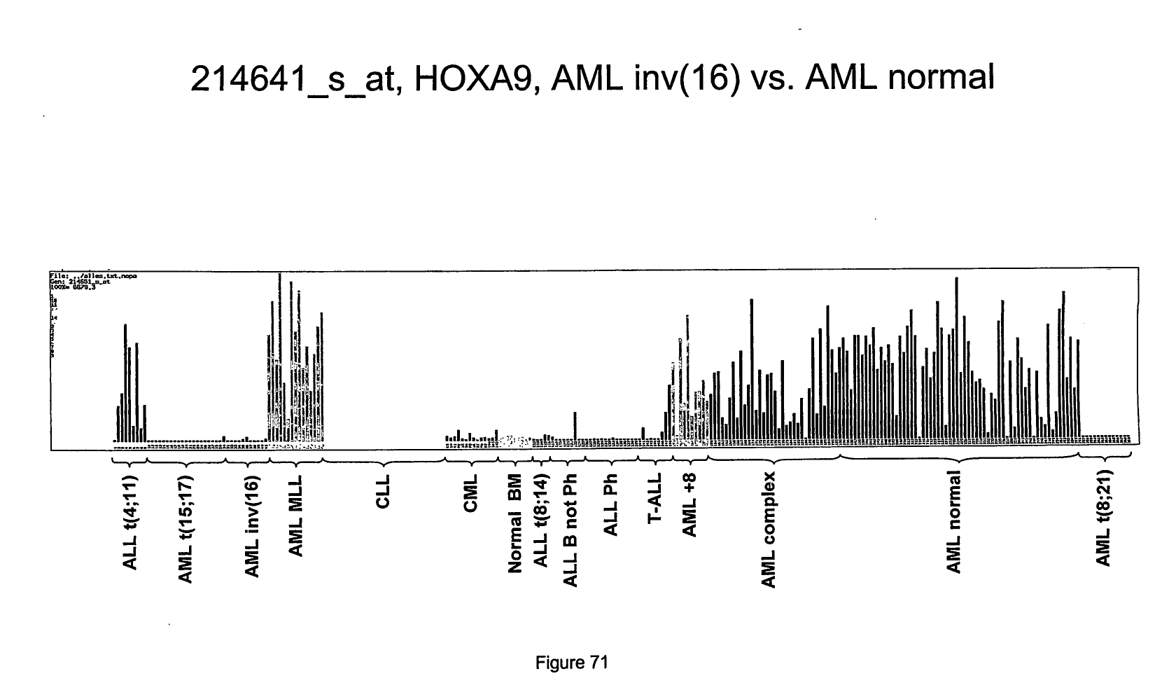

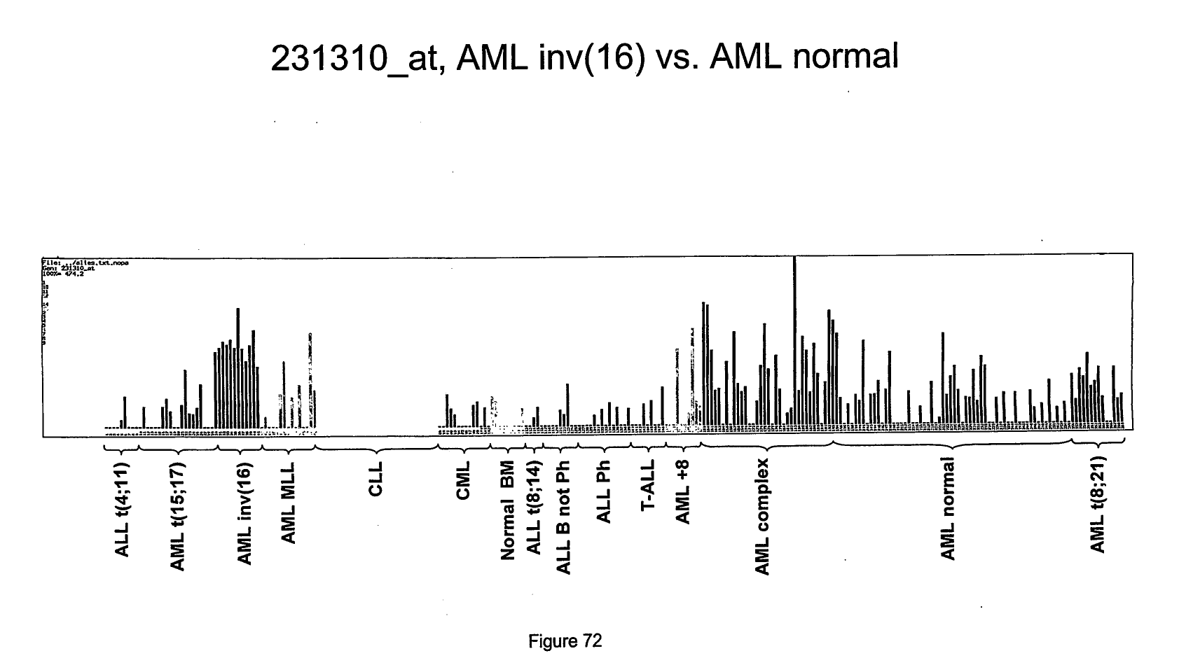

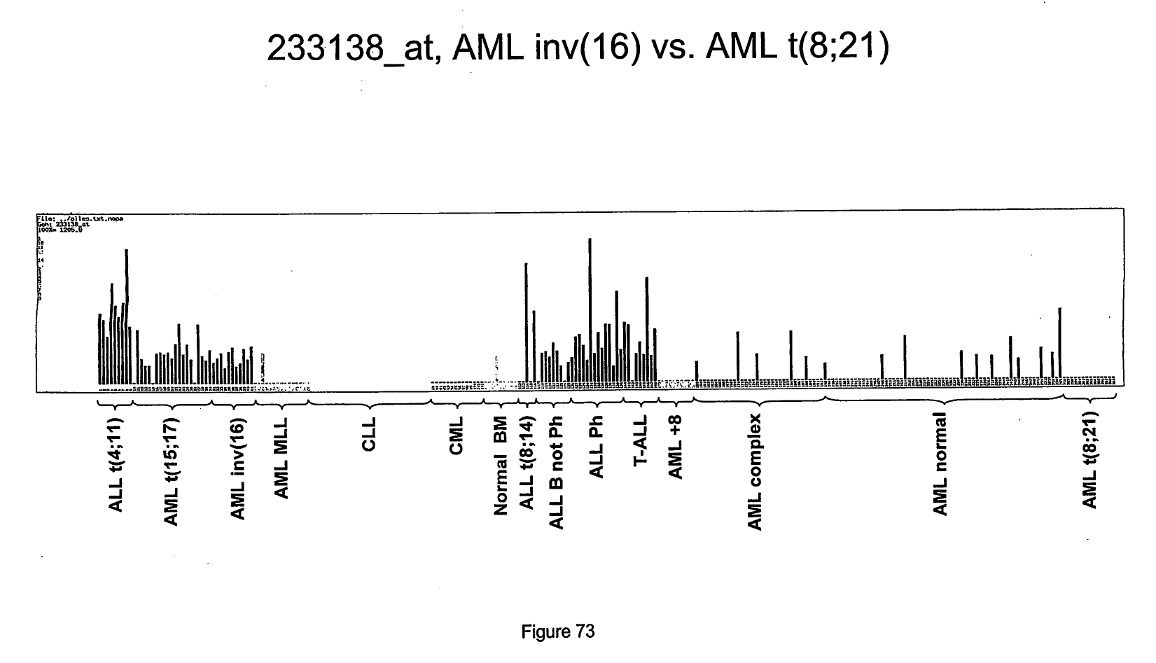

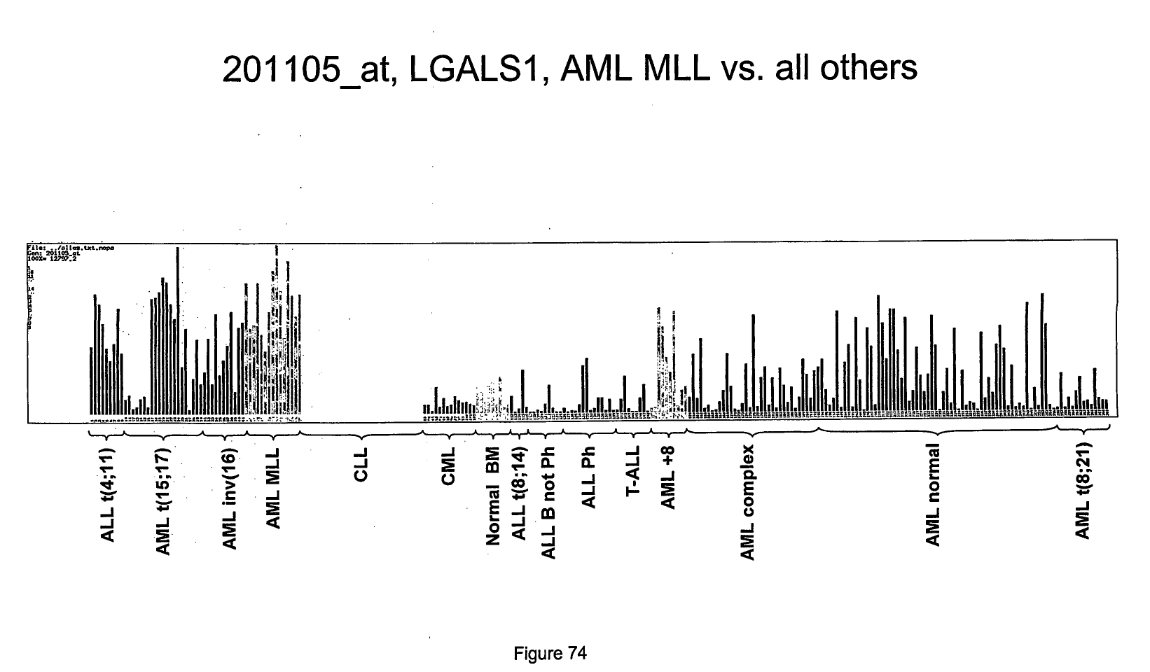

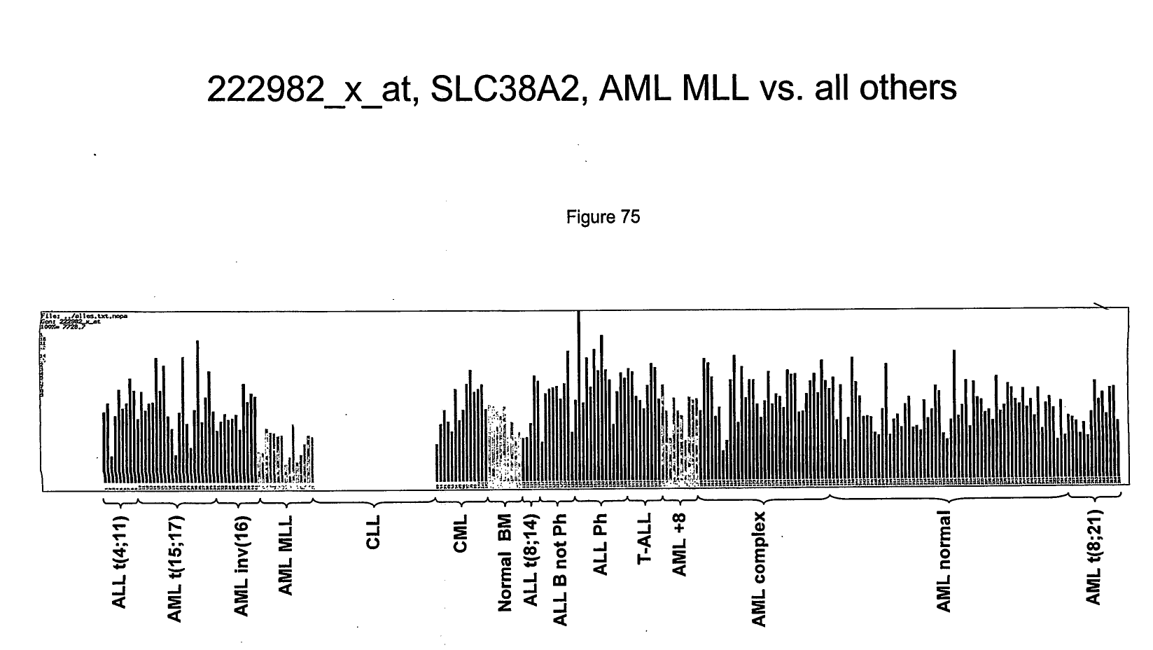

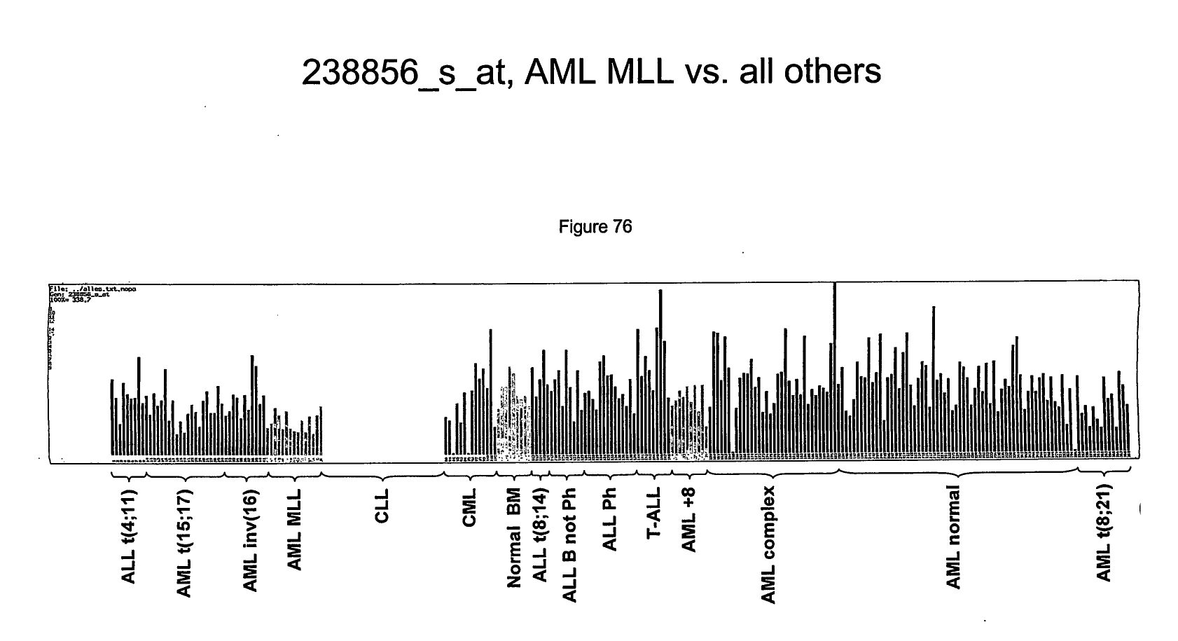

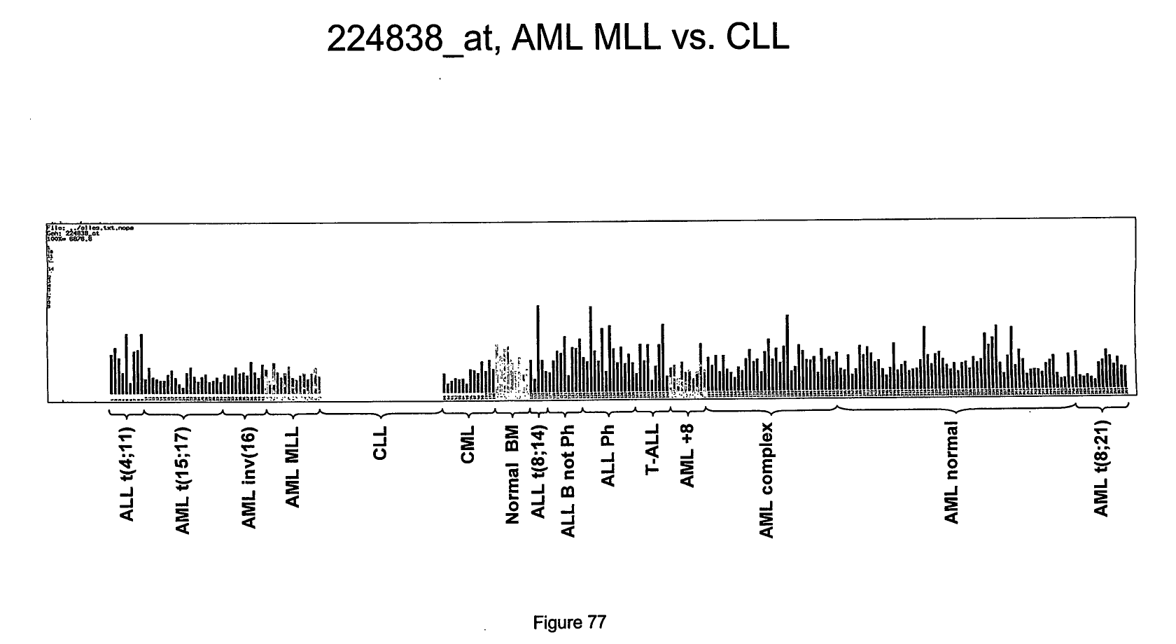

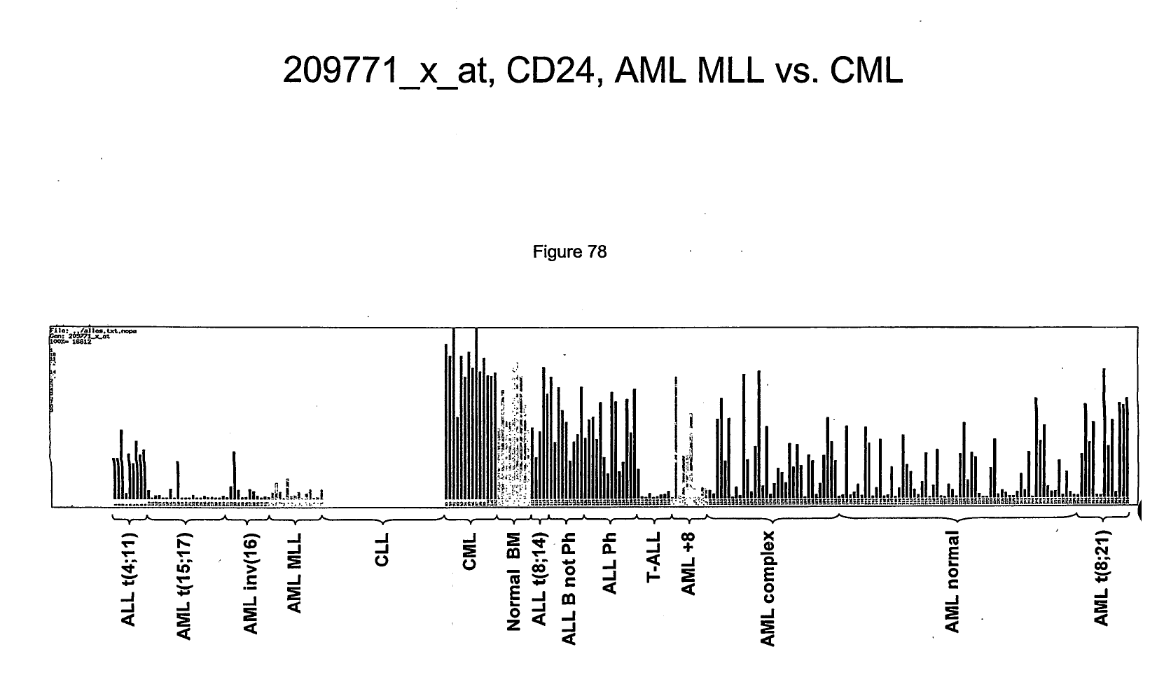

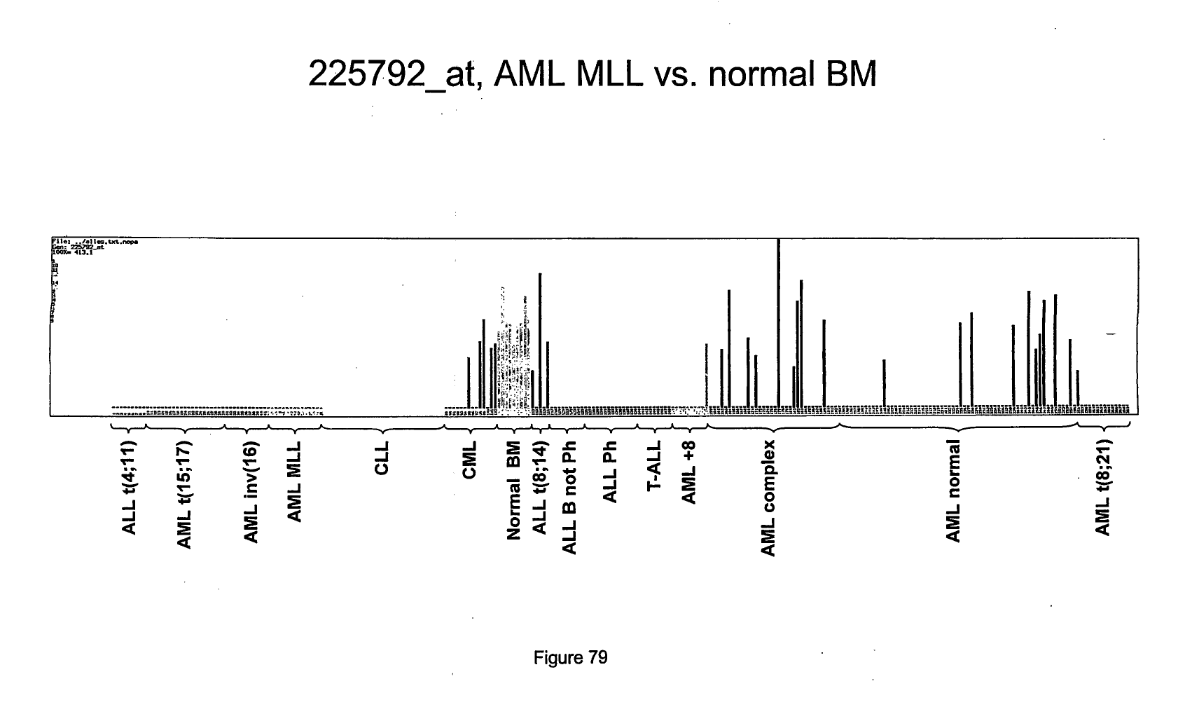

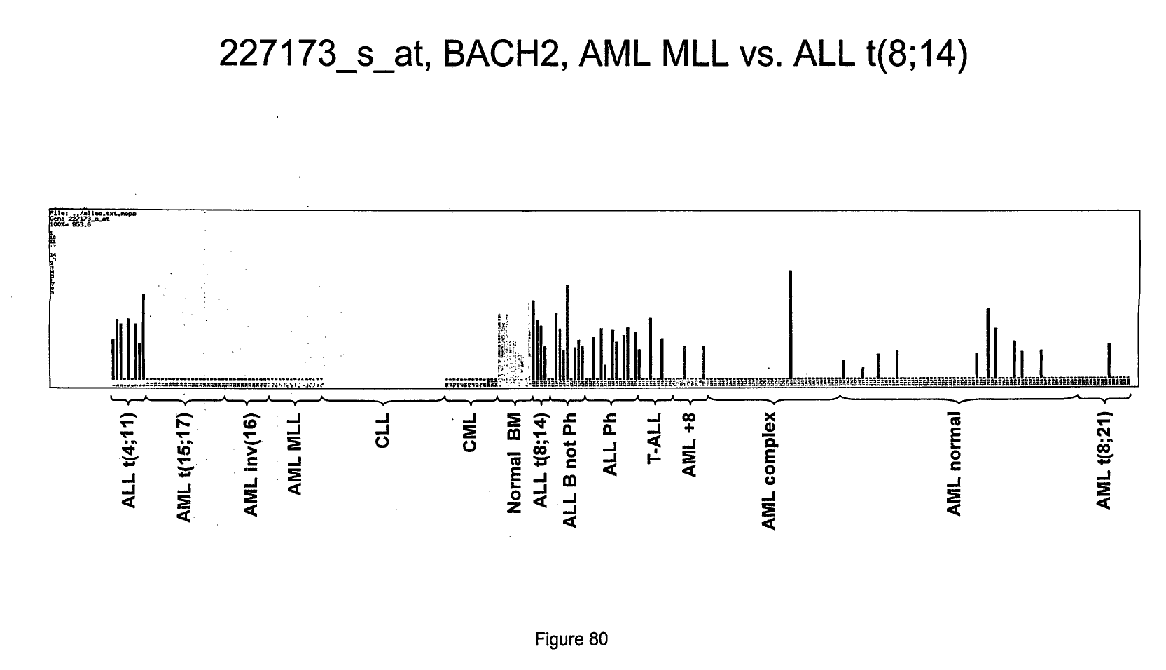

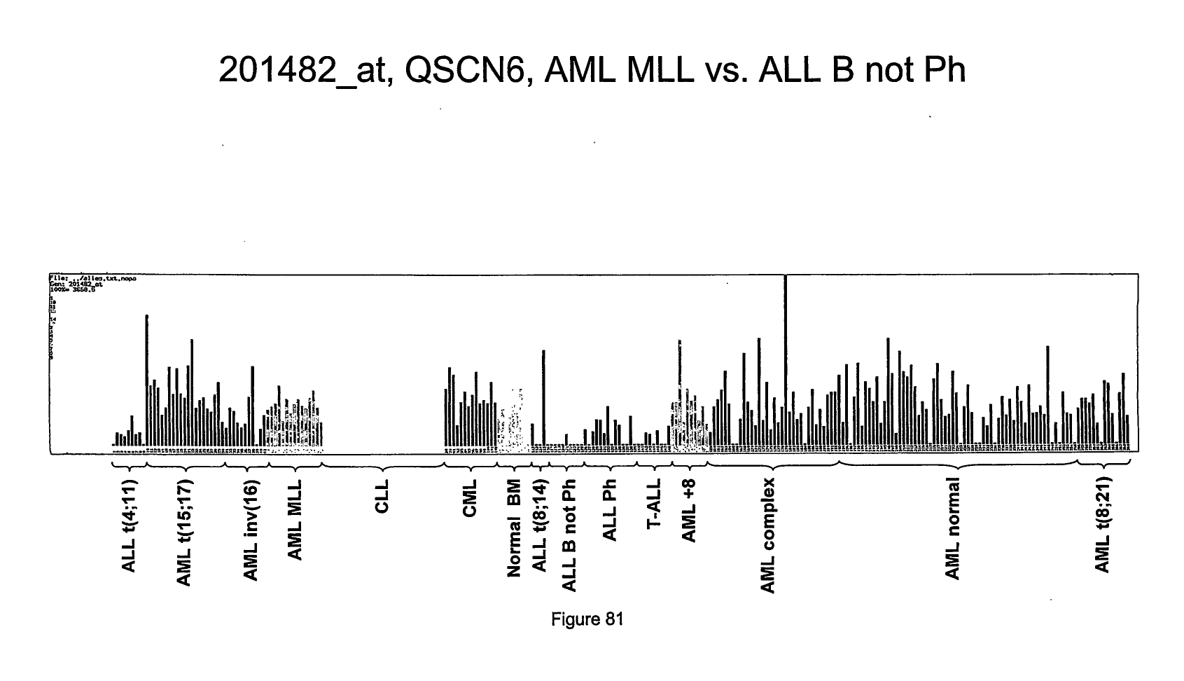

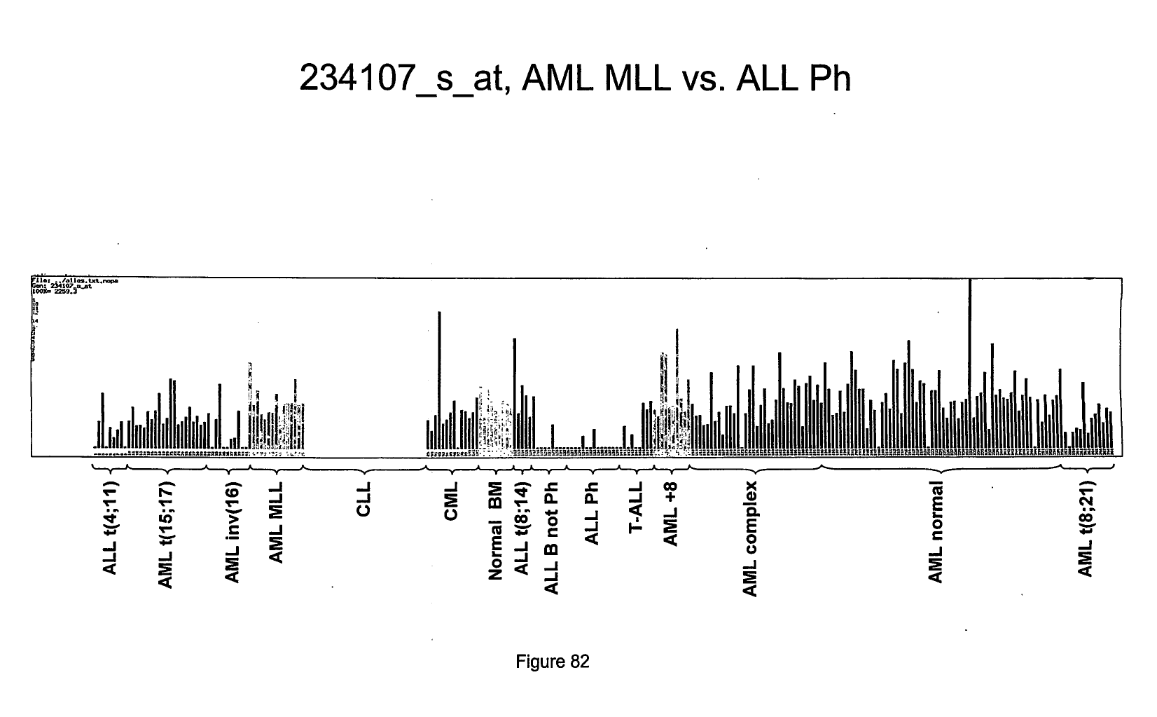

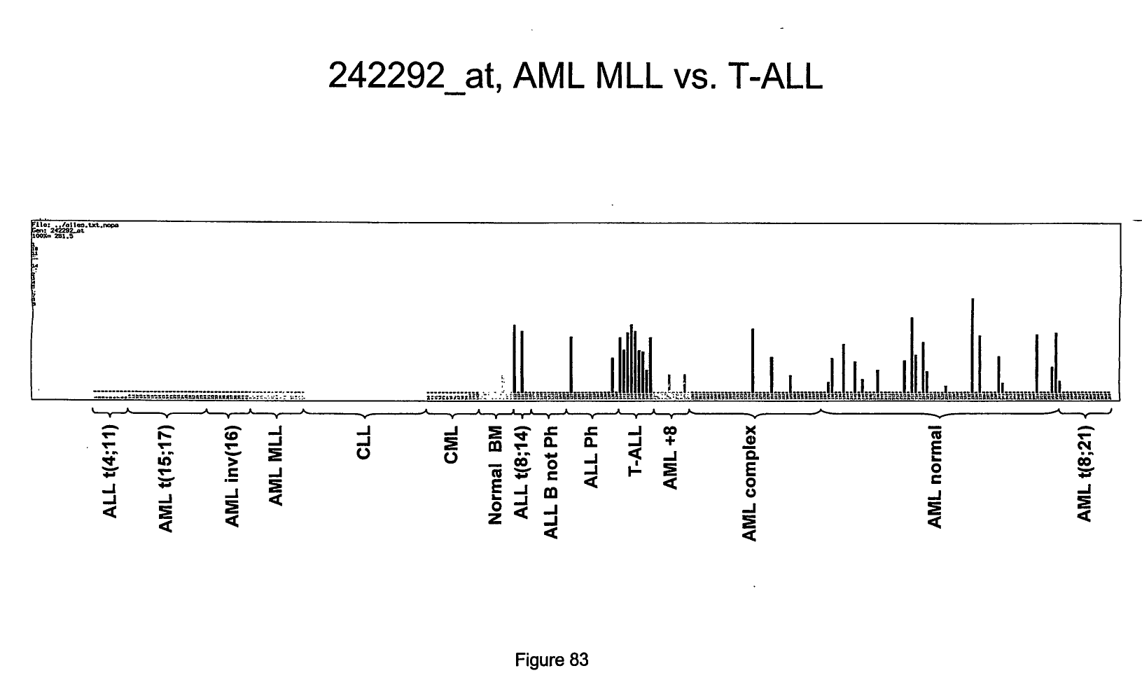

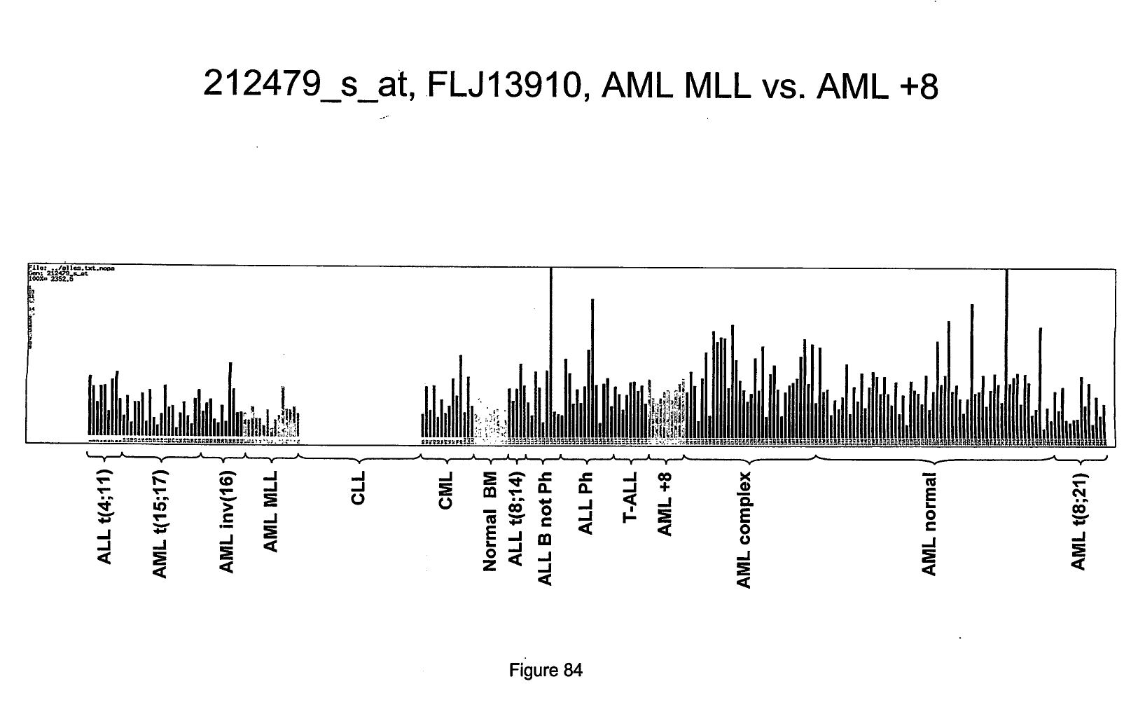

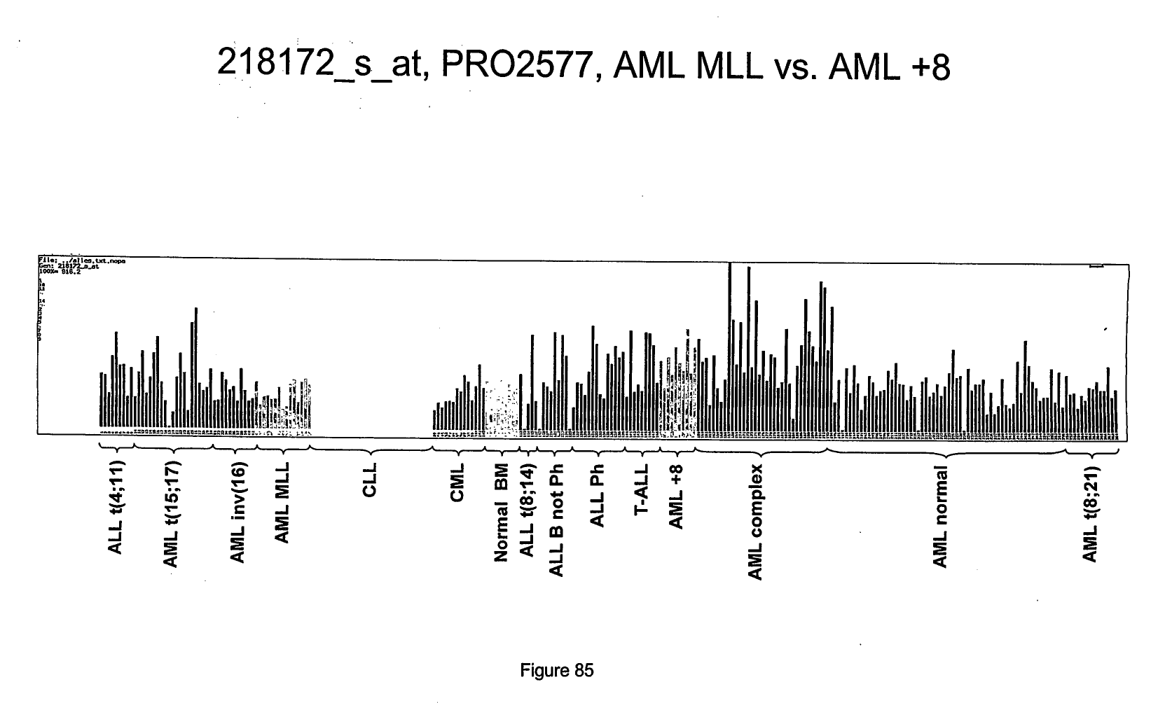

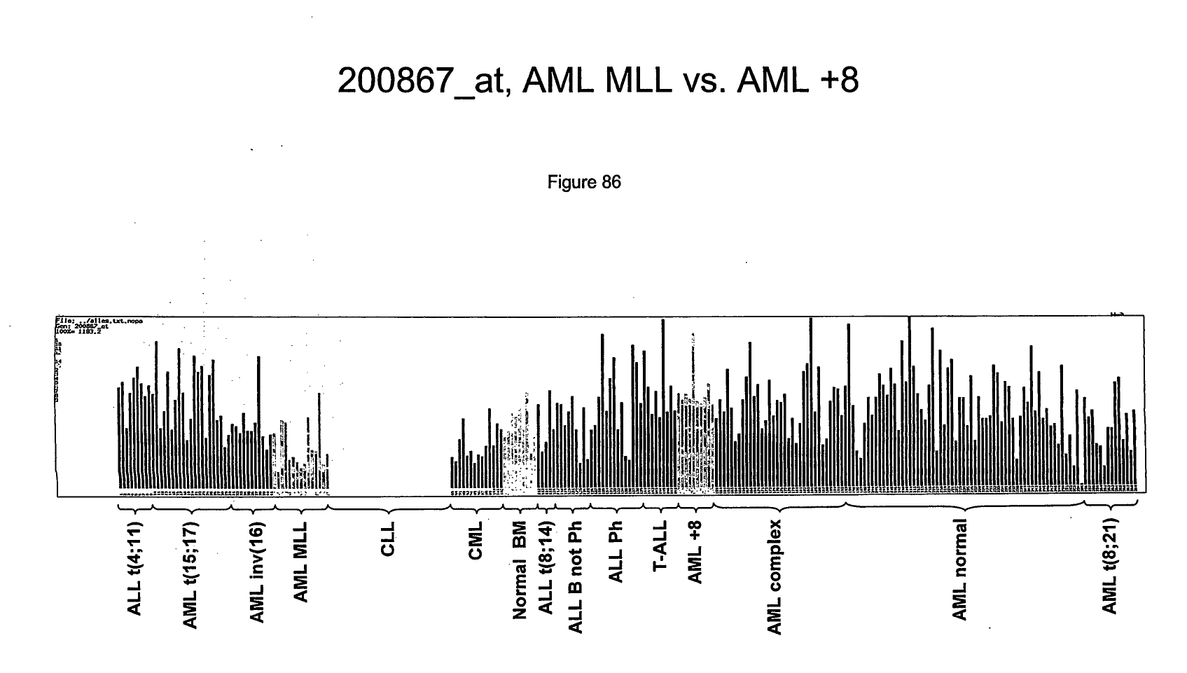

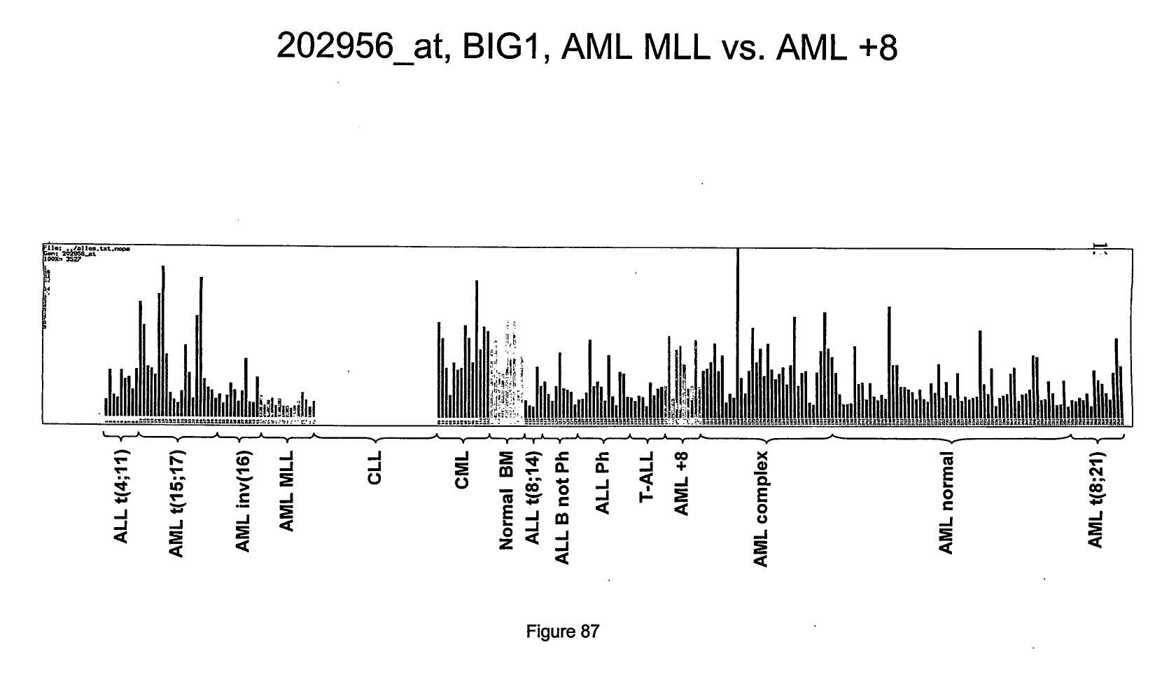

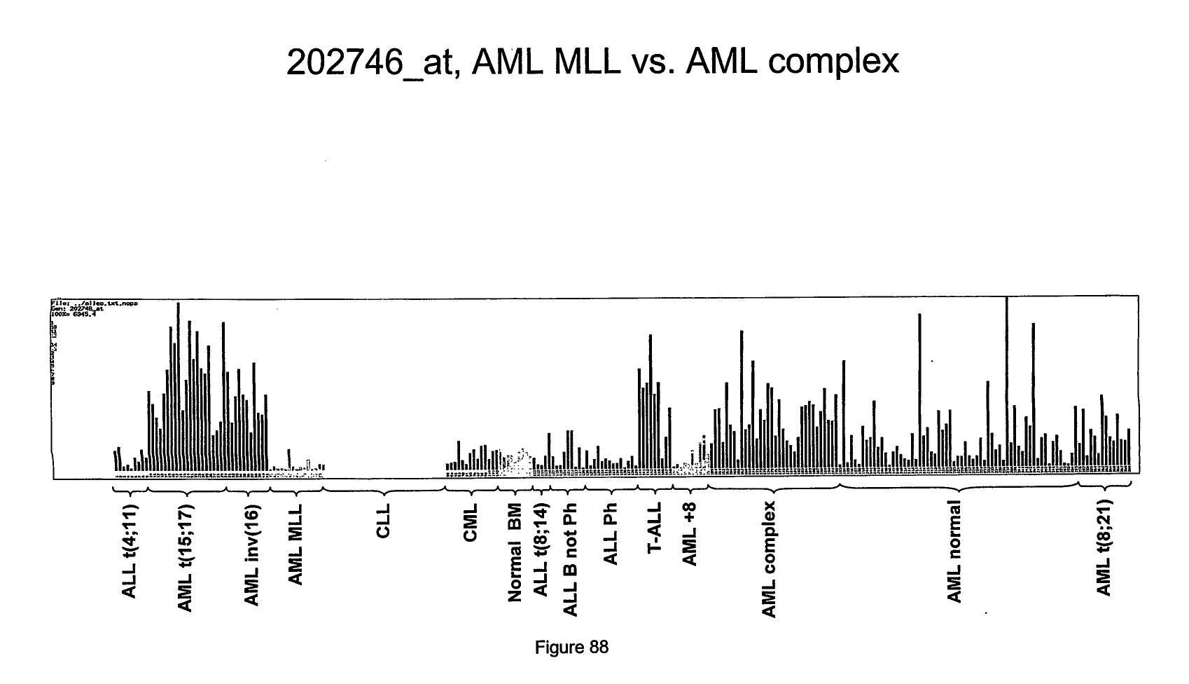

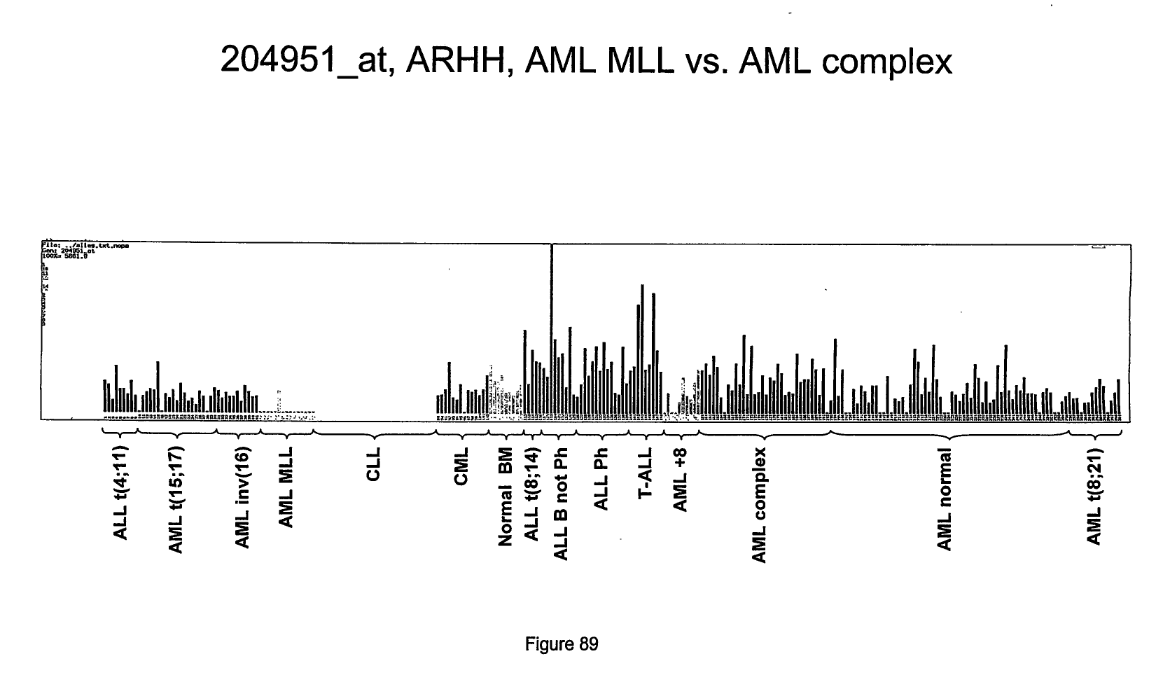

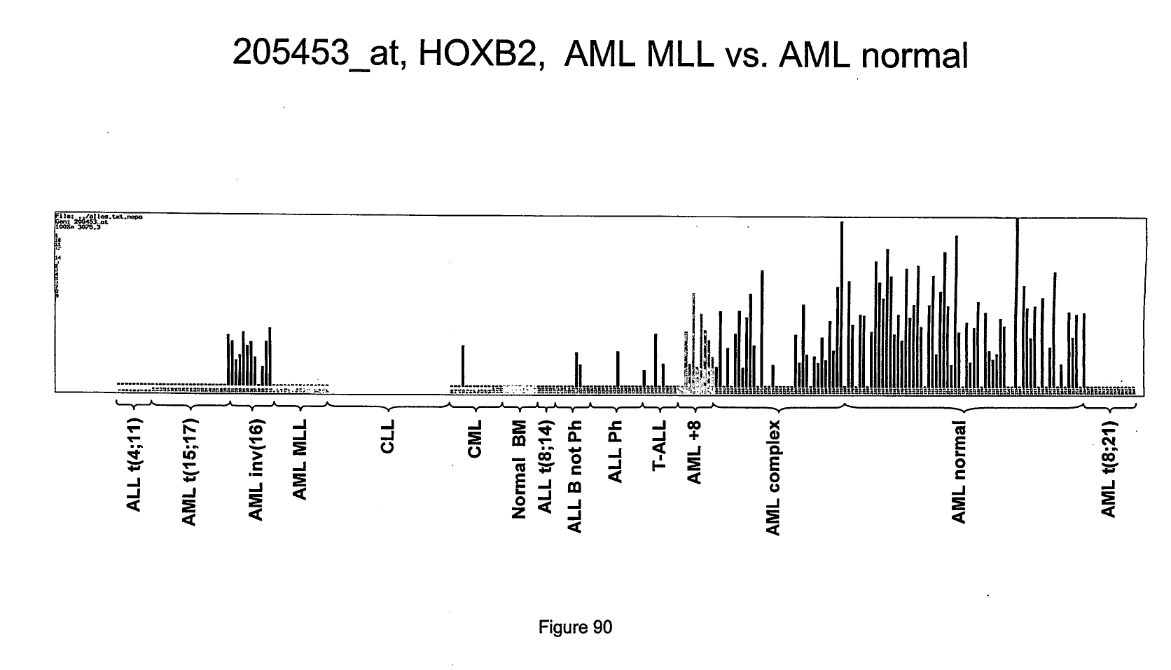

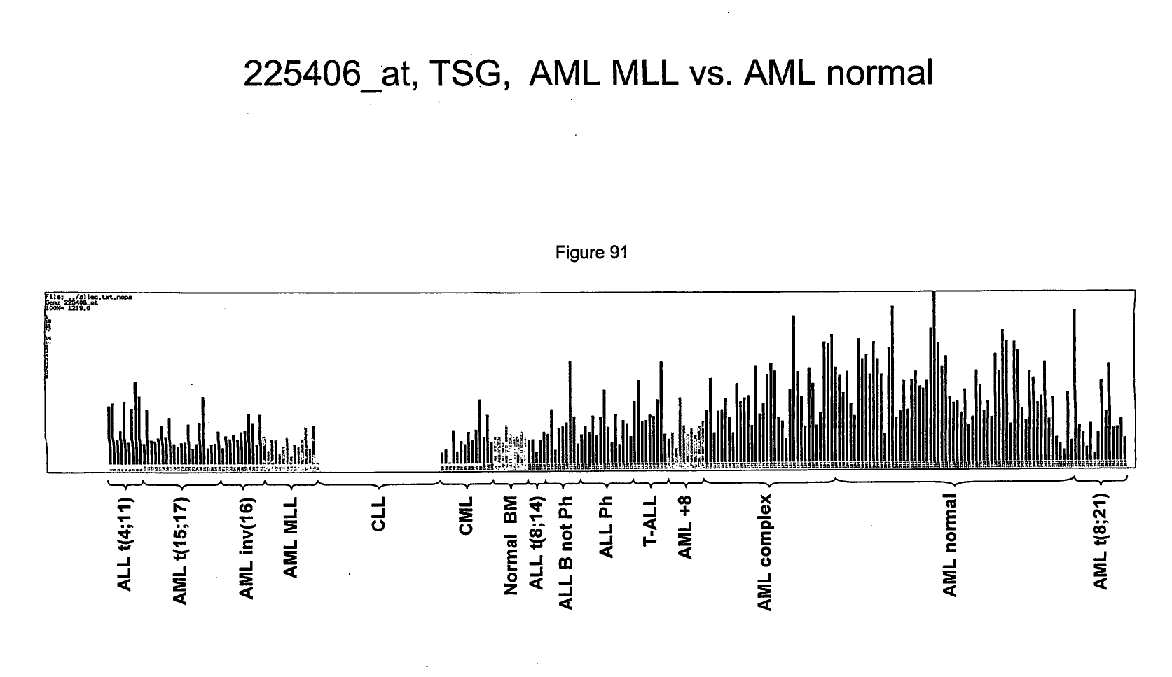

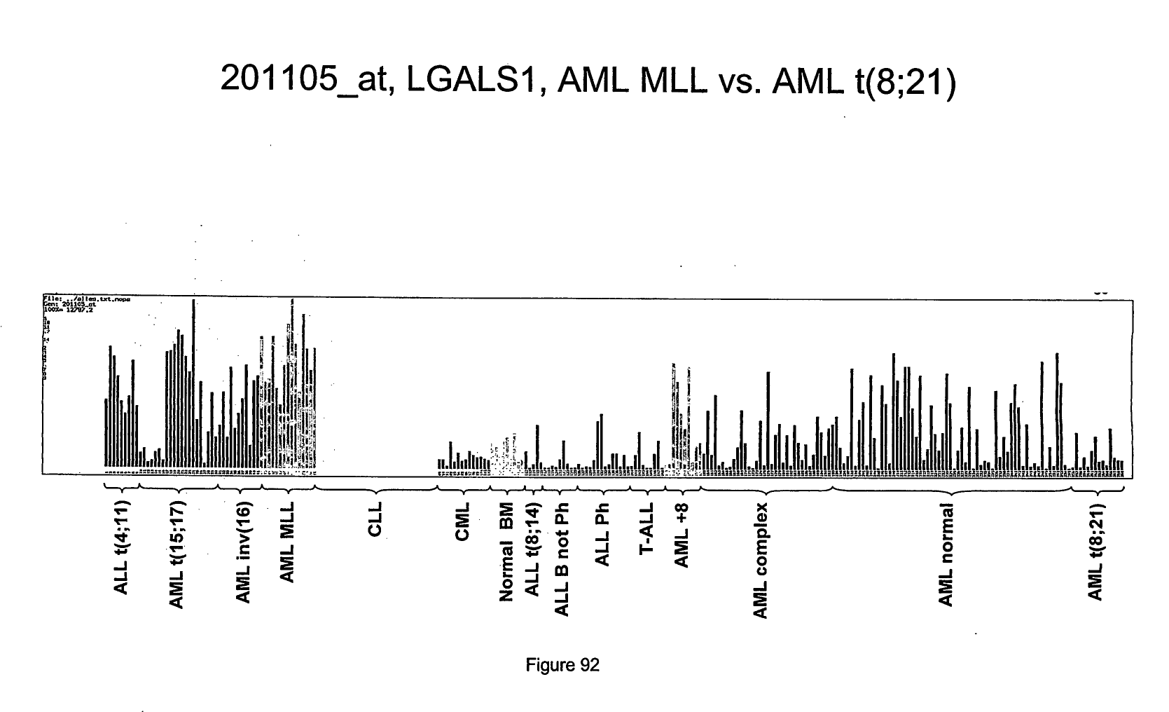

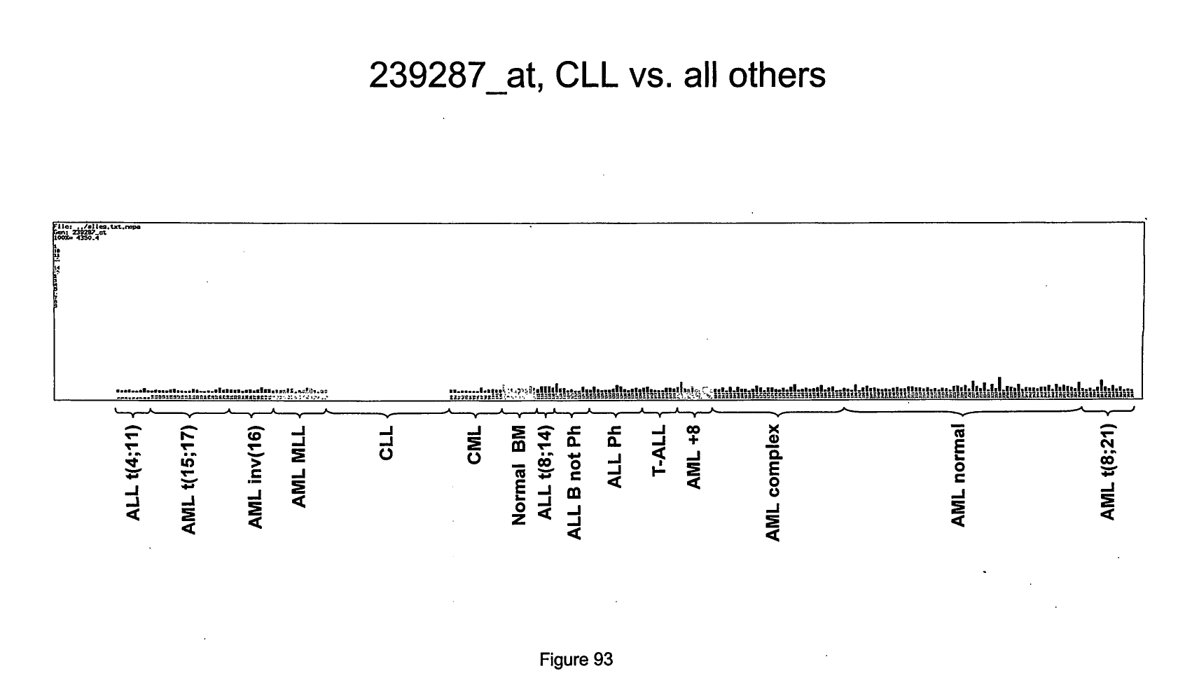

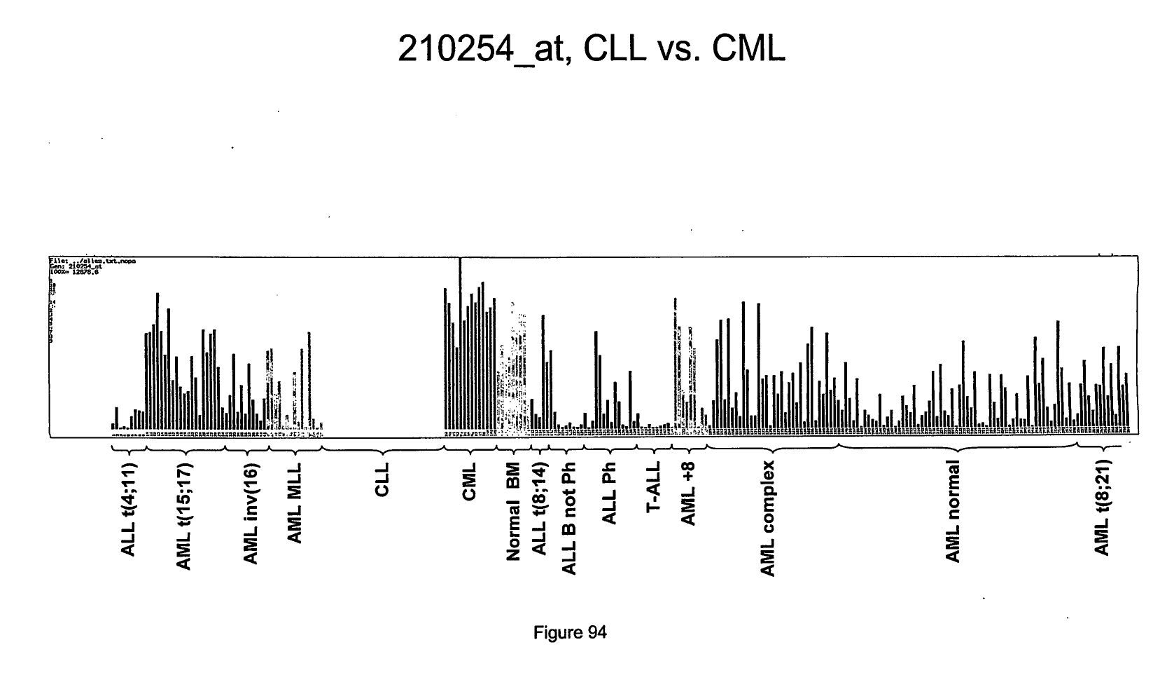

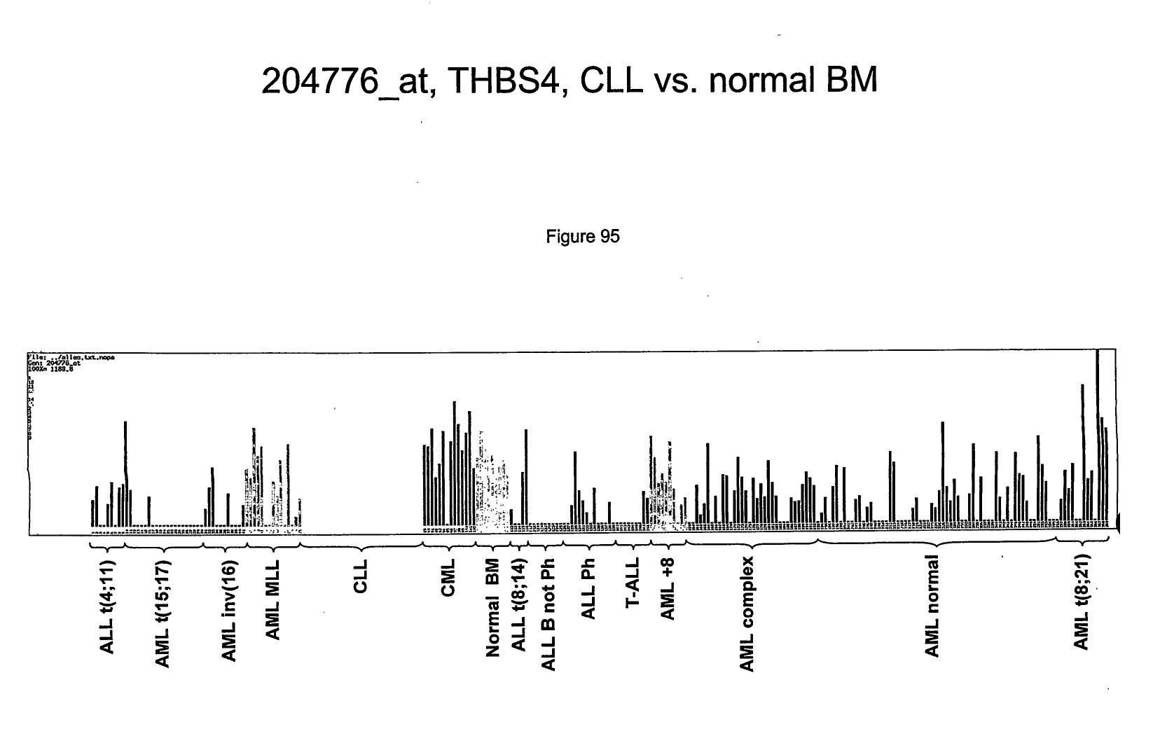

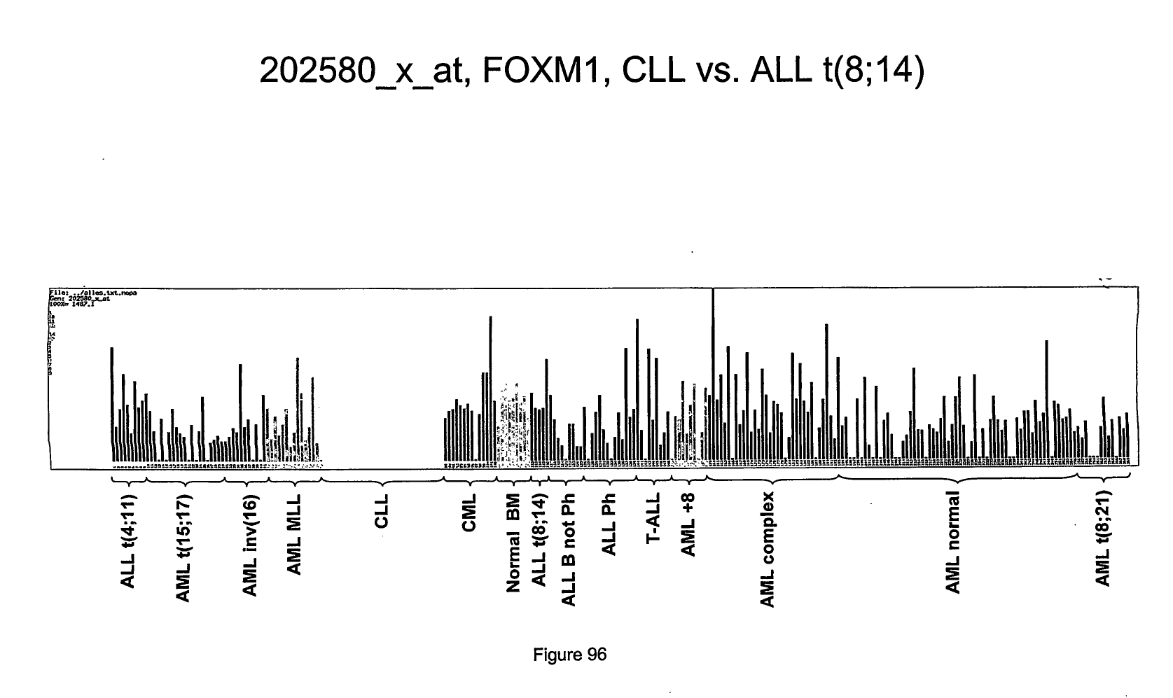

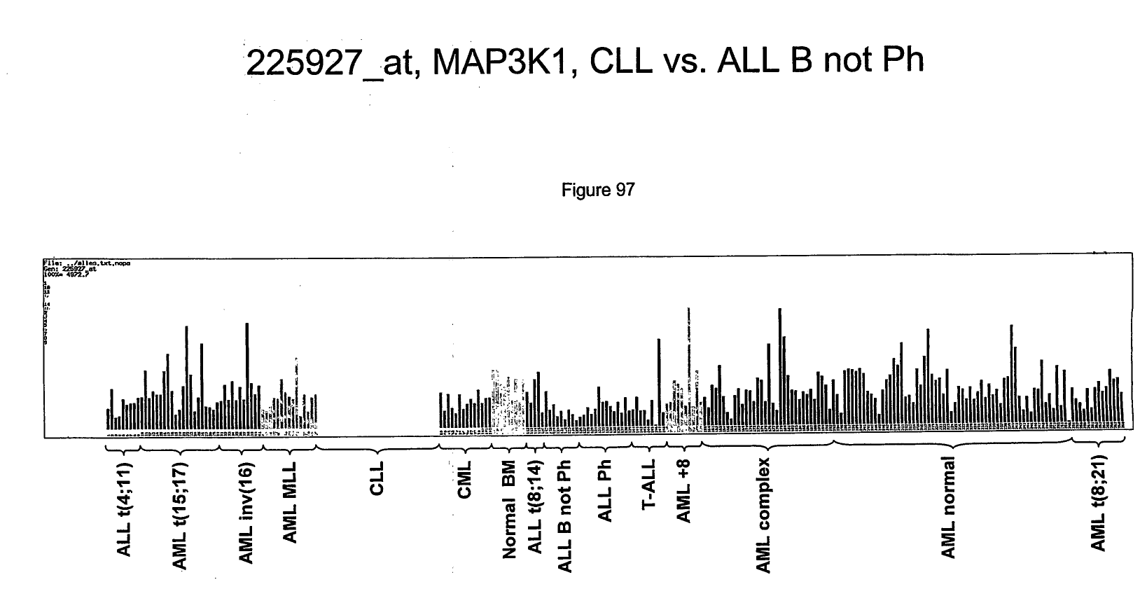

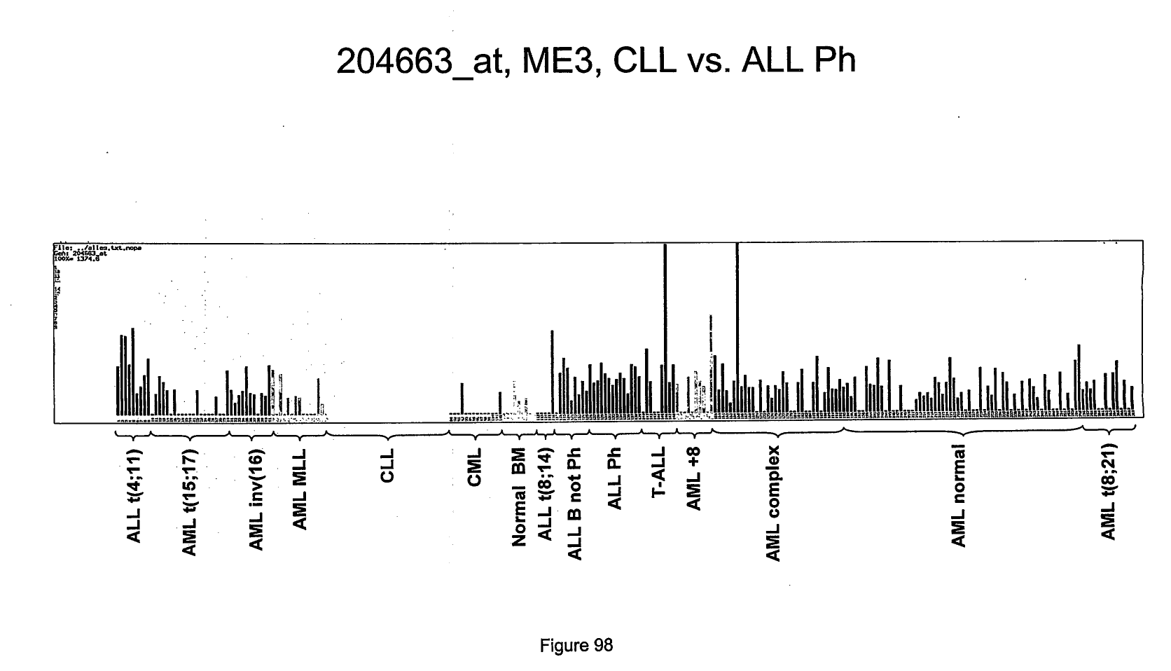

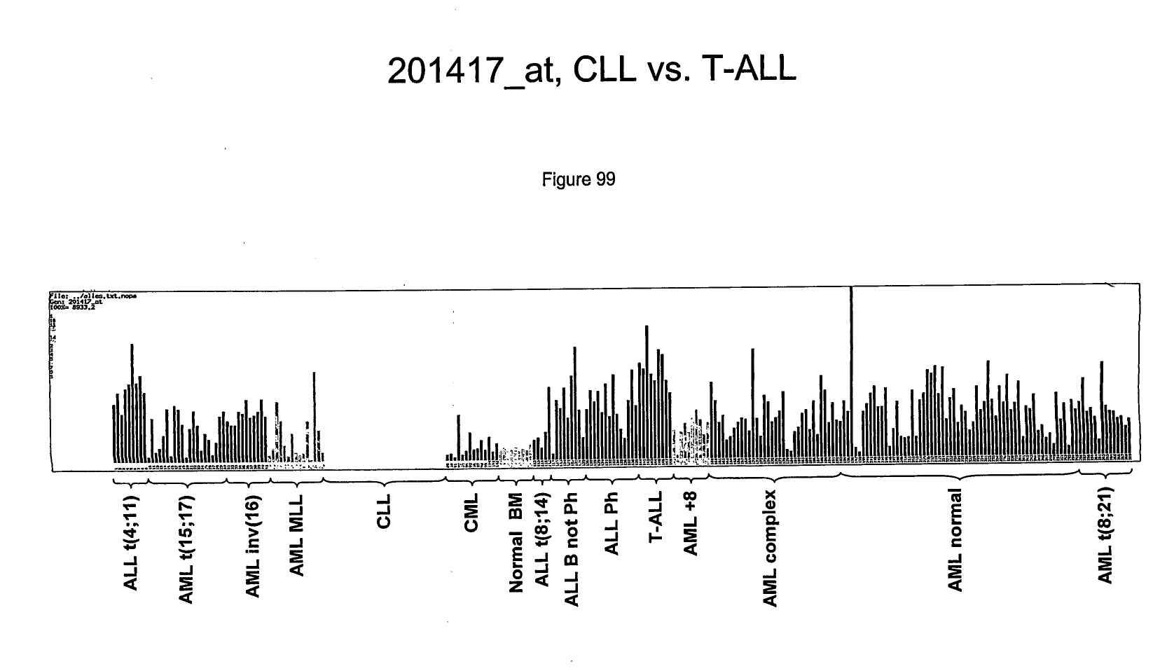

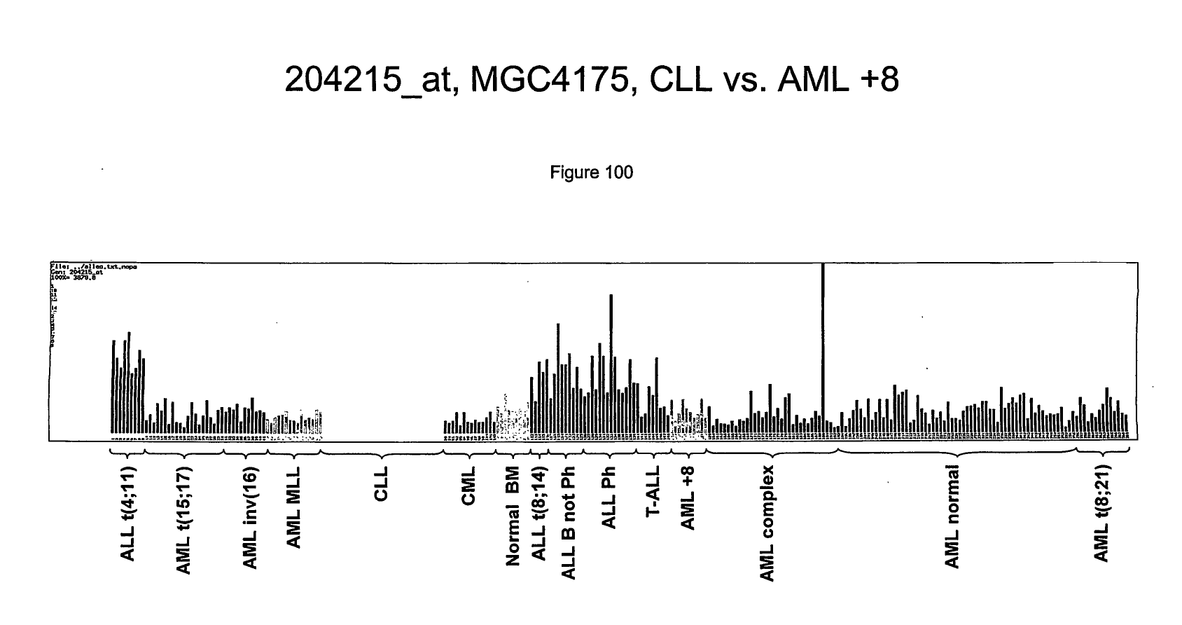

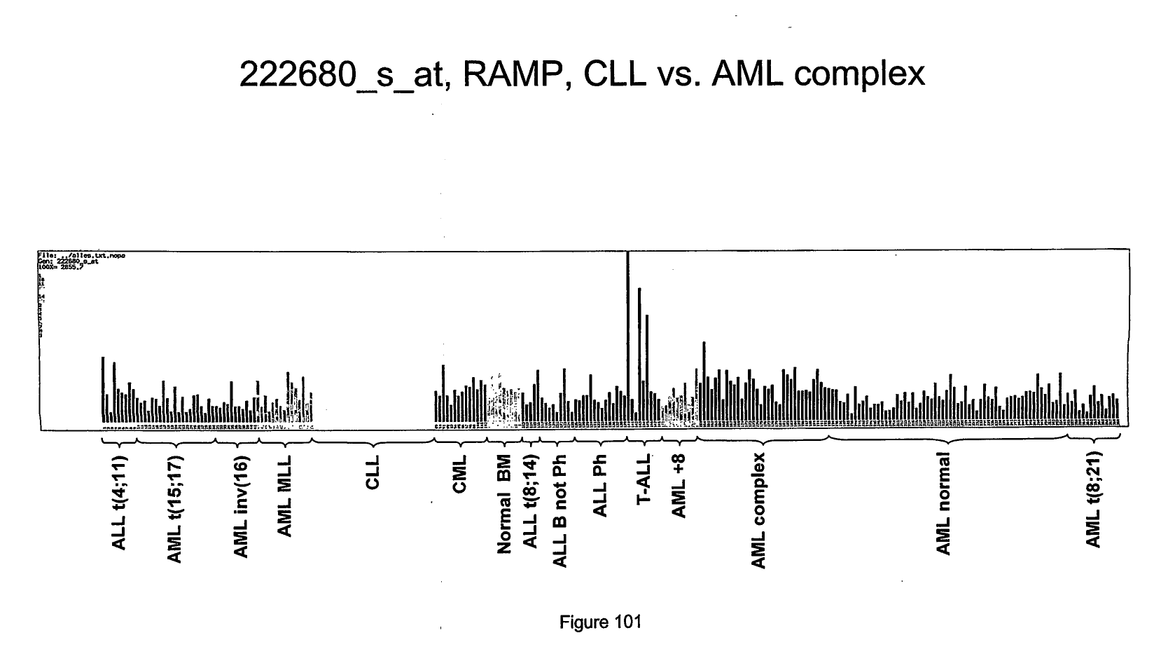

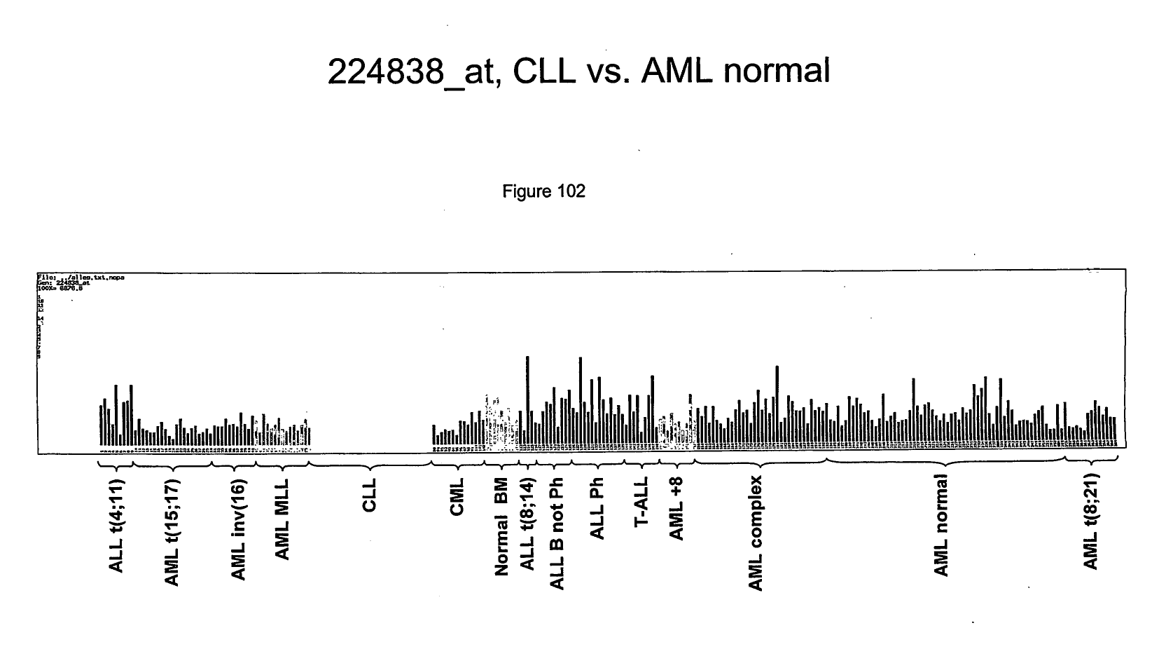

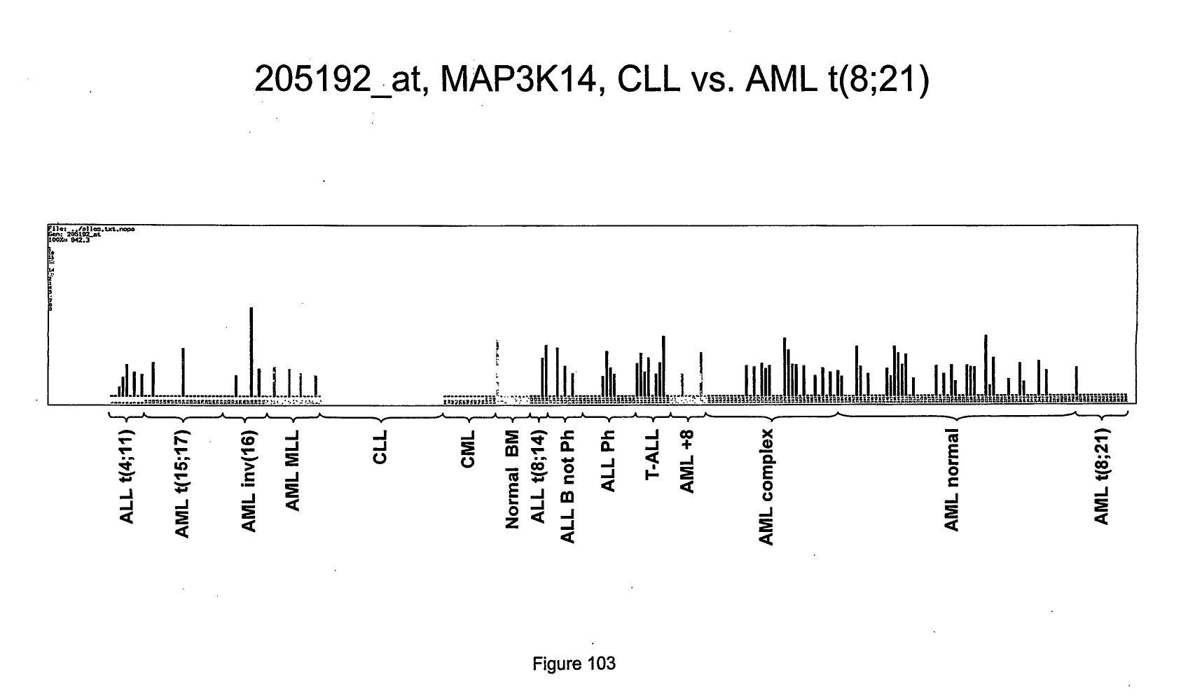

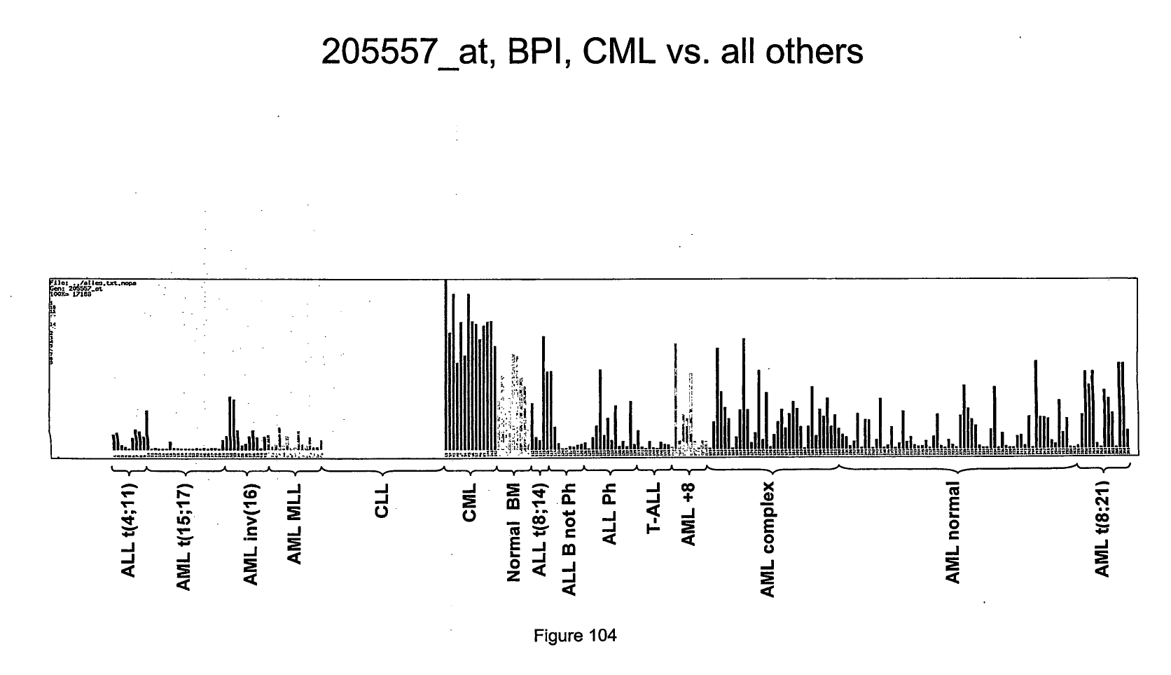

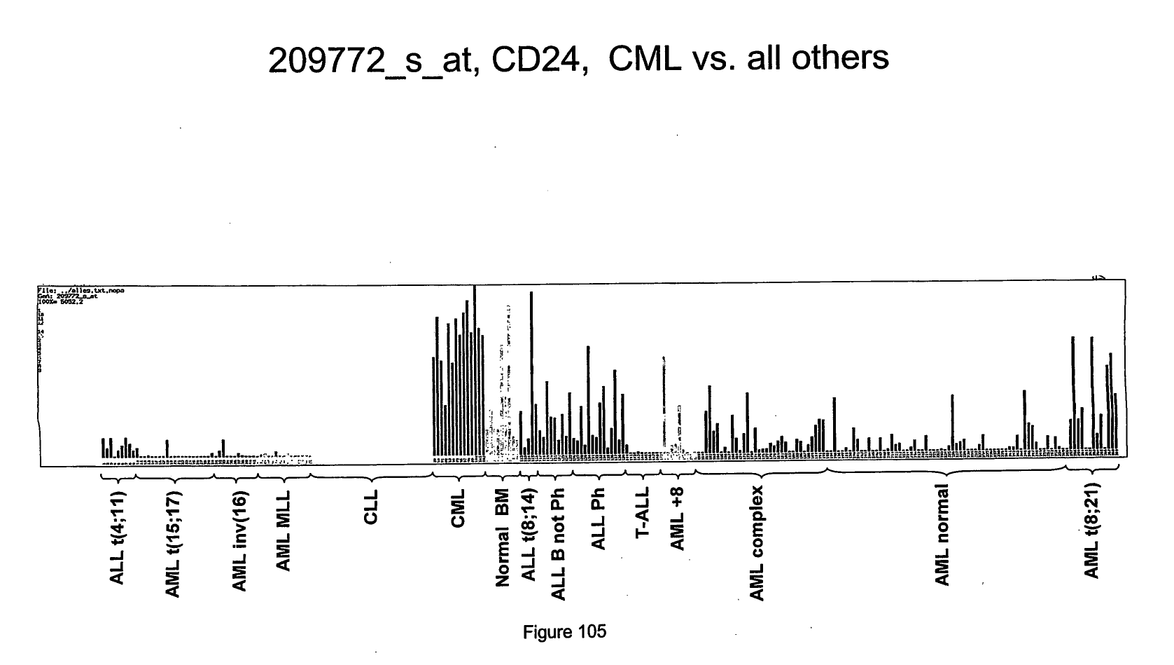

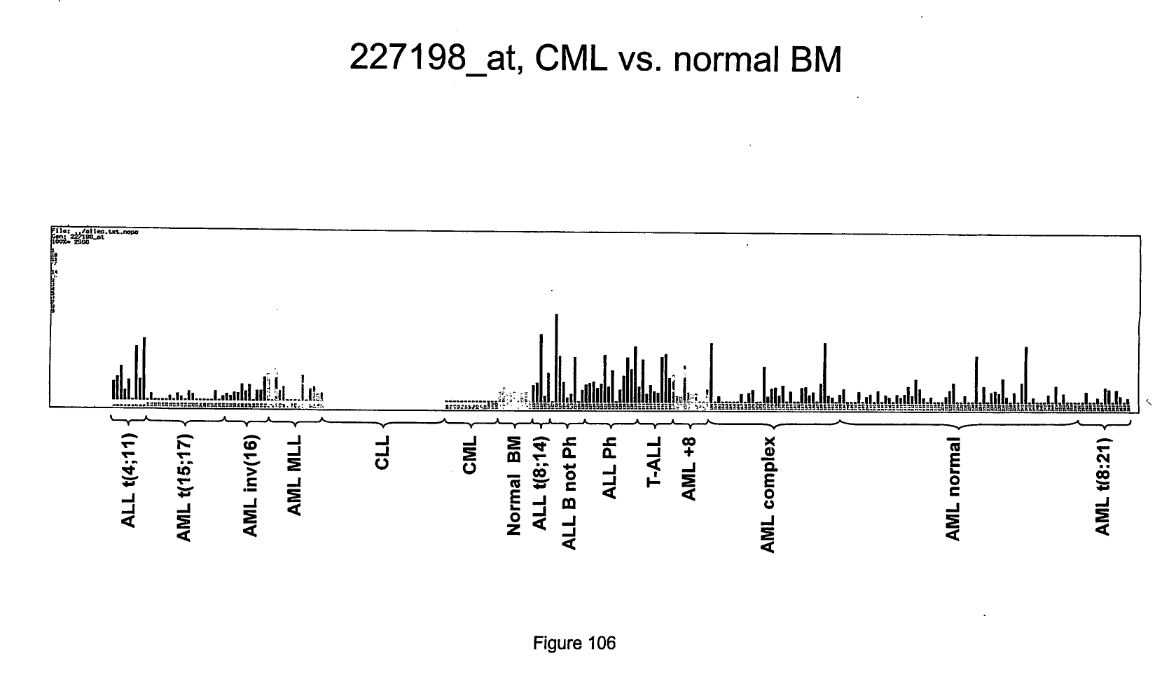

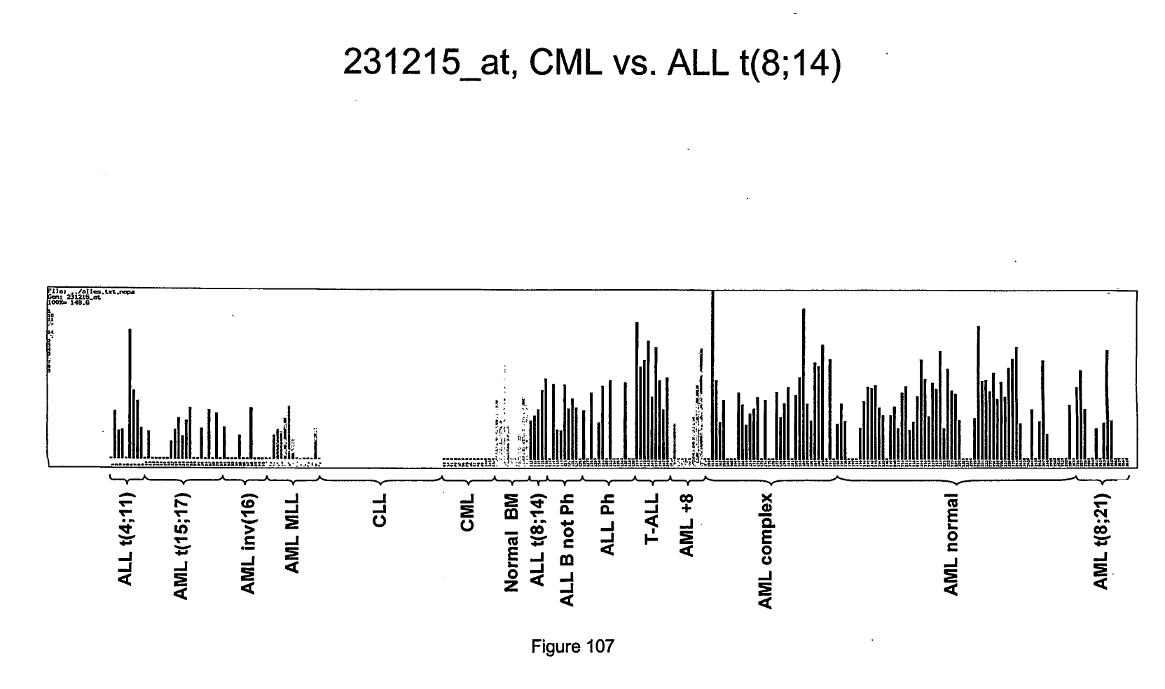

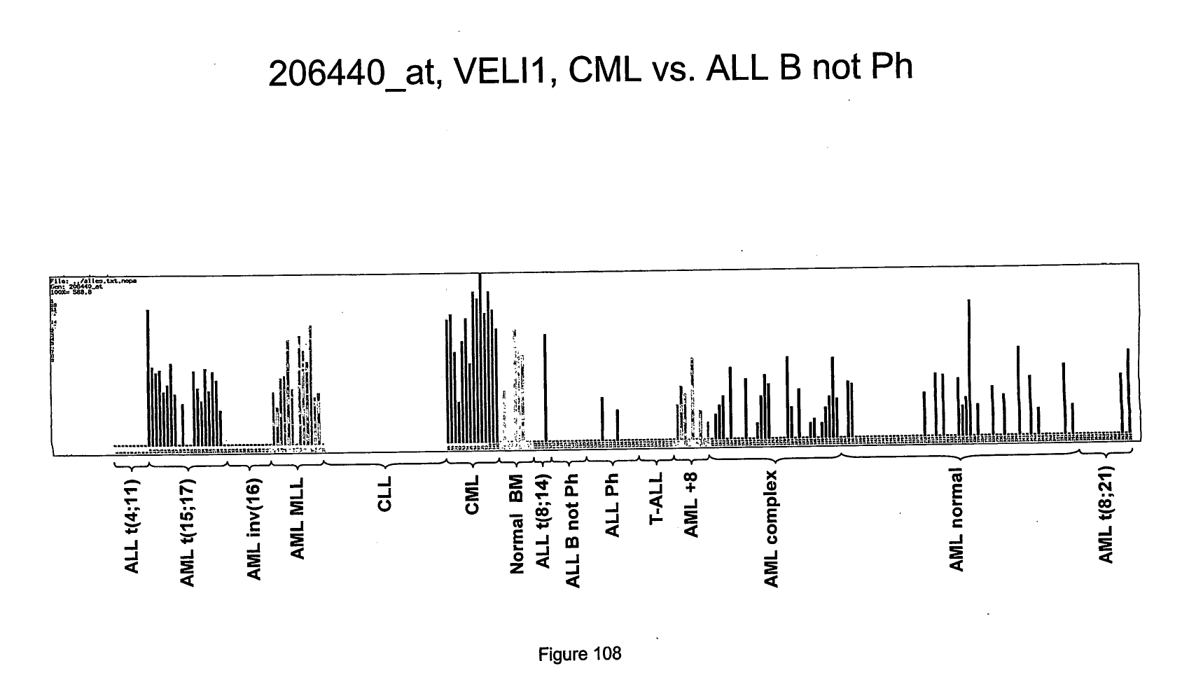

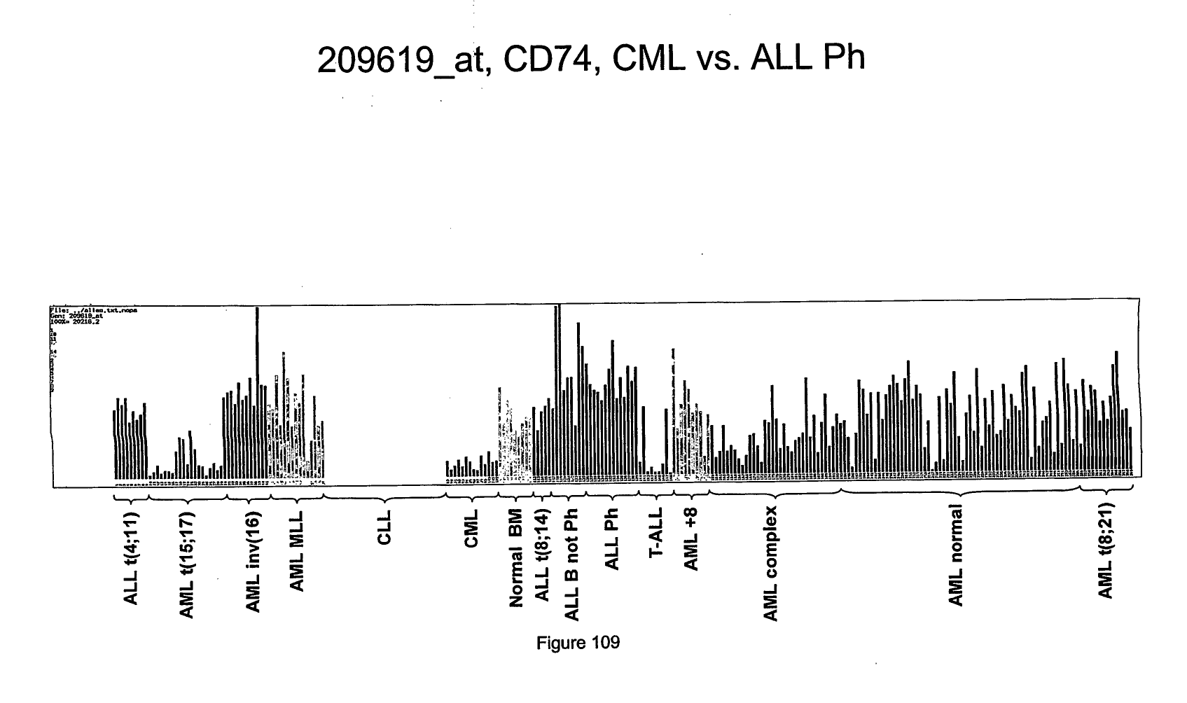

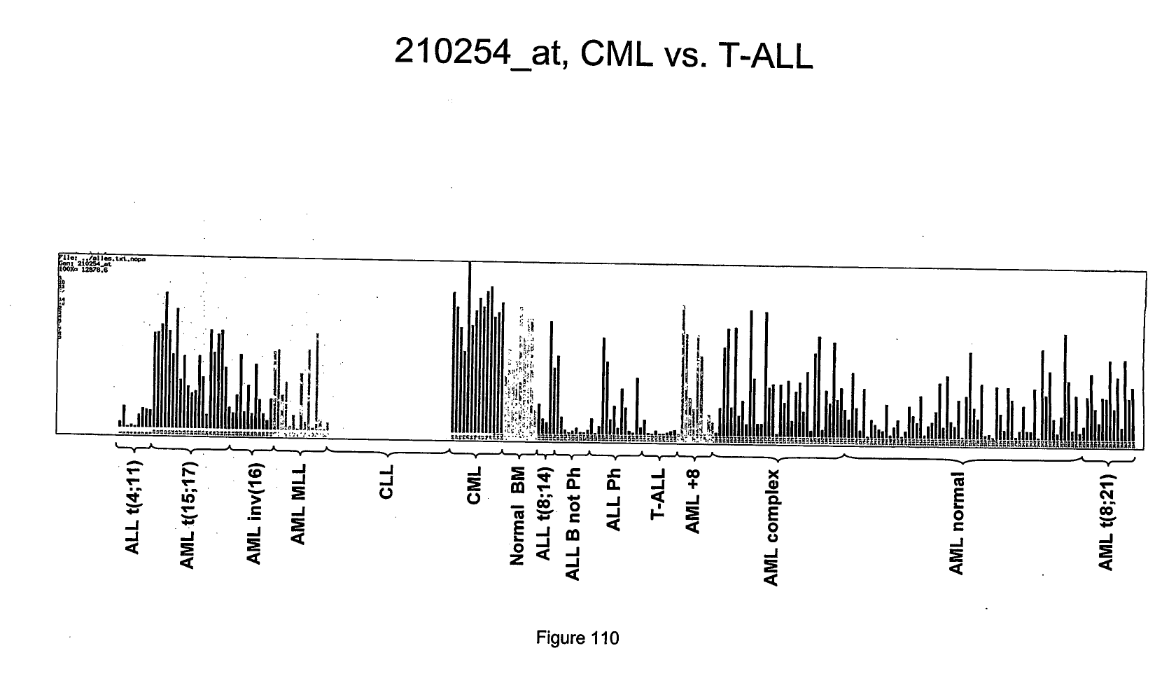

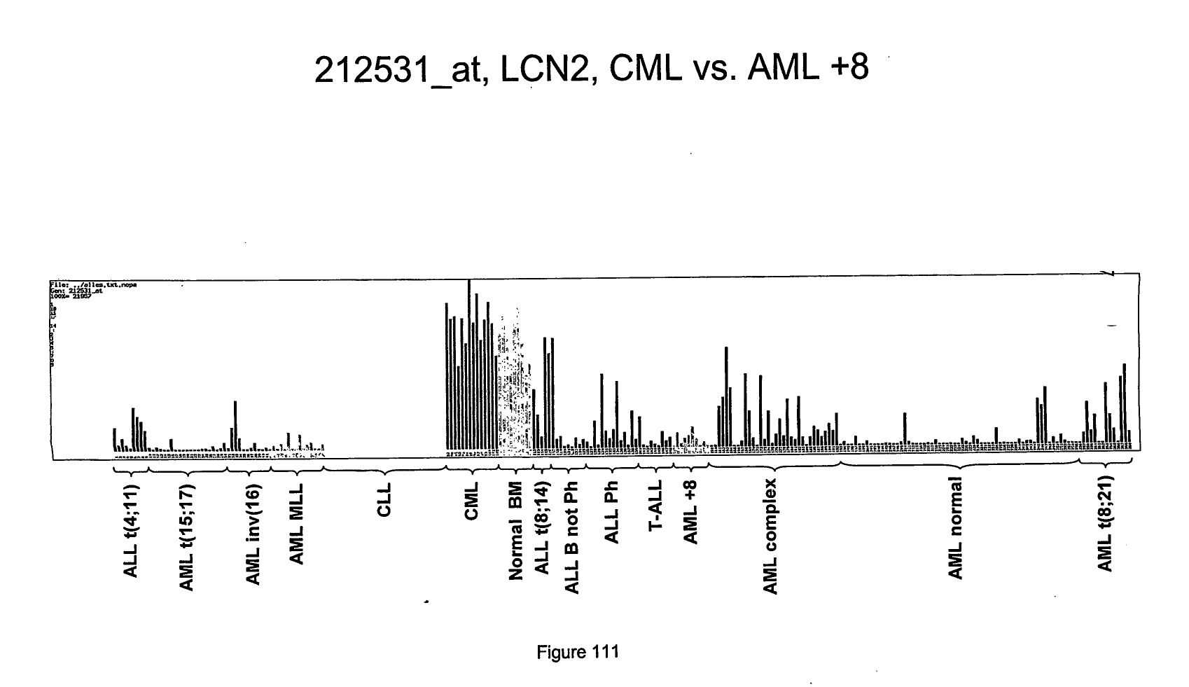

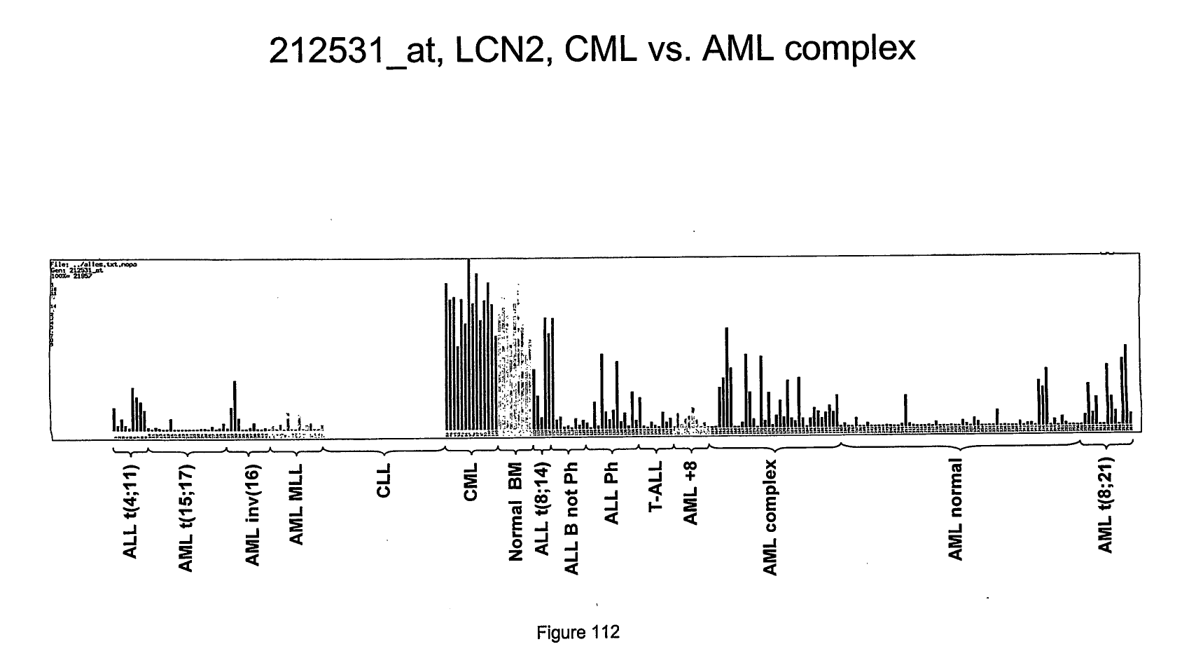

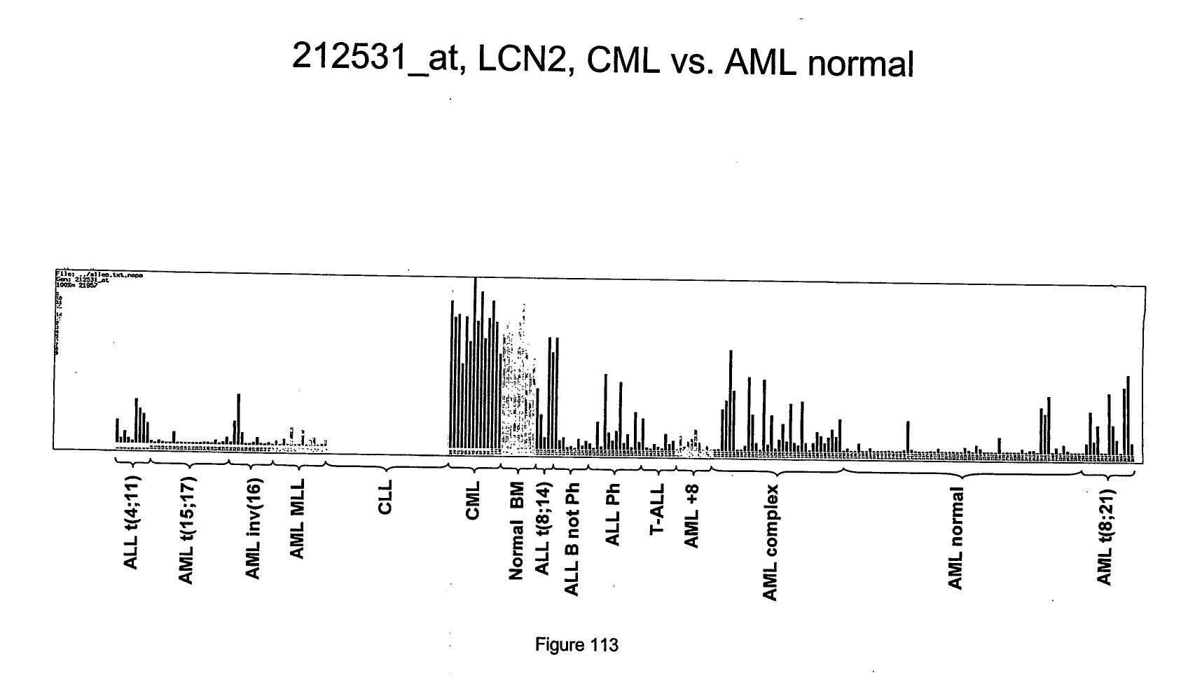

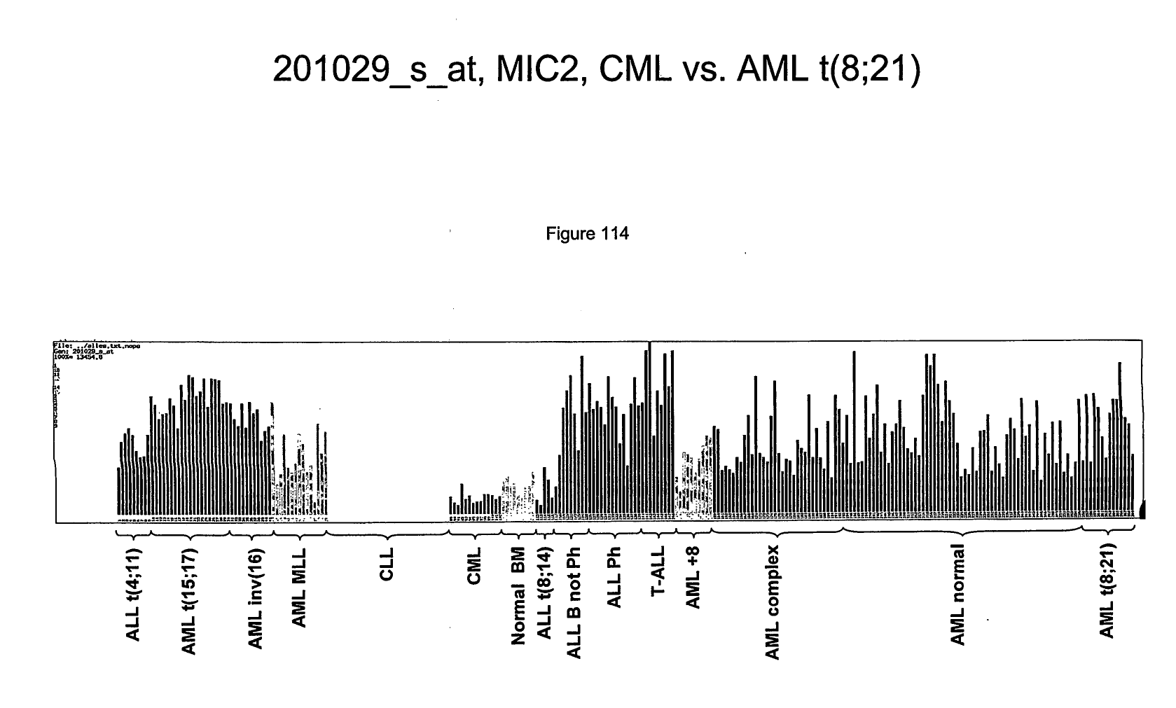

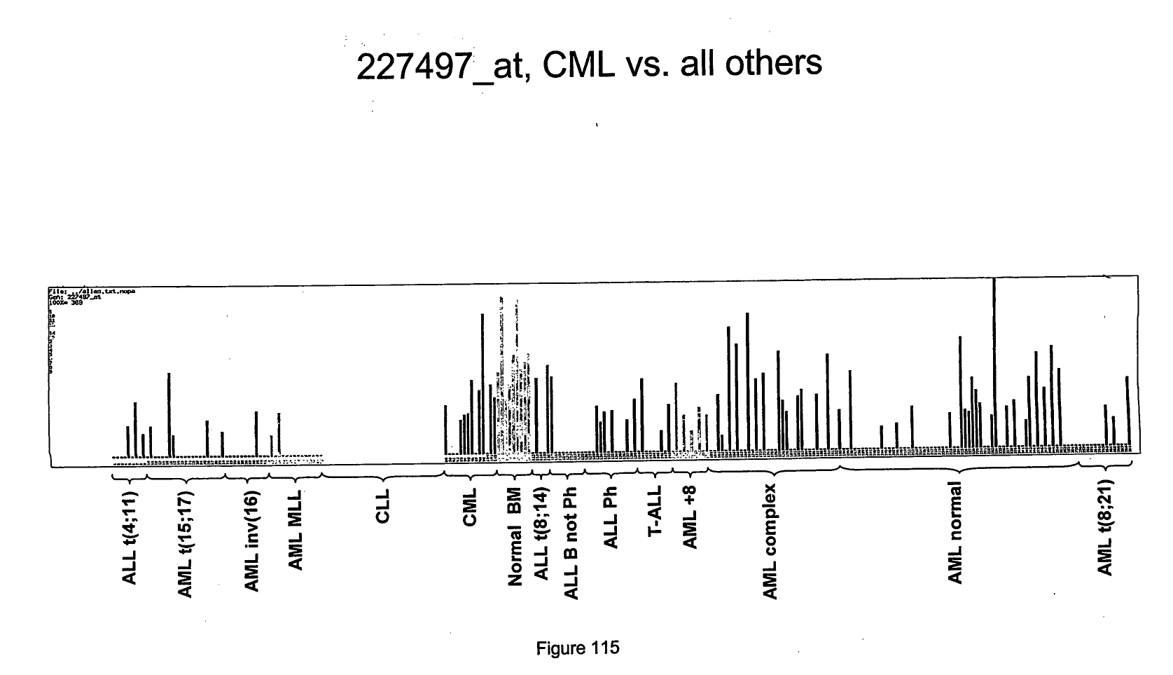

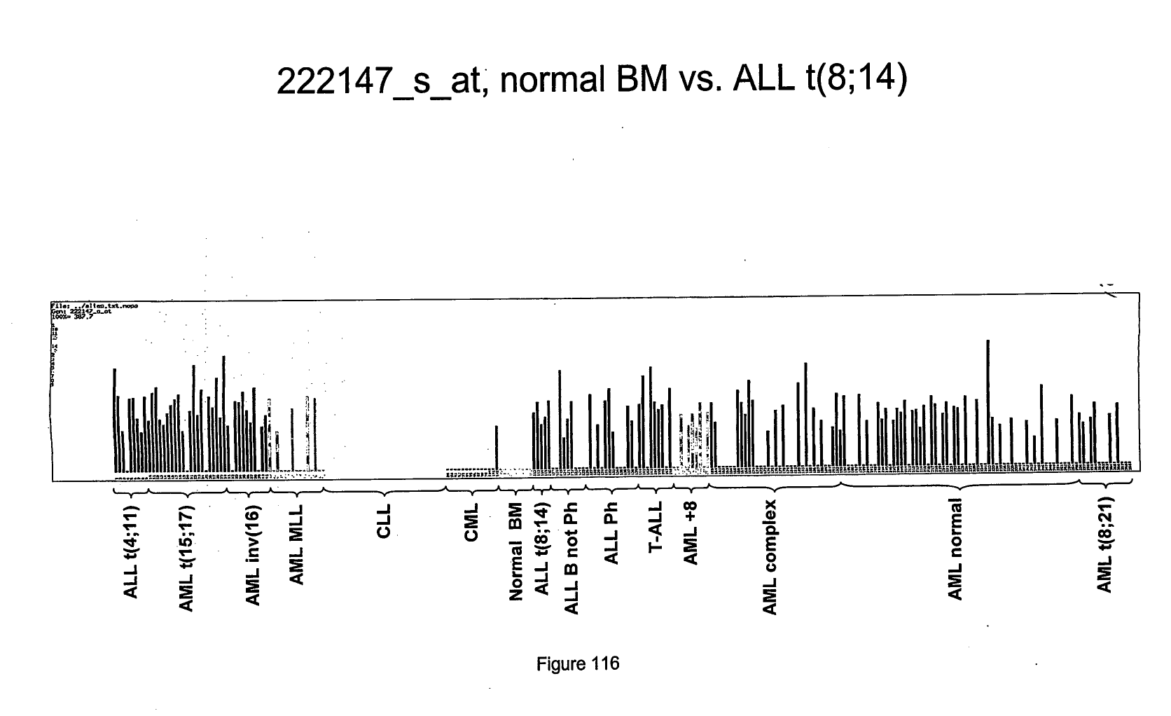

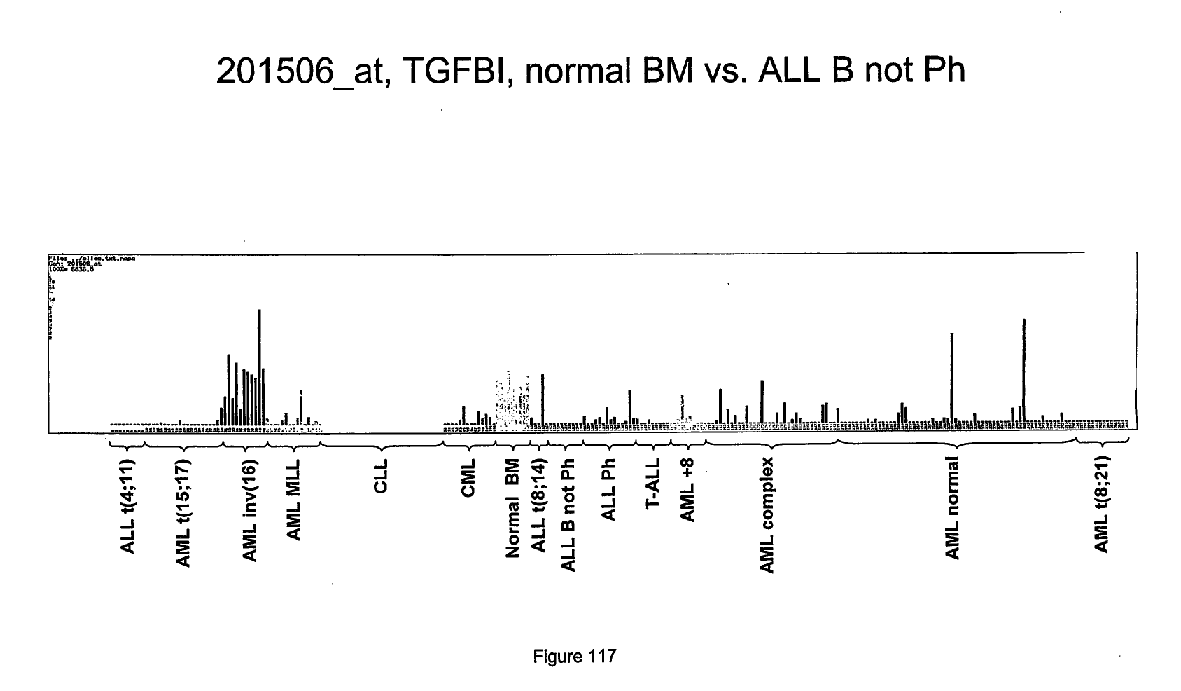

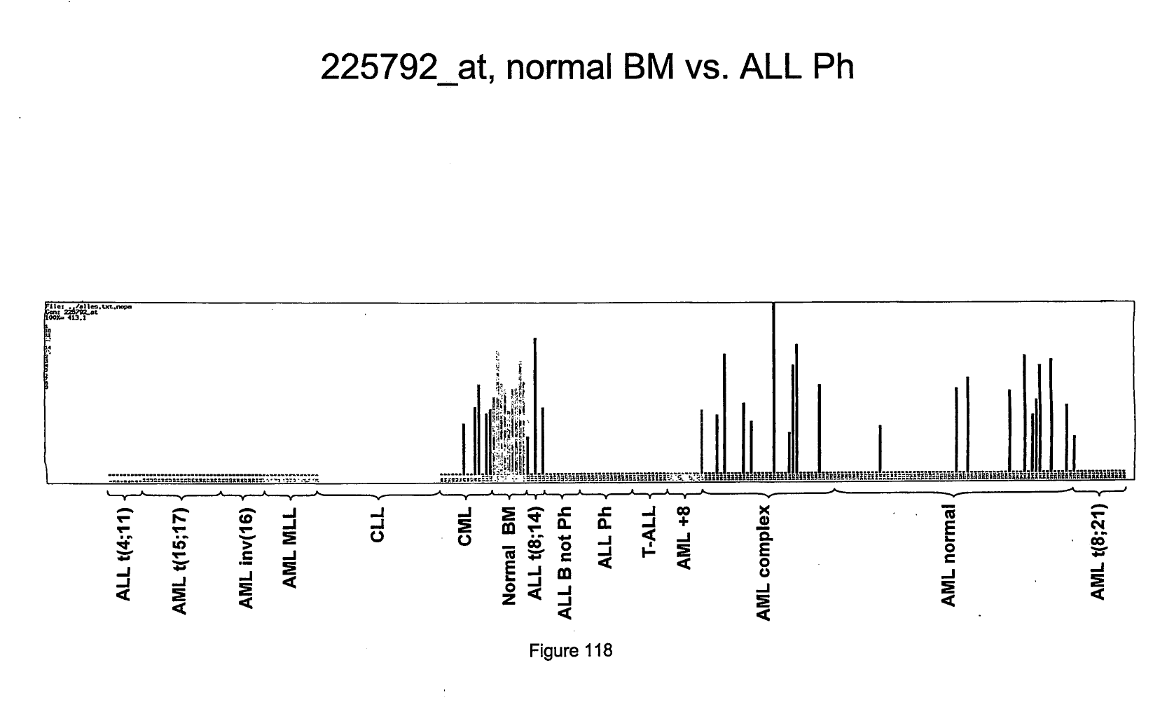

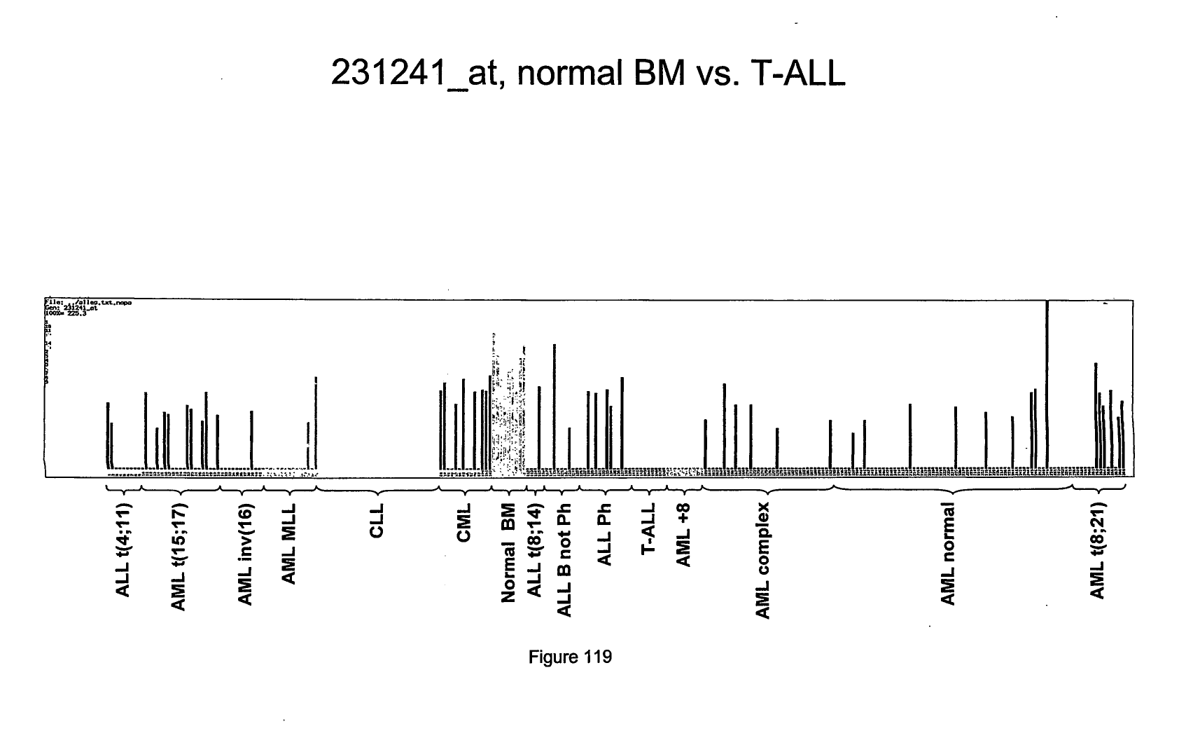

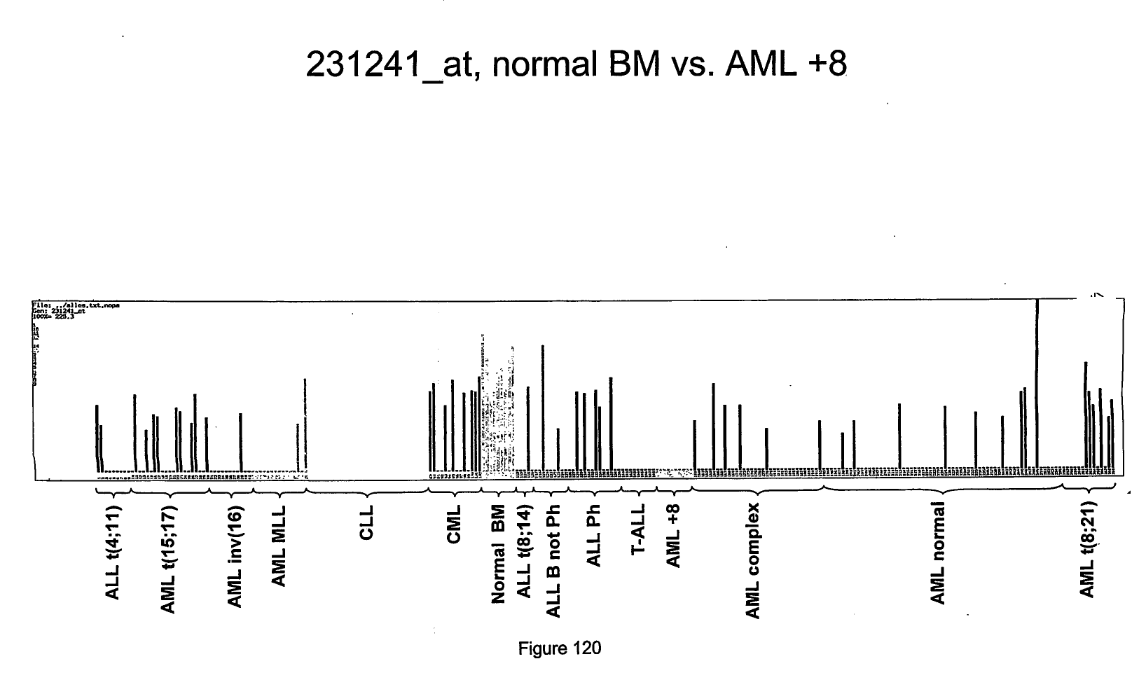

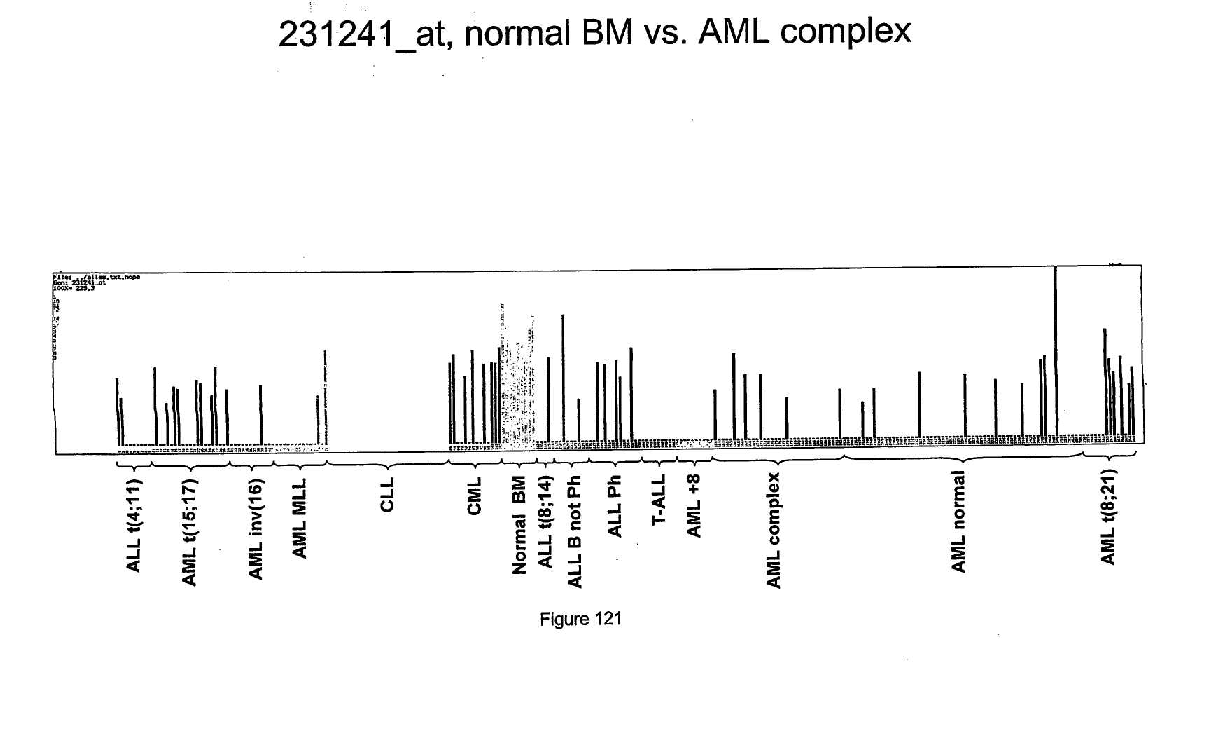

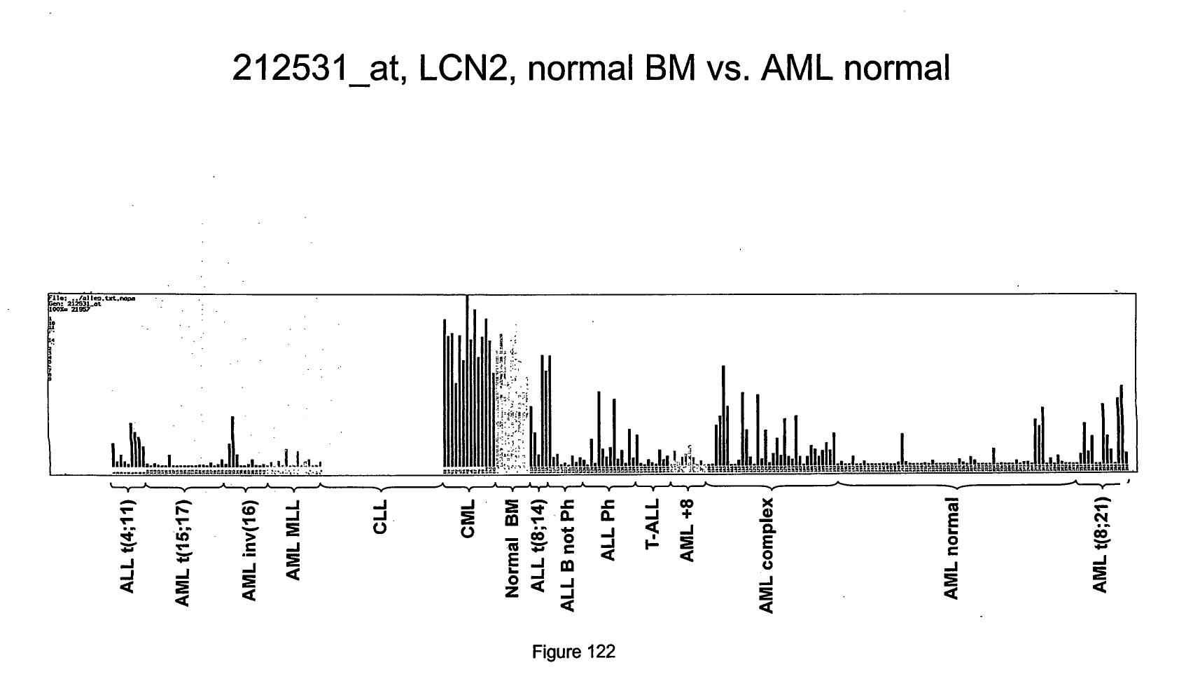

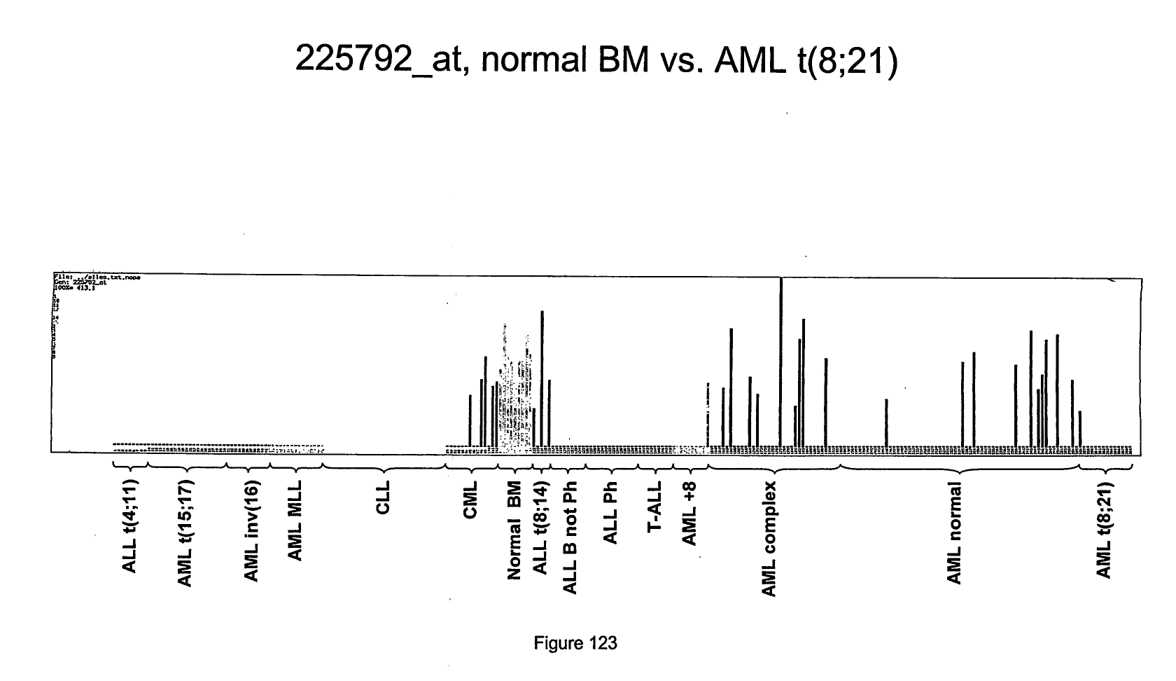

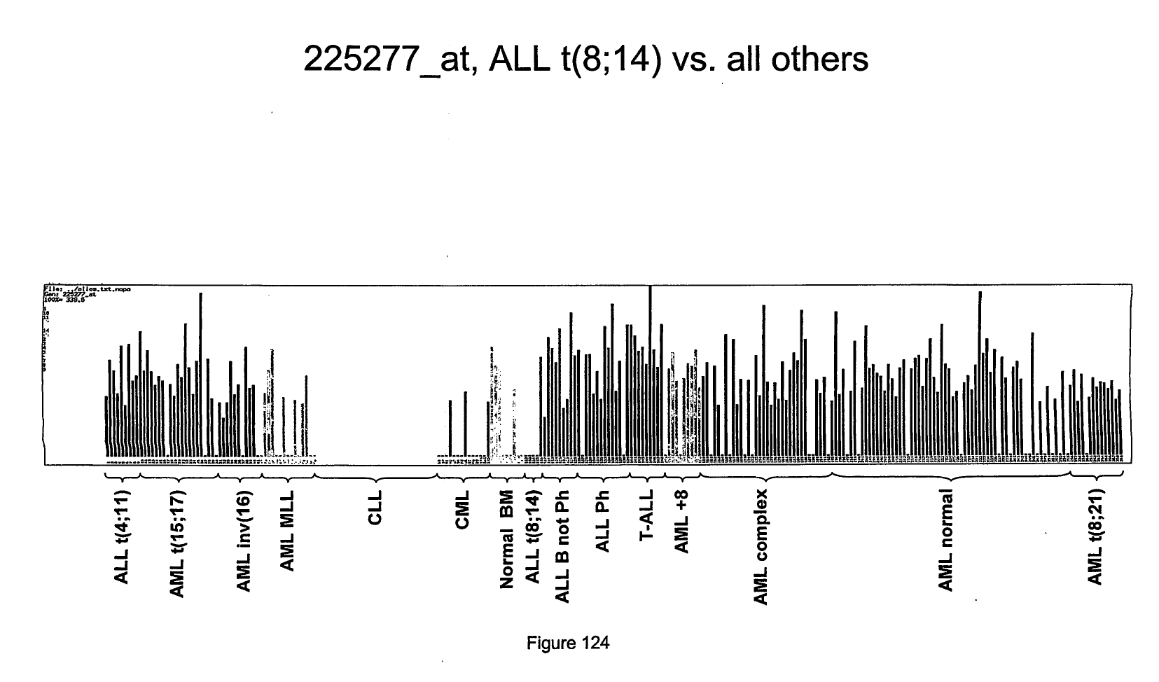

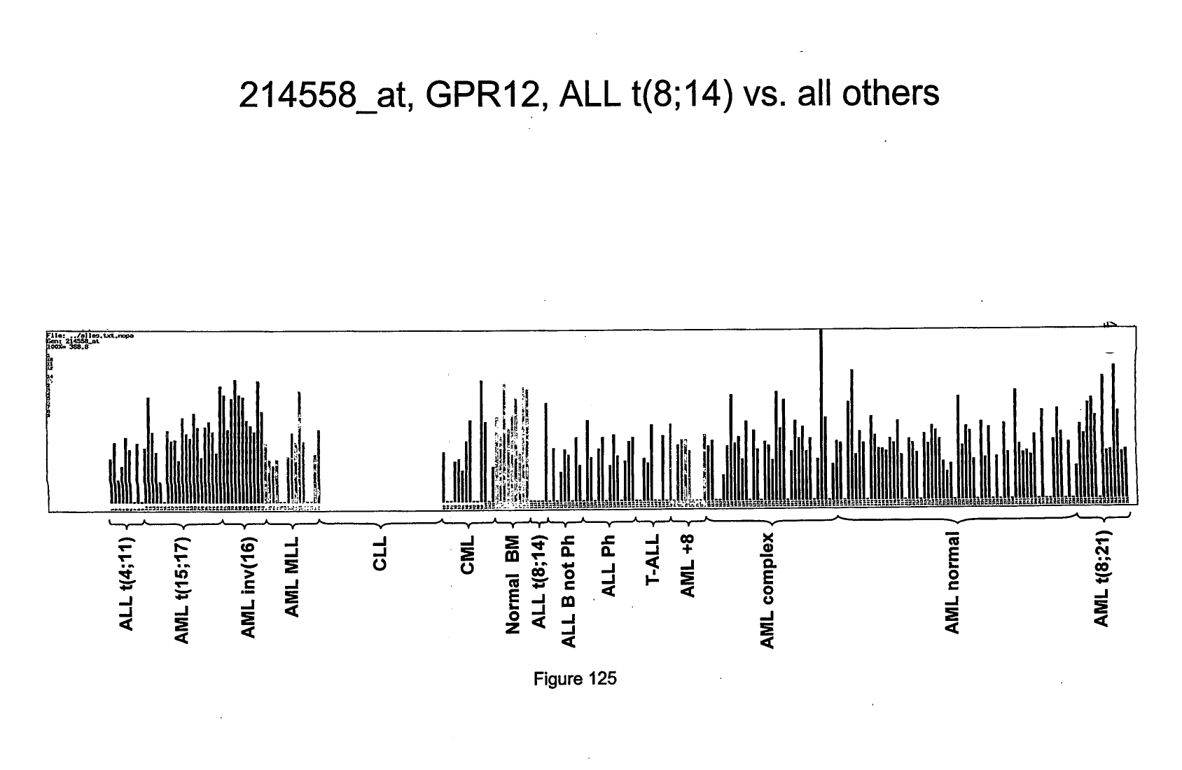

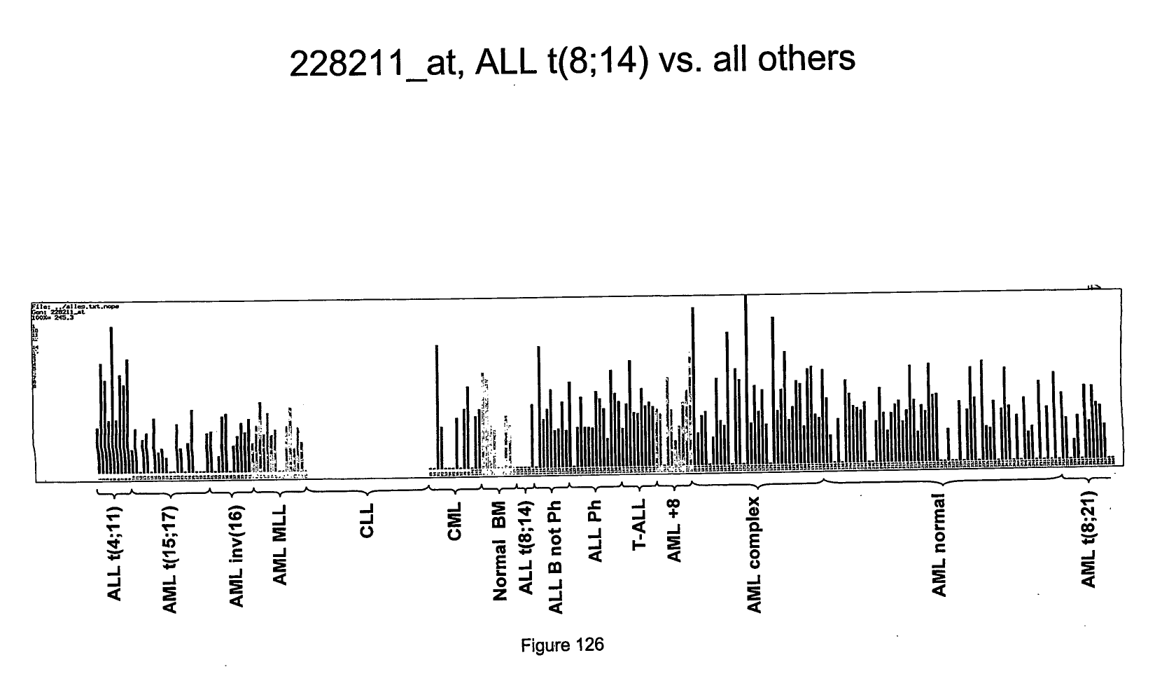

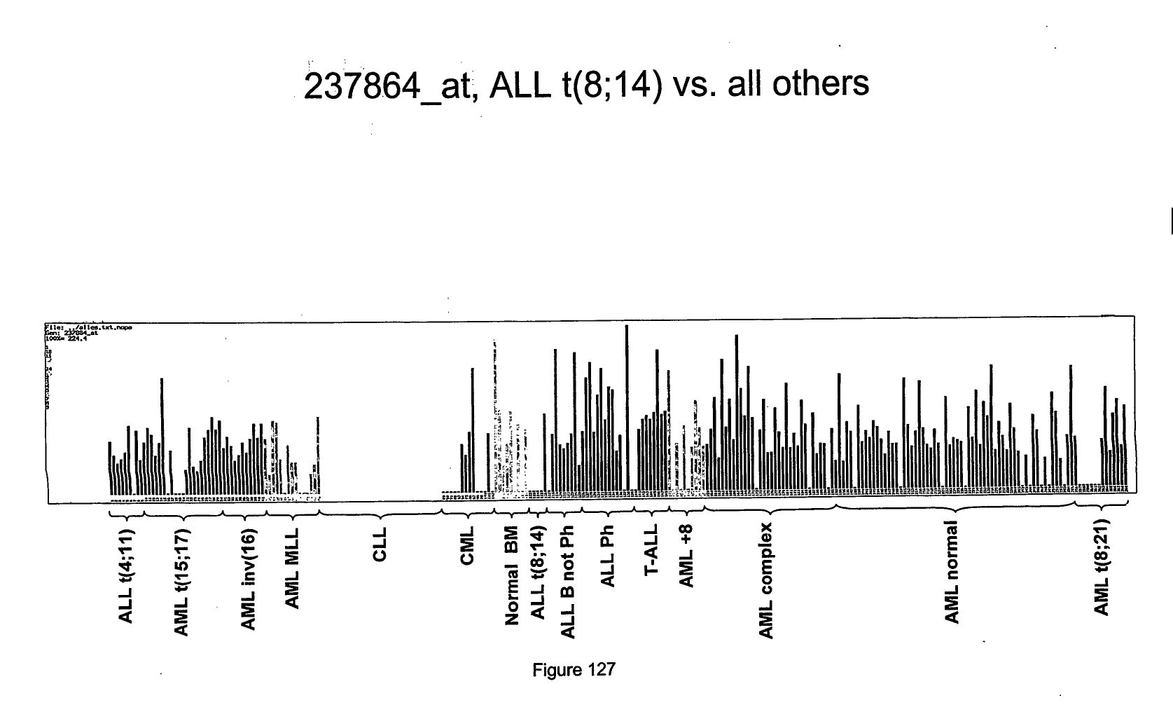

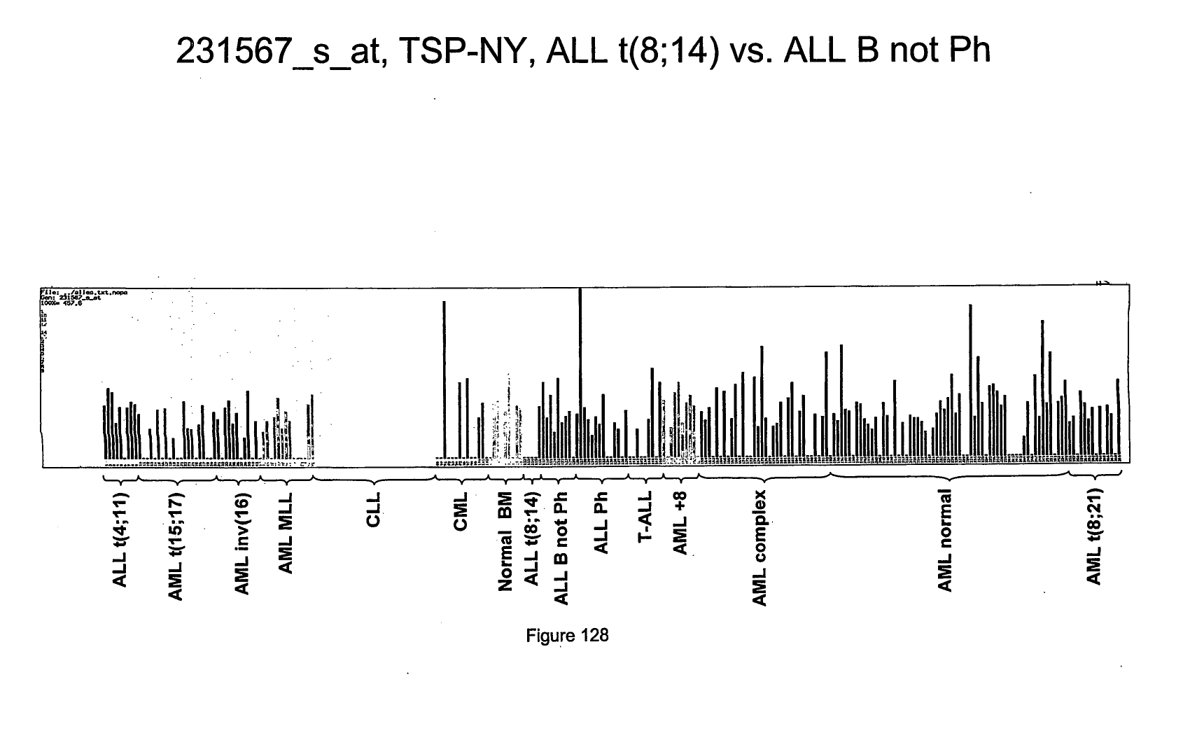

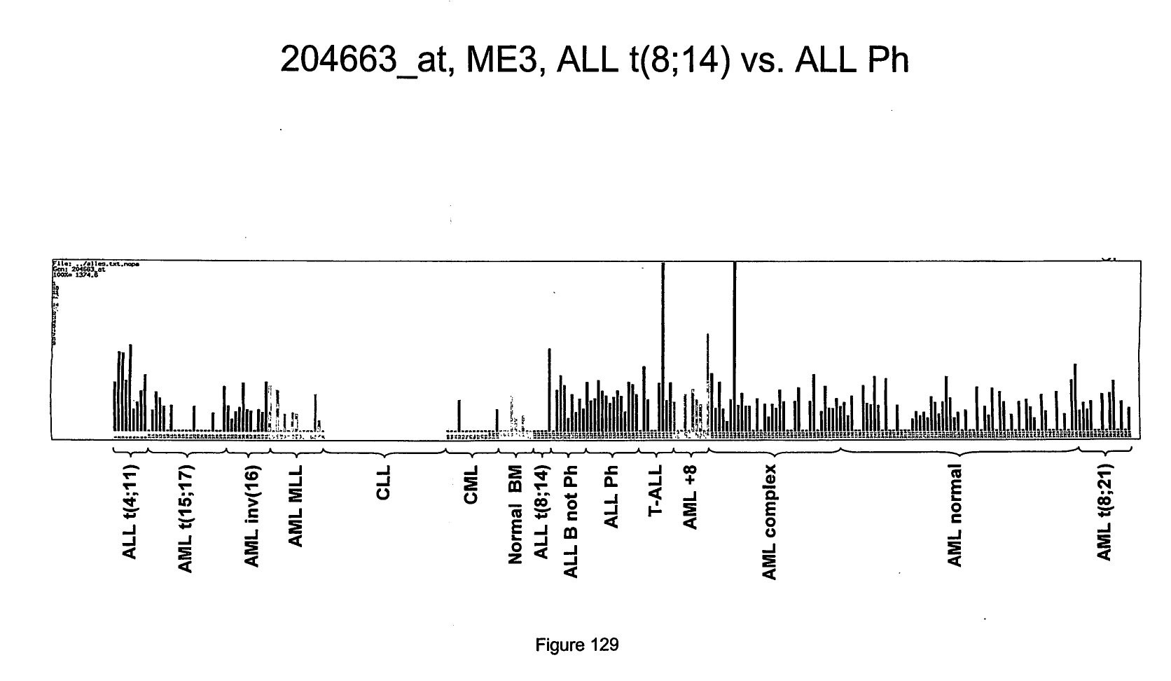

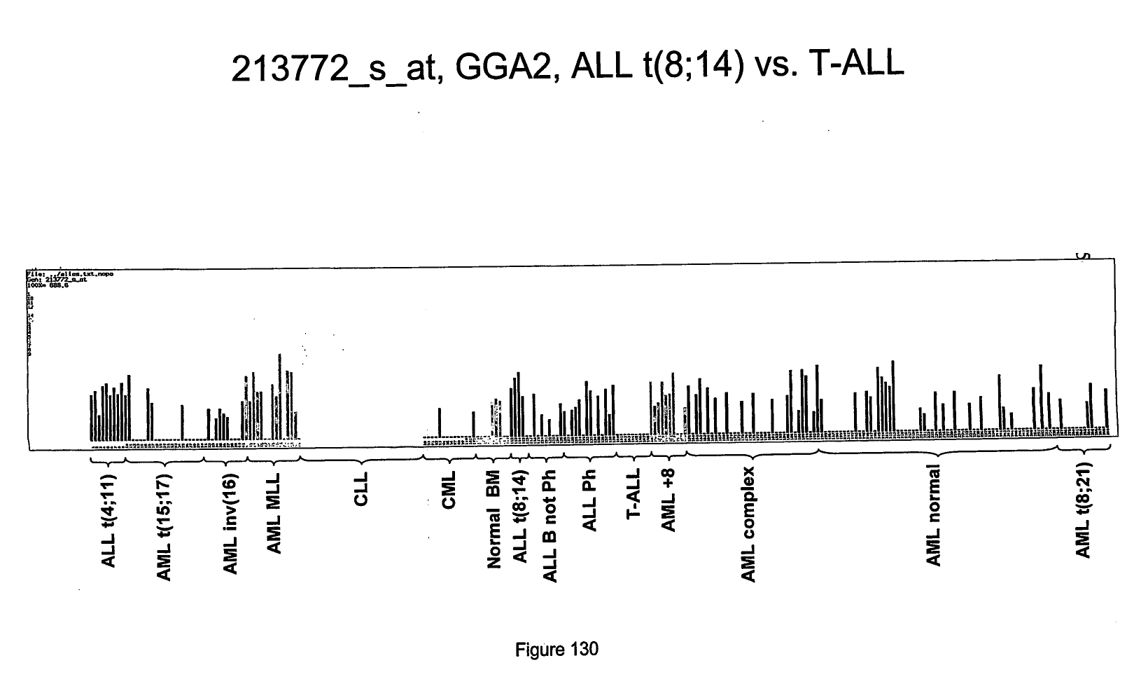

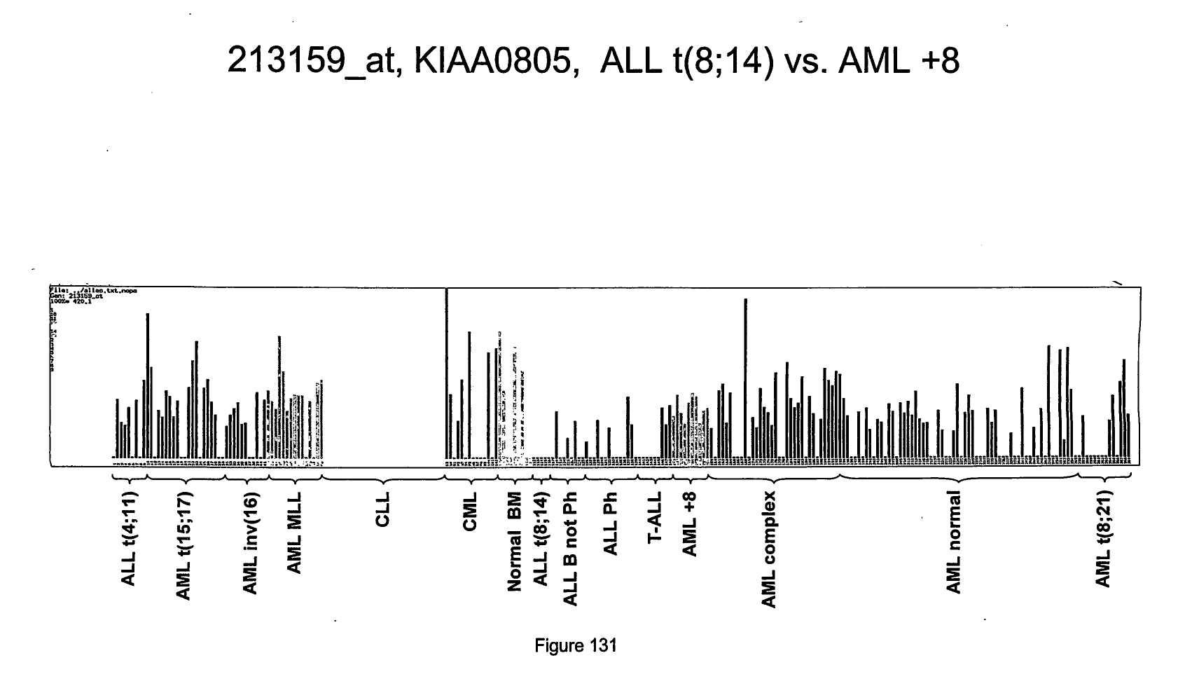

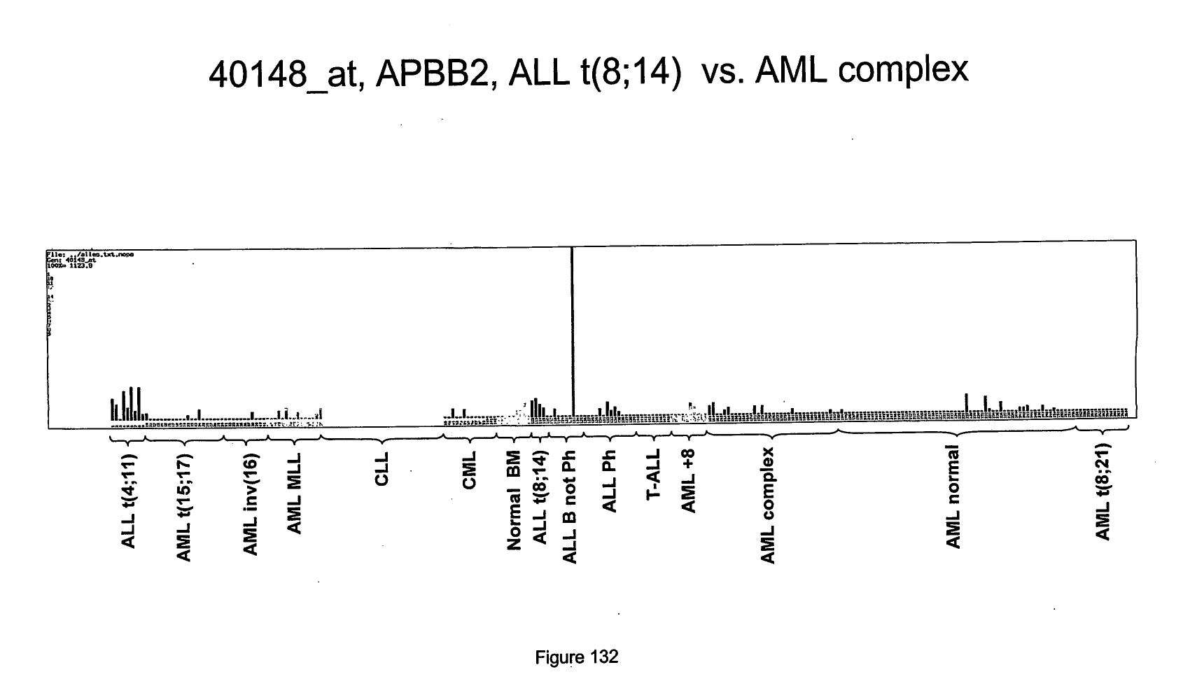

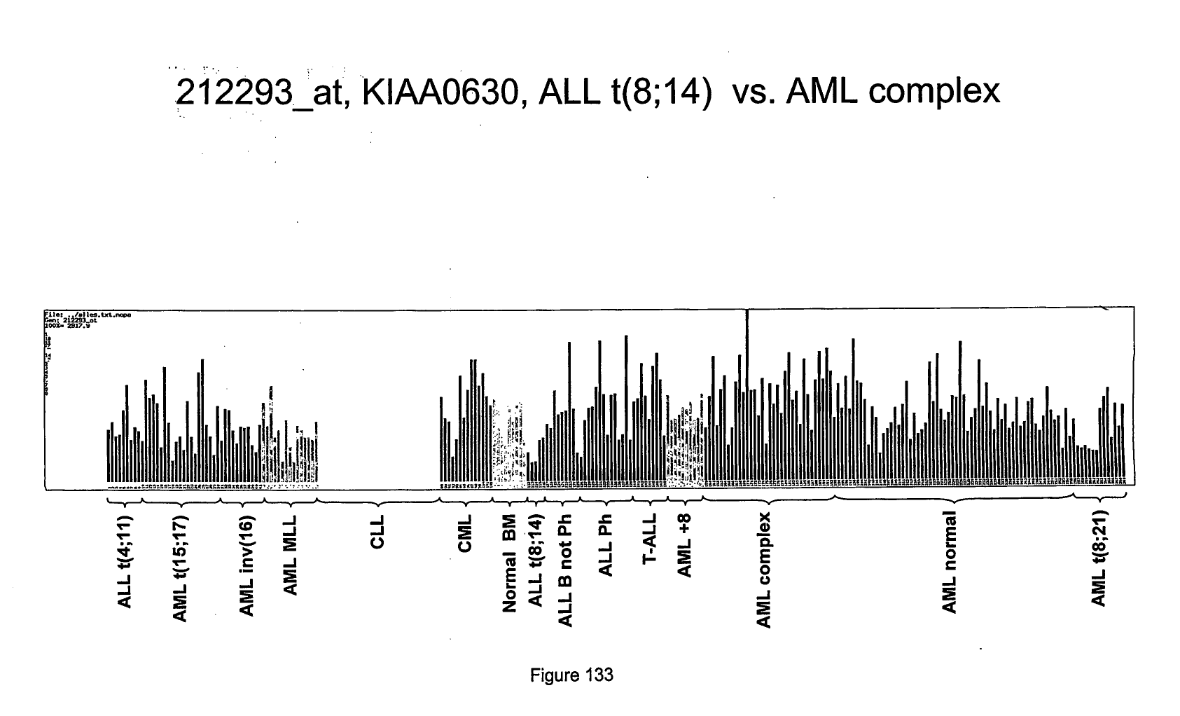

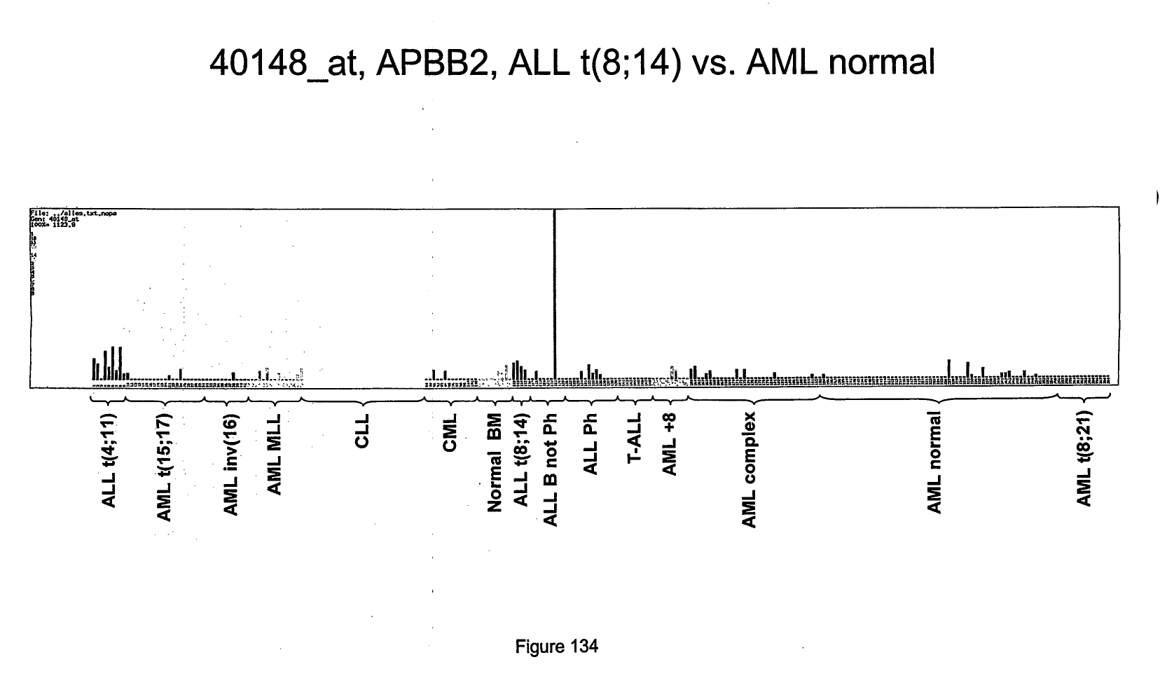

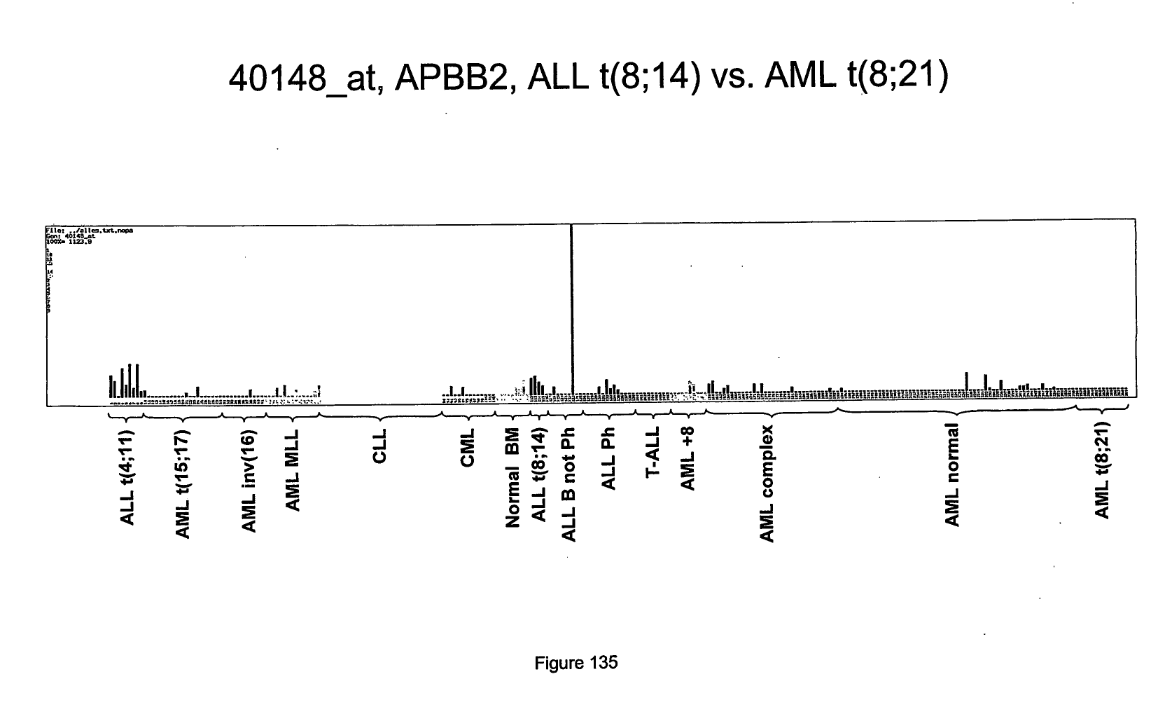

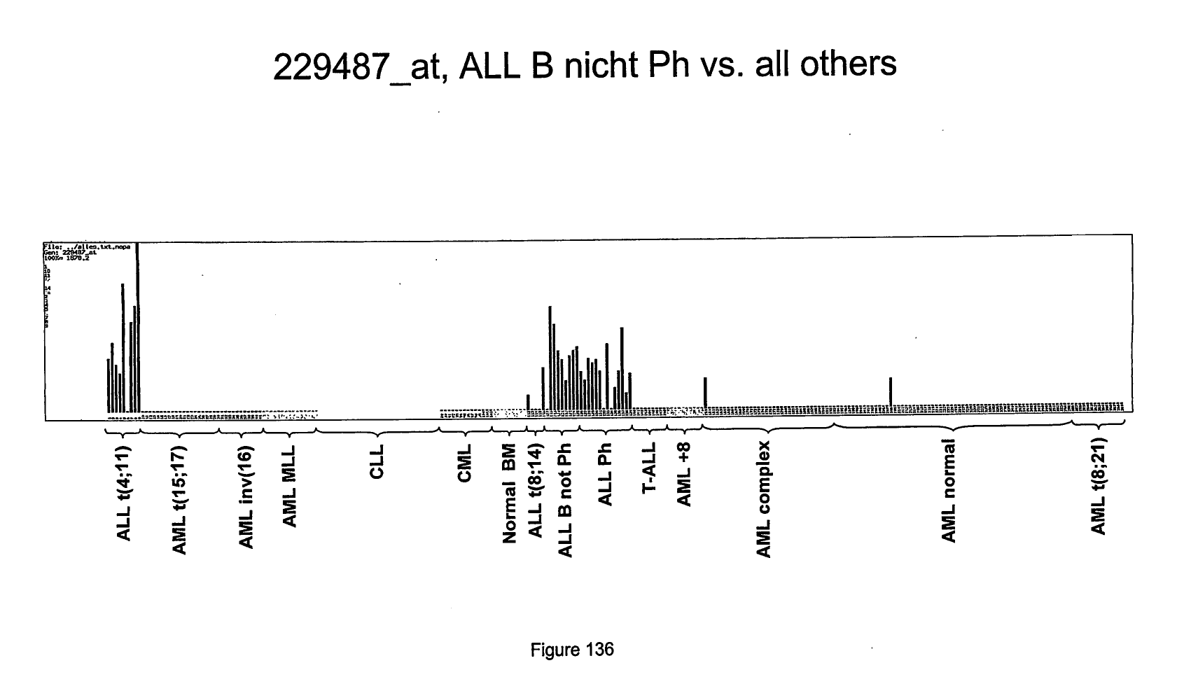

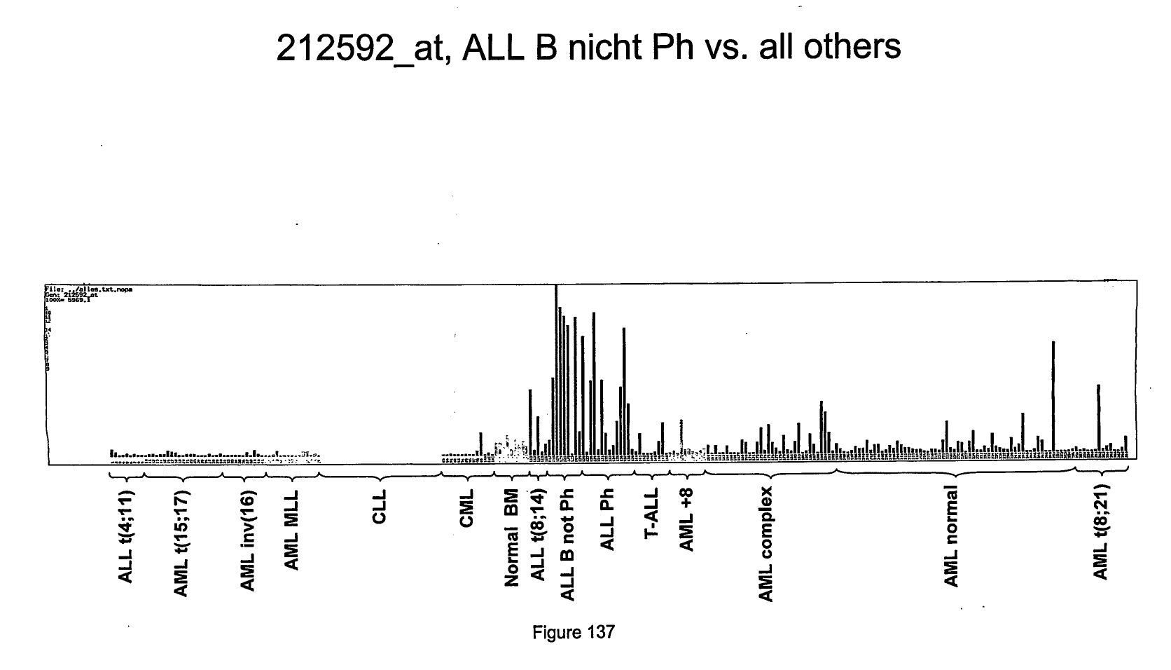

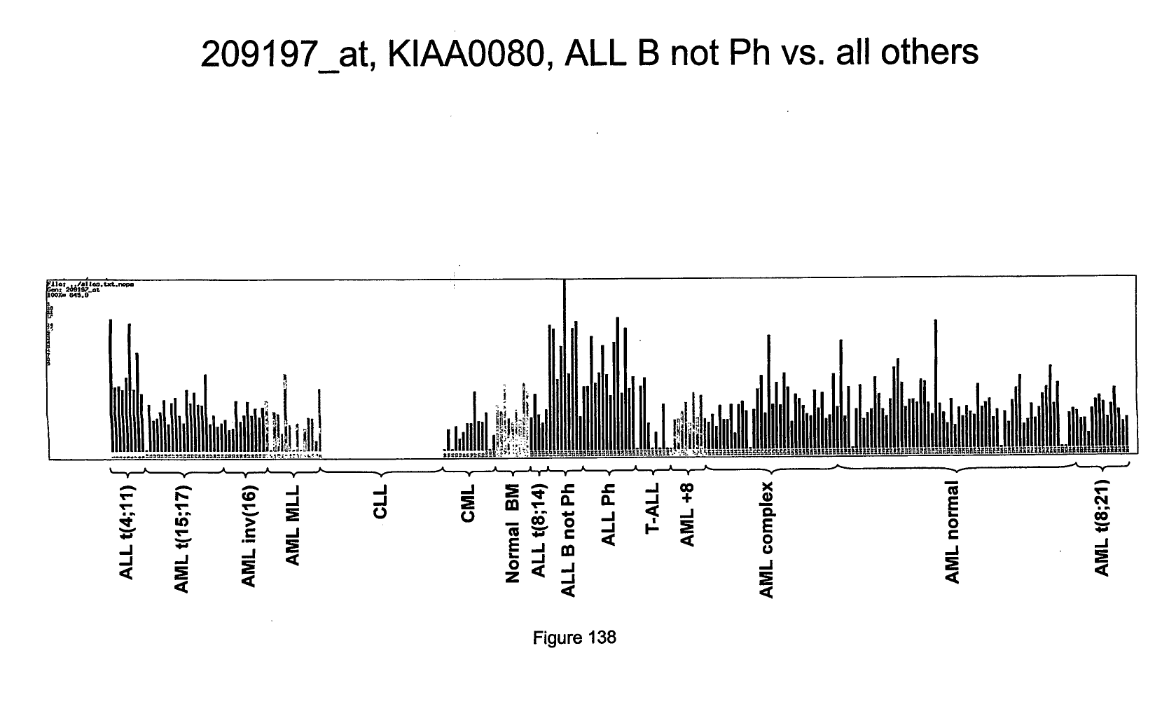

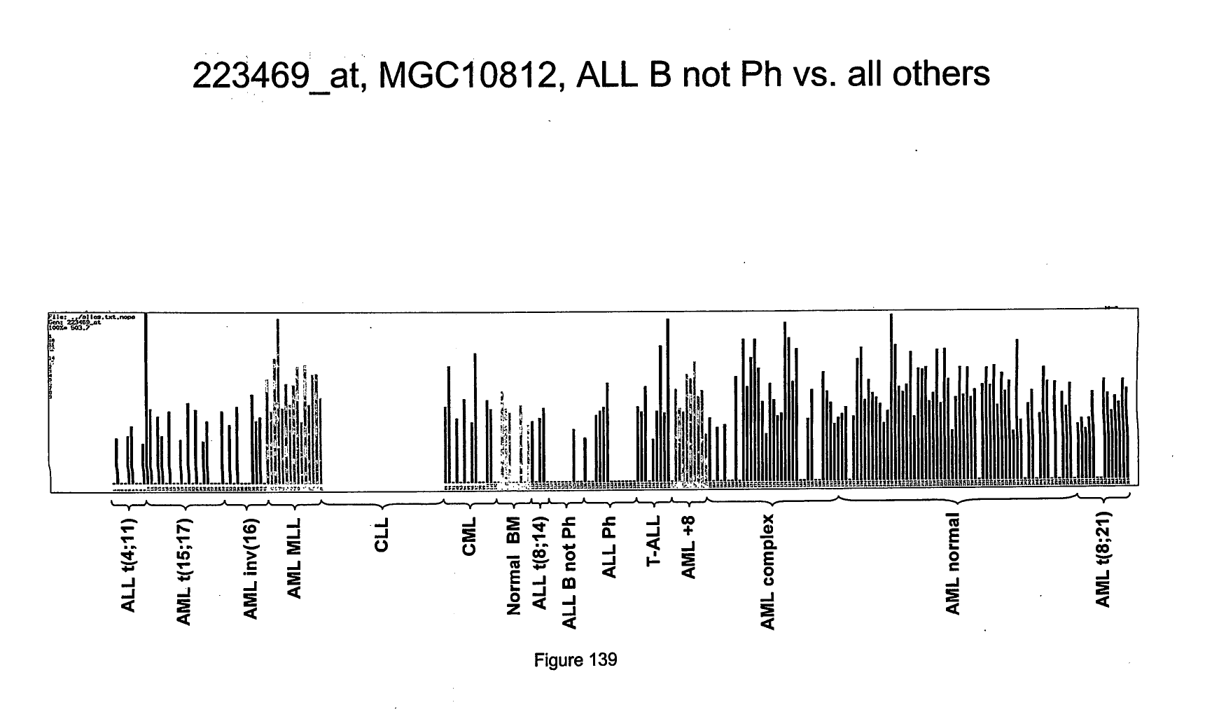

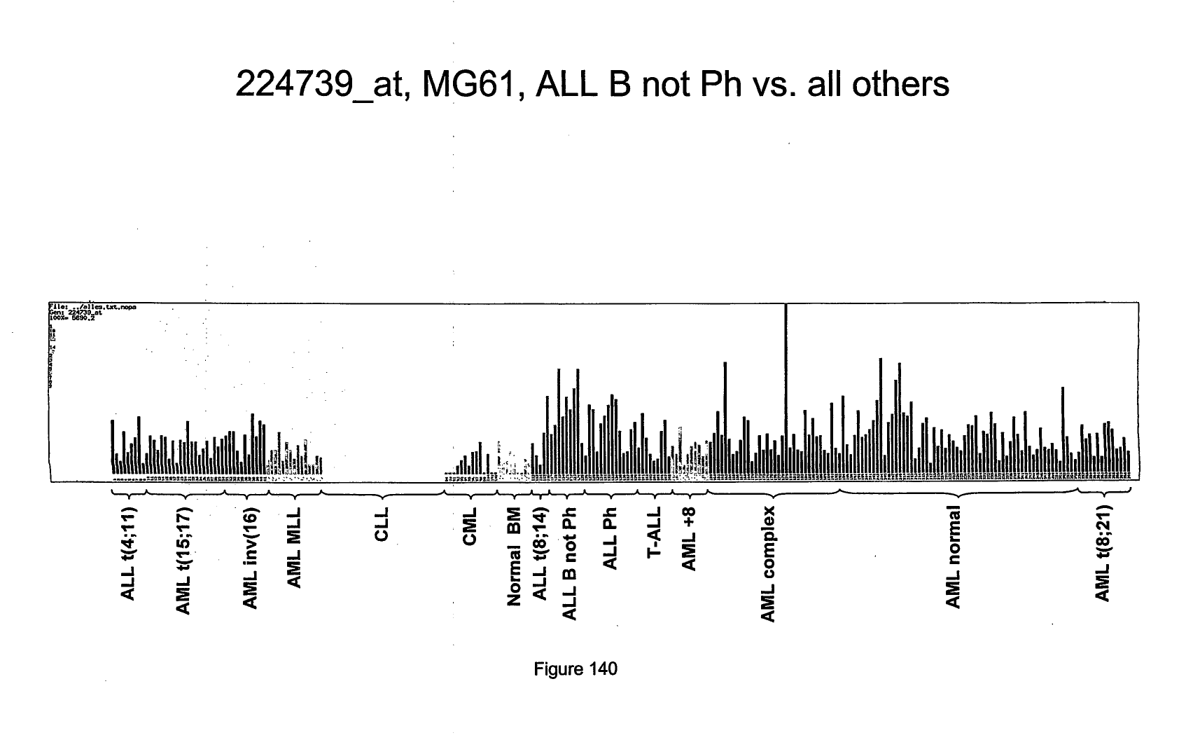

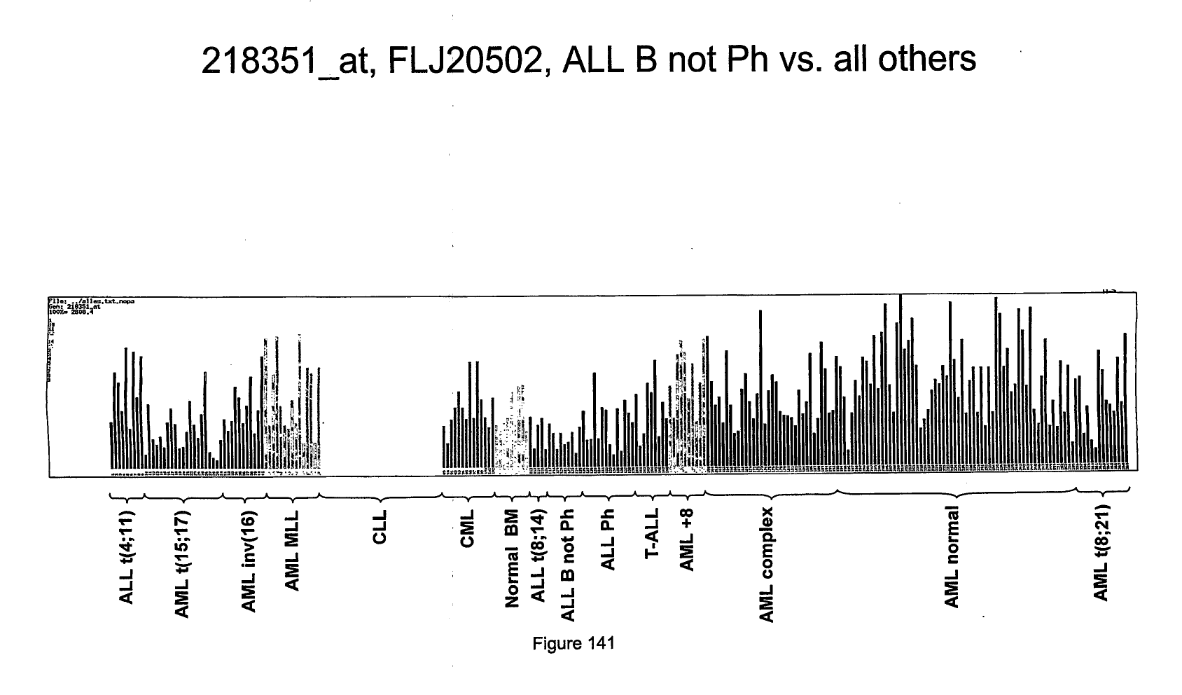

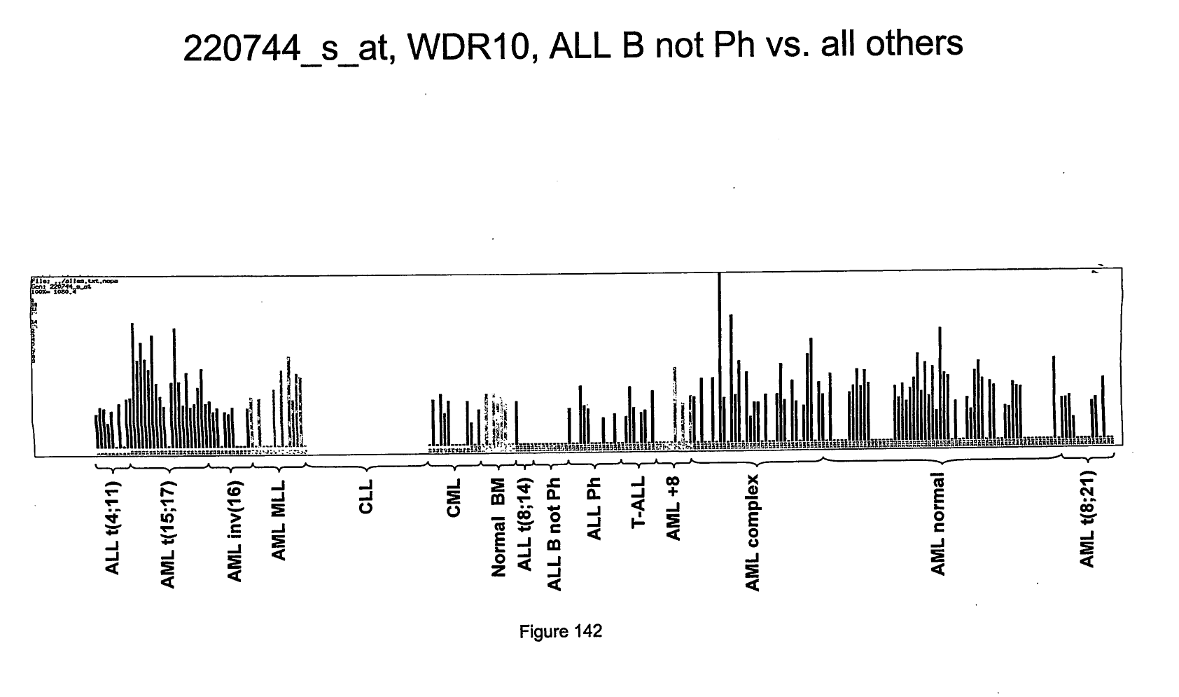

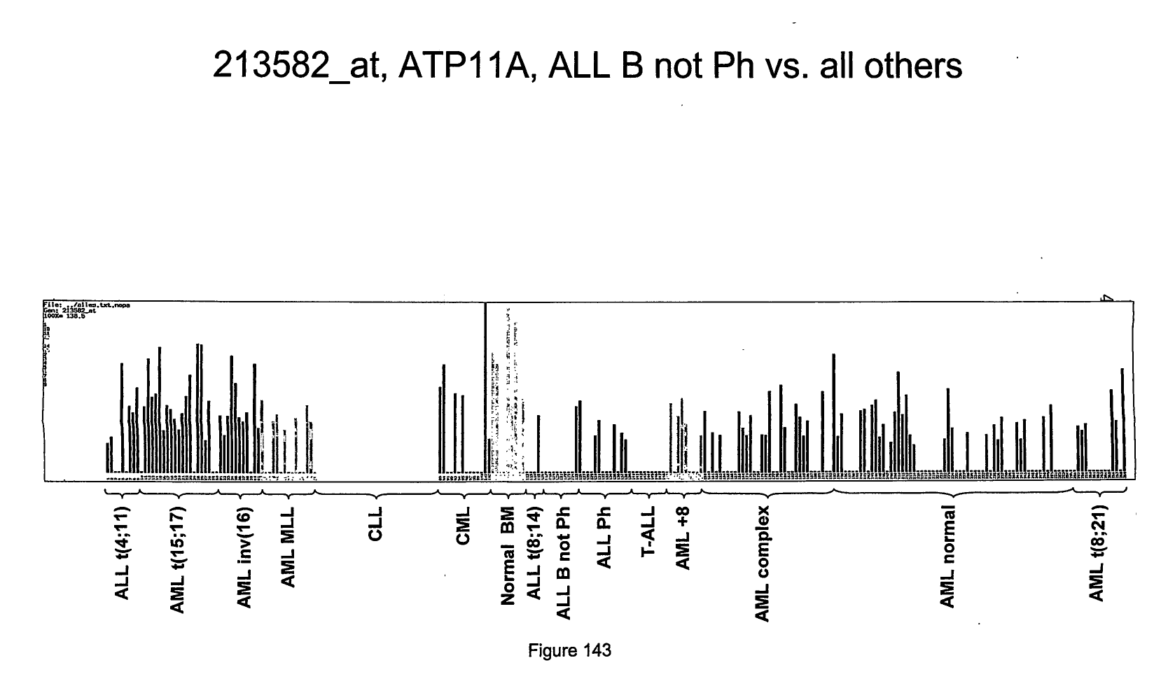

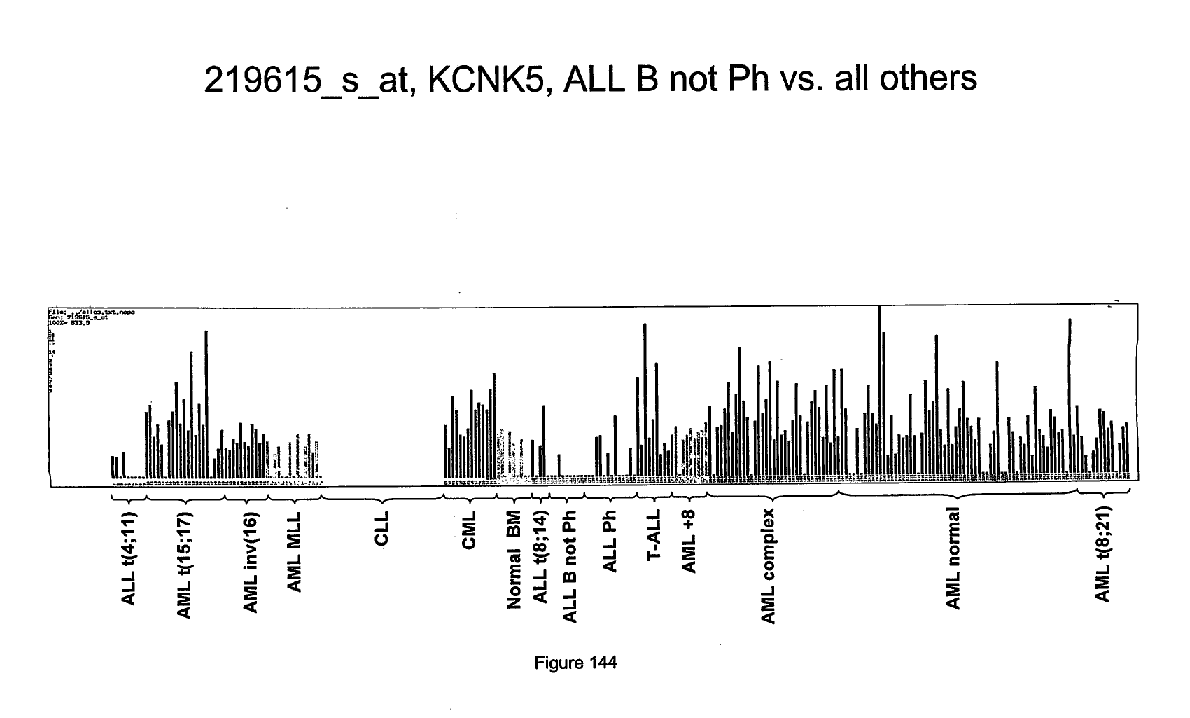

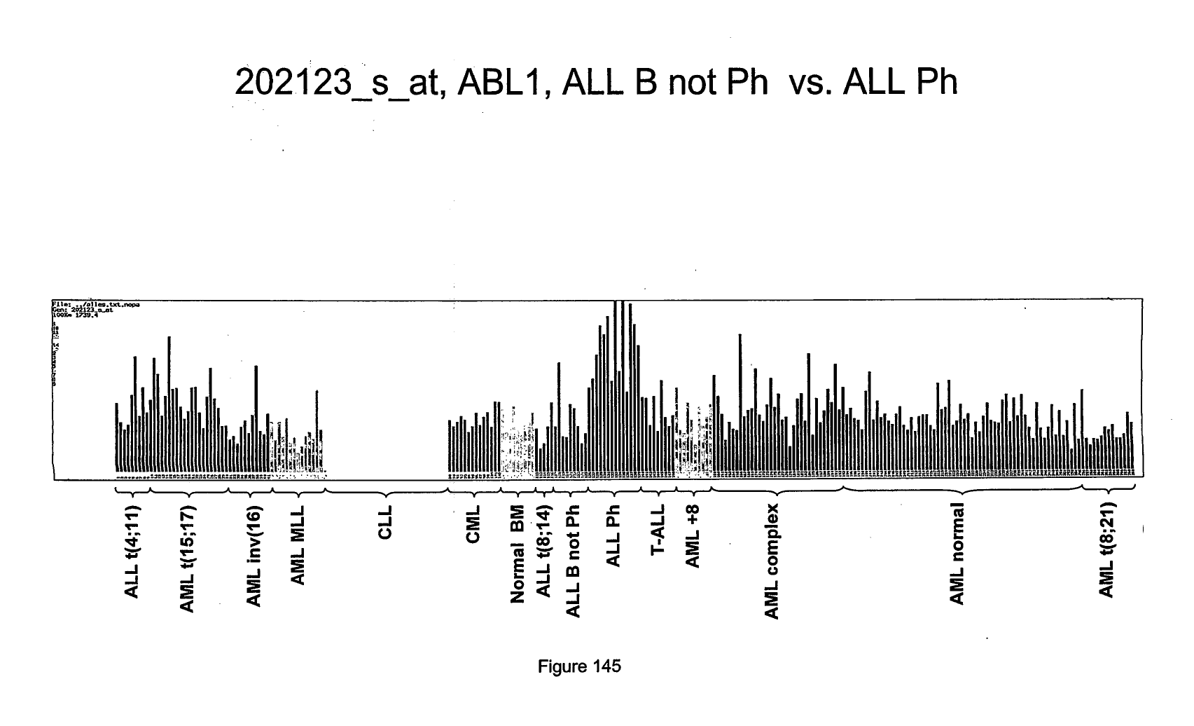

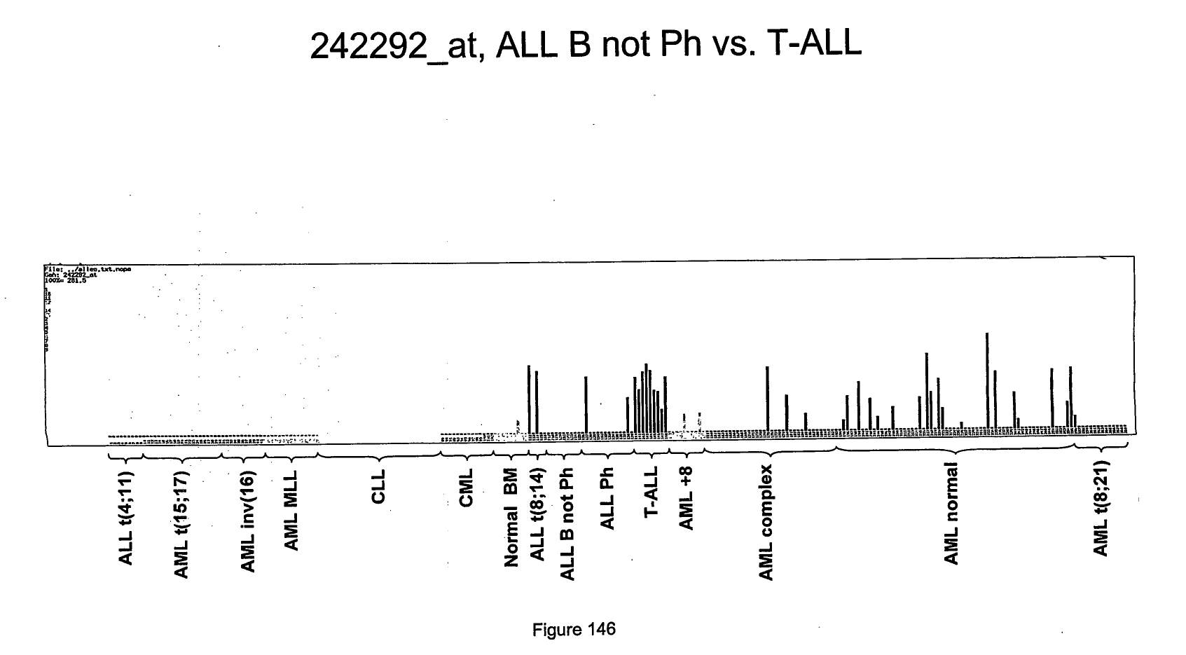

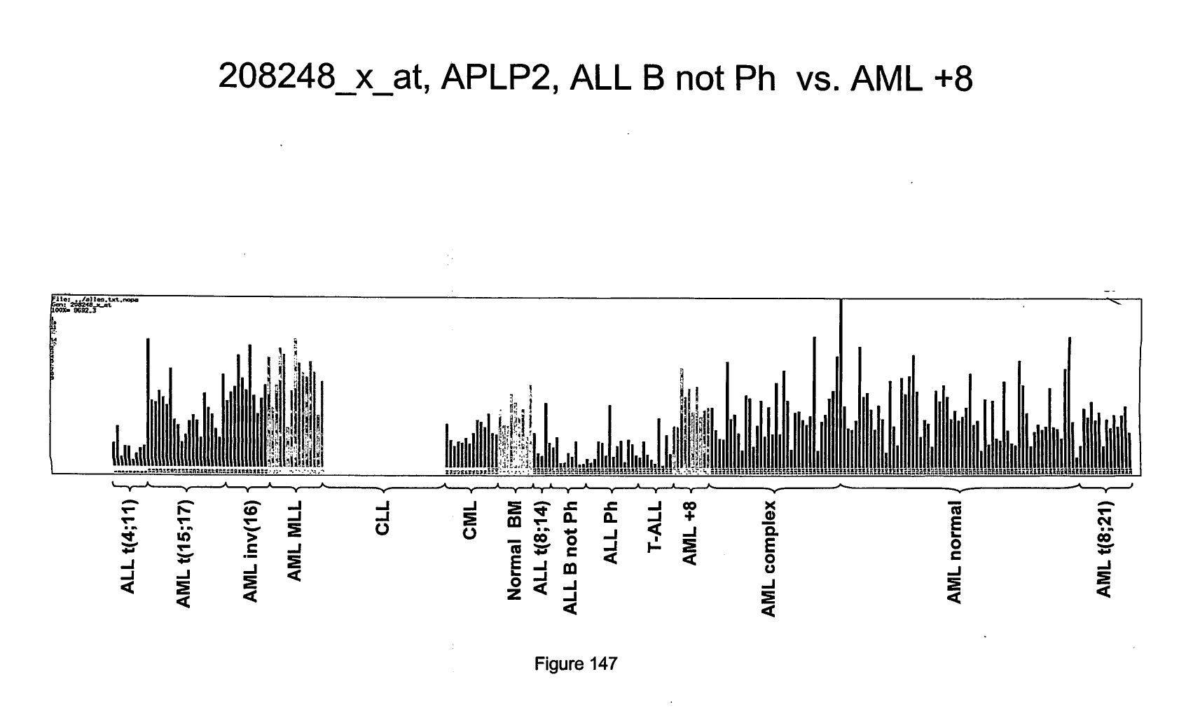

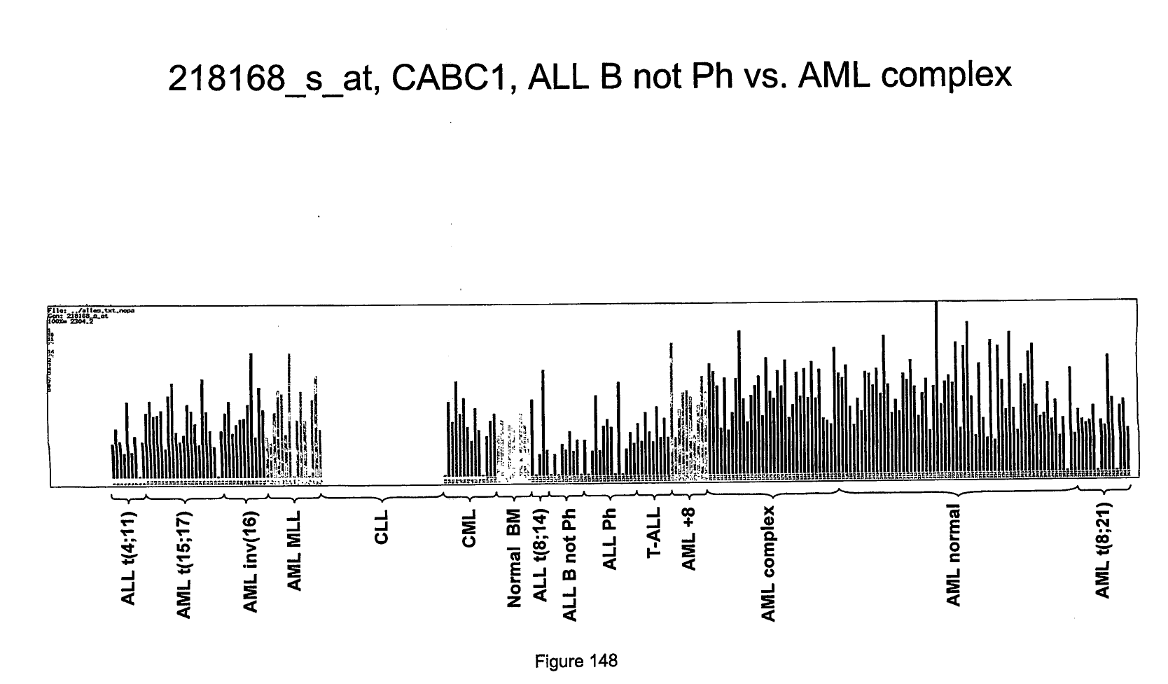

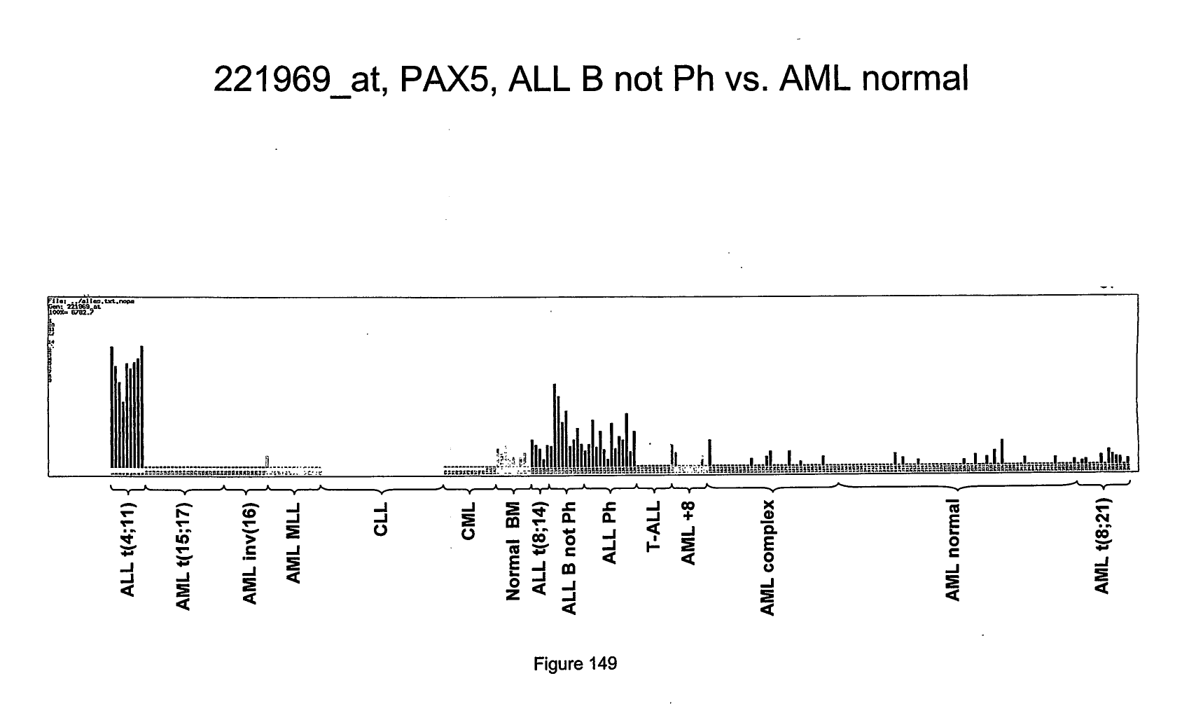

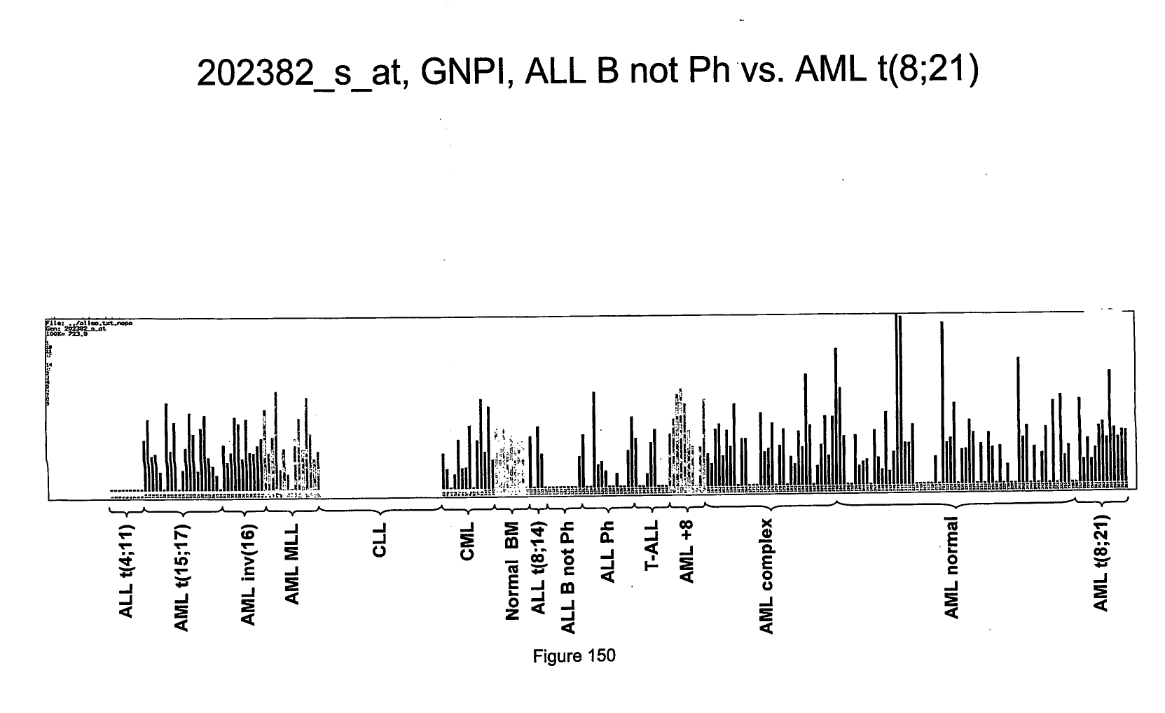

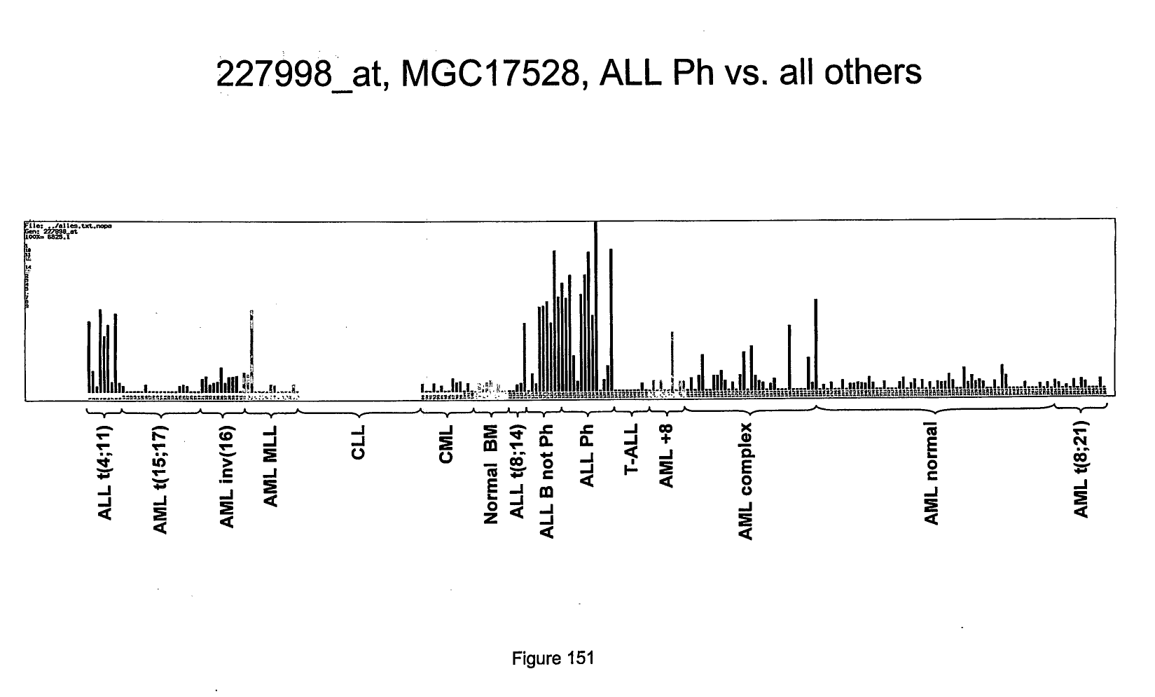

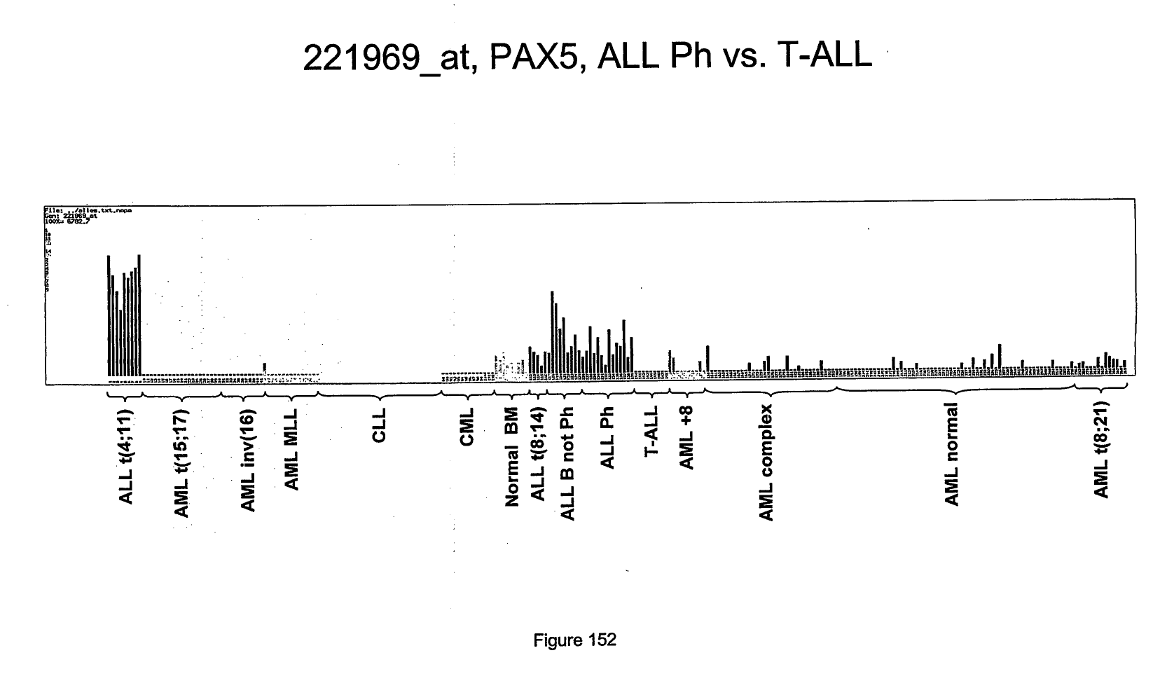

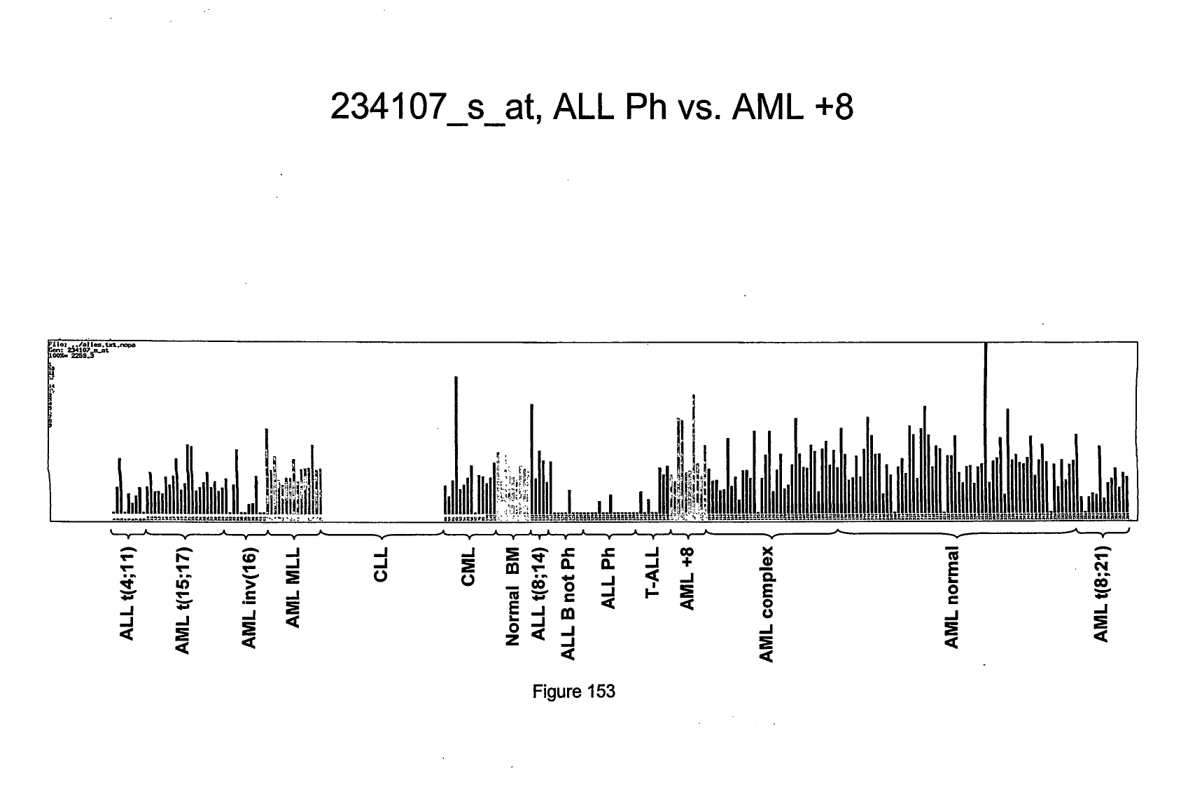

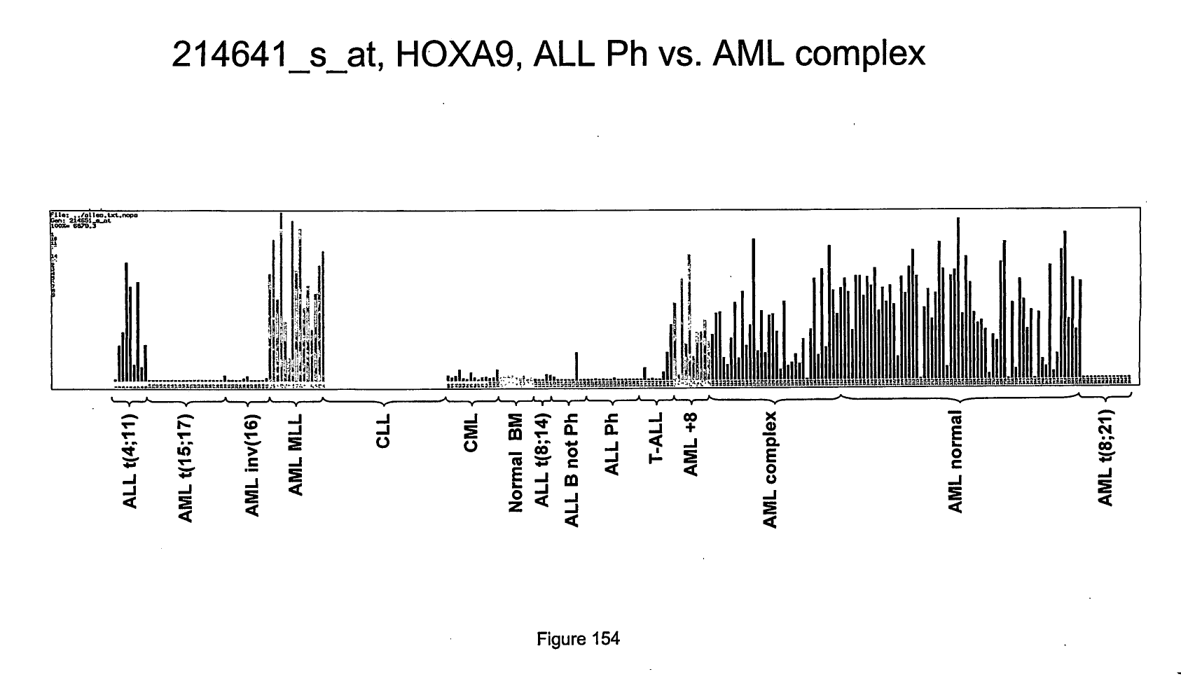

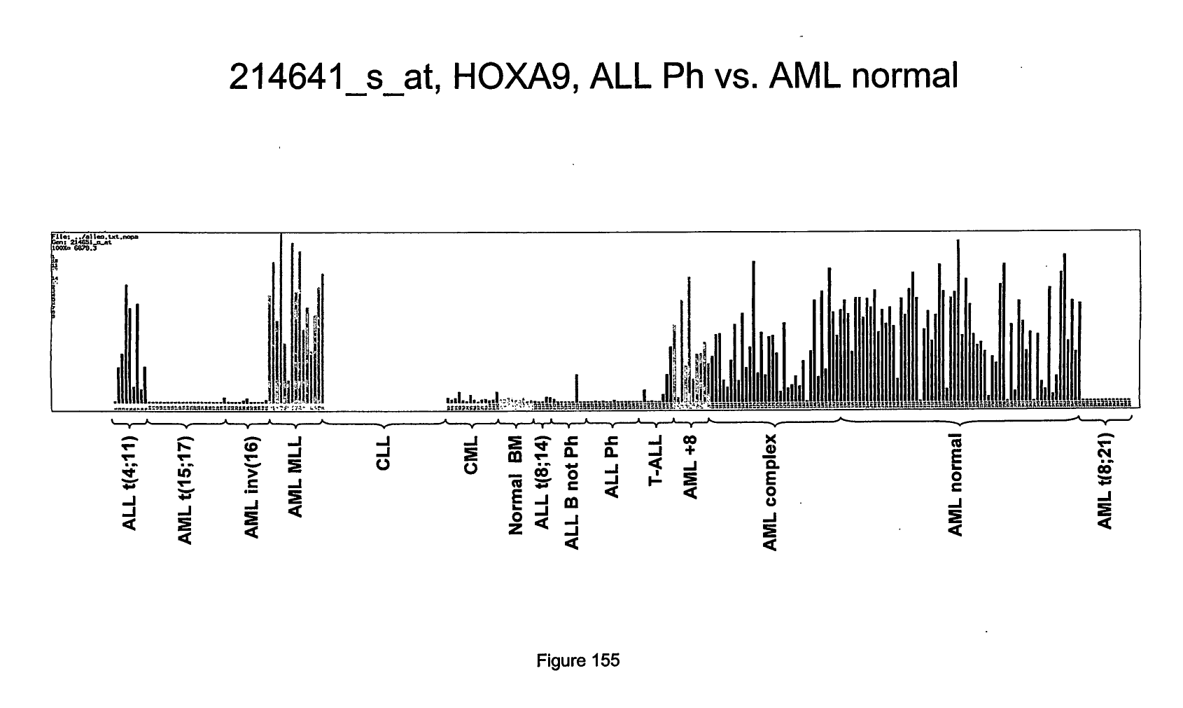

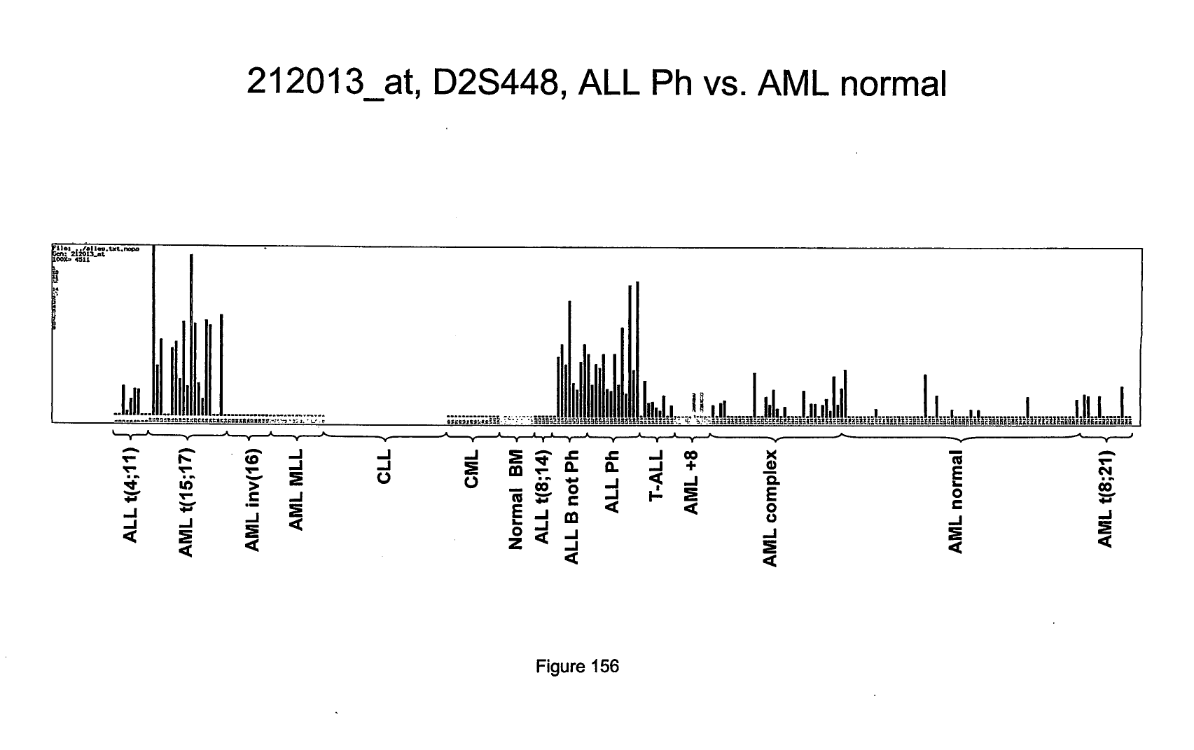

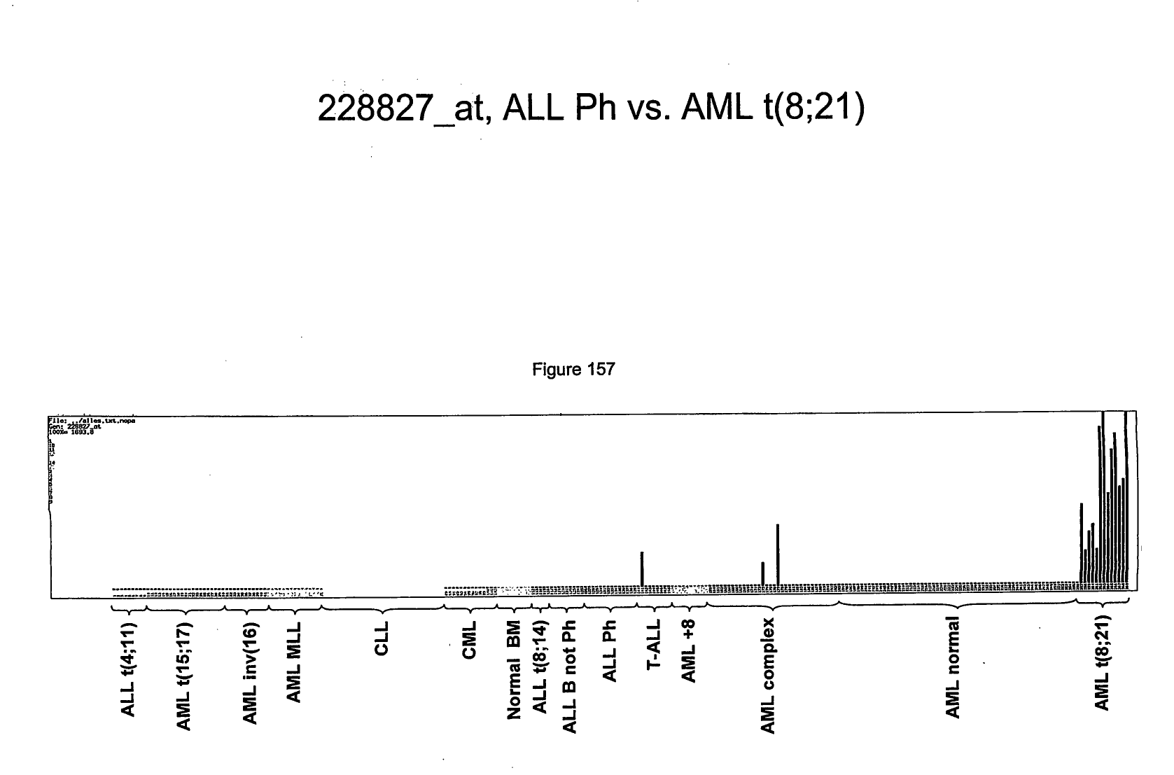

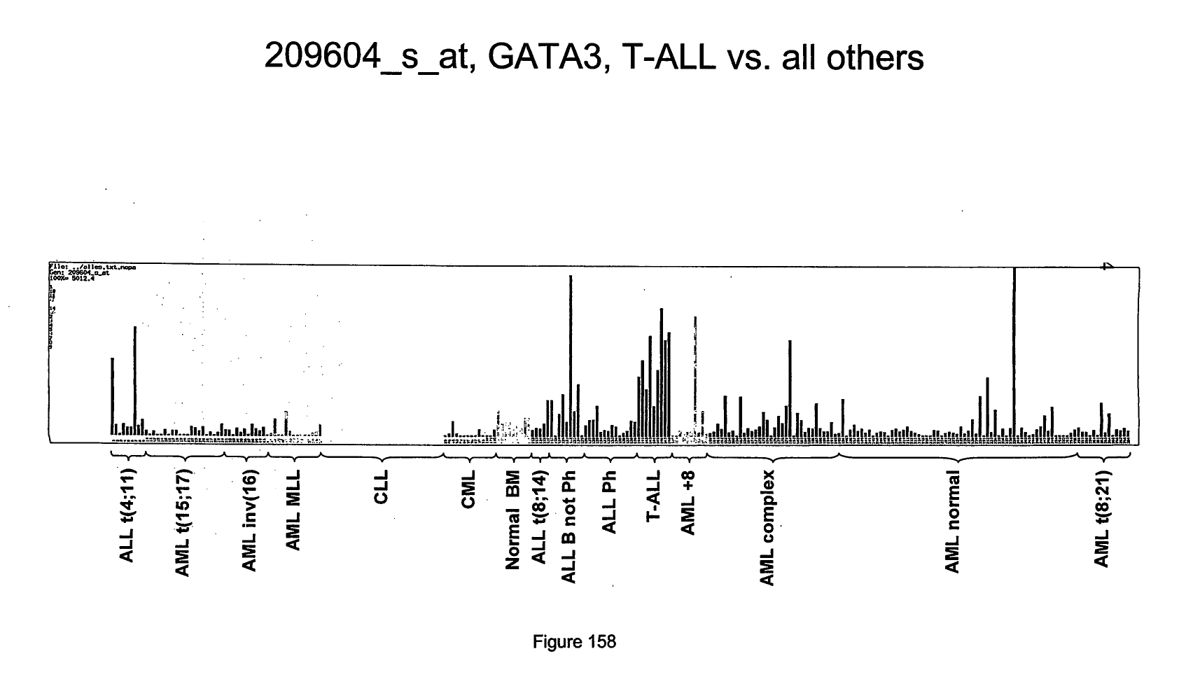

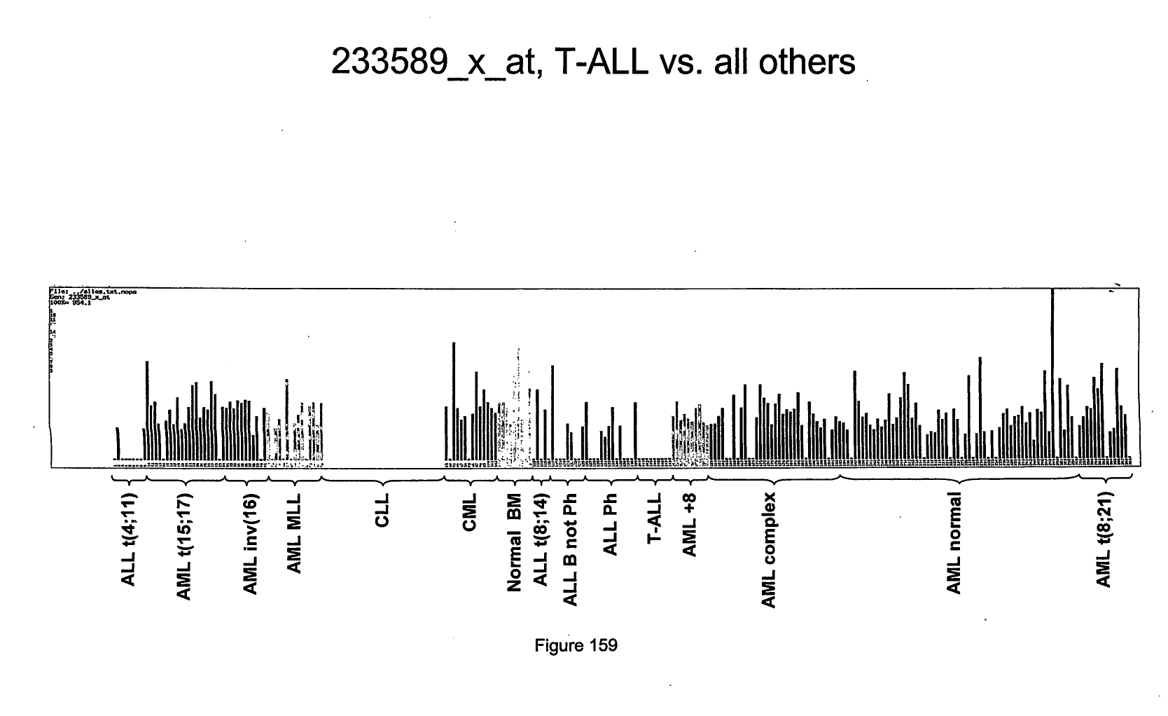

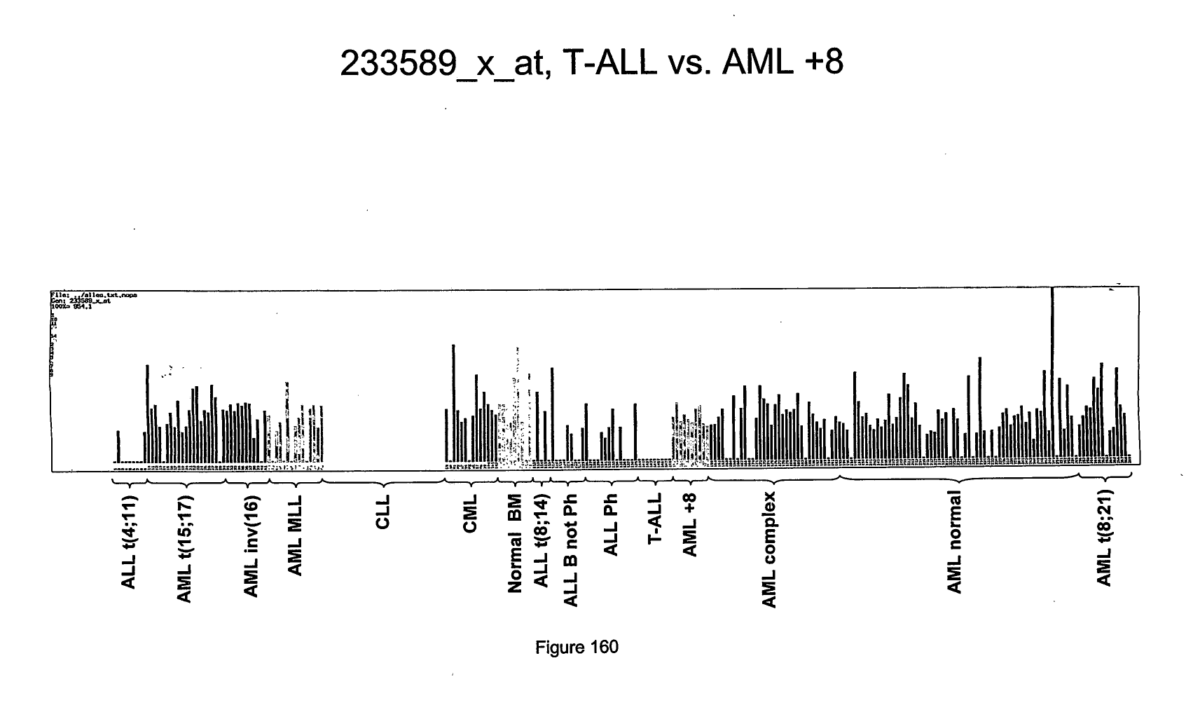

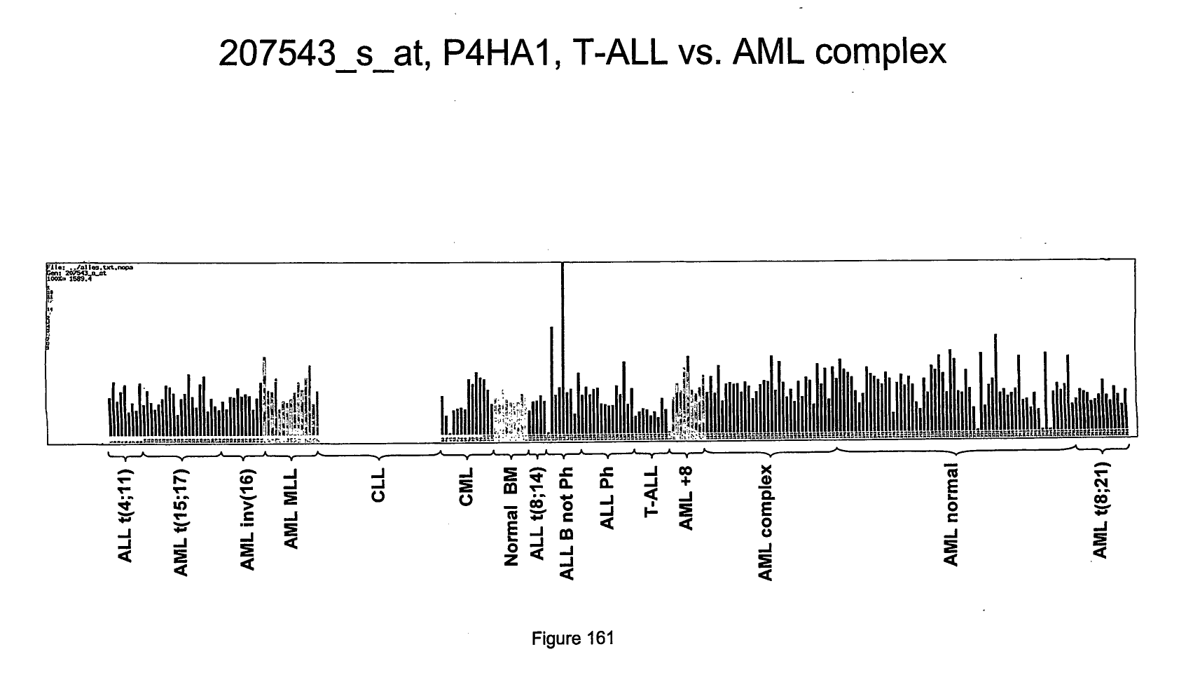

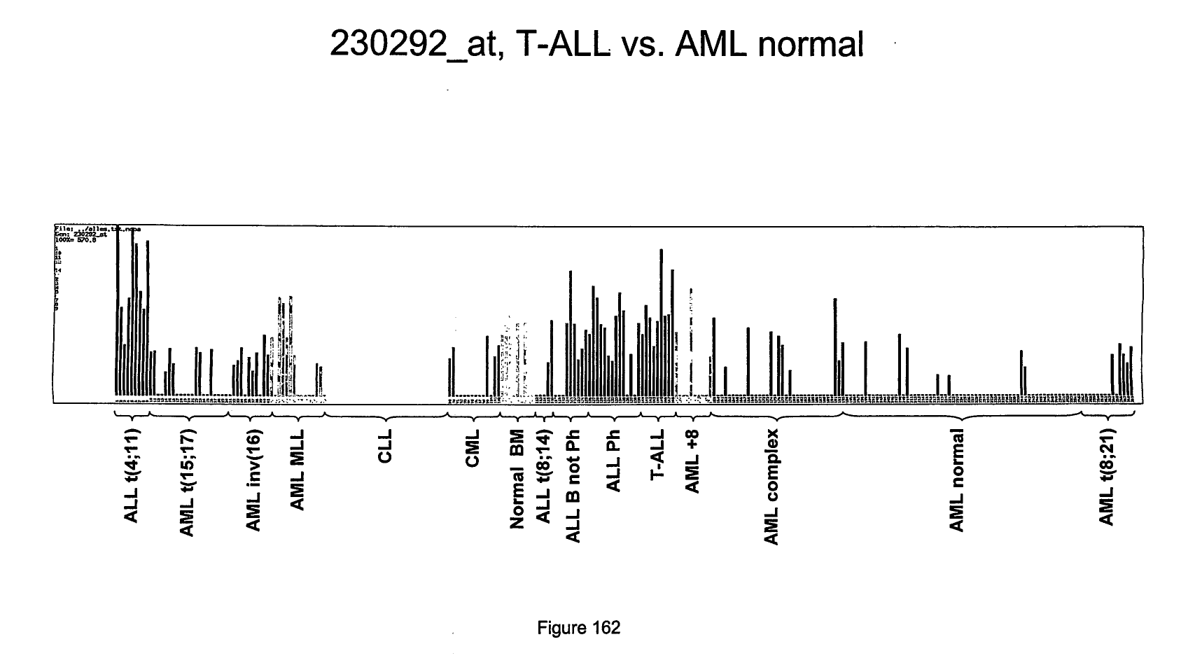

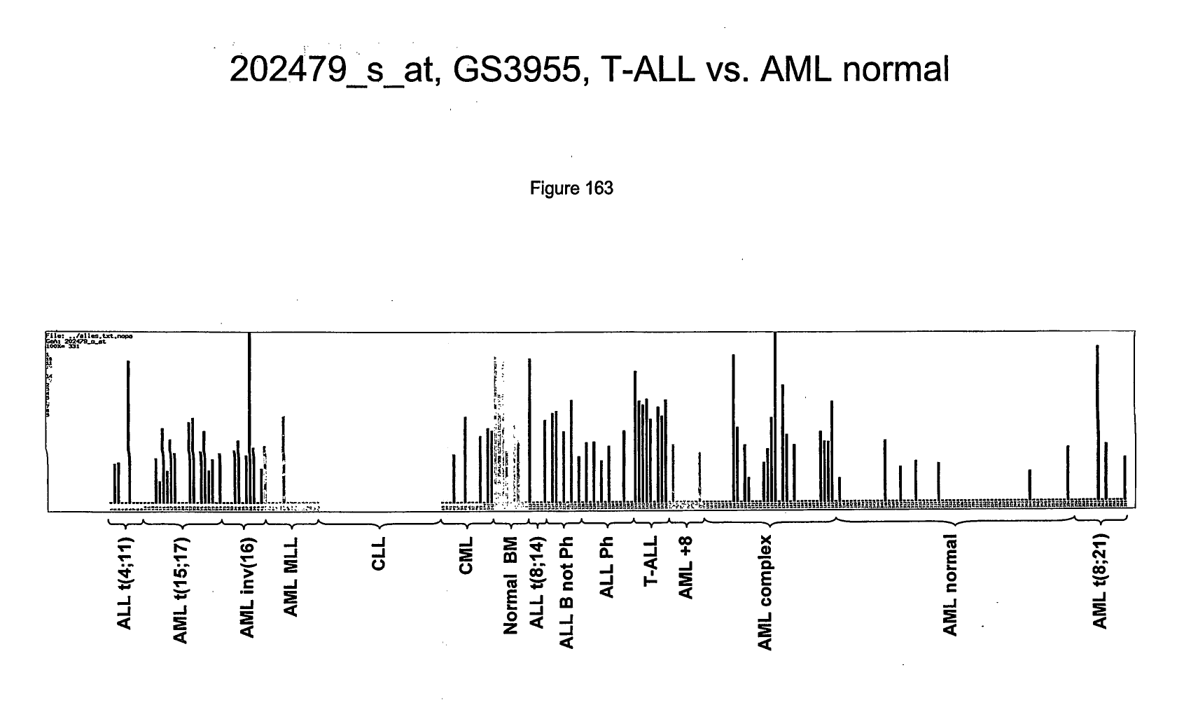

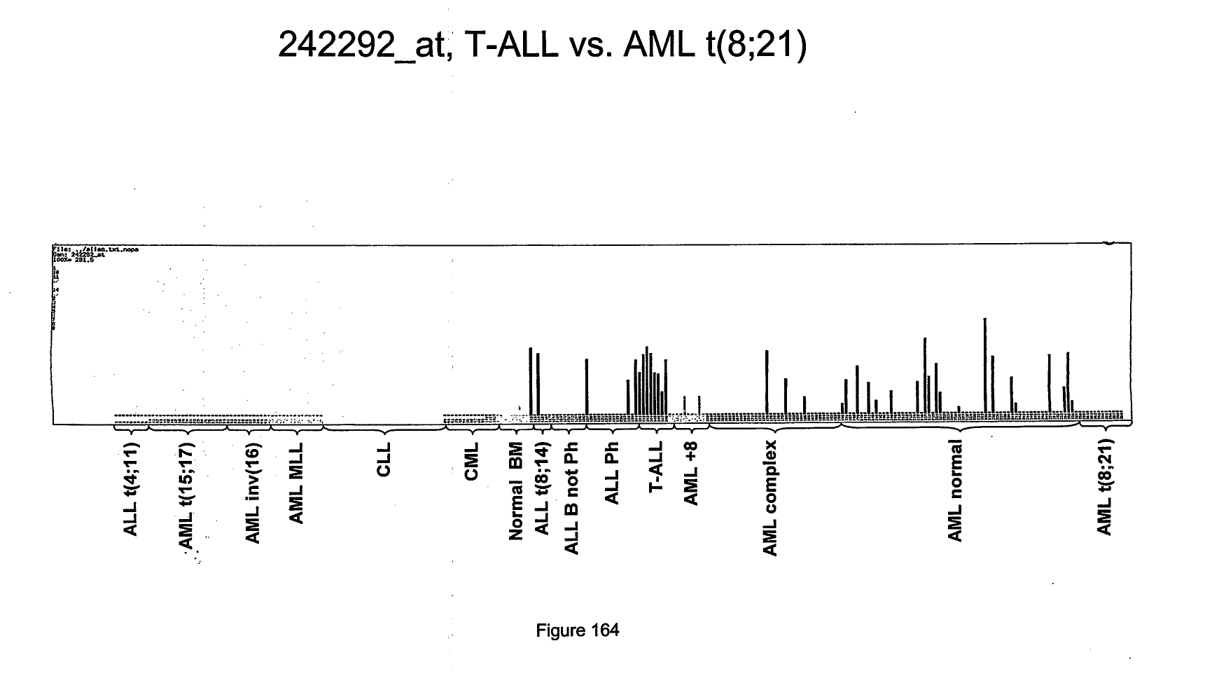

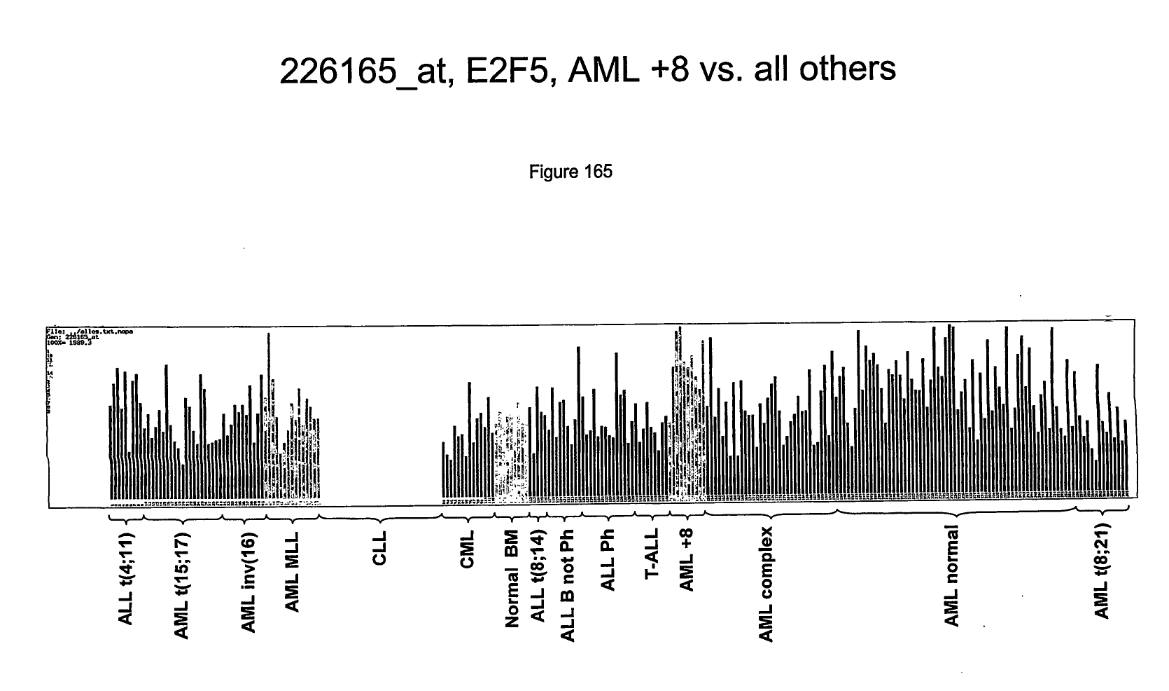

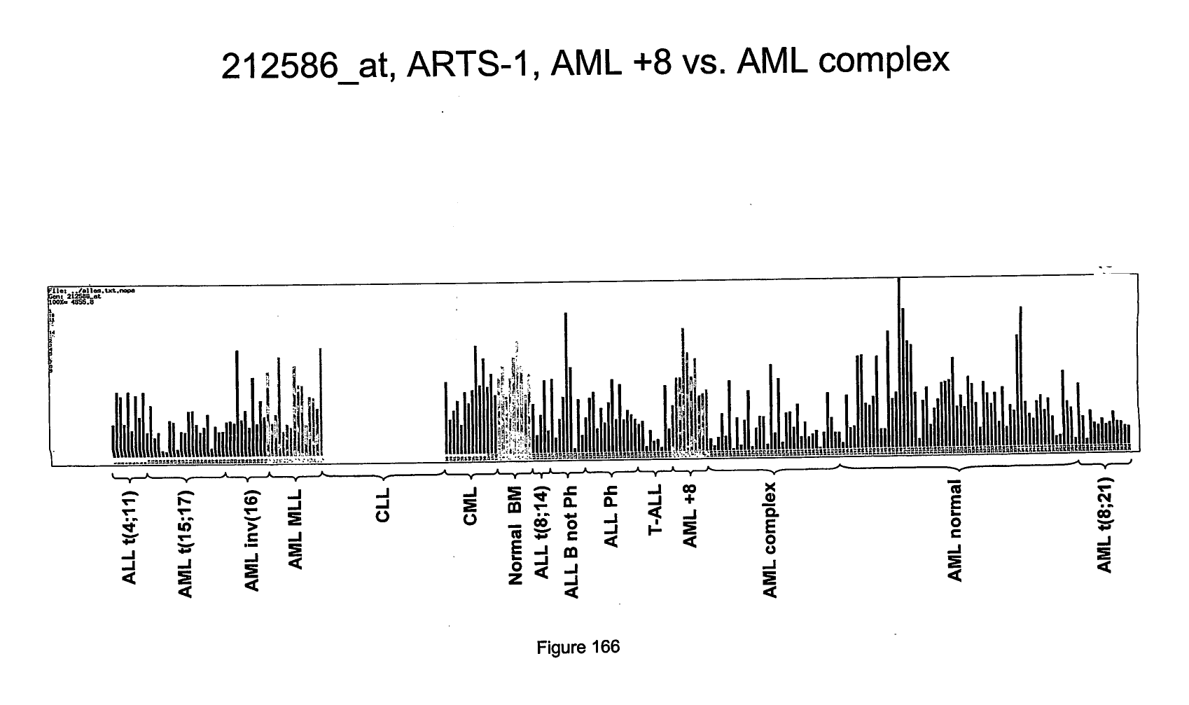

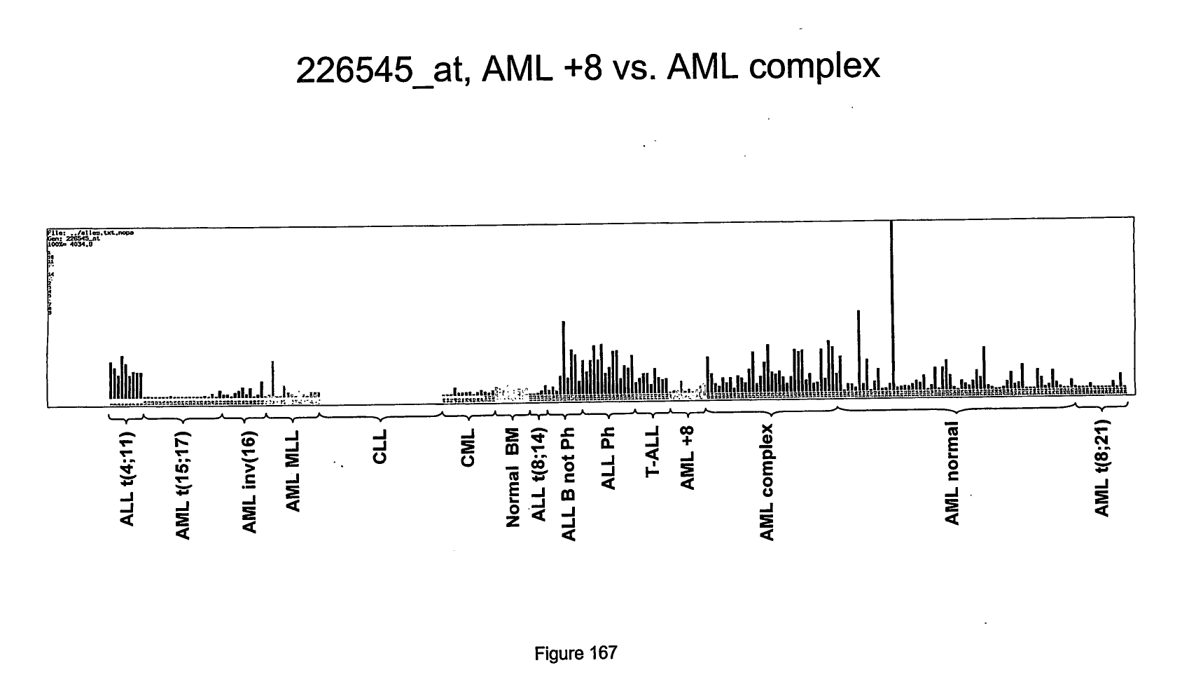

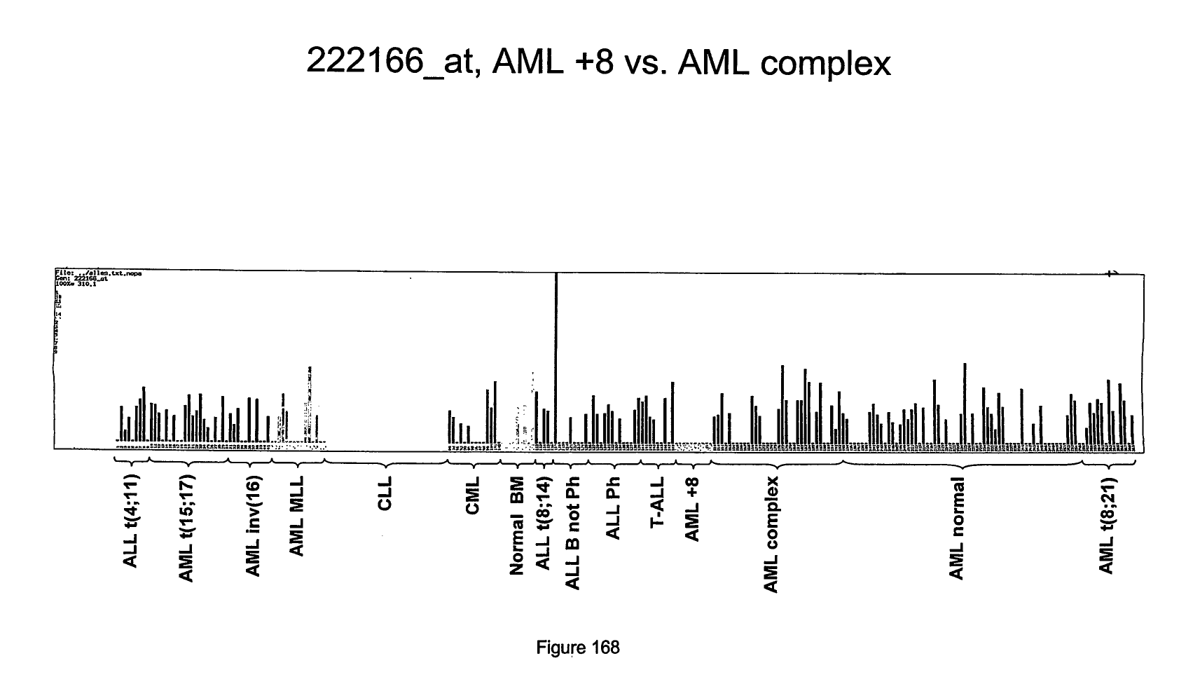

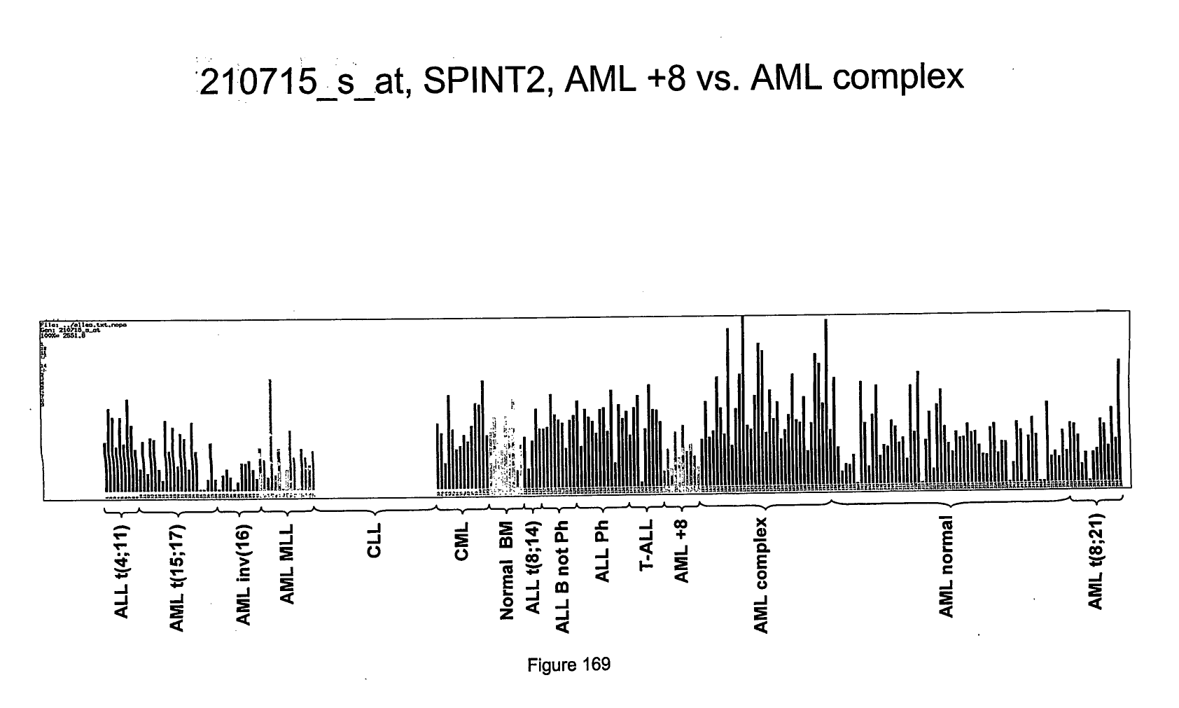

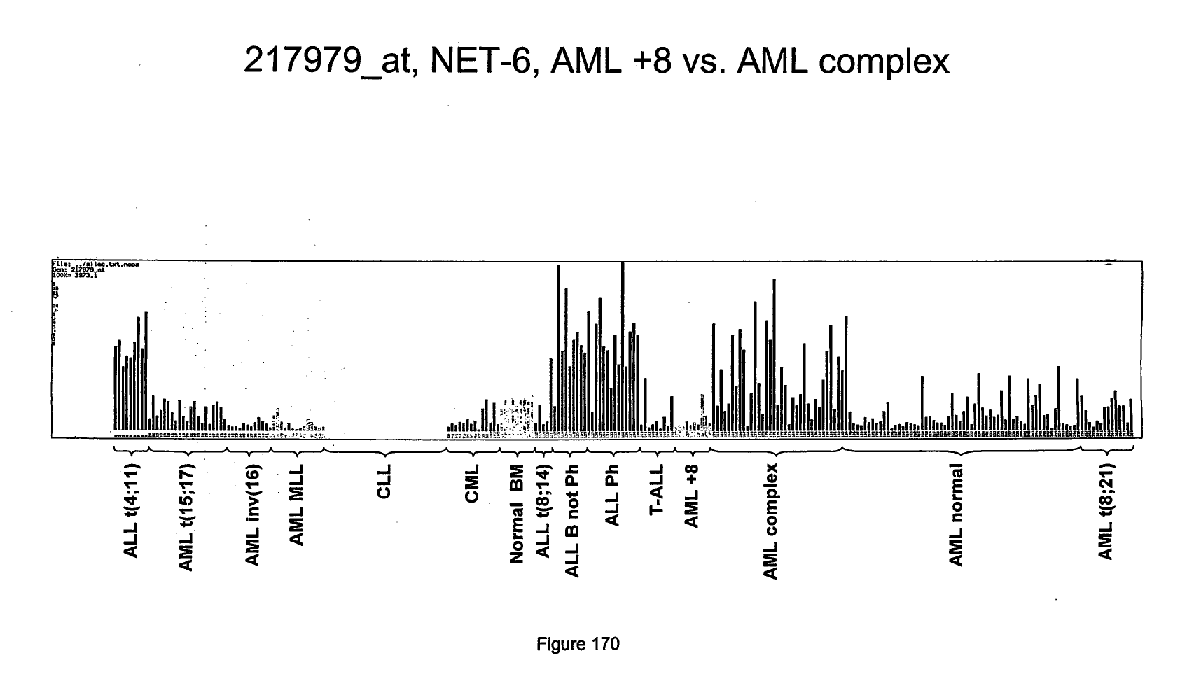

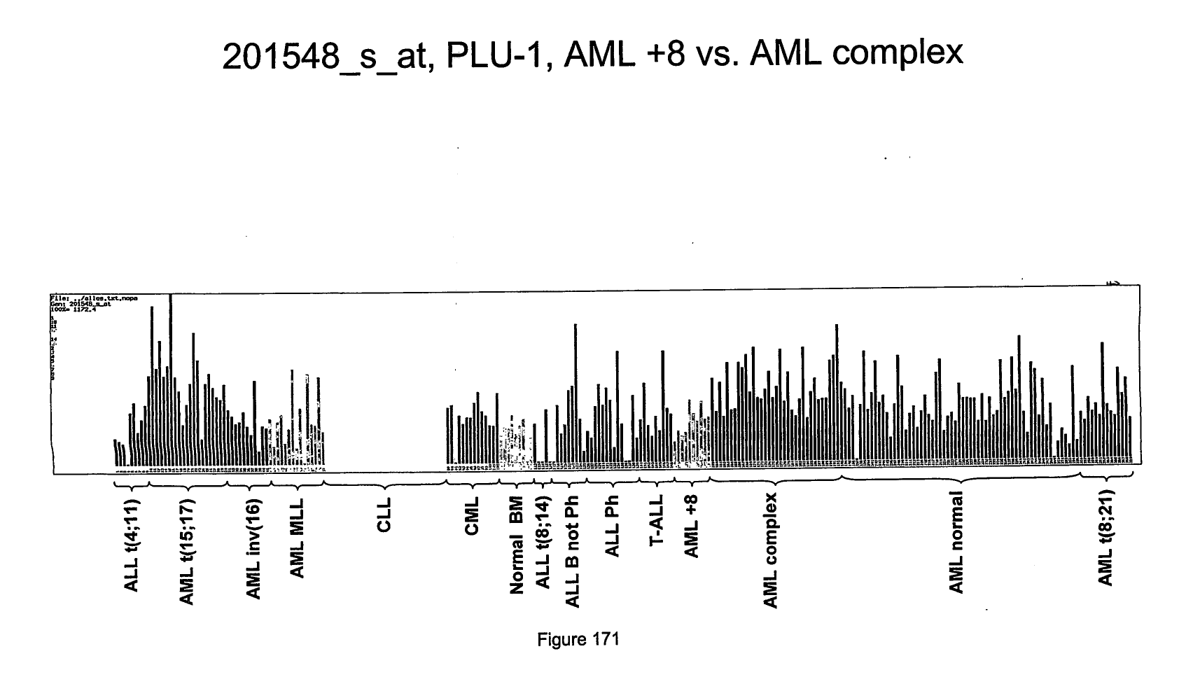

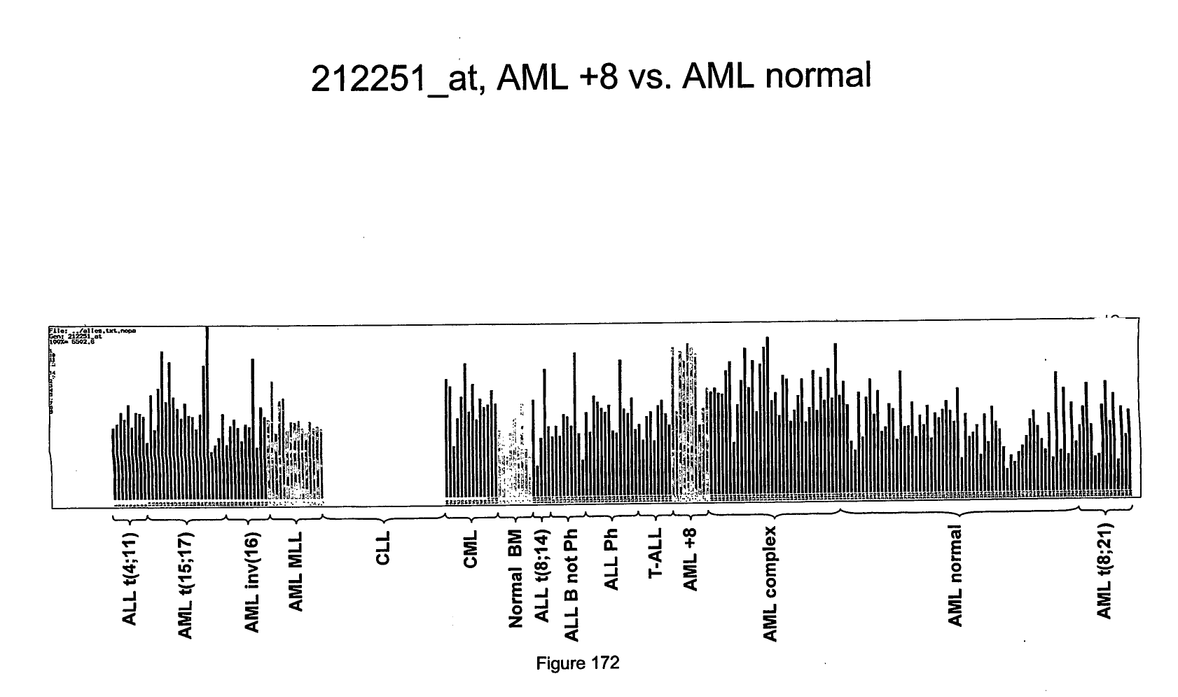

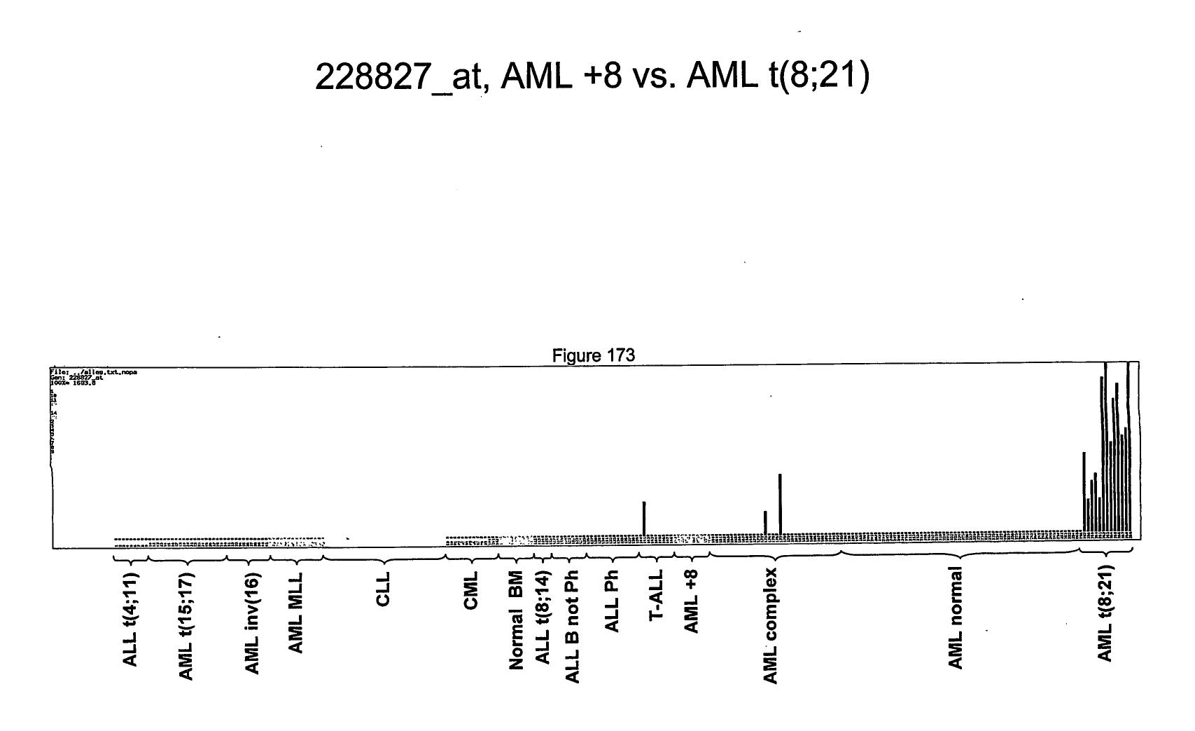

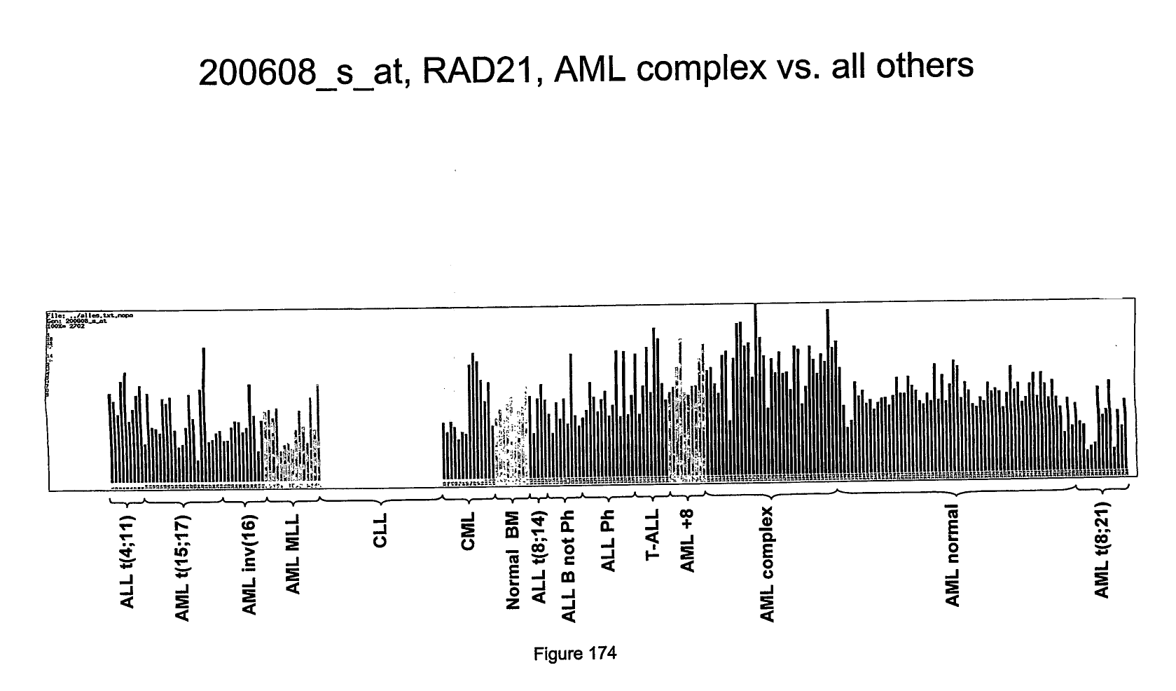

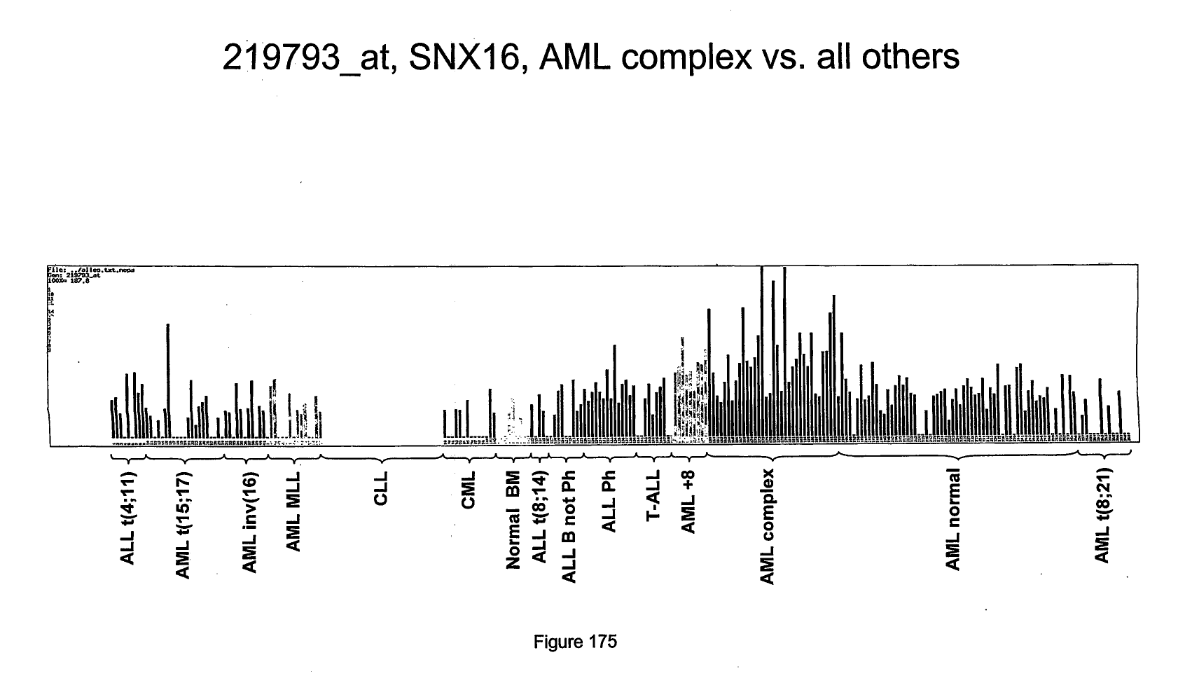

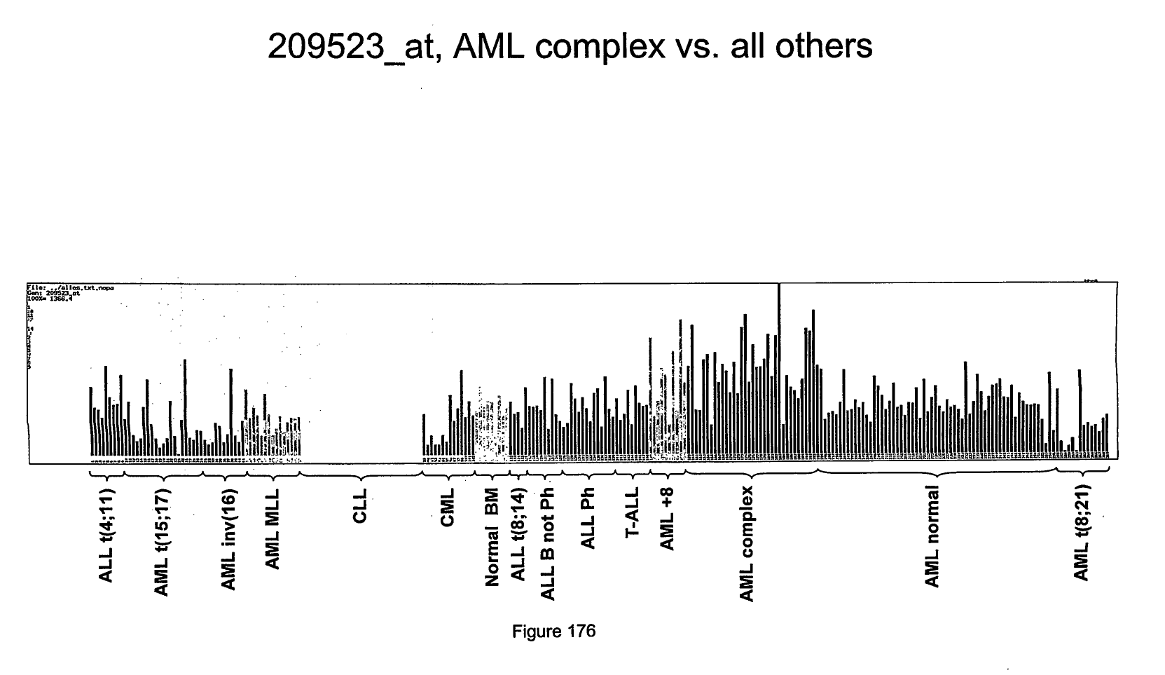

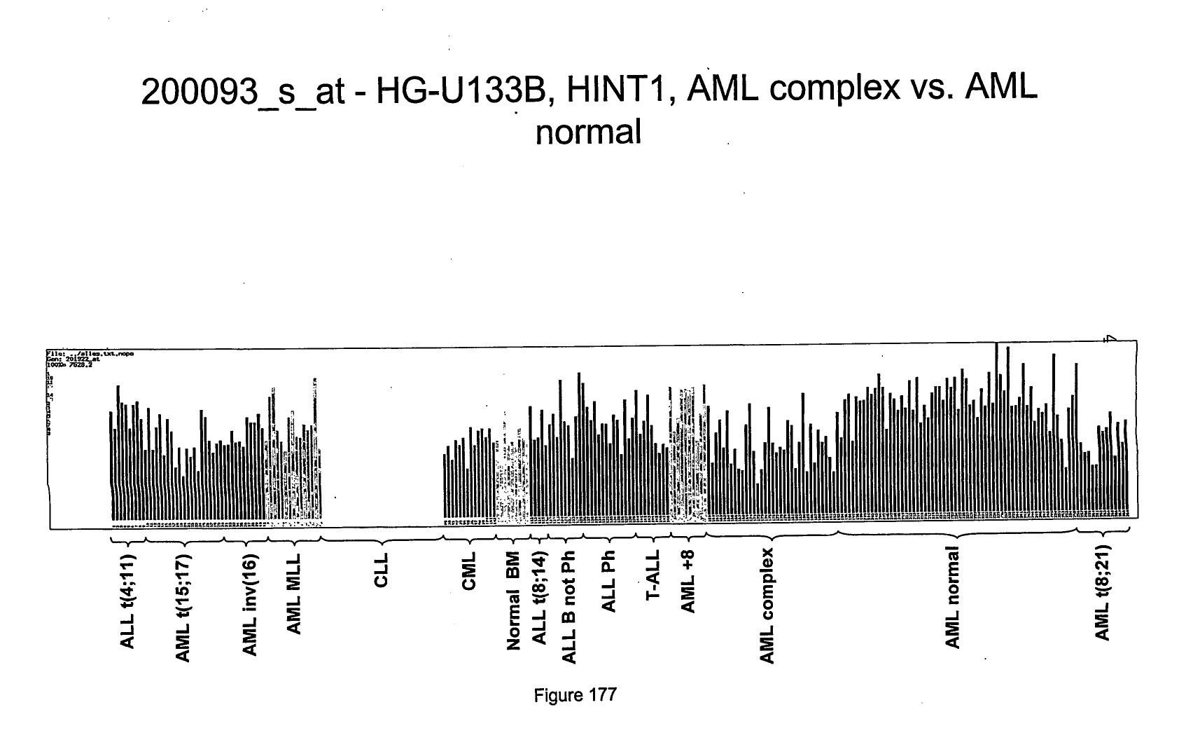

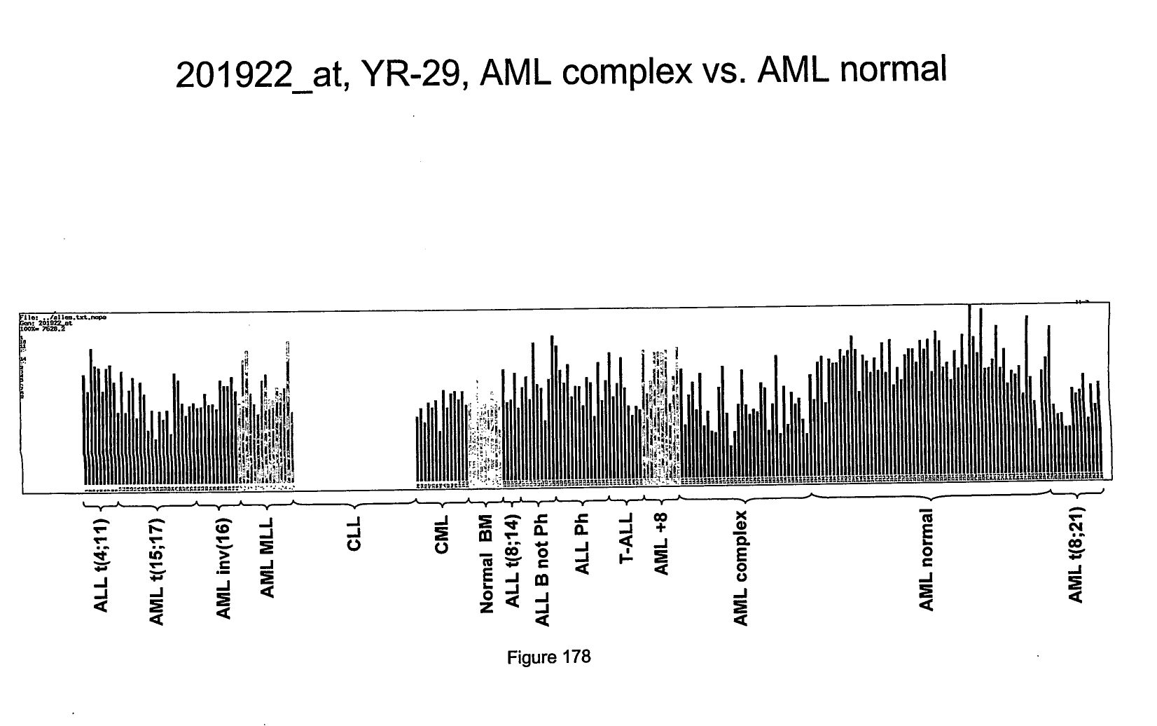

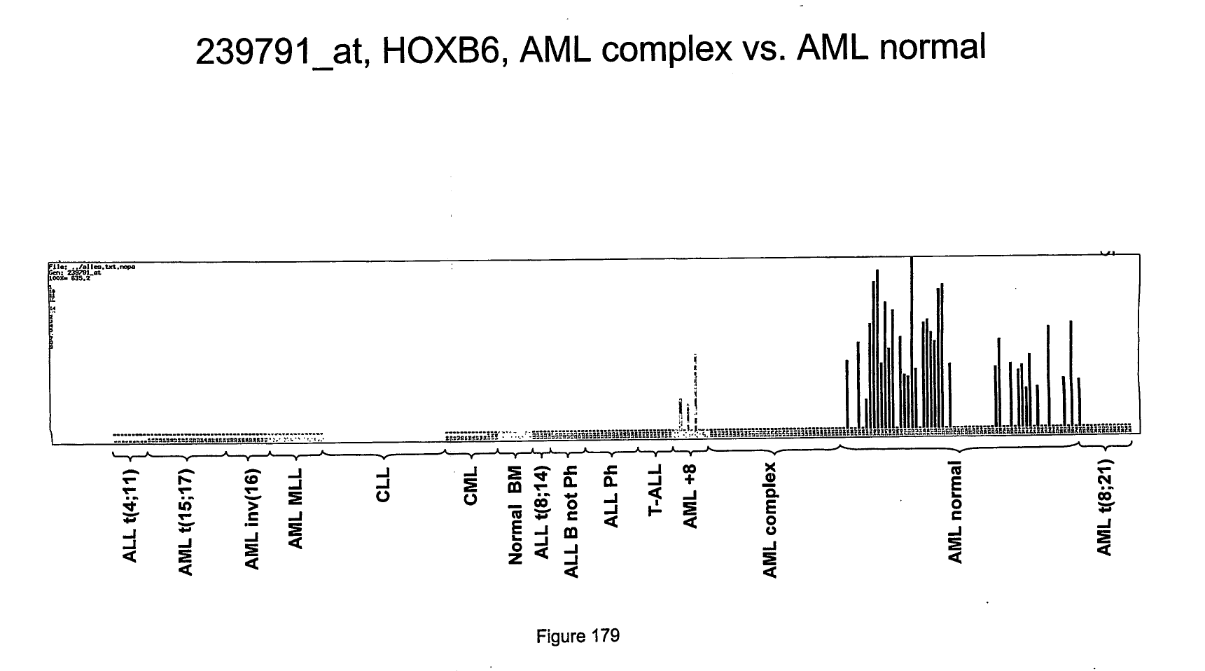

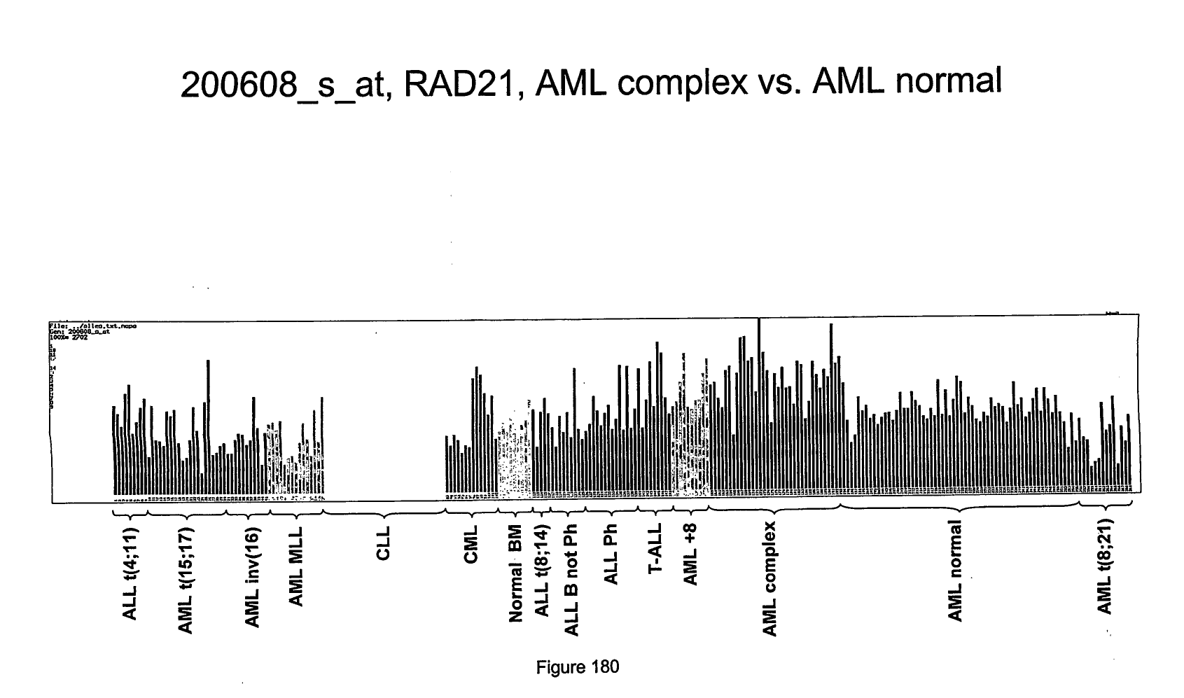

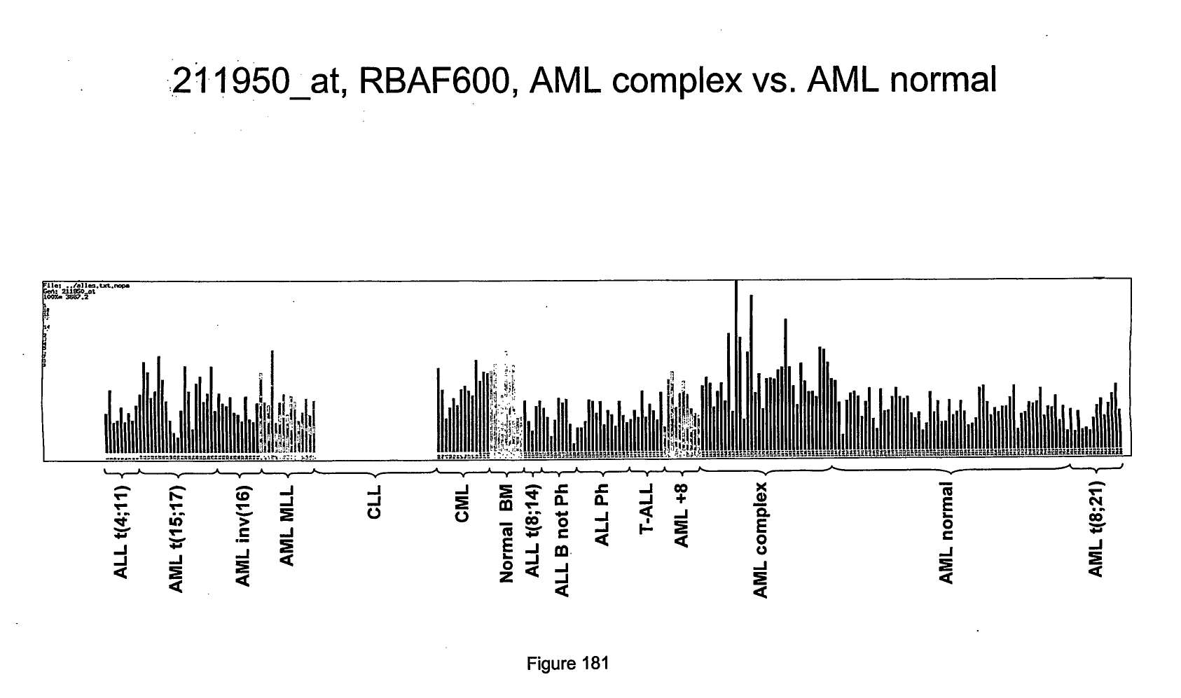

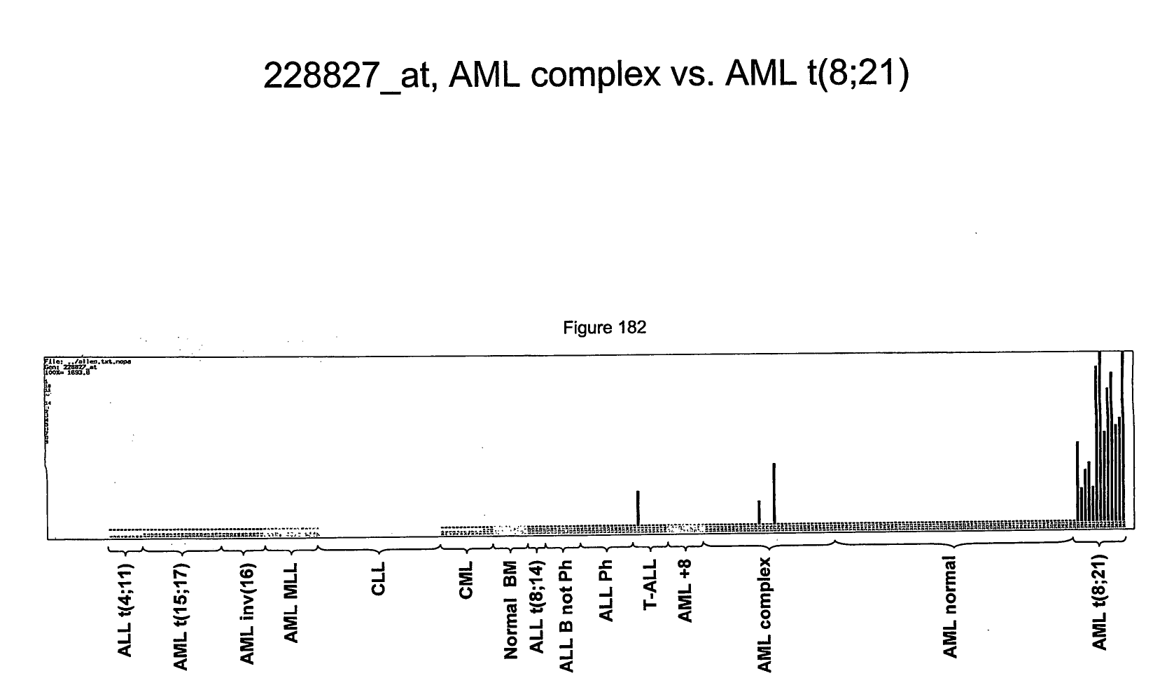

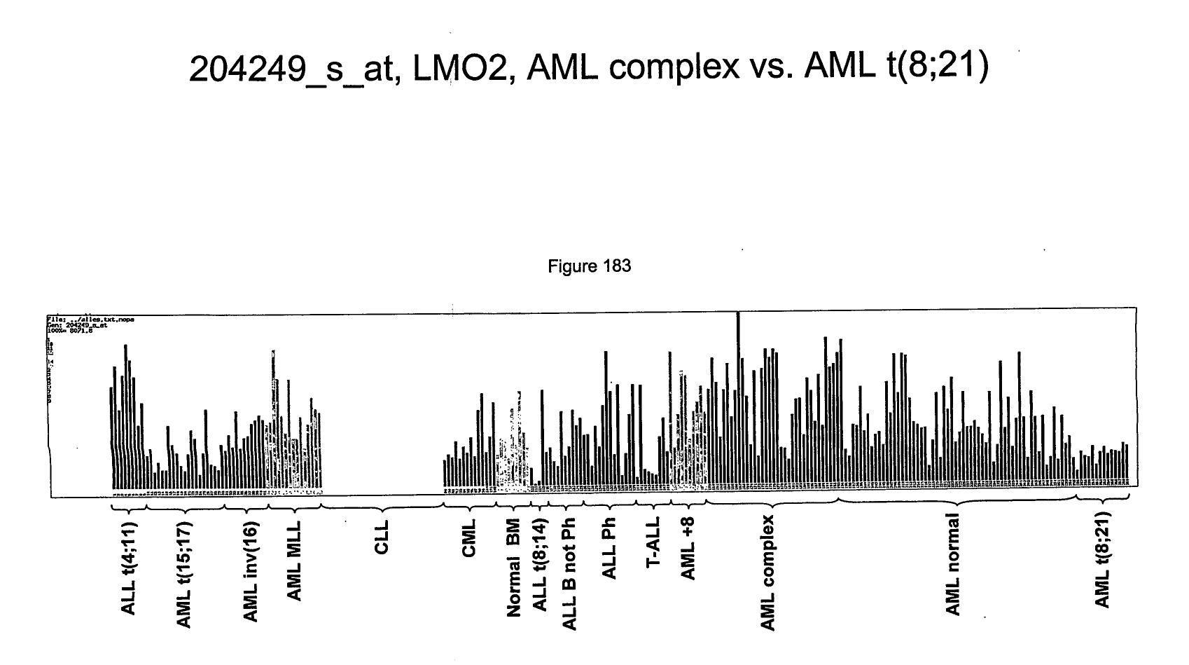

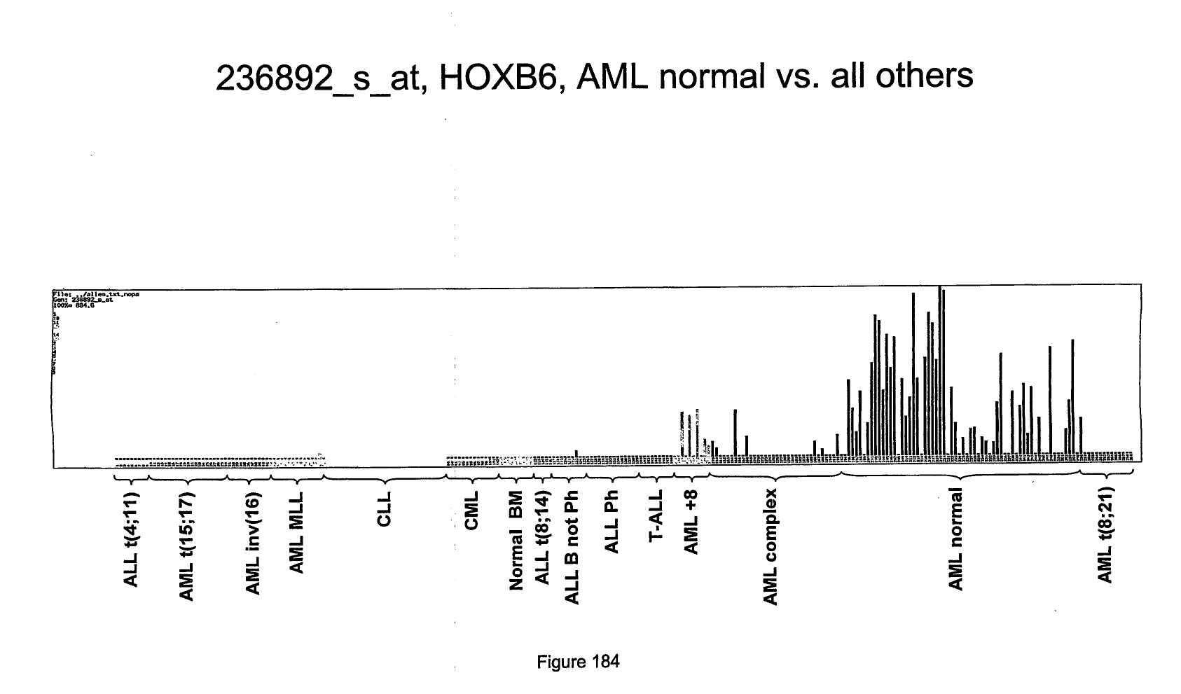

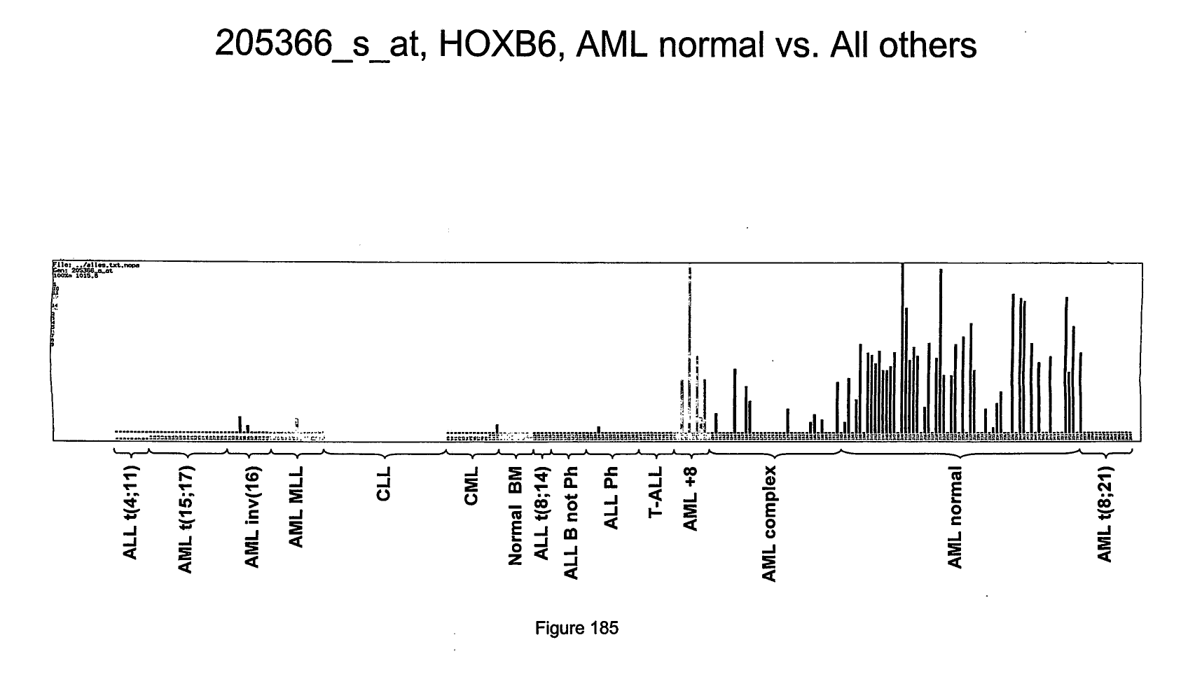

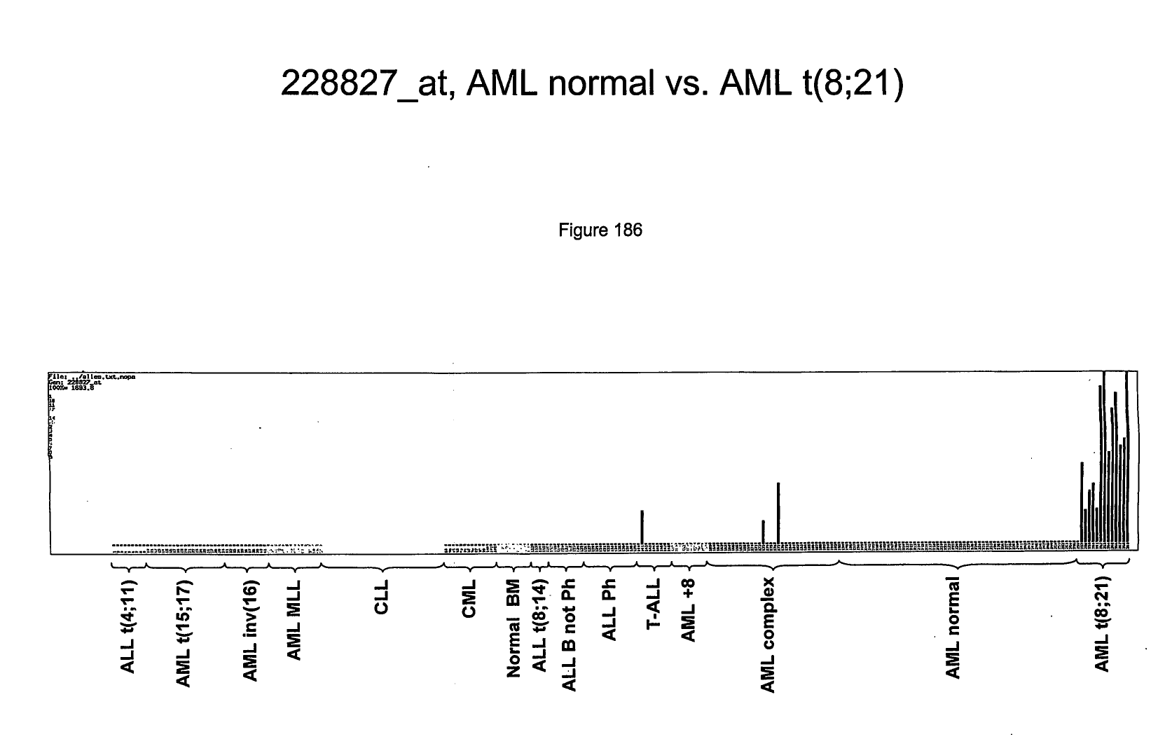

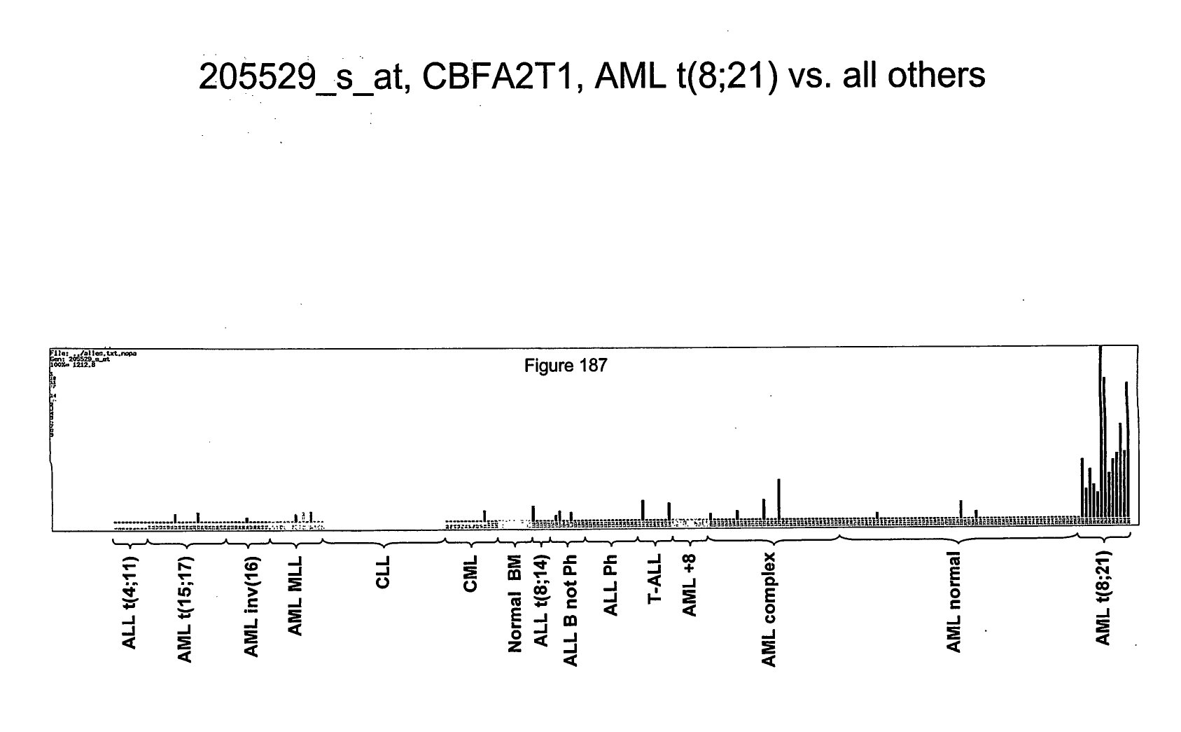

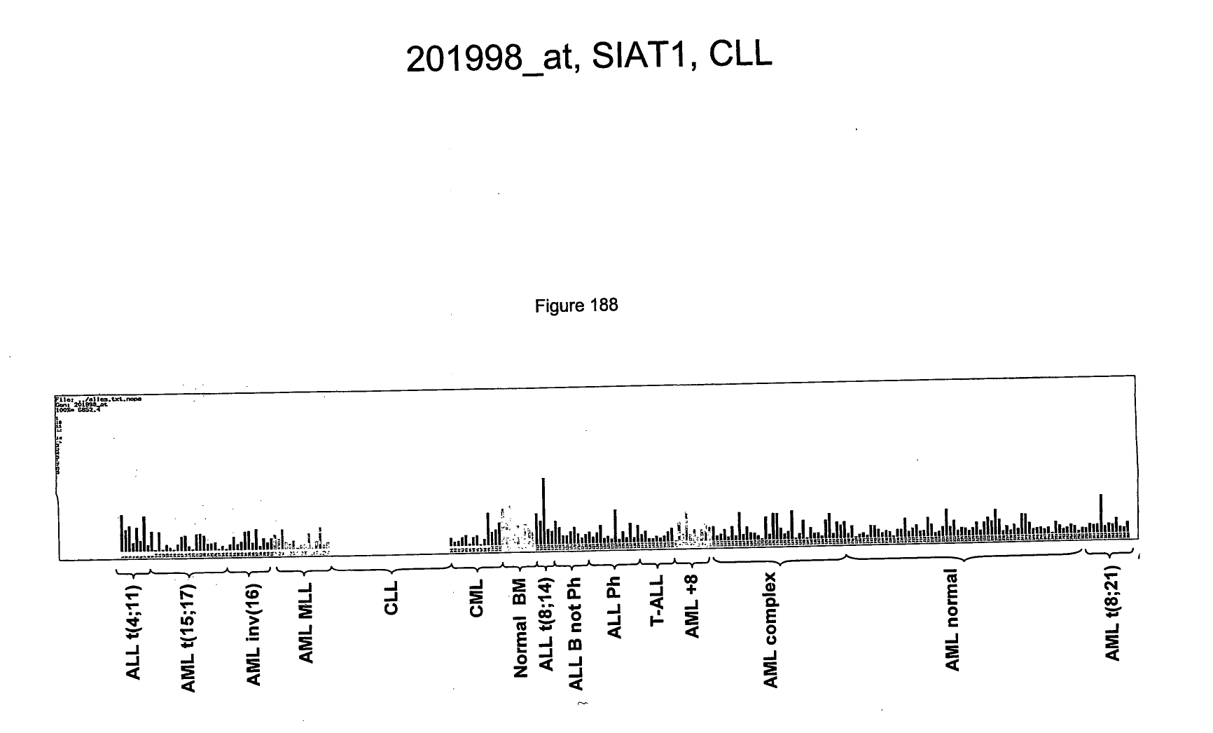

A set of 20 top-ranked genes, which provided both optimal classification accuracy and highest prediction strength for all pairwise (all pairs) and one-versus-all comparisons is given as table 29. Within this set of genes, optimal classification accuracy can be obtained with genes marked by asterisks. Gene expression intensities, plotted as bar graphs are given in FIGS. 24 to 188. Genes are depicted as unique Affymetrix identifier (for example 201497_x_at) and, where available, approved HGNC symbols (HUGO Gene Nomenclature Committee). More detailed, the complete annotation and sequence information about this set of genes is listed in tables 43a,b.

In total 269 cases with leukemia or normal bone marrow (BM) were analyzed. 248 of 269 (92.2%) cases were assigned to the correct leukemia type in all pairwise comparisons (table 28 b). The sensitivity indicated for each subgroup indicates the percentage of cases of the specific subgroup identified correctly in all pairwise comparisons (range 60% to 100%). The specificity indicates for each subgroup the percentage of correct assignments to this subgroup (range 85.3% to 100%).

In total 3766 individual assignments of leukemia and normal bone marrow were analyzed. 3745 of 3766 assignments (99.4%) were correct (table 28c). The sensitivity indicated for each subgroup indicates the percentage of correct assignments for cases of the specific subgroup in pairwise comparisons. (range 97.1% to 100%). The specificity indicates for each subgroup the percentage of correct assignments to this subgroup (range 98.4% to 100%).

In a second approach significant genes were identified according to Westfall & Young. Table 30 represents all genes found to be significant after p-value adjustment. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

Furthermore, we provide information about genes which were found to be rated significant independently by both methodologies (Table 30). Top-significant genes according to the method of example 3 are marked by asterisks. Genes which were included in any of the top-20 lists are marked by positive signs.

In addition, selected gene profiles were chosen to demonstrate their capability of discriminating different leukemia types, subtypes and normal bone marrow, respectively. Gene expression profiles were generated by means of PERL-programs, evaluated and plotted as bar graphs. Each of the analyzed groups are accordingly outlined. The following genes were selected and are given as FIGS. 189 to 233:

| GeneID | gene symbol | feature |
|---|---|---|
| 201162_at | IGFBP7 | CLL low |
| 201163_s_at | IGFBP7 | CLL low |
| 201362_at | NS1-BP | CML high |
| 201496_x_at | MYH11 | AML inv(16) high |
| 201497_x_at | MYH11 | AML inv(16) high |
| 201998_at | SIAT1 | CLL high |
| 202095_s_at | BIRC5 | CLL low |
| 203074_at | ANXA8 | AML t(15; 17) high |
| 204150_at | STAB1 | AML t(15; 17) high |
| 204511_at | KIAA0793 | CLL high |
| 205528_s_at | CBFA2T1 | AML t(8; 21) high |
| 205529_s_at | CBFA2T1 | AML t(8; 21) high |
| 205805_s_at | ROR1 | CLL high |
| 206940_s_at | POU4F1 | AML t(8; 21) high |
| 207819_s_at | ABCB4 | CLL high |
| 208091_s_at | DKFZP564K0822 | CLL high |
| 208456_s_at | RRAS2 | CLL high |
| 209061_at | NCOA3 | CLL high |
| 209101_at | CTGF | ALL t(4; 11) high, |
| | | ALL Ph high, T- |
| | | ALL high |
| 209374_s_at | IGHM | CLL high |
| 209616_s_at | CES1 | AML MLL high |
| 210997_at | HGF | AML t(15; 17) high |
| 212285_s_at | AGRN | AML t(15; 17) high |
| 213539_at | CD3D | T-ALL high |
| 214450_at | CTSW | AML t(15; 17) high |
| 215925_s_at | | ALL t(4; 11) high |
| 218223_s_at | LOC51177 | CML low |
| 222166_at | | AML +8 high |
| 224520_s_at | MGC13168 | ALL t(8; 14) high |
| 224794_s_at | LOC51148 | AML t(15; 17) high |
| 225660_at | SEMA6A | ALL B not Ph |
| | | high, ALL Ph high |
| 226496_at | Homo sapiens, Similar to | ALL high, CLL |
| | hypothetical protein FLJ22611, | |
| | clone MGC: 24716 | high |
| | IMAGE: 4277726, | |
| | mRNA, complete cds | |
| 228827_at | Homo sapiens clone 25023 | AML t(8; 21) high |
| | mRNA sequence | |
| 228904_at | ESTs | AML normal high, |
| | | AML +8 high, AML |
| | | complex high |
| 236301_at | Homo sapiens, clone | CLL high |
| | IMAGE: 3866403, mRNA | |
| 236892_s_at | HOXB6 | AML normal high, |
| | | AML +8 high, AML |
| | | complex high |
| 239214_at | ESTs | ALL t(4; 11) high |
| 239393_at | ESTs | ALL t(4; 11) high |
| 239791_at | HOXB6 | AML normal high, |
| | | AML +8 high |
| 240581_at | ESTs | ALL t(4; 11) high |
| 241464_s_at | ESTs | AML MLL high, |
| | | AML normal high, |
| | | AML +8 high, AML |
| | | complex high |
| 241525_at | ESTs | AML inv(16) high |
| 243362_s_at | LEF1 | ALL high, CLL |
| | | high |
| 36566_at | CTNS | T-ALL low |
| 38487_at | FLJ12442 | AML t(15; 17) high |

Generally, chromosomal aberrations are strongly associated with morphological characteristics. However, there are two chromosomal aberrations which are observed in both myeloid and lymphatic neoplasms, i.e. t(11q23)/MLL and the t(9;22). The t(9;22) occurs in ALL (ALL Ph) and CML, and t(11q23)/MLL is observed in ALL (ALL t(4;11)) and AML (AML MLL), respectively. Analysing gene expression signatures of both t(9;22) positive ALL and CML we identified genes, which allowed correct lineage assignments (table 29). In addition, our results indicate that the distinct expression signatures are also sufficient for correct assignments of the t(11q23)/MLL positive leukemias either to ALL or to AML (table 29). Thus, in both scenarios lineage assignment (lymphoid or myeloid), and even subtype classification can be accomplished based on the methods and markers described herein, despite of the fact that e.g., in the above-noted t(11q23) and t(9;22) chromosomal aberrations, the same chromosomal aberration is associated with different kinds of leukemia.

### Example 7

### Results V: Analysis of 5 ALL subtypes Defined by Genetics and Immunophenotype

Here we analyzed in 5 distinct ALL subtypes. We applied the described two different statistical methods for identification of genes which allow accurate class assignments to the respective groups.

| | |
|---|---|
| ALL t(4; 11) | (n = 9) |
| ALL t(8; 14) | (n = 4) |
| ALL B not Ph | (n = 9) |
| ALL Ph | (n = 15) |
| T-ALL | (n = 9) |

First, expression data were analyzed according to example 3, as described hereinabove.

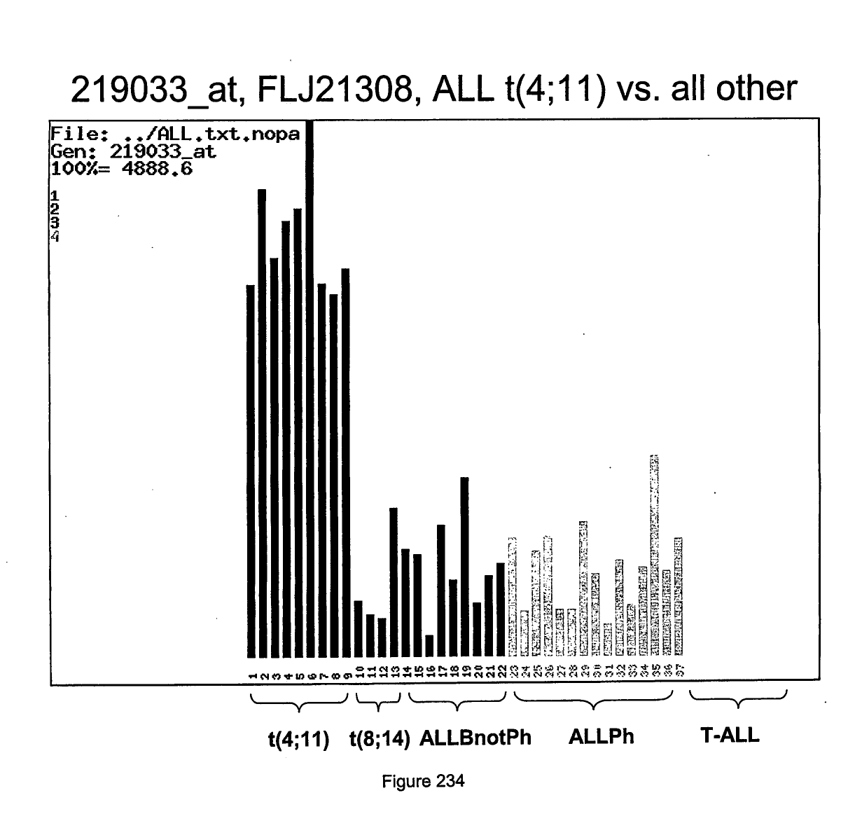

A set of 20 top-ranked genes, which provided both optimal classification accuracy and highest prediction strength for all pairwise (all pairs) and one-versus-all comparisons is given in table 32. Within this set of genes, optimal classification accuracy can be obtained with genes marked by asterisks. Gene expression intensities, plotted as bar graphs are given in FIGS. 234 to 252. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

In total 46 cases of ALL were analyzed. 44 of 46 cases (95.7%) were assigned to the correct ALL subtype in all pairwise comparisons (table 31a). The sensitivity indicated for each subgroup indicates the percentage of cases of the specific subgroup identified correctly in all pairwise comparisons (range 88.9% to 100%). The specificity indicates for each subgroup the percentage of correct assignments to this subgroup (range 88.9% to 100%).

In total 184 individual assignments of ALL were analyzed. 182 of 184 assignments (98.9%) were correct (table 31b). The sensitivity indicated for each subgroup indicates the percentage of correct assignments for cases of the specific subgroup in pairwise comparisons. (range 97.2% to 100%). The specificity indicates for each subgroup the percentage of correct assignments to this subgroup (range 97.2% to 100%).

In a second approach significant genes were identified according to Westfall & Young. Table 33 represents all genes found to be significant after p-value adjustment. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

Furthermore, we provide information about genes which were found to be rated significant independently by both methodologies (Table 33). Top-significant genes according to the method of example 3 hereinabove are marked by asterisks. Genes which were included in any of the top-20 lists are marked by positive signs.

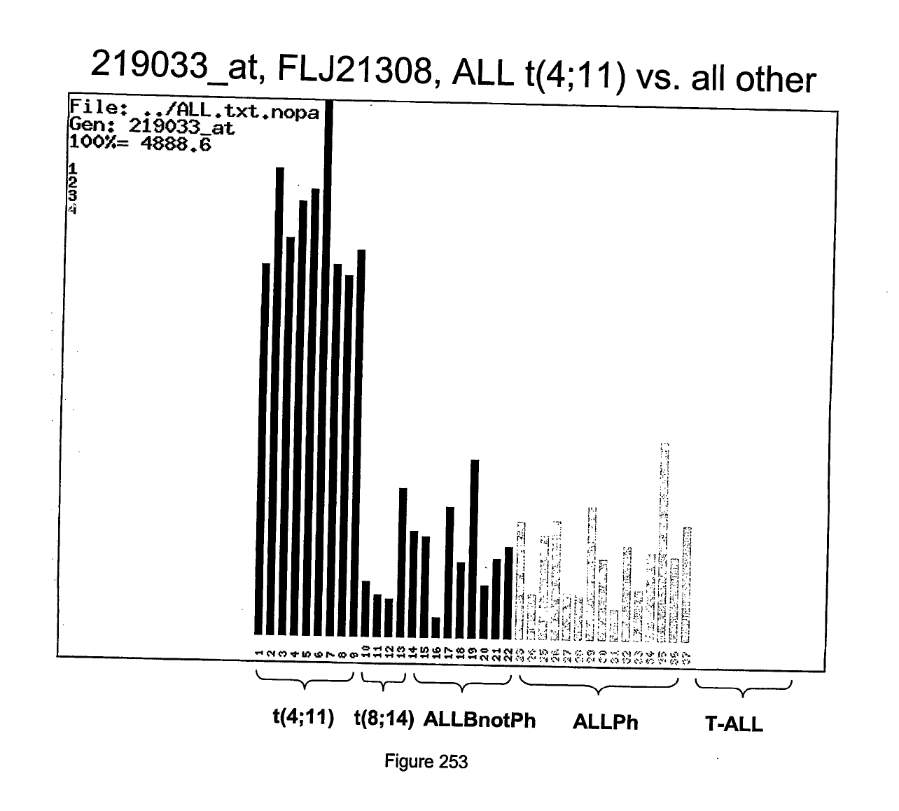

In addition, selected gene profiles were chosen to demonstrate their capability of discriminating different leukemia types, subtypes and normal bone marrow, respectively. Gene expression profiles were generated by means of PERL-programs, evaluated and plotted as bar graphs. Each of the analyzed groups are accordingly outlined. The following genes were selected and are given as FIGS. 253 to 271:

| GeneID | gene symbol | feature |
|---|---|---|
| 201105_at | LGALS1 | ALL t(4; 11) high |
| 204044_at | QPRT | ALL t(4; 11) high |
| 205899_at | CCNA1 | ALL t(4; 11) high |
| 209168_at | GPM6B | ALL t(4; 11) high |
| 213539_at | CD3D | T-ALL high |
| 213894_at | KIAA0960 | ALL t(4; 11) high |
| 215925_s_at | | ALL t(4; 11) high |
| 218224_at | PNMA1 | T-ALL high |
| 219463_at | C20orf103 | ALL t(4; 11) high |
| 219631_at | FLJ12929 | T-ALL high |
| 225563_at | ESTs | ALL t(4; 11) high |
| 225592_at | NRM | ALL t(4; 11) high |
| 228083_at | Homo sapiens mRNA; cDNA | ALL t(4; 11) high |
| | DKFZp434I1216 (from clone | |
| | DKFZp434I1216) | |
| 228988_at | ZNF6 | T-ALL high |
| 235749_at | | ALL t(8; 14) high |
| 242414_at | ESTs | ALL t(4; 11) high |
| 243756_at | ESTs | ALL t(4; 11) high |

### Example 7

### Results VI: Analysis of 8 AML Subtypes

Here we analyzed in total 8 distinct AML subtypes. We applied the described two different statistical methods for identification of genes which allow accurate class assignments to the respective groups.

| | |
|---|---|
| trisomy 8 | (n = 10) |
| other aberrant | (n = 5) |
| complex | (n = 36) |
| normal | (n = 62) |
| t(8; 21) | (n = 13) |
| t(15; 17) | (n = 20) |
| inv(16) | (n = 12) |
| MLL | (n = 15) |

First, expression data were analyzed according to example 3 as described hereinabove.

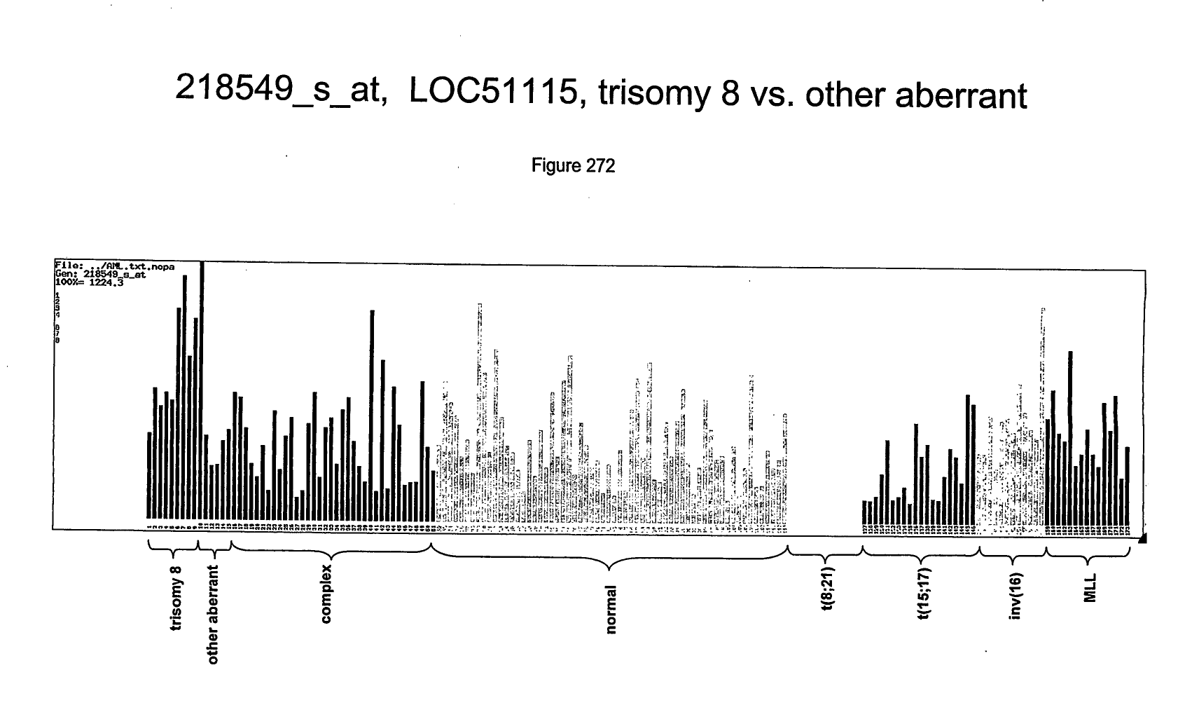

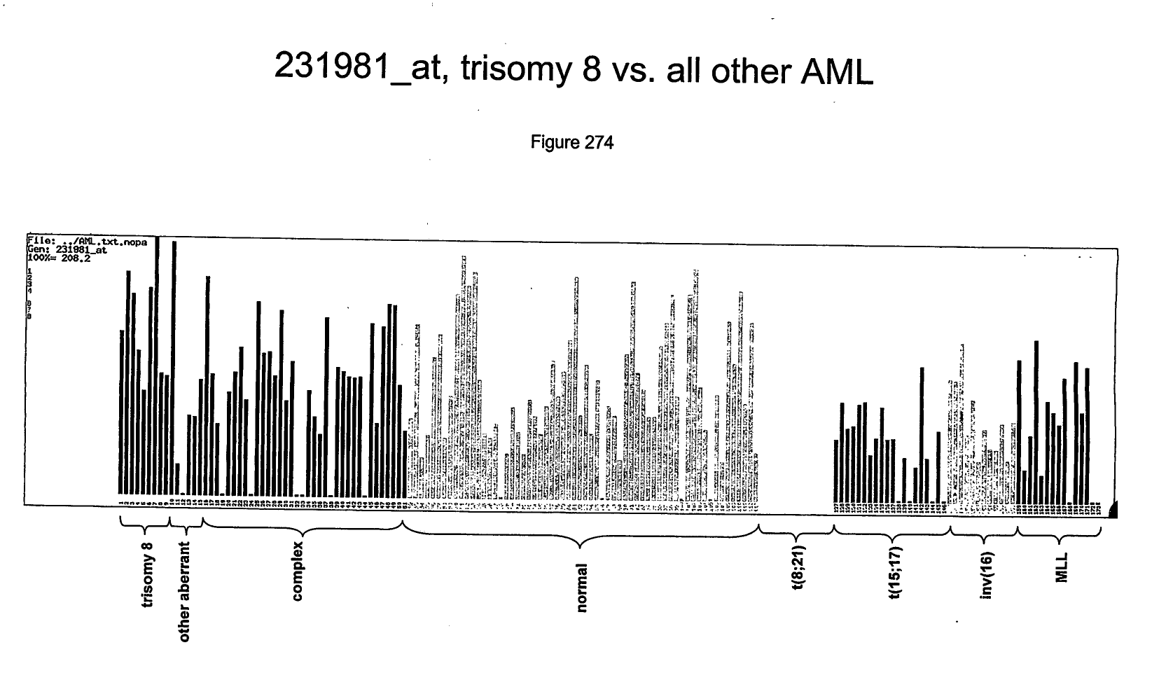

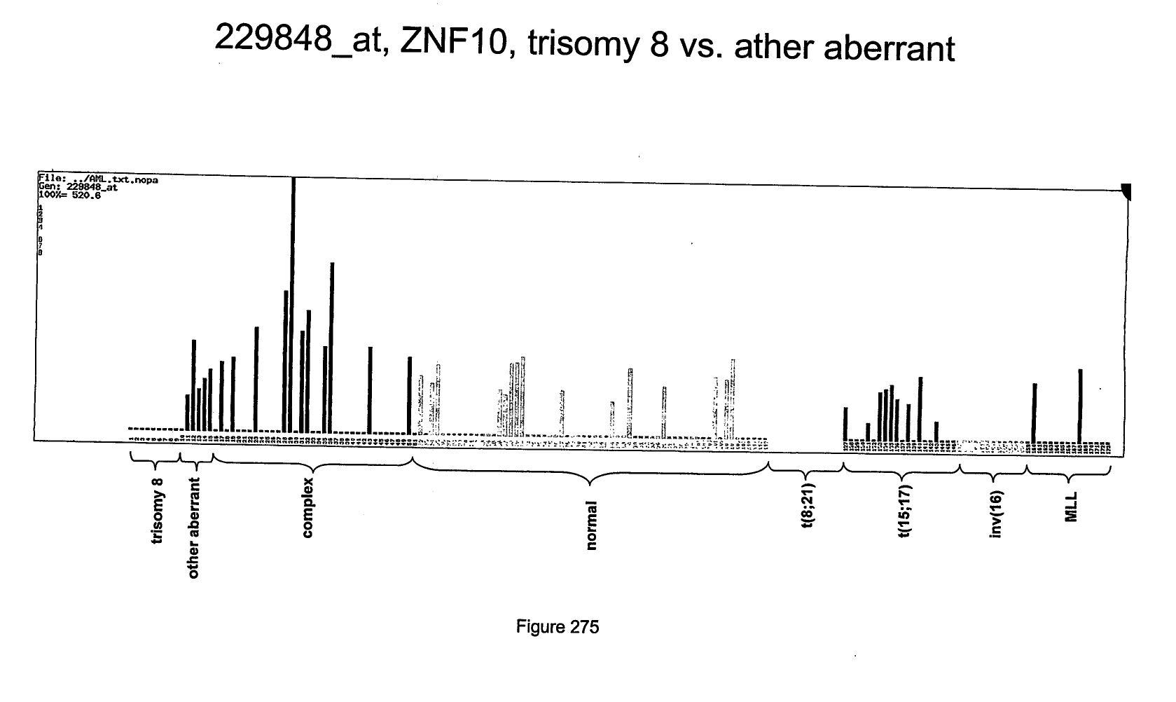

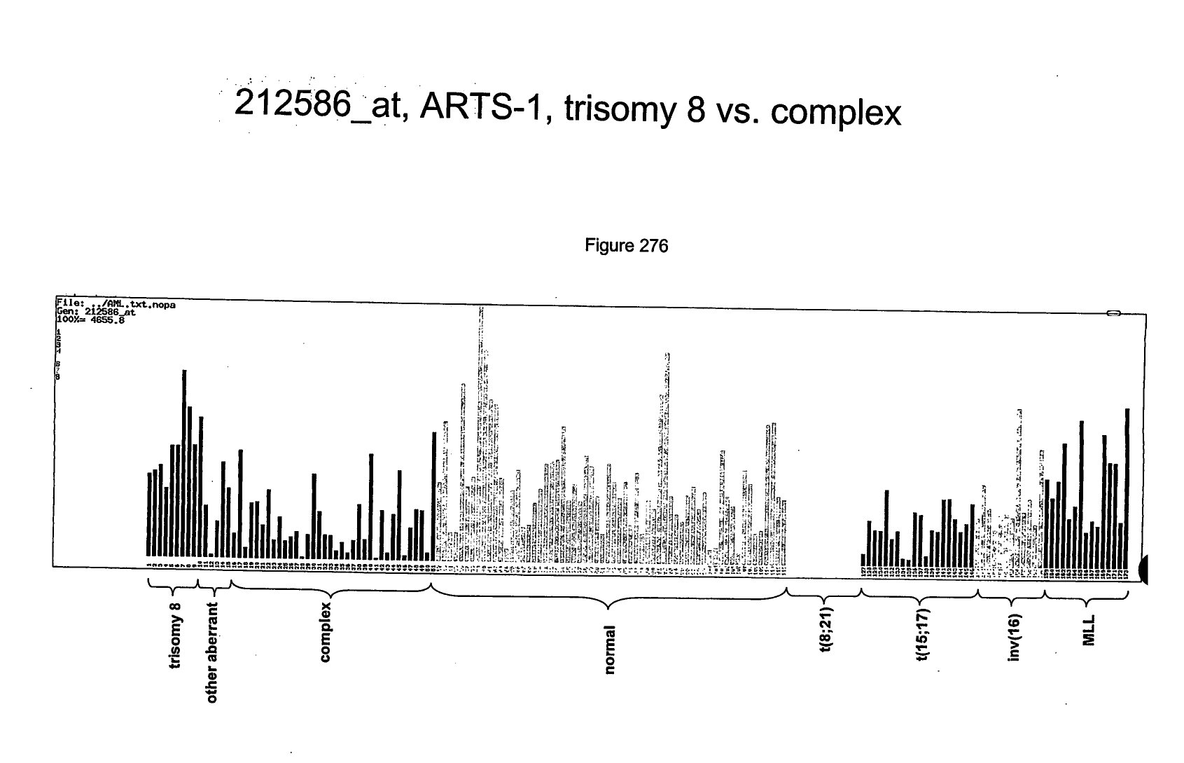

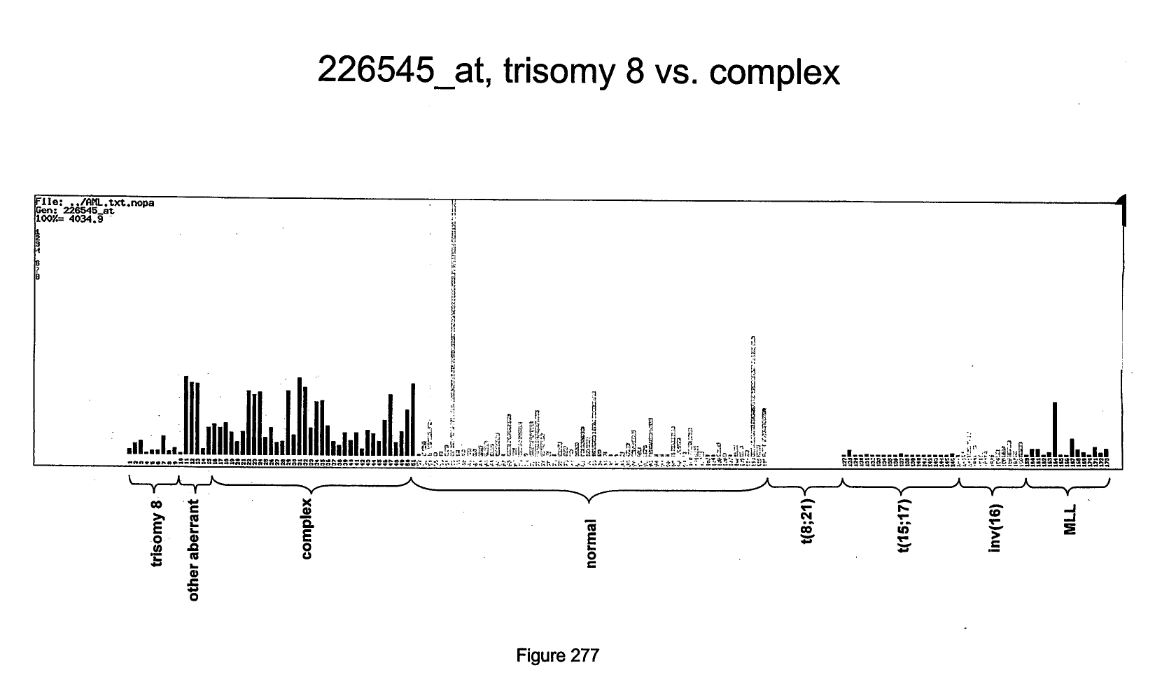

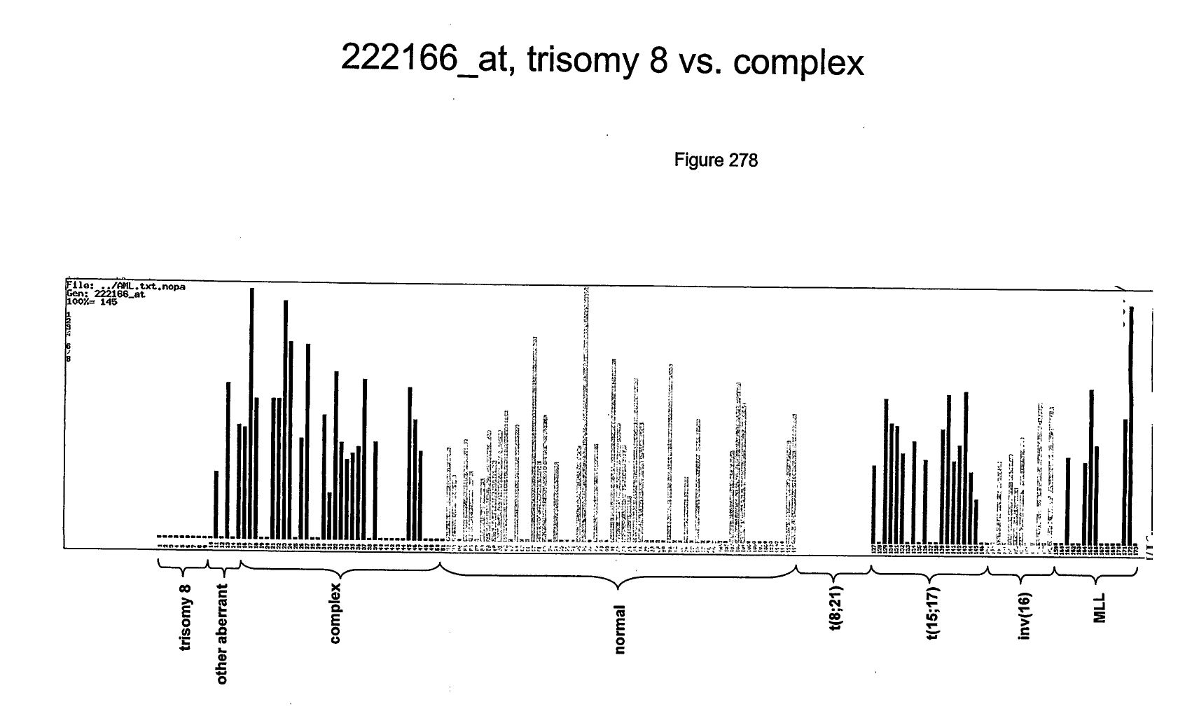

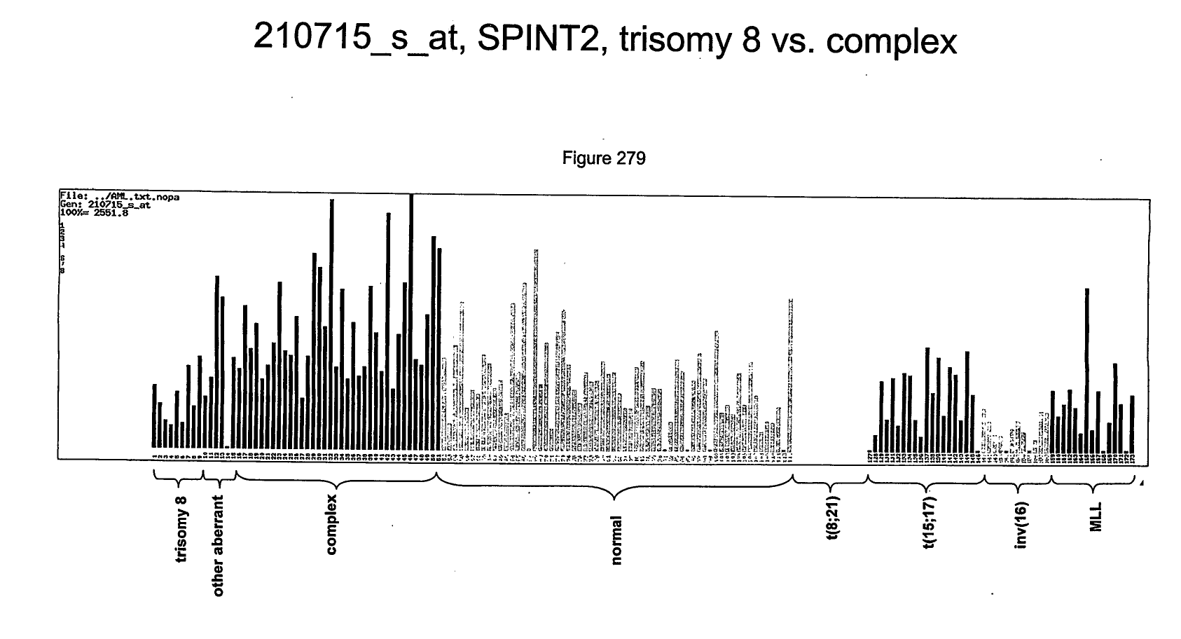

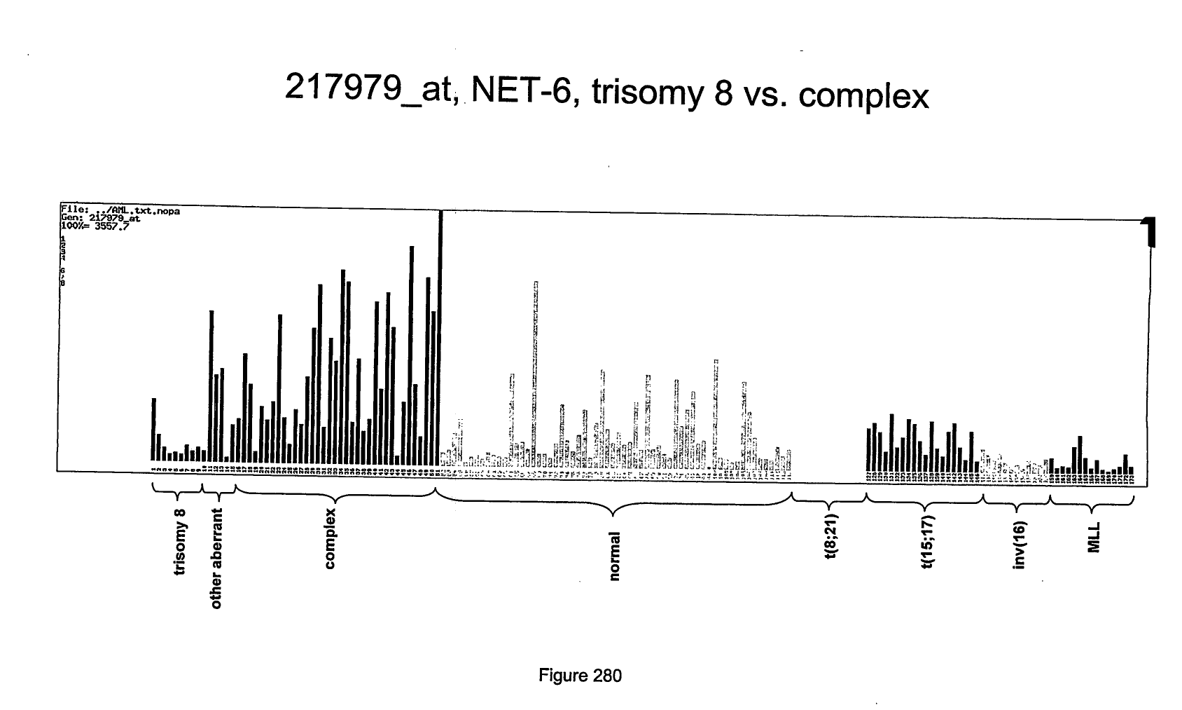

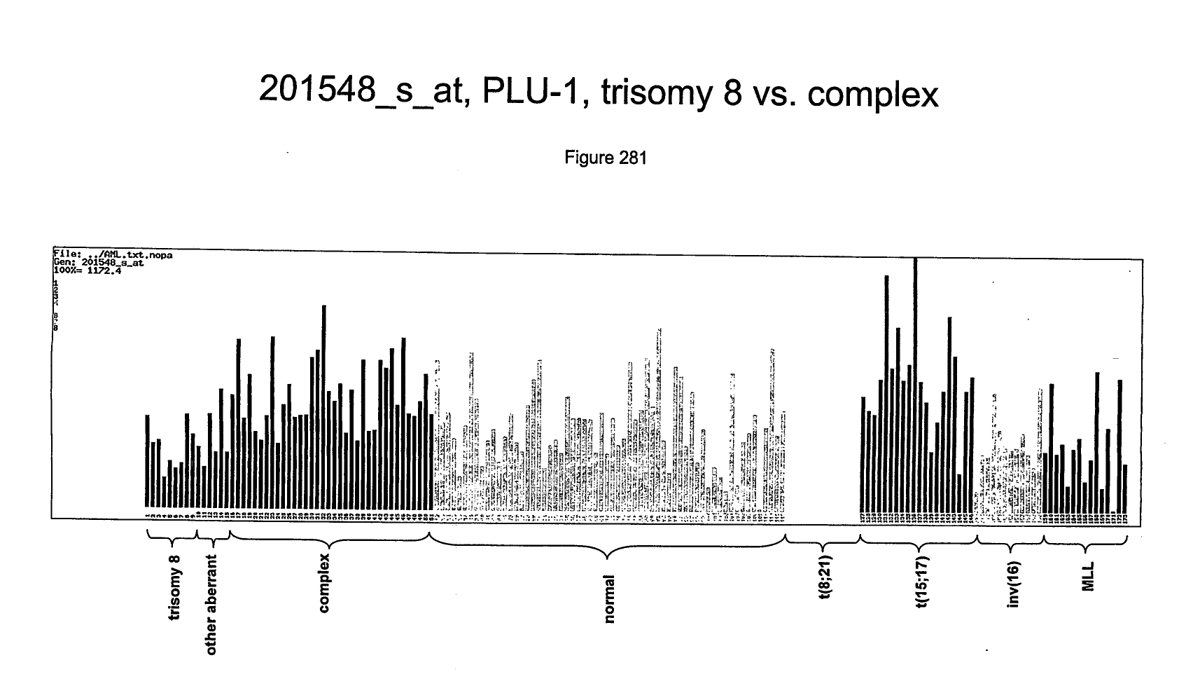

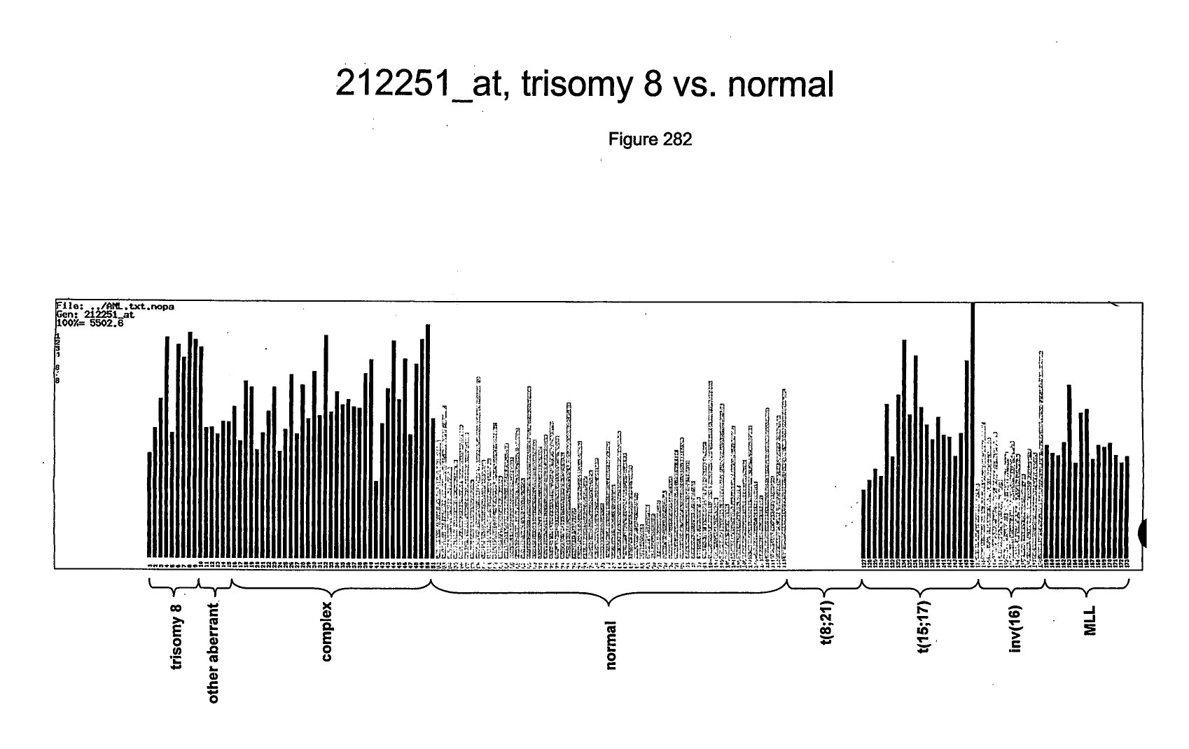

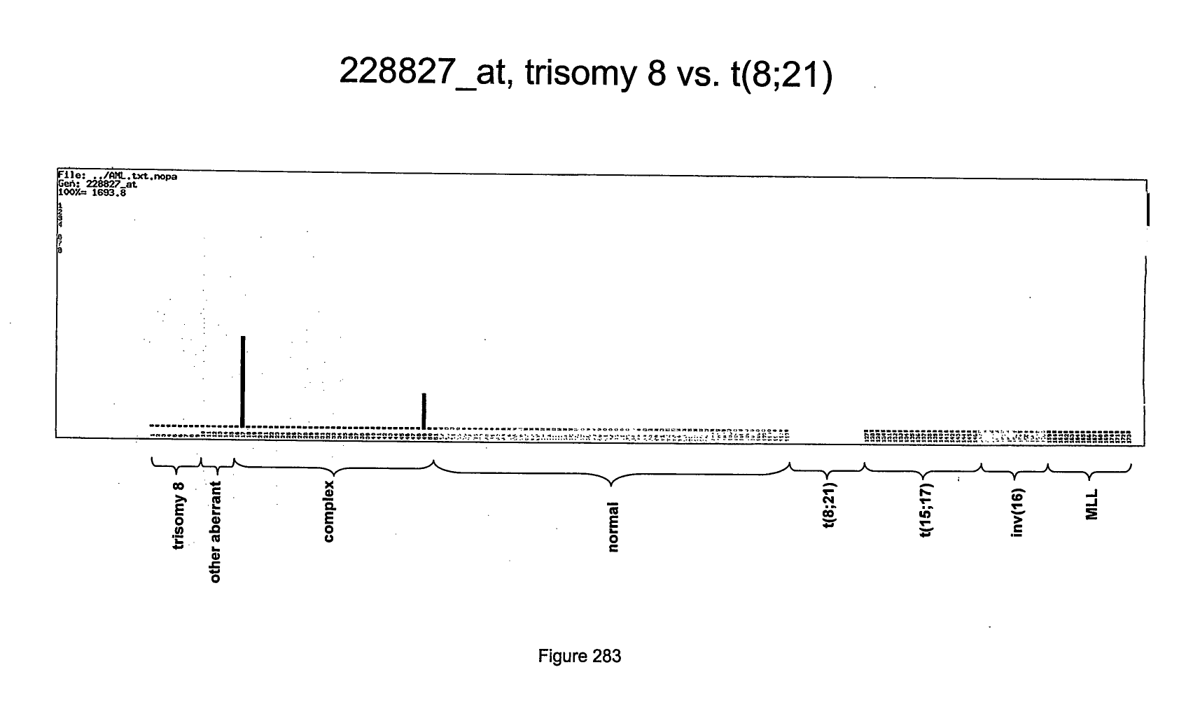

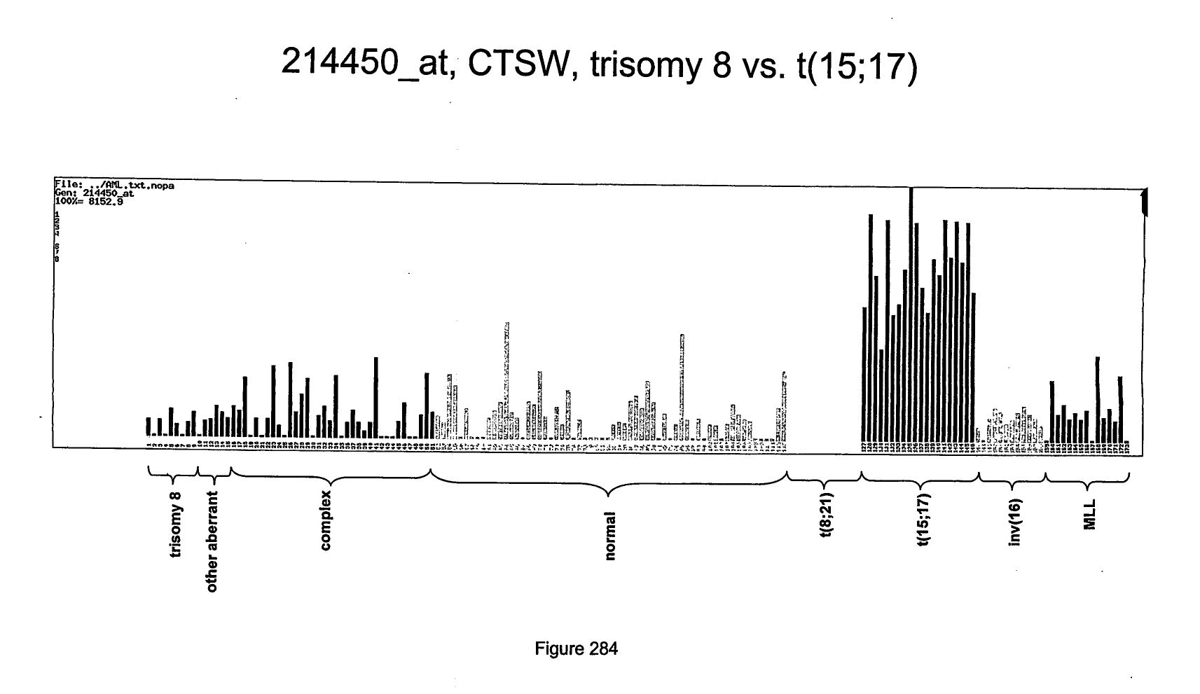

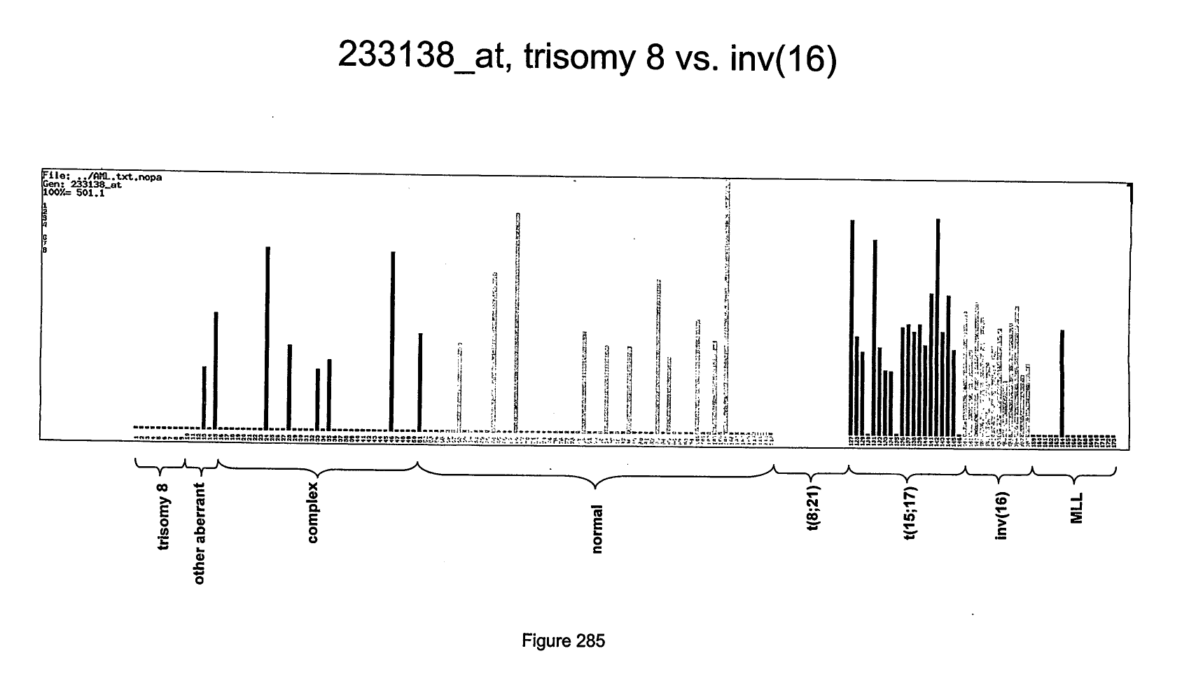

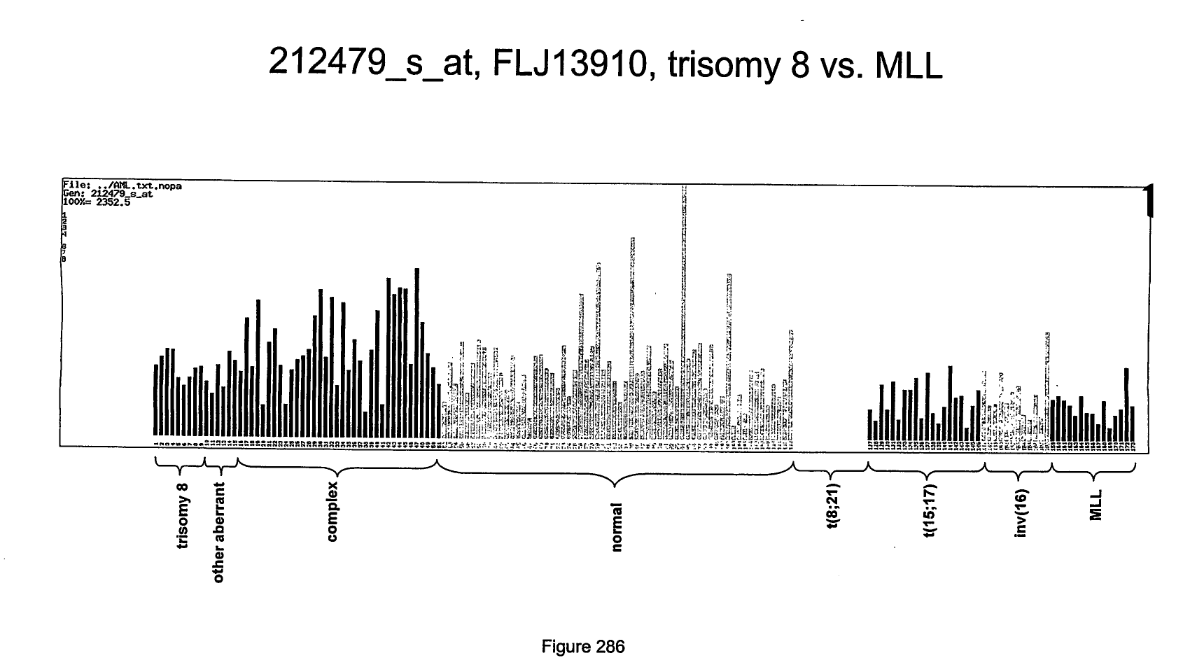

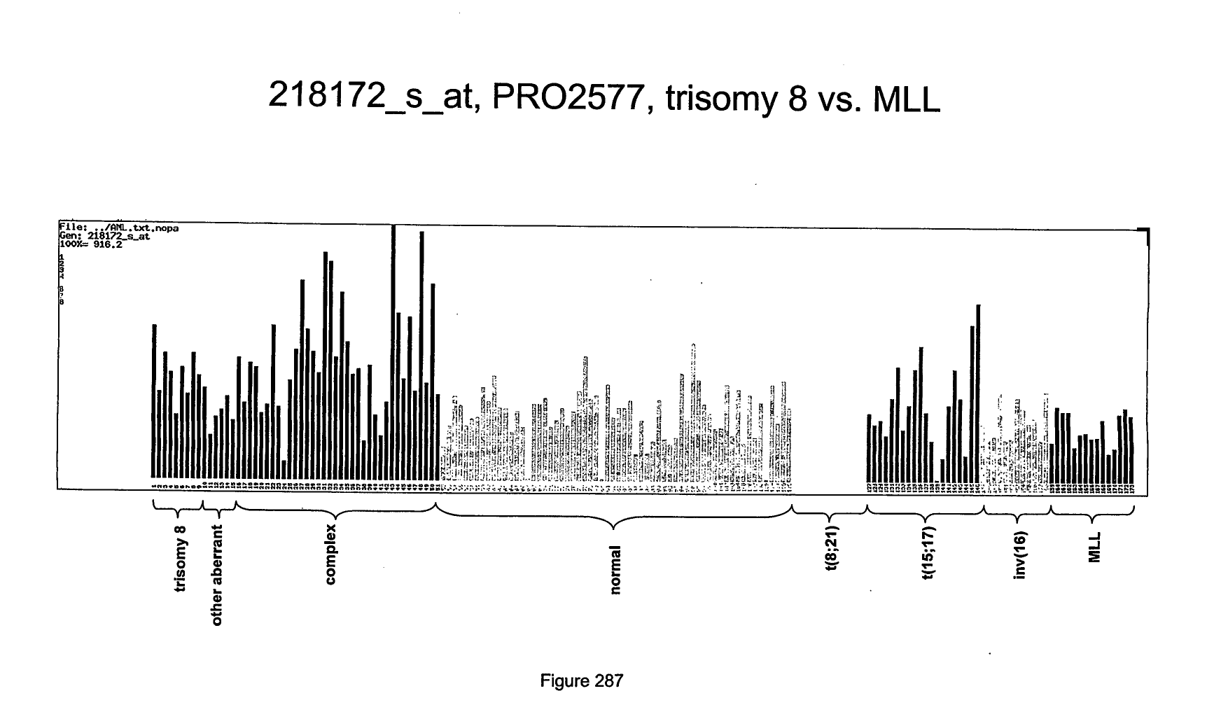

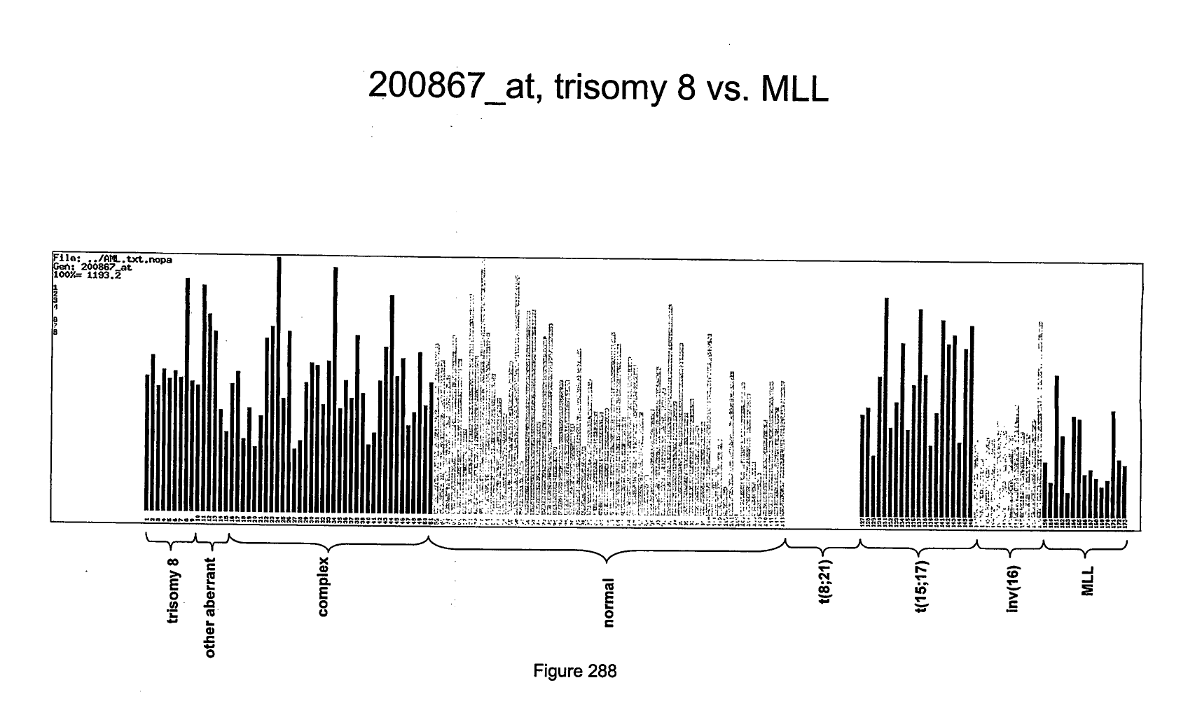

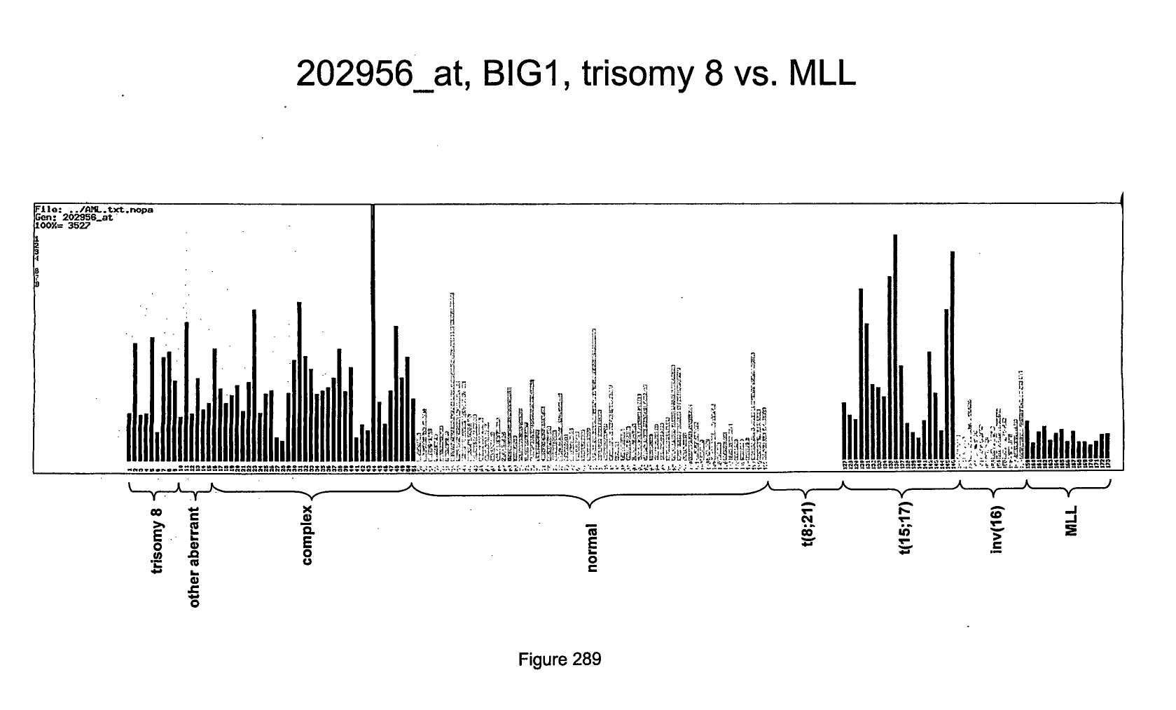

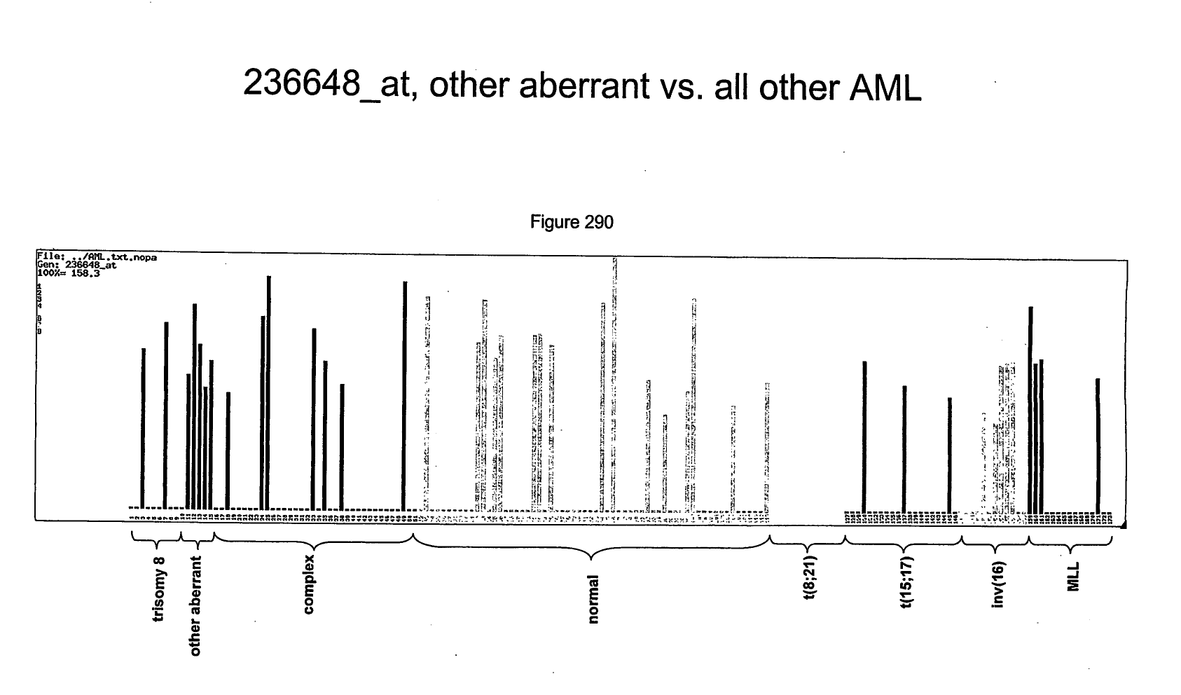

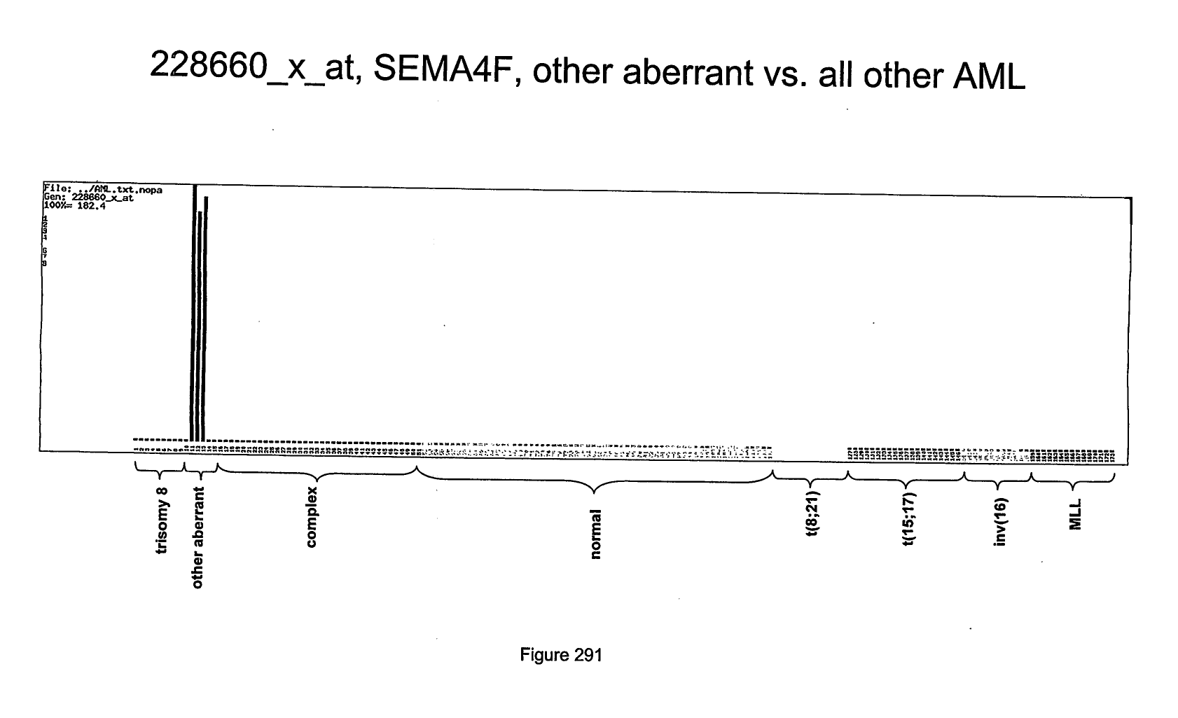

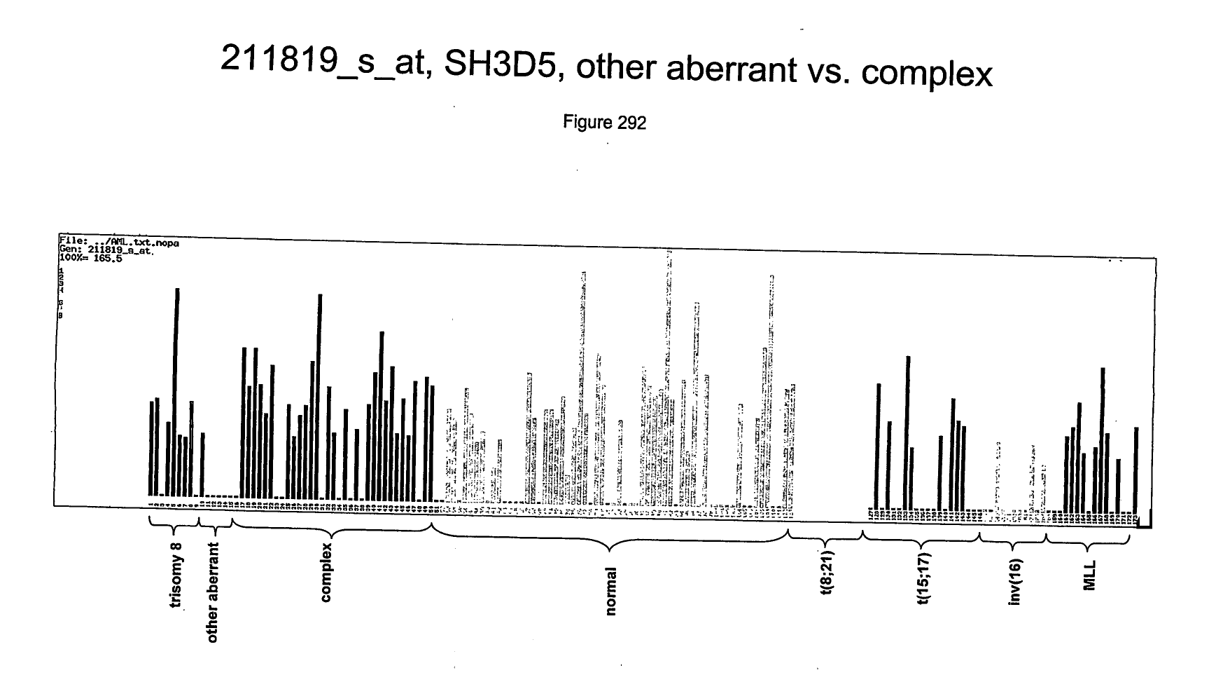

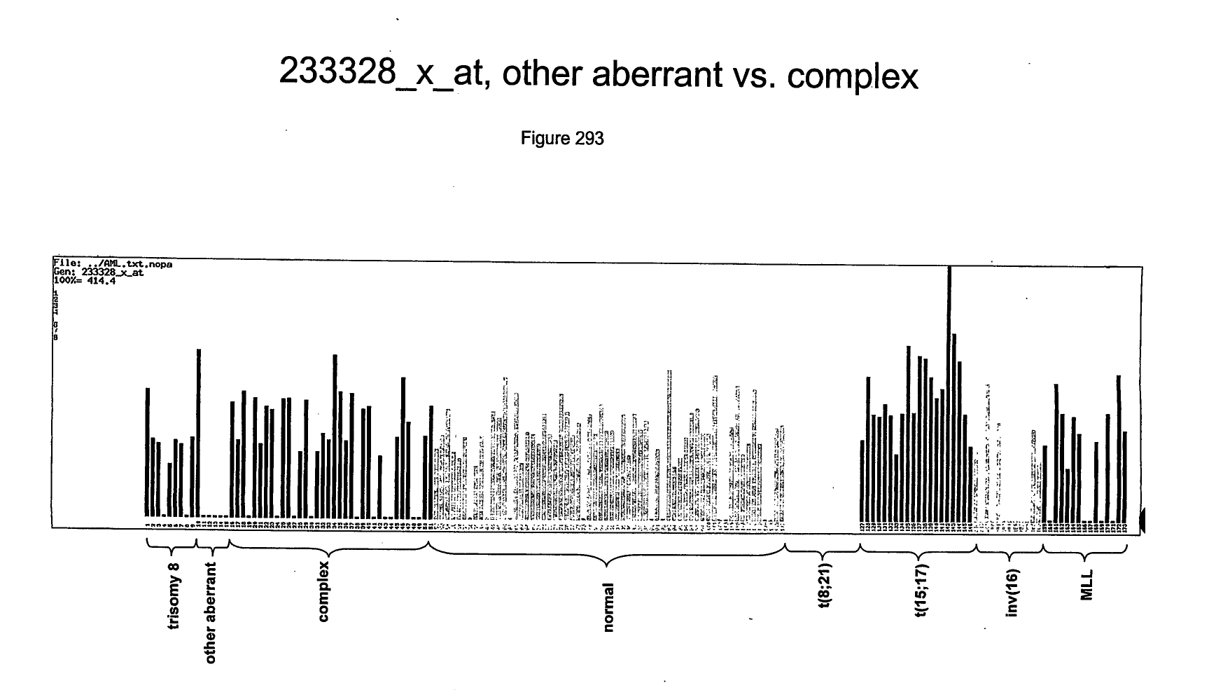

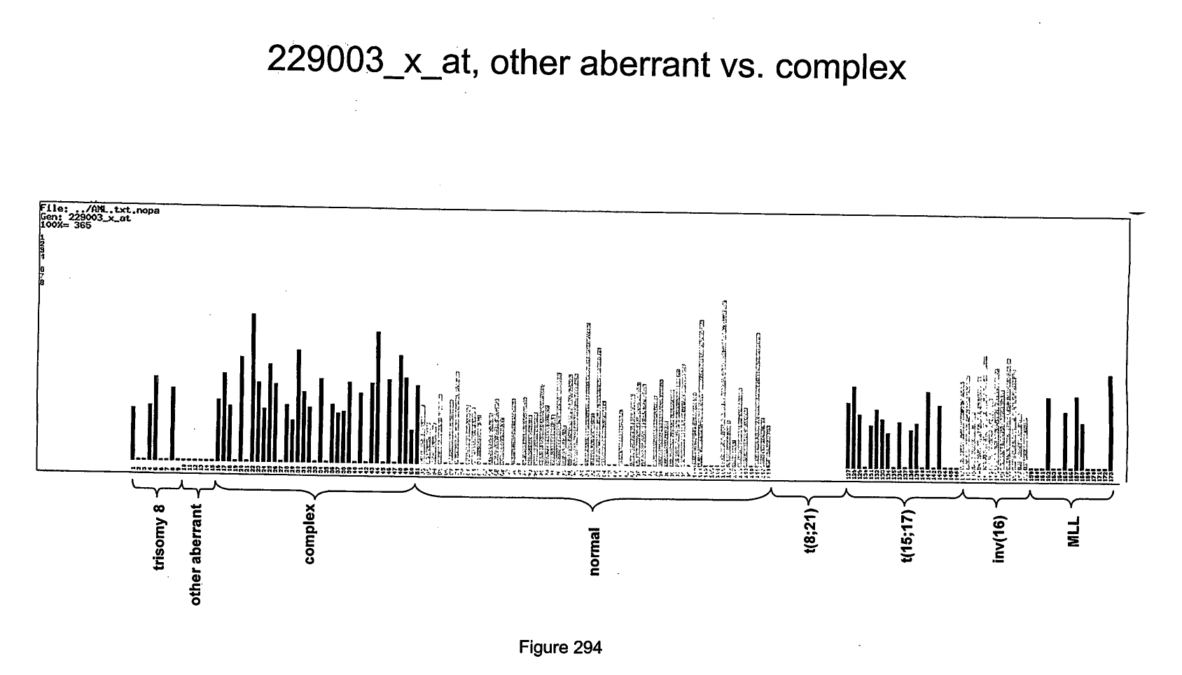

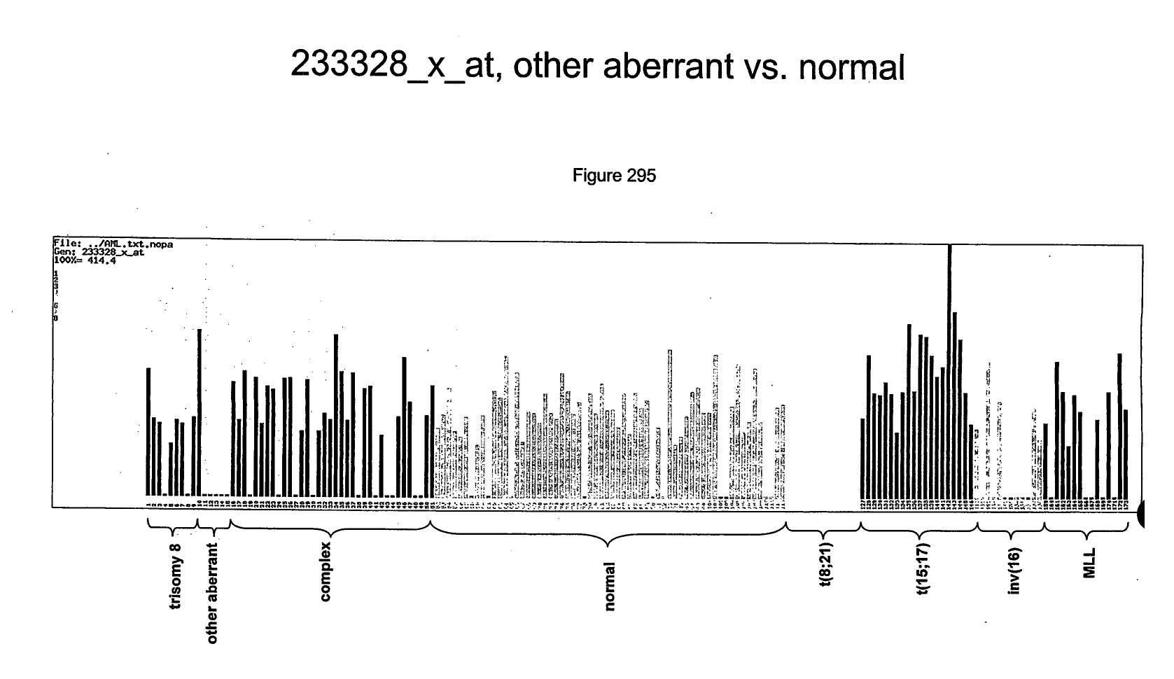

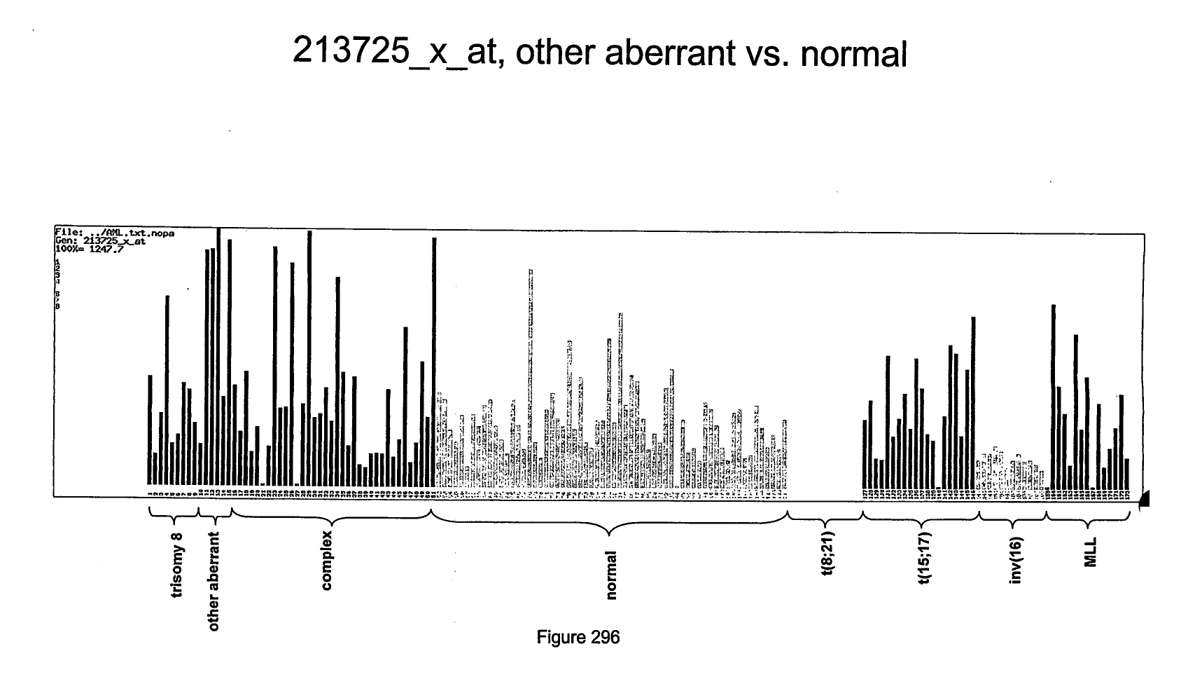

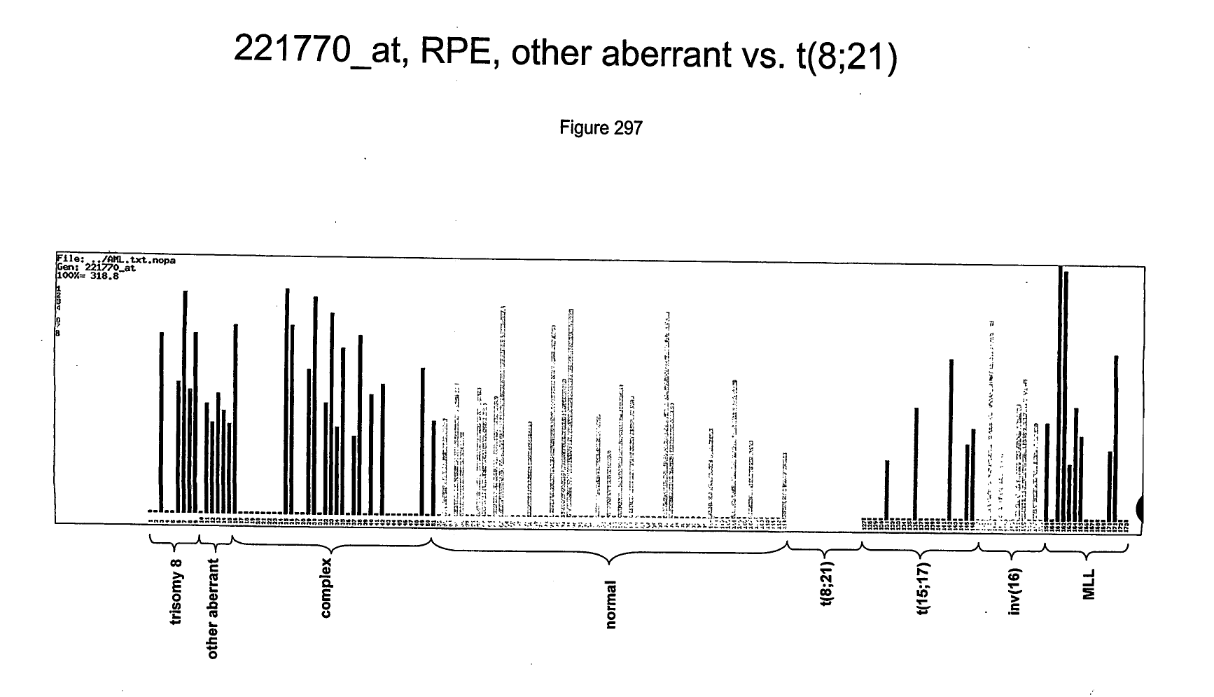

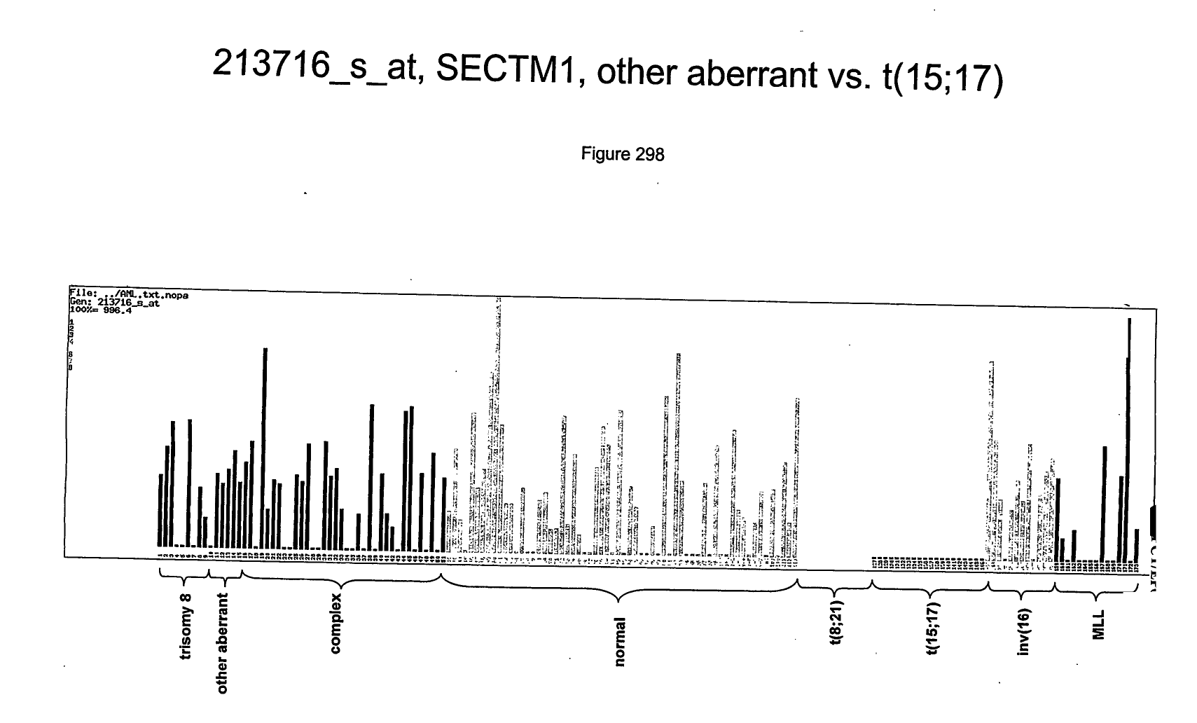

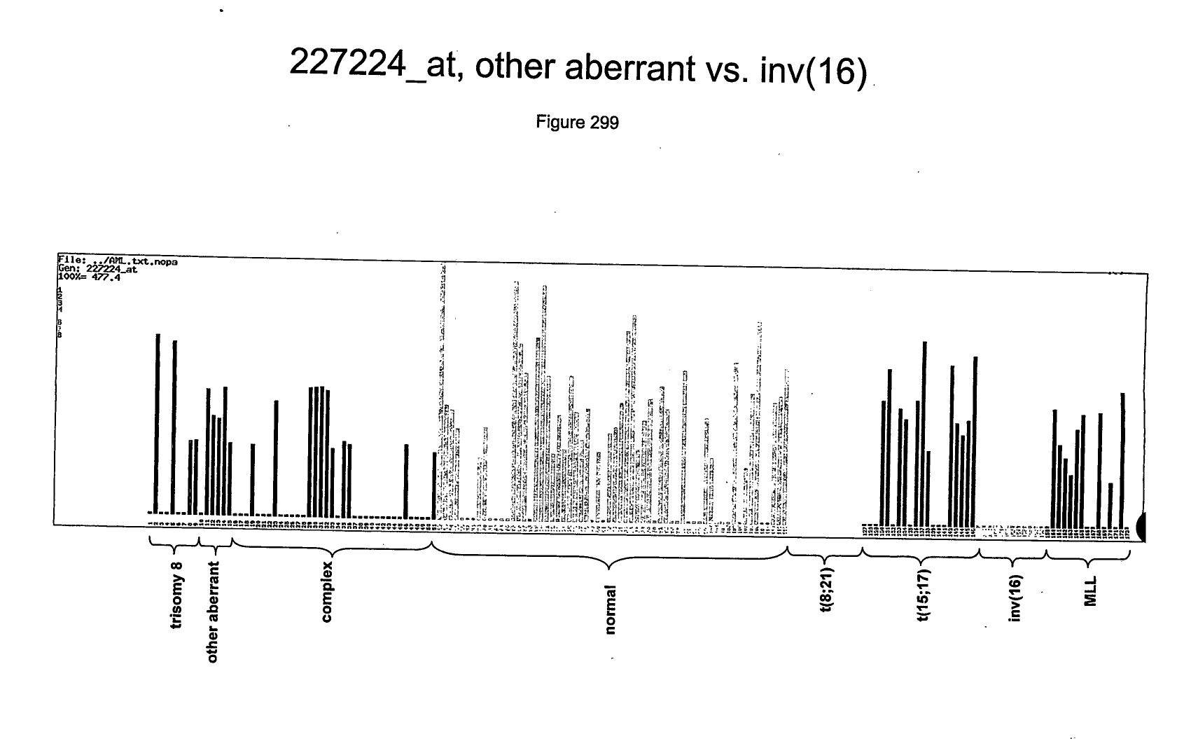

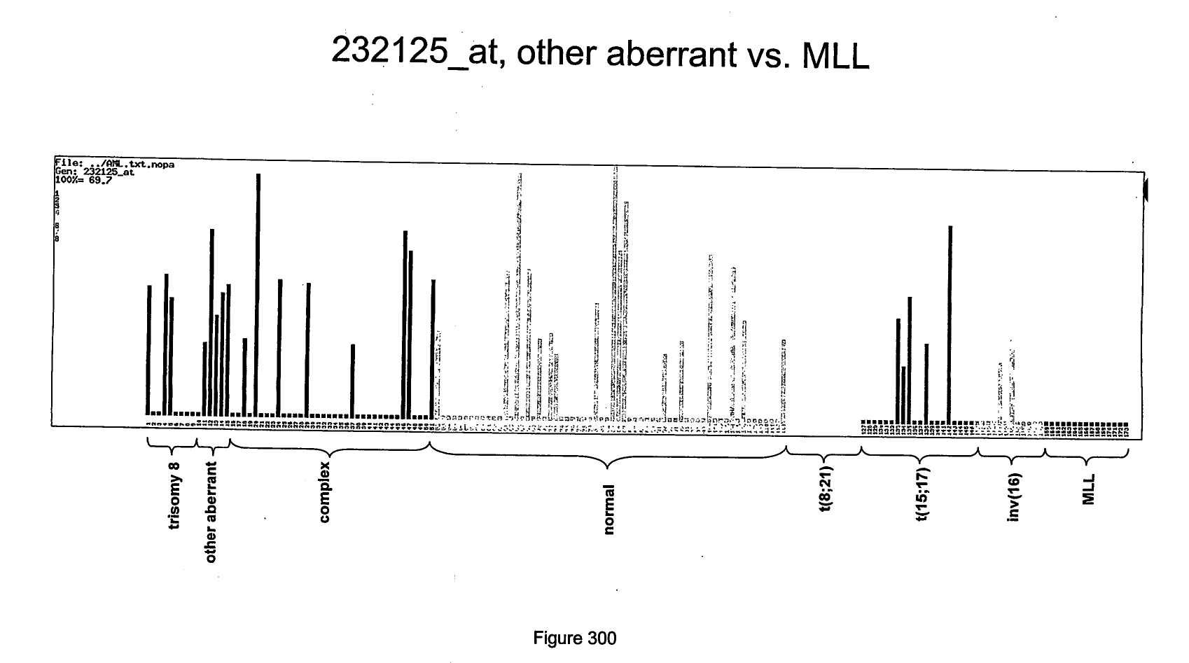

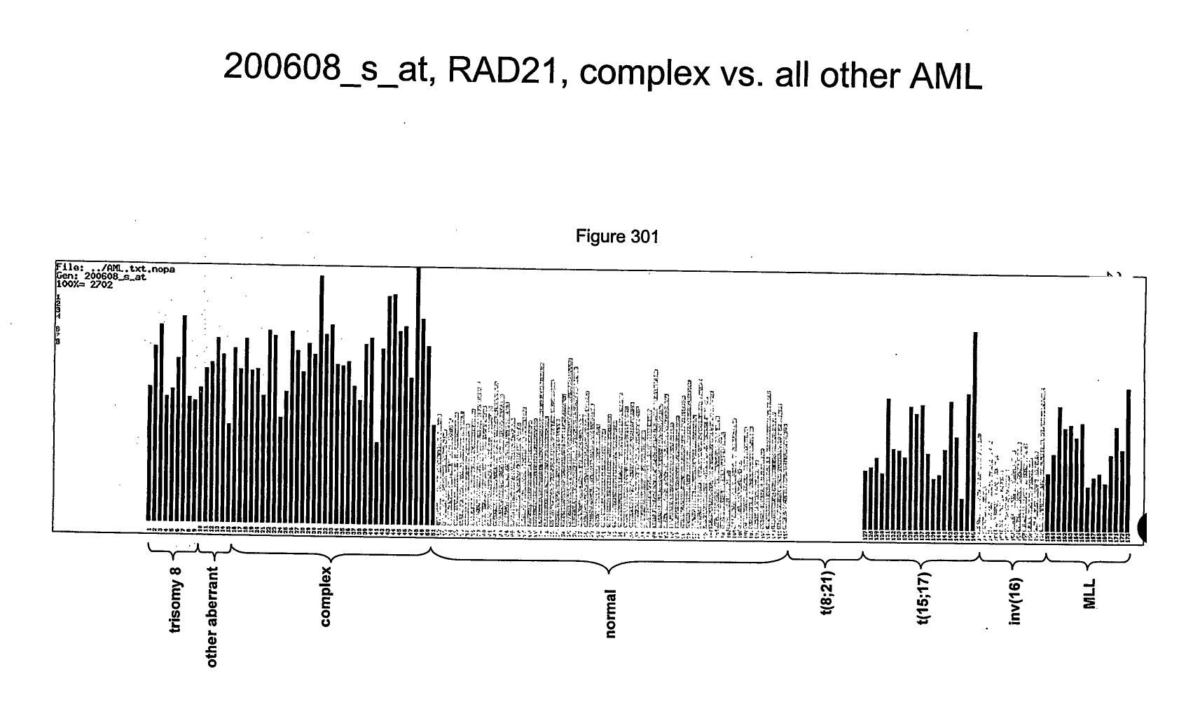

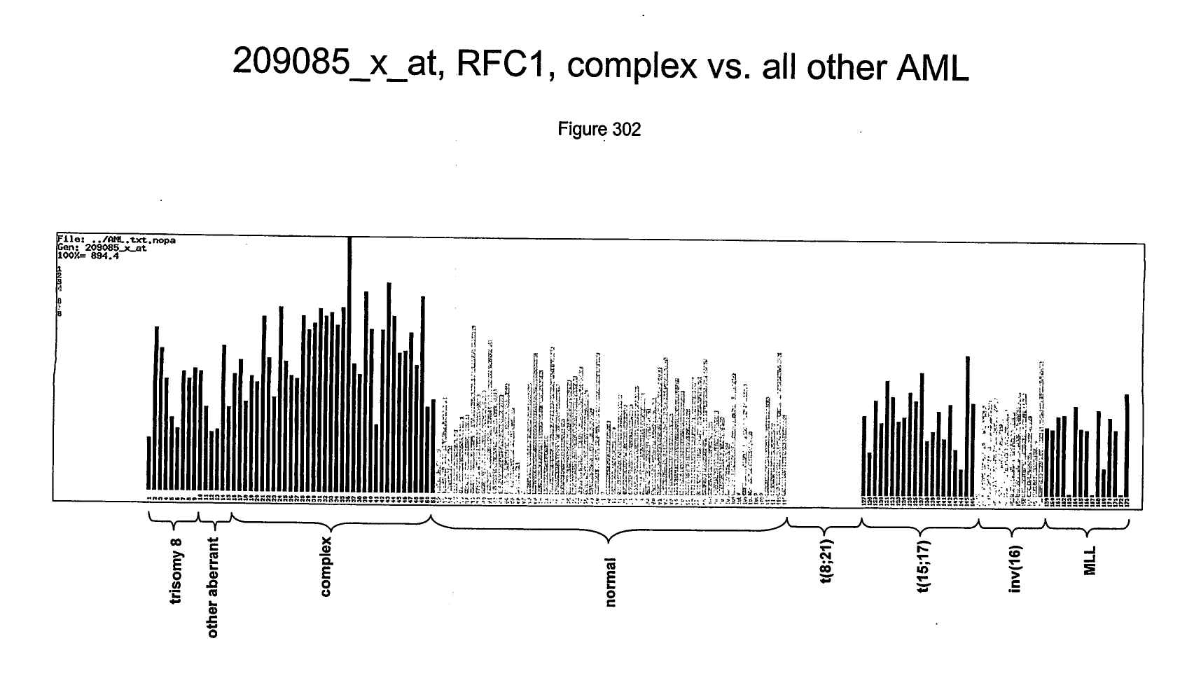

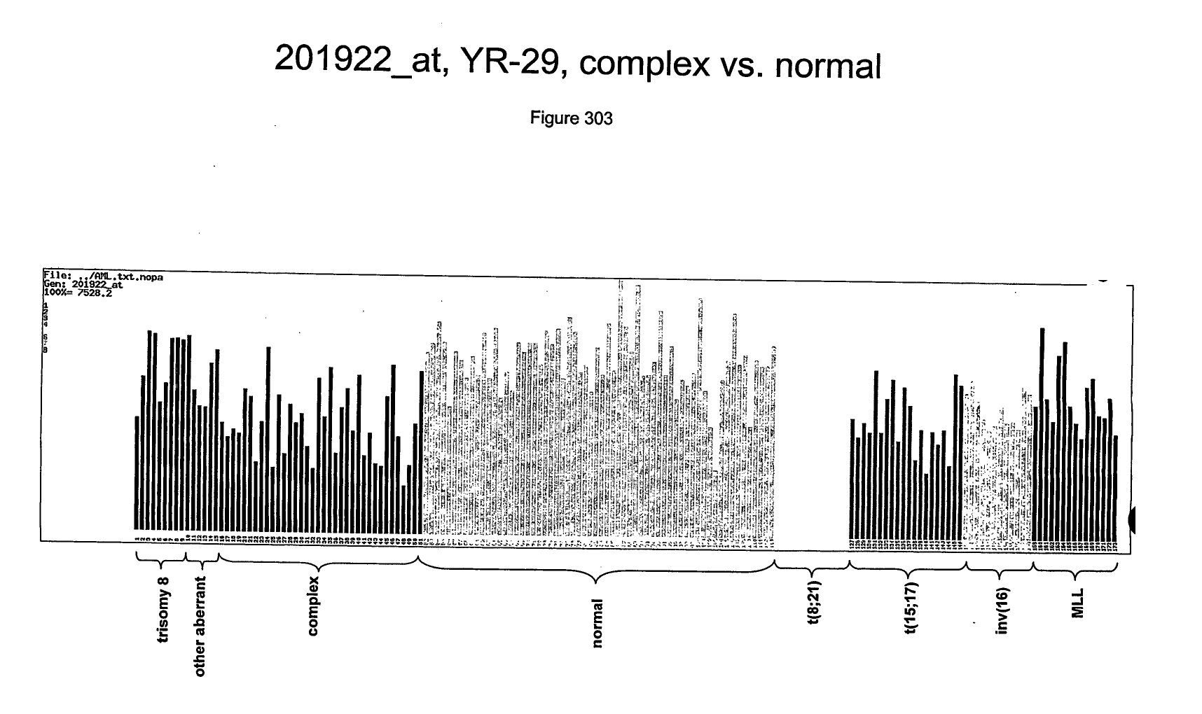

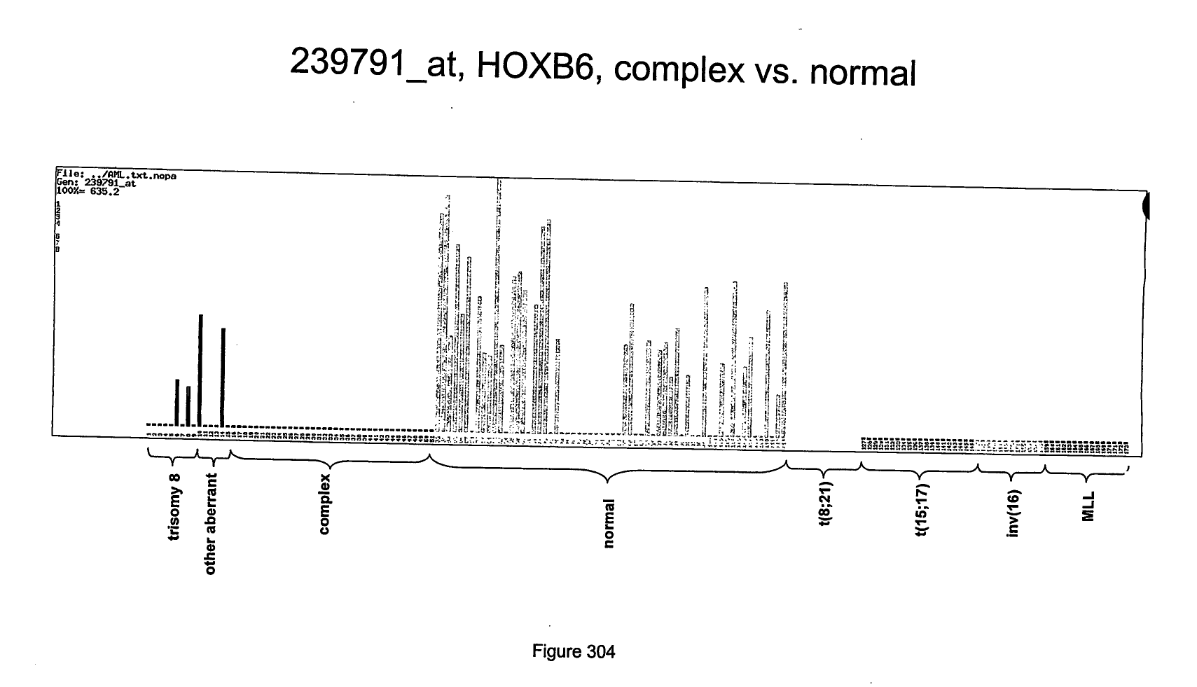

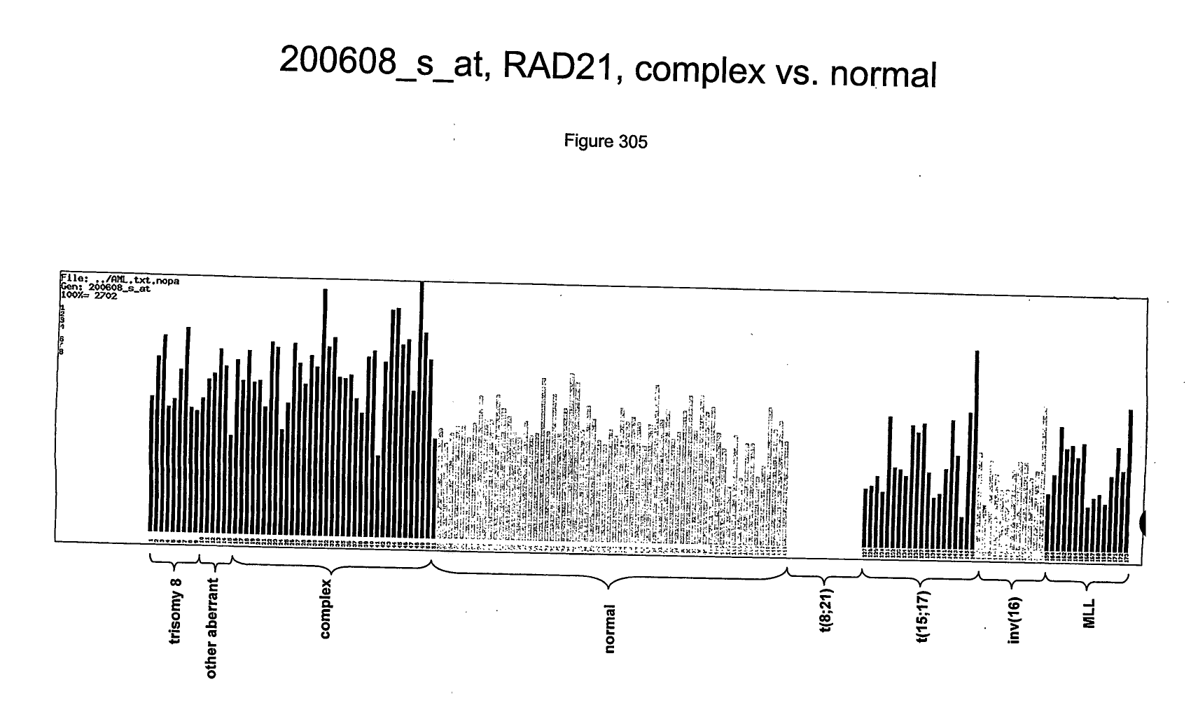

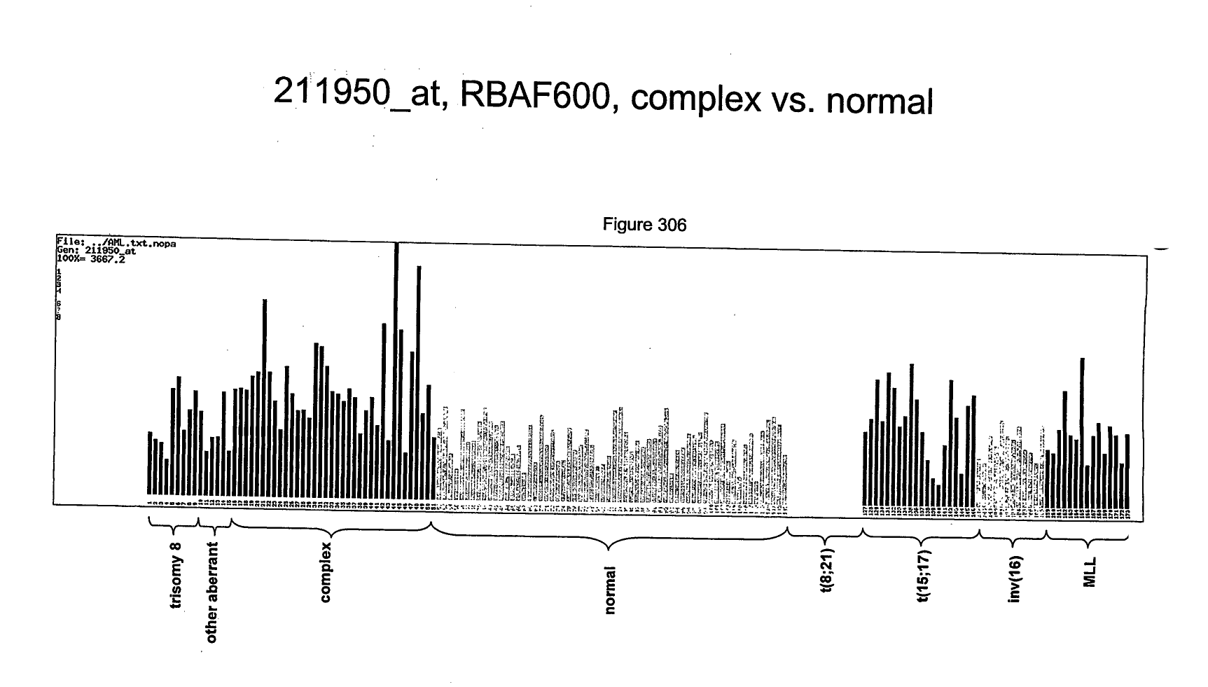

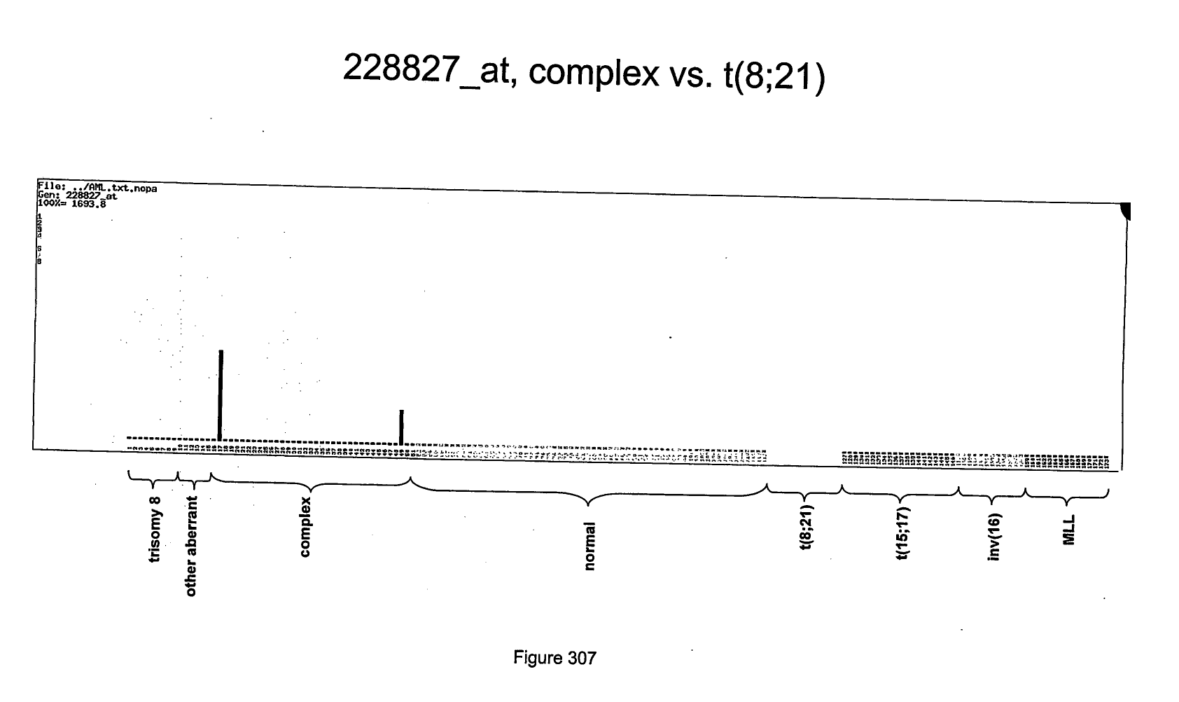

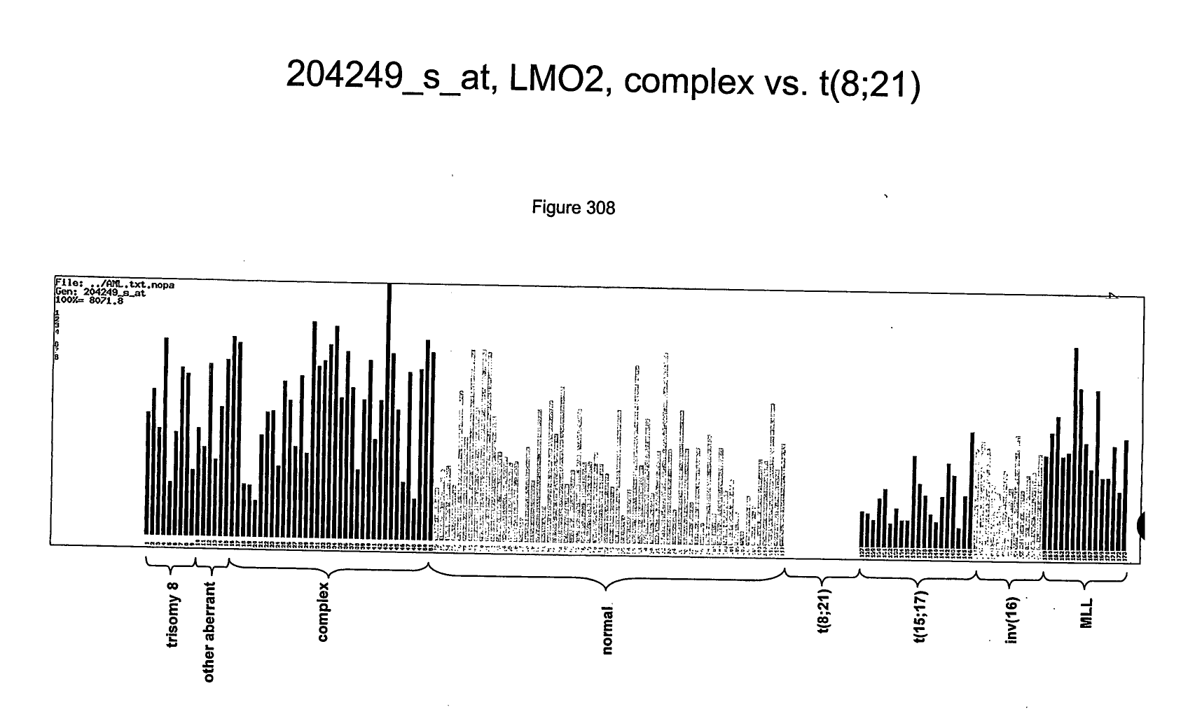

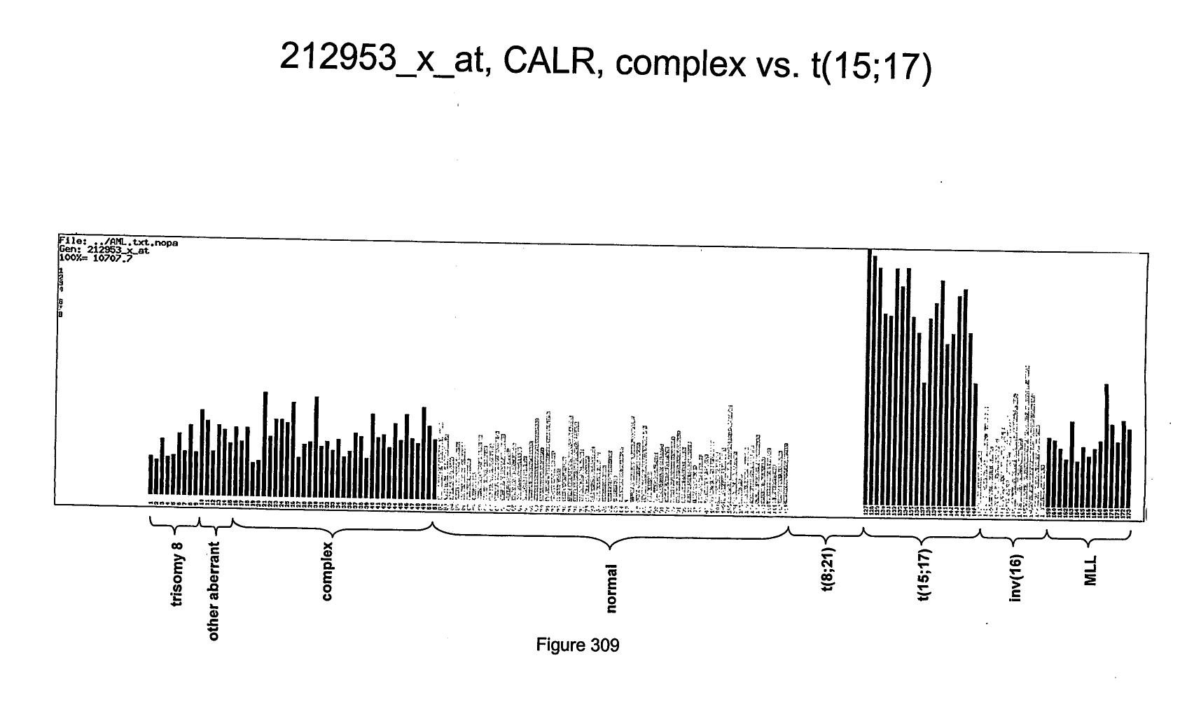

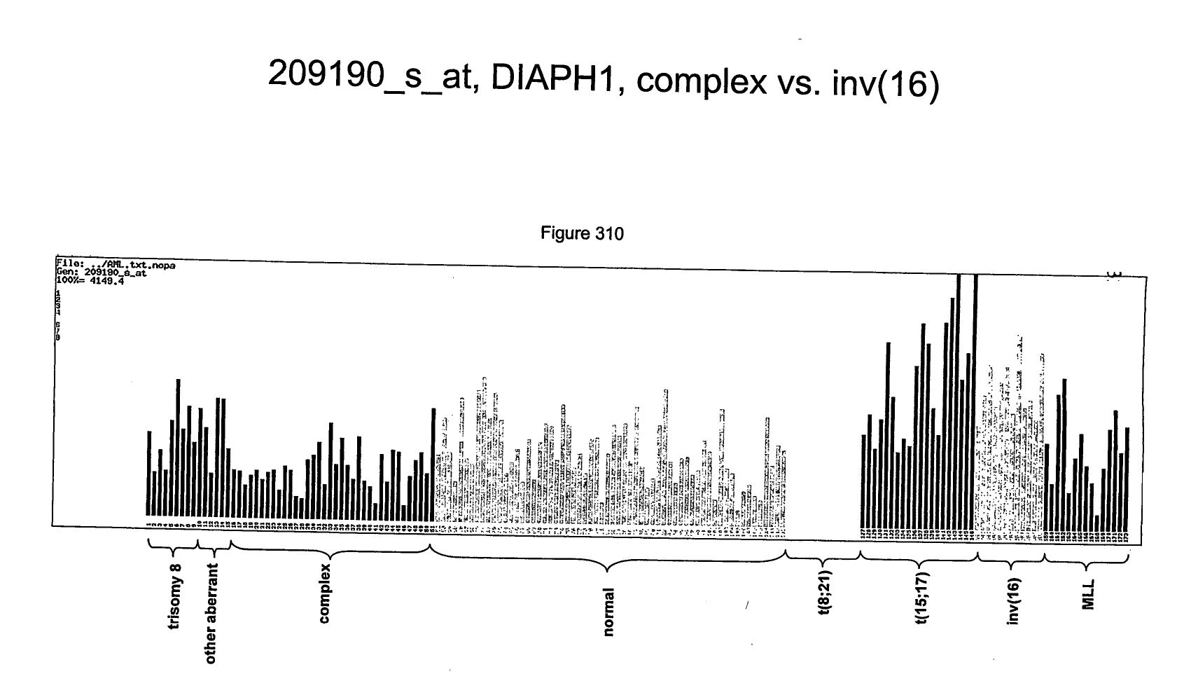

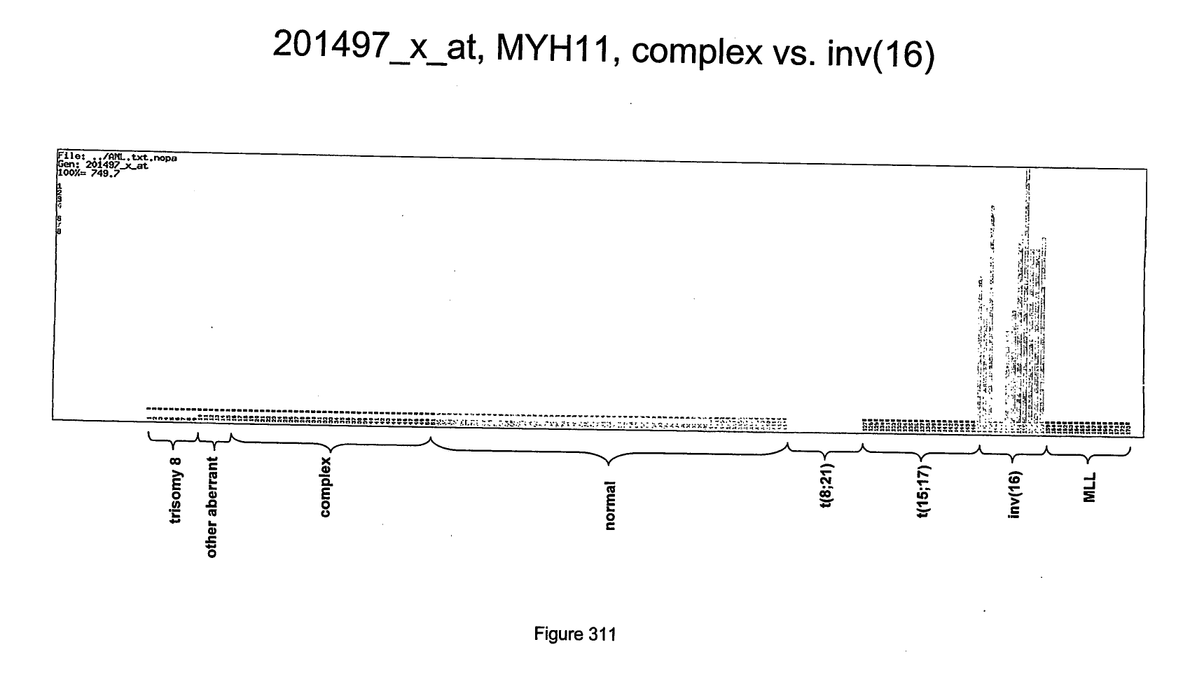

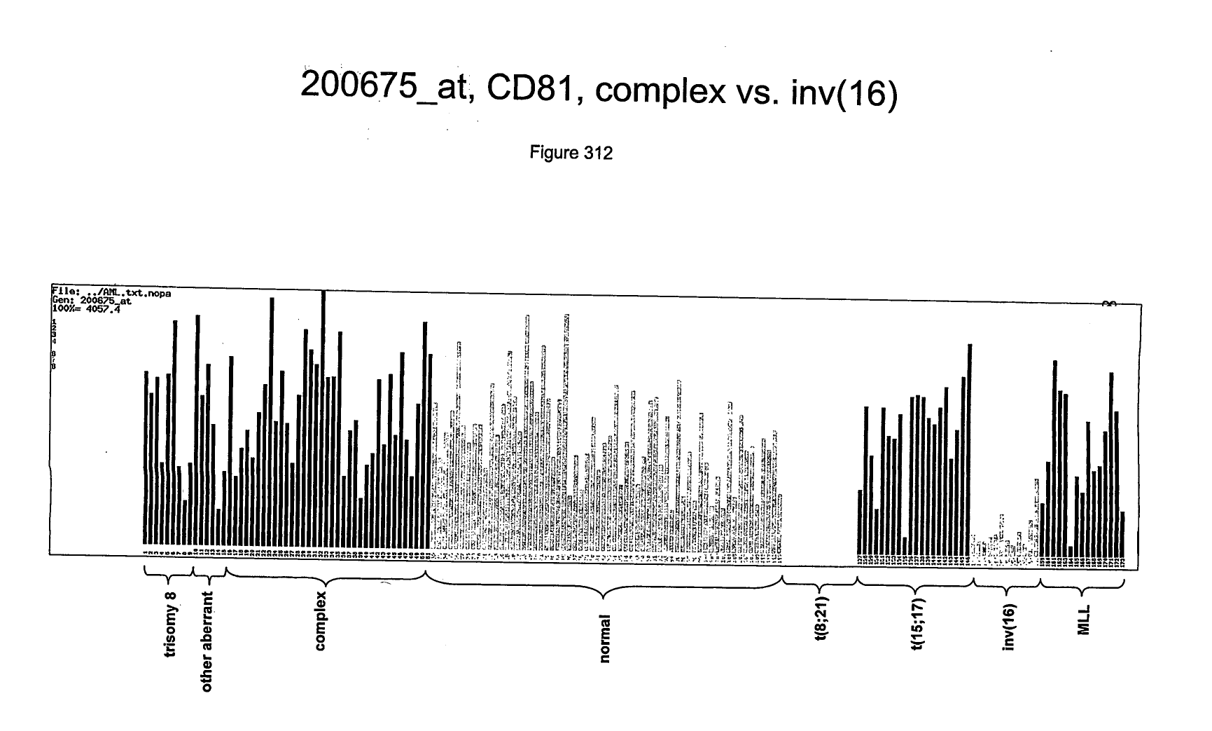

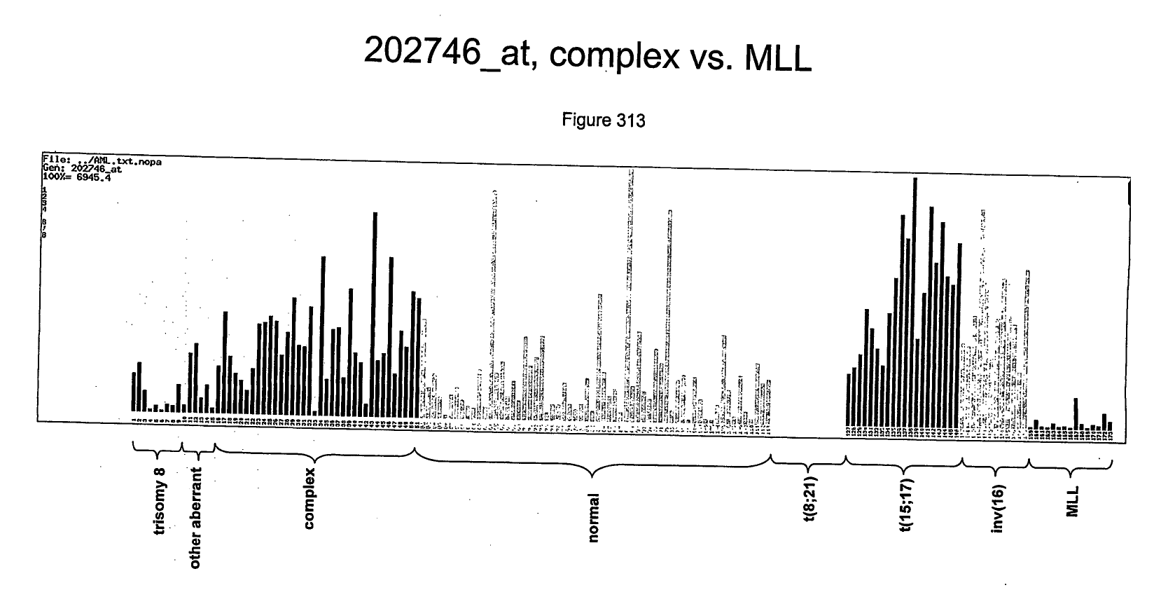

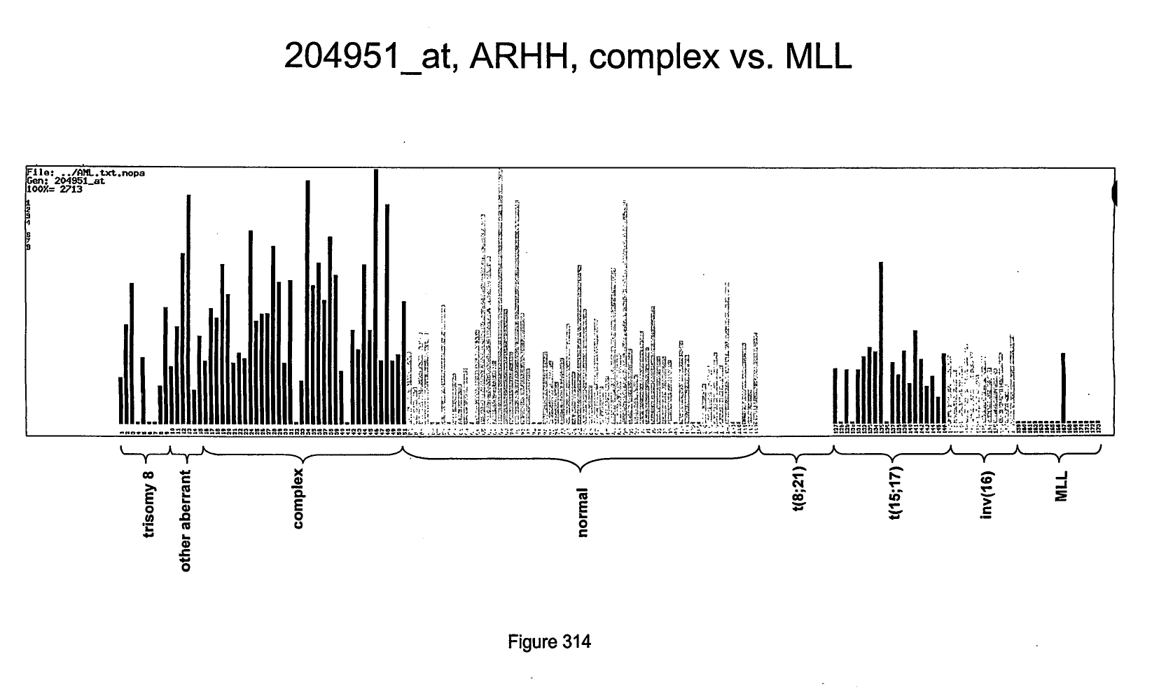

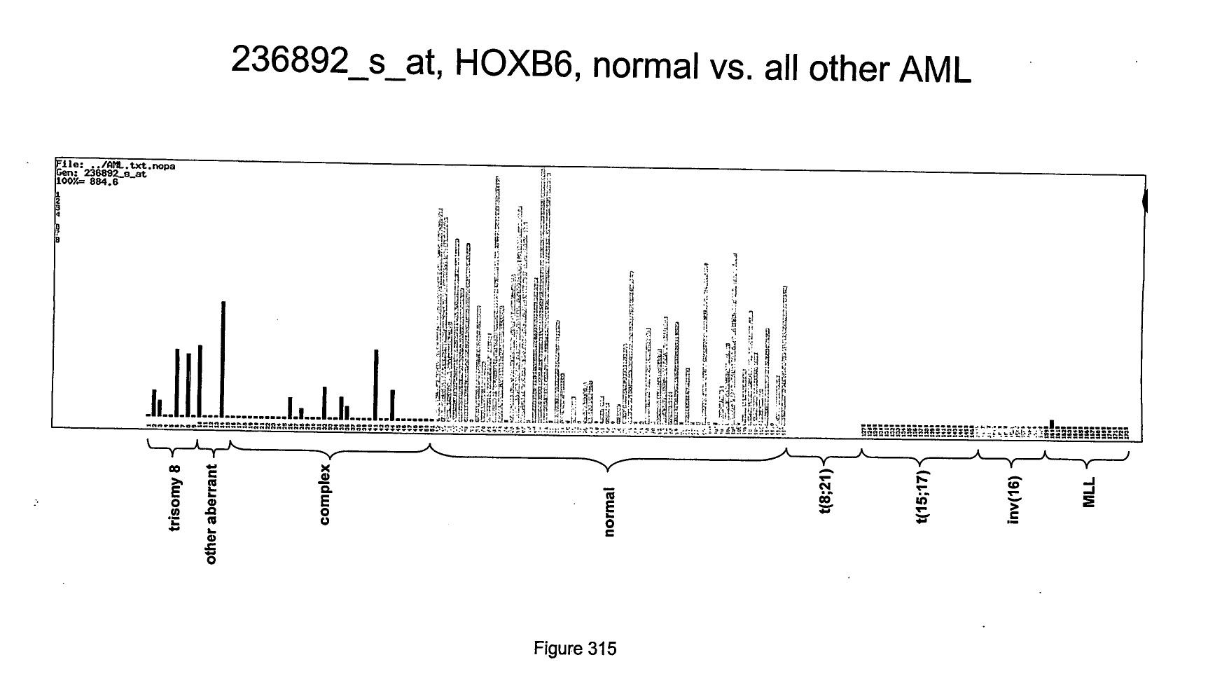

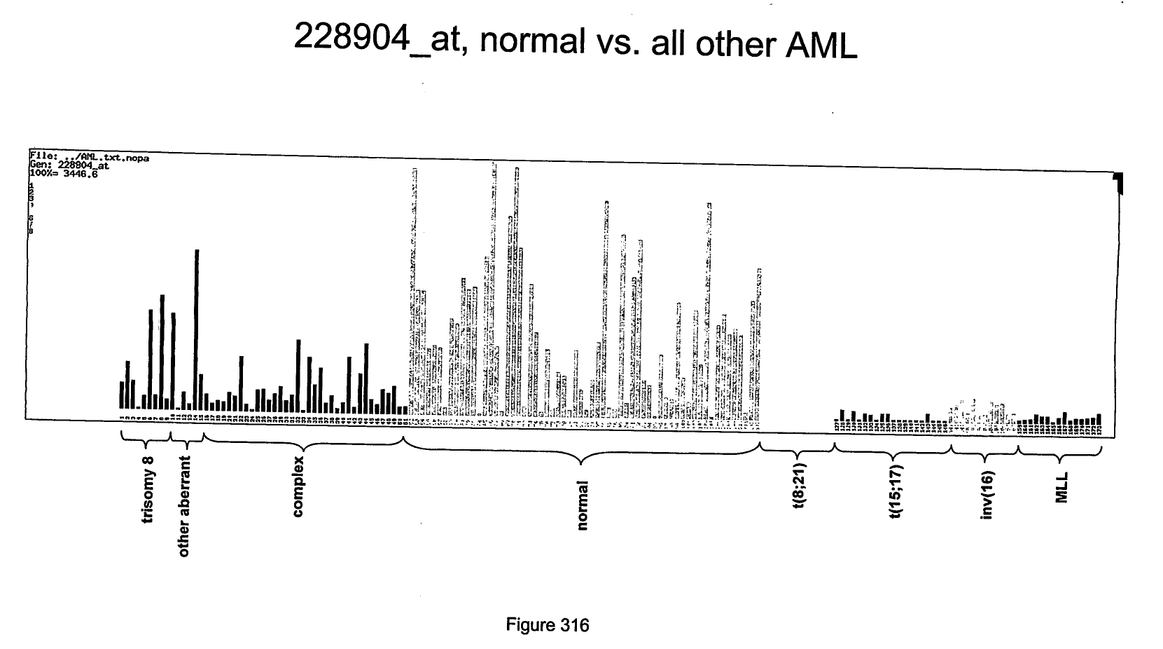

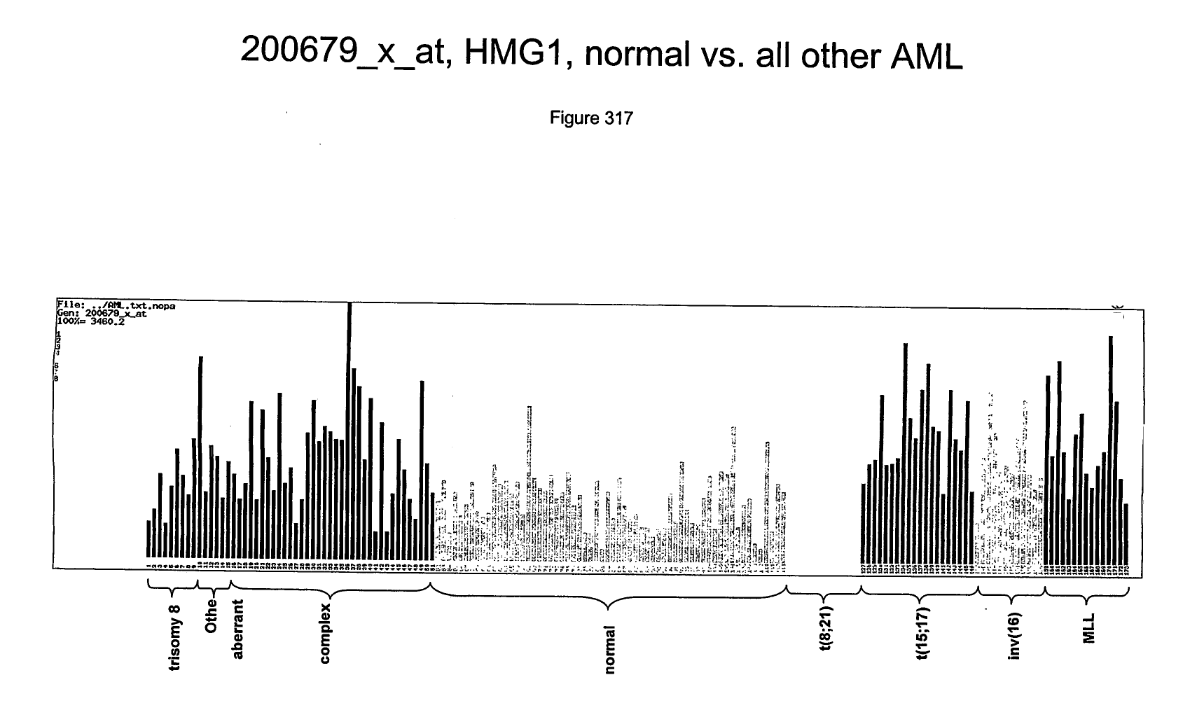

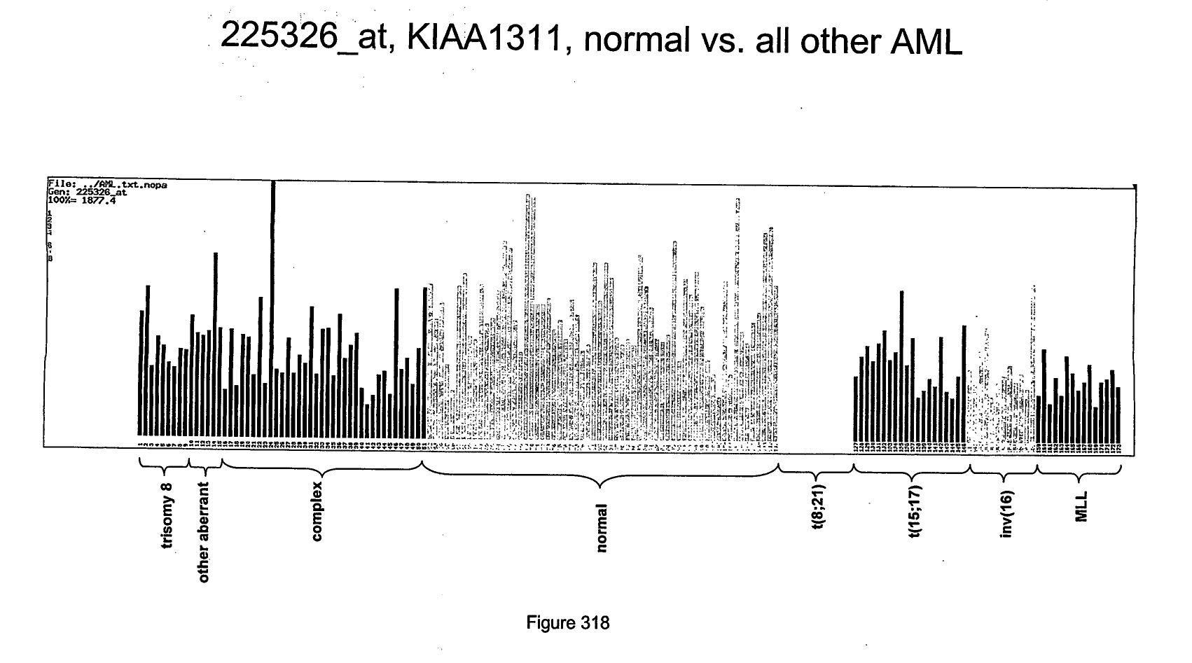

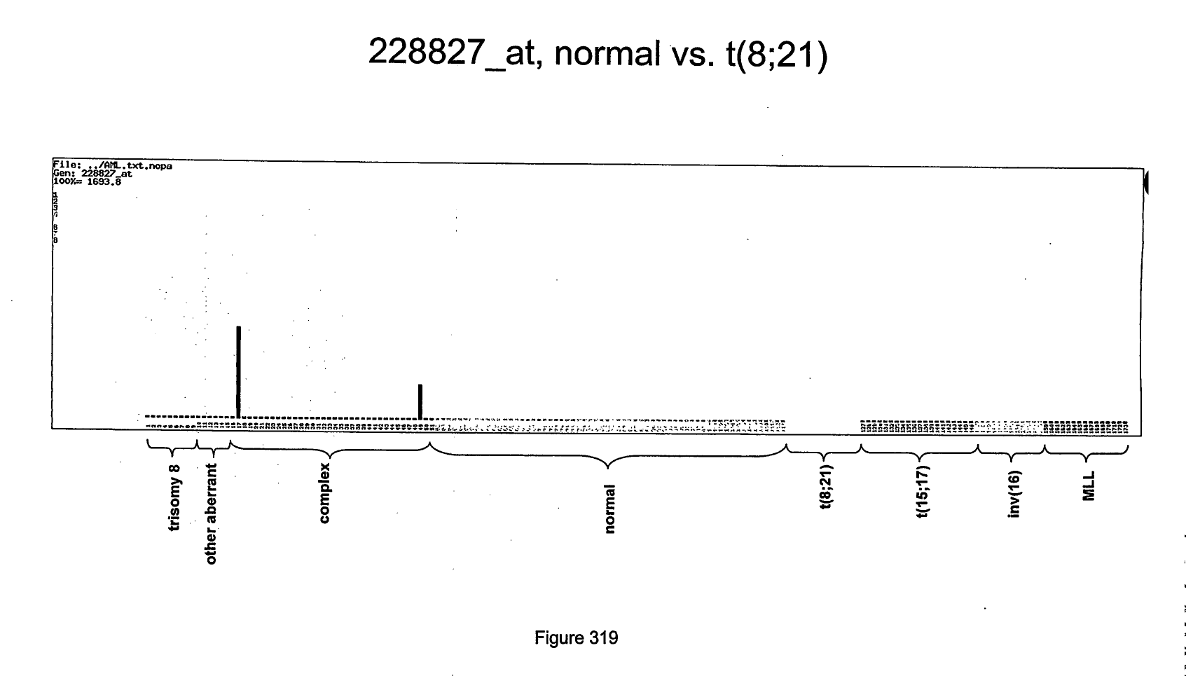

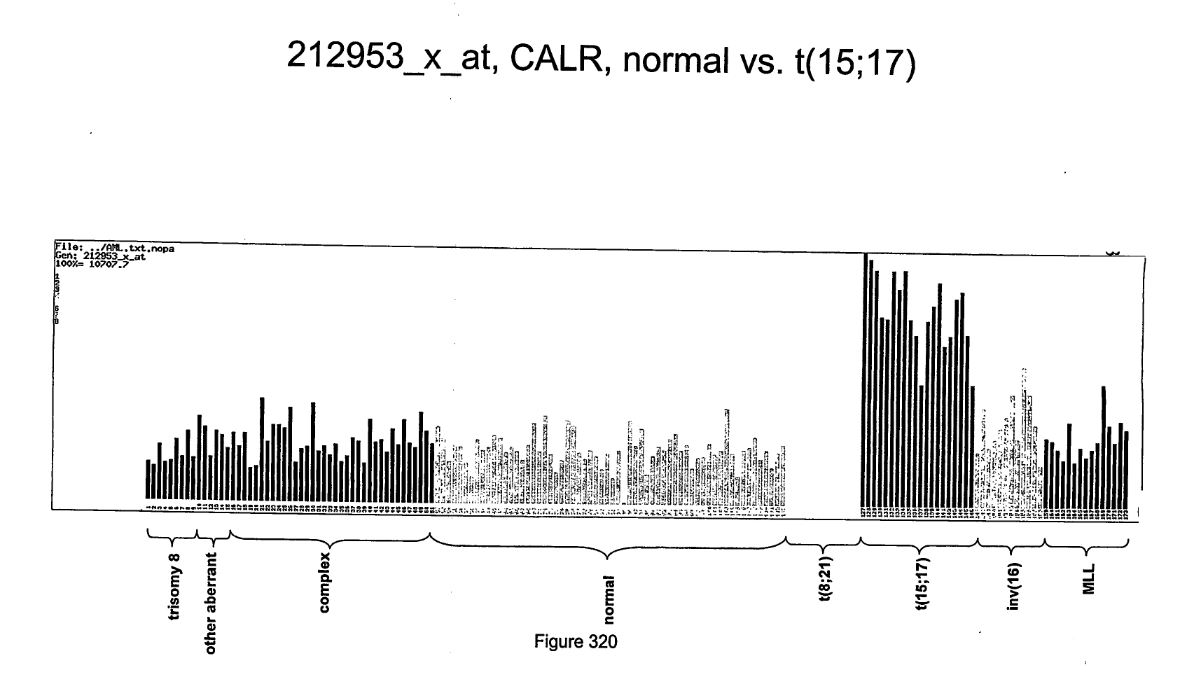

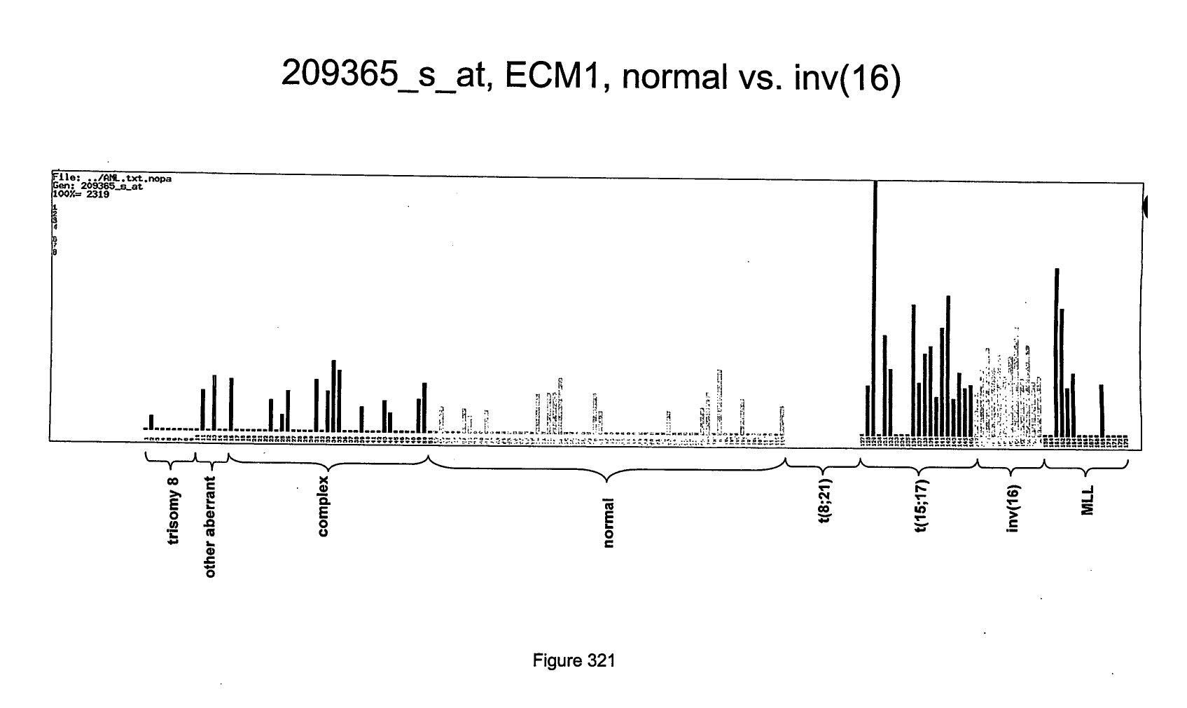

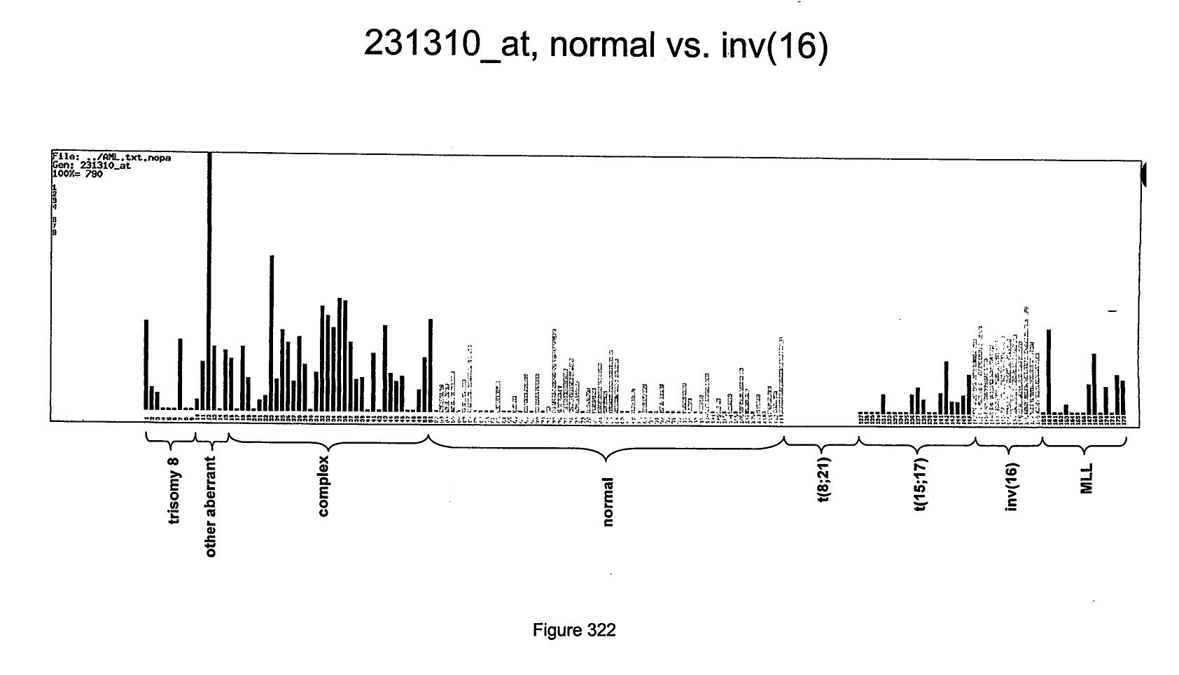

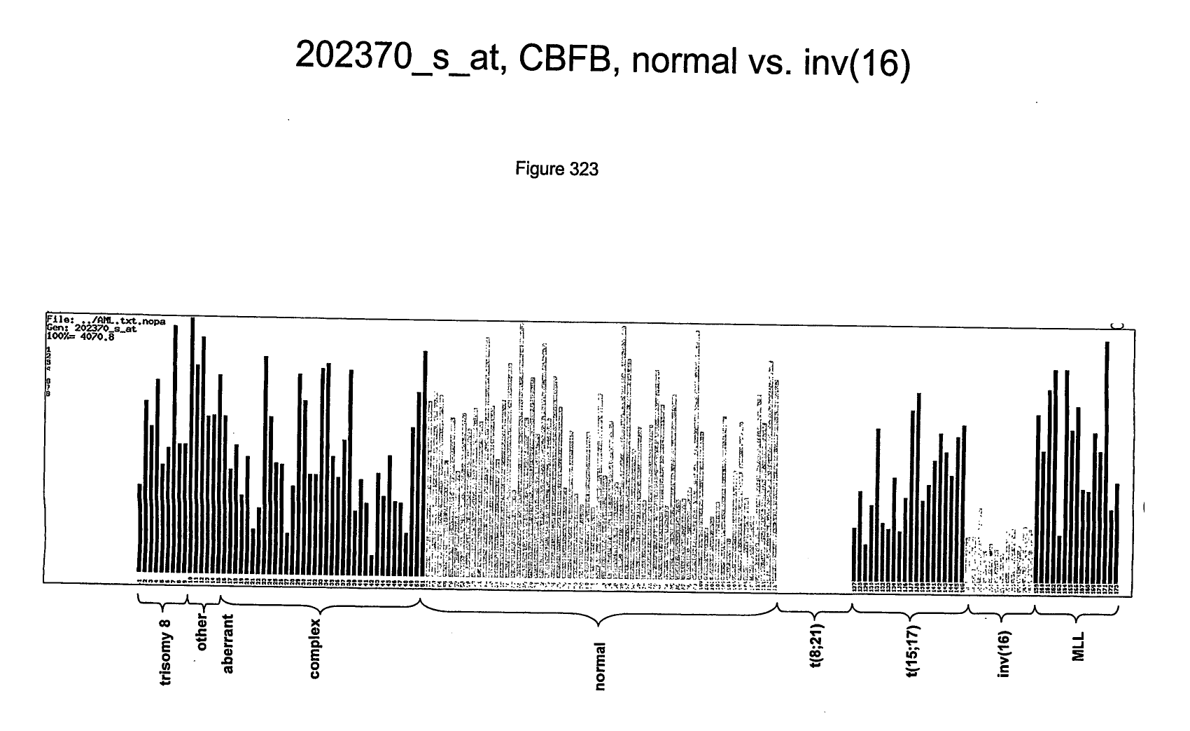

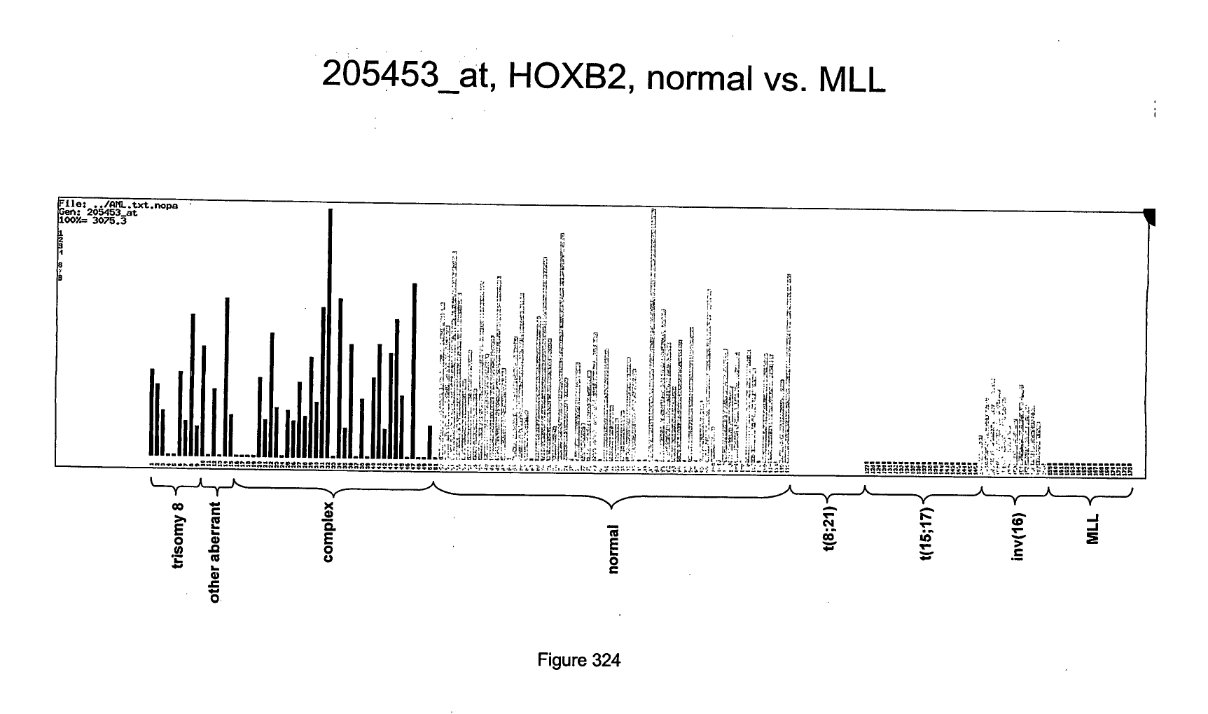

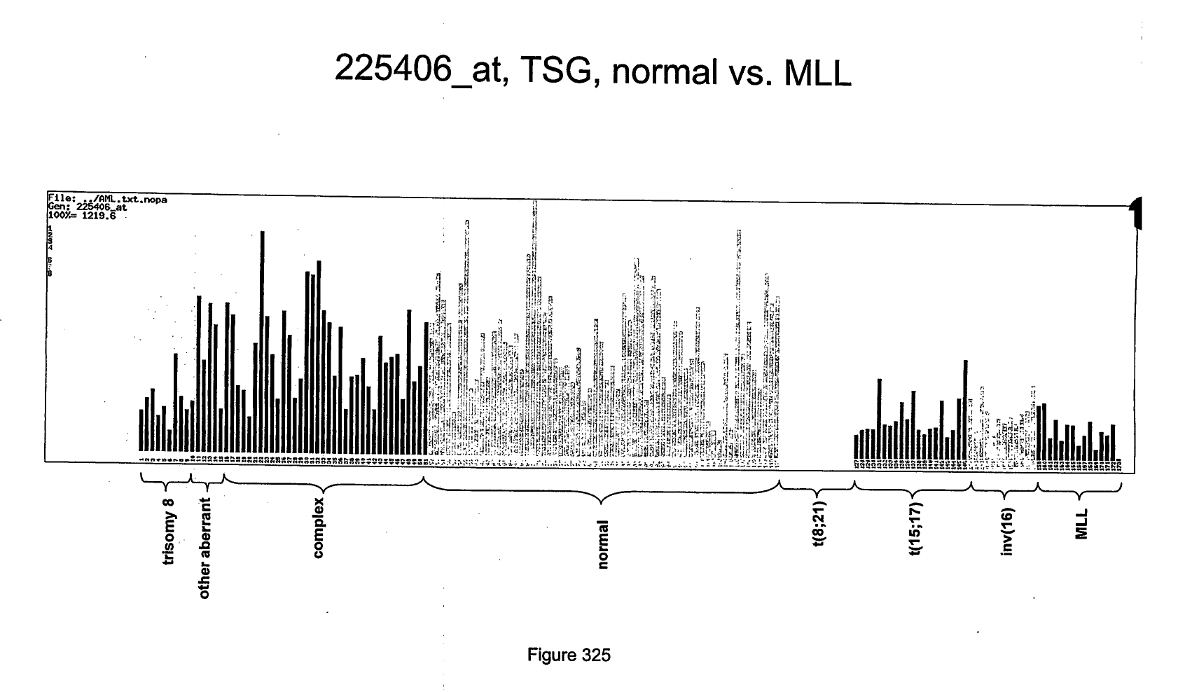

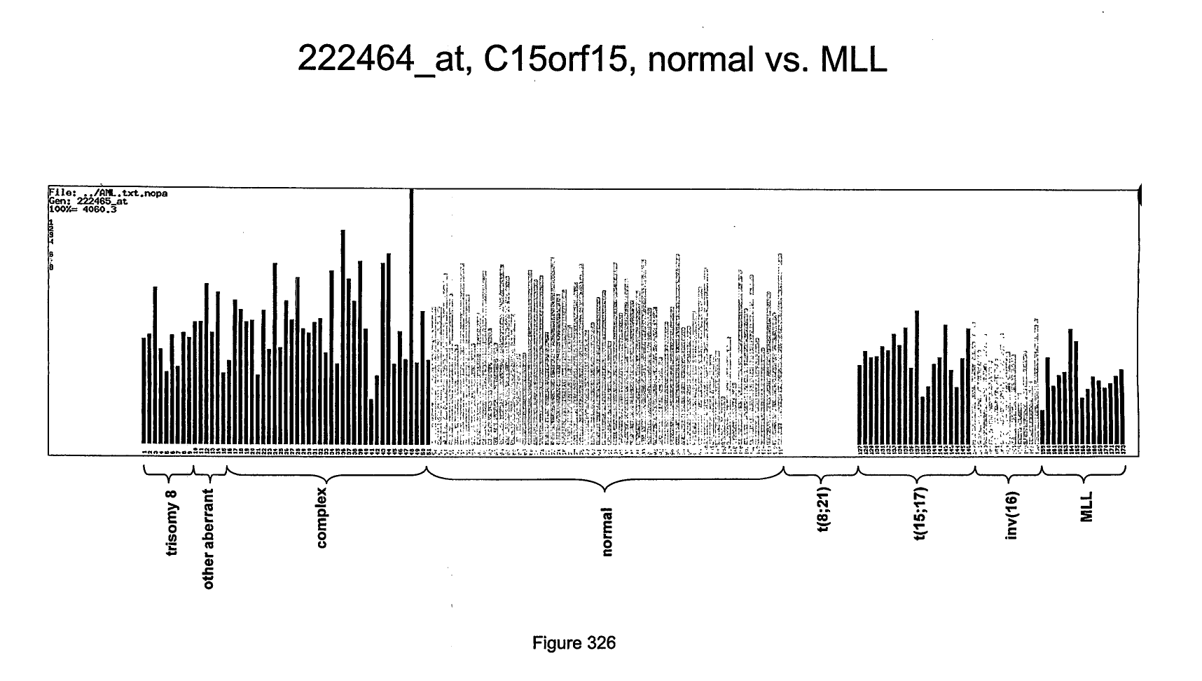

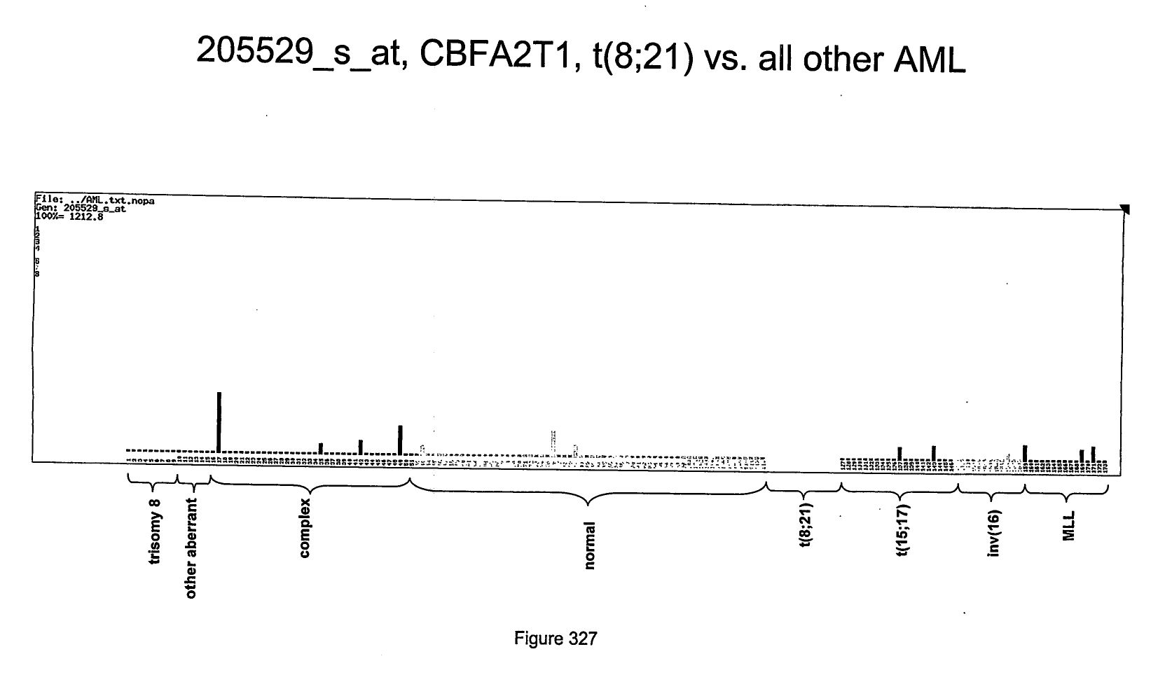

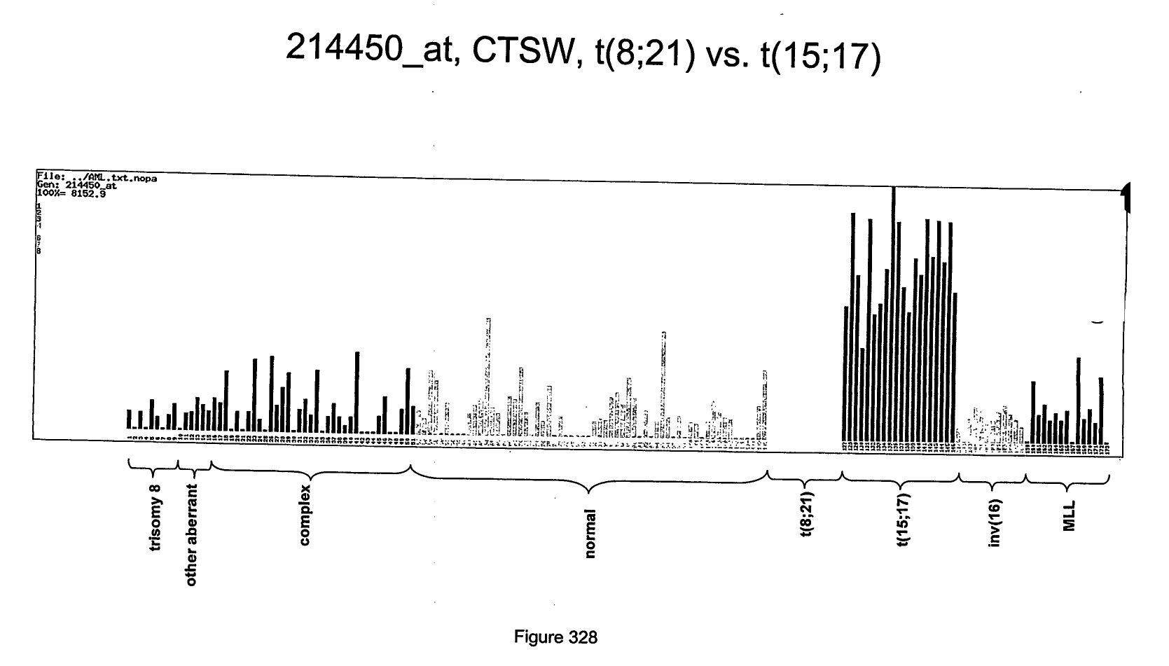

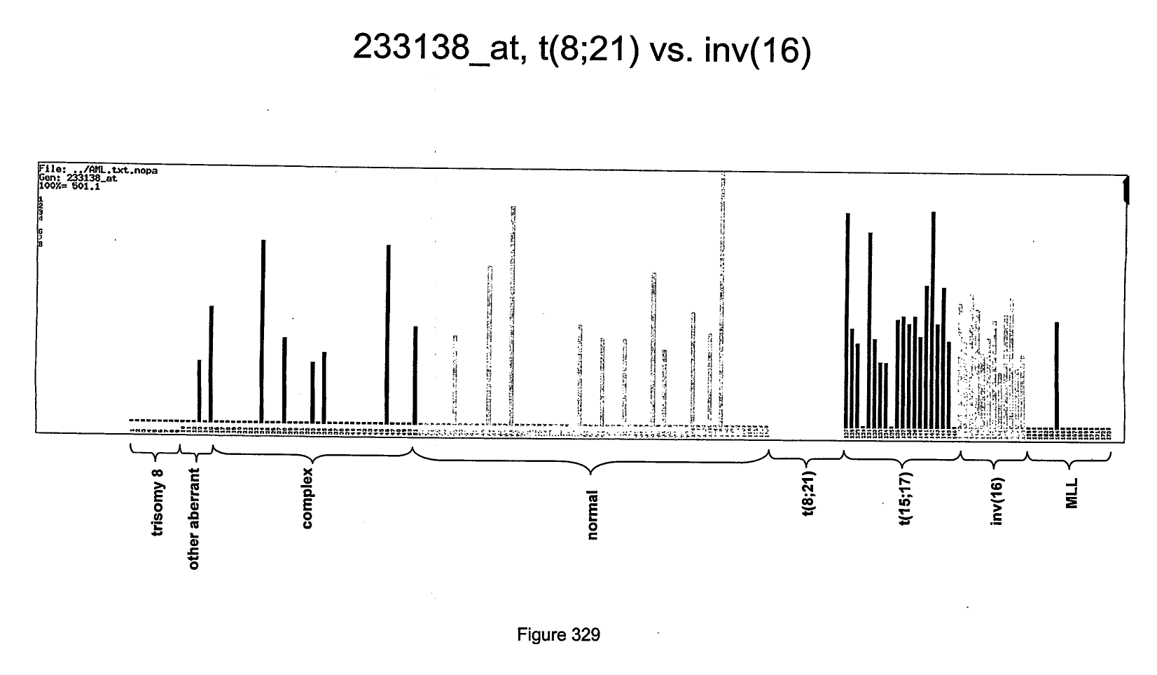

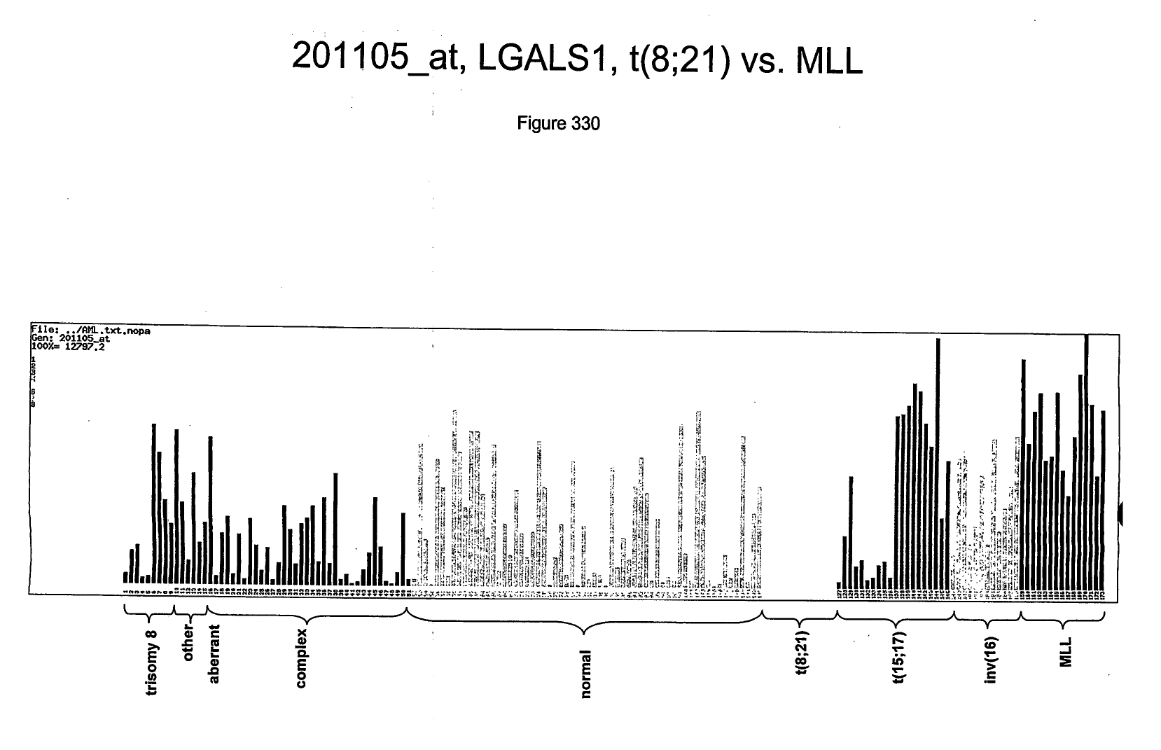

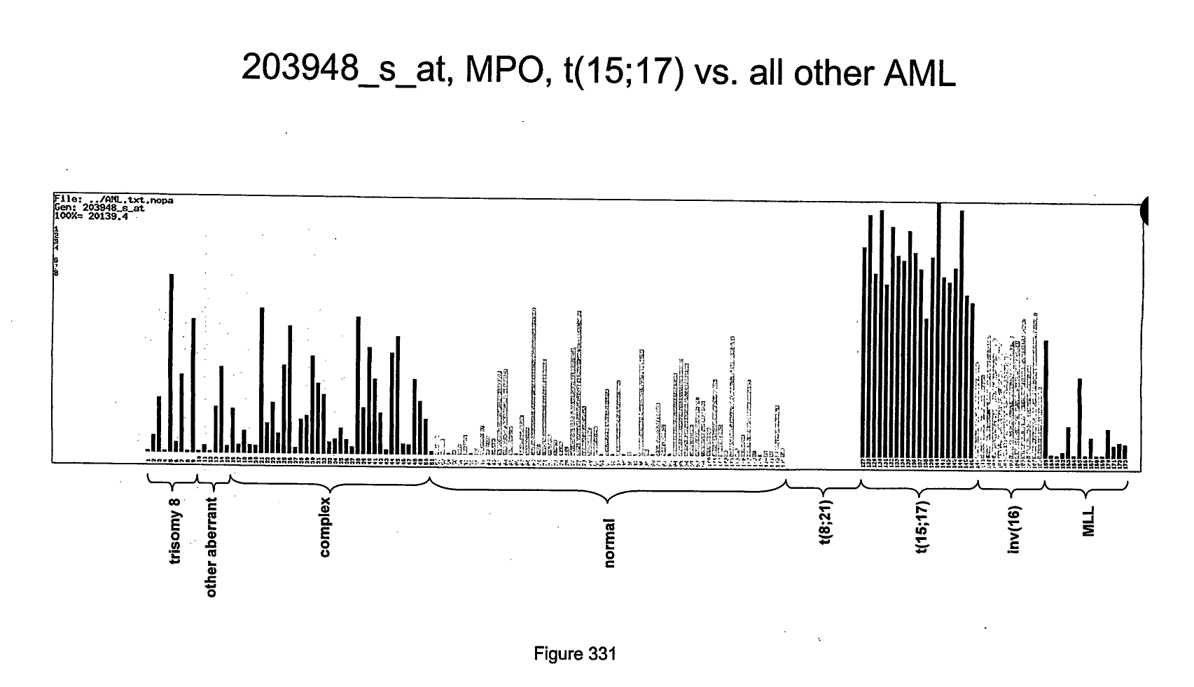

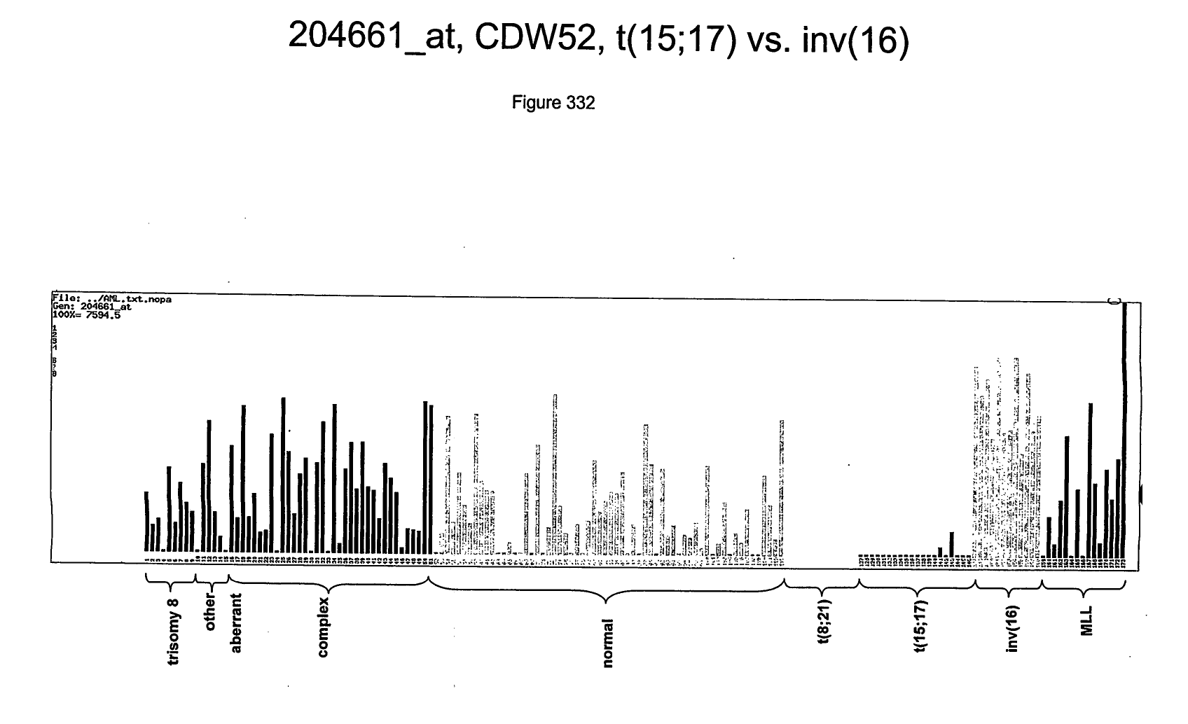

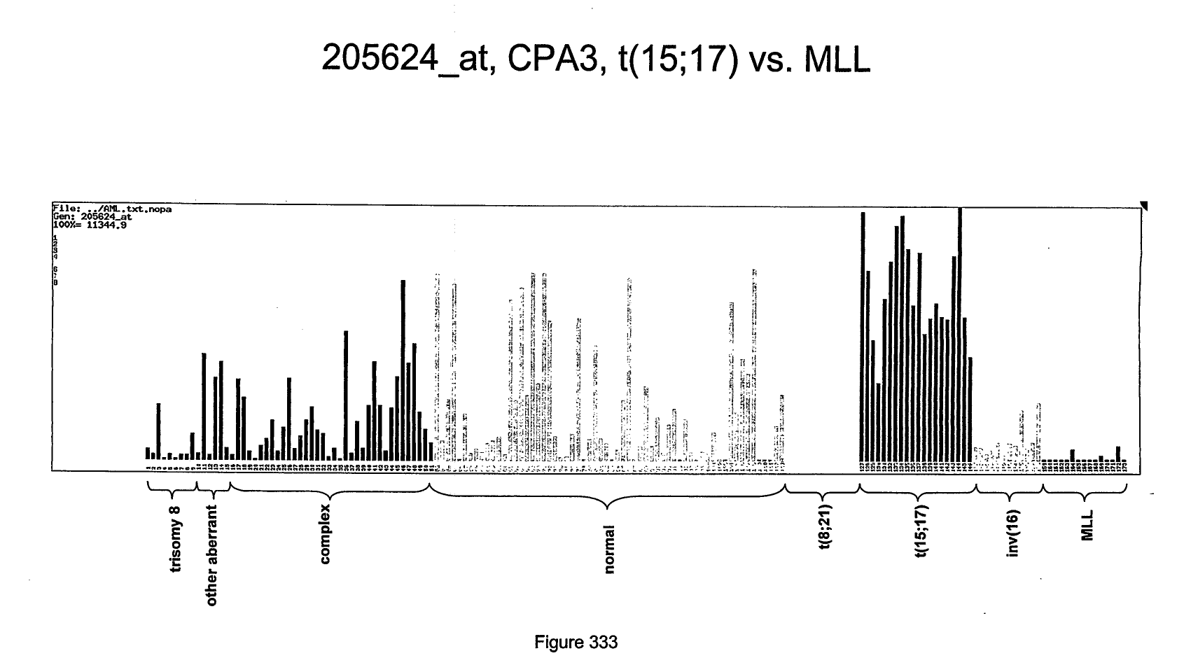

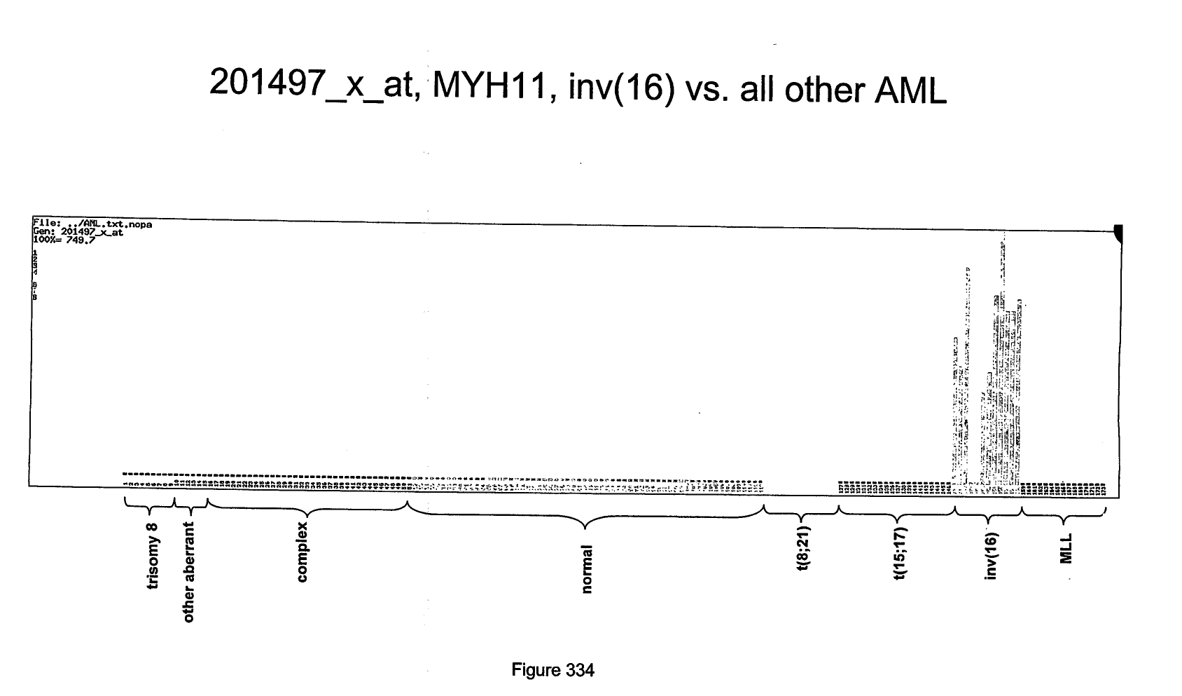

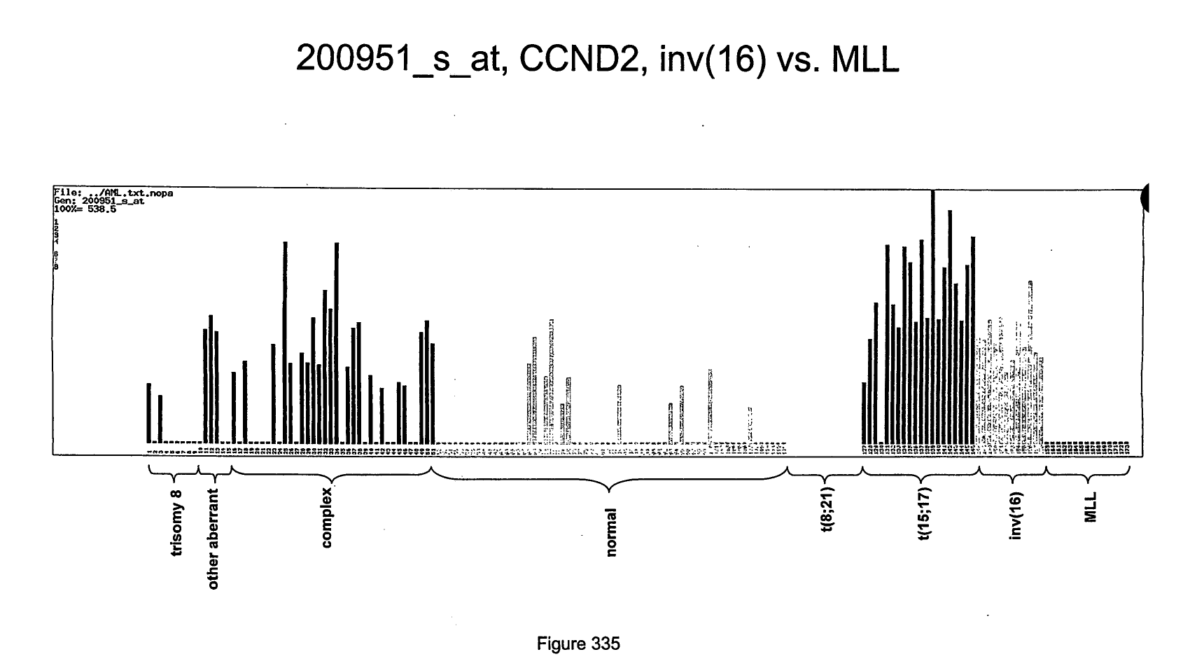

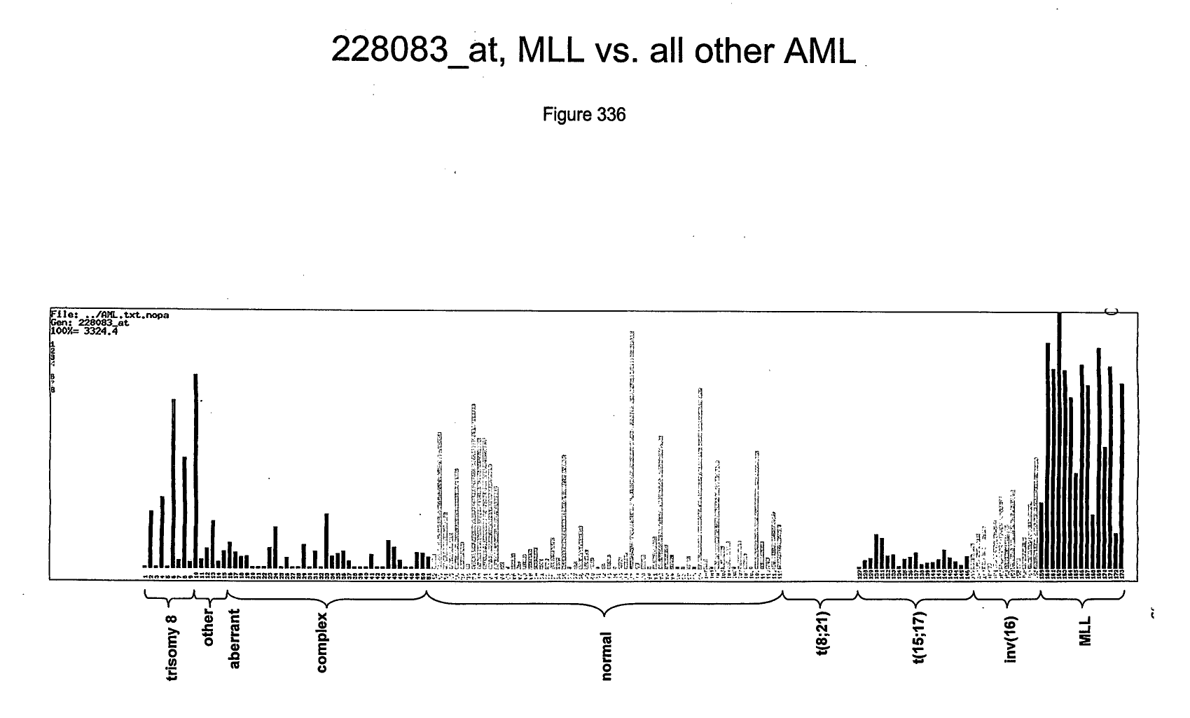

A set of 20 top-ranked genes, which provided both optimal classification accuracy and highest prediction strength for all pairwise (all pairs) and one-versus-all comparisons is given as table 35. Within this set of genes, optimal classification accuracy can be obtained with genes marked by asterisks. Gene expression intensities, plotted as bar graphs are given in FIGS. 272 to 336. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

In total 173 cases of AML were analyzed. 160 of 174 cases (92.5%) were assigned to the correct AML subtype in all pairwise comparisons (table 34a). The sensitivity indicated for each subgroup indicates the percentage of cases of the specific subgroup identified correctly in all pairwise comparisons (range 60% to 100%). The specificity indicates for each subgroup the percentage of correct assignments to this subgroup (range 85.5% to 100%).

In total 1211 individual assignments of AML were analyzed. 1198 of 1211 assignments (98.9%) were correct (table 34b). The sensitivity indicated for each subgroup indicates the percentage of correct assignments for cases of the specific subgroup in pairwise comparisons (range 94.3% to 100%). The specificity indicates for each subgroup the percentage of correct assignments to this subgroup (range 97.7% to 100%).

In a second approach significant genes were identified according to Westfall & Young. Table 36 represents all genes found to be significant after p-value adjustment. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

Furthermore, we provide information about genes which were found to be rated significant independently by both methodologies (Table 36). Top-significant genes according to the method of example 3 are marked by asterisks. Genes which were included in any of the top-20 lists are marked by positive signs.

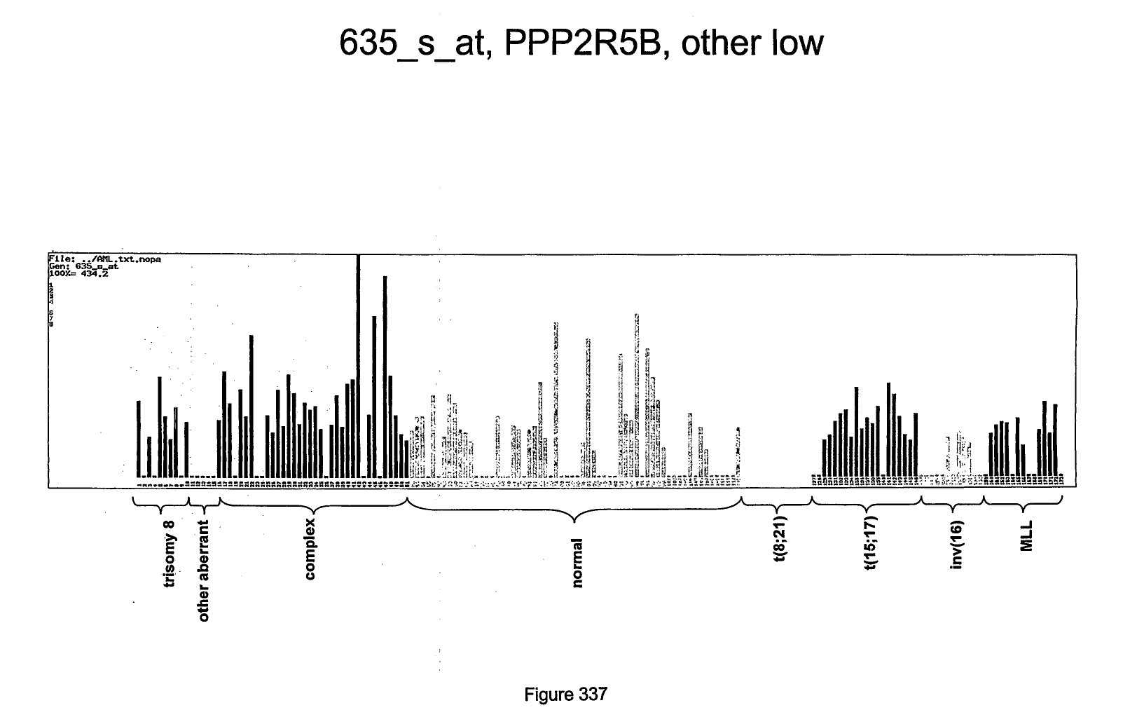

In addition, selected gene profiles were chosen to demonstrate their capability of discriminating different leukemia types, subtypes and normal bone marrow, respectively. Gene expression profiles were generated by means of PERL-programs, evaluated and plotted as bar graphs. Each of the analyzed groups are accordingly outlined. The following genes were selected and are given as FIGS. 337 to 370:

| GeneID | gene symbol | feature |
|---|---|---|
| 201497_x_at | MYH11 | AML inv(16) high |
| 228827_at | Homo sapiens clone 25023 mRNA | AML t(8; 21) high |
| | sequence | |
| 38487_at | FLJ12442 | AML t(15; 17) high |
| 203074_at | ANXA8 | AML t(15; 17) high |
| 205528_s_at | CBFA2T1 | AML t(8; 21) high |
| 205529_s_at | CBFA2T1 | AML t(8; 21) high |
| 206940_s_at | POU4F1 | AML t(8; 21) high |
| 211341_at | POU4F1 | AML t(8; 21) high |
| 201496_x_at | MYH11 | AML inv(16) high |
| 228660_x_at | SEMA4F | other high |
| 202718_at | IGFBP2 | AML t(15; 17) high |
| 205380_at | PDZK1 | other high |
| 202746_at | | AML MLL low |
| 201596_x_at | KRT18 | AML t(8; 21) low |
| 34210_at | CDW52 | AML t(15; 17) low |
| 212850_s_at | LRP4 | AML inv(16) high |
| 228904_at | ESTs | AML t(8; 21) low, |
| | | AML t(15; 17) low, |
| | | AML inv(16) low, |
| | | AML MLL low |
| 203151_at | MAP1A | AML t(8; 21) low |
| 201137_s_at | HLA-DPB1 | AML t(15; 17) low |
| 200675_at | CD81 | AML inv(16) low |
| 201425_at | ALDH2 | AML t(8; 21) low |
| 202085_at | TJP2 | AML inv(16) low |
| 202619_s_at | PLOD2 | AML MLL low |
| 203092_at | TIMM44 | AML inv(16) low |
| 204425_at | ARHGAP4 | AML t(15; 17) low |
| 205366_s_at | HOXB6 | AML t(8; 21) low, |
| | | AML t(15; 17) low, |
| | | AML inv(16) low, |
| | | AML MLL low |
| 205472_s_at | DACH | AML MLL high |
| 206761_at | TACTILE | AML MLL low |
| 222166_at | | AML +8 low |
| 222335_at | ESTs | AML MLL low |
| 223318_s_at | MGC10974 | AML complex low |
| 225330_at | Homo sapiens, | AML inv(16) low |
| | clone MGC: 18216 | |
| | IMAGE: 4156235, | |
| | mRNA, complete cds | |
| 231277_x_at | ESTs | AML complex low |
| 635_s_at | PPP2R5B | other low |

### Example 7

### Results VII: Analysis of 5 Genetically Defined CLL Subtypes

Here we analyzed in total 5 genetically defined CLL subtypes. We applied the described two different statistical methods for identification of genes which allow accurate class assignments to the respective groups.

| | |
|---|---|
| trisomy 12 | (n = 5) |
| 11q- | (n = 4) |
| 13q- | (n = 10) |
| 17p- | (n = 4) |
| normal | (n = 9) |

First, expression data were analyzed according to example 3 as described hereinabove.

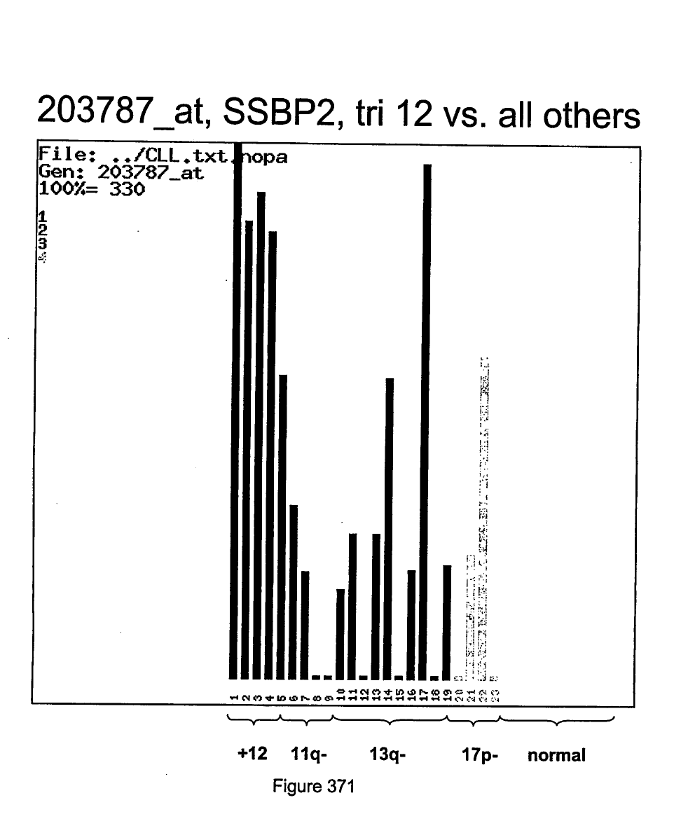

A set of 20 top-ranked genes, which provided both optimal classification accuracy and highest prediction strength for all pairwise (all pairs) and one-versus-all comparisons is given as table 38. Within this set of genes, optimal classification accuracy can be obtained with genes marked by asterisks. Gene expression intensities, plotted as bar graphs are given in FIGS. 371 to 404. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

In total 32 cases of CLL were analyzed. 31 of 32 cases (96.9%) were assigned to the correct CLL subtype in all pairwise comparisons (table 37a). The sensitivity indicated for each subgroup indicates the percentage of cases of the specific subgroup identified correctly in all pairwise comparisons (range 90% to 100%). The specificity indicates for each subgroup the percentage of correct assignments to this subgroup (range 90% to 100%).

In total 128 individual assignments of CLL were analyzed. 127 of 128 assignments (99.2%) were correct (table 37b). The sensitivity indicated for each subgroup indicates the percentage of correct assignments for cases of the specific subgroup in pairwise comparisons (range 97.5% to 100%). The specificity indicates for each subgroup the percentage of correct assignments to this subgroup (range 97.3% to 100%).

In a second approach significant genes were identified according to Westfall & Young. Table 39 represents all genes found to be significant after p-value adjustment. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

Furthermore, we provide information about genes which were found to be rated significant independently by both methodologies (Table 39). Top-significant genes according to the method of example 3 are marked by asterisks. Genes which were included in any of the top-20 lists are marked by positive signs.

### Example 7

### Results VIII: Analysis of the Four Major Leukemia Types (ALL, AML CLL, CML) and Normal Bone Marrow

Here we analyzed in total 4 major leukemia types as well a cohort of healthy volunteers for normal bone marrow characteristics. We applied the described two different statistical methods for identification of genes which allow accurate class assignments to the respective groups.

ALL (n=47)

AML (n=175)

CLL (n=35)

CML (n=14)

Normal bone marrow (n=9)

First, expression data were analyzed according to example 3 as described hereinabove.

A set of 20 top-ranked genes, which provided both optimal classification accuracy and highest prediction strength for all pairwise (all pairs) and one-versus-all comparisons is given as table 41. Within this set of genes, optimal classification accuracy can be obtained with genes marked by asterisks. Gene expression intensities, plotted as bar graphs are given in FIGS. 405 to 431. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

In total 280 cases of leukemia and normal bone marrow (BM) were analyzed. 263 of 280 cases (93.9%) were assigned to the correct leukemia subtype or normal bone marrow in all pairwise comparisons (table 40a). The sensitivity indicated for each subgroup indicates the percentage of cases of the specific subgroup identified correctly in all pairwise comparisons (range 76.6% to 98.3%). The specificity indicates for each subgroup the percentage of correct assignments to this subgroup (range 88.9% to 97.1%).

In total 1120 individual assignments of leukemia subtype or normal bone marrow were analyzed. 1103 of 1120 assignments (98.5%) were correct (table 40b). The sensitivity indicated for each subgroup indicates the percentage of correct assignments for cases of the specific subgroup in pairwise comparisons (range 94.2% to 99.3%). The specificity indicates for each subgroup the percentage of correct assignments to this subgroup (range 97.2% to 99.3%).

In a second approach significant genes were identified according to Westfall & Young. Table 42 represents all genes found to be significant after p-value adjustment. Genes are depicted as unique Affymetrix identifier (for example 201497_x_at) and, where available, approved HGNC symbols (HUGO Gene Nomenclature Committee). More detailed, the complete annotation and sequence information about this set of genes is listed in table 43a,b.

Furthermore, we provide information about genes which were found to be rated significant independently by both methodologies (Table 42). Top-significant genes according to the method of example 3 are marked by asterisks. Genes which were included in any of the top-20 lists are marked by positive signs.

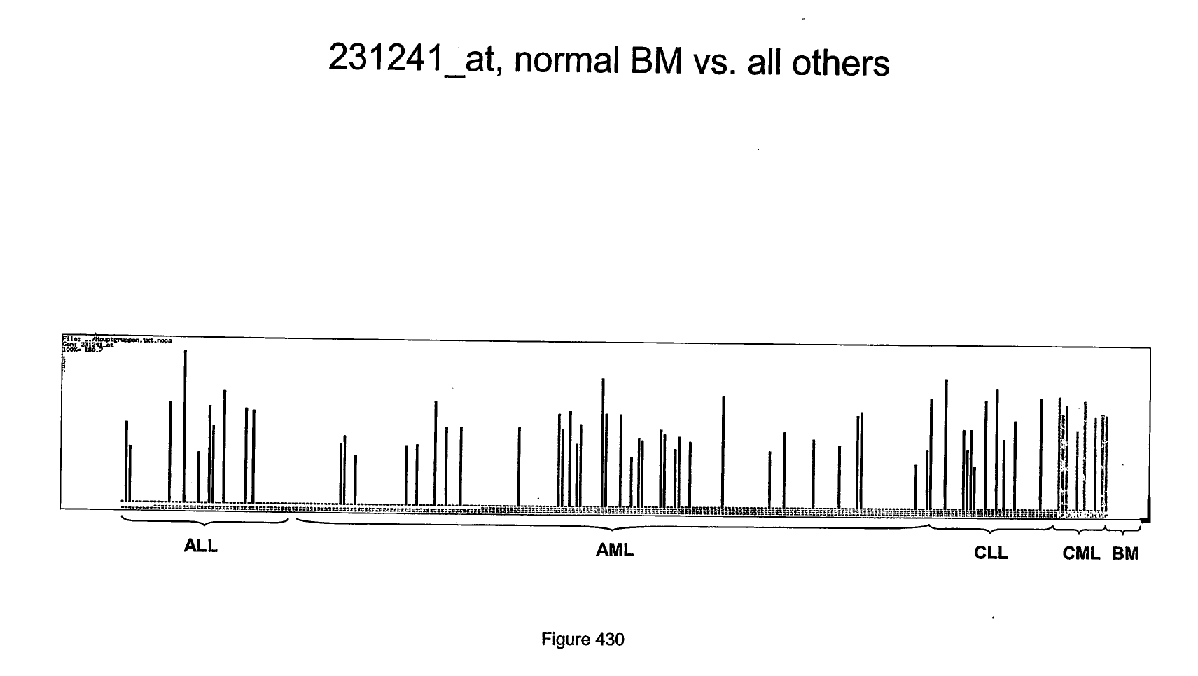

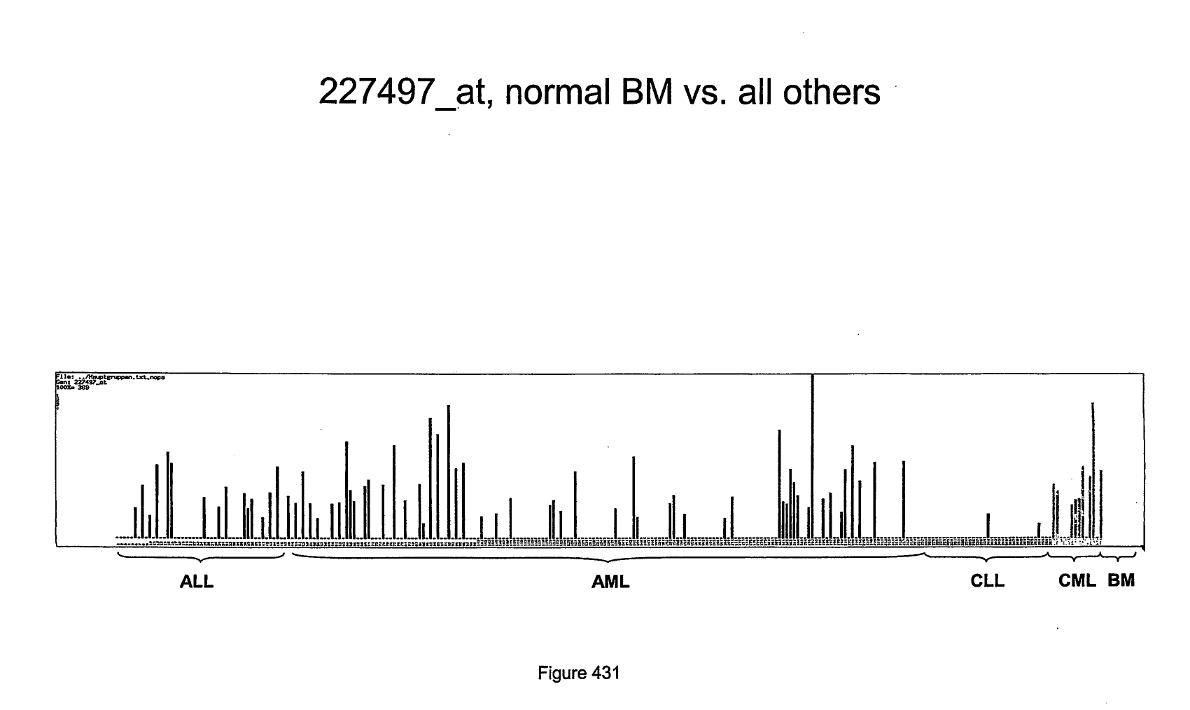

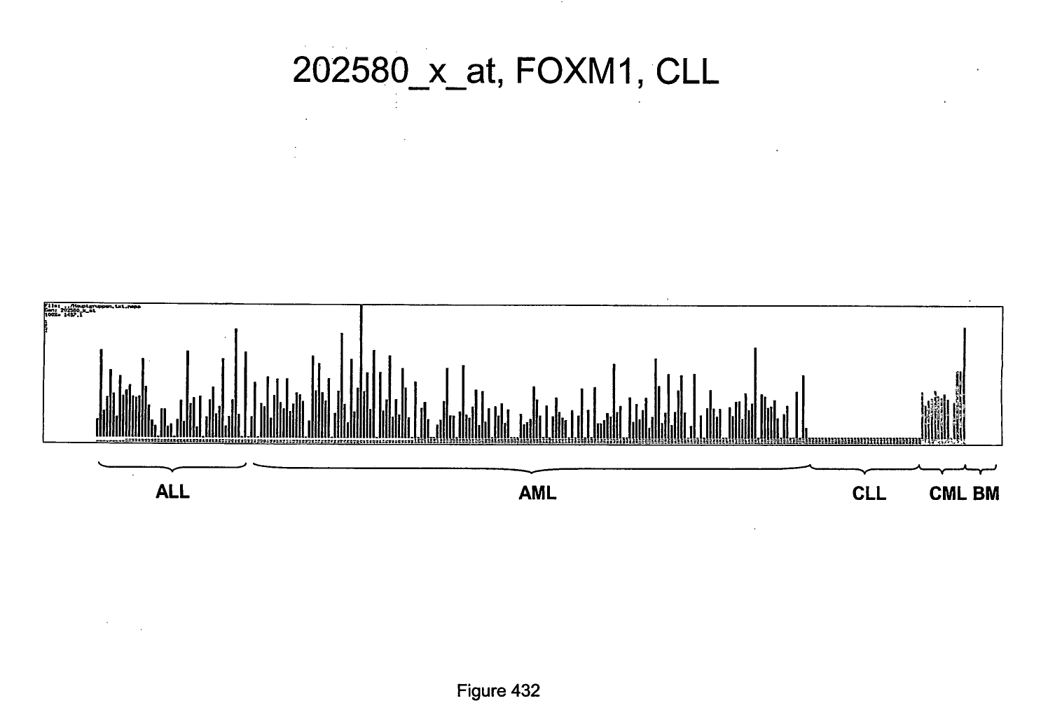

In addition, selected gene profiles were chosen to demonstrate their capability of discriminating different leukemia types, subtypes and normal bone marrow, respectively. Gene expression profiles were generated by means of PERL-programs, evaluated and plotted as bar graphs. Each of the analyzed groups are accordingly outlined. The following genes were selected and are given as FIGS. 432 to 464:

| GeneID | gene symbol | feature |
|---|---|---|
| 202503_s_at | KIAA0101 | CLL low |
| 202580_x_at | FOXM1 | CLL low |
| 202709_at | FMOD | CLL high |
| 204882_at | KIAA0053 | CLL high |
| 205049_s_at | CD79A | ALL high, CLL |
| | | high |
| 205051_s_at | KIT | AML high |
| 205382_s_at | DF | AML high |
| 205599_at | TRAF1 | CML low CLL high |
| 206255_at | BLK | ALL high, CLL |
| | | high |
| 206398_s_at | CD19 | ALL high, CLL |
| | | high |
| 210487_at | DNTT | ALL high |
| 210948_s_at | LEF1 | ALL high, CLL |
| | | high |
| 211352_s_at | NCOA3 | CLL high |
| 211404_s_at | APLP2 | AML high |
| 214761_at | OAZ | ALL high |
| 217950_at | NOSIP | CLL high |
| 218090_s_at | | CLL high |
| 218516_s_at | FLJ20421 | normal BM low |
| 218916_at | FLJ23436 | normal BM low |
| 219753_at | STAG3 | ALL high |
| 221969_at | PAX5 | ALL high, CLL |
| | | high |
| 223703_at | CDA017 | AML high, CML |
| | | high, normal BM |
| | | high |
| 226147_s_at | Homo sapiens cDNA: FLJ22667 | CLL high |
| | fis, clone HSI08385 | |
| 228471_at | ESTs | CLL high |
| 229487_at | ESTs | ALL high |
| 229790_at | TERF2 | CML low, BM low |
| 231736_x_at | MGST1 | AML high, CML |
| | | high, normal BM |
| | | high |
| 231854_at | Homo sapiens cDNA FLJ11448 | CML low |
| | fis, clone HEMBA1001391 | |
| 239287_at | ESTs | CLL high |
| 243362_s_at | LEF1 | ALL high |
| 243363_at | LEF1 | ALL high, CLL |
| | | high |
| 41577_at | PPP1R16B | CML low |

Tables 43a, b: functional gene annotation for genes identified to be differentially expressed between different types of leukemia, or between healthy bone marrow and leukemia, respectively.

As described by the GeneChip manufacturer, for each probeset (for example 200093_s_at_HG-U133A), a GenBank or RefSeq accession number was chosen to represent the target sequence. Using this accession number, a UniGene cluster (in current release) was identified where the accession number was used. If there is a link to LocusLink in the UniGene record, then annotations were retrieved from LocusLink. Those annotations include gene symbol, location, OMIM, EC, Gene Ontology (GO), description and RefSeq sequence accession. The RefSeq accession was linked to the protein annotations, which include domain identification (Pfam and BLOCKS), similarity search (blastp nr) and family classification (SCOP, EC and GPCR HMM searches).

Target sequence information for all the probes which were identified to be able to distinguish between different types and subtypes of leukemia and normal bone marrow, respectively, are given in Table 44.

As further described by the GeneChip manufacturer, the HG-U133 Target Databank is a compilation of probe set annotations and target sequence information for all the probes represented on the HG-U133 A and B arrays. Target sequences are the relatively short (typically around 300-600 bp) sequences against which probes have been designed on a GeneChip® array. These target sequences can be thought of as a subsequence of the Consensus/Exemplar sequence.

The Consensus/Exemplar sequences (i.e., the coding or full cDNA sequences corresponding to the markers described herein as being able to distinguish between different types and subtypes of leukemia and normal bone marrow) for most markers are given in Table 45.

### Example 7

### Conclusions

The expression pattern of genes allowed precise class assignments of defined leukemia types and subtypes according to the WHO classification of hematological malignancies, and normal BM, respectively.

Thus, we introduce candidate genes suitable for diagnosis of leukemia types and subtypes based on gene expression profiling.

These data demonstrate the utility of gene expression profiling for the discrimination of all leukemia major entities and most subentities. In total, up to 14 different leukemia types and subtypes could clearly be distinguished from each other and from normal BM, respectively. These leukemias are associated with highly differing prognoses and require specific treatment strategies. By performing these analyses on a single platform requiring basic molecular biological methods, this approach provides a broad access to high-quality diagnosis of leukemia.

TABLE A
Analysis of 18 samples class A versus 85 samples class non-A. On
the left the analysis according to Golub is presented for 20
informative genes. The crossvalidation accuracy is 0.87, confidence
0.77. Samples, where crossvalidation failed, are listed. For each
gene signal to noise ratio, p-value (significance obtained from
permutation test) and decision limit are provided. On the right the
same data set is analyzed using the protocol of the invention. By
selection of 3 genes (marked with asterisks) out of the top 20
genes and selecting optimized decision limits, the crossvalidation
accuracy reaches 0.96, confidence 0.88.

| Golub | | | | | | | |
|---|---|---|---|---|---|---|---|
| A - samples: 18/85 | | | | invention | | | |
| accuracy 0.87 | | | | A - samples: 18/85 | | | |
| confidence 0.77 | | | | accuracy 0.96 | | | |
| failed 6, 19, 22, 26, 78, | | | | confidence 0.88 | | | |
| 79, 80, 81, 82, 83, 84, 85, 99 | | | | failed 5, 6, 19, 22 | | | |
| | signal- | | decision | | | | decision |
| gene | to-noise | p | limit | gene | signal-to-noise | p | limit |
| g1 | −1.14 | 0* | 482.01 | g1 | −1.14 | 0 | |
| g2 | −1.06 | 0* | 192.17 | g2 | −1.06 | 0* | 98.50 |
| g3 | −0.97 | 0* | 207.67 | g3 | −0.97 | 0 | |
| g4 | 0.94 | 0* | 205.05 | g4 | 0.94 | 0 | |
| g5 | −0.93 | 0* | 1818.11 | g5 | −0.93 | 0 | |
| g6 | 0.93 | 0* | 451.74 | g6 | 0.93 | 0 | |
| g7 | −0.91 | 0* | 23.84 | g7 | −0.91 | 0 | |
| g8 | −0.90 | 0* | 225.72 | g8 | −0.90 | 0 | |
| g9 | 0.90 | 0* | 43.85 | g9 | 0.90 | 0 | |
| g10 | 0.89 | 0* | 210.78 | g10 | 0.89 | 0 | |
| g11 | −0.88 | 0* | 118.63 | g11 | −0.88 | 0 | |
| g12 | 0.87 | 0* | 55.39 | g12 | 0.87 | 0* | 67.80 |
| g13 | 0.87 | 0* | 127.15 | g13 | 0.87 | 0* | 164.10 |
| g14 | 0.86 | 0* | 222.04 | g14 | 0.86 | 0 | |
| g15 | 0.85 | 0* | 68.52 | g15 | 0.85 | 0 | |
| g16 | −0.85 | 0* | 546.97 | g16 | −0.85 | 0 | |
| g17 | 0.84 | 0* | 1242.77 | g17 | 0.84 | 0 | |
| g18 | −0.84 | 0* | 162.61 | g18 | −0.84 | 0 | |
| g19 | −0.83 | 0* | 385.39 | g19 | −0.83 | 0 | |
| g20 | 0.46 | 0* | 105.38 | g20 | 0.46 | 0 | |

LENGTHY TABLE REFERENCED HERE
US20070072178A1-20070329-T00001
Please refer to the end of the specification for access instructions.

LENGTHY TABLE REFERENCED HERE
US20070072178A1-20070329-T00002
Please refer to the end of the specification for access instructions.

LENGTHY TABLE REFERENCED HERE
US20070072178A1-20070329-T00003
Please refer to the end of the specification for access instructions.

LENGTHY TABLE REFERENCED HERE
US20070072178A1-20070329-T00004
Please refer to the end of the specification for access instructions.

LENGTHY TABLE REFERENCED HERE
US20070072178A1-20070329-T00005
Please refer to the end of the specification for access instructions.

LENGTHY TABLE REFERENCED HERE
US20070072178A1-20070329-T00006
Please refer to the end of the specification for access instructions.

LENGTHY TABLE REFERENCED HERE
US20070072178A1-20070329-T00007
Please refer to the end of the specification for access instructions.

LENGTHY TABLE REFERENCED HERE
US20070072178A1-20070329-T00008
Please refer to the end of the specification for access instructions.

LENGTHY TABLE REFERENCED HERE
US20070072178A1-20070329-T00009
Please refer to the end of the specification for access instructions.

LENGTHY TABLE REFERENCED HERE
US20070072178A1-20070329-T00010
Please refer to the end of the specification for access instructions.

LENGTHY TABLE REFERENCED HERE
US20070072178A1-20070329-T00011
Please refer to the end of the specification for access instructions.

LENGTHY TABLE REFERENCED HERE
US20070072178A1-20070329-T00012
Please refer to the end of the specification for access instructions.

LENGTHY TABLE REFERENCED HERE
US20070072178A1-20070329-T00013
Please refer to the end of the specification for access instructions.

LENGTHY TABLE REFERENCED HERE
US20070072178A1-20070329-T00014
Please refer to the end of the specification for access instructions.

LENGTHY TABLE REFERENCED HERE
US20070072178A1-20070329-T00015
Please refer to the end of the specification for access instructions.

LENGTHY TABLE REFERENCED HERE
US20070072178A1-20070329-T00016
Please refer to the end of the specification for access instructions.

LENGTHY TABLE REFERENCED HERE
US20070072178A1-20070329-T00017
Please refer to the end of the specification for access instructions.

LENGTHY TABLE REFERENCED HERE
US20070072178A1-20070329-T00018
Please refer to the end of the specification for access instructions.

LENGTHY TABLE REFERENCED HERE
US20070072178A1-20070329-T00019
Please refer to the end of the specification for access instructions.

LENGTHY TABLE REFERENCED HERE
US20070072178A1-20070329-T00020
Please refer to the end of the specification for access instructions.

LENGTHY TABLE REFERENCED HERE
US20070072178A1-20070329-T00021
Please refer to the end of the specification for access instructions.

LENGTHY TABLE REFERENCED HERE
US20070072178A1-20070329-T00022
Please refer to the end of the specification for access instructions.

LENGTHY TABLE REFERENCED HERE
US20070072178A1-20070329-T00023
Please refer to the end of the specification for access instructions.

LENGTHY TABLE REFERENCED HERE
US20070072178A1-20070329-T00024
Please refer to the end of the specification for access instructions.

LENGTHY TABLE REFERENCED HERE
US20070072178A1-20070329-T00025
Please refer to the end of the specification for access instructions.

LENGTHY TABLE REFERENCED HERE
US20070072178A1-20070329-T00026
Please refer to the end of the specification for access instructions.

LENGTHY TABLE REFERENCED HERE
US20070072178A1-20070329-T00027
Please refer to the end of the specification for access instructions.

LENGTHY TABLE REFERENCED HERE
US20070072178A1-20070329-T00028
Please refer to the end of the specification for access instructions.

LENGTHY TABLE REFERENCED HERE
US20070072178A1-20070329-T00029
Please refer to the end of the specification for access instructions.

LENGTHY TABLE REFERENCED HERE
US20070072178A1-20070329-T00030
Please refer to the end of the specification for access instructions.

LENGTHY TABLE REFERENCED HERE
US20070072178A1-20070329-T00031
Please refer to the end of the specification for access instructions.

LENGTHY TABLE REFERENCED HERE
US20070072178A1-20070329-T00032
Please refer to the end of the specification for access instructions.

LENGTHY TABLE REFERENCED HERE
US20070072178A1-20070329-T00033
Please refer to the end of the specification for access instructions.

LENGTHY TABLE REFERENCED HERE
US20070072178A1-20070329-T00034
Please refer to the end of the specification for access instructions.

LENGTHY TABLE REFERENCED HERE
US20070072178A1-20070329-T00035
Please refer to the end of the specification for access instructions.

LENGTHY TABLE REFERENCED HERE
US20070072178A1-20070329-T00036
Please refer to the end of the specification for access instructions.

LENGTHY TABLE REFERENCED HERE
US20070072178A1-20070329-T00037
Please refer to the end of the specification for access instructions.

LENGTHY TABLE REFERENCED HERE
US20070072178A1-20070329-T00038
Please refer to the end of the specification for access instructions.

LENGTHY TABLE REFERENCED HERE
US20070072178A1-20070329-T00039
Please refer to the end of the specification for access instructions.

LENGTHY TABLE REFERENCED HERE
US20070072178A1-20070329-T00040
Please refer to the end of the specification for access instructions.

LENGTHY TABLE REFERENCED HERE
US20070072178A1-20070329-T00041
Please refer to the end of the specification for access instructions.

LENGTHY TABLE REFERENCED HERE
US20070072178A1-20070329-T00042
Please refer to the end of the specification for access instructions.

LENGTHY TABLE REFERENCED HERE
US20070072178A1-20070329-T00043
Please refer to the end of the specification for access instructions.

LENGTHY TABLE REFERENCED HERE
US20070072178A1-20070329-T00044
Please refer to the end of the specification for access instructions.

LENGTHY TABLE REFERENCED HERE
US20070072178A1-20070329-T00045
Please refer to the end of the specification for access instructions.

LENGTHY TABLE REFERENCED HERE
US20070072178A1-20070329-T00046
Please refer to the end of the specification for access instructions.

LENGTHY TABLE REFERENCED HERE
US20070072178A1-20070329-T00047
Please refer to the end of the specification for access instructions.

LENGTHY TABLE REFERENCED HERE
US20070072178A1-20070329-T00048
Please refer to the end of the specification for access instructions.

LENGTHY TABLE REFERENCED HERE
US20070072178A1-20070329-T00049
Please refer to the end of the specification for access instructions.

LENGTHY TABLE REFERENCED HERE
US20070072178A1-20070329-T00050
Please refer to the end of the specification for access instructions.

LENGTHY TABLE REFERENCED HERE
US20070072178A1-20070329-T00051
Please refer to the end of the specification for access instructions.

LENGTHY TABLE REFERENCED HERE
US20070072178A1-20070329-T00052
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070072178A1). An electronic copy of the table will also be available from the USPTO upon request and payment of the fee set forth in 37 CFR 1.19(b)(3).

1. A method of determining whether a patient sample contains leukemia cells or other cells comprising the steps of
a) determining the expression profile of a group of markers in a patient sample and
b) concluding from expression profile whether the patient sample contains leukemia cells or other cells
characterized in
that the group of markers consists of markers selected independently from the markers listed in one or more of the tables 3 to 6, tables 15 to 20, tables 29, 30, 41, or 42 and whereby the number of markers in the group is between one and the total number of markers listed in the tables 3 to 6, tables 15 to 20, and tables 29, 30, 41, or 42.

2. The method according to claim 1
characterized in that
the number of markers in the group is between two and the total number of markers listed in the tables 3 to 6, tables 15 to 20, and tables 29, 30, 41, or 42.

3. The method according to claim 1 or 2
characterized in
that the group of markers consists of all markers listed in one or more tables, whereby the tables are selected from the tables 3 to 6, tables 15 to 20, and tables 29, 30, 41, or 42.

4. A method of determining whether a patient sample contains leukemia cells or other cells and at the same time or subsequently determining the type and subtype of leukemia cells, if leukemia cells are present, comprising the steps of
a) determining the expression profile of a group of markers in a patient sample and
b) concluding from the expression profile whether the patient sample contains leukemia cells or other cells and at the same time or subsequently determining the type and subtype of leukemia cells, if leukemia cells are present,
characterized in
that the group of markers consists of markers selected independently from the markers listed in one or more of the tables 16 to 20 or table 29 or 30 and whereby the number of markers in the group is between one and the total number of markers listed in the tables 16 to 20 or table 29 or 30.

5. The method according to claim 4
characterized in that
the number of markers in the group is between two and the total number of markers listed in the tables 16 to 20 or table 29 or 30.

6. The method according to claim 4 or 5
characterized in
that the group of markers consists of all markers listed in one or more tables,
whereby the tables are selected from the tables 16 to 20 or table 29 or 30.

7. The method according to any of claims 4 to 6
characterized in
that it is differentiated between four types of leukemia cells in the patient sample and that the other cells are normal cells.

8. The method according to any of claims 1 to 7
characterized in that at least one marker is selected from the group consisting of
ADCY3,
adenosine deaminase (ADA),
ARGHGAP4,
B-cell, a specific coactivator of octamer binding transcription factors,
CAPN3, a member of the papain superfamily,
CBFβ-MYH11,
CD24,
CD27,
CD74,
connective tissue growth factor (CTGF),
CTGF,
CTSW,
MYH11
glucocorticoid receptor beta
CBFA2T1 (formerly ETO)
HLA-DMB
HOXA9
HOXB5
IRF4, an immune system-restricted interferon regulatory factor
KIAA1013
LCN2, a modulator of inflammation
LEF-1
MBNL
MSF translocation partner of the mixed-lineage leukemia gene (MLL) in AML
NCOA1,
OS-9
Phospholipidscramblase 1 (PLSCR1) POU2AF1
POU2F2
POU4F1
SCYA3
DEFA3, SGP28, CAMP, CLC
SOCS-2 and
TRB and CD3D

9. A method of differentiating between two types of leukemia cells or one type of leukemia cells and normal cells or non-leukemia in a patient sample comprising the steps of
a) determining the expression profile of a group of markers in the patient sample and
b) concluding from the expression profile which type of leukemia cells the patient sample contains or whether it contains normal cells or non-leukemia
characterized in
that the group of markers consists of markers selected independently from the markers listed in one or more of the tables 3 to 6 or tables 7 to 12 and whereby the number of markers in the group is between one and the total number of markers listed in the tables 3 to 6 or tables 7 to 12.

10. The method according to claim 9
characterized in that
the number of markers in the group is between two and the total number of markers listed in one or more of the tables 3 to 6 or tables 7 to 12.

11. The method according to claim 9 or 10
characterized in
that the group of markers consists of all markers listed in one or more of the tables 3 to 6 or tables 7 to 12.

12. A method of differentiating between the subtypes of AML cells or the subtypes of AML cells and normal cells in a patient sample comprising the steps of
a) determining the expression profile of a group of markers in the patient sample and
b) concluding from the expression profile which subtypes of AML cells the patient sample contains or whether it contains normal cells
characterized in
that the group of markers consists of markers selected independently from the markers listed in one or more of the tables 1, 2, 13, 14, 17, 25, 27, 35 and 36 and whereby the number of markers in the group is between one and the total number of markers listed in one or more of the tables 1, 2, 13, 14, 17, 25, 27, 35 and 36.

13. The method according to claim 12
characterized in that
the number of markers in the group is between two and the total number of markers listed in the tables 1, 2, 13, 14, 17, 25, 27, 35 and 36.

14. The method according to claim 12 or 13
characterized in
that the group of markers consists of all markers listed in one or more of the tables 1, 2, 13, 14, 17, 25, 27, 35 and 36.

15. The method according to any of claims 12 to 14
characterized in
that three, four or more subtypes of AML cells are determined.

16. A method of assessing the efficacy of a test compound for inhibiting leukemia, the method comprising comparing the expression profile of a group of markers in a first sample obtained from the patient and maintained in the presence of the test compound and the expression profile of a group of markers in a second sample obtained from the patient and maintained in the absence of the test compound, wherein a significantly altered expression profile of the group of markers in the first sample, relative to the second sample, is an indication that the test compound is efficacious for inhibiting leukemia in the patient
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

17. A method of assessing the efficacy of a therapy for inhibiting leukemia in a patient, the method comprising comparing the expression profile of a group of markers in a first sample obtained from the patient prior to providing at least a portion of the therapy to the patient and the expression profile of a group of markers in a second sample obtained from the patient following provision of the portion of the therapy, wherein a significantly altered expression profile of the marker(s) in the second sample, relative to the first sample, is an indication that the therapy is efficacious for inhibiting leukemia in the patient
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

18. A method of selecting a composition for inhibiting leukemia in a patient, the method comprising:
a) separately maintaining aliquots of cells of a patient sample in the presence of a plurality of test compositions;
c) comparing the expression profile of a group of markers in each of the aliquots, and
d) selecting one of the test compositions which induces an altered expression profile of the group of markers in the aliquot containing that test composition, relative to other test compositions
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

19. A method of determining new subtypes of leukemia cells, the method comprising:
a) determining the expression profile of a group of markers of leukemia cells of unknown subtype
b) comparing the expression profile of said leukemia cells of ??? subtype to the expression profile of a group of markers of leukemia cells of known subtype(s), thereby concluding that a new subtype is determined when the expression profile is different to all known subtypes,
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

20. A method for guiding the therapy of leukemia in a patient depending on the leukemia subtype and/or the risk of relapse of disease, the method comprising:
a) determining the expression profile of a group of markers in the patient sample, and
b) deciding about the therapy strategy depending on the leukemia subtype and/or the risk of relapse of disease
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

21. A method for monitoring the progression of leukemia in a patient, the method comprising:
a) determining the expression profile of a group of markers in a patient sample at a first point in time, and
b) repeating step a) at a subsequent point in time; and
c) comparing the expression profile detected in steps a) and b), and therefrom monitoring the progression of leukemia in the patient,
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

22. The method of claim 21, wherein between the first point in time and the subsequent point in time, the patient has undergone chemotherapy.

23. The method according to any of the claims 1 to 22, wherein a transcribed polynucleotide or portion thereof is the marker or at least one of the markers.

24. The method of claim 23, wherein the transcribed polynucleotide is a mRNA.

25. The method of claim 23, wherein the transcribed polynucleotide is a cDNA.

26. The method according to any of claims 23 to 25, wherein the step of determining the expression profile further comprises amplifying the transcribed polynucleotide.

27. The method according to any of the claims 23 to 26, wherein the expression profile of the group of transcribed polynucleotides is determined by annealing the transcribed polynucleotides with a complementary polynucleotide or a portion thereof under stringent hybridization conditions.

28. The method according to any of the claims 1 to 27, wherein the patient sample is blood or bone marrow.

29. The method according to any of the claims 1 to 22, wherein a protein is the marker or at least one of the markers.

30. The method of claim 29, wherein the expression profile of the proteins is detected using a reagent which specifically binds to one of the proteins.

31. The method of claim 30, wherein the reagent is selected from the group consisting of an antibody, an antibody derivative, and an antibody fragment.

32. The method according to any of claims 16 to 31
characterized in that
the number of markers in the group is between two and the total number of markers listed in the tables 1 to 20, tables 25 or 27 or tables 29, 30, 32, 33, 35, 36, 38, 39, 41, 42.

33. The method according to any of claims 16 to 32
characterized in
that the group of markers consists of all markers listed in one or more of the tables 1 to 20, tables 25 or 27 or tables 29, 30, 32, 33, 35, 36, 38, 39, 41, 42.

34. A kit for assessing the suitability of each of a plurality of compounds for inhibiting leukemia in a patient, the kit comprising:
a) (optionally) a plurality of compounds; and
b) a reagent for assessing the expression profile of a group of markers
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

35. A kit for assessing whether a patient is afflicted with leukemia, the kit comprising reagents for assessing the expression profile of a group of markers
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

36. A kit for assessing the presence of human leukemia cells, the kit comprising an antibody, wherein the antibody specifically binds with a protein corresponding to a marker
characterized in
that the marker is selected from the tables 1 to 20, tables 25 or 27 or tables 29, 30, 32, 33, 35, 36, 38, 39, 41, 42.

37. A kit for assessing the leukemia cell carcinogenic potential of a test compound, the kit comprising leukemia cells and a reagent for assessing expression of a marker, wherein the marker is selected from the tables 1 to 20, tables 25 or 27 or tables 29, 30, 32, 33, 35, 36, 38, 39, 41, 42.

38. A protein or mRNA, cDNA or cRNA corresponding to a marker selected from the tables 1 to 20, tables 25 or 27 or tables 29, 30, 32, 33, 35, 36, 38, 39, 41, 42 for the treatment of leukemia.

39. A method for the preparation of a pharmaceutical composition for the treatment of leukemia
characterized in
that a protein corresponding to a marker selected from the tables 1 to 20, tables 25 or 27 or tables 29, 30, 32, 33, 35, 36, 38, 39, 41, 42 is admixed with
pharmaceutical compounds.

40. A method for the preparation of a pharmaceutical composition for the treatment of leukemia
characterized in
that a vector comprising a polynucleotide encoding a protein corresponding to a marker selected from the tables 1 to 20, tables 25 or 27 or tables 29, 30, 32, 33, 35, 36, 38, 39, 41, 42 is admixed with pharmaceutical compounds.

41. A method for the preparation of a pharmaceutical composition for the treatment of leukemia
characterized in
that an antisense oligonucleotide complementary to a polynucleotide encoding a protein corresponding to a marker selected from the tables 1 to 20, tables 25 or 27 or tables 29, 30, 32, 33, 35, 36, 38, 39, 41, 42 is admixed with pharmaceutical compounds.

42. Use of a marker or a group of markers selected individually from one or more of the tables 1 to 20, tables 25 or 27 or tables 29, 30, 32, 33, 35, 36, 38, 39, 41, 42 for the determination of leukemia cells, the type or subtype of leukemia cells.

43. Use of a marker or a group of markers selected individually from one or more of the tables 1, 2, 13, 14, 17, 25, 27, 35 or 36 for the determination of the subtype of AML cells.

44. A composition comprising a group of markers and substances chemically different to the markers
characterized in
that the group of markers consists of markers selected independently from the markers listed in one or more of the tables 1 to 20, tables 25 or 27 or tables 29, 30, 32, 33, 35, 36, 38, 39, 41, 42 and whereby the number of markers in the group is between one and the total number of markers listed in the tables 1 to 20, tables 25 or 27 or tables 29, 30, 32, 33, 35, 36, 38, 39, 41, 42.

45. The composition according to claim 44
characterized in
that the group of markers consists of all markers listed in one or more of the tables 1 to 20, tables 25 or 27 or tables 29, 30, 32, 33, 35, 36, 38, 39, 41, 42.

46. The composition according to claim 45
characterized in
that the group of markers consists of all markers listed in one or more of the tables 14 or tables 16 to 20 or table 29 or 30.

47. The composition according to claim 46
characterized in
that the group of markers consists of all markers listed in the tables 16 to 20 or table 29 or 30.

48. The composition according to claim 44 to 47
characterized in
that the markers are polynucleotides or oligonucleotides and are bound to a solid phase in the form of an array.

49. A method of determining the subtypes of ALL cells in a patient sample comprising the steps of
a) determining the level of expression of a group of markers in the patient sample and
b) concluding from the differences in the level of expression which subtypes of ALL cells the patient sample contains
characterized in
that the group of markers consists of markers selected independently from the markers listed in one or more of the tables 18, 32 or 33 and whereby the number of markers in the group is between two and the total number of markers listed in the tables 18, 32 or 33.

50. The method according to claim 49
characterized in
that the group of markers consists of all markers listed in one or more of the tables 18, 32 or 33.

51. A method of determining the subtypes of CLL cells in a patient sample comprising the steps of
a) determining the level of expression of a group of markers in the patient sample and
b) concluding from the differences in the level of expression which subtypes of CLL cells the patient sample contains
characterized in
that the group of markers consists of markers selected independently from the markers listed in one or more of the tables 38 or 39 and whereby the number of markers in the group is between two and the total number of markers listed in the tables 38 or 39.

52. The method according to claim 51
characterized in
that the group of markers consists of all markers listed in one or more of the tables

53. A method of determining whether a patient sample contains leukemia cells or other cells and at the same time determining the type and subtype of leukemia cells, comprising
a) providing a patient sample,
b) isolating RNA from the patient sample, transcribing the RNA into cDNA and transcribing the cDNA into cRNA while simultaneously labelling the cRNA
c) hybridising the cRNA to a microarray having attached thereto a group of markers selected from the group listed in one or more of the tables 1 to 20, tables 25 or 27 or tables 29, 30, 32, 33, 35, 36, 38, 39, 41, 42 and whereby the number of markers in the group is between two and the total number of markers listed in the tables 1 to 20, tables 25 or 27 or tables 29, 30, 32, 33, 35, 36, 38, 39, 41, 42, and
d) determining the level of expression of a marker or a group of markers.

54. Use of a marker or a group of markers selected from the group of members contained in the appended tables for determining whether a patient sample contains leukemia cells or other cells.

55. Use of claim 54 wherein said determination comprises the simultaneous determination of the type and subtype of leukemia cells.
